(19) United States
(12) Patent Application Publication
(10) Pub. No.: US 2008/0226645 A1
(43) Pub. Date: Sep. 18, 2008

(54) Methods and compositions for assessment and treatment of asthma

(75) Inventors: Margot Mary O'Toole, Newtonville, MA (US); Frederick William Immermann, Suffern, NY (US); Andrew Arthur Hill, Cambridge, MA (US); Padmalatha Sunkara Reddy, Lexington, MA (US); Michael Edward Burczynski, Collegeville, PA (US); Douglas Kenneth Miller, Collegeville, PA (US); Karl Henry Nocka, Harvard, MA (US); Stanley Francis Wolf, Arlington, MA (US); Michael Ronald Bowman, Westwood, MA (US); Andrew Joseph Dorner, Lexington, MA (US); I-Ming Wang, Seattle, WA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/008,691

(22) Filed: Jan. 10, 2008

Related U.S. Application Data
(60) Provisional application No. 60/879,994, filed on Jan. 10, 2007.

(51) Int. Cl. A61K 31/7088; C40B 40/06; A61K 39/395; C07K 16/00; A61K 31/7105; C07H 21/00; A61K 38/02; C40B 30/00

(52) U.S. Cl. 424/139.1; 506/16; 506/7; 514/44; 514/2; 530/387.9; 536/23.1

(57) ABSTRACT

The present invention provides methods and compositions for the assessment and treatment of asthma and other inflammatory diseases, particularly those mediated by interleukin-13 (IL-13). The present invention also provides arrays comprising markers for asthma as well as IL-13 responsiveness. The markers of the present invention can be used in methods to diagnose a patient as having asthma or an IL-13-mediated condition, to evaluate the effectiveness of potential therapeutic agents, to identify or evaluate agents capable of modulating marker expression levels, and to select a treatment for a patient suffering from asthma or an IL-13-mediated condition.

### TECHNICAL FIELD

The present invention relates to asthma markers and methods of using the same for the diagnosis, prognosis, and selection of biomarkers to assess effects of treatment and guide the treatment choice in asthma or other allergic or inflammatory diseases, particularly diseases mediated by interleukin-13 (IL-13) and fibrotic pathways modulated by the IL-13 pathway.

### BACKGROUND

Asthma is a complex, chronic inflammatory disease of the airways that is characterized by recurrent episodes of reversible airway obstruction, airway inflammation, and airway hyper responsiveness (AHR). Typical clinical manifestations include shortness of breath, wheezing, coughing, and chest tightness that can become life threatening or fatal. While existing therapies focus on reducing the symptomatic bronchospasm and pulmonary inflammation, there is growing awareness of the role of long-term airway remodeling in accelerated lung deterioration in asthmatics. Airway remodeling refers to a number of pathological features including epithelial smooth muscle and myofibroblast hyperplasia and/or metaplasia, subepithelial fibrosis and matrix deposition. The processes collectively result in up to about 300% thickening of the airway in cases of fatal asthma. Despite the considerable progress that has been made in elucidating the pathophysiology of asthma, the prevalence, morbidity and mortality of the disease has increased during the past two decades. In 1995, in the United States alone, nearly 1.8 million emergency room visits, 466,000 hospitalizations and 5,429 deaths were directly attributed to asthma. In fact, the prevalence of asthma has almost doubled in the past 20 years, with approximately 8-10% of the U.S. population affected by the disease (Cohn (2004) Annu. Rev. Immunol. 22:789-815). Worldwide, over four billion dollars is spent annually on treating asthma (Weiss (2001) J Allergy Clin. Immunol. 107:3-8).

It is generally accepted that allergic asthma is initiated by a dysregulated inflammatory reaction to airborne, environmental allergens. The lungs of asthmatics demonstrate an intense infiltration of lymphocytes, mast cells and eosinophils. This results in increased vascular permeability, smooth muscle contraction, bronchoconstriction, and inflammation. A large body of evidence has demonstrated this immune response is driven by CD4+ T-cells shifting their cytokine expression profile from T_{H}1 to a T_{H}2 cytokine profile (Maddox (2002) Annu. Rev. Med. 53:477-98). T_{H}2 cells mediate the inflammatory response through cytokine release, including interleukins (IL) leading to IgE production and release (Mosmann (1986) J Immunol 136:2348-57; Abbas (1996) Nature 383:787-93; Busse (2001) N. Engl. J. Med. 344:350-62). One murine model of asthma involves sensitization of the animal to ovalbumin (OVA) followed by intratracheal delivery of the OVA challenge. This procedure generates a T_{H}2 immune reaction in the mouse lung and mimics four major pathophysiological responses seen in human asthma, including upregulated serum IgE (atopy), eosinophilia, excessive mucus secretion, and AHR. The cytokine IL-13, expressed by basophils, mast cells, activated T cells and NK cells, plays a central role in the inflammatory response to OVA in mouse lungs. Direct lung instillation of murine IL-13 elicits all four of the asthma-related pathophysiologies and conversely, the presence of a soluble IL-13 antagonist (sIL-13Rα2-Fc) completely blocked both the OVA challenge-induced goblet cell mucus synthesis and the AHR to acetylcholine. Thus, IL-13-mediated signaling is sufficient to elicit all four asthma-related pathophysiological phenotypes and is required for the hypersecretion of mucus and induced AHR in the mouse model (Wills-Karp (2004) Immunol. Rev. 202:175-90).

Biologically active IL-13 binds specifically to a low-affinity binding chain IL-13Rα1 and to a high-affinity multimeric complex composed of IL-13Rα1 and IL-4R, a shared component of IL-4 signaling complex. The high-affinity complex is expressed in a wide variety of cell types including monocyte-macrophage populations, basophils, eosinophils, mast cells, endothelial cells, fibroblasts, airway smooth muscle cells, and airway epithelial cells. IL-13-mediated assembly of the functional receptor complex results in the phosphorylation-dependent activation of JAK1 and JAK2 or Tyk-2 kinases and IRS1/2 proteins. Activation of the IL-13 pathway cascade triggers the recruitment, phosphorylation and ultimate nuclear translocation of the transcriptional activator STAT6. A number of physiological studies demonstrate the inability of pulmonary OVA-challenge to elicit major pathology-related phenotypes including eosinophil infiltration, mucus hypersecretion, and airway hyperreactivity in mice homozygous for the STAT6^{-/-} null allele. Studies have indicated that polymorphisms in the IL-4/IL-13 cytokine-receptor signal transduction system may be indicative of disease predisposition and manifestations (Chatila (2004) Trends Mol. Med. 10(10):493-9). Recent genetic studies have also demonstrated a linkage between specific human alleles of IL-13 and its signaling components with asthma and atopy, demonstrating the critical role of this pathway in the human disease.

IL-13 also binds to an additional receptor chain, IL-13Rα2, which is expressed in both human and mouse. The murine IL-13Rα2 binds IL-13 with approximately 100-fold greater affinity (K_{d} of 0.5 to 1.2 nM) relative to IL-13Rα1, allowing the construction of a potent soluble IL-13 antagonist, sIL-13Rα2-Fc. The sIL-13Rα2-Fc has been used as an antagonist in a variety of disease models to demonstrate the role of IL-13 in Schistosomiasis induced liver fibrosis and granuloma formation, tumor immune surveillance, as well as in the OVA-challenge asthma model.

Current therapies for asthma are designed to inhibit the physiological processes associated with the dysregulated inflammatory responses associated with the diseases. Such therapies include the use of bronchodilators, corticosteroids, leukotriene inhibitors, and soluble IgE. Other treatments counter the airway remodeling occurring from bronchial airway narrowing, such as the bronchodilator salbutamol (Ventolin®), a short-acting B₂-agonist. (Barnes (2004) Nat. Rev. Drug Discov. 3:831-44; Boushey (1982) J. Allergy Clin. Immunol. 69: 335-8). The treatments share the same therapeutic goal of bronchodilation, reducing inflammation, and facilitating expectoration. Many of such treatments, however, include undesired side effects and lose effectiveness after being used for a period of time. Furthermore, current asthma treatments are not effective in all patients and relapse often occurs on these medications (van den Toorn (2001) Am. J. Respir. Crit. Care Med. 164:2107-13). Inter-individual variability in drug response and frequent adverse drug reactions to currently marketed drugs necessitate novel treatment strategies (Szefler (2002) J. Allergy Clin. Immunol. 109:410-8; Drazen (1996) N. Engl. J. Med. 335:841-7; Israel (2005) J. Allergy Clin. Immunol 115:S532-8; Lipworth (1999) Arch. Intern. Med. 159:941-55; Wooltorton (2005) CMAJ 173:1030-1; Guillot (2002) Expert Opin. Drug Saf. 1:325-9). Additionally, only limited agents for therapeutic intervention are available for decreasing the airway remodeling process that occurs in asthmatics. Therefore, there remains a need for an increased molecular understanding of the pathogenesis and etiology of asthma, and a need for the identification of novel therapeutic strategies to combat these complex diseases.

### SUMMARY OF THE INVENTION

The present invention provides markers which are related to genes expressed at abnormal levels in the blood of asthma subjects, and these include genes that are involved in the IL-13 pathway. Dysregulation of the IL-13 pathway, as noted above, has been strongly implicated in animal models of asthma. However, the present invention includes markers, a number of which are genes that can be measured in the blood, and are expressed in the blood at significantly different levels in asthma and healthy subjects. The present invention also includes markers that are responsive to variation in the level of IL-13, and have their expression levels modulated by the presence of IL-13 or an IL-13 antagonist. The present invention also includes markers, a number of which are transcriptional biomarkers that are related to asthma but are not known to be involved in the IL-13 pathway. The markers of the present invention have utility in assessing whether a therapy modulates their expression levels toward a healthy level. These biomarkers are also of potential utility in the diagnosis, prognosis, or assessment of inflammatory diseases other than asthma, including IL-13-mediated conditions.

The present invention provides markers for asthma. Those markers can be used, for example, in the evaluation of a patient or in the identification of agents capable of modulating their expression; such agents may also be useful clinically.

The present invention also provides markers for IL-13 responsiveness. Those markers can be used, for example, in the evaluation of a patient or in the identification of agents capable of modulating their expression; such agents may also be useful clinically.

Thus, in one aspect, the present invention provides a method for providing a diagnosis, prognosis, or assessment for an individual afflicted with asthma or an IL-13-mediated condition. The method includes the following steps: (1) detecting the expression levels of one or more differentially expressed genes, or markers, of asthma or IL-13 responsiveness in a sample derived from a patient prior to the treatment; and (2) comparing each of the expression levels to a corresponding control, or reference, expression level for the marker. Diagnosis or other assessment is based, in whole or in part, on the outcome of the comparison. In one embodiment, the determination as to whether a treatment significantly affects the expression levels of one or more markers uses standard controls and normalizers. In some embodiments, the determination is based on a comparison of the expression level, for example, to a numerical threshold, to a level indicative of an asthma state, to a level in the same patient at a different time point, or to a level in the same patient before or during a treatment regimen.

In some embodiments, the reference expression level is a level indicative of the presence of asthma. In other embodiments, the reference expression level is a level indicative of the absence of asthma. In some embodiments, the reference expression level is a level indicative of responsiveness to IL-13. In other embodiments, the reference expression level is a numerical threshold, which can be chosen, for example, to distinguish between the presence and absence of asthma. In still other embodiments, the reference expression level is a numerical threshold, which can be chosen to distinguish between the presence and absence of IL-13 responsiveness. In other embodiments, the reference expression level is an expression level from a sample from the same individual but the sample is taken at, for example, a different time, such as with regard to administration of a treatment or progression of a disease.

In another aspect of the present invention, what is provided is a method for diagnosing a patient as having asthma including comparing the expression level of a marker in the patient to a reference expression level of the marker and diagnosing the patient has having asthma if there is a significant difference in the expression levels observed in the comparison. In another aspect of the present invention, what is provided is a method for determining the responsiveness of markers to IL-13 exposure including comparing the expression level of a marker in the patient to a reference expression level of the marker.

In a further aspect of the invention, what is provided is a method for evaluating the effectiveness of a treatment for asthma or an IL-13-mediated condition including the steps of (1) detecting the expression levels of one or more differentially expressed genes, or markers, of asthma or an IL-13-mediated condition in a sample derived from a patient during the course of the treatment; and (2) comparing each of the expression levels to a corresponding control, or reference, expression level for the marker, wherein the result of the comparison is indicative of the effectiveness of the treatment.

In another aspect of the present invention, what is provided is a method for selecting a treatment for asthma in a patient involving the steps of (1) detecting an expression level of a marker in a sample derived from the patient; (2) comparing the expression level of the marker to a reference expression level of the marker; and (3) diagnosing the patient as having a type of asthma likely to be responsive to a particular therapeutic strategy; and (4) selecting a treatment for the patient.

In another aspect of the present invention, what is provided is a method for detecting exposure to IL-13 or an IL-13 antagonist involving the steps of (1) detecting an expression level of a marker in one or more cells; and (2) comparing the expression level of the marker to a reference expression level of the marker; wherein the comparison of the expression levels indicates exposure to IL-13 or an IL-13 antagonist. In one aspect, the method of detecting exposure to IL-13, an IL-13 antagonist, or an IL-13 agonist comprises the steps of detecting a level of expression of at least one marker in one or more cells; and comparing the level of expression of the at least one marker to a reference level of expression of the at least one marker; wherein a difference in the level of expression of the at least one marker and the reference level of expression is indicative of exposure to IL-13, an IL-13 antagonist, or an IL-13 agonist; and wherein the at least one marker is selected from the group consisting of the markers indicated in Table 7.

The present invention further provides a method for modulating an inflammatory disease comprising providing an agent that binds to at least one marker gene product of the present invention. In one embodiment, the marker is selected from Table 1a and b. In one embodiment, the marker is selected from the markers in Table 1b wherein “yes” is indicated in Column C. In a further embodiment of the present invention, the marker is one of the 5 unknown/not previously characterized genes. In one embodiment, the disease is asthma. In another embodiment of the present invention, the disease is an IL-13-mediated condition. The agent may be a nucleic acid comprising the markers in Table 2, a nucleic acid complementary to a nucleic acid marker from Table 2, an SiRNA, an isolated antibody to a polypeptide from Table 2, an isolated nucleic acid comprising a nucleic acid from Table 2, or an isolated polypeptide from Table 2

The present invention further provides a method for modulating an inflammatory disease comprising providing an agent that modulates the level of expression of at least one marker of the present invention. In one embodiment, the marker is selected from Table 1a and b. In a further embodiment of the present invention, the marker is one of the 5 unknown/not previously characterized genes. In one embodiment, the disease is asthma. In another embodiment of the present invention, the disease is an IL-13-mediated condition.

In a further aspect of the present invention, what is provided is a method for evaluating agents capable of modulating the expression of a marker that is differentially expressed in asthma or is responsive to IL-13 involving the steps of (1) contacting one or more cells with the agent, or optionally, administering the agent to a human or non-human mammal; (2) determining the expression level of the marker; and (3) comparing the expression level of the marker to the expression level of the marker in an untreated cell or untreated human or untreated non-human mammal. The comparison is indicative of the agent's ability to modulate the expression level of the marker in question.

“Diagnostic genes” or “markers” or “prognostic genes” referred to in the application include, but are not limited to, any genes or gene fragments that are differentially expressed in peripheral blood mononuclear cells (PBMCs) or other tissues of subjects having asthma as compared to the expression of said genes in an otherwise healthy individual. Exemplary markers are shown in Table 1a and b. It is often the case that there is differential expression of a marker between patients with different clinical outcomes. Markers include genes whose expression levels in PBMCs or other tissues of asthma patients or patients having an IL-13-mediated condition are correlated with clinical outcomes of the patients. A “clinical outcome” referred to in the application includes, but is not limited to, any response to any asthma-related or IL-13-mediated condition-related treatment.

In some embodiments, each of the expression levels of the marker is compared to a corresponding control level which is a numerical threshold. The numerical threshold can be, for example, a ratio, a difference, a confidence level, or another quantitative indicator.

In another aspect, the present invention provides a method for predicting a clinical outcome of asthma or an IL-13-mediated condition including the following steps: (1) generating a gene expression profile from a peripheral blood sample of a patient having asthma or an IL-13-mediated condition; and (2) comparing the gene expression profile to one or more reference expression profiles. The gene expression profile and the one or more reference expression profiles contain expression patterns of one or more markers of the asthma or IL-13-mediated condition in PBMCs. The difference or similarity between the gene expression profile and the one or more reference expression profiles is indicative of the clinical outcome for the patient.

In one embodiment, the gene expression profile of the one or more markers may be compared to the one or more reference expression profiles by, for example, a k-nearest neighbor analysis or a weighted voting algorithm. Typically, the one or more reference expression profiles represent known or determinable clinical outcomes. In some embodiments, the gene expression profile from the patient may be compared to at least two reference expression profiles, each of which represents a different clinical outcome. In some embodiments, one or more reference expression profiles may include a reference expression profile representing a patient without asthma.

In some embodiments, the gene expression profile may be generated by using a nucleic acid array. Typically, the gene expression profile is generated from the peripheral blood sample of the patient prior to therapy for asthma. Alternatively, the gene expression profile is generated from the peripheral blood sample of a patient exposed to IL-13 or an IL-13 antagonist.

In one embodiment, the one or more markers include one or more genes selected from Table 1a and b. In another embodiment, the one or more markers include ten or more genes selected from Table 1a and b. In yet another embodiment, the one or more markers include twenty or more genes selected from Table 1a and b. In one embodiment, the one or more markers are selected from the markers in Table 1b wherein “yes” is indicated in Column C.

In yet another aspect, the present invention provides a method for selecting a treatment for an asthma patient. The method includes the following steps: (1) generating a gene expression profile from a peripheral blood sample derived from the asthma patient; (2) comparing the gene expression profile to a plurality of reference expression profiles, each representing a clinical outcome in response to one of a plurality of treatments; and (3) selecting from the plurality of treatments a treatment which has a favorable clinical outcome for the asthma patient. The treatment selection of step (3) is based on the comparison in step (2), wherein the gene expression profile and the one or more reference expression profiles comprise expression patterns of one or more markers of the asthma in PBMCs. In one embodiment, the gene expression profile may be compared to a plurality of reference expression profiles by, for example, a k-nearest neighbor analysis or a weighted voting algorithm.

In one embodiment, the one or more markers include one or more genes selected from Table 1a and b. In another embodiment, the one or more markers include ten or more genes selected from Table 1a and b. In yet another embodiment, the one or more markers include twenty or more genes selected from Table 1a and b. In one embodiment, the one or more markers are selected from the markers in Table 1b wherein “yes” is indicated in Column C.

In another aspect, the present invention provides a method for diagnosis, assessment, prognosis, or monitoring the occurrence, development, progression, or treatment of asthma. The present invention also provides a method for diagnosis, assessment, prognosis, or monitoring the occurrence, development, progression, or treatment of an IL-13-mediated condition. The method includes the following steps: (1) generating a gene expression profile from a peripheral blood sample of a patient having asthma or an IL-13-mediated condition; and (2) comparing the gene expression profile to one or more reference expression profiles, wherein the gene expression profile and the one or more reference expression profiles contain the expression patterns of one or more markers of asthma or an IL-13-mediated condition in PBMCs, or other tissues, and wherein the difference or similarity between the gene expression profile and the one or more reference expression profiles is indicative of the presence, absence, occurrence, development, progression, or effectiveness of treatment of the asthma or an IL-13-mediated condition in the patient. In one embodiment, the disease is asthma. In one aspect, the invention provides a method for selecting a treatment for an asthma patient comprising generating a sample expression profile from a sample derived from the asthma patient; comparing the sample expression profile to at least one reference expression profile, wherein the at least one reference expression profile represents a favorable clinical outcome in response to a treatment; selecting a treatment; wherein the treatment is one that exhibits a reference expression profile that is different from the sample expression profile; and wherein the sample expression profile and the at least one reference expression profile comprise an expression profile of a marker indicated in Table 1a or Table 1b.

Typically, the one or more reference expression profiles include a reference expression profile representing a disease-free human. Typically, the one or more markers include one or more genes selected from Table 1a and b. In some embodiments, the one or more markers include ten or more genes selected from Table 1a and b. In one embodiment, the one or more markers are selected from the markers in Table 1b wherein “yes” is indicated in Column C.

In another aspect, the present invention provides an array for detecting a marker differentially expressed in asthma or responsive to exposure to IL-13. In another embodiment, the array is for use in a method for predicting a clinical outcome for an asthma patient. The array of the invention includes a substrate having a plurality of addresses, each of which has a distinct probe disposed thereon or affixed thereto. In one embodiment, at least 10% of the plurality of addresses have affixed thereto or disposed thereon probes that can specifically detect or hybridize to markers for asthma or IL-13 responsiveness. In some embodiments, at least 15% of the plurality of addresses have disposed thereon or affixed thereto probes that can specifically detect or hybridize to markers of asthma or IL-13 responsiveness in PBMCs or other tissues. In some embodiments, at least 20% of the plurality of addresses have disposed thereon or affixed thereto probes that can specifically detect or hybridize to markers of asthma or IL-13 responsiveness in PBMCs or other tissues. In some embodiments, at least 25% of the plurality of addresses have disposed thereon or affixed thereto probes that can specifically detect or hybridize to markers of asthma or IL-13 responsiveness in PBMCs or other tissues. In some embodiments, at least 30% of the plurality of addresses have disposed thereon or affixed thereto probes that can specifically detect or hybridize to markers of IL-13 responsiveness or asthma in PBMCs or other tissues. In some embodiments, at least 40% of the plurality of addresses have disposed thereon or affixed thereto probes that can specifically detect or hybridize to markers of asthma or IL-13 responsiveness in PBMCs or other tissues. In some embodiments, at least 50% of the plurality of addresses have disposed thereon or affixed thereto probes that can specifically detect or hybridize to markers of IL-13 responsiveness or asthma in PBMCs or other tissues. In some embodiments, at least 60% of the plurality of addresses have disposed thereon or affixed thereto probes that can specifically detect or hybridize to markers of asthma or IL-13 responsiveness in PBMCs or other tissues. In some embodiments, at least 70% of the plurality of addresses have disposed thereon or affixed thereto probes that can specifically detect or hybridize to markers of asthma or IL-13 responsiveness in PBMCs or other tissues. In some embodiments, at least 80% of the plurality of addresses have disposed thereon or affixed thereto probes that can specifically detect or hybridize to markers of asthma or IL-13 responsiveness in PBMCs or other tissues. In some embodiments, at least 90% of the plurality of addresses have disposed thereon or affixed thereto probes that can specifically detect or hybridize to markers of asthma or IL-13 responsiveness in PBMCs or other tissues. In some embodiments, the markers are selected from Table 1a and b. In other embodiments, the markers are selected from the markers in Table 1b wherein “yes” is indicated in Column C. In some embodiments, at least 10%, 15%, 20%, 30%, 40%, 50%, 60%, 70%, 80%, or at least 90% of the plurality of addresses have disposed thereon or affixed thereto markers selected from Table 1a and b. In some embodiments, at least 10%, 15%, 20%, 30%, 40%, 50%, 60%, 70%, 80%, or at least 90% of the plurality of addresses have disposed thereon or affixed thereto markers selected from the markers in Table 1b wherein “yes” is indicated in Column C. In some embodiments, the array of the present invention has affixed to or disposed thereon at least 5, preferably at least 10, at least 15, at least 20, at least 25, at least 30, at least 35, at least 40, at least 45, at least 50, at least 60, at least 70, at least 80, at least 90, at least 100, or at least 150 markers selected from Table 1a and b. In some embodiments, the array of the present invention has affixed to or disposed thereon at least 5, preferably at least 10, at least 15, at least 20, at least 25, at least 30, at least 35, at least 40, at least 45, at least 50, at least 60, or at least 70 markers selected from Table 1b wherein “yes” is indicated in Column C. The probe suitable for the present invention may be a nucleic acid probe. Alternatively, the probe suitable for the present invention may be an antibody probe.

In a further aspect, the present invention provides an array for use in a method for diagnosis of asthma or an IL-13-mediated condition including a substrate having a plurality of addresses, each of which have a distinct probe disposed thereon or affixed thereto. In one embodiment, at least 10% of the plurality of addresses have affixed thereto or disposed thereon probes that can specifically detect or hybridize to markers for asthma or IL-13 responsiveness. In some embodiments, at least 15% of the plurality of addresses have disposed thereon or affixed thereto probes that can specifically detect or hybridize to markers of asthma or IL-13 responsiveness in PBMCs or other tissues. In some embodiments, at least 20% of the plurality of addresses have disposed thereon or affixed thereto probes that can specifically detect or hybridize to markers of asthma or IL-13 responsiveness in PBMCs or other tissues. In some embodiments, at least 25% of the plurality of addresses have disposed thereon or affixed thereto probes that can specifically detect or hybridize to markers of asthma or IL-13 responsiveness in PBMCs or other tissues. In some embodiments, at least 30% of the plurality of addresses have disposed thereon or affixed thereto probes that can specifically detect or hybridize to markers of IL-13 responsiveness or asthma in PBMCs or other tissues. In some embodiments, at least 40% of the plurality of addresses have disposed thereon or affixed thereto probes that can specifically detect or hybridize to markers of asthma or IL-13 responsiveness in PBMCs or other tissues. In some embodiments, at least 50% of the plurality of addresses have disposed thereon or affixed thereto probes that can specifically detect or hybridize to markers of IL-13 responsiveness or asthma in PBMCs or other tissues. In some embodiments, at least 60% of the plurality of addresses have disposed thereon or affixed thereto probes that can specifically detect or hybridize to markers of asthma or IL-13 responsiveness in PBMCs or other tissues. In some embodiments, at least 70% of the plurality of addresses have disposed thereon or affixed thereto probes that can specifically detect or hybridize to markers of asthma or IL-13 responsiveness in PBMCs or other tissues. In some embodiments, at least 80% of the plurality of addresses have disposed thereon or affixed thereto probes that can specifically detect or hybridize to markers of asthma or IL-13 responsiveness in PBMCs or other tissues. In some embodiments, at least 90% of the plurality of addresses have disposed thereon or affixed thereto probes that can specifically detect or hybridize to markers of asthma or IL-13 responsiveness in PBMCs or other tissues. In some embodiments, the markers are selected from Table 1a and b. In other embodiments, the markers are selected from the markers in Table 1b wherein “yes” is indicated in Column C. In some embodiments, at least 10%, 15%, 20%, 30%, 40%, 50%, 60%, 70%, 80%, or at least 90% of the plurality of addresses have disposed thereon or affixed thereto markers selected from Table 1a and b. In some embodiments, at least 10%, 15%, 20%, 30%, 40%, 50%, 60%, 70%, 80%, or at least 90% of the plurality of addresses have disposed thereon or affixed thereto markers selected from the markers in Table 1b wherein “yes” is indicated in Column C. In some embodiments, the array of the present invention has affixed to or disposed thereon at least 5, preferably at least 10, at least 15, at least 20, at least 25, at least 30, at least 35, at least 40, at least 45, at least 50, at least 60, at least 70, at least 80, at least 90, at least 100, or at least 150 markers selected from Table 1a and b. In some embodiments, the array of the present invention has affixed to or disposed thereon at least 5, preferably at least 10, at least 15, at least 20, at least 25, at least 30, at least 35, at least 40, at least 45, at least 50, at least 60, or at least 70 markers selected from Table 1b wherein “yes” is indicated in Column C. The probe suitable for the present invention may be a nucleic acid probe. Alternatively, the probe suitable for the present invention may be an antibody probe.

In a further aspect, the present invention provides a low density array for use in a method of diagnosis, prognosis, or assessment of asthma or an IL-13-mediated condition or determination of IL-13 responsiveness, including a substrate having a plurality of addresses, each of which has a distinct probe disposed thereon or affixed thereto. The low density array provides the benefit of lower cost, given the lower number of probes that are required to be disposed upon or affixed to the array. Furthermore, the low density array also provides a higher sensitivity given the greater representation of a select number of probes of interest as a percentage of all probes at all addresses on the array. In one embodiment, the present invention provides a low density array for use in assessing a patient's asthma or IL-13-mediated condition or IL-13 responsiveness. In another embodiment, the present invention provides a low density array for use in evaluating or identifying agents capable of modulating the level of expression of markers that are differentially expressed in asthma or IL-13-mediated condition or are responsive to IL-13. In one embodiment, the low density array is capable of hybridizing to at least 10 markers selected from Table 1a and b. In another embodiment, the low density array is capable of hybridizing to at least 20 markers selected from Table 1a and b. In one embodiment, at least 10% of the plurality of addresses have affixed thereto or disposed thereon probes that can specifically detect or hybridize to markers for asthma or IL-13 responsiveness. In some embodiments, at least 15% of the plurality of addresses have disposed thereon or affixed thereto probes that can specifically detect or hybridize to markers of asthma or IL-13 responsiveness in PBMCs or other tissues. In some embodiments, at least 20% of the plurality of addresses have disposed thereon or affixed thereto probes that can specifically detect or hybridize to markers of asthma or IL-13 responsiveness in PBMCs or other tissues. In some embodiments, at least 25% of the plurality of addresses have disposed thereon or affixed thereto probes that can specifically detect or hybridize to markers of asthma or IL-13 responsiveness in PBMCs or other tissues. In some embodiments, at least 30% of the plurality of addresses have disposed thereon or affixed thereto probes that can specifically detect or hybridize to markers of IL-13 responsiveness or asthma in PBMCs or other tissues. In some embodiments, at least 40% of the plurality of addresses have disposed thereon or affixed thereto probes that can specifically detect or hybridize to markers of asthma or IL-13 responsiveness in PBMCs or other tissues. In some embodiments, at least 50% of the plurality of addresses have disposed thereon or affixed thereto probes that can specifically detect or hybridize to markers of IL-13 responsiveness or asthma in PBMCs or other tissues. In some embodiments, at least 60% of the plurality of addresses have disposed thereon or affixed thereto probes that can specifically detect or hybridize to markers of asthma or IL-13 responsiveness in PBMCs or other tissues. In some embodiments, at least 70% of the plurality of addresses have disposed thereon or affixed thereto probes that can specifically detect or hybridize to markers of asthma or IL-13 responsiveness in PBMCs or other tissues. In some embodiments, at least 80% of the plurality of addresses have disposed thereon or affixed thereto probes that can specifically detect or hybridize to markers of asthma or IL-13 responsiveness in PBMCs or other tissues. In some embodiments, at least 90% of the plurality of addresses have disposed thereon or affixed thereto probes that can specifically detect or hybridize to markers of asthma or IL-13 responsiveness in PBMCs or other tissues. In some embodiments, the markers are selected from Table 1a and b. In other embodiments, the markers are selected from the markers in Table 1b wherein “yes” is indicated in Column C. In some embodiments, at least 10%, 15%, 20%, 30%, 40%, 50%, 60%, 70%, 80%, or at least 90% of the plurality of addresses have disposed thereon or affixed thereto markers selected from Table 1a and b. In some embodiments, at least 10%, 15%, 20%, 30%, 40%, 50%, 60%, 70%, 80%, or at least 90% of the plurality of addresses have disposed thereon or affixed thereto markers selected from the markers in Table 1b wherein “yes” is indicated in Column C. In some embodiments, the array of the present invention has affixed to or disposed thereon at least 5, preferably at least 10, at least 15, at least 20, at least 25, at least 30, at least 35, at least 40, at least 45, at least 50, at least 60, at least 70, at least 80, at least 90, at least 100, or at least 150 markers selected from Table 1a and b. In some embodiments, the array of the present invention has affixed to or disposed thereon at least 5, preferably at least 10, at least 15, at least 20, at least 25, at least 30, at least 35, at least 40, at least 45, at least 50, at least 60, or at least 70 markers selected from Table 1b wherein “yes” is indicated in Column C. The probe suitable for the present invention may be a nucleic acid probe. Alternatively, the probe suitable for the present invention may be an antibody probe.

In yet another aspect, the present invention provides a computer-readable medium containing a digitally-encoded expression profile having a plurality of digitally-encoded expression signals, each of which includes a value representing the expression of a marker for asthma or IL-13 responsiveness in a PBMC, or in another tissue. In some embodiments, each of the plurality of digitally-encoded expression signals has a value representing the expression of the marker for asthma or IL-13 responsiveness in a PBMC, or another tissue, of a patient with a known or determinable clinical outcome. In some embodiments, the computer-readable medium of the present invention contains a digitally-encoded expression profile including at least ten digitally-encoded expression signals.

In another aspect, the present invention provides a computer-readable medium containing a digitally-encoded expression profile having a plurality of digitally-encoded expression signals, each of which has a value representing the expression of a marker for asthma or IL-13 responsiveness in a PBMC or other tissue. In some embodiments, each of the plurality of digitally-encoded expression signals has a value representing the expression of the marker of asthma or IL-13 responsiveness in a PBMC, or another tissue, of an asthma-free human or non-human mammal. In some embodiments, the computer-readable medium of the present invention contains a digitally-encoded expression profile including at least ten digitally-encoded expression signals.

In yet another aspect, the present invention provides a kit for prognosis of asthma or an IL-13-mediated condition. The kit includes a) one or more probes that can specifically detect markers for asthma or IL-13 responsiveness in PBMCs, or another tissue; and b) one or more controls, each representing a reference expression level of a marker detectable by the one or more probes. In some embodiments, the kit of the present invention includes one or more probes that can specifically detect markers selected from Table 1a and b. In some embodiments, the kit of the present invention includes one or more probes that can specifically detect markers selected from the markers in Table 1b wherein “yes” is indicated in Column C.

In yet another aspect, the present invention provides a kit for diagnosis of asthma or an IL-13-mediated condition. The kit includes a) one or more probes that can specifically detect markers of asthma or IL-13 responsiveness in PBMCs, or another tissue; and b) one or more controls, each representing a reference expression level of a marker detectable by the one or more probes. In some embodiments, the kit of the present invention includes one or more probes that can specifically detect markers selected from Table 1a and b. In some embodiments, the kit of the present invention includes one or more probes that can specifically detect markers selected from the markers in Table 1b wherein “yes” is indicated in Column C.

In one embodiment, the sample contains protein molecules from the test subject. Alternatively, the biological sample can contain mRNA molecules from the test subject or genomic DNA molecules from the test subject. An exemplary biological sample is a peripheral blood sample isolated by conventional means from a subject, e.g., blood draw. Alternatively, the sample can comprise tissue, mucus, or cells isolated by conventional means from a subject, e.g., biopsy, swab, surgery, endoscopy, bronchoscopy, and other techniques well known to the skilled artisan.

Other features, objects, and advantages of the present invention are apparent in the detailed description that follows. It should be understood, however, that the detailed description, while indicating embodiments of the present invention, is given by way of illustration only and not by way of limitation. Various changes and modifications within the scope of the invention will become apparent to those skilled in the art from the detailed description.

### DETAILED DESCRIPTION

The present invention provides methods useful for the diagnosis and assessment of asthma as well as the selection of a treatment for asthma after its assessment. The present invention further provides methods useful for the diagnosis and assessment of IL-13 responsiveness, including an IL-13-mediated condition. The terms “IL-13 responsiveness,” “IL-13 responsive,” and “responsive to IL-13” as used herein refer to a marker or gene's modulation in reaction to exposure to IL-13, an IL-13 antagonist, an IL-13 agonist, or the like. These methods employ asthma and IL-13 responsive markers which are differentially expressed in tissue samples, particularly, peripheral blood samples, of asthma patients or patients with an IL-13-mediated condition who have different clinical outcomes. The present invention also provides methods for monitoring the occurrence, development, progression, effectiveness of a treatment, or treatment of asthma or an IL-13-mediated condition. The present invention further provides methods for offering a prognosis or determining the efficacy of treatment for asthma or an IL-13-mediated condition using the disclosed asthma and IL-13 responsive markers which are differentially expressed in peripheral blood samples, or other tissues, of asthma patients, or patients with an IL-13-mediated condition, with different disease status. Thus, the present invention represents a significant advance in clinical asthma pharmacogenomics and asthma treatment as well as the clinical pharmacogenomics and treatment of conditions mediated by IL-13, including inflammatory disease.

Various aspects of the invention are described in further detail in the following subsections. The use of subsections is not meant to limit the invention. Each subsection may apply to any aspect of the invention. In this application, the use of “or” means “and/or” unless stated otherwise.

### Identification of Asthma Markers for the Taqman Low Density Array (TLDA)

Analyses were performed to select 167 genes as the top candidate markers to assess the effects of IMA638, an IL-13 antagonist, by Taqman Low Density Array (TLDA). Using a dataset consisting of HG-U133A GeneChip® (Affymetrix) results from 1147 individual visits from 337 non-smoking asthma subjects and 1183 visits from 348 non-smoking healthy subjects, ANCOVA analyses were performed to identify genes that, by gene expression level, were most significantly associated with asthma and, on an individual visit basis, showed the highest incidence of a detectable fold change when compared to the average level in healthy subjects.

The list of genes thus identified were compared to lists from three independent in vitro studies, two that identified gene expression changes resulting from exposure of human monocytes to IL-13, and a third that identified the effects of IL-13 antagonism on the 6 day PBMC response to allergen stimulation. Also taken into consideration were the results of two in vivo animal studies—one that identified genes affected by IL-13 instillation in the mouse lung, and the other that identified changes in gene expression levels in PBMCs associated with segmental ascaris lung challenge of non-human primates.

In assigning slots on the TLDA, highest priority was given to genes significantly (i.e., having a false discovery rate, or FDR, of less than 1.0e-5) and consistently (in more than 59% of samples) associated with asthma by gene expression level in PBMC and had an average GeneChip® signal greater than 30, and were significantly (FDR<0.05) affected in vitro by IL-13 or its antagonist. A total of 71 genes met all these requirements and are indicated as having met these requirements with a “yes” in Column C of Table 1b.

The vast majority of the remaining TLDA slots were assigned to genes showing a very highly significant (FDA<1.0e-5) association with asthma by expression levels in PBMC and met at least one of the following criteria: a) average fold change of >1.4 in the comparison of asthma and healthy subjects; b) average fold change >1.25, with intra-subject variability <35% and more than 59% of samples showing an expression level difference with the average of healthy volunteers; and/or c) intra-subject variability <20% and more than 59% of samples showing a detectable expression level difference with the average of healthy volunteers. The remaining slots were assigned to genes that were associated with IL-13 through either the in vitro or animal model studies, even if the incidence of samples that differed from the healthy subject average was less than 59% and the association with asthma did not meet the FDR<1.0e-5 level of significance. Table 1a and b provides a complete list of the genes selected as having satisfied the aforementioned criteria and includes the identities and descriptions of the genes as well as pertinent statistical information. The sequences of the probes identified in Table 1a and b are provided in Table 6.

Table 1a provides the Affymetrix Gene Symbol, gene description and Affymetrix Qualifiers for each marker in columns A, B, and C, respectively. Column D discloses the raw p value for association with asthma when gene expression levels in 1147 samples from 337 asthma subjects were compared to levels in 1183 samples from 348 healthy subjects. ANCOVA was performed to adjust for covariates related to age, sex, race, sample quality, processing lab and country of residence. Column E provides the log base-2 difference in expression levels for each marker as between asthmatics and healthy volunteers. A positive value indicates higher expression in asthma subjects, a negative value indicates a lower level in asthma subjects. Columns F and G indicate the intra-subject (within subject) variability for each marker within the asthmatic group and the group of healthy volunteers, respectively. Column H indicates the parameters the inventors used in the selection of the gene for inclusion in this biomarker panel.

Table 1b provides the gene symbol for each marker in column A and the average Affymetrix Gene Chip signal for samples derived from the asthmatic group for each marker in Column B. Column C indicates which markers passed or failed the most stringent criteria set used to determine the highest priority markers as described above. Column D provides the p value adjusted for multiplicity of testing using the false discovery rate method when gene expression levels in 1147 samples from 337 asthma subjects were compared to levels in 1183 samples from 348 healthy subjects. ANCOVA was performed to adjust for covariates related to age, sex, race, sample quality, processing lab and country of residence.

Column E of Table 1b indicates, in shorthand form: gene expression that is significantly higher in healthy patients compared to asthmatics (“h”); gene expression that is significantly lower in healthy patients compared to asthmatics (“I”); and gene expression whose difference in expression between healthy patients and asthmatics does not reach a significance threshold of an FDR<0.0001 (“-”). This information is broken down by severity of asthma. Column E uses a three character code, in which the first character represents a comparison of healthy patients to mild asthmatics; the second character represents a comparison of healthy patients to moderate asthmatics; and the third character represents a comparison of healthy patients to severe asthmatics. Thus, for example, the code in column E of Table 1b for CD69 is “-hh”, indicating that CD69 expression is significantly higher in healthy patients than in moderate or severe asthmatics, but that any difference in expression between healthy patients and mild asthmatics does not reach the FDR<0.0001 threshold. In contrast, the code in column E of Table 1b for BASP1 is “III,” indicating that BASP1 expression is significantly lower in healthy patients than in mild, in moderate, and in severe asthmatics.

Columns F and G of Table 1b provide the FDR for each marker in a comparison of marker expression levels in healthy volunteers to asthmatics suffering from moderate and severe forms of asthma, respectively. Column H, I, and J, indicate the absolute fold difference for each marker in a comparison of the expression levels of each in healthy volunteers versus asthmatics with mild, moderate, and severe asthma, respectively. Column K provides the accession numbers for each marker.

Table 6 provides a list of all probe sequences for the markers identified in Tables 1a and b. Each sequence is identified by an Affymetrix qualifier associated with a marker and each marker has multiple probe sequences associated with it.

Of the genes selected by the criteria outlined above, five (5) were determined to be novel, unknown, or not fully characterized, those genes bearing Affymetrix qualifiers 203429_s_at; 210054_at; 222309_at; 212779_at; and 213158_at. Details pertaining to the description of the sequences, aliases, orthologs, and literature citations can be found in Table 2.

Table 2 provides the annotations of the aforementioned previously unknown markers. Columns A and B provide the Affymetrix qualifiers and annotations, respectively, for each marker, if any. Column C indicates any consensus sequences to which the particular probe is similar. Columns D, E, and F provide the National Center for Biotechnology Information (NCBI) gene names, aliases, and gene descriptions, respectively, for each marker, if any. Columns G and H provide the Refseq accession numbers and protein names, respectively, for each marker, if any. Column I indicates any murine or rat orthologs to the markers and Column J provides any transmembrane domain predictions for the markers, including the first and last amino acids in the primary sequence defining the predicted domain. Lastly, Column K provides the gene ontology (GO) annotation for the marker, if any.

Affymetrix qualifier 203429_at is a probe for the 3′ untranslated region of open reading frame (ORF) 9 of chromosome 1 (or C1ORF9). According to the literature, this probe has the alternative name of CH1, or membrane protein CH1. There are at least two (2) variants and the protein's similarity to some orthologs is indicated in column J of Table 2. Variant 1 contains a signal sequence from amino acid 1 to amino acid 29 and a Sad1/UNC-like C-terminal domain. Sad1/UNC from amino acid 322 to amino acid 452 is part of the galactose-binding like superfamily. Variant 2 lacks the signal sequence but bears the Sad1/UNC-like C-terminal domain from amino acid 480 to amino acid 603. The C. elegans UNC-84 protein is a nuclear envelope protein that is involved in nuclear anchoring and migration during development. The S. pombe Sad1 protein localizes at the spindle pole body. UNC-84 and Sad1 share a common C-terminal region that is often termed the SUN (Sad1 and UNC) domain. In mammals, the SUN domain is present in two proteins, Sun1 and Sun2. The SUN domain of Sun2 has been demonstrated to be in the periplasm. The literature reports that membrane protein CH1 has its highest expression in the pancreas and testis with lower levels of expression in the prostate and ovary (Rosok (2000) Biochem. Biophys. Res. Commun. 267(3): 855-862). Rosok also predicts cAMP and cGMP phosphorylation sites in the C-terminal end of the protein and a transmembrane domain (amino acids 1011-1031 of the protein).

Affymetrix qualifier 210054_at is a probe for the 3′ untranslated region of open reading frame 15 of chromosome 4 (C4ORF15) and has alternative names including DKFZp686I1868, IT1, MGC4701, and hypothetical protein LOC79441. The sequence appears to have a similarity to the early endosome antigen Rab effector (EEA1) isoform 1 of Rattus norvegicus.

Affymetrix qualifier 222309_at is a probe for a region in intron 4 of the C6ORF62 (open reading frame 62 in chromosome 6) gene. Expressed sequence tag (EST) evidence indicates that it is a transcribed region. The sequence of intron 4 is provided in Table 8; the shaded region of the sequence represents a portion of intron 4 contiguously connected to the probed region by EST evidence, indicating that at least this region appears to be transcribed. The entire sequence that, based on EST evidence, appears to be transcribed is also provided in Table 8 and is identified as “Transcribed seq.” Thus, this likely constitutes a 3′ UTR of a truncated C6ORF62 gene with a polyadenylation site in the transcribed sequence. Additional sequence, including additional portions of intron 4, may also be present in the detected transcript.

Affymetrix qualifier 212779_at is a probe for the open reading frame and 3′ untranslated region of KIAA1109, which has aliases and gene descriptions DKFZp781P0474, FSA, MCG110967, “fragile site-associated protein,” and hypothetical protein LOC84162. The sequence appears to have similarity (33-39%) with C. elegans proteins q8wtl7_caeel.trembl and q9n3r9_caeel.trembl. Secondary and tertiary protein structure prediction indicates that this protein contains a transmembrane domain (between amino acids 25 and 47) and an aspartate protease domain as well as a coiled coil region between amino acids 96 through 120. It is predicated that this protein is likely an aspartic-type endopeptidase. The literature indicates that elevated FSA mRNA is found in testis and expression of FSA is associated with postmitotic germ cells in spermatogenesis. Enhanced expression of FSA is also observed during adipogenesis in cultured cells. Through bioinformatics analysis, this protein is also reported to contain several nuclear localization signals (i.e., KKLGTALQDEKEKKGKDK, starting at amino acid 2989; KRLWFLWPDDILKNKRCRNK starting at amino acid 523, PKQRRSF starting at amino acid 773, and PGRKKKK starting at amino acid 831) and nuclear export signals (NES) (i.e., LKLPSLDL starting at amino acid 2003, LSGLQL starting at amino acid 304, and LHRPLDL starting at amino acid 947). FSA is a serine-rich protein, with the overall serine content of the polypeptide reaching 11.9% and as high in some stretches (i.e., amino acids 524 to 693) as 28%. Furthermore, the C-terminal portion of FSA shares 21% amino acid sequence similarity to the deduced amino acid sequence encoded by the lipid depleted protein gene (Ipd-3) of C. elegans (NP_{—}491182).

Affymetrix qualifier 213158_at probes for a genomic region with extensive EST support. The ESTs supports a genomic region of 3935 basepairs (bps). There is neither an ORF nor an exon prediction in this region. This sequence appears to probe a long 3′ untranslated region of ZBTB20 (Zinc finger and BTB domain containing 20) (ZBTB20 is located approximately 20 kilobases (kb) upstream of the region being probed by 213158_at). Alternatively, it may probe a non-coding RNA. The 213158_at probe targets a genomic region with extensive EST support that is 23634 bases downstream of ZBTB20. Contiguous EST evidence indicates that the transcript detected by the probes includes the sequence identified as the “transcribed sequence” for 213158_at in Table 8. This is very well conserved in the mouse and again there is EST evidence to support that this region of at least 8439 basepairs is transcribed. The transcribed sequence in the mouse is also provided in Table 8 and identified as “MOUSE TRANSCRIBED SEQ.” Mus ZBTB20 is located approximately 20 kb upstream of the region being probed by 213158_at. In the mouse, there is extensive and, for the most part, overlapping EST evidence in this 23014 bp region to support that ZBTB20 has a very long 3′ UTR. ZBTB belong to the C2H2 zinc finger protein family of transcription factors. The 733-residue long protein contains a BTB/POZ domain at the N-terminal and four (4) C2H2 zinc fingers in the C-terminal. It shares the closest homology to BCL-6, which is widely expressed in hematopoietic tissues, including dendritic cells, monocytes, B cells, and T cells. There is also the possibility of a miRNA prediction in the mouse in this 3′ UTR region approximately 1300 bases upstream of the region probed by 213158_at.

In further studies, approximately 559 genes were determined to be responsive to IL-13 stimulation by the criteria of being called “present” (i.e., Affymetrix Detection p-value<0.04) in at least 25% of the arrays in at least one of twenty-four (24) experimental groups and having a fold-change of >±1.5 at any one or more of four timepoints (timepoints taken at 2 hours, 6 hours, 12 hours, and 24 hours after treatment) with an FDR≦0.05 relative to a time-matched control sample. The complete list of 559 IL-13 responsive genes is given in Table 7.

Table 7 provides the Affymetrix qualifier and gene symbol of the marker of interest in Columns A and B, respectively. Columns C, D, E, and F, provide the FDR for each marker 2 hours, 6 hours, 12 hours, and 24 hours after IL-13 stimulation, respectively. Columns G, H, I, and J indicate the log base-2 fold change in the marker's expression level 2 hours, 6 hours, 12 hours, and 24 hours after IL-13 stimulation, respectively.

As discussed earlier, expression level of markers of the present invention can be used as an indicator of asthma. Expression level of markers of the present invention can also be used as indicators of an IL-13-mediated condition. Detection and measurement of the relative amount of an asthma-associated or IL-13-responsiveness associated marker or marker gene product (polynucleotide or polypeptide) of the invention can be by any method known in the art.

Methodologies for detection of a transcribed polynucleotide can include RNA extraction from a cell or tissue sample, followed by hybridization of a labeled probe (i.e., a complementary polynucleotide molecule) specific for the target RNA to the extracted RNA and detection of the probe (i.e., Northern blotting).

Methodologies for peptide detection include protein extraction from a cell or tissue sample, followed by binding of an antibody specific for the target protein to the protein sample, and detection of the antibody. Antibodies are generally detected by the use of a labeled secondary antibody. The label can be a radioisotope, a fluorescent compound, an enzyme, an enzyme co-factor, or ligand. Such methods are well understood in the art.

Detection of specific polynucleotide molecules may also be assessed by gel electrophoresis, column chromatography, or direct sequencing, quantitative PCR, RT-PCR, or nested PCR among many other techniques well known to those skilled in the art.

Detection of the presence or number of copies of all or part of a marker as defined by the invention may be performed using any method known in the art. It is convenient to assess the presence and/or quantity of a DNA or cDNA by Southern analysis, in which total DNA from a cell or tissue sample is extracted, is hybridized with a labeled probe (i.e., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as would be understood by one skilled in the art.

### Diagnosis, Prognosis, and Assessment of Asthma and IL-13-Mediated Conditions

The asthma markers and IL-13 responsive markers disclosed in the present invention can be employed in diagnostic methods comprising the steps of (a) detecting an expression level of such a marker in a patient; (b) comparing that expression level to a reference expression level of the same marker; (c) and diagnosing a patient has having or not having asthma, or an IL-13-mediated condition based upon the comparison made. The methods described herein below, including preparation of blood and other tissue samples, assembly of class predictors, and construction and comparison of expression profiles, can be readily adapted for the diagnosis of, assessment of, and selection of a treatment for asthma and IL-13-mediated conditions. This can be achieved by comparing the expression profile of one or more of the markers in a subject of interest to at least one reference expression profile of the markers. The reference expression profile(s) can include an average expression profile or a set of individual expression profiles each of which represents the gene expression of the asthma or IL-13 responsive markers in a particular asthma patient, a patient with an IL-13-mediated condition, or disease-free human. Similarity between the expression profile of the subject of interest and the reference expression profile(s) is indicative of the presence or absence of the disease state of asthma or the IL-13-mediated condition. In many embodiments, the disease genes employed for the diagnosis or monitoring of asthma or the IL-13-mediated condition are selected from the markers described in Table 1a and b. In some embodiments, the disease genes employed for the diagnosis or monitoring of asthma or the IL-13-mediated condition are selected from the markers in Table 1b wherein “yes” is indicated in Column C. One or more asthma or IL-13 responsive markers selected from Table 1a and b can be used for asthma or IL-13-mediated condition diagnosis or disease monitoring. In one embodiment, each marker has a p-value of less than 0.01, 0.005, 0.001, 0.0005, 0.0001, or less. In another embodiment, the asthma genes/markers comprise at least one gene having an “Asthma/Disease-Free” ratio of no less than 2 and at least one gene having an “Asthma/Disease-Free” ratio of no more than 0.5. In a further embodiment, the IL-13 responsive genes/markers comprise at least one gene having an “IL-13-mediated Condition/Condition-Free” ratio of no less than 2 and at least one gene having an “IL-13-mediated Condition/Condition-Free” ratio of no more than 0.5. A diagnosis of a patient as having asthma or an IL-13-mediated condition can be established under a range of ratios, wherein a significant difference can be ratio of the marker expression level to healthy expression level of the marker of >|1| (absolute value of 1). Such significantly different ratios can include, but are not limited to, the absolute values of 1.001, 1.01, 1.05, 1.1, 1.2, 1.3, 1.5, 1.7, 2, 3, 4, 5, 6, 7, 10, or any and all ratios commonly understood to be significant by the skilled practitioner.

The asthma and IL-13 responsive markers of the present invention can be used alone, or in combination with other clinical tests, for asthma or IL-13-mediated condition diagnosis or disease monitoring. Conventional methods for detecting or diagnosing asthma or IL-13-mediated conditions include, but are not limited to, blood tests, chest X-ray, biopsies, skin tests, mucus tests, urine/excreta sample testing, physical exam, or any and all related clinical examinations known to the skilled artisan. Any of these methods, as well as any other conventional or non-conventional method, can be used, in addition to the methods of the present invention, to improve the accuracy of the diagnosis or monitoring of asthma or an IL-13-mediated condition.

The markers of the present invention can also be used for the determination or assessment of the severity of a patient's asthma. In particular, the present invention provides markers, the upregulation or downregulation of which is indicative of mild, moderate, or severe asthma. The capacity for a given marker to provide a determination or assessment of asthma severity is provided in Table 1b, Column E.

The markers of the present invention can also be used for the prediction of the clinical outcome, or prognosis, of an asthma or IL-13-mediated condition patient of interest. The prediction typically involves comparison of the peripheral blood expression profile, or expression profile from another tissue, of one or more markers in the patient of interest to at least one reference expression profile. Each marker employed in the present invention is differentially expressed in peripheral blood samples, or other tissue samples, of asthma or IL-13-mediated condition patients who have different clinical outcomes.

In one embodiment, the markers employed for providing a diagnosis are selected such that the peripheral blood expression profile of each marker is correlated with a class distinction under a class-based correlation analysis (such as the nearest-neighbor analysis), where the class distinction represents an idealized expression pattern of the selected genes in tissue samples, such as peripheral blood samples, of asthma or IL-13-mediated condition patients and healthy volunteers. In many cases, the selected markers are correlated with the class distinction at above the 50%, 25%, 10%, 5%, or 1% significance level under a random permutation test.

In one embodiment, the markers employed for providing a prognosis are selected such that the peripheral blood expression profile of each marker is correlated with a class distinction under a class-based correlation analysis (such as the nearest-neighbor analysis), where the class distinction represents an idealized expression pattern of the selected genes in tissue samples, such as peripheral blood samples, of asthma or IL-13-mediated condition patients who have different clinical outcomes. In many cases, the selected markers are correlated with the class distinction at above the 50%, 25%, 10%, 5%, or 1% significance level under a random permutation test.

The markers can also be selected such that the average expression profile of each marker in tissue samples, such as peripheral blood samples, of one class of asthma or IL-13-mediated condition patients is statistically different from that in another class of patients. For instance, the p-value under a Student's t-test for the observed difference can be no more than 0.05, 0.01, 0.005, 0.001, or less. In addition, the markers can be selected such that the average expression level of each marker in one class of patients is at least 2-, 3-, 4-, 5-, 10-, or 20-fold different from that in another class of patients.

The expression profile of a patient of interest can be compared to one or more reference expression profiles. The reference expression profiles can be determined concurrently with the expression profile of the patient of interest. The reference expression profiles can also be predetermined or prerecorded in electronic or other types of storage media.

The reference expression profiles can include average expression profiles, or individual profiles representing gene expression patterns in particular patients. In one embodiment, the reference expression profiles used for a diagnosis of asthma or an IL-13-mediated condition include an average expression profile of the marker(s) in tissue samples, such as peripheral blood samples, of healthy volunteers. In one embodiment, the reference expression profiles include an average expression profile of the marker(s) in tissue samples, such as peripheral blood samples, of reference patients who have known or determinable disease status or clinical outcomes. Any averaging method may be used, such as arithmetic means, harmonic means, average of absolute values, average of log-transformed values, or weighted average. In one example, the reference asthma patients or IL-13-mediated condition patients have the same disease status or clinical outcome. In another example, the reference patients can are healthy volunteers used in a diagnostic method. In another example, the reference patients can be divided into at least two classes, each class of patients having a different respective disease status or clinical outcome. The average expression profile in each class of patients constitutes a separate reference expression profile, and the expression profile of the patient of interest is compared to each of these reference expression profiles.

In another embodiment, the reference expression profiles include a plurality of expression profiles, each of which represents the expression pattern of the marker(s) in a particular asthma patient or IL-13-mediated condition patient. Other types of reference expression profiles can also be used in the present invention. In yet another embodiment, the present invention uses a numerical threshold as a control level. The numerical threshold may comprise a ratio, including, but not limited to, the ratio of the expression level of a marker in a patient in relation to the expression level of the same marker in a healthy volunteer; or the ratio between the expression levels of the marker in a patient both before and after treatment. The numerical threshold may also by a ratio of marker expression levels between patients with differing disease status or clinical outcomes.

In another embodiment, the absolute expression level(s) of the marker(s) are detected or measured and compared to reference expression level(s) for the purposes of providing a diagnosis or aiding in the selection of a treatment. The reference expression level is obtained from a control sample in this embodiment, the control sample being derived from either a healthy individual or an asthma or IL-13-mediated condition patient prior to treatment.

The expression profile of the patient of interest and the reference expression profile(s) can be constructed in any form. In one embodiment, the expression profiles comprise the expression level of each marker used in outcome prediction. The expression levels can be absolute, normalized, or relative levels. Suitable normalization procedures include, but are not limited to, those used in nucleic acid array gene expression analyses or those described in Hill, et al., GENOME BIOL., 2:research0055.1-0055.13 (2001). In one example, the expression levels are normalized such that the mean is zero and the standard deviation is one. In another example, the expression levels are normalized based on internal or external controls, as appreciated by those skilled in the art. In still another example, the expression levels are normalized against one or more control transcripts with known abundances in blood samples. In many cases, the expression profile of the patient of interest and the reference expression profile(s) are constructed using the same or comparable methodologies.

In another embodiment, each expression profile being compared comprises one or more ratios between the expression levels of different markers. An expression profile can also include other measures that are capable of representing gene expression patterns.

The peripheral blood samples used in the present invention can be either whole blood samples, or samples comprising enriched PBMCs. In one example, the peripheral blood samples used for preparing the reference expression profile(s) comprise enriched or purified PBMCs, and the peripheral blood sample used for preparing the expression profile of the patient of interest is a whole blood sample. In another example, all of the peripheral blood samples employed in outcome prediction comprise enriched or purified PBMCs. In many cases, the peripheral blood samples are prepared from the patient of interest and reference patients using the same or comparable procedures.

Other types of blood samples can also be employed in the present invention, and the gene expression profiles in these blood samples are statistically significantly correlated with patient outcome.

The blood samples used in the present invention can be isolated from respective patients at any disease or treatment stage, and the correlation between the gene expression patterns in these blood samples, the health status, or clinical outcome is statistically significant. In many embodiments, the health status is measured by a comparison of the patient's expression profile or absolute marker(s) expression level(s) as compared to an absolute level of a marker in one or more healthy volunteers or an averaged or correlated expression profile from two or more healthy volunteers. In many embodiments, clinical outcome is measured by patients' response to a therapeutic treatment, and all of the blood samples used in outcome prediction are isolated prior to the therapeutic treatment. The expression profiles derived from the blood samples are therefore baseline expression profiles for the therapeutic treatment.

Construction of the expression profiles typically involves detection of the expression level of each marker used in the health status determination or outcome prediction. Numerous methods are available for this purpose. For instance, the expression level of a gene can be determined by measuring the level of the RNA transcript(s) of the gene(s). Suitable methods include, but are not limited to, quantitative RT-PCR, Northern blot, in situ hybridization, slot-blotting, nuclease protection assay, and nucleic acid array (including bead array). The expression level of a gene can also be determined by measuring the level of the polypeptide(s) encoded by the gene. Suitable methods include, but are not limited to, immunoassays (such as ELISA, RIA, FACS, or Western blot), 2-dimensional gel electrophoresis, mass spectrometry, or protein arrays.

In one aspect, the expression level of a marker is determined by measuring the RNA transcript level of the gene in a tissue sample, such as a peripheral blood sample. RNA can be isolated from the peripheral blood or tissue sample using a variety of methods. Exemplary methods include guanidine isothiocyanate/acidic phenol method, the TRIZOL® Reagent (Invitrogen), or the Micro-FastTrack™ 2.0 or FastTrack™ 2.0 mRNA Isolation Kits (Invitrogen). The isolated RNA can be either total RNA or mRNA. The isolated RNA can be amplified to cDNA or cRNA before subsequent detection or quantitation. The amplification can be either specific or non-specific. Suitable amplification methods include, but are not limited to, reverse transcriptase PCR (RT-PCR), isothermal amplification, ligase chain reaction, and Qbeta replicase.

In one embodiment, the amplification protocol employs reverse transcriptase. The isolated mRNA can be reverse transcribed into cDNA using a reverse transcriptase, and a primer consisting of oligo (dT) and a sequence encoding the phage T7 promoter. The cDNA thus produced is single-stranded. The second strand of the cDNA is synthesized using a DNA polymerase, combined with an RNase to break up the DNA/RNA hybrid. After synthesis of the double-stranded cDNA, T7 RNA polymerase is added, and cRNA is then transcribed from the second strand of the doubled-stranded cDNA. The amplified cDNA or cRNA can be detected or quantitated by hybridization to labeled probes. The cDNA or cRNA can also be labeled during the amplification process and then detected or quantitated.

In another embodiment, quantitative RT-PCR (such as TaqMan, ABI) is used for detecting or comparing the RNA transcript level of a marker of interest. Quantitative RT-PCR involves reverse transcription (RT) of RNA to cDNA followed by relative quantitative PCR (RT-PCR).

In PCR, the number of molecules of the amplified target DNA increases by a factor approaching two with every cycle of the reaction until some reagent becomes limiting. Thereafter, the rate of amplification becomes increasingly diminished until there is not an increase in the amplified target between cycles. If a graph is plotted on which the cycle number is on the X axis and the log of the concentration of the amplified target DNA is on the Y axis, a curved line of characteristic shape can be formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR is proportional to the starting concentration of the target before the PCR is begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction.

The final concentration of the target DNA in the plateau portion of the curve is determined by the availability of reagents in the reaction mix and is independent of the original concentration of target DNA. Therefore, in one embodiment, the sampling and quantifying of the amplified PCR products are carried out when the PCR reactions are in the linear portion of their curves. In addition, relative concentrations of the amplifiable cDNAs can be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes internal PCR standards that are approximately as abundant as the target. This strategy is effective if the products of the PCR amplifications are sampled during their linear phases. If the products are sampled when the reactions are approaching the plateau phase, then the less abundant product may become relatively over-represented. Comparisons of relative abundances made for many different RNA samples, such as is the case when examining RNA samples for differential expression, may become distorted in such a way as to make differences in relative abundances of RNAs appear less than they actually are. This can be improved if the internal standard is much more abundant than the target. If the internal standard is more abundant than the target, then direct linear comparisons may be made between RNA samples.

A problem inherent in clinical samples is that they are of variable quantity or quality. This problem can be overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs. While empirical determination of the linear range of the amplification curve and normalization of cDNA preparations are tedious and time-consuming processes, the resulting RT-PCR assays may, in certain cases, be superior to those derived from a relative quantitative RT-PCR with an internal standard.

In yet another embodiment, nucleic acid arrays (including bead arrays) are used for detecting or comparing the expression profiles of a marker of interest. The nucleic acid arrays can be commercial oligonucleotide or cDNA arrays. They can also be custom arrays comprising concentrated probes for the markers of the present invention. In many examples, at least 15%, 20%, 25%, 30%, 35%, 40%, 45%, 50%, or more of the total probes on a custom array of the present invention are probes for asthma markers. These probes can hybridize under stringent or nucleic acid array hybridization conditions to the RNA transcripts, or the complements thereof, of the corresponding markers.

As used herein, “stringent conditions” are at least as stringent as, for example, conditions G-L shown in Table 5. “Highly stringent conditions” are at least as stringent as conditions A-F shown in Table 5. Hybridization is carried out under the hybridization conditions (Hybridization Temperature and Buffer) for about four hours, followed by two 20-minute washes under the corresponding wash conditions (Wash Temp and Buffer).

In one example, a nucleic acid array of the present invention includes at least 2, 5, 10, or more different probes. Each of these probes is capable of hybridizing under stringent or nucleic acid array hybridization conditions to a different respective marker of the present invention. Multiple probes for the same marker can be used on the same nucleic acid array. The probe density on the array can be in any range.

The probes for a marker of the present invention can be a nucleic acid probe, such as, DNA, RNA, PNA, or a modified form thereof. The nucleotide residues in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

The probes for the markers can be stably attached to discrete regions on a nucleic acid array. By “stably attached,” it means that a probe maintains its position relative to the attached discrete region during hybridization and signal detection. The position of each discrete region on the nucleic acid array can be either known or determinable. All of the methods known in the art can be used to make the nucleic acid arrays of the present invention.

In another embodiment, nuclease protection assays are used to quantitate RNA transcript levels in peripheral blood samples. There are many different versions of nuclease protection assays. The common characteristic of these nuclease protection assays is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. Examples of suitable nuclease protection assays include the RNase protection assay provided by Ambion, Inc. (Austin, Tex.).

Hybridization probes or amplification primers for the markers of the present invention can be prepared by using any method known in the art.

In one embodiment, the probes/primers for a marker significantly diverge from the sequences of other markers. This can be achieved by checking potential probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. The initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by those skilled in the art.

In another embodiment, the probes for markers can be polypeptide in nature, such as, antibody probes. The expression levels of the markers of the present invention are thus determined by measuring the levels of polypeptides encoded by the markers. Methods suitable for this purpose include, but are not limited to, immunoassays such as ELISA, RIA, FACS, dot blot, Western Blot, immunohistochemistry, and antibody-based radio-imaging. In addition, high-throughput protein sequencing, 2-dimensional SDS-polyacrylamide gel electrophoresis, mass spectrometry, or protein arrays can be used.

In one embodiment, ELISAs are used for detecting the levels of the target proteins. In an exemplifying ELISA, antibodies capable of binding to the target proteins are immobilized onto selected surfaces exhibiting protein affinity, such as wells in a polystyrene or polyvinylchloride microtiter plate. Samples to be tested are then added to the wells. After binding and washing to remove non-specifically bound immunocomplexes, the bound antigen(s) can be detected. Detection can be achieved by the addition of a second antibody which is specific for the target proteins and is linked to a detectable label. Detection can also be achieved by the addition of a second antibody, followed by the addition of a third antibody that has binding affinity for the second antibody, with the third antibody being linked to a detectable label. Before being added to the microtiter plate, cells in the samples can be lysed or extracted to separate the target proteins from potentially interfering substances.

In another exemplifying ELISA, the samples suspected of containing the target proteins are immobilized onto the well surface and then contacted with the antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another exemplary ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can be used. After binding of a protein or antibody to the well, coating with a non-reactive material to reduce background, and washing to remove unbound material, the immobilizing surface is contacted with the control or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex is facilitated by using a labeled secondary binding ligand or antibody, or a secondary binding ligand or antibody in conjunction with a labeled tertiary antibody or third binding ligand.

Following all incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent washing to remove unbound material, the amount of label can be quantified, e.g., by incubation with a chromogenic substrate such as urea and bromocresol purple or 2,2′-azido-di-(3-ethyl)-benzthiazoline-6-sulfonic acid (ABTS) and H₂O₂, in the case of peroxidase as the enzyme label. Quantitation can be achieved by measuring the degree of color generation, e.g., using a spectrophotometer.

Another method suitable for detecting polypeptide levels is RIA (radioimmunoassay). An exemplary RIA is based on the competition between radiolabeled-polypeptides and unlabeled polypeptides for binding to a limited quantity of antibodies. Suitable radiolabels include, but are not limited to, I¹²⁵. In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this standard curve, the concentration of the polypeptide in unknown samples can be determined. Protocols for conducting RIA are well known in the art.

Suitable antibodies for the present invention include, but are not limited to, polyclonal antibodies, monoclonal antibodies, chimeric antibodies, humanized antibodies, single chain antibodies, Fab fragments, or fragments produced by a Fab expression library. Neutralizing antibodies (i.e., those which inhibit dimer formation) can also be used. Methods for preparing these antibodies are well known in the art. In one embodiment, the antibodies of the present invention can bind to the corresponding marker gene products or other desired antigens with binding affinities of at least 10⁴ M⁻¹, 10⁵ M⁻¹, 10⁶ M⁻¹, 10⁷ M⁻¹, or more.

The antibodies of the present invention can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

The antibodies of the present invention can be used as probes to construct protein arrays for the detection of expression profiles of the markers. Methods for making protein arrays or biochips are well known in the art. In many embodiments, a substantial portion of probes on a protein array of the present invention are antibodies specific for the marker products. For instance, at least 10%, 20%, 30%, 40%, 50%, or more probes on the protein array can be antibodies specific for the marker gene products.

In yet another aspect, the expression levels of the markers are determined by measuring the biological functions or activities of these genes. Where a biological function or activity of a gene is known, suitable in vitro or in vivo assays can be developed to evaluate the function or activity. These assays can be subsequently used to assess the level of expression of the marker.

After the expression level of each marker is determined, numerous approaches can be employed to compare expression profiles. Comparison of the expression profile of a patient of interest to the reference expression profile(s) can be conducted manually or electronically. In one example, comparison is carried out by comparing each component in one expression profile to the corresponding component in a reference expression profile. The component can be the expression level of a marker, a ratio between the expression levels of two markers, or another measure capable of representing gene expression patterns. The expression level of a gene can have an absolute or a normalized or relative value. The difference between two corresponding components can be assessed by fold changes, absolute differences, or other suitable means.

Comparison of the expression profile of a patient of interest to the reference expression profile(s) can also be conducted using pattern recognition or comparison programs, such as the k-nearest-neighbors algorithm as described in Armstrong (Armstrong (2002) Nature Genetics, 30:4147), or the weighted voting algorithm as described below. In addition, the serial analysis of gene expression (SAGE) technology, the GEMTOOLS gene expression analysis program (Incyte Pharmaceuticals), the GeneCalling and Quantitative Expression Analysis technology (Curagen), and other suitable methods, programs or systems can be used to compare expression profiles.

Multiple markers can be used in the comparison of expression profiles. For instance, 2, 4, 6, 8, 10, 12, 14, or more markers can be used. In addition, the marker(s) used in the comparison can be selected to have relatively small p-values (e.g., two-sided p-values). In many examples, the p-values indicate the statistical significance of the difference between gene expression levels in different classes of patients. In many other examples, the p-values suggest the statistical significance of the correlation between gene expression patterns and clinical outcome. In one embodiment, the markers used in the comparison have p-values of no greater than 0.05, 0.01, 0.001, 0.0005, 0.0001, or less. Markers with p-values of greater than 0.05 can also be used. These genes may be identified, for instance, by using a relatively small number of blood samples.

Similarity or difference between the expression profile of a patient of interest and a reference expression profile is indicative of the class membership of the patient of interest. Similarity or difference can be determined by any suitable means. The comparison can be qualitative, quantitative, or both.

In one example, a component in a reference profile is a mean value, and the corresponding component in the expression profile of the patient of interest falls within the standard deviation of the mean value. In such a case, the expression profile of the patient of interest may be considered similar to the reference profile with respect to that particular component. Other criteria, such as a multiple or fraction of the standard deviation or a certain degree of percentage increase or decrease, can be used to measure similarity.

In another example, at least 50% (e.g., at least 60%, 70%, 80%, 90%, or more) of the components in the expression profile of the patient of interest are considered similar to the corresponding components in a reference profile. Under these circumstances, the expression profile of the patient of interest may be considered similar to the reference profile. Different components in the expression profile may have different weights for the comparison. In some cases, lower percentage thresholds (e.g., less than 50% of the total components) are used to determine similarity.

The marker(s) and the similarity criteria can be selected such that the accuracy of the diagnostic determination or the outcome prediction (the ratio of correct calls over the total of correct and incorrect calls) is relatively high. For instance, the accuracy of the determination or prediction can be at least 50%, 60%, 70%, 80%, 90%, or more.

The effectiveness of treatment prediction can also be assessed by sensitivity and specificity. The markers and the comparison criteria can be selected such that both the sensitivity and specificity of outcome prediction are relatively high. For instance, the sensitivity and specificity can be at least 50%, 60%, 70%, 80%, 90%, 95%, or more. As used herein, “sensitivity” refers to the ratio of correct positive calls over the total of true positive calls plus false negative calls, and “specificity” refers to the ratio of correct negative calls over the total of true negative calls plus false positive calls.

Moreover, peripheral blood expression profile-based health status determination or outcome prediction can be combined with other clinical evidence to aid in treatment selection, improve the effectiveness of treatment, or accuracy of outcome prediction.

In many embodiments, the expression profile of a patient of interest is compared to at least two reference expression profiles. Each reference expression profile can include an average expression profile, or a set of individual expression profiles each of which represents the gene expression pattern in a particular asthma patient or disease-free human. Suitable methods for comparing one expression profile to two or more reference expression profiles include, but are not limited to, the weighted voting algorithm or the k-nearest-neighbors algorithm. Softwares capable of performing these algorithms include, but are not limited to, GeneCluster 2 software. GeneCluster2 software is available from MIT Center for Genome Research at Whitehead Institute. Both the weighted voting and k-nearest-neighbors algorithms employ gene classifiers that can effectively assign a patient of interest to a health status, outcome or effectiveness of treatment class. By “effectively,” it means that the class assignment is statistically significant. In one example, the effectiveness of class assignment is evaluated by leave-one-out cross validation or k-fold cross validation. The prediction accuracy under these cross validation methods can be, for instance, at least 50%, 60%, 70%, 80%, 90%, 95%, or more. The prediction sensitivity or specificity under these cross validation methods can also be at least 50%, 60%, 70%, 80%, 90%, 95%, or more. Markers or class predictors with low assignment sensitivity/specificity or low cross validation accuracy, such as less than 50%, can also be used in the present invention.

Under one version of the weighted voting algorithm, each gene in a class predictor casts a weighted vote for one of the two classes (class 0 and class 1). The vote of gene “g” can be defined as v_{g}=a_{g} (x_{g}−b_{g}), wherein a_{g} equals to P(g,c) and reflects the correlation between the expression level of gene “g” and the class distinction between the two classes, b_{g} is calculated as b_{g}=[x0(g)+x1(g)]/2 and represents the average of the mean logs of the expression levels of gene “g” in class 0 and class 1, and x_{g} is the normalized log of the expression level of gene “g” in the sample of interest. A positive v_{g} indicates a vote for class 0, and a negative v_{g} indicates a vote for class 1. V0 denotes the sum of all positive votes, and V1 denotes the absolute value of the sum of all negative votes. A prediction strength PS is defined as PS=(V0−V1)/(V0+V1). Thus, the prediction strength varies between −1 and 1 and can indicate the support for one class (e.g., positive PS) or the other (e.g., negative PS). A prediction strength near “0” suggests narrow margin of victory, and a prediction strength close to “1” or “−1” indicates wide margin of victory. See Slonim (2000) Procs. of the Fourth Annual International Conference on Computational Molecular Biology, Tokyo, Japan, April 8-11, p 263-272; and Golub (1999) Science, 286: 531-537.

Suitable prediction strength (PS) thresholds can be assessed by plotting the cumulative cross-validation error rate against the prediction strength. In one embodiment, a positive predication is made if the absolute value of PS for the sample of interest is no less than 0.3. Other PS thresholds, such as no less than 0.1, 0.2, 0.4 or 0.5, can also be selected for class prediction. In many embodiments, a threshold is selected such that the accuracy of prediction is optimized and the incidence of both false positive and false negative results is minimized.

Any class predictor constructed according to the present invention can be used for the class assignment of an asthma or IL-13-mediated condition patient of interest. In many examples, a class predictor employed in the present invention includes n markers identified by the neighborhood analysis, where n is an integer greater than 1.

The expression profile of a patient of interest can also be compared to two or more reference expression profiles by other means. For instance, the reference expression profiles can include an average peripheral blood expression profile for each class of patients. The fact that the expression profile of a patient of interest is more similar to one reference profile than to another suggests that the patient of interest is more likely to have the clinical outcome associated with the former reference profile than that associated with the latter reference profile.

In another embodiment, average expression profiles can be compared to each other as well as to a reference expression profile. In one embodiment, an expression profile of a patient is compared to a reference expression profile derived from a healthy volunteer or healthy volunteers, and is also compared to an expression profile of an asthma patient or patients to make a diagnosis. In another embodiment, an expression profile of an asthma patient before treatment is compared to a reference expression profile, and is also compared to an expression profile of the same asthma or IL-13-mediated condition patient after treatment to determine the effectiveness of the treatment. In another embodiment, the expression profiles of the patient both before and after treatment are compared to a reference expression profile, as well as to each other.

In one particular embodiment, the present invention features diagnosis of a patient of interest. Patients can be divided into two classes based on their over- and/or under-expression of asthma or IL-13-responsive markers of interest. One class of patients is diagnosed as having asthma or an IL-13-mediated condition and the other does not (healthy volunteers). Asthma or IL-13 responsive markers that are correlated with a class distinction between those two classes of patients can be identified and then used to assign the patient of interest to one of these two health status classes, thus rendering a diagnosis. Examples of asthma and IL-13 responsive markers suitable for this purpose are depicted in Table 1a and b. In some embodiments, the markers used may be selected from the markers in Table 1b wherein “yes” is indicated in Column C.

In one particular embodiment, the present invention features prediction of clinical outcome or prognosis of an asthma or IL-13-mediated condition patient of interest. Asthma or IL-13-mediated condition patients can be divided into at least two classes based on their responses to a specified treatment regimen. One class of patients (responders) has complete relief of symptoms in response to the treatment, and the other class of patients (non-responders) has neither complete relief from the symptoms nor partial relief in response to the treatment. Asthma or IL-13 responsive markers that are correlated with a class distinction between those two classes of patients can be identified and then used to assign the patient of interest to one of these two outcome classes. Examples of asthma and IL-13 responsive markers suitable for this purpose are depicted in Table 1a and b. In some embodiments, the markers used may be selected from the markers in Table 1b wherein “yes” is indicated in Column C.

The present invention also provides for a method for selecting a treatment or treatment regime involving the use of one or more of the markers of the invention in the diagnosis of the patient as previously described. In a particular embodiment, the expression level of one or more markers of the present invention can be detected and compared to a reference expression level with the subsequent diagnosis of the patient as having asthma or an IL-13-mediated condition should the comparison indicate as such. If the patient is diagnosed as having asthma or an IL-13-mediated condition, treatments or treatment regimes known in the art may be applied in conjunction with this method. Diagnosis of the patient may be determined using any and all of the methods described relating to comparative and statistical methods, techniques, and analyses of marker expression levels, as well as any and all such comparative and statistical methods, techniques, and analyses known to, and commonly used by, one skilled in the art of pharmacogenomics.

In one example, the treatment or treatment regime includes the administration of at least one therapeutic selected from the group including, but not limited to, an IL-13 antagonist, an IL-13 antibody, an anti-histamine, a steroid, an immunomodulator, an IgE downregulator, an immunosuppressant, a bronchodilator/beta-2 agonist, an adenosine A2a receptor agonist, a leukotriene antagonist, a thromboxane A2 synthesis inhibitor, a 5-lipoxygenase inhibitor, an anti-cholinergic, a LTB-4 antagonist, a K+ channel opener, a VLA-4 antagonist, a neurokine antagonist, theophylline, a thromboxane A2 receptor antagonist, a beta-2 adrenoceptor agonist, a soluble interleukin receptor, a 5-lipoxygenase activating protein inhibitor, an arachidonic acid antagonist, an anti-inflammatory, a membrane channel inhibitor, an anti-interleukin antibody, a PDE-4 inhibitor, and a protease inhibitor. Treatments or treatment regimes may also include, but are not limited to, drug therapy, including any and all treatments/therapeutics exemplified in Tables 3 and 4, gene therapy, immunotherapy, radiation therapy, biological therapy, and surgery, as well as any and all other therapeutic methods and treatments known to, and commonly used by, the skilled artisan.

Markers or class predictors capable of distinguishing three or more outcome classes can also be employed in the present invention. These markers can be identified using multi-class correlation metrics. Suitable programs for carrying out multi-class correlation analysis include, but are not limited to, GeneCluster 2 software (MIT Center for Genome Research at Whitehead Institute, Cambridge, Mass.). Under the analysis, patients having asthma or an IL-13-mediated condition are divided into at least three classes, and each class of patients has a different respective clinical outcome. The markers identified under multi-class correlation analysis are differentially expressed in one embodiment in PBMCs of one class of patients relative to PBMCs of other classes of patients. In one embodiment, the identified markers are correlated with a class distinction at above the 1%, 5%, 10%, 25%, or 50% significance level under a permutation test. The class distinction in this embodiment represents an idealized expression pattern of the identified genes in peripheral blood samples of patients who have different clinical outcomes.

### Gene Expression Analysis

The relationship between tissue gene expression profiles, especially peripheral blood gene expression profiles, and diagnosis, prognosis, treatment selection, or treatment effectiveness can be evaluated by using global gene expression analyses. Methods suitable for this purpose include, but are not limited to, nucleic acid arrays (such as cDNA or oligonucleotide arrays), 2-dimensional SDS-polyacrylamide gel electrophoresis/mass spectrometry, and other high throughput nucleotide or polypeptide detection techniques.

Nucleic acid arrays allow for quantitative detection of the expression of a large number of genes at one time. Examples of nucleic acid arrays include, but are not limited to, Genechip® microarrays from Affymetrix (Santa Clara, Calif.), cDNA microarrays from Agilent Technologies (Palo Alto, Calif.), and bead arrays described in U.S. Pat. Nos. 6,228,220, and 6,391,562.

The polynucleotides to be hybridized to a nucleic acid array can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical, or chemical means. Exemplary labeling moieties include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors, and acceptors, and the like. Unlabeled polynucleotides can also be employed. The polynucleotides can be DNA, RNA, or a modified form thereof.

Hybridization reactions can be performed in absolute or differential hybridization formats. In the absolute hybridization format, polynucleotides derived from one sample, such as PBMCs from a patient in a selected health status or outcome class, are hybridized to the probes on a nucleic acid array. Signals detected after the formation of hybridization complexes correlate to the polynucleotide levels in the sample. In the differential hybridization format, polynucleotides derived from two biological samples, such as one from a patient in a first status or outcome class and the other from a patient in a second status or outcome class, are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

Signals gathered from a nucleic acid array can be analyzed using commercially available software, such as those provided by Affymetrix or Agilent Technologies. Controls, such as for scan sensitivity, probe labeling, and cDNA/cRNA quantitation, can be included in the hybridization experiments. In many embodiments, the nucleic acid array expression signals are scaled or normalized before being subject to further analysis. For instance, the expression signals for each gene can be normalized to take into account variations in hybridization intensities when more than one array is used under similar test conditions. Signals for individual polynucleotide complex hybridization can also be normalized using the intensities derived from internal normalization controls contained on each array. In addition, genes with relatively consistent expression levels across the samples can be used to normalize the expression levels of other genes. In one embodiment, the expression levels of genes are normalized across the samples such that the mean is zero and the standard deviation is one. In another embodiment, the expression data detected by nucleic acid arrays are subject to a variation filter that excludes genes showing minimal or insignificant variation across all samples.

### Correlation Analysis

The gene expression data collected from nucleic acid arrays can be correlated with diagnosis, clinical outcome, treatment selection, or treatment effectiveness using a variety of methods. Methods suitable for this purpose include, but are not limited to, statistical methods (such as Spearman's rank correlation, Cox proportional hazard regression model, ANOVA/t test, or other rank tests or survival models) and class-based correlation metrics (such as nearest-neighbor analysis).

In one embodiment, patients with asthma are divided into at least two classes based on their responses to a therapeutic treatment. In another embodiment, a patient of interest can be determined to belong to one of two classes based on the patient's health status. The correlation between peripheral blood gene expression (e.g., PBMC gene expression) and the health status, patient outcome or treatment effectiveness classes is then analyzed by a supervised cluster or learning algorithm. Supervised algorithms suitable for this purpose include, but are not limited to, nearest-neighbor analysis, support vector machines, the SAM method, artificial neural networks, and SPLASH. Under a supervised analysis, health status or clinical outcome of, or treatment effectiveness for, each patient is either known or determinable. Genes that are differentially expressed in peripheral blood cells (e.g., PBMCs) of one class of patients relative to another class of patients can be identified. These genes can be used as surrogate markers for predicting/determining health status or clinical outcome of, or treatment effectiveness for, an asthma or IL-13-mediated condition patient of interest. Many of the genes thus identified are correlated with a class distinction that represents an idealized expression pattern of these genes in patients of different health status, outcome, or treatment effectiveness classes.

In another embodiment, patients with asthma or an IL-13-mediated condition can be divided into at least two classes based on their peripheral blood gene expression profiles. Methods suitable for this purpose include unsupervised clustering algorithms, such as self-organized maps (SOMs), k-means, principal component analysis, and hierarchical clustering. A substantial number (e.g., at least 50%, 60%, 70%, 80%, 90%, or more) of patients in one class may have a first health status, clinical outcome, or treatment effectiveness profile, and a substantial number of patient in another class my have a second health status, clinical outcome, or treatment effectiveness profile. Genes that are differentially expressed in the peripheral blood cells of one class of patients relative to another class of patients can be identified. These genes can also be used as markers for predicting/determining health status, clinical outcome of, or treatment effectiveness for, an asthma or IL-13-mediated condition patient of interest.

In yet another embodiment, patients with asthma or an IL-13-mediated condition can be divided into three or more classes based on their clinical outcomes or peripheral blood gene expression profiles. Multi-class correlation metrics can be employed to identify genes that are differentially expressed in one class of patients relative to another class. Exemplary multi-class correlation metrics include, but are not limited to, those employed by GeneCluster 2 software provided by MIT Center for Genome Research at Whitehead Institute (Cambridge, Mass.).

In a further embodiment, nearest-neighbor analysis (also known as neighborhood analysis) is used to correlate peripheral blood gene expression profiles with health status, clinical outcome of, or treatment effectiveness for, asthma or IL-13-mediated condition patients. The algorithm for neighborhood analysis is described in Slonim (2000) Procs. of the Fourth Annual International Conference on Computational Molecular Biology, Tokyo, Japan, April 8-11, p 263-272; Golub (1999) Science, 286: 531-537; and U.S. Pat. No. 6,647,341. Under one version of the neighborhood analysis, the expression profile of each gene can be represented by an expression vector g=(e₁, e₂, e₃, . . . , eₙ), where eᵢ corresponds to the expression level of gene “g” in the ith sample. A class distinction can be represented by an idealized expression pattern c=(c₁, c₂, c₃, . . . , cₙ), where cᵢ=1 or −1, depending on whether the ith sample is isolated from class 0 or class 1. Class 0 may include patients having a first health status, clinical outcome, or treatment effectiveness profile, and class 1 includes patients having a second health status, clinical outcome, or treatment effectiveness profile. Other forms of class distinction can also be employed. Typically, a class distinction represents an idealized expression pattern, where the expression level of a gene is uniformly high for samples in one class and uniformly low for samples in the other class.

The correlation between “g” and the class distinction can be measured by a signal-to-noise score:

P(g,c)=[□₁(g)−□₂(g)]/[□₁(g)+□₂(g)]

where □₁(g) and □₂(g) represent the means of the log-transformed expression levels of gene “g” in class 0 and class 1, respectively, and □₁(g) and □₂(g) represent the standard deviation of the log-transformed expression levels of gene “g” in class 0 and class 1, respectively. A higher absolute value of a signal-to-noise score indicates that the gene is more highly expressed in one class than in the other. In one example, the samples used to derive the signal-to-noise scores comprise enriched or purified PBMCs and, therefore, the signal-to-noise score P(g,c) represents the correlation between the class distinction and the expression level of gene “g” in PBMCs.

The correlation between gene “g” and the class distinction can also be measured by other methods, such as by the Pearson correlation coefficient or the Euclidean distance, as appreciated by those skilled in the art.

The significance of the correlation between marker expression profiles and the class distinction is evaluated using a random permutation test. An unusually high density of genes within the neighborhoods of the class distinction, as compared to random patterns, suggests that many genes have expression patterns that are significantly correlated with the class distinction. The correlation between genes and the class distinction can be diagrammatically viewed through a neighborhood analysis plot, in which the y-axis represents the number of genes within various neighborhoods around the class distinction and the x-axis indicates the size of the neighborhood (i.e., P(g,c)). Curves showing different significance levels for the number of genes within corresponding neighborhoods of randomly permuted class distinctions can also be included in the plot.

In many embodiments, the markers employed in the present invention are above the median significance level in the neighborhood analysis plot. This means that the correlation measure P(g,c) for each marker is such that the number of genes within the neighborhood of the class distinction having the size of P(g,c) is greater than the number of genes within the corresponding neighborhoods of random permuted class distinctions at the median significance level. In many other embodiments, the markers employed in the present invention are above the 40%, 30%, 20%, 10%, 5%, 2%, or 1% significance level. As used herein, x % significance level means that x % of random neighborhoods contain as many genes as the real neighborhood around the class distinction.

In another aspect, the correlation between marker expression profiles and health status or clinical outcome can be evaluated by statistical methods. One exemplary statistical method employs Spearman's rank correlation coefficient, which has the formula of:

rₛ=SS_{UV}/(SS_{UU}SS_{VV})^{1/2}

where SS_{UV}=ΣUᵢVᵢ−[(ΣUᵢ)(ΣVᵢ)]/n, SS_{UU}=ΣVᵢ²−[(ΣVᵢ)²]/n, and SS_{VV}=ΣUᵢ²−[(ΣUᵢ)²]/n. Uᵢ is the expression level ranking of a gene of interest, Vᵢ is the ranking of the health status or clinical outcome, and n represents the number of patients. The shortcut formula for Spearman's rank correlation coefficient is rₛ=1−(6×Σdᵢ²)/[n(n²−1)], where dᵢ=Uᵢ−Vᵢ. The Spearman's rank correlation is similar to the Pearson's correlation except that it is based on ranks and is thus more suitable for data that is not normally distributed. See, for example, Snedecor and Cochran, Statistical Methods, Eighth edition, Iowa State University Press, Ames, Iowa, 1989. The correlation coefficient is tested to assess whether it differs significantly from a value of 0 (i.e., no correlation).

The correlation coefficients for each marker identified by the Spearman's rank correlation can be either positive or negative, provided that the correlation is statistically significant. In many embodiments, the p-value for each marker thus identified is no more than 0.05, 0.01, 0.005, 0.001, 0.0005, 0.0001, or less. In many other embodiments, the Spearman correlation coefficients of the markers thus identified have absolute values of at least 0.3, 0.4, 0.5, 0.6, 0.7, 0.8, 0.9, or more.

Another exemplary statistical method is Cox proportional hazard regression model, which has the formula of:

log hᵢ(t)=α(t)+βⱼxᵢⱼ

wherein hᵢ(t) is the hazard function that assesses the instantaneous risk of demise at time t, conditional on survival to that time, α(t) is the baseline hazard function, and xᵢⱼ is a covariate which may represent, for example, the expression level of marker j in a peripheral blood sample or other tissue sample. See Cox (1972) Journal of the Royal Statistical Society, Series B 34:187. Additional covariates, such as interactions between covariates, can also be included in Cox proportional hazard model. As used herein, the terms “demise” or “survival” are not limited to real death or survival. Instead, these terms should be interpreted broadly to cover any type of time-associated events. In many cases, the p-values for the correlation under Cox proportional hazard regression model are no more than 0.05, 0.01, 0.005, 0.001, 0.0005, 0.0001, or less. The p-values for the markers identified under Cox proportional hazard regression model can be determined by the likelihood ratio test, Wald test, the Score test, or the log-rank test. In one embodiment, the hazard ratios for the markers thus identified are at least 1.5, 2, 3, 4, 5, or more. In another embodiment, the hazard ratios for the markers thus identified are no more than 0.67, 0.5., 0.33, 0.25., 0.2, or less.

Other rank tests, scores, measurements, or models can also be employed to identify markers whose expression profiles in peripheral blood samples, or other tissue samples, are correlated with clinical outcome of asthma or an IL-13-mediated condition. These tests, scores, measurements, or models can be either parametric or nonparametric, and the regression may be either linear or non-linear. Many statistical methods and correlation/regression models can be carried out using commercially available programs.

Class predictors can be constructed using the markers of the present invention. These class predictors can be used to assign an asthma or IL-13-mediated condition patient of interest to a health status, outcome, or treatment effectiveness class. In one embodiment, the markers employed in a class predictor are limited to those shown to be significantly correlated with a class distinction by the permutation test, such as those at or above the 1%, 2%, 5%, 10%, 20%, 30%, 40%, or 50% significance level. In another embodiment, the PBMC expression level of each marker in a class predictor is substantially higher or substantially lower in one class of patients than in another class of patients. In still another embodiment, the markers in a class predictor have top absolute values of P(g,c). In yet another embodiment, the p-value under a Student's t-test (e.g., two-tailed distribution, two sample unequal variance) for each marker in a class predictor is no more than 0.05, 0.01, 0.005, 0.001, 0.0005, 0.0001, or less. For each marker, the p-value suggests the statistical significance of the difference observed between the average PBMC, or other tissue, expression profiles of the gene in one class of patients versus another class of patients. Lesser p-values indicate more statistical significance for the differences observed between the different classes of asthma or IL-13-mediated condition patients.

The SAM method can also be used to correlate peripheral blood gene expression profiles with different health status, outcome, or treatment effectiveness classes. The prediction analysis of microarrays (PAM) method can then be used to identify class predictors that can best characterize a predefined health status, outcome or treatment effectiveness class and predict the class membership of new samples. See Tibshirani (2002) Proc. Natl. Acad. Sci. U.S.A., 99: 6567-6572.

In many embodiments, a class predictor of the present invention has high prediction accuracy under leave-one-out cross validation, 10-fold cross validation, or 4-fold cross validation. For instance, a class predictor of the present invention can have at least 50%, 60%, 70%, 80%, 90%, 95%, or 99% accuracy under leave-one-out cross validation, 10-fold cross validation, or 4-fold cross validation. In a typical k-fold cross validation, the data is divided into k subsets of approximately equal size. The model is trained k times, each time leaving out one of the subsets from training and using the omitted subset as the test sample to calculate the prediction error. If k equals the sample size, it becomes the leave-one-out cross validation.

Other class-based correlation metrics or statistical methods can also be used to identify markers whose expression profiles in peripheral blood samples, or other tissue samples, are correlated with health status or clinical outcome of asthma or IL-13-mediated condition patients. Many of these methods can be performed by using commercial or publicly accessible software packages.

Other methods capable of identifying asthma markers include, but are not limited to, RT-PCR, Northern blot, in situ hybridization, and immunoassays such as ELISA, RIA, or Western blot. These genes are differentially expressed in peripheral blood cells (e.g., PBMCs), or other tissues, of one class of patients relative to another class of patients. In many cases, the average marker expression level of each of these genes in one class of patients is statistically different from that in another class of patients. For instance, the p-value under an appropriate statistical significance test (e.g., Student's t-test) for the observed difference can be no more than 0.05, 0.01, 0.005, 0.001, 0.0005, 0.0001, or less. In many other cases, each marker thus identified has at least 2-, 3-, 4-, 5-, 10-, or 20-fold difference in the average PBMC, or other tissue, expression level between one class of patients and another class of patients.

### Asthma and IL-13-Mediated Condition Treatment

Any asthma treatment regime, or regime for treatment of an IL-13-mediated condition, and its effectiveness, can be analyzed according to the present invention. Examples of these treatments include, but are not limited to, drug therapy, gene therapy, radiation therapy, immunotherapy, biological therapy, surgery, or a combination thereof. Other conventional, non-conventional, novel, or experimental therapies, including treatments under clinical trials, can also be evaluated according to the present invention.

A variety of anti-asthma, anti-inflammatory, or anti-allergy agents can be used to treat asthma or an IL-13-mediated condition. An “asthma/allergy medicament” as used herein is a composition of matter which reduces the symptoms, inhibits the asthmatic or allergic reaction, or prevents the development of an allergic or asthmatic reaction. Various types of medicaments for the treatment of asthma and allergy are described in the Guidelines For The Diagnosis and Management of Asthma, Expert Panel Report 2, NIH Publication No. 97/4051, Jul. 19, 1997, the entire contents of which are incorporated herein by reference. The summary of the medicaments as described in the NIH publication is presented below. Examples of useful medicaments according to the present invention that are either on the market or in development are presented in Tables 3 and 4.

In most embodiments the asthma/allergy medicament is useful to some degree for treating both asthma and allergy, particularly IL-13-mediated conditions. Treatments for conditions mediated by IL-13 include, but are not limited to, IL-13 antagonists, soluble IL-13 receptor-Fc fusion proteins, IL-13 antibodies, and nucleic acids, either via antisense, RNA interference (RNAi) or gene therapeutic technologies. Asthma medicaments include, but are not limited, PDE-4 inhibitors, bronchodilator/beta-2 agonists, beta-2 adrenoreceptor ant/agonists, anticholinergics, steroids, K⁺ channel openers, VLA-4 antagonists, neurokin antagonists, thromboxane A2 synthesis inhibitors, xanthines, arachidonic acid antagonists, 5 lipoxygenase inhibitors, thromboxin A2 receptor antagonists, thromboxane A2 antagonists, inhibitor of 5-lipox activation proteins, protease inhibitors, and nucleic acids, either via antisense, RNA interference (RNAi) or gene therapeutic technologies.

Bronchodilator/beta-2 agonists are a class of compounds which cause bronchodilation or smooth muscle relaxation. Bronchodilator/beta-2 agonists include, but are not limited to, salmeterol, salbutamol, albuterol, terbutaline, D2522/formoterol, fenoterol, bitolterol, pirbuerol, methylxanthines and orciprenaline. Long-acting beta-2 agonists and bronchodilators are compounds which are used for long-term prevention of symptoms in addition to the anti-inflammatory therapies. They function by causing bronchodilation, or smooth muscle relaxation, following adenylate cyclase activation and increase in cyclic AMP producing functional antagonism of bronchoconstriction. These compounds also inhibit mast cell mediator release, decrease vascular permeability and increase mucociliary clearance. Long-acting beta-2 agonists include, but are not limited to, salmeterol and albuterol. These compounds are usually used in combination with corticosteroids and generally are not used without any inflammatory therapy. They have been associated with side effects such as tachycardia, skeletal muscle tremor, hypokalemia, and prolongation of QTc interval in overdose.

Methylxanthines, including for instance theophylline, have been used for long-term control and prevention of symptoms. These compounds cause bronchodilation resulting from phosphodiesterase inhibition and likely adenosine antagonism. It is also believed that these compounds may effect eosinophilic infiltration into bronchial mucosa and decrease T-lymphocyte numbers in the epithelium. Dose-related acute toxicities are a particular problem with these types of compounds. As a result, routine serum concentration should be monitored in order to account for the toxicity and narrow therapeutic range arising from individual differences in metabolic clearance. Side effects include tachycardia, nausea and vomiting, tachyarrhythmias, central nervous system stimulation, headache, seizures, hematemesis, hyperglycemia and hypokalemia. Short-acting beta-2 agonists/bronchodilators relax airway smooth muscle, causing the increase in air flow. These types of compounds are a preferred drug for the treatment of acute asthmatic systems. Previously, short-acting beta-2 agonists had been prescribed on a regularly-scheduled basis in order to improve overall asthma symptoms. Later reports, however, suggested that regular use of this class of drugs produced significant diminution in asthma control and pulmonary function (Sears (1990) Lancet, 336:1391-6). Other studies showed that regular use of some types of beta-2 agonists produced no harmful effects over a four-month period but also produced no demonstrable effects (Drazen (1996) N. Eng. J. Med., 335:841-7). As a result of these studies, the daily use of short-acting beta-2 agonists is not generally recommended. Short-acting beta-2 agonists include, but are not limited to, albuterol, bitolterol, pirbuterol, and terbutaline. Some of the adverse effects associated with the mastration of short-acting beta-2 agonists include tachycardia, skeletal muscle tremor, hypokalemia, increased lactic acid, headache, and hyperglycemia.

Other allergy medicaments are commonly used in the treatment of asthma. These include, but are not limited to, anti-histamines, steroids, and prostaglandin inducers. Anti-histamines are compounds which counteract histamine released by mast cells or basophils. Anti-histamines include, but are not limited to, loratidine, cetirizine, buclizine, ceterizine analogues, fexofenadine, terfenadine, desloratadine, norastemizole, epinastine, ebastine, astemizole, levocabastine, azelastine, tranilast, terfenadine, mizolastine, betatastine, CS 560, and HSR 609. Prostaglandins function by regulating smooth muscle relaxation. Prostaglandin inducers include, but are not limited to, S-575 1.

The steroids include, but are not limited to, beclomethasone, fluticasone, tramcinolone, budesonide, corticosteroids and budesonide. To date, the use of steroids in children has been limited by the observation that some steroid treatments have been reportedly associated with growth retardation.

Corticosteroids are used long-term to prevent development of the symptoms, and suppress, control, and reverse inflammation arising from an initiator. Some corticosteroids can be administered by inhalation and others are administered systemically. The corticosteroids that are inhaled have an anti-inflammatory function by blocking late-reaction allergen and reducing airway hyper-responsiveness. These drugs also inhibit cytokine production, adhesion protein activation, and inflammatory cell migration and activation.

Corticosteroids include, but are not limited to, beclomethasome dipropionate, budesonide, flunisolide, fluticaosone, propionate, and triamcinoone acetonide. Although dexamethasone is a corticosteroid having anti-inflammatory action, it is not regularly used for the treatment of asthma/allergy in an inhaled form because it is highly absorbed and it has long-term suppressive side effects at an effective dose. Dexamethasone, however, can be administered at a low dose to reduce the side effects. Some of the side effects associated with corticosteroid include cough, dysphonia, oral thrush (candidiasis), and in higher doses, systemic effects, such as adrenal suppression, osteoporosis, growth suppression, skin thinning and easy bruising. (Barnes (1993) Am. J. Respir. Crit. Care Med., 153:1739-48)

Systemic corticosteroids include, but are not limited to, methylprednisolone, prednisolone and prednisone. Corticosteroids are used generally for moderate to severe exacerbations to prevent the progression, reverse inflammation and speed recovery. These anti-inflammatory compounds include, but are not limited to, methylprednisolone, prednisolone, and prednisone. Corticosteroids are associated with reversible abnormalities in glucose metabolism, increased appetite, fluid retention, weight gain, mood alteration, hypertension, peptic ulcer, and rarely asceptic necrosis of femur. These compounds are useful for short-term (3-10 days) prevention of the inflammatory reaction in inadequately controlled persistent asthma. They also function in a long-term prevention of symptoms in severe persistent asthma to suppress and control and actually reverse inflammation. The side effects associated with systemic corticosteroids are even greater than those associated with inhaled corticosteroids. Side effects include, for instance, reversible abnormalities in glucose metabolism, increased appetite, fluid retention, weight gain, mood alteration, hypertension, peptic ulcer and asceptic necrosis of femur, which are associated with short-term use. Some side effects associated with longer term use include adrenal axis suppression, growth suppression, dermal thinning, hypertension, diabetes, Cushing's syndrome, cataracts, muscle weakness, and in rare instances, impaired immune function. The inhaled corticosteroids are believed to function by blocking late reaction to allergen and reducing airway hyper-responsiveness. They are also believed to reverse beta-2-receptor downregulation and to inhibit microvascular leakage.

The immunomodulators include, but are not limited to, the group consisting of anti-inflammatory agents, leukotriene antagonists, IL-4 muteins, soluble IL-4 receptors, immunosuppressants (such as tolerizing peptide vaccine), IL-4 antagonists, anti-IL-5 antibodies, anti-IL-9 antibodies, CCR3 antagonists, CCR5 antagonists, VLA-4 inhibitors, and, and downregulators of IgE.

Leukotriene modifiers are often used for long-term control and prevention of symptoms in mild persistent asthma. Leukotriene modifiers function as leukotriene receptor antagonists by selectively competing for LTD-4 and LTE-4 receptors. These compounds include, but are not limited to, zafirlukast tablets and zileuton tablets. Zileuton tablets function as 5-lipoxygenase inhibitors. These drugs have been associated with the elevation of liver enzymes and some cases of reversible hepatitis and hyperbilirubinemia. Leukotrienes are biochemical mediators that are released from mast cells, eosinophils, and basophils that cause contraction of airway smooth muscle and increase vascular permeability, mucous secretions and activate inflammatory cells in the airways of patients with asthma.

Other immunomodulators include neuropeptides that have been shown to have immunomodulating properties. Functional studies have shown that substance P, for instance, can influence lymphocyte function by specific receptor mediated mechanisms. Substance P also has been shown to modulate distinct immediate hypersensitivity responses by stimulating the generation of arachidonic acid-derived mediators from mucosal mast cells (McGillies (1987) Fed. Proc., 46:196-9). Substance P is a neuropeptide first identified in 1931 by Von Euler (Von Euler (1931) J. Physiol. (London), 72:74-87). Its amino acid sequence was reported by Chang (Chang (1971) Nature (London) 232:86-87). The immunoregulatory activity of fragments of substance P has been studied by Siemion (Siemion (1990) Molec. Immunol., 27:887-890).

Another class of compounds is the down-regulators of IgE. These compounds include peptides or other molecules with the ability to bind to the IgE receptor and thereby prevent binding of antigen-specific IgE. Another type of downregulator of IgE is a monoclonal antibody directed against the IgE receptor-binding region of the human IgE molecule. Thus, one type of downregulator of IgE is an anti-IgE antibody or antibody fragment. One of skill in the art could prepare functionally active antibody fragments of binding peptides which have the same function. Other types of IgE downregulators are polypeptides capable of blocking the binding of the IgE antibody to the Fc receptors on the cell surfaces and displacing IgE from binding sites upon which IgE is already bound.

One problem associated with downregulators of IgE is that many molecules lack a binding strength to the receptor corresponding to the very strong interaction between the native IgE molecule and its receptor. The molecules having this strength tend to bind irreversibly to the receptor. However, such substances are relatively toxic since they can bind covalently and block other structurally similar molecules in the body. Of interest in this context is that the alpha chain of the IgE receptor belongs to a larger gene family of different IgG Fc receptors. These receptors are absolutely essential for the defense of the body against bacterial infections. Molecules activated for covalent binding are, furthermore, often relatively unstable and therefore they probably have to be administered several times a day and then in relatively high concentrations in order to make it possible to block completely the continuously renewing pool of IgE receptors on mast cells and basophilic leukocytes.

These types of asthma/allergy medicaments are sometimes classified as long-term control medications or quick-relief medications. Long-term control medications include compounds such as corticosteroids (also referred to as glucocorticoids), methylprednisolone, prednisolone, prednisone, cromolyn sodium, nedocromil, long-acting beta-2-agonists, methylxanthines, and leukotriene modifiers. Quick relief medications are useful for providing quick relief of symptoms arising from allergic or asthmatic responses. Quick relief medications include short-acting beta-2 agonists, anticholinergics and systemic corticosteroids.

Chromolyn sodium and medocromil are used as long-term control medications for preventing primarily asthma symptoms arising from exercise or allergic symptoms arising from allergens. These compounds are believed to block early and late reactions to allergens by interfering with chloride channel function. They also stabilize mast cell membranes and inhibit activation and release of mediators from eosinophils and epithelial cells. A four to six week period of administration is generally required to achieve a maximum benefit.

Anticholinergics are generally used for the relief of acute bronchospasm. These compounds are believed to function by competitive inhibition of muscarinic cholinergic receptors. Anticholinergics include, but are not limited to, ipratrapoium bromide. These compounds reverse only cholinerigically-mediated bronchospasm and do not modify any reaction to antigen. Side effects include drying of the mouth and respiratory secretions, increased wheezing in some individuals, blurred vision if sprayed in the eyes.

In addition to standard asthma/allergy medicaments other methods for treating asthma/allergy have been used either alone or in combination with established medicaments. One preferred, but frequently impossible, method of relieving allergies is allergen or initiator avoidance. Another method currently used for treating allergic disease involves the injection of increasing doses of allergen to induce tolerance to the allergen and to prevent further allergic reactions.

Allergen injection therapy (allergen immunotherapy) is known to reduce the severity of allergic rhinitis. This treatment has been theorized to involve the production of a different form of antibody, a protective antibody which is termed a “blocking antibody” (Cooke (1935) Exp. Med., 62:733). Other attempts to treat allergy involve modifying the allergen chemically so that its ability to cause an immune response in the patient is unchanged, while its ability to cause an allergic reaction is substantially altered.

Commonly used allergy and asthma drugs which are currently in development or on the market are shown in Tables 3 and 4 respectively.

### Arrays

In yet another embodiment, the present invention provides arrays (including low density microarrays) that are used for detecting or comparing the expression profiles of an asthma or IL-13-responsive marker of interest. In a preferred embodiment, the present invention provides arrays for detecting or hybridizing to the markers of Table 1a and b. In another embodiment, the present invention provides arrays for detecting or hybridizing to the markers in Table 1b wherein “yes” is indicated in Column C. In some embodiments, nucleic acid arrays are provided. In another embodiment, the array can be an antibody, or other polypeptide, array. The nucleic acid arrays can be commercial oligonucleotide or cDNA arrays. They can also be custom arrays comprising concentrated probes for the markers of the present invention. In many examples, at least 15%, 20%, 25%, 30%, 35%, 40%, 45%, 50%, or more of the total probes on a custom array of the present invention are probes for asthma markers or markers for IL-13 responsiveness. These probes can hybridize under stringent or nucleic acid array hybridization conditions to the RNA transcripts, or the complements thereof, of the corresponding markers.

As used herein, “stringent conditions” are at least as stringent as, for example, conditions G-L shown in Table 5. “Highly stringent conditions” are at least as stringent as conditions A-F shown in Table 5.

In one example, a nucleic acid array of the present invention includes at least 2, 5, 10, or more different probes. Each of these probes is capable of hybridizing under stringent or nucleic acid array hybridization conditions to a different respective marker of the present invention. Multiple probes for the same marker can be used on the same nucleic acid array. The probe density on the array can be in any range.

The probes for a marker of the present invention can be a nucleic acid probe, such as, DNA, RNA, PNA, or a modified form thereof. The nucleotide residues in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

The probes for the markers can be stably attached to discrete regions, or addresses, on a nucleic acid array. By “stably attached,” or “affixed thereto,” or “disposed thereon,” it is intended that a probe maintains its position relative to the attached discrete region, or address, during hybridization and signal detection. The position of each discrete region, or address, on the nucleic acid array can be either known or determinable. All of the methods known in the art can be used to make the nucleic acid arrays or antibody/protein arrays of the present invention.

In another aspect, the present invention provides an array for detecting a marker differentially expressed in asthma or responsive to exposure to IL-13. In another embodiment, the array is for use in a method for predicting a clinical outcome for an asthma patient. The array of the invention includes a substrate having a plurality of addresses, each of which has a distinct probe disposed thereon or affixed thereto. In one embodiment, at least 10% of the plurality of addresses have affixed thereto or disposed thereon probes that can specifically detect or hybridize to markers for asthma or IL-13 responsiveness. In some embodiments, at least 15% of the plurality of addresses have disposed thereon or affixed thereto probes that can specifically detect or hybridize to markers of asthma or IL-13 responsiveness in PBMCs or other tissues. In some embodiments, at least 20% of the plurality of addresses have disposed thereon or affixed thereto probes that can specifically detect or hybridize to markers of asthma or IL-13 responsiveness in PBMCs or other tissues. In some embodiments, at least 25% of the plurality of addresses have disposed thereon or affixed thereto probes that can specifically detect or hybridize to markers of asthma or IL-13 responsiveness in PBMCs or other tissues. In some embodiments, at least 30% of the plurality of addresses have disposed thereon or affixed thereto probes that can specifically detect or hybridize to markers of IL-13 responsiveness or asthma in PBMCs or other tissues. In some embodiments, at least 40% of the plurality of addresses have disposed thereon or affixed thereto probes that can specifically detect or hybridize to markers of asthma or IL-13 responsiveness in PBMCs or other tissues. In some embodiments, at least 50% of the plurality of addresses have disposed thereon or affixed thereto probes that can specifically detect or hybridize to markers of IL-13 responsiveness or asthma in PBMCs or other tissues. In some embodiments, at least 60% of the plurality of addresses have disposed thereon or affixed thereto probes that can specifically detect or hybridize to markers of asthma or IL-13 responsiveness in PBMCs or other tissues. In some embodiments, at least 70% of the plurality of addresses have disposed thereon or affixed thereto probes that can specifically detect or hybridize to markers of asthma or IL-13 responsiveness in PBMCs or other tissues. In some embodiments, at least 80% of the plurality of addresses have disposed thereon or affixed thereto probes that can specifically detect or hybridize to markers of asthma or IL-13 responsiveness in PBMCs or other tissues. In some embodiments, at least 90% of the plurality of addresses have disposed thereon or affixed thereto probes that can specifically detect or hybridize to markers of asthma or IL-13 responsiveness in PBMCs or other tissues. In some embodiments, the markers are selected from Table 1a and b. In other embodiments, the markers are selected from the markers in Table 1b wherein “yes” is indicated in Column C. In some embodiments, at least 10%, 15%, 20%, 30%, 40%, 50%, 60%, 70%, 80%, or at least 90% of the plurality of addresses have disposed thereon or affixed thereto markers selected from Table 1a and b. In some embodiments, at least 10%, 15%, 20%, 30%, 40%, 50%, 60%, 70%, 80%, or at least 90% of the plurality of addresses have disposed thereon or affixed thereto markers selected from the markers in Table 1b wherein “yes” is indicated in Column C. In some embodiments, the array of the present invention has affixed to or disposed thereon at least 5, preferably at least 10, at least 15, at least 20, at least 25, at least 30, at least 35, at least 40, at least 45, at least 50, at least 60, at least 70, at least 80, at least 90, at least 100, or at least 150 markers selected from Table 1a and b. In some embodiments, the array of the present invention has affixed to or disposed thereon at least 5, preferably at least 10, at least 15, at least 20, at least 25, at least 30, at least 35, at least 40, at least 45, at least 50, at least 60, or at least 70 markers selected from Table 1b wherein “yes” is indicated in Column C. The probe suitable for the present invention may be a nucleic acid probe. Alternatively, the probe suitable for the present invention may be an antibody probe.

In a further aspect, the present invention provides an array for use in a method for diagnosis of asthma or an IL-13-mediated condition including a substrate having a plurality of addresses, each of which have a distinct probe disposed thereon or affixed thereto. In one embodiment, at least 10% of the plurality of addresses have affixed thereto or disposed thereon probes that can specifically detect or hybridize to markers for asthma or IL-13 responsiveness. In some embodiments, at least 15% of the plurality of addresses have disposed thereon or affixed thereto probes that can specifically detect or hybridize to markers of asthma or IL-13 responsiveness in PBMCs or other tissues. In some embodiments, at least 20% of the plurality of addresses have disposed thereon or affixed thereto probes that can specifically detect or hybridize to markers of asthma or IL-13 responsiveness in PBMCs or other tissues. In some embodiments, at least 25% of the plurality of addresses have disposed thereon or affixed thereto probes that can specifically detect or hybridize to markers of asthma or IL-13 responsiveness in PBMCs or other tissues. In some embodiments, at least 30% of the plurality of addresses have disposed thereon or affixed thereto probes that can specifically detect or hybridize to markers of IL-13 responsiveness or asthma in PBMCs or other tissues. In some embodiments, at least 40% of the plurality of addresses have disposed thereon or affixed thereto probes that can specifically detect or hybridize to markers of asthma or IL-13 responsiveness in PBMCs or other tissues. In some embodiments, at least 50% of the plurality of addresses have disposed thereon or affixed thereto probes that can specifically detect or hybridize to markers of IL-13 responsiveness or asthma in PBMCs or other tissues. In some embodiments, at least 60% of the plurality of addresses have disposed thereon or affixed thereto probes that can specifically detect or hybridize to markers of asthma or IL-13 responsiveness in PBMCs or other tissues. In some embodiments, at least 70% of the plurality of addresses have disposed thereon or affixed thereto probes that can specifically detect or hybridize to markers of asthma or IL-13 responsiveness in PBMCs or other tissues. In some embodiments, at least 80% of the plurality of addresses have disposed thereon or affixed thereto probes that can specifically detect or hybridize to markers of asthma or IL-13 responsiveness in PBMCs or other tissues. In some embodiments, at least 90% of the plurality of addresses have disposed thereon or affixed thereto probes that can specifically detect or hybridize to markers of asthma or IL-13 responsiveness in PBMCs or other tissues. In some embodiments, the markers are selected from Table 1a and b. In other embodiments, the markers are selected from the markers in Table 1b wherein “yes” is indicated in Column C. In some embodiments, at least 10%, 15%, 20%, 30%, 40%, 50%, 60%, 70%, 80%, or at least 90% of the plurality of addresses have disposed thereon or affixed thereto markers selected from Table 1a and b. In some embodiments, at least 10%, 15%, 20%, 30%, 40%, 50%, 60%, 70%, 80%, or at least 90% of the plurality of addresses have disposed thereon or affixed thereto markers selected from the markers in Table 1b wherein “yes” is indicated in Column C. In some embodiments, the array of the present invention has affixed to or disposed thereon at least 5, preferably at least 10, at least 15, at least 20, at least 25, at least 30, at least 35, at least 40, at least 45, at least 50, at least 60, at least 70, at least 80, at least 90, at least 100, or at least 150 markers selected from Table 1a and b. In some embodiments, the array of the present invention has affixed to or disposed thereon at least 5, preferably at least 10, at least 15, at least 20, at least 25, at least 30, at least 35, at least 40, at least 45, at least 50, at least 60, or at least 70 markers selected from Table 1b wherein “yes” is indicated in Column C. The probe suitable for the present invention may be a nucleic acid probe. Alternatively, the probe suitable for the present invention may be an antibody probe.

In a further aspect, the present invention provides a low density array for use in a method of diagnosis, prognosis, or assessment of asthma or an IL-13-mediated condition or determination of IL-13 responsiveness, including a substrate having a plurality of addresses, each of which has a distinct probe disposed thereon or affixed thereto. The low density array provides the benefit of lower cost, given the lower number of probes that are required to be disposed upon or affixed to the array. Furthermore, the low density array also provides a higher sensitivity given the greater representation of a select number of probes of interest as a percentage of all probes at all addresses on the array. In one embodiment, the present invention provides a low density array for use in assessing a patient's asthma or IL-13-mediated condition or IL-13 responsiveness. In another embodiment, the present invention provides a low density array for use in evaluating or identifying agents capable of modulating the level of expression of markers that are differentially expressed in asthma or IL-13-mediated condition or are responsive to IL-13. In one embodiment, the low density array is capable of hybridizing to at least 10 markers selected from Table 1a and b. In another embodiment, the low density array is capable of hybridizing to at least 20 markers selected from Table 1a and b. In one embodiment, at least 10% of the plurality of addresses have affixed thereto or disposed thereon probes that can specifically detect or hybridize to markers for asthma or IL-13 responsiveness. In some embodiments, at least 15% of the plurality of addresses have disposed thereon or affixed thereto probes that can specifically detect or hybridize to markers of asthma or IL-13 responsiveness in PBMCs or other tissues. In some embodiments, at least 20% of the plurality of addresses have disposed thereon or affixed thereto probes that can specifically detect or hybridize to markers of asthma or IL-13 responsiveness in PBMCs or other tissues. In some embodiments, at least 25% of the plurality of addresses have disposed thereon or affixed thereto probes that can specifically detect or hybridize to markers of asthma or IL-13 responsiveness in PBMCs or other tissues. In some embodiments, at least 30% of the plurality of addresses have disposed thereon or affixed thereto probes that can specifically detect or hybridize to markers of IL-13 responsiveness or asthma in PBMCs or other tissues. In some embodiments, at least 40% of the plurality of addresses have disposed thereon or affixed thereto probes that can specifically detect or hybridize to markers of asthma or IL-13 responsiveness in PBMCs or other tissues. In some embodiments, at least 50% of the plurality of addresses have disposed thereon or affixed thereto probes that can specifically detect or hybridize to markers of IL-13 responsiveness or asthma in PBMCs or other tissues. In some embodiments, at least 60% of the plurality of addresses have disposed thereon or affixed thereto probes that can specifically detect or hybridize to markers of asthma or IL-13 responsiveness in PBMCs or other tissues. In some embodiments, at least 70% of the plurality of addresses have disposed thereon or affixed thereto probes that can specifically detect or hybridize to markers of asthma or IL-13 responsiveness in PBMCs or other tissues. In some embodiments, at least 80% of the plurality of addresses have disposed thereon or affixed thereto probes that can specifically detect or hybridize to markers of asthma or IL-13 responsiveness in PBMCs or other tissues. In some embodiments, at least 90% of the plurality of addresses have disposed thereon or affixed thereto probes that can specifically detect or hybridize to markers of asthma or IL-13 responsiveness in PBMCs or other tissues. In some embodiments, the markers are selected from Table 1a and b. In other embodiments, the markers are selected from the markers in Table 1b wherein “yes” is indicated in Column C. In some embodiments, at least 10%, 15%, 20%, 30%, 40%, 50%, 60%, 70%, 80%, or at least 90% of the plurality of addresses have disposed thereon or affixed thereto markers selected from Table 1a and b. In some embodiments, at least 10%, 15%, 20%, 30%, 40%, 50%, 60%, 70%, 80%, or at least 90% of the plurality of addresses have disposed thereon or affixed thereto markers selected from the markers in Table 1b wherein “yes” is indicated in Column C. In some embodiments, the array of the present invention has affixed to or disposed thereon at least 5, preferably at least 10, at least 15, at least 20, at least 25, at least 30, at least 35, at least 40, at least 45, at least 50, at least 60, at least 70, at least 80, at least 90, at least 100, or at least 150 markers selected from Table 1a and b. In some embodiments, the array of the present invention has affixed to or disposed thereon at least 5, preferably at least 10, at least 15, at least 20, at least 25, at least 30, at least 35, at least 40, at least 45, at least 50, at least 60, or at least 70 markers selected from Table 1b wherein “yes” is indicated in Column C. The probe suitable for the present invention may be a nucleic acid probe. Alternatively, the probe suitable for the present invention may be an antibody probe.

### Screening Methods

The invention also provides methods (also referred to herein as “screening assays”) for identifying agents capable of modulating marker expression (“modulators”), i.e., candidate or test compounds or agents comprising therapeutic moieties (e.g., peptides, peptidomimetics, peptoids, polynucleotides, small molecules or other drugs) which (a) bind to a marker gene product or (b) have a modulatory (e.g., upregulation or downregulation; stimulatory or inhibitory; potentiation/induction or suppression) effect on the activity of a marker gene product or, more specifically, (c) have a modulatory effect on the interactions of the marker gene product with one or more of its natural substrates, or (d) have a modulatory effect on the expression of the marker. Such assays typically comprise a reaction between the marker gene product and one or more assay components. The other components may be either the test compound itself, or a combination of test compound and a binding partner of the marker gene product.

The test compounds of the present invention are generally either small molecules or biomolecules. Small molecules include, but are not limited to, inorganic molecules and small non-biological organic molecules. Biomolecules include, but are not limited to, naturally-occurring and synthetic compounds that have a bioactivity in mammals, such as polypeptides, polysaccharides, and polynucleotides. In one embodiment, the test compound is a small molecule. In another embodiment, the test compound is a biomolecule. One skilled in the art will appreciate that the nature of the test compound may vary depending on the nature of the protein encoded by the marker of the present invention.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckerman (1994) J. Med. Chem., 37:2678-85; spatially addressable parallel solid phase or solution phase libraries; synthetic library methods requiring deconvolution; the “one-bead, one-compound” library method; and synthetic library methods using affinity chromatography selection. The biological library and peptoid library approaches are applicable to peptide, non-peptide oligomers or small molecule libraries of compound (Lam (1997) Anticancer Drug Des., 12:145).

The invention provides methods of screening test compounds for inhibitors of the marker gene products of the present invention. The method of screening comprises obtaining samples from subjects diagnosed with or suspected of having asthma or an IL-13-mediated condition, contacting each separate aliquot of the samples with one or more of a plurality of test compounds, and comparing expression of one or more marker gene products in each of the aliquots to determine whether any of the test compounds provides a substantially decreased level of expression or activity of a marker gene product relative to samples with other test compounds or relative to an untreated sample or control sample. In addition, methods of screening may be devised by combining a test compound with a protein and thereby determining the effect of the test compound on the protein.

In addition, the invention is further directed to a method of screening for test compounds capable of modulating with the binding of a marker gene product and a binding partner, by combining the test compound, the marker gene product, and binding partner together and determining whether binding of the binding partner and the marker gene product occurs. The test compound may be either a small molecule or a biomolecule.

Modulators of marker gene product expression, activity or binding ability are useful as therapeutic compositions of the invention. Such modulators (e.g., antagonists or agonists) may be formulated as compositions or pharmaceutical compositions, as described herein below. Such modulators may also be used in the methods of the invention, for example, to diagnose, treat, or prognose asthma or an IL-13-mediated condition.

The invention provides methods of conducting high-throughput screening for test compounds capable of inhibiting activity or expression of a marker gene product of the present invention. In one embodiment, the method of high-throughput screening involves combining test compounds and the marker gene product and detecting the effect of the test compound on the marker gene product.

A variety of high-throughput functional assays well-known in the art may be used in combination to screen and/or study the reactivity of different types of activating test compounds. Since the coupling system is often difficult to predict, a number of assays may need to be configured to detect a wide range of coupling mechanisms. A variety of fluorescence-based techniques is well-known in the art and is capable of high-throughput and ultra high throughput screening for activity, including but not limited to BRET™ or FRET™ (both by Packard Instrument Co., Meriden, Conn.). The ability to screen a large volume and a variety of test compounds with great sensitivity permits for analysis of the therapeutic targets of the invention to further provide potential inhibitors of asthma or an IL-13-mediated condition. The BIACORE™ system may also be manipulated to detect binding of test compounds with individual components of the therapeutic target, to detect binding to either the encoded protein or to the ligand.

Therefore, the invention provides for high-throughput screening of test compounds for the ability to inhibit activity of a protein encoded by the marker gene products listed in Table 1a and b, by combining the test compounds and the protein in high-throughput assays such as BIACORE™, or in fluorescence-based assays such as BRET™. In addition, high-throughput assays may be utilized to identify specific factors which bind to the encoded proteins, or alternatively, to identify test compounds which prevent binding of the receptor to the binding partner. In the case of orphan receptors, the binding partner may be the natural ligand for the receptor. Moreover, the high-throughput screening assays may be modified to determine whether test compounds can bind to either the encoded protein or to the binding partner (e.g., substrate or ligand) which binds to the protein.

In one embodiment, the high-throughput screening assay detects the ability of a plurality of test compounds to bind to a marker gene product selected from the group consisting of the markers listed in Table 1a and b. In some embodiments, the high-throughput screening assay detects the ability of a plurality of test compounds to bind to a marker gene product selected from the group consisting of markers in Table 1b wherein “yes” is indicated in Column C. In another specific embodiment, the high-throughput screening assay detects the ability of a plurality of a test compound to inhibit a binding partner (such as a ligand) to bind to a marker gene product selected from the group consisting of the markers listed in Table 1a and b. In another specific embodiment, the high-throughput screening assay detects the ability of a plurality of a test compound to inhibit a binding partner (such as a ligand) to bind to a marker gene product selected from the group consisting of markers in Table 1b wherein “yes” is indicated in Column C. In yet another specific embodiment, the high-throughput screening assay detects the ability of a plurality of a test compounds to modulate signaling through a marker gene product selected from the group consisting of the markers listed in Table 1a and b. In another specific embodiment, the high-throughput screening assay detects the ability of a plurality of a test compounds to modulate signaling through a marker gene product selected from the group consisting of the markers in Table 1b wherein “yes” is indicated in Column C.

In one embodiment, one or more candidate agents are administered in vitro directly to cells derived from healthy volunteers and/or asthma or IL-13-mediated condition patients (either before or after treatment). In another particular embodiment, healthy volunteers and/or asthma or IL-13-mediated condition patients are administered one or more candidate agent directly in any manner currently known to, and commonly used by the skilled artisan including generally, but not limited to, enteral or parenteral administration.

### Electronic Systems

The present invention also features electronic systems useful for the prognosis, diagnosis, or selection of treatment of asthma or an IL-13-mediated condition. These systems include an input or communication device for receiving the expression profile of a patient of interest or the reference expression profile(s). The reference expression profile(s) can be stored in a database or other media. The comparison between expression profiles can be conducted electronically, such as through a processor or computer. The processor or computer can execute one or more programs which compare the expression profile of the patient of interest to the reference expression profile(s), the programs can be stored in a memory or other storage media or downloaded from another source, such as an internet server. In one example, the electronic system is coupled to a nucleic acid array and can receive or process expression data generated by the nucleic acid array. In another example, the electronic system is coupled to a protein array and can receive or process expression data generated by the protein array.

### Compositions and Pharmaceutical Compositions

The invention is further directed to compositions and pharmaceutical compositions comprising an anti-asthma compound, anti-IL-13 compound, or bioactive agent. Alternatively, in a preferred embodiment of the present invention, the compositions and pharmaceutical compositions comprise a marker, a marker gene product, or a marker gene product modulator (i.e., agonist or antagonist), which may further include a marker gene product derivative, and can be formulated as described herein, wherein the marker is selected from Table 1a and b. Alternatively, in a preferred embodiment of the present invention, the compositions and pharmaceutical compositions comprise a marker, a marker gene product, or a marker gene product modulator (i.e., agonist or antagonist), which may further include a marker gene product derivative, and can be formulated as described herein, wherein the marker is selected from those markers in Table 1b wherein “yes” is indicated in Column C. Alternatively, these compositions may include an antibody which specifically binds to a marker gene product of the invention, or its variant, and/or an antisense polynucleotide molecule which is complementary to a marker polynucleotide of the invention and can be formulated as described herein. The compositions of the present invention may also include marker polynucleotides or variants of marker polynucleotides. The compositions of the present invention may also include marker gene product polypeptides or variants of marker gene product polypeptides.

One or more of the markers, variants of markers, marker gene products of the invention, fragments of marker gene products, variants of marker gene products, variants of fragments of marker gene products, marker gene product modulators, or anti-marker gene product antibodies of the invention can be incorporated into pharmaceutical compositions suitable for administration.

Methods for purification and isolation of polynucleotides and polypeptides, particularly the marker polynucleotides, marker gene product polypeptides, and variants thereof are well known in the art. Synthetic methods, both in vivo and in vitro, solid- and liquid-phase, for production of isolated marker polynucleotides, marker gene product polypeptides, and variants thereof are also well known in the art.

Suitable antibodies for the compositions of the present invention include, but are not limited to, polyclonal antibodies, monoclonal antibodies, chimeric antibodies, humanized antibodies, single chain antibodies, Fab fragments, or fragments produced by a Fab expression library. Neutralizing antibodies (i.e., those which inhibit dimer formation) can also be used in the compositions of the present invention. Methods for preparing these antibodies are well known in the art. In one embodiment, the antibodies of the present invention can bind specifically to the corresponding marker gene products or other desired antigens with binding affinities of at least 10⁴ M⁻¹, 10⁵ M⁻¹, 10⁶ M⁻¹, 10⁷ M⁻¹, or more. Methods of assessing binding affinities and specificities are well known in the art.

The present invention provides, in one embodiment, a composition comprising an isolated marker polynucleotide wherein the marker is selected from the markers of Table 1a and b. The present invention also provides a composition comprising an isolated marker polynucleotide wherein the marker is selected from the markers of Table 1b wherein “yes” is indicated in Column C. In another embodiment of the present invention the marker is one of the 5 novel or unknown genes. In another embodiment of the present invention, a composition is provided comprising an isolated marker gene product polypeptide wherein the marker is selected from the markers of Table 1a and b. In another embodiment of the present invention, a composition is provided comprising an isolated marker gene product polypeptide wherein the marker is selected from the markers Table 1b wherein “yes” is indicated in Column C. In another embodiment of the present invention the marker is one of the 5 novel or unknown genes. The present invention further provides a composition comprising an antibody that specifically binds to a marker gene product polypeptide wherein the marker is selected from one of the markers of Table 1a and b. The present invention further provides a composition comprising an antibody that specifically binds to a marker gene product polypeptide wherein the marker is selected from one of the markers of Table 1b wherein “yes” is indicated in Column C. In another aspect of the present invention, a composition is provided that comprises an antibody that specifically binds to a marker gene product polypeptide wherein the marker is one of the 5 novel or unknown genes.

Suitable pharmaceutically acceptable carriers include solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The invention includes methods for preparing pharmaceutical compositions for modulating the expression or activity of a polypeptide or polynucleotide corresponding to a marker gene product of the invention. Such methods comprise formulating a pharmaceutically acceptable carrier with an agent which modulates expression or activity of a polypeptide or polynucleotide corresponding to a marker gene product of the invention. Such compositions can further include additional active agents. Thus, the invention further includes methods for preparing a pharmaceutical composition by formulating a pharmaceutically acceptable carrier with an agent which modulates expression or activity of a polypeptide or polynucleotide corresponding to a marker gene product of the invention and one or more additional bioactive agents.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The pH of the solutions can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols such as manitol, sorbitol, sodium chloride can be included in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a marker gene product or an anti-marker gene product antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, examples of methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers.

It is especially advantageous to formulate oral or parenteral compositions in dosage unit form for ease of administration and uniformity of dosage. Dosage unit form as used herein includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In many embodiments, compounds which exhibit large therapeutic indices are selected. While compounds that exhibit toxic side effects may be used, care should be taken to design a delivery system that targets such compounds to the site of affected tissue in order to minimize potential damage to healthy cells and, thereby, reduce side effects.

The data obtained from the cell culture assays and animal studies can be used in formulating a range of dosage for use in humans. The dosage of such compounds can lie within a range of circulating concentrations that includes the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The marker polynucleotides of the invention, and their variants, can be inserted into gene delivery vectors and used as gene therapy vectors. Furthermore, inhibitors or other modulators of the marker gene products of the invention can be inserted into gene delivery vectors and used as gene therapy vectors. Gene therapy vectors can be delivered to a subject by, for example, intravenous administration, intraportal administration, intrabiliary administration, intra-arterial administration, direct injection into the liver parenchyma, by intramusclular injection, by inhalation, by perfusion, or by stereotactic injection. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### Kits for Prognosis, Diagnosis, or Selection of Treatment of Asthma or an IL-13-Mediated Condition

In addition, the present invention features kits useful for the diagnosis or selection of treatment of asthma or an IL-13-mediated condition. Each kit includes or consists essentially of at least one probe for an asthma or IL-13 responsive marker (e.g., a marker selected from Table 1a and b). Reagents or buffers that facilitate the use of the kit can also be included. Any type of probe can be used in the present invention, such as hybridization probes, amplification primers, antibodies, or any and all other probes commonly used and known to the skilled artisan.

In one embodiment, a kit of the present invention includes or consists essentially of at least 1, 2, 3, 4, 5, 6, 7, 8, 9, 10, or more polynucleotide probes or primers. Each probe/primer can hybridize under stringent conditions or nucleic acid array hybridization conditions to a different respective asthma or IL-13 responsive marker. As used herein, a polynucleotide can hybridize to a gene if the polynucleotide can hybridize to an RNA transcript, or complement thereof, of the gene. In another embodiment, a kit of the present invention includes one or more antibodies, each of which is capable of binding to a polypeptide encoded by a different respective asthma or IL-13 responsive marker.

In one example, a kit of the present invention includes or consists essentially of probes (e.g., hybridization or PCR amplification probes or antibodies) for at least 1, 2, 3, 4, 5, 10, 14, 20, 25, 30, 35, 40, 45, 50, 55, 60, 65, 70, 75, or more genes selected from Table 1a and b. In another embodiment, the kit can contain nucleic acid probes and antibodies to 1, 2, 3, 4, 5, 10, 14, 20, 25, 30, 35, 40, 45, 50, 55, 60, 65, 70, 75, or more genes selected from Table 1a and b.

In another example, a kit of the present invention includes or consists essentially of probes (e.g., hybridization or PCR amplification probes or antibodies) for at least 1, 2, 3, 4, 5, 10, 14, 20, 25, 30, 35, 40, 45, 50, 55, 60, or more genes selected from the markers of Table 1b wherein “yes” is indicated in Column C. In another embodiment, the kit can contain nucleic acid probes and antibodies to 1, 2, 3, 4, 5, 10, 14, 20, 25, 30, 35, 40, 45, 50, 55, 60, or more genes selected from the markers of Table 1b wherein “yes” is indicated in Column C.

The probes employed in the present invention can be either labeled or unlabeled. Labeled probes can be detectable by spectroscopic, photochemical, biochemical, bioelectronic, immunochemical, electrical, optical, chemical, or other suitable means. Exemplary labeling moieties for a probe include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

The kits of the present invention can also have containers containing buffer(s) or reporter means. In addition, the kits can include reagents for conducting positive or negative controls. In one embodiment, the probes employed in the present invention are stably attached to one or more substrate supports. Nucleic acid hybridization or immunoassays can be directly carried out on the substrate support(s). Suitable substrate supports for this purpose include, but are not limited to, glasses, silica, ceramics, nylons, quartz wafers, gels, metals, papers, beads, tubes, fibers, films, membranes, column matrices, or microtiter plate wells. The kits of the present invention may also contain one or more controls, each representing a reference expression level of a marker detectable by one or more probes contained in the kits.

The present invention also allows for personalized treatment of asthma or an IL-13-mediated condition. Numerous treatment options or regimes can be analyzed according to the present invention to identify markers for each treatment regime. The peripheral blood expression profiles of these markers in a patient of interest are indicative of the clinical outcome of the patient and, therefore, can be used for the selection of treatments that have favorable prognoses of the majority of all other available treatments for the patient of interest. The treatment regime with the best prognosis can also be identified.

Treatment selection can be conducted manually or electronically. Reference expression profiles or gene classifiers can be stored in a database. Programs capable of performing algorithms such as the k-nearest-neighbors or weighted voting algorithms can be used to compare the peripheral blood expression profile of a patient of interest to the database to determine which treatment should be used for the patient.

It should be understood that the above-described embodiments and the following examples are given by way of illustration, not limitation. Various changes and modifications within the scope of the present invention will become apparent to those skilled in the art from the present description.

### EXAMPLES

### Example 1

### Asthma and IL-13 Responsive Markers

Analyses were performed to select sequences from 150 unique genes as the top candidate markers to assess the effects of IMA638, an IL-13 antagonist, by Taqman Low Density Array (TLDA). Using a dataset consisting of HG-U133A GeneChip® (Affymetrix) results from 1147 individual visits from 337 non-smoking asthma subjects and 1183 visits from 348 non-smoking healthy subjects, ANCOVA analyses identified genes that, by gene expression level, were most significantly associated with asthma and, on an individual visit basis, showed the highest incidence of a detectable fold change when compared to the average level in healthy subjects.

The list of genes thus identified were compared to lists from three independent in vitro studies, two that identified gene expression changes resulting from exposure of human monocytes to IL-13, and a third that identified the effects of IL-13 antagonism on the 6 day PBMC response to allergen stimulation. Also taken into consideration were the results of two in vivo animal studies—one that identified genes affected by IL-13 instillation in the mouse lung, and the other that identified changes in gene expression levels in PBMCs associated with segmental ascaris lung challenge of non-human primates.

In assigning slots on the TLDA, highest priority was given to genes significantly (i.e., having a false discovery rate, or FDR, of less than 1.0e-5) and consistently (in more than 59% of samples) associated with asthma by gene expression level in PBMC and had an average GeneChip® signal greater than 30, and were significantly (FDR<0.05) affected in vitro by IL-13 or its antagonist. A total of 71 genes met all these requirements and are indicated as having met these requirements with a “yes” in Column C of Table 1b.

The vast majority of the remaining TLDA slots were assigned to genes showing a very highly significant (FDA<1.0e-5) association with asthma by expression levels in PBMC and met at least one of the following criteria: a) average fold change of >1.4 in the comparison of asthma and healthy subjects; b) average fold change >1.25, with intra-subject variability <35% and more than 59% of samples showing an expression level difference with the average of healthy volunteers; and/or c) intra-subject variability <20% and more than 59% of samples showing a detectable expression level difference with the average of healthy volunteers. The remaining slots were assigned to genes that were associated with IL-13 through either the in vitro or animal model studies, even if the incidence of samples that differed from the healthy subject average was less than 59% and the association with asthma did not meet the FDR<1.0e-5 level of significance. Table 1a and b provides a complete list of the genes selected as having satisfied the aforementioned criteria and includes the identities and descriptions of the genes as well as pertinent statistical information. The sequences of the probes identified in Table 1a and b are provided in Table 6.

### Example 2

### Clinical Trial and Data Collection

### Sources of Human Blood Samples

Gene expression levels in PBMC of asthma subjects are determined from samples of subjects enrolled in the Wyeth Asthma Observational Study, as are the determinations of the effects of IL-13 antagonism on the in vitro response of asthma subjects to allergen stimulation. Gene expression levels in healthy volunteer PBMC are determined using samples from the Wyeth Healthy Volunteer Observational Study. The effects of in vitro IL-13 stimulation on monocytes of healthy volunteers, and the effects of IL-13 on the in vitro response of healthy subjects to allergen stimulation are determined using samples from Wyeth employee healthy volunteers. Subjects with asthma and healthy volunteer subjects are recruited. Each site's institutional review board or ethics committee approves the study, and no study-specific procedures are performed before obtaining informed consent from each subject. All asthma subjects are on standard of care treatment of inhaled steroids, and samples are also collected from some patients on systemic steroids. Asthma subjects are categorized as mild persistent, moderate persistent or severe persistent according to the 1997 NIH Guidelines for the Diagnosis and Management of Asthma. Atopic status in asthma subjects is assessed by clinical investigators based on positive skin test, family history, or clinical assessment. Healthy volunteers have no known history of asthma or seasonal allergies.

### Sample Collection

Whole blood samples (8 ml×6 tubes) are collected into cell purification tubes (Becton Dickinson, Franklin Lakes, N.J.) according to the manufacturer's recommendations. Blood samples are collected from asthma and healthy subjects and are shipped overnight at room temperature in a temperature controlled box from the clinical site to a site (either Wyeth or a contract lab) that purifies PBMC and RNA.

### RNA Purification and Microarray Hybridization

RNA is purified using QIA shredders and Rneasy mini kits (Qiagen, Valencia, Calif.). PBMC pellets frozen in RLT lysis buffer containing 1% β-mercaptoethanol are thawed and processed for total RNA isolation using the QIA shredder and Rneasy mini kit. A phenol:chloroform extraction is then performed, and the RNA is repurified using the Rneasy mini kit reagents. Eluted RNA is quantified using a Spectramax96 well plate UV reader (Molecular Devices, Sunnyvale, Calif., USA) monitoring A260/280 OD values. The quality of each RNA sample is assessed by capillary electrophoresis alongside an RNA molecular weight ladder on the Agilent 2100 bioanalyzer (Agilent Technologies, Palo Alto, Calif., USA). RNA samples are assigned quality values of intact (distinct 18S and 28S bands); partially degraded (discernible 18S and 28S bands with presence of low molecular weight bands) or completely degraded (no discernible 18S and 28S bands).

Labeled targets for oligonucleotide arrays are prepared using a modification of the procedure described by Lockhart (Lockhart (1996) Nat. Biotechnol., 14:1675-80). Labeled targets are hybridized to the HG-U133A Affymetrix GeneChip Array as described in the Affymetrix technical manual. Eleven biotinylated control transcripts ranging in abundance from 3 parts per million (ppm) to 100 ppm are spiked into each sample to function as a standard curve (Hill (2001) Genome Biol., 2:RESEARCH0055). GeneChip MAS 5.0 software is used to evaluate the hybridization intensity, compute the signal value for each probe set and make an absent/present call.

### Data Normalization and Filtering

GeneChips are required to pass the pre-set quality control criteria determined by the 5′:3′ ratio of the GAPDH and bActin genes. Samples are excluded from the study if they fail to meet the RNA quality metric. Sequences are excluded from the study of uncultured PBMC if the number of present calls is less than 10% and/or if the proportion of samples with signal greater than 50 is less than 10%. For all the in vitro studies, the signal value for each probe set is converted into a frequency value representative of the number of transcripts present in 10⁶ transcripts by reference to the standard curve (Hill (2001) Genome Biol., 2:RESEARCH0055). Sequences are excluded from the in vitro study if they are not found present in at least five samples and/or do not have a frequency of greater than 10 parts per million (by standard curve) in at least one sample.

### Statistical Analysis

For the PBMC study on samples that are not subjected to culture, the clinical and gene expression databases are merged using SAS, and SAS is used for all analyses. Analyses are conducted to identify factors that might have confounding effects on associations between gene expression levels and response group. Differential blood cell counts, age, sex, race, country, processing laboratory, and sample quality are identified as significant covariates. For each gene, ANCOVA is used to test for associations of expression level with these co-variates. ANCOVA is performed using the Log2 transformed Affymetrix MAS5 signal to identify significant differences in gene expression levels between the asthma and healthy volunteer groups. The fold change differences are calculated by back-transforming the difference in the log 2 least square means. For the in vitro study on the effects of IL-13 antagonism on in vitro response to allergen, the fold change differences in the presence and absence of antagonist are calculated by determining the difference in the log 2 frequency. Raw P-values are adjusted for multiplicity according to the false discovery rate (FDR) procedure of Benjamini and Hochberg (Reiner (2003) Bioinformatics, 19:368-75) using Spotfire (Somerville, Mass.).

### Identification of Genes Modulated by IL-13

Sdf Human monocytes are purified from PBMC of 5 individual subjects and cultured in the presence or absence of IL-13. Cells are harvested at 2, 6, 12 and 24 hours and gene expression levels are assessed by Affymetrix U95A chip. Genes with an IL-13 dependent difference with an FDR<0.05 and an IL-13 dependent fold change of at least 1.5 fold at any time point are considered to be significantly modulated by IL-13

### INCORPORATION BY REFERENCE

All publications and patent documents and all GenBank records corresponding to sequence accession numbers cited in this application are incorporated by reference in their entirety as they exist on the filing date of this application for all purposes to the same extent as if the contents of each individual publication, patent document, or GenBank record was incorporated herein.

TABLE 1a
PROBESETS DETERMINED TO BE ASSOCIATED WITH ASTHMA
AND/OR IL-13 RESPONSIVENESS

| | | | D | | | | |
|---|---|---|---|---|---|---|---|
| | | | Raw P | | | | |
| | | | value' | | | | |
| | | | Assocation | | | | |
| | | | with | E | | | |
| A | | | Asthma, all | log2_diff_all | | | |
| NetAffx- | B | C | patients, all | aos_all | F | G | H |
| GeneSymbol | Gene Description | QUALIFIER | time points | hvos | ‘AOS_intra_subject_cv_sgnl | HVOS_intra_subject_cv_sgnl | Basis for selection |
| NRG1 | neuregulin 1 | 206343_s_at | 2.56E−04 | 0.286 | 38.08 | 41.44 | passes all filters for asthma |
| | | | | | | | and IL13 |
| FCER2 | Fc fragment of IgE, low affinity II, receptor | 206759_at | 4.13E−02 | 0.184 | 64.45 | 69.41 | poor consistency, but known |
| | for (CD23A) | | | | | | IL13 relationship |
| LDLR | low density lipoprotein receptor (familial | 202068_s_at | 7.80E−07 | −0.203 | 34.69 | 35.08 | passes all filters for asthma |
| | hypercholesterolemia) | | | | | | and IL13, and severity related |
| PRPF39 | gb: NM_018333.1 /DEF = Homo sapiens | 220553_s_at | 9.52E−29 | −0.335 | 29.04 | 29.77 | asthma p value, CV, |
| | hypothetical protein FLJ11128 (FLJ11128), | | | | | | FC, severity |
| | mRNA. /FEA = mRNA /GEN = FLJ11128 | | | | | | |
| | /PROD = hypothetical protein FLJ11128 | | | | | | |
| | /DB_XREF = gi: 8922887 /UG = Hs.250477 | | | | | | |
| | hypothetical protein FLJ11128 | | | | | | |
| | /FL = gb: NM_018333.1 | | | | | | |
| CCNL2 /// | cyclin L2 | 221427_s_at | 1.56E−22 | −0.346 | 29.98 | 30.74 | Based on asthma P-value, CV, |
| LOC643556 | | | | | | | severity and FC, no IL13 filter. |
| EIF2AK3 | eukaryotic translation initiation factor 2- | 218696_at | 1.29E−28 | −0.391 | 35.34 | 28.75 | Based on asthma P-value, CV, |
| | alpha kinase 3 | | | | | | severity and FC, no IL13 filter. |
| NUP88 | nucleoporin 88 kDa | 202900_s_at | 3.07E−28 | −0.337 | 27.95 | 25.17 | Based on asthma P-value, CV, |
| | | | | | | | severity and FC, no IL13 filter. |
| SCML1 | sex comb on midleg-like 1 (Drosophila) | 218793_s_at | 3.18E−21 | −0.448 | 40.58 | 34.82 | Based on asthma P-value, CV, |
| | | | | | | | severity and FC, no IL13 filter. |
| TNPO1 | Transportin 1 | 212635_at | 4.82E−26 | −0.328 | 29.27 | 25.42 | Based on asthma P-value, CV, |
| | | | | | | | severity and FC, no IL13 filter. |
| NR4A3 | nuclear receptor subfamily 4, group A, | 209959_at | 1.12E−14 | −0.650 | 89.51 | 71.94 | consistency, FC and severity |
| | member 3 | | | | | | |
| ZNF217 | zinc finger protein 217 | 203739_at | 3.39E−09 | −0.174 | 33.41 | 28.85 | IL13, consistency, severity |
| AHR | aryl hydrocarbon receptor | 202820_at | 1.07E−20 | −0.453 | 47.44 | 41.64 | passes all filters for asthma |
| | | | | | | | and IL13 |
| C6orf62 | Chromosome 6 open reading frame 62 | 222309_at | 8.36E−16 | −0.316 | 44.67 | 34.27 | passes all filters for asthma |
| | | | | | | | and IL13 and severity |
| CD69 | CD69 antigen (p60, early T-cell activation | 209795_at | 1.57E−11 | −0.202 | 30.52 | 30.19 | passes all filters for asthma |
| | antigen) | | | | | | and IL13 and severity |
| CD83 | CD83 antigen (activated B lymphocytes, | 204440_at | 3.93E−10 | −0.254 | 40.23 | 36.47 | passes all filters for asthma |
| | immunoglobulin superfamily) | | | | | | and IL13 and severity |
| CNOT8 | CCR4-NOT transcription complex, subunit 8 | 202163_s_at | 3.60E−09 | −0.207 | 36.92 | 32.68 | passes all filters for asthma |
| | | | | | | | and IL13 and severity |
| CSE1L | CSE1 chromosome segregation 1-like | 210766_s_at | 2.02E−20 | −0.264 | 27.14 | 26.80 | passes all filters for asthma |
| | (yeast) | | | | | | and IL13 and severity |
| DUSP10 | dual specificity phosphatase 10 | 215501_s_at | 2.05E−07 | −0.210 | 43.69 | 38.96 | passes all filters for asthma |
| | | | | | | | and IL13 and severity |
| DUSP10 | Dual specificity phosphatase 10 | 221563_at | 4.81E−12 | −0.210 | 33.40 | 28.22 | passes all filters for asthma |
| | | | | | | | and IL13 |
| EIF1AX | Eukaryotic translation initiation factor 1A, X- | 201016_at | 3.18E−15 | −0.273 | 36.78 | 35.49 | passes all filters for asthma |
| | linked | | | | | | and IL13 |
| HSPC111 | hypothetical protein HSPC111 | 203023_at | 7.77E−13 | −0.209 | 32.50 | 29.53 | passes all filters for asthma |
| | | | | | | | and IL13 |
| IRF1 | interferon regulatory factor 1 | 202531_at | 2.22E−09 | −0.249 | 31.94 | 35.12 | passes all filters for asthma |
| | | | | | | | and IL13 |
| ITPR1 | inositol 1,4,5-triphosphate receptor, type 1 | 216944_s_at | 1.33E−11 | −0.276 | 41.14 | 36.10 | passes all filters for asthma |
| | | | | | | | and IL13 |
| KLF9 | Kruppel-like factor 9 | 203543_s_at | 5.91E−12 | −0.314 | 46.54 | 41.26 | passes all filters for asthma |
| | | | | | | | and IL13 |
| MAFF | Cluster Incl. AL021977: bK447C4.1 (novel | 36711_at | 6.11E−11 | −0.342 | 45.72 | 36.76 | passes all filters for asthma |
| | MAFF (v-maf musculoaponeurotic | | | | | | and IL13 |
| | fibrosarcoma (avian) oncogene family, | | | | | | |
| | protein F) LIKE protein) /cds = (0.494) | | | | | | |
| | /gb = AL021977 /gi = 4914526 /ug = Hs.51305 | | | | | | |
| | /len = 2128 | | | | | | |
| MTF2 | likely ortholog of mouse metal response | 203347_s_at | 1.22E−11 | −0.260 | 40.35 | 37.17 | passes all filters for asthma |
| | element binding transcription factor 2 | | | | | | and IL13 |
| NRIP1 | nuclear receptor interacting protein 1 | 202599_s_at | 2.21E−12 | −0.324 | 44.54 | 45.26 | passes all filters for asthma |
| | | | | | | | and IL13 |
| PFDN4 | Prefoldin 4 | 205361_s_at | 5.15E−12 | −0.207 | 33.27 | 29.10 | passes all filters for asthma |
| | | | | | | | and IL13 |
| RAN | RAN, member RAS oncogene family | 200749_at | 3.99E−17 | −0.290 | 31.89 | 29.10 | passes all filters for asthma |
| | | | | | | | and IL13 |
| SFPQ | Splicing factor proline/glutamine rich | 201585_s_at | 5.67E−21 | −0.249 | 28.52 | 26.93 | passes all filters for asthma |
| | (polypyrimidine tract binding protein | | | | | | and IL13 |
| | associated) | | | | | | |
| SMAD7 | SMAD, mothers against DPP homolog 7 | 204790_at | 1.71E−15 | −0.294 | 33.79 | 31.16 | passes all filters for asthma |
| | (Drosophila) | | | | | | and IL13 |
| STCH | Stress 70 protein chaperone, microsome- | 202557_at | 6.48E−17 | −0.309 | 38.43 | 33.34 | passes all filters for asthma |
| | associated, 60 kDa | | | | | | and IL13 |
| SUMO1 | SMT3 suppressor of mif two 3 homolog 1 | 208762_at | 6.96E−15 | −0.299 | 43.61 | 42.26 | passes all filters for asthma |
| | (yeast) | | | | | | and IL13 |
| TIMM17A | translocase of inner mitochondrial | 201821_s_at | 5.63E−21 | −0.260 | 31.53 | 31.20 | passes all filters for asthma |
| | membrane 17 homolog A (yeast) | | | | | | and IL13 |
| TNFAIP3 | Tumor necrosis factor, alpha-induced | 202643_s_at | 2.62E−10 | −0.231 | 35.66 | 30.37 | passes all filters for asthma |
| | protein 3 | | | | | | and IL13 |
| FUSIP1 /// | gb: NM_021993.1 /DEF = Homo sapiens | 204299_at | 2.18E−31 | −0.427 | 42.30 | 28.93 | passes all filters for asthma |
| LOC642558 | TLS-associated serine-arginine protein 2 | | | | | | and IL13 |
| | (TASR2), mRNA. /FEA = mRNA | | | | | | |
| | /GEN = TASR2 /PROD = TLS-associated | | | | | | |
| | serine-arginine protein 2 | | | | | | |
| | /DB_XREF = gi: 12056475 /UG = Hs.3530 | | | | | | |
| | TLS-associated serine-arginine protein 2 | | | | | | |
| | /FL = gb: NM_021993.1 gb: BC005039.1 | | | | | | |
| | gb: AF067730.1 | | | | | | |
| FUSIP1 /// | FUS interacting protein (serine-arginine | 206095_s_at | 1.25E−28 | −0.308 | 27.66 | 23.62 | passes all filters for asthma |
| LOC642558 | rich) 1 | | | | | | and IL13 |
| FBXL11 | Consensus includes RC gb: BE675843 | 208988_at | 4.60E−38 | −0.325 | 23.73 | 20.85 | asthma p value, CV, FC |
| | /FEA = EST /DB_XREF = gi: 10036384 | | | | | | |
| | /DB_XREF = est: 7f17b04.x1 | | | | | | |
| | /CLONE = IMAGE: 3294895 /UG = Hs.219614 | | | | | | |
| | f-box and leucine-rich repeat protein 11 | | | | | | |
| | /FL = gb: AF179221.1 | | | | | | |
| MED6 | mediator of RNA polymerase II | 207078_at | 7.84E−26 | −0.629 | 52.37 | 49.36 | asthma p value, FC |
| | transcription, subunit 6 homolog (yeast) | | | | | | |
| C1orf9 | chromosome 1 open reading frame 9 | 203429_s_at | 1.53E−50 | −0.525 | 38.18 | 38.50 | asthma p value, FC, CV |
| ARMC8 | armadillo repeat containing 8 | 219094_at | 8.08E−28 | −0.319 | 32.01 | 26.70 | Based on asthma P-value, CV, |
| | | | | | | | severity and FC, no IL13 filter. |
| BMS1L | BMS1-like, ribosome assembly protein | 203082_at | 8.29E−32 | −0.319 | 25.01 | 24.84 | Based on asthma P-value, CV, |
| | (yeast) | | | | | | severity and FC, no IL13 filter. |
| BTG3 | BTG family, member 3 | 205548_s_at | 5.38E−33 | −0.332 | 32.03 | 23.01 | Based on asthma P-value, CV, |
| | | | | | | | severity and FC, no IL13 filter. |
| CAND1 | TBP-interacting protein | 207483_s_at | 1.91E−33 | −0.359 | 28.74 | 26.51 | Based on asthma P-value, CV, |
| | | | | | | | severity and FC, no IL13 filter. |
| CCNT2 | Cyclin T2 | 213743_at | 5.92E−28 | −0.393 | 33.44 | 30.38 | Based on asthma P-value, CV, |
| | | | | | | | severity and FC, no IL13 filter. |
| CRSP6 | cofactor required for Sp1 transcriptional | 221517_s_at | 4.41E−41 | −0.467 | 34.46 | 31.16 | Based on asthma P-value, CV, |
| | activation, subunit 6, 77 kDa | | | | | | severity and FC, no IL13 filter. |
| CYLD | Cylindromatosis (turban tumor syndrome) | 60084_at | 2.70E−35 | −0.406 | 35.44 | 30.41 | Based on asthma P-value, CV, |
| | | | | | | | severity and FC, no IL13 filter. |
| DBF4 | activator of S phase kinase | 204244_s_at | 1.53E−35 | −0.447 | 39.45 | 33.28 | Based on asthma P-value, CV, |
| | | | | | | | severity and FC, no IL13 filter. |
| DDX47 | DEAD (Asp-Glu-Ala-Asp) box polypeptide | 220890_s_at | 5.29E−28 | −0.310 | 26.79 | 27.78 | Based on asthma P-value, CV, |
| | 47 | | | | | | severity and FC, no IL13 filter. |
| EZH2 | enhancer of zeste homolog 2 (Drosophila) | 203358_s_at | 1.07E−45 | −0.549 | 40.52 | 38.56 | Based on asthma P-value, CV, |
| | | | | | | | severity and FC, no IL13 filter. |
| FAM98A | DKFZP564F0522 protein | 212333_at | 6.39E−27 | −0.332 | 29.18 | 28.95 | Based on asthma P-value, CV, |
| | | | | | | | severity and FC, no IL13 filter. |
| FBXL11 | F-box and leucine-rich repeat protein 11 | 208989_s_at | 6.24E−37 | −0.349 | 29.13 | 25.74 | Based on asthma P-value, CV, |
| | | | | | | | severity and FC, no IL13 filter. |
| FBXO3 | F-box protein 3 | 218432_at | 1.21E−28 | −0.425 | 33.91 | 30.49 | Based on asthma P-value, CV, |
| | | | | | | | severity and FC, no IL13 filter. |
| HIPK1 | Homeodomain interacting protein kinase 1 | 212293_at | 1.53E−34 | −0.333 | 29.78 | 25.49 | Based on asthma P-value, CV, |
| | | | | | | | severity and FC, no IL13 filter. |
| HSF2 | heat shock transcription factor 2 | 209657_s_at | 2.02E−32 | −0.468 | 39.43 | 31.53 | Based on asthma P-value, CV, |
| | | | | | | | severity and FC, no IL13 filter. |
| PDE4D | phosphodiesterase 4D, cAMP-specific | 210837_s_at | 3.49E−26 | −0.412 | 35.74 | 32.43 | Based on asthma P-value, CV, |
| | (phosphodiesterase E3 dunce homolog, | | | | | | severity and FC, no IL13 filter. |
| | Drosophila) | | | | | | |
| PIGA | phosphatidylinositol glycan, class A | 205281_s_at | 4.79E−28 | −0.327 | 34.06 | 25.83 | Based on asthma P-value, CV, |
| | (paroxysmal nocturnal hemoglobinuria) | | | | | | severity and FC, no IL13 filter. |
| PRDM2 | PR domain containing 2, with ZNF domain | 203057_s_at | 4.13E−31 | −0.311 | 25.95 | 25.16 | Based on asthma P-value, CV, |
| | | | | | | | severity and FC, no IL13 filter. |
| RANBP2 | RAN binding protein 2 | 201713_s_at | 1.44E−35 | −0.512 | 43.59 | 36.15 | Based on asthma P-value, CV, |
| | | | | | | | severity and FC, no IL13 filter. |
| RFC1 | gb: L14922.1 /DEF = Homo sapiens DNA- | 209085_x_at | 3.94E−37 | −0.334 | 26.34 | 25.66 | Based on asthma P-value, CV, |
| | binding protein (PO-GA) mRNA, complete | | | | | | severity and FC, no IL13 filter. |
| | cds. /FEA = mRNA /PROD = DNA-binding | | | | | | |
| | protein /DB_XREF = gi: 307337 | | | | | | |
| | /UG = Hs.166563 replication factor C | | | | | | |
| | (activator 1) 1 (145 kD) /FL = gb: AF040250.1 | | | | | | |
| | gb: L14922.1 | | | | | | |
| RRN3 | RRN3 RNA polymerase I transcription | 222204_s_at | 1.19E−32 | −0.382 | 34.69 | 29.94 | Based on asthma P-value, CV, |
| | factor homolog (yeast) | | | | | | severity and FC, no IL13 filter. |
| SFRS12 | Splicing factor, arginine/serine-rich 12 | 212721_at | 8.02E−53 | −0.422 | 29.12 | 26.63 | Based on asthma P-value, CV, |
| | | | | | | | severity and FC, no IL13 filter. |
| SR140 | U2-associated SR140 protein | 212060_at | 9.71E−38 | −0.392 | 29.68 | 29.59 | Based on asthma P-value, CV, |
| | | | | | | | severity and FC, no IL13 filter. |
| TCERG1 | transcription elongation regulator 1 | 202396_at | 2.05E−39 | −0.404 | 30.46 | 28.30 | Based on asthma P-value, CV, |
| | | | | | | | severity and FC, no IL13 filter. |
| Unknown | Homo sapiens, clone IMAGE: 4214654, | 213158_at | 1.87E−35 | −0.461 | 35.37 | 35.40 | Based on asthma P-value, CV, |
| | mRNA | | | | | | severity and FC, no IL13 filter. |
| ZNF278 | zinc finger protein 278 | 209431_s_at | 6.89E−24 | −0.321 | 28.54 | 27.46 | Based on asthma P-value, CV, |
| | | | | | | | severity and FC, no IL13 filter. |
| ZRF1 | Zuotin related factor 1 | 213097_s_at | 1.21E−39 | −0.391 | 31.35 | 29.61 | Based on asthma P-value, CV, |
| | | | | | | | severity and FC, no IL13 filter. |
| PIAS1 | Protein inhibitor of activated STAT, 1 | 222371_at | 1.20E−48 | −0.800 | 61.25 | 58.80 | consistency and fold change |
| ATP13A3 | ATPase family homolog up-regulated in | 212297_at | 2.58E−32 | −0.380 | 33.83 | 32.12 | passes all filters for asthma |
| | senescence cells | | | | | | and IL19 |
| CLK1 | CDC-like kinase 1 | 214683_s_at | 1.03E−30 | −0.312 | 32.45 | 27.55 | passes all filters for asthma |
| | | | | | | | and IL13 |
| CYP51A1 | cytochrome P450, family 51, subfamily A, | 202314_at | 1.64E−23 | −0.340 | 35.28 | 33.52 | passes all filters for asthma |
| | polypeptide 1 | | | | | | and IL13 |
| JAG1 | jagged 1 (Alagille syndrome) | 209099_x_at | 2.44E−25 | −0.434 | 37.62 | 41.87 | passes all filters for asthma |
| | | | | | | | and IL13 |
| JAG1 | jagged 1 (Alagille syndrome) | 216268_s_at | 8.21E−21 | −0.395 | 38.71 | 41.53 | passes all filters for asthma |
| | | | | | | | and IL13 |
| MEF2D | MADS box transcription enhancer factor 2, | 203003_at | 4.52E−21 | −0.314 | 35.29 | 30.84 | passes all filters for asthma |
| | polypeptide D (myocyte enhancer factor 2D) | | | | | | and IL13 |
| UTP18 | CGI-48 protein | 203721_s_at | 2.87E−43 | −0.329 | 20.93 | 24.93 | passes all filters for asthma |
| | | | | | | | and IL13 |
| ACSL3 | acyl-CoA synthetase long-chain family | 201662_s_at | 3.11E−42 | −0.461 | 35.06 | 33.58 | passes all filters for asthma |
| | member 3 | | | | | | and IL13 |
| C4orf15 | chromosome 4 open reading frame 15 | 210054_at | 7.05E−32 | −0.386 | 32.95 | 30.73 | passes all filters for asthma |
| | | | | | | | and IL13 |
| CLASP2 | Cytoplasmic linker associated protein 2 | 212306_at | 5.38E−48 | −0.370 | 27.82 | 26.60 | passes all filters for asthma |
| | | | | | | | and IL13 |
| GARNL1 | GTPase activating Rap/RanGAP domain- | 213049_at | 1.67E−26 | −0.312 | 30.27 | 27.95 | passes all filters for asthma |
| | like 1 | | | | | | and IL13 |
| IL6ST | Interleukin 6 signal transducer (gp130, | 212195_at | 1.90E−28 | −0.410 | 31.07 | 26.31 | passes all filters for asthma |
| | oncostatin M receptor) | | | | | | and IL13 |
| KIAA1109 | KIAA1109 | 212779_at | 8.04E−31 | −0.336 | 30.34 | 29.23 | passes all filters for asthma |
| | | | | | | | and IL13 |
| SFPQ | Splicing factor proline/glutamine rich | 214016_s_at | 1.83E−47 | −0.401 | 31.30 | 28.04 | passes all filters for asthma |
| | (polypyrimidine tract binding protein | | | | | | and IL13 |
| | associated) | | | | | | |
| SFPQ | Splicing factor proline/glutamine rich | 221768_at | 3.28E−41 | −0.380 | 30.87 | 26.96 | passes all filters for asthma |
| | (polypyrimidine tract binding protein | | | | | | and IL13 |
| | associated) | | | | | | |
| ZBTB11 | zinc finger and BTB domain containing 11 | 204847_at | 7.02E−59 | −0.393 | 26.61 | 23.54 | passes all filters for asthma |
| | | | | | | | and IL13 |
| ANXA4 | annexin A4 | 201301_s_at | 2.20E−06 | 0.509 | 104.44 | 134.54 | asthma p value, FC, severity |
| CEACAM8 | carcinoembryonic antigen-related cell | 206676_at | 9.65E−08 | 0.814 | 84.33 | 86.47 | borderline signal, but FC, |
| | adhesion molecule 8 | | | | | | up, and severity |
| DEFA1 /// | defensin, alpha 1, myeloid-related | 205033_s_at | 9.31E−09 | 0.857 | 72.57 | 63.60 | consistency, FC and severity |
| DEFA3 /// | sequence | | | | | | |
| LOC653600 | | | | | | | |
| ELA2 | elastase 2, neutrophil | 206871_at | 2.75E−08 | 0.715 | 74.41 | 67.80 | consistency, FC, up severity |
| | | | | | | | and function |
| LTF /// | lactotransferrin | 202018_s_at | 2.27E−08 | 0.978 | 90.47 | 86.30 | consistency, upFC and |
| LOC643349 | | | | | | | severity |
| ASGR1 | asialoglycoprotein receptor 1 | 206743_s_at | 9.85E−08 | 0.288 | 37.88 | 43.31 | passes all filters for asthma |
| | | | | | | | and IL13 and severity |
| CSF3R | colony stimulating factor 3 receptor | 203591_s_at | 5.74E−08 | 0.228 | 40.88 | 34.35 | passes all filters for asthma |
| | (granulocyte) | | | | | | and IL13 |
| MYL9 | myosin, light polypeptide 9, regulatory | 201058_s_at | 1.97E−06 | 0.767 | 77.65 | 73.75 | consistency and fold change |
| TNFSF13 /// | tumor necrosis factor (ligand) superfamily, | 209500_x_at | 3.67E−06 | 0.178 | 35.74 | 40.37 | IL13, consistency |
| TNFSF12- | member 13 | | | | | | |
| TNFSF13 | | | | | | | |
| CAT | catalase | 211922_s_at | 6.53E−25 | 0.385 | 33.79 | 37.64 | Based on asthma P-value, CV, |
| | | | | | | | severity and FC, no IL13 filter. |
| FCGR2C | Fc fragment of IgG, low affinity IIc, receptor | 210992_x_at | 9.21E−26 | 0.419 | 32.46 | 32.83 | Based on asthma P-value, CV, |
| | for (CD32) | | | | | | severity and FC, no IL13 filter. |
| MXD1 | MAX dimerization protein 1 | 206877_at | 4.35E−24 | 0.392 | 37.66 | 31.00 | Based on asthma P-value, CV, |
| | | | | | | | severity and FC, no IL13 filter. |
| S100A11 | S100 calcium binding protein A11 | 200660_at | 1.32E−27 | 0.526 | 43.32 | 40.81 | Based on asthma P-value, CV, |
| | (calgizzarin) | | | | | | severity and FC, no IL13 filter. |
| IL1R2 | interleukin 1 receptor, type II | 205403_at | 2.74E−10 | 0.639 | 85.44 | 74.25 | conistency, severity and |
| | | | | | | | function |
| IL1R2 | interleukin 1 receptor, type II | 211372_s_at | 4.59E−12 | 0.684 | 85.63 | 75.01 | conistency, FC, up, severity |
| IL32 | natural killer cell transcript 4 | 203828_s_at | 2.97E−10 | 0.610 | 73.37 | 84.42 | consistency, FC, up, severity |
| CAMP | cathelicidin antimicrobial peptide | 210244_at | 5.30E−11 | 0.873 | 72.48 | 79.60 | consistency and FC, severity |
| CD24 | Consensus includes gb: AK000168.1 | 216379_x_at | 3.38E−14 | 0.701 | 51.51 | 45.10 | consistency and FC and |
| | /DEF = Homo sapiens cDNA FLJ20161 fis, | | | | | | severity |
| | clone COL09252, highly similar to L33930 | | | | | | |
| | Homo sapiens CD24 signal transducer | | | | | | |
| | mRNA. /FEA = mRNA | | | | | | |
| | /DB_XREF = gi: 7020079 /UG = Hs.332045 | | | | | | |
| | Homo sapiens cDNA FLJ20161 fis, clone | | | | | | |
| | COL09252, highly similar to L33930 Homo | | | | | | |
| | sapiens CD24 signal transducer mRNA | | | | | | |
| S100P | S100 calcium binding protein P | 204351_at | 2.09E−12 | 0.760 | 57.03 | 51.49 | consistency and FC and |
| | | | | | | | severity |
| IL8RB | interleukin 8 receptor, beta | 207008_at | 2.86E−14 | 0.579 | 69.75 | 63.04 | consistency, fairFC, up |
| | | | | | | | severity |
| MS4A3 | membrane-spanning 4-domains, subfamily | 210254_at | 1.19E−12 | 0.603 | 54.63 | 53.08 | consistency, FC and severity |
| | A, member 3 (hematopoietic cell-specific) | | | | | | |
| CD24 | CD24 antigen (small cell lung carcinoma | 208651_x_at | 6.08E−12 | 0.688 | 64.51 | 65.54 | consistency, FC and up, |
| | cluster 4 antigen) | | | | | | severity |
| DEFA4 | defensin, alpha 4, corticostatin | 207269_at | 1.25E−12 | 0.768 | 57.06 | 51.82 | consistency, FC and |
| | | | | | | | up, severity |
| GLIPR1 | HIV-1 rev binding protein 2 | 214085_x_at | 9.28E−28 | 0.629 | 65.77 | 60.19 | consistency, FC, severity |
| CLC | Charcot-Leyden crystal protein | 206207_at | 2.31E−21 | 0.768 | 61.19 | 53.10 | consistency, FC, up and |
| | | | | | | | severity |
| VNN3 | vanin 3 | 220528_at | 7.00E−17 | 0.635 | 64.73 | 63.04 | consistency, FC, up, severity |
| FCAR | Fc fragment of IgA, receptor for | 211307_s_at | 9.33E−14 | 0.616 | 76.81 | 89.62 | consistency, FC, up, severity |
| CD24 | CD24 antigen (small cell lung carcinoma | 209771_x_at | 3.78E−11 | 0.680 | 57.56 | 51.90 | consistency, FC, severity |
| | cluster 4 antigen) | | | | | | |
| FCGR3B | Fc fragment of IgG, low affinity IIIb, receptor | 204007_at | 3.72E−13 | 0.637 | 77.64 | 69.14 | IL13 and consistency and FC |
| | for (CD16) | | | | | | and severity |
| CHI3L1 | chitinase 3-like 1 (cartilage glycoprotein-39) | 209396_s_at | 3.28E−19 | 0.888 | 76.40 | 75.36 | IL13 antag in vivo and |
| | | | | | | | consistency and FC and |
| | | | | | | | severity |
| FCN1 | ficolin (collagen/fibrinogen domain | 205237_at | 3.23E−08 | 0.196 | 25.21 | 35.11 | IL13 antagin vivo and |
| | containing) 1 | | | | | | consistency |
| ARG1 | arginase, liver | 206177_s_at | 4.73E−09 | 0.450 | 54.22 | 50.62 | IL13 in vivo mouse |
| LCN2 | lipocalin 2 (oncogene 24p3) | 212531_at | 2.10E−09 | 0.528 | 44.33 | 40.50 | IL13 in vivo mouse, |
| | | | | | | | consistency |
| BLVRA | Biliverdin reductase A | 203771_s_at | 2.78E−18 | 0.296 | 29.32 | 34.52 | passes all filters for asthma |
| | | | | | | | and IL13 and in vivo |
| AK2 | Adenylate kinase 2 | 212175_s_at | 1.38E−12 | 0.203 | 26.80 | 29.28 | passes all filters for asthma |
| | | | | | | | and IL13 |
| ALDOC | aldolase C, fructose-bisphosphate | 202022_at | 1.76E−06 | 0.213 | 35.24 | 49.25 | passes all filters for asthma |
| | | | | | | | and IL13 |
| CD163 | CD163 antigen | 203645_s_at | 2.23E−09 | 0.348 | 49.36 | 58.14 | passes all filters for asthma |
| | | | | | | | and IL13 |
| CD163 | CD163 antigen | 215049_x_at | 1.19E−11 | 0.380 | 48.61 | 54.64 | passes all filters for asthma |
| | | | | | | | and IL13 |
| CDA | cytidine deaminase | 205627_at | 1.16E−17 | 0.393 | 36.26 | 33.02 | passes all filters for asthma |
| | | | | | | | and IL13 |
| CTSC | cathepsin C | 201487_at | 6.79E−17 | 0.319 | 31.82 | 36.87 | passes all filters for asthma |
| | | | | | | | and IL13 |
| GLRX | glutaredoxin (thioltransferase) | 206662_at | 5.26E−08 | 0.259 | 34.84 | 32.61 | passes all filters for asthma |
| | | | | | | | and IL13 |
| GRN | granulin | 211284_s_at | 1.58E−08 | 0.210 | 32.42 | 36.80 | passes all filters for asthma |
| | | | | | | | and IL13 |
| GRN | granulin | 216041_x_at | 2.25E−09 | 0.225 | 34.24 | 36.76 | passes all filters for asthma |
| | | | | | | | and IL13 |
| IL13RA1 | interleukin 13 receptor, alpha 1 | 210904_s_at | 4.58E−21 | 0.345 | 39.27 | 37.32 | passes all filters for asthma |
| | | | | | | | and IL13 |
| LILRB2 /// | leukocyte immunoglobulin-like receptor, | 210784_x_at | 2.44E−06 | 0.208 | 43.92 | 39.69 | passes all filters for asthma |
| LILRB3 | subfamily B (with TM and ITIM domains), | | | | | | and IL13 |
| | member 3 | | | | | | |
| NCF4 | neutrophil cytosolic factor 4, 40 kDa | 205147_x_at | 5.65E−23 | 0.371 | 37.70 | 31.27 | passes all filters for asthma |
| | | | | | | | and IL13 |
| NCF4 | neutrophil cytosolic factor 4, 40 kDa | 207677_s_at | 4.20E−18 | 0.422 | 45.83 | 38.94 | passes all filters for asthma |
| | | | | | | | and IL13 |
| NUP62 | nucleoporin 62 kDa | 207740_s_at | 1.08E−09 | 0.237 | 40.59 | 41.92 | passes all filters for asthma |
| | | | | | | | and IL13 |
| PADI2 | Consensus includes gb: AL049569 | 209791_at | 2.99E−09 | 0.298 | 36.89 | 38.70 | passes all filters for asthma |
| | /DEF = Human DNA sequence from clone | | | | | | and IL13 |
| | RP1-37C10 on chromosome 1p35.2-35.21. | | | | | | |
| | Contains the gene for the ortholog of | | | | | | |
| | mouse and rat PDI (protein-arginine | | | | | | |
| | deiminase (KIAA0994, EC 3.5.3.15, | | | | | | |
| | peptidylarginine deiminase)), the SDHB | | | | | | |
| | gene for succinate dehydrogenase... | | | | | | |
| | /FEA = mRNA_4 /DB_XREF = gi: 5263031 | | | | | | |
| | /UG = Hs.33455 peptidyl arginine deiminase | | | | | | |
| | type II /FL = gb: AB030176.1 | | | | | | |
| RNASE2 | ribonuclease, RNase A family, 2 (liver, | 206111_at | 5.44E−19 | 0.491 | 40.55 | 46.94 | passes all filters for asthma |
| | eosinophil-derived neurotoxin) | | | | | | and IL13 |
| S100A9 | S100 calcium binding protein A9 | 203535_at | 2.43E−17 | 0.339 | 32.52 | 54.23 | passes all filters for asthma |
| | (calgranulin B) | | | | | | and IL13 |
| SCCPDH | CGI-49 protein | 201825_s_at | 1.98E−13 | 0.281 | 32.64 | 32.88 | passes all filters for asthma |
| | | | | | | | and IL13 |
| SELL | selectin L (lymphocyte adhesion molecule | 204563_at | 1.24E−17 | 0.360 | 34.03 | 40.16 | passes all filters for asthma |
| | 1) | | | | | | and IL13 |
| SELPLG | Selectin P ligand | 209879_at | 1.39E−13 | 0.353 | 45.30 | 45.72 | passes all filters for asthma |
| | | | | | | | and IL13 |
| TALDO1 | transaldolase 1 | 201463_s_at | 9.10E−10 | 0.250 | 37.54 | 42.83 | passes all filters for asthma |
| | | | | | | | and IL13 |
| VNN2 | vanin 2 | 205922_at | 6.32E−19 | 0.632 | 48.53 | 50.21 | passes all filters for asthma |
| | | | | | | | and IL13, and severity related |
| FCGR2A | Fc fragment of IgG, low affinity IIa, receptor | 203561_at | 1.48E−26 | 0.444 | 34.87 | 33.95 | passes all filters for asthma |
| | for (CD32) | | | | | | and IL13 |
| PECAM1 | platelet/endothelial cell adhesion molecule | 208983_s_at | 9.48E−21 | 0.372 | 32.47 | 38.27 | passes all filters for asthma |
| | (CD31 antigen) | | | | | | and IL13 |
| CHI3L1 | chitinase 3-like 1 (cartilage glycoprotein-39) | 209395_at | 2.90E−16 | 1.165 | 120.28 | 103.42 | up, other probesetIL13 and |
| | | | | | | | consistency and FC and |
| | | | | | | | severity |
| SPCS2 /// | KIAA0102 gene product | 201239_s_at | 3.42E−36 | 0.339 | 26.97 | 30.96 | asthma p value, CV, FC |
| LOC653566 | | | | | | | |
| CCR2 | chemokine (C—C motif) receptor 2 | 206978_at | 6.47E−25 | 0.336 | 32.02 | 28.40 | Based on asthma P-value, CV, |
| | | | | | | | severity and FC, no IL13 filter. |
| FCGR2C | Fc fragment of IgG, low affinity IIc, receptor | 211395_x_at | 3.04E−31 | 0.383 | 29.35 | 30.36 | Based on asthma P-value, CV, |
| | for (CD32) | | | | | | severity and FC, no IL13 filter. |
| FPR1 | formyl peptide receptor 1 | 205119_s_at | 1.23E−30 | 0.604 | 42.64 | 42.35 | Based on asthma P-value, CV, |
| | | | | | | | severity and FC, no IL13 filter. |
| FRAT2 | frequently rearranged in advanced T-cell | 209864_at | 1.87E−31 | 0.293 | 27.89 | 20.88 | Based on asthma P-value, CV, |
| | lymphomas 2 | | | | | | severity and FC, no IL13 filter. |
| LYN | v-yes-1 Yamaguchi sarcoma viral related | 202626_s_at | 3.53E−34 | 0.348 | 30.00 | 25.45 | Based on asthma P-value, CV, |
| | oncogene homolog | | | | | | severity and FC, no IL13 filter. |
| LYN | v-yes-1 Yamaguchi sarcoma viral related | 210754_s_at | 1.38E−26 | 0.306 | 31.31 | 26.93 | Based on asthma P-value, CV, |
| | oncogene homolog | | | | | | severity and FC, no IL13 filter. |
| MNDA | myeloid cell nuclear differentiation antigen | 204959_at | 5.34E−29 | 0.560 | 49.72 | 40.90 | Based on asthma P-value, CV, |
| | | | | | | | severity and FC, no IL13 filter. |
| RNF13 | ring finger protein 13 | 201779_s_at | 2.94E−36 | 0.410 | 33.67 | 34.76 | Based on asthma P-value, CV, |
| | | | | | | | severity and FC, no IL13 filter. |
| SP110 | SP110 nuclear body protein | 208012_x_at | 2.60E−41 | 0.410 | 27.65 | 22.99 | Based on asthma P-value, CV, |
| | | | | | | | severity and FC, no IL13 filter. |
| SP110 | SP110 nuclear body protein | 209761_s_at | 5.49E−40 | 0.453 | 41.78 | 31.91 | Based on asthma P-value, CV, |
| | | | | | | | severity and FC, no IL13 filter. |
| SP110 | SP110 nuclear body protein | 209762_x_at | 4.86E−31 | 0.326 | 24.83 | 22.65 | Based on asthma P-value, CV, |
| | | | | | | | severity and FC, no IL13 filter. |
| TLR8 | toll-like receptor 8 | 220832_at | 1.96E−20 | 0.832 | 82.03 | 84.24 | FC, low frequency, but up |
| | | | | | | | gene |
| ANP32A | Acidic (leucine-rich) nuclear phosphoprotein | 201051_at | 2.56E−55 | 0.368 | 24.34 | 23.83 | IL13, consistency, low CV, |
| | 32 family, member A | | | | | | severity |
| BASP1 | brain abundant, membrane attached signal | 202391_at | 3.23E−23 | 0.506 | 43.55 | 47.35 | passes all filters for asthma |
| | protein 1 | | | | | | and IL13 |
| GAB2 | GRB2-associated binding protein 2 | 203853_s_at | 8.99E−10 | 0.234 | 35.59 | 42.25 | passes all filters for asthma |
| | | | | | | | and IL13 |
| PICALM | Phosphatidylinositol binding clathrin | 215236_s_at | 7.18E−26 | 0.471 | 49.17 | 49.66 | passes all filters for asthma |
| | assembly protein | | | | | | and IL13 |
| PRKAR1A | protein kinase, cAMP-dependent, | 200604_s_at | 5.40E−20 | 0.300 | 40.73 | 42.48 | passes all filters for asthma |
| | regulatory, type I, alpha (tissue specific | | | | | | and IL13 |
| | extinguisher 1) | | | | | | |
| TNFSF10 | tumor necrosis factor (ligand) superfamily, | 202688_at | 1.13E−19 | 0.411 | 40.59 | 35.21 | passes all filters for asthma |
| | member 10 | | | | | | and IL13 |
| ACTR2 | Consensus includes gb: BE566290 | 200728_at | 2.27E−31 | 0.404 | 30.62 | 35.52 | passes all filters for asthma |
| | /FEA = EST /DB_XREF = gi: 9810010 | | | | | | and IL13 |
| | /DB_XREF = est: 601339864F1 | | | | | | |
| | /CLONE = IMAGE: 3682406 /UG = Hs.42915 | | | | | | |
| | ARP2 (actin-related protein 2, yeast) | | | | | | |
| | homolog /FL = gb: AF006082.1 | | | | | | |
| | gb: NM_005722.1 | | | | | | |
| CD14 | CD14 antigen | 201743_at | 4.49E−26 | 0.473 | 32.47 | 39.08 | passes all filters for asthma |
| | | | | | | | and IL13 |
| GLRX | glutaredoxin (thioltransferase) | 209276_s_at | 5.19E−31 | 0.307 | 24.56 | 22.39 | passes all filters for asthma |
| | | | | | | | and IL13 |
| LAMP2 | lysosomal-associated membrane protein 2 | 203041_s_at | 4.36E−25 | 0.344 | 29.74 | 29.16 | passes all filters for asthma |
| | | | | | | | and IL13 |
| TNFSF10 | tumor necrosis factor (ligand) superfamily, | 202687_s_at | 2.38E−21 | 0.408 | 44.12 | 36.33 | passes all filters for asthma |
| | member 10 | | | | | | and IL13 |
| IL21R | interleukin 21 receptor | 221658_s_at | 0.00058494 | −0.183 | 51.72 | 54.25 | severity (best) |

TABLE 1b
PROBESETS DETERMINED TO BE ASSOCIATED WITH ASTHMA
AND/OR IL-13 RESPONSIVENESS

| | | C | | |
|---|---|---|---|---|
| | | Meets all AOS FC, | D | |
| | B | FDR, AND % CV | study_fdr_p | |
| A | Signal | filters AND meets | all visitAOSv | |
| NetAffx- | Average | in vitro IL13 FC | all visit | E |
| GeneSymbol | AOS | and FDR filters | HVOS | hvos_v_severity_pattern_fdr_0001 |
| NRG1 | 70.93300288 | yes | 0.000649679 | --- |
| FCER2 | 26.87524999 | failed at least one | 0.06545711 | --- |
| | | asthma or IL13 | | |
| | | filter | | |
| LDLR | 124.1549244 | yes | 3.14441E−06 | --h |
| PRPF39 | 114.0589725 | failed at least one | 0 | -hh |
| | | asthma or IL13 | | |
| | | filter | | |
| CCNL2 /// | 123.4261618 | failed at least one | 0 | -hh |
| LOC643556 | | asthma or IL13 | | |
| | | filter | | |
| EIF2AK3 | 274.44087 | failed at least one | 0 | -hh |
| | | asthma or IL13 | | |
| | | filter | | |
| NUP88 | 167.8477839 | failed at least one | 0 | -hh |
| | | asthma or IL13 | | |
| | | filter | | |
| SCML1 | 71.55604311 | failed at least one | 0 | -hh |
| | | asthma or IL13 | | |
| | | filter | | |
| TNPO1 | 126.6087483 | failed at least one | 0 | -hh |
| | | asthma or IL13 | | |
| | | filter | | |
| NR4A3 | 45.02028618 | failed at least one | 1.44434E−13 | -hh |
| | | asthma or IL13 | | |
| | | filter | | |
| ZNF217 | 146.4744833 | failed at least one | 1.99574E−08 | -hh |
| | | asthma or IL13 | | |
| | | filter | | |
| AHR | 98.31079541 | yes | 0 | -hh |
| C6orf62 | 109.2188141 | yes | 1.34623E−14 | -hh |
| CD69 | 911.3149763 | yes | 1.29848E−10 | -hh |
| CD83 | 633.482763 | yes | 2.62877E−09 | -hh |
| CNOT8 | 168.5195236 | yes | 2.10199E−08 | -hh |
| CSE1L | 85.80572746 | yes | 0 | -hh |
| DUSP10 | 108.001544 | yes | 9.0426E−07 | -hh |
| DUSP10 | 226.1691671 | yes | 4.2957E−11 | -hh |
| EIF1AX | 52.24429519 | yes | 4.38116E−14 | -hh |
| HSPC111 | 35.70482621 | yes | 7.72704E−12 | -hh |
| IRF1 | 805.2855493 | yes | 1.34992E−08 | -hh |
| ITPR1 | 33.51806115 | yes | 1.11754E−10 | -hh |
| KLF9 | 169.0090347 | yes | 5.19615E−11 | -hh |
| MAFF | 427.3750827 | yes | 4.64609E−10 | -hh |
| MTF2 | 40.05407156 | yes | 1.03466E−10 | -hh |
| NRIP1 | 279.1013755 | yes | 2.0734E−11 | -hh |
| PFDN4 | 133.4805559 | yes | 4.58381E−11 | -hh |
| RAN | 104.1539098 | yes | 0 | -hh |
| SFPQ | 249.7976202 | yes | 0 | -hh |
| SMAD7 | 117.0530449 | yes | 2.59456E−14 | -hh |
| STCH | 100.0569238 | yes | 0 | -hh |
| SUMO1 | 36.66593466 | yes | 9.56653E−14 | -hh |
| TIMM17A | 156.2467863 | yes | 0 | -hh |
| TNFAIP3 | 1119.072385 | yes | 1.80104E−09 | -hh |
| FUSIP1 /// | 155.348343 | yes | 0 | -hh |
| LOC642558 | | | | |
| FUSIP1 /// | 222.650046 | yes | 0 | -hh |
| LOC642558 | | | | |
| FBXL11 | 130.9670047 | failed at least one | 0 | hhh |
| | | asthma or IL13 | | |
| | | filter | | |
| MED6 | 50.39713359 | failed at least one | 0 | hhh |
| | | asthma or IL13 | | |
| | | filter | | |
| C1orf9 | 160.1458915 | failed at least one | 0 | hhh |
| | | asthma or IL13 | | |
| | | filter | | |
| ARMC8 | 75.76836596 | failed at least one | 0 | hhh |
| | | asthma or IL13 | | |
| | | filter | | |
| BMS1L | 109.8496904 | failed at least one | 0 | hhh |
| | | asthma or IL13 | | |
| | | filter | | |
| BTG3 | 274.7712677 | failed at least one | 0 | hhh |
| | | asthma or IL13 | | |
| | | filter | | |
| CAND1 | 136.9822478 | failed at least one | 0 | hhh |
| | | asthma or IL13 | | |
| | | filter | | |
| CCNT2 | 43.81108228 | failed at least one | 0 | hhh |
| | | asthma or IL13 | | |
| | | filter | | |
| CRSP6 | 144.7730278 | failed at least one | 0 | hhh |
| | | asthma or IL13 | | |
| | | filter | | |
| CYLD | 108.1450109 | failed at least one | 0 | hhh |
| | | asthma or IL13 | | |
| | | filter | | |
| DBF4 | 170.4515211 | failed at least one | 0 | hhh |
| | | asthma or IL13 | | |
| | | filter | | |
| DDX47 | 637.6182135 | failed at least one | 0 | hhh |
| | | asthma or IL13 | | |
| | | filter | | |
| EZH2 | 50.51582676 | failed at least one | 0 | hhh |
| | | asthma or IL13 | | |
| | | filter | | |
| FAM98A | 72.3406249 | failed at least one | 0 | hhh |
| | | asthma or IL13 | | |
| | | filter | | |
| FBXL11 | 173.6063009 | failed at least one | 0 | hhh |
| | | asthma or IL13 | | |
| | | filter | | |
| FBXO3 | 66.90527513 | failed at least one | 0 | hhh |
| | | asthma or IL13 | | |
| | | filter | | |
| HIPK1 | 263.5480876 | failed at least one | 0 | hhh |
| | | asthma or IL13 | | |
| | | filter | | |
| HSF2 | 135.5690337 | failed at least one | 0 | hhh |
| | | asthma or IL13 | | |
| | | filter | | |
| PDE4D | 60.69084458 | failed at least one | 0 | hhh |
| | | asthma or IL13 | | |
| | | filter | | |
| PIGA | 112.539613 | failed at least one | 0 | hhh |
| | | asthma or IL13 | | |
| | | filter | | |
| PRDM2 | 369.0891854 | failed at least one | 0 | hhh |
| | | asthma or IL13 | | |
| | | filter | | |
| RANBP2 | 281.7290261 | failed at least one | 0 | hhh |
| | | asthma or IL13 | | |
| | | filter | | |
| ZRF1 | 308.3033989 | failed at least one | 0 | hhh |
| | | asthma or IL13 | | |
| | | filter | | |
| PIAS1 | 46.64045427 | failed at least one | 0 | hhh |
| | | asthma or IL13 | | |
| | | filter | | |
| ATP13A3 | 69.05182433 | yes | 0 | hhh |
| CLK1 | 554.3180327 | yes | 0 | hhh |
| CYP51A1 | 35.58447706 | yes | 0 | hhh |
| JAG1 | 52.35423155 | yes | 0 | hhh |
| JAG1 | 38.58639535 | yes | 0 | hhh |
| MEF2D | 84.45262915 | yes | 0 | hhh |
| UTP18 | 244.8115999 | yes | 0 | hhh |
| ACSL3 | 104.5698957 | yes | 0 | hhh |
| C4orf15 | 291.2957602 | yes | 0 | hhh |
| CLASP2 | 70.73554407 | yes | 0 | hhh |
| GARNL1 | 79.83075306 | yes | 0 | hhh |
| IL6ST | 351.1599925 | yes | 0 | hhh |
| KIAA1109 | 139.7158703 | yes | 0 | hhh |
| SFPQ | 524.3217781 | yes | 0 | hhh |
| SFPQ | 327.4194028 | yes | 0 | hhh |
| ZBTB11 | 220.8998228 | yes | 0 | hhh |
| ANXA4 | 110.2394672 | failed at least one | 8.19608E−06 | --l |
| | | asthma or IL13 | | |
| | | filter | | |
| CEACAM8 | 36.34296886 | failed at least one | 4.48732E−07 | --l |
| | | asthma or IL13 | | |
| | | filter | | |
| DEFA1 /// | 1175.323077 | failed at least one | 5.11661E−08 | --l |
| DEFA3 /// | | asthma or IL13 | | |
| LOC653600 | | filter | | |
| ELA2 | 34.68204765 | failed at least one | 1.40865E−07 | --l |
| | | asthma or IL13 | | |
| | | filter | | |
| LTF /// | 211.8884353 | failed at least one | 1.18233E−07 | --l |
| LOC643349 | | asthma or IL13 | | |
| | | filter | | |
| ASGR1 | 55.02315435 | yes | 4.57147E−07 | --l |
| CSF3R | 295.6427996 | yes | 2.7782E−07 | --l |
| MYL9 | 47.6688663 | failed at least one | 7.40877E−06 | -l- |
| | | asthma or IL13 | | |
| | | filter | | |
| TNFSF13 /// | 454.185498 | failed at least one | 1.31712E−05 | -l- |
| TNFSF12-TNFSF13 | | asthma or IL13 | | |
| | | filter | | |
| CAT | 151.5761608 | failed at least one | 0 | -ll |
| | | asthma or IL13 | | |
| | | filter | | |
| FCGR2C | 211.2085751 | failed at least one | 0 | -ll |
| | | asthma or IL13 | | |
| | | filter | | |
| MXD1 | 133.5450473 | failed at least one | 0 | -ll |
| | | asthma or IL13 | | |
| | | filter | | |
| S100A11 | 419.4411835 | failed at least one | 0 | -ll |
| | | asthma or IL13 | | |
| | | filter | | |
| IL1R2 | 51.98941393 | failed at least one | 1.87328E−09 | -ll |
| | | asthma or IL13 | | |
| | | filter | | |
| IL1R2 | 34.43463449 | failed at least one | 4.10453E−11 | -ll |
| | | asthma or IL13 | | |
| | | filter | | |
| IL32 | 165.4247612 | failed at least one | 2.01811E−09 | -ll |
| | | asthma or IL13 | | |
| | | filter | | |
| CAMP | 122.0248158 | failed at least one | 4.07475E−10 | -ll |
| | | asthma or IL13 | | |
| | | filter | | |
| CD24 | 87.57466892 | failed at least one | 4.11845E−13 | -ll |
| | | asthma or IL13 | | |
| | | filter | | |
| S100P | 221.9623254 | failed at least one | 1.96217E−11 | -ll |
| | | asthma or IL13 | | |
| | | filter | | |
| IL8RB | 45.97242255 | failed at least one | 3.51263E−13 | -ll |
| | | asthma or IL13 | | |
| | | filter | | |
| MS4A3 | 64.9910355 | failed at least one | 1.16187E−11 | -ll |
| | | asthma or IL13 | | |
| | | filter | | |
| CD24 | 39.04164933 | failed at least one | 5.33724E−11 | -ll |
| | | asthma or IL13 | | |
| | | filter | | |
| DEFA4 | 116.1741062 | failed at least one | 1.21758E−11 | -ll |
| | | asthma or IL13 | | |
| | | filter | | |
| GLIPR1 | 177.7234204 | failed at least one | 0 | -ll |
| | | asthma or IL13 | | |
| | | filter | | |
| CLC | 156.0192656 | failed at least one | 0 | -ll |
| | | asthma or IL13 | | |
| | | filter | | |
| VNN3 | 37.23126106 | failed at least one | 0 | -ll |
| | | asthma or IL13 | | |
| | | filter | | |
| FCAR | 51.84024762 | failed at least one | 1.07153E−12 | -ll |
| | | asthma or IL13 | | |
| | | filter | | |
| CD24 | 86.70160845 | failed at least one | 2.95714E−10 | -ll |
| | | asthma or IL13 | | |
| | | filter | | |
| FCGR3B | 412.1004734 | failed at least one | 3.86956E−12 | -ll |
| | | asthma or IL13 | | |
| | | filter | | |
| CHI3L1 | 37.29455996 | failed at least one | 0 | -ll |
| | | asthma or IL13 | | |
| | | filter | | |
| FCN1 | 2828.646474 | failed at least one | 1.63677E−07 | -ll |
| | | asthma or IL13 | | |
| | | filter | | |
| ARG1 | 30.85997161 | failed at least one | 2.70705E−08 | -ll |
| | | asthma or IL13 | | |
| | | filter | | |
| LCN2 | 156.1940446 | failed at least one | 1.28002E−08 | -ll |
| | | asthma or IL13 | | |
| | | filter | | |
| BLVRA | 78.04713527 | yes | 0 | -ll |
| AK2 | 89.80666112 | yes | 1.33283E−11 | -ll |
| ALDOC | 45.80153849 | yes | 6.64418E−06 | -ll |
| CD163 | 285.7250965 | yes | 1.35109E−08 | -ll |
| CD163 | 286.8658725 | yes | 1.01101E−10 | -ll |
| CDA | 128.1966577 | yes | 0 | -ll |
| CTSC | 272.5656885 | yes | 0 | -ll |
| GLRX | 691.1995447 | yes | 2.56432E−07 | -ll |
| GRN | 368.175537 | yes | 8.3996E−08 | -ll |
| GRN | 862.8613246 | yes | 1.36513E−08 | -ll |
| IL13RA1 | 91.58111953 | yes | 0 | -ll |
| LILRB2 /// | 157.9679806 | yes | 8.98859E−06 | -ll |
| LILRB3 | | | | |
| NCF4 | 191.650321 | yes | 0 | -ll |
| NCF4 | 171.5688728 | yes | 0 | -ll |
| NUP62 | 41.26447806 | yes | 6.86144E−09 | -ll |
| PADI2 | 117.6038028 | yes | 1.77242E−08 | -ll |
| RNASE2 | 226.9375796 | yes | 0 | -ll |
| S100A9 | 4869.24767 | yes | 0 | -ll |
| SCCPDH | 48.40147644 | yes | 2.16228E−12 | -ll |
| SELL | 1193.083165 | yes | 0 | -ll |
| SELPLG | 246.0277891 | yes | 1.55991E−12 | -ll |
| TALDO1 | 923.4822475 | yes | 5.81893E−09 | -ll |
| VNN2 | 273.6878605 | yes | 0 | -ll |
| FCGR2A | 367.4858084 | yes | 0 | -ll |
| PECAM1 | 235.4143414 | yes | 0 | -ll |
| CHI3L1 | 13.30867662 | failed at least one | 6.87162E−15 | -ll |
| | | asthma or IL13 | | |
| | | filter | | |
| SPCS2 /// | 197.588944 | failed at least one | 0 | lll |
| LOC653566 | | asthma or IL13 | | |
| | | filter | | |
| CCR2 | 62.19685451 | failed at least one | 0 | lll |
| | | asthma or IL13 | | |
| | | filter | | |
| FCGR2C | 320.0024338 | failed at least one | 0 | lll |
| | | asthma or IL13 | | |
| | | filter | | |
| FPR1 | 637.7236886 | failed at least one | 0 | lll |
| | | asthma or IL13 | | |
| | | filter | | |
| FRAT2 | 86.67753359 | failed at least one | 0 | lll |
| | | asthma or IL13 | | |
| | | filter | | |
| LYN | 668.5004752 | failed at least one | 0 | lll |
| | | asthma or IL13 | | |
| | | filter | | |
| LYN | 799.5990504 | failed at least one | 0 | lll |
| | | asthma or IL13 | | |
| | | filter | | |
| MNDA | 441.9118025 | failed at least one | 0 | lll |
| | | asthma or IL13 | | |
| | | filter | | |
| RNF13 | 264.2967848 | failed at least one | 0 | lll |
| | | asthma or IL13 | | |
| | | filter | | |
| SP110 | 250.2803795 | failed at least one | 0 | lll |
| | | asthma or IL13 | | |
| | | filter | | |
| SP110 | 142.1184803 | failed at least one | 0 | lll |
| | | asthma or IL13 | | |
| | | filter | | |
| SP110 | 258.454744 | failed at least one | 0 | lll |
| | | asthma or IL13 | | |
| | | filter | | |
| TLR8 | 26.51766876 | failed at least one | 0 | lll |
| | | asthma or IL13 | | |
| | | filter | | |
| ANP32A | 525.7486516 | failed at least one | 0 | lll |
| | | asthma or IL13 | | |
| | | filter | | |
| BASP1 | 721.6199711 | yes | 0 | lll |
| GAB2 | 263.3492369 | yes | 5.7646E−09 | lll |
| PICALM | 97.21388876 | yes | 0 | lll |
| PRKAR1A | 92.84858327 | yes | 0 | lll |
| TNFSF10 | 200.7840535 | yes | 0 | lll |
| ACTR2 | 750.1160614 | yes | 0 | lll |
| CD14 | 1113.798421 | yes | 0 | lll |
| GLRX | 467.6519696 | yes | 0 | lll |
| LAMP2 | 235.3305667 | yes | 0 | lll |
| TNFSF10 | 83.75964069 | yes | 0 | lll |
| IL21R | 54.31207645 | No | 0.001388531 | --h |

| A | | | |
|---|---|---|---|
| NetAffx- | F | G | H |
| GeneSymbol | hvos_v_moderate_fdr_p | hvos_v_severe_fdr_p | abs_fold_diff_hvos_mild |
| NRG1 | 0.009026698 | 0.000548332 | 1.0900313 |
| FCER2 | 0.010150025 | 0.588595289 | 1.1964585 |
| LDLR | 0.000378886 | 2.65709E−05 | 1.20027 |
| PRPF39 | 0 | 0 | 1.1247868 |
| CCNL2 /// | 0 | 0 | 1.2255175 |
| LOC643556 | | | |
| EIF2AK3 | 0 | 0 | 1.1863362 |
| NUP88 | 0 | 0 | 1.1709633 |
| SCML1 | 2.4324E−14 | 0 | 1.2840292 |
| TNPO1 | 0 | 0 | 1.186311 |
| NR4A3 | 1.63103E−10 | 1.54924E−11 | 1.4022462 |
| ZNF217 | 5.17555E−06 | 7.04239E−08 | 1.0980946 |
| AHR | 0 | 0 | 1.2893531 |
| C6orf62 | 1.81592E−12 | 1.20134E−12 | 1.1136811 |
| CD69 | 5.83459E−08 | 9.93597E−09 | 1.1487626 |
| CD83 | 2.89789E−06 | 2.99302E−08 | 1.200123 |
| CNOT8 | 5.36053E−08 | 1.83283E−06 | 1.0796458 |
| CSE1L | 1.27411E−14 | 0 | 1.1135737 |
| DUSP10 | 3.34595E−05 | 1.77714E−05 | 1.1679209 |
| DUSP10 | 3.61919E−08 | 5.96659E−10 | 1.1181729 |
| EIF1AX | 8.46698E−11 | 3.80603E−12 | 1.1563988 |
| HSPC111 | 2.70276E−09 | 6.7388E−10 | 1.1273993 |
| IRF1 | 1.76774E−06 | 5.21722E−07 | 1.1792367 |
| ITPR1 | 2.97293E−08 | 2.10408E−09 | 1.1346336 |
| KLF9 | 6.3076E−10 | 3.09248E−08 | 1.1699081 |
| MAFF | 6.95822E−08 | 9.40399E−08 | 1.2628265 |
| MTF2 | 9.16693E−09 | 2.80316E−09 | 1.1115951 |
| NRIP1 | 8.81562E−08 | 1.17789E−10 | 1.2045342 |
| PFDN4 | 2.56116E−08 | 4.7911E−10 | 1.1042632 |
| RAN | 6.21824E−12 | 1.61403E−13 | 1.1695185 |
| SFPQ | 0 | 0 | 1.1578352 |
| SMAD7 | 4.72846E−09 | 4.67674E−13 | 1.2365496 |
| STCH | 2.91383E−12 | 6.93844E−13 | 1.1914706 |
| SUMO1 | 4.56007E−11 | 6.83317E−12 | 1.1507182 |
| TIMM17A | 2.4324E−14 | 0 | 1.1479441 |
| TNFAIP3 | 7.0278E−07 | 1.48444E−07 | 1.2039276 |
| FUSIP1 /// | 0 | 0 | 1.2015814 |
| LOC642558 | | | |
| FUSIP1 /// | 0 | 0 | 1.1688886 |
| LOC642558 | | | |
| FBXL11 | 0 | 0 | 1.2043693 |
| MED6 | 0 | 0 | 1.5056512 |
| C1orf9 | 0 | 0 | 1.3716135 |
| ARMC8 | 0 | 0 | 1.1863882 |
| BMS1L | 0 | 0 | 1.2105861 |
| BTG3 | 0 | 0 | 1.2478516 |
| CAND1 | 0 | 0 | 1.1882618 |
| CCNT2 | 0 | 0 | 1.229274 |
| CRSP6 | 0 | 0 | 1.3028652 |
| CYLD | 0 | 0 | 1.2465837 |
| DBF4 | 0 | 0 | 1.2468562 |
| DDX47 | 0 | 0 | 1.2021934 |
| EZH2 | 0 | 0 | 1.3826625 |
| FAM98A | 0 | 0 | 1.2384189 |
| FBXL11 | 0 | 0 | 1.2118986 |
| FBXO3 | 0 | 0 | 1.2472924 |
| HIPK1 | 0 | 0 | 1.1752893 |
| HSF2 | 0 | 0 | 1.3326057 |
| PDE4D | 0 | 0 | 1.3689426 |
| PIGA | 0 | 0 | 1.2340365 |
| PRDM2 | 0 | 0 | 1.1782245 |
| RANBP2 | 0 | 0 | 1.3514163 |
| ZRF1 | 0 | 0 | 1.2347722 |
| PIAS1 | 0 | 0 | 1.5428585 |
| ATP13A3 | 0 | 0 | 1.2798182 |
| CLK1 | 0 | 0 | 1.2048727 |
| CYP51A1 | 0 | 0 | 1.2587317 |
| JAG1 | 0 | 0 | 1.3641315 |
| JAG1 | 0 | 2.28955E−12 | 1.2761916 |
| MEF2D | 0 | 5.07631E−14 | 1.3088095 |
| UTP18 | 0 | 0 | 1.223598 |
| ACSL3 | 0 | 0 | 1.4218835 |
| C4orf15 | 0 | 0 | 1.2324241 |
| CLASP2 | 0 | 0 | 1.2674452 |
| GARNL1 | 0 | 0 | 1.1879682 |
| IL6ST | 0 | 0 | 1.2633429 |
| KIAA1109 | 0 | 0 | 1.2342201 |
| SFPQ | 0 | 0 | 1.2385865 |
| SFPQ | 0 | 0 | 1.2334359 |
| ZBTB11 | 0 | 0 | 1.2637228 |
| ANXA4 | 0.000233882 | 4.79227E−06 | 1.1309154 |
| CEACAM8 | 0.000634477 | 9.15327E−08 | 1.519524 |
| DEFA1 /// | 0.000397424 | 3.26872E−09 | 1.5338402 |
| DEFA3 /// | | | |
| LOC653600 | | | |
| ELA2 | 0.000326398 | 6.32029E−09 | 1.2542478 |
| LTF /// | 0.000226279 | 8.31872E−08 | 1.8379633 |
| LOC643349 | | | |
| ASGR1 | 0.000148335 | 1.20674E−06 | 1.2065426 |
| CSF3R | 0.000690523 | 3.09701E−09 | 1.0695103 |
| MYL9 | 3.58705E−06 | 0.002573408 | 1.7379649 |
| TNFSF13 /// | 2.28221E−05 | 0.000313891 | 1.0807806 |
| TNFSF12-TNFSF13 | | | |
| CAT | 0 | 0 | 1.2280046 |
| FCGR2C | 0 | 0 | 1.2545678 |
| MXD1 | 2.4324E−14 | 0 | 1.1968959 |
| S100A11 | 0 | 0 | 1.2915987 |
| IL1R2 | 6.72792E−06 | 3.51911E−09 | 1.4720202 |
| IL1R2 | 1.02892E−06 | 3.55039E−11 | 1.4842882 |
| IL32 | 1.17884E−07 | 6.04317E−09 | 1.11549 |
| CAMP | 1.35642E−05 | 1.03263E−10 | 1.6363391 |
| CD24 | 7.59375E−08 | 7.43807E−14 | 1.3288694 |
| S100P | 1.75198E−06 | 3.14763E−12 | 1.432839 |
| IL8RB | 1.23946E−08 | 1.02503E−12 | 1.3613348 |
| MS4A3 | 1.2841E−06 | 4.67674E−13 | 1.2675646 |
| CD24 | 1.12175E−06 | 1.2905E−11 | 1.3310676 |
| DEFA4 | 4.80167E−06 | 8.85173E−14 | 1.3464402 |
| GLIPR1 | 0 | 0 | 1.3329401 |
| CLC | 1.87349E−12 | 0 | 1.6124318 |
| VNN3 | 4.37432E−11 | 6.66307E−14 | 1.4115476 |
| FCAR | 4.0301E−09 | 7.18789E−12 | 1.3220409 |
| CD24 | 2.05983E−06 | 6.47498E−11 | 1.2482704 |
| FCGR3B | 5.99908E−08 | 1.33606E−11 | 1.4145095 |
| CHI3L1 | 4.47903E−11 | 0 | 1.6458281 |
| FCN1 | 4.28563E−06 | 7.44476E−07 | 1.0577687 |
| ARG1 | 1.81913E−05 | 5.70196E−09 | 1.1107325 |
| LCN2 | 3.93274E−05 | 1.55783E−09 | 1.1884576 |
| BLVRA | 6.75951E−14 | 7.05435E−13 | 1.194241 |
| AK2 | 3.38967E−09 | 1.04803E−09 | 1.1106102 |
| ALDOC | 2.46228E−05 | 6.00654E−05 | 1.0690178 |
| CD163 | 1.21096E−06 | 1.40139E−08 | 1.0705831 |
| CD163 | 1.24863E−07 | 4.65491E−11 | 1.1066695 |
| CDA | 2.09458E−10 | 0 | 1.1438888 |
| CTSC | 9.75121E−12 | 7.05435E−13 | 1.220479 |
| GLRX | 9.68991E−05 | 1.62209E−06 | 1.2201218 |
| GRN | 5.17523E−06 | 5.38134E−07 | 1.0955708 |
| GRN | 7.71937E−07 | 2.7227E−07 | 1.1173798 |
| IL13RA1 | 1.19315E−10 | 0 | 1.1967039 |
| LILRB2 /// | 4.91485E−05 | 1.01913E−05 | 1.0042018 |
| LILRB3 | | | |
| NCF4 | 2.12032E−13 | 0 | 1.1911238 |
| NCF4 | 2.12996E−10 | 0 | 1.2252915 |
| NUP62 | 3.64897E−07 | 1.43901E−07 | 1.1265323 |
| PADI2 | 8.83879E−06 | 5.30958E−09 | 1.0728412 |
| RNASE2 | 1.26533E−13 | 0 | 1.0803996 |
| S100A9 | 4.01028E−11 | 0 | 1.172443 |
| SCCPDH | 1.06914E−09 | 1.91953E−10 | 1.1491969 |
| SELL | 1.67365E−12 | 4.26396E−14 | 1.1866791 |
| SELPLG | 1.2491E−10 | 1.5163E−09 | 1.2267512 |
| TALDO1 | 9.97646E−06 | 5.71439E−10 | 1.081436 |
| VNN2 | 1.00833E−11 | 0 | 1.48662 |
| FCGR2A | 0 | 0 | 1.2731478 |
| PECAM1 | 0 | 0 | 1.208294 |
| CHI3L1 | 9.92991E−09 | 0 | 2.0208938 |
| SPCS2 /// | 0 | 0 | 1.3018007 |
| LOC653566 | | | |
| CCR2 | 0 | 0 | 1.2069824 |
| FCGR2C | 0 | 0 | 1.2221703 |
| FPR1 | 0 | 0 | 1.5022589 |
| FRAT2 | 0 | 0 | 1.2018121 |
| LYN | 0 | 0 | 1.2766533 |
| LYN | 0 | 0 | 1.2469906 |
| MNDA | 0 | 0 | 1.3873799 |
| RNF13 | 0 | 0 | 1.2997808 |
| SP110 | 0 | 0 | 1.3041581 |
| SP110 | 0 | 0 | 1.3022007 |
| SP110 | 0 | 0 | 1.2274595 |
| TLR8 | 3.58366E−13 | 0 | 1.9588998 |
| ANP32A | 0 | 0 | 1.2897683 |
| BASP1 | 0 | 0 | 1.4065954 |
| GAB2 | 1.67698E−05 | 7.27501E−08 | 1.2551724 |
| PICALM | 0 | 0 | 1.3579167 |
| PRKAR1A | 1.93205E−13 | 0 | 1.2114841 |
| TNFSF10 | 2.40619E−13 | 1.82559E−14 | 1.3524677 |
| ACTR2 | 0 | 0 | 1.3889208 |
| CD14 | 0 | 0 | 1.3995864 |
| GLRX | 0 | 0 | 1.2453596 |
| LAMP2 | 0 | 0 | 1.2291658 |
| TNFSF10 | 0 | 0 | 1.2942577 |
| IL21R | 0.094525341 | 4.70737E−05 | 1.0645965 |

| A | | | K |
|---|---|---|---|
| NetAffx- | I | J | Accessions from |
| GeneSymbol | abs_fold_diff_hvos_moderate | abs_fold_diff_hvos_severe | Affymetrix |
| NRG1 | 1.2009552 | 1.2617001 | NM_004495 /// |
| | | | NM_013956 /// |
| | | | NM_013957 /// |
| | | | NM_013958 /// |
| | | | NM_013959 /// |
| | | | NM_013960 /// |
| | | | NM_013961 /// |
| | | | NM_013962 /// |
| | | | NM_013964 |
| FCER2 | 1.2293845 | 1.0478603 | NM_002002 |
| LDLR | 1.1357137 | 1.1567128 | NM_000527 |
| PRPF39 | 1.2589747 | 1.2907404 | NM_005645 /// |
| | | | NM_017922 |
| CCNL2 /// | 1.2849947 | 1.2663662 | NM_001039577 /// |
| LOC643556 | | | NM_030937 |
| EIF2AK3 | 1.3211942 | 1.3255965 | NM_004836 |
| NUP88 | 1.2503571 | 1.2926015 | NM_002532 |
| SCML1 | 1.3555516 | 1.3865761 | NM_001037535 /// |
| | | | NM_001037536 /// |
| | | | NM_001037540 /// |
| | | | NM_006746 |
| TNPO1 | 1.2503142 | 1.2723927 | NM_002270 /// |
| | | | NM_153188 |
| NR4A3 | 1.5755965 | 1.596894 | NM_006981 /// |
| | | | NM_173198 /// |
| | | | NM_173199 /// |
| | | | NM_173200 |
| ZNF217 | 1.1207241 | 1.1400187 | NM_006526 |
| AHR | 1.3782095 | 1.374457 | NM_001621 |
| C6orf62 | 1.2601851 | 1.2558883 | NM_030939 |
| CD69 | 1.1479189 | 1.1529257 | NM_001781 |
| CD83 | 1.176171 | 1.2054826 | NM_001040280 /// |
| | | | NM_004233 |
| CNOT8 | 1.1751139 | 1.1489444 | NM_004779 |
| CSE1L | 1.2038077 | 1.2141259 | NM_001316 |
| DUSP10 | 1.1552563 | 1.1566265 | NM_007207 /// |
| | | | NM_144728 /// |
| | | | NM_144729 |
| DUSP10 | 1.1522904 | 1.1678452 | NM_007207 /// |
| | | | NM_144728 /// |
| | | | NM_144729 |
| EIF1AX | 1.2082984 | 1.2183392 | NM_001412 |
| HSPC111 | 1.1578531 | 1.1600445 | NM_016391 |
| IRF1 | 1.1869743 | 1.1917444 | NM_002198 |
| ITPR1 | 1.2120579 | 1.2245588 | NM_002222 |
| KLF9 | 1.2691417 | 1.2326508 | NM_001206 |
| MAFF | 1.2736062 | 1.2634372 | NM_012323 /// |
| | | | NM_152878 |
| MTF2 | 1.2049463 | 1.2071854 | NM_007358 |
| NRIP1 | 1.2329314 | 1.2779426 | NM_003489 |
| PFDN4 | 1.1518573 | 1.1664589 | NM_002623 |
| RAN | 1.2214164 | 1.2329798 | NM_006325 |
| SFPQ | 1.1962132 | 1.1872436 | NM_005066 |
| SMAD7 | 1.2019874 | 1.2472938 | NM_005904 |
| STCH | 1.2426809 | 1.2438993 | NM_006948 |
| SUMO1 | 1.235779 | 1.2404618 | NM_001005781 /// |
| | | | NM_001005782 /// |
| | | | NM_003352 |
| TIMM17A | 1.1923635 | 1.212214 | NM_006335 |
| TNFAIP3 | 1.1679942 | 1.1739664 | NM_006290 |
| FUSIP1 /// | 1.3341209 | 1.3822719 | NM_006625 /// |
| LOC642558 | | | NM_054016 |
| FUSIP1 /// | 1.2563506 | 1.2336688 | NM_006625 /// |
| LOC642558 | | | NM_054016 |
| FBXL11 | 1.2222387 | 1.289897 | NM_012308 |
| MED6 | 1.524219 | 1.5755845 | NM_005466 |
| C1orf9 | 1.4490903 | 1.4429596 | NM_014283 /// |
| | | | NM_016227 |
| ARMC8 | 1.2515934 | 1.2550128 | NM_014154 /// |
| | | | NM_015396 /// |
| | | | NM_213654 |
| BMS1L | 1.2476723 | 1.2544633 | NM_014753 |
| BTG3 | 1.2584417 | 1.2619079 | NM_006806 |
| CAND1 | 1.2878968 | 1.2947149 | NM_018448 |
| CCNT2 | 1.3310442 | 1.3128059 | NM_001241 /// |
| | | | NM_058241 |
| CRSP6 | 1.3875687 | 1.3918511 | NM_004268 |
| CYLD | 1.3155581 | 1.3488064 | NM_001042355 /// |
| | | | NM_001042412 /// |
| | | | NM_015247 |
| DBF4 | 1.3739064 | 1.3763653 | NM_006716 |
| DDX47 | 1.2276199 | 1.2570807 | NM_016355 /// |
| | | | NM_201224 |
| EZH2 | 1.4485263 | 1.4911837 | NM_004456 /// |
| | | | NM_152998 |
| FAM98A | 1.2388327 | 1.2815254 | NM_015475 |
| FBXL11 | 1.257824 | 1.3003388 | NM_012308 |
| FBXO3 | 1.3412003 | 1.3609909 | NM_012175 /// |
| | | | NM_033406 |
| HIPK1 | 1.2572686 | 1.2773049 | NM_152696 /// |
| | | | NM_181358 /// |
| | | | NM_198268 /// |
| | | | NM_198269 |
| HSF2 | 1.3814872 | 1.3942687 | NM_004506 |
| PDE4D | 1.3314407 | 1.3225221 | NM_006203 |
| PIGA | 1.2690353 | 1.2450219 | NM_002641 /// |
| | | | NM_020473 |
| PRDM2 | 1.2332903 | 1.2584299 | NM_001007257 /// |
| | | | NM_012231 /// |
| | | | NM_015866 |
| RANBP2 | 1.4235628 | 1.4416865 | NM_006267 |
| ZRF1 | 1.3228078 | 1.3149614 | NM_014377 |
| PIAS1 | 1.7714526 | 1.7529087 | NM_016166 |
| ATP13A3 | 1.2878747 | 1.3175344 | XM_927225 /// |
| | | | XM_931948 /// |
| | | | XM_942079 |
| CLK1 | 1.2488093 | 1.2413915 | NM_001024646 /// |
| | | | NM_004071 |
| CYP51A1 | 1.2718816 | 1.2622854 | NM_000786 |
| JAG1 | 1.3836917 | 1.3197165 | NM_000214 |
| JAG1 | 1.3691005 | 1.2744982 | NM_000214 |
| MEF2D | 1.246638 | 1.2274384 | NM_005920 |
| UTP18 | 1.2564842 | 1.2625439 | NM_016001 |
| ACSL3 | 1.358821 | 1.3849614 | NM_004457 /// |
| | | | NM_203372 |
| C4orf15 | 1.2914104 | 1.335465 | NM_024511 |
| CLASP2 | 1.2909897 | 1.2976883 | NM_015097 |
| GARNL1 | 1.2582785 | 1.235858 | NM_014990 /// |
| | | | NM_194301 |
| IL6ST | 1.3160256 | 1.3523378 | NM_002184 /// |
| | | | NM_175767 |
| KIAA1109 | 1.2664959 | 1.2633508 | XM_371706 /// |
| | | | XM_934076 /// |
| | | | XM_934079 /// |
| | | | XM_934081 /// |
| | | | XM_934084 /// |
| | | | XM_934087 /// |
| | | | XM_934092 /// |
| | | | XM_934095 /// |
| | | | XM_934097 /// |
| | | | XM_936897 /// |
| | | | XM_943047 /// |
| | | | XM_943057 /// |
| | | | XM_943062 /// |
| | | | XM_943070 /// |
| | | | XM_943072 /// |
| | | | XM_943076 /// |
| | | | XM_943084 /// |
| | | | XM_943089 |
| SFPQ | 1.3242047 | 1.3320277 | NM_005066 |
| SFPQ | 1.3098783 | 1.305736 | NM_005066 |
| ZBTB11 | 1.3142959 | 1.3218937 | NM_014415 |
| ANXA4 | 1.4038844 | 1.5037427 | NM_001153 |
| CEACAM8 | 1.5818483 | 1.9866999 | NM_001816 |
| DEFA1 /// | 1.5913513 | 2.0993713 | NM_004084 /// |
| DEFA3 /// | | | NM_005217 |
| LOC653600 | | | |
| ELA2 | 1.4983137 | 1.8696151 | NM_001972 |
| LTF /// | 1.7625597 | 2.2055305 | NM_002343 /// |
| LOC643349 | | | XM_926682 |
| ASGR1 | 1.1958345 | 1.2472404 | NM_001671 |
| CSF3R | 1.1311176 | 1.2284606 | NM_000760 /// |
| | | | NM_156038 /// |
| | | | NM_156039 /// |
| | | | NM_172313 |
| MYL9 | 1.9168758 | 1.5214736 | NM_006097 /// |
| | | | NM_181526 |
| TNFSF13 /// | 1.1496026 | 1.1236226 | NM_003808 /// |
| TNFSF12-TNFSF13 | | | NM_172087 /// |
| | | | NM_172088 /// |
| | | | NM_172089 |
| CAT | 1.2887458 | 1.3367678 | NM_001752 |
| FCGR2C | 1.3337322 | 1.355572 | NM_001005410 /// |
| | | | NM_001005411 /// |
| | | | NM_001005412 /// |
| | | | NM_201563 |
| MXD1 | 1.2797653 | 1.3658903 | NM_002357 |
| S100A11 | 1.4555425 | 1.4541998 | NM_005620 |
| IL1R2 | 1.4810048 | 1.6463968 | NM_004633 /// |
| | | | NM_173343 |
| IL1R2 | 1.5128059 | 1.7217256 | NM_004633 /// |
| | | | NM_173343 |
| IL32 | 1.5466106 | 1.5943075 | NM_001012631 /// |
| | | | NM_001012632 /// |
| | | | NM_001012633 /// |
| | | | NM_001012634 /// |
| | | | NM_001012635 /// |
| | | | NM_001012636 /// |
| | | | NM_001012718 /// |
| | | | NM_004221 |
| CAMP | 1.6527018 | 2.0523546 | NM_004345 |
| CD24 | 1.5329024 | 1.7778519 | NM_013230 |
| S100P | 1.5636109 | 1.8754753 | NM_005980 |
| IL8RB | 1.4455629 | 1.5656198 | NM_001557 |
| MS4A3 | 1.4235672 | 1.6647463 | NM_001031666 /// |
| | | | NM_001031809 /// |
| | | | NM_006138 |
| CD24 | 1.5208683 | 1.7573935 | NM_013230 |
| DEFA4 | 1.532612 | 1.9541149 | NM_001925 |
| GLIPR1 | 1.5426836 | 1.5919833 | NM_006851 |
| CLC | 1.6220217 | 1.7974969 | NM_001828 |
| VNN3 | 1.530229 | 1.6023256 | NM_001024460 /// |
| | | | NM_018399 /// |
| | | | NM_078625 |
| FCAR | 1.5104606 | 1.596433 | NM_002000 /// |
| | | | NM_133269 /// |
| | | | NM_133271 /// |
| | | | NM_133272 /// |
| | | | NM_133273 /// |
| | | | NM_133274 /// |
| | | | NM_133277 /// |
| | | | NM_133278 /// |
| | | | NM_133279 /// |
| | | | NM_133280 |
| CD24 | 1.5247802 | 1.7521099 | NM_013230 |
| FCGR3B | 1.5000236 | 1.6351324 | NM_000570 |
| CHI3L1 | 1.7400788 | 1.9984528 | NM_001276 |
| FCN1 | 1.1503577 | 1.158168 | NM_002003 |
| ARG1 | 1.3295277 | 1.4535418 | NM_000045 |
| LCN2 | 1.3710201 | 1.5615426 | NM_005564 |
| BLVRA | 1.2397541 | 1.2228556 | NM_000712 |
| AK2 | 1.154778 | 1.1560139 | NM_001625 /// |
| | | | NM_013411 |
| ALDOC | 1.1752506 | 1.1620939 | NM_005165 |
| CD163 | 1.2713729 | 1.3139648 | NM_004244 /// |
| | | | NM_203416 |
| CD163 | 1.2854848 | 1.355049 | NM_004244 /// |
| | | | NM_203416 |
| CDA | 1.2805546 | 1.3779105 | NM_001785 |
| CTSC | 1.2456267 | 1.2535174 | NM_001814 /// |
| | | | NM_148170 |
| GLRX | 1.175648 | 1.2123243 | NM_002064 |
| GRN | 1.1563325 | 1.1681712 | NM_001012479 /// |
| | | | NM_002087 |
| GRN | 1.1723434 | 1.1751952 | NM_001012479 /// |
| | | | NM_002087 |
| IL13RA1 | 1.2191561 | 1.3322448 | NM_001560 |
| LILRB2 /// | 1.1658321 | 1.1761444 | NM_005874 /// |
| LILRB3 | | | NM_006864 |
| NCF4 | 1.2600886 | 1.3447754 | NM_000631 /// |
| | | | NM_013416 |
| NCF4 | 1.2980644 | 1.400882 | NM_000631 /// |
| | | | NM_013416 |
| NUP62 | 1.1831131 | 1.1850369 | NM_012346 /// |
| | | | NM_016553 /// |
| | | | NM_153718 /// |
| | | | NM_153719 |
| PADI2 | 1.2120843 | 1.2770249 | NM_007365 |
| RNASE2 | 1.4064658 | 1.470496 | NM_002934 |
| S100A9 | 1.2472153 | 1.299472 | NM_002965 |
| SCCPDH | 1.2195332 | 1.2239605 | NM_016002 |
| SELL | 1.2848616 | 1.2996667 | NM_000655 |
| SELPLG | 1.2968625 | 1.2697532 | NM_003006 |
| TALDO1 | 1.1663666 | 1.232109 | NM_006755 |
| VNN2 | 1.5103815 | 1.5993172 | NM_004665 /// |
| | | | NM_078488 |
| FCGR2A | 1.347988 | 1.3890123 | NM_021642 |
| PECAM1 | 1.3008057 | 1.3048504 | NM_000442 |
| CHI3L1 | 2.0049198 | 2.5315099 | NM_001276 |
| SPCS2 /// | 1.2726261 | 1.2506267 | NM_014752 /// |
| LOC653566 | | | XM_930430 /// |
| | | | XM_934795 /// |
| | | | XM_934796 /// |
| | | | XM_934797 /// |
| | | | XM_940181 /// |
| | | | XM_944484 /// |
| | | | XM_944485 /// |
| | | | XM_944490 |
| CCR2 | 1.2570451 | 1.2766177 | NM_000647 /// |
| | | | NM_000648 |
| FCGR2C | 1.3067098 | 1.3164378 | NM_001005410 /// |
| | | | NM_001005411 /// |
| | | | NM_001005412 /// |
| | | | NM_201563 |
| FPR1 | 1.5140272 | 1.5286369 | NM_002029 |
| FRAT2 | 1.2121175 | 1.241748 | NM_012083 |
| LYN | 1.2474337 | 1.295761 | NM_002350 |
| LYN | 1.2144684 | 1.2545185 | NM_002350 |
| MNDA | 1.4468299 | 1.5162714 | NM_002432 |
| RNF13 | 1.2946395 | 1.3663729 | NM_007282 /// |
| | | | NM_183381 /// |
| | | | NM_183382 /// |
| | | | NM_183383 /// |
| | | | NM_183384 |
| SP110 | 1.3298263 | 1.3315606 | NM_004509 /// |
| | | | NM_004510 /// |
| | | | NM_080424 |
| SP110 | 1.3621926 | 1.3884504 | NM_004509 /// |
| | | | NM_004510 /// |
| | | | NM_080424 |
| SP110 | 1.2673443 | 1.245558 | NM_004509 /// |
| | | | NM_004510 /// |
| | | | NM_080424 |
| TLR8 | 1.7335482 | 1.7922032 | NM_016610 /// |
| | | | NM_138636 |
| ANP32A | 1.2590967 | 1.3192117 | NM_006305 |
| BASP1 | 1.4181785 | 1.4248169 | NM_006317 |
| GAB2 | 1.150799 | 1.1858317 | NM_012296 /// |
| | | | NM_080491 |
| PICALM | 1.3418938 | 1.4326251 | NM_001008660 /// |
| | | | NM_007166 |
| PRKAR1A | 1.2227879 | 1.2427804 | NM_002734 /// |
| | | | NM_212471 /// |
| | | | NM_212472 |
| TNFSF10 | 1.3233824 | 1.3304447 | NM_003810 |
| ACTR2 | 1.3044029 | 1.3278847 | NM_001005386 /// |
| | | | NM_005722 |
| CD14 | 1.3964797 | 1.3791678 | NM_000591 /// |
| | | | NM_001040021 |
| GLRX | 1.2280767 | 1.2432021 | NM_002064 |
| LAMP2 | 1.256374 | 1.2894208 | NM_002294 /// |
| | | | NM_013995 |
| TNFSF10 | 1.3274412 | 1.3317059 | NM_003810 |
| IL21R | 1.0833414 | 1.198681 | NM_021798(11), |
| | | | NM_181078 |
| | | | (11), NM_181079 |
| | | | (11) |

TABLE 2
ANNOTATIONS OF PREVIOUSLY UNCHARACTERIZED MARKERS

| | | C | | | | | | | J | |
|---|---|---|---|---|---|---|---|---|---|---|
| A | B | Affy | | | F | G | H | I | Trans | |
| Affymetrix | Affymetrix | Consensus | D | E | NCBI Gene | Refseq/GenBank | Refseq | Orthologs-Mus | Membrane | K |
| Qualifier | Annotations | Seq Hits to | NCBI-Gene | NCBI-Aliases | Description | Accessions | Protein | & Rat | domains | GO |
| 203429_s_at | C1orf9 | 3′UTR of | C1ORF9 | CH1 | chromosome | NM_016227 | NP_057311 | Variant 1 89% | NP_055098 | None |
| | | NM_016227, | | | 1 open | | (Variant | similaity to Mus | (7-25) | |
| | | NM_014283 | | | reading | | 2), NP_055098.1 | predicted | | |
| | | | | | frame 9 | | (Variant1) | XP_922178 & | | |
| | | | | | protein; | | | 87% similarity to | | |
| | | | | | membrane | | | rat | | |
| | | | | | protein CH1 | | | np_955435.rsrat_aa, | | |
| | | | | | | | | Variant 2 | | |
| | | | | | | | | 83% similaity to | | |
| | | | | | | | | Mus predicted | | |
| | | | | | | | | XP_922178 & | | |
| | | | | | | | | 88% similarity to | | |
| | | | | | | | | rat | | |
| | | | | | | | | np_955435.rsrat_aa | | |
| 210054_at | C4orf15 | 3′UTR of | C4ORF15 | DKFZp686I1868 | hypothetical | NM_024511 | NP_078787, | Percent | No | None |
| | | NM_024511 | | IT1, | protein | | | Similarity: 84.245 | | |
| | | | | MGC4701 | LOC79441 | | | & Percent | | |
| | | | | | | | | Identity: 78.773 | | |
| | | | | | | | | to NR: 109499876 | | |
| | | | | | | | | ref\|XP_001057582.1\| | | |
| | | | | | | | | PREDICTED: | | |
| | | | | | | | | similar to EEA1 | | |
| | | | | | | | | (Early Endosome | | |
| | | | | | | | | Antigen, Rab | | |
| | | | | | | | | effector) homolog | | |
| | | | | | | | | family member | | |
| | | | | | | | | (eea-1) isoform 1 | | |
| | | | | | | | | [Rattus | | |
| | | | | | | | | norvegicus], | | |
| | | | | | | | | Percent | | |
| | | | | | | | | Similarity: 84.386 | | |
| | | | | | | | | & Percent | | |
| | | | | | | | | Identity: 78.070 | | |
| | | | | | | | | to mouse | | |
| | | | | | | | | ortholog | | |
| | | | | | | | | NP_666271 | | |
| 222309_at | C6orf62 | | | | | | | | No | |
| 212779_at | KIAA1109 | ORF and | KIAA1109 | DKFZp781P0474, | fragile site- | DQ335469 | ABC59821 | Percent | 25-47 aa | Molecular |
| | | 3′UTR of | | FSA, | associated | | | Similarity: 97.815 | | Function: |
| | | DQ335469 | | MGC110967 | protein; | | | Percent Identity: | | Aspartic-type |
| | | | | | hypothetical | | | 96.847 to mus | | endopeptidase, |
| | | | | | protein | | | XP_980288 | | Biological |
| | | | | | LOC84162 | | | prediction, 33-39% | | Process: |
| | | | | | | | | similarity to | | proteolysis |
| | | | | | | | | C. elegans | | |
| | | | | | | | | proteins | | |
| | | | | | | | | q8wtl7_caeel.trembl, | | |
| | | | | | | | | q9n3r9_caeel.trembl (lpd-3) | | |
| 213158_at | | predicted | ZBTB20 | HOF; DPZF; | zinc finger | NM_015642 | NP_062752 | Yes in Mus & | No | DNA binding |
| | | 3′UTR of | | ODA-8S; | and BTB | | | well-conserved | | IEA |
| | | ZBTB20 | | ZNF288; | domain | | | | | metal ion |
| | | | | DKFZp566F123 | containing 20 | | | | | binding IEA |
| | | | | | | | | | | protein binding |
| | | | | | | | | | | IEA |
| | | | | | | | | | | zinc ion binding |
| | | | | | | | | | | IEA |
| | | | | | | | | | | Process |
| | | | | | | | | | | Evidence |
| | | | | | | | | | | regulation of |
| | | | | | | | | | | transcription, |
| | | | | | | | | | | DNA- |
| | | | | | | | | | | dependent IEA |
| | | | | | | | | | | transcription |
| | | | | | | | | | | IEA |
| | | | | | | | | | | Component |
| | | | | | | | | | | Evidence |
| | | | | | | | | | | intracellular IEA |
| | | | | | | | | | | nucleus IEA |

TABLE 3
(Allergy Drugs in Development or on the Market)

| MARKETER | BRAND NAME (Generic Name) | MECHANISM |
|---|---|---|
| Schering-Plough | Claritin & Claritin D (loratidine) | Anti-histamine |
| UCB | Vancenase (beclomethasone) | Steroid |
| | Reactine (cetirizine) (US) | Anti-histamine |
| | Zyrtec (cetirizine) (ex US) | |
| | Longifene (buclizine) | Anti-histamine |
| | UCB 28754 (ceterizine alalogue) | Anti-histamine |
| Glaxo | Beconase (beclomethasone) | Steroid |
| | Flonase (fluticasone) | Steroid |
| Aventis | Allegra (fexofenadine) | Anti-histamine |
| | Seldane (terfenadine) | |
| Pfizer | Reactine (cetirizine) (US) | Anti-histamine |
| | Zyrtec/Reactine (cetirizine) (ex US) | |
| | (both licensed from UCB) | |
| Sepracor | Allegra (fexofenadine) | Anti-histamine |
| | Desloratadine (lic to Schering-Plought) | Anti-histamine |
| | Cetirizine (−) (lic to UCB) | Anti-histamine |
| | Norastemizole (option to J&J not exercised, Nov. | |
| | 17, 1999) | |
| B. Ingelheim | Alesion (epinastine) | Anti-histamine |
| Aventis | Kestin (ebastine) (US) | |
| | Bastel (ebastme) Eu/Ger) | |
| | Nasacort (tramcinolone) | Steroid |
| Johnson & Johnson | Hismanol (estemizole) | Anti-histamine |
| | Livostin/Livocarb (levocabastine) | Anti-histamine |
| AstraZeneca | Rhinocort (budesonide) (Astra) | Steroid |
| Merck | Rhmocort (budesonide) | Steroid |
| Eisai | Azeptin (azelastine) | Anti-histamine |
| Kissei | Rizaben (tranilast) | Anti-histamine |
| Shionogi | Triludan (terfenadine) | Anti-histamine |
| | S-5751 | |
| Schwarz | Zolim (mizolastine) | Anti-histamine |
| Daiichi | Zyrtec (cetirizine) (ex US) | Anti-histamine |
| Tanabe | Talion/TAU-284 (betatastine) | Anti-histamine |
| Seiyaku | | |
| Sankyo** | CS 560 (Hypersensitizaion therapy for cedar pollen | Other |
| | allergy) | |
| Asta Medica | Azelastine-MDPI (azelastine) | Anti-histamine |
| BASF | HSR 609 | Anti-histamine |
| SR Pharma | SRL 172 | Immunomodulation |
| Peptide | Allergy vaccine (allergy (hayfever, anaphylaxis, | Downregulates IgE |
| | atopic asthma)) | |
| Therapeutics | Tolerizing peptide vaccine (rye grass peptide (T | Immuno- |
| | cell epitope)) | suppressant |
| Coley | CpG DNA | Immunomodulation |
| Pharmaceutical | | |
| Group | | |
| Genetech | Anti-IgE | Down-regulator of |
| | | IgE |
| SR Pharma | SRL 172 | Immunomodulation |

TABLE 4
(Asthma Drugs in Development or on the Market)

| MARKETER | BRAND NAME (Generic Name) | MECHANISM |
|---|---|---|
| Glaxo | Serevent (salmeterol) | Bronchodilator/beta-2 agonist |
| | Flovent (fluticasone) | Steroid |
| | Flixotide (fluticasone) | |
| | Becotide (betamethasone) | Steroid |
| | Ventolin (salbutamol) | Bronchodilator/beta-2 agonist |
| | Seretide (salmeterol & | Beta agonist & steroid |
| | fluticasone) | |
| | GW215864 | Steroid, hydrolysable |
| | GW250495 | Steroid, hydrolysable |
| | GW28267 | Adenosine A2a receptor |
| | | agonist |
| AstraZeneca | Bambec (bambuterol) (Astra) | |
| | Pulmicort (budesonide) | Steroid |
| | (Astra) | |
| | Bricanyl Turbuhaler | Bronchodilator/beta-2 agonist |
| | (terbutaline) (Astra) | |
| | Accolate (zafurlukast) | Leukotriene antagonist Clo- |
| | (Zeneca) | Phyllin (theophylline) |
| | Inspiryl (salbutamol) (Astra) | Bronchodilator/beta-2 agonist |
| | Oxis Turbuhaler | Bronchodilator/beta-2 agonist |
| | (D2522/formoterol) | |
| | Symbicort (pulmicort-oxis | Steroid |
| | combination) | |
| | Roflepanide (Astra) | Steroid |
| | Bronica (seratrodast) | Thromboxane A2 synthesis |
| | | inhibitor |
| | ZD 4407 (Zeneca) | 5 lipoxygenase inhibitor |
| B. Ingelheim | Atrovent (Ipratropium) | Bronchodilator/anti-cholinergic |
| | Berodual (ipratropium & | Bronchodilator/beta-2 agonist |
| | fenoterol) | |
| | Berotec (fenoterol) | Bronchodilator/beta-2 agonist |
| | Alupent (orciprenaline) | Bronchodilator/beta-2 agonist |
| | Ventilat (oxitropium) | Bronchodilator/anti-cholinergic |
| | Spiropent (clenbuterol) | Bronchodilator/beta-2 agonist |
| | Inhacort (flunisolide) | Steroid |
| | B1679/tiotropium bromide | |
| | RPR 106541 | Steroid |
| | BLIX 1 | Potassium channel |
| | BIIL284 | LTB-4 antagonist |
| Schering-Plough | Proventil (salbutamol) | Bronchodilator/beta-2 agonist |
| | Vanceril (becbomethasone) | Steroid |
| | Mometasone furoate | Steroid |
| | Theo-Dur (theophylline (w/ | |
| | Astra) | |
| | Uni-Dur (theophylline) | |
| | Asmanex (mometasone) | Steroid |
| | CDP 835 (lic from Celitech) | Anti-IL-5 Mab |
| RPR | Intal (disodium cromoglycate) | Anti-inflammatory |
| (Aventis) | Inal/Aarane (disodium | |
| | cromoglycate) | |
| | Tilade (nedocromil sodium) | |
| | Azmacort (triamcinolone | Steroid |
| | acetonide) | |
| | RP 73401 | PDE-4 inhibitor |
| Novartis | Zaditen (ketotifen) | Anti-inflammatory |
| | Azmacort (triamoinolone) | Steroid |
| | Foradil (formoterol) lic from | Bronchodilator/beta-2 agonist |
| | Yamanouchi) | |
| | E25 | Anti-IgE |
| | KCO 912 | K+ Channel opener |
| Merck | Singulair (montelukast) | Leukotriene antagonist Clo- |
| | | Phyllin (theophylline) |
| | Pulinicort Turbuhaler | Steroid |
| | (budesonide) | |
| | Slo-Phyllin (theophylline) | |
| | Symbicort (Pulmicort-Oxis | Steroid |
| | combination) | |
| | Oxis Turbuhaler | Bronchodilator/beta-2 agonist |
| | (D2522/formoterol) | |
| | Roflepanide (Astra) | Steroid |
| | VLA-4 antagoinst (lic from | VLA-4 antagonist |
| | Biogen) | |
| ONO | Onon (pranlukast) | Leukotriene antagonist |
| | Vega (ozagrel) | Thromboxane A2 synthase |
| | | inhibitor |
| Fujisawa | Intal (chromoglycate) | Anti-inflammatory |
| | FK 888 | Neurokine antagonist |
| Forest Labs | Aerobid (flunisolide) | Steroid |
| IVAX | Ventolin (salbutamol) | Bronchodilator/beta-2 agonist |
| | Becotide (beclomethasone | Steroid |
| | Easi-Breathe) | |
| | Serevent (salmeterol) | Bronchodilator/beta-2 agonist |
| | Flixotide (fluticasone) | Steroid |
| | Salbutamol Dry Powder | Bronchodilator/beta-2 agonist |
| | Inhaler | |
| Alza | Volmax (salbutamol) | Bronchodilator/beta-2 agonist |
| Altana | Euphyllin (theophylline) | Xanthine |
| | Ciclesonide | Arachidonic acid antagonist |
| | BY 217 | PDE 4 inhibitor |
| | BY 9010N (ciclesonide) | Steroid (nasal) |
| Tanabe | Flucort (fluocinolone acetonide) | Steroid |
| Seiyaku | | |
| Kissei | Domenan (ozagrel) | Thromboxane A2 synthase |
| | | inhibitor |
| Abbott | Zyflo (zileuton) (4X/day dosing, not competitive w/ Singulair or | |
| | Accolate, no further interest in this area) | |
| Asta Medica | Aerobec (beclomethasone | |
| | dipropionate) (w/ 3M) | |
| | Allergodil (azelastine) | |
| | Allergospasmin (sodium | |
| | cromoglycate reproterol) | |
| | Bronchospasmin (reproterol) | |
| | Salbulair (salbutamol sulphate) | |
| | (w/3M) | |
| | TnNasal (triamcinolone) | Steroid |
| | Fomoterol-MDPI | Beta 2 adrenoceptor agonist |
| | Budesonide-MDPI | |
| UCB | Atenos/Respecal (tulobuerol) | Bronchodilator/beta-2 agonist |
| Recordati | Theodur (theophylline) | Xanthine |
| Medeva | Clickhalers Asmasal, Asmabec (salbutamol beclomethasone | |
| | diproprionate, dry inhaler) | |
| Eisai | E6123 | PAF receptor antagonist |
| Sankyo | Zaditen (ketofen) | Anti-inflammatory |
| | CS 615 | Leukotriene antaonist |
| Shionogi | Anboxan/S 1452 (domitroban) | Thromboxane A2 receptor |
| | | antagonist |
| Yamanouchi | YM 976 | Leukotriene D4/thromboxane |
| | | A2 |
| | | dual antagonist |
| 3M Pharma | Exirel (pirbuterol) | |
| Hoechst | Autoinhalers (3M albuterol | Bronchodilator/beta-2 agonist |
| | projects) | |
| (Aventis) | | |
| SmithKline | Ariflo | PDE-4 inhibitor |
| Beecham | SB 240563 | Anti-IL5 Mab (humanized) |
| | SB 240683 | Anti-IL4 Mab |
| | IDEC 151/clenoliximab | Anti-CD4 Mab, primatised |
| Roche | Anti-IgE(GNE)/CG051901 | Down-regulator of IgE |
| Sepracor | Fomoterol (R, R) | Beta 2 adrenoceptor agonist |
| | Xopenex (levalbuterol) | Beta 2 adrenoceptor agonist |
| Bayer | BAY U 3405 (ramatroban) | Thromboxane A2 antagonist |
| | BAY 16-9996 (once monthly | IL4 mutein |
| | dosing) | |
| | BAY 19-8004 | PDE-4 inhibitor |
| SR Pharma | SRL 172 | Immunomodulation |
| Immunex | Nuance | Soluble IL-4 receptor |
| | | (immunomodulator) |
| Biogen | Anti-VLA-4 | Immunosuppressant |
| Vanguard | VML 530 | Inhibitor of 5-lipox activation |
| | | protein |
| Recordati | Respix (zafurlukast) | Leukotriene antagonist |
| Genetech | Anti-IgE Mab | Down-regulator of IgE |
| Warner | CI-1018 | PDE 4 inhibitor |
| Lambert | | |
| Celltech/ | CDP 835/SCH 55700 (anti- | PDE 4 inhibitor |
| | IL-5) (lic. to Schering-Plough) | |
| Chiroscience | D4418 (w/ Schering-Plough) | PDE 4 inhibitor |
| | CDP 840 (Celltech) | PDE 4 inhibitor |
| AHP | Pda-641 (asthma steroid | |
| | replacement) | |
| Peptide | RAPID Technology Platform | Protease inhibitors |
| Therapeutics | | |
| Coley | CpG DNA | |
| Pharmaceutical | | |
| Group | | |

TABLE 5
Stringency Conditions

| | | | Hybridization | |
|---|---|---|---|---|
| Stringency | Poly-nucleotide | | Temperature and | Wash Temp. |
| Condition | Hybrid | Hybrid Length (bp)¹ | Buffer^{H} | and Buffer^{H} |
| A | DNA:DNA | >50 | 65° C.; 1xSSC -or- | 65° C.; |
| | | | 42° C.; 1xSSC, 50% | 0.3xSSC |
| | | | formamide | |
| B | DNA:DNA | <50 | T_{B}*; 1xSSC | T_{B}*; 1xSSC |
| C | DNA:RNA | >50 | 67° C.; 1xSSC -or- | 67° C.; |
| | | | 45° C.; 1xSSC, 50% | 0.3xSSC |
| | | | formamide | |
| D | DNA:RNA | <50 | T_{D}*; 1xSSC | T_{D}*; 1xSSC |
| E | RNA:RNA | >50 | 70° C.; 1xSSC -or- | 70° C.; |
| | | | 50° C.; 1xSSC, 50% | 0.3xSSC |
| | | | formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% | |
| | | | formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% | |
| | | | formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% | |
| | | | formamide | |
| L | RNA:RNA | <50 | T_{L}*; 2xSSC | T_{L}*; 2xSSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
^{FI}: SSPE (1x SSPE is 0.15M NaCl, 10 mM NaH₂PO₄, and 1.25 mM EDTA, pH 7.4) can be substituted for SSC (1x SSC is 0.15M NaCl and 15 mM sodium citrate) in the hybridization and wash buffers.
T_{B}* − T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6 (log₁₀[Na⁺]) + 0.41(% G + C) − (600/N), where N is the number of bases in the hybrid, and [Na⁺] is the molar concentration of sodium ions in the hybridization buffer ([Na⁺] for 1x SSC = 0.165 M)

TABLE 6

>HG-U133A: 201662_s_at; 152; 617; 2327; Antisense;
TCTGGCATCAGTTTGCTACAGTGAG
>HG-U133A: 201662_s_at; 267; 369; 2376; Antisense;
GAAATGCATGTCTCAAGCTGCAAGG
>HG-U133A: 201662_s_at; 449; 141; 2390; Antisense;
AAGCTGCAAGGCAAACTCCATTCCT
>HG-U133A: 201662_s_at; 34; 135; 2403; Antisense;
AACTCCATTCCTCATATTAAACTAT
>HG-U133A: 201662_s_at; 686; 101; 2429; Antisense;
ACTTCTCATGACGTCACCATTTTTA
>HG-U133A: 201662_s_at; 463; 577; 2437; Antisense;
TGACGTCACCATTTTTAACTGACAG
>HG-U133A: 201662_s_at; 679; 69; 2478; Antisense;
AGACAGCAAACTTGTGTCTGTCTCT
>HG-U133A: 201662_s_at; 229; 679; 2531; Antisense;
TTTACCACCTATGACTGTACTTGTC
>HG-U133A: 201662_s_at; 153; 75; 2588; Antisense;
AGCAGTGATTTTAAAACCTCAAGTT
>HG-U133A: 201662_s_at; 641; 433; 2817; Antisense;
GTTGCTGTGTAATTATTGTCTTGTA
>HG-U133A: 201662_s_at; 437; 163; 2827; Antisense;
AATTATTGTCTTGTATGCATTTGAG
>HG-U133A: 200728_at; 292; 361; 2959; Antisense;
GAACAGATAAGTTTGCCTGCATGCT
>HG-U133A: 200728_at; 495; 199; 2978; Antisense;
CATGCTGGACATGCCTCAGAACCAT
>HG-U133A: 200728_at; 684; 631; 2993; Antisense;
TCAGAACCATGAATAGCCCGTACTA
>HG-U133A: 200728_at; 648; 657; 3006; Antisense;
TAGCCCGTACTAGATCTTGGGAACA
>HG-U133A: 200728_at; 441; 529; 3025; Antisense;
GGAACATGGATCTTAGAGTCACTTT
>HG-U133A: 200728_at; 110; 243; 3090; Antisense;
CGGGGCTTGTTAAAGGACGCGTATG
>HG-U133A: 200728_at; 493; 423; 3110; Antisense;
GTATGTAGGGCCCGTACCTACTGGC
>HG-U133A: 200728_at; 365; 89; 3125; Antisense;
ACCTACTGGCAGTTGGGTTCAGGGA
>HG-U133A: 200728_at; 414; 117; 3149; Antisense;
AAATGGGATTGACTTGGCCTTCAGG
>HG-U133A: 200728_at; 174; 211; 3167; Antisense;
CTTCAGGCTCCTTTGGTCATAATTT
>HG-U133A: 200728_at; 82; 143; 3246; Antisense;
AAGAGCATTTATCGTTTGTCCCTTG
>HG-U133A: 202820_at; 127; 343; 4969; Antisense;
GAATAGCCTGAACCTGGGAATCGGA
>HG-U133A: 202820_at; 58; 187; 5026; Antisense;
CAGCCTGGCAATAGACCGAGCTCCG
>HG-U133A: 202820_at; 363; 41; 5116; Antisense;
ATGGCTTCGGACAAAATATCTCTGA
>HG-U133A: 202820_at; 396; 115; 5129; Antisense;
AAATATCTCTGAGTTCTGTGTATTT
>HG-U133A: 202820_at; 82; 677; 5153; Antisense;
TTCAGTCAAAACTTTAAACCTGTAG
>HG-U133A: 202820_at; 479; 121; 5168; Antisense;
AAACCTGTAGAATCAATTTAAGTGT
>HG-U133A: 202820_at; 253; 639; 5220; Antisense;
TAATTTGTTTCCAGCATGAGGTATC
>HG-U133A: 202820_at; 590; 571; 5225; Antisense;
TGTTTCCAGCATGAGGTATCTAAGG
>HG-U133A: 202820_at; 506; 67; 5254; Antisense;
AGACCAGAGGTCTAGATTAATACTC
>HG-U133A: 202820_at; 389; 689; 5329; Antisense;
TTACTCTCTTCCACATGTTACTGGA
>HG-U133A: 202820_at; 569; 575; 5394; Antisense;
TGATGACAATCAGTTATACAGTTAT
>HG-U133A: 212175_s_at; 363; 213; 1007; Antisense;
CTTTTATCTCAGAACCCCATGGGTT
>HG-U133A: 212175_s_at; 620; 637; 1053; Antisense;
TCAAATTGTTGTCCTGTCTGTCTAT
>HG-U133A: 212175_s_at; 500; 389; 1091; Antisense;
GAGCTTTGATTACTGACTCCGGTTC
>HG-U133A: 212175_s_at; 200; 1; 1261; Antisense;
CCCTGACTTACCACTAATTTACTAG
>HG-U133A: 212175_s_at; 542; 629; 1297; Antisense;
TCATGAGTAACCTCTCACAGCTACC
>HG-U133A: 212175_s_at; 15; 275; 1350; Antisense;
CCTTCTTTTATCTGCACTGTGTGAA
>HG-U133A: 212175_s_at; 511; 339; 1396; Antisense;
GCAAGTGTCCTAAGCTATGTCATCC
>HG-U133A: 212175_s_at; 142; 461; 1414; Antisense;
GTCATCCAAAGATTGTCCTTTCCAT
>HG-U133A: 212175_s_at; 362; 701; 1433; Antisense;
TTCCATTCTCAAATCCTGTGACTGG
>HG-U133A: 212175_s_at; 586; 381; 1452; Antisense;
GACTGGGATCACTCAACAGCACTGT
>HG-U133A: 212175_s_at; 378; 139; 984; Antisense;
AAGCCAGTGCTCTAAGACCTCAGCT
>HG-U133A: 202022_at; 421; 311; 1035; Antisense;
GCTGCCACTGAGGAGTTCATCAAGC
>HG-U133A: 202022_at; 532; 75; 1124; Antisense;
AGCAGCACAGTCACTCTACATTGCC
>HG-U133A: 202022_at; 250; 285; 1147; Antisense;
CCAACCATGCCTACTGAGTATCCAC
>HG-U133A: 202022_at; 320; 653; 1158; Antisense;
TACTGAGTATCCACTCCATACCACA
>HG-U133A: 202022_at; 374; 591; 1201; Antisense;
TGCACCCACTTTTGCTTGTAGTCAT
>HG-U133A: 202022_at; 323; 549; 1226; Antisense;
GGCCAGGGCCAAATAGCTATGCAGA
>HG-U133A: 202022_at; 48; 67; 1248; Antisense;
AGAGCAGAGATGCCTTCACCTGGCA
>HG-U133A: 202022_at; 349; 207; 1311; Antisense;
CATTGCTGCACCTGGGACCATAGGA
>HG-U133A: 202022_at; 649; 519; 1338; Antisense;
GGAGGATAGGGAGCCCCTCATGACT
>HG-U133A: 202022_at; 142; 615; 1427; Antisense;
TCCCACAATTTTCCCATGATGAGGT
>HG-U133A: 202022_at; 643; 233; 994; Antisense;
CTGCACTCAATGCCTGGCGAGGGCA
>HG-U133A: 201051_at; 477; 475; 1202; Antisense;
GTGAGCATTTGTTCCTGACTCTCAA
>HG-U133A: 201051_at; 668; 671; 1236; Antisense;
TTTGGAGTTCTCTTACGTTTCCTGG
>HG-U133A: 201051_at; 51; 547; 1297; Antisense;
GGCTGGTCTCAGTTTGGTTACTCAA
>HG-U133A: 201051_at; 377; 333; 1339; Antisense;
GCACCAGCCATATCTTTTGCTTTGG
>HG-U133A: 201051_at; 397; 637; 1365; Antisense;
TCACATGATGATACCTGCTTTTCTC
>HG-U133A: 201051_at; 687; 179; 1404; Antisense;
CATCCAACGCCCTGGTTTGTAAATA
>HG-U133A: 201051_at; 502; 669; 1446; Antisense;
TTTGGCACTGGTCTGGGGACATTCC
>HG-U133A: 201051_at; 423; 675; 1486; Antisense;
TTTCCCCCTTCACAGATGGTGGTGG
>HG-U133A: 201051_at; 664; 527; 1528; Antisense;
GGACTCTGATGTTACTCTTGAGCTT
>HG-U133A: 201051_at; 208; 363; 1568; Antisense;
GAAAACCGCAGGCTTGTTGTGTTAA
>HG-U133A: 201051_at; 32; 361; 1630; Antisense;
GAAACACACCTTCAAACTTCAACTT
>HG-U133A: 201301_s_at; 679; 367; 1023; Antisense;
GAAAGTCTCTGTACTCGTTCATCAA
>HG-U133A: 201301_s_at; 103; 167; 1045; Antisense;
CAAGGGTGACACATCTGGAGACTAC
>HG-U133A: 201301_s_at; 577; 119; 1073; Antisense;
AAAGTACTGCTTGTTCTCTGTGGAG
>HG-U133A: 201301_s_at; 697; 527; 1125; Antisense;
GGACAGGAGGATTCTCAACACTTTG
>HG-U133A: 201301_s_at; 130;625; 1170; Antisense;
TCTACACTGCTATTATCATTATCTC
>HG-U133A: 201301_s_at; 499; 505; 718; Antisense;
GGTGAAATTTCTAACTGTTCTCTGT
>HG-U133A: 201301_s_at; 197; 571; 733; Antisense;
TGTTCTCTGTTCCCGGAACCGAAAT
>HG-U133A: 201301_s_at; 83; 533; 747; Antisense;
GGAACCGAAATCACCTGTTGCATGT
>HG-U133A: 201301_s_at; 591; 587; 844; Antisense;
TGAAGATGCTCTGCTGGCTATAGTA
>HG-U133A: 201301_s_at; 416; 537; 932; Antisense;
GGCACCGATGATAACACCCTCATCA
>HG-U133A: 201301_s_at; 263; 13; 993; Antisense;
ATATCCGGGCACACTTCAAGAGACT
>HG-U133A: 206177_s_at; 64;3 155; 1006; Antisense;
CAAGCCTATTGACTACCTTAACCCA
>HG-U133A: 206177_s_at; 420; 439; 1113; Antisense;
GTTATCCTTCTAAAGACTTGTTCTT
>HG-U133A: 206177_s_at; 36; 221; 1168; Antisense;
CTCTACAAATTCCCTCTTGGTGTAA
>HG-U133A: 206177_s_at; 70; 645; 1322; Antisense;
TAAGCACACTTACATAAGCCCCCAT
>HG-U133A: 206177_s_at; 339; 83; 1338; Antisense;
AGCCCCCATACATAGAGTGGGACTC
>HG-U133A: 206177_s_at; 352; 483; 1354; Antisense;
GTGGGACTCTTGGAATCAGGAGACA
>HG-U133A: 206177_s_at; 33; 47; 1371; Antisense;
AGGAGACAAAGCTACCACATGTGGA
>HG-U133A: 206177_s_at; 263; 119; 1395; Antisense;
AAAGGTACTATGTGTCCATGTCATT
>HG-U133A: 206177_s_at; 164; 129; 950; Antisense;
AACACAGCAGTTGCAATAACCTTGG
>HG-U133A: 206177_s_at; 316; 21; 965; Antisense;
ATAACCTTGGCTTGTTTCGGACTTG
>HG-U133A: 206177_s_at; 525; 527; 983; Antisense;
GGACTTGCTCGGGAGGGTAATCACA
>HG-U133A: 219094_at; 417; 705; 2294; Antisense;
TTGAATGTTTCCATGTACCTCACTT
>HG-U133A: 219094_at; 699; 275; 2304; Antisense;
CCATGTACCTCACTTTATTTCAGTT
>HG-U133A: 219094_at; 304; 671; 2381; Antisense;
TTTGGTACATCTAAGTTTTCACTTA
>HG-U133A: 219094_at; 566; 517; 2455; Antisense;
GGATGTTGACGCCAATGTTCAGTTT
>HG-U133A: 219094_at; 79; 573; 2470; Antisense;
TGTTCAGTTTGGGTACGTTGGTGTA
>HG-U133A: 219094_at; 282; 505; 2481; Antisense;
GGTACGTTGGTGTATTGCAAGGGGA
>HG-U133A: 219094_at; 543; 509; 2587; Antisense;
GGTTATTAGGGCCCATTAGAAACAG
>HG-U133A: 219094_at; 352; 141; 2635; Antisense;
AAGCTCTAAAAAACCATCTCATGGA
>HG-U133A: 219094_at; 315; 23; 2771; Antisense;
ATCATGTTCTAGAAATACCTGCAAC
>HG-U133A: 219094_at; 341; 17; 2785; Antisense;
ATACCTGCAACATGACAGTCTAATC
>HG-U133A: 219094_at; 708; 37; 2837; Antisense;
ATGTGTATAATTTCCTGGTAAGGCT
>HG-U133A: 206743_s_at; 624; 217; 1000; Antisense;
CTACCGCTGGGTCTGCGAGACAGAG
>HG-U133A: 206743_s_at; 600; 497; 1008; Antisense;
GGGTCTGCGAGACAGAGCTGGACAA
>HG-U133A: 206743_s_at; 555; 597; 1084; Antisense;
TGCCGCAGGGGTCCGGGATTGGGAA
>HG-U133A: 206743_s_at; 70; 625; 1126; Antisense;
TCTTCTGCTTTCTCGGGAATTTTCA
>HG-U133A: 206743_s_at; 609; 223; 1137; Antisense;
CTCGGGAATTTTCATCTAGGATTTT
>HG-U133A: 206743_s_at; 292; 417; 1176; Antisense;
GATAGGGTGATGTTCCGAAGGTGAG
>HG-U133A: 206743_s_at; 290; 501; 1195; Antisense;
GGTGAGGAGCTTGAAACCCGTGGCG
>HG-U133A: 206743_s_at; 157; 47; 771; Antisense;
AGGAGCAGAAATTTGTCCAGCACCA
>HG-U133A: 206743_s_at; 181; 369; 778; Antisense;
GAAATTTGTCCAGCACCACATAGGC
>HG-U133A: 206743_s_at; 487; 283; 793; Antisense;
CCACATAGGCCCTGTGAACACCTGG
>HG-U133A: 206743_s_at; 328; 389; 899; Antisense;
GAGCAGCCGGACGACTGGTACGGCC
>HG-U133A: 212297_at; 420; 435; 1883; Antisense;
GTTCCCCATGTTTATGAAAGTCCTG
>HG-U133A: 212297_at; 329; 113; 2014; Antisense;
AAATATTCATGCATGCAATTTTGAC
>HG-U133A: 212297_at; 23; 565; 2058; Antisense;
TGTATATTTATGGTGGGAGGTGGTT
>HG-U133A: 212297_at; 25; 163; 2109; Antisense;
AATTTTTGTACAGTCTGTGGGCATT
>HG-U133A: 212297_at; 127; 465; 2121; Antisense;
GTCTGTGGGCATTTACACATTTTTA
>HG-U133A: 212297_at; 391; 147; 2188; Antisense;
AAGTTACTTCTAGTTATGATTTGTG
>HG-U133A: 212297_at; 517; 423; 2205; Antisense;
GATTTGTGAATTCCCTAAGACCTTG
>HG-U133A: 212297_at; 619; 155; 2257; Antisense;
AATGATACTGCATCTTTATATTTTT
>HG-U133A: 212297_at; 516; 113; 2283; Antisense;
AAATTGTATTGCTGCTCAAGAATGG
>HG-U133A: 212297_at; 38; 19; 2301; Antisense;
AGAATGGTACCCTCTTGTCAAAAAG
>HG-U133A: 212297_at; 199; 205; 2331; Antisense;
CATTCATAATTGTACATTCAGCATT
>HG-U133A: 202391_at; 154; 19; 1003; Antisense;
ATACCTTCAGTCAACTTTACCAAGA
>HG-U133A: 202391_at; 521; 467; 1029; Antisense;
GTCCTGGATTTCCAAGATCCGCGTC
>HG-U133A: 202391_at; 661; 97; 1099; Antisense;
ACTCCTCCACCGCTGAGAGTTGAAT
>HG-U133A: 202391_at; 440; 17; 1122; Antisense;
ATAGCTTTTCTTCTGCAATGGGAGT
>HG-U133A: 202391_at; 353; 521; 1149; Antisense;
GGAGTGATGCGTTTGATTCTGCCCA
>HG-U133A: 202391_at; 292; 189; 1293; Antisense;
CAGACAGAGCCCACTTAGCTTGTCC
>HG-U133A: 202391_at; 112; 559; 1321; Antisense;
TGGATCTCAATGCCAATCCTCCATT
>HG-U133A: 202391_at; 617; 205; 1342; Antisense;
CATTCTTCCTCTCCAGATATTTTTG
>HG-U133A: 202391_at; 152; 55; 1369; Antisense;
AGTGACAAACATTCTCTCATCCTAC
>HG-U133A: 202391_at; 141; 657; 1395; Antisense;
TAGCCTACCTAGATTTCTCATGACG
>HG-U133A: 202391_at; 26; 401; 1419; Antisense;
GAGTTAATGCATGTCCGTGGTTGGG
>HG-U133A: 203771_s_at; 97; 189; 267; Antisense;
CAGAGCCCGAGAGGAAGTTTGGCGT
>HG-U133A: 203771_s_at; 399; 59; 276; Antisense;
AGAGGAAGTTTGGCGTGGTGGTGGT
>HG-U133A: 203771_s_at; 255; 265; 315; Antisense;
CCGGCTCCGTGCGGATGAGGGACTT
>HG-U133A: 203771_s_at; 104; 517; 327; Antisense;
GGATGAGGGACTTGCGGAATCCACA
>HG-U133A: 203771_s_at; 378; 233; 368; Antisense;
CTGAACCTGATTGGCTTCGTGTCGA
>HG-U133A: 203771_s_at; 272; 539; 380; Antisense;
GGCTTCGTGTCGAGAAGGGAGCTCG
>HG-U133A: 203771_s_at; 141; 525; 397; Antisense;
GGAGCTCGGGAGCATTGATGGAGTC
>HG-U133A: 203771_s_at; 107; 323; 408; Antisense;
GCATTGATGGAGTCCAGCAGATTTC
>HG-U133A: 203771_s_at; 607; 425; 427; Antisense;
GATTTCTTTGGAGGATGCTCTTTCC
>HG-U133A: 203771_s_at; 112; 417; 440; Antisense;
GATGCTCTTTCCAGCCAAGAGGTGG
>HG-U133A: 203771_s_at; 403; 15; 474; Antisense;
ATATCTGCAGTGAGAGCTCCAGCCA
>HG-U133A: 203082_at; 5; 175; 3608; Antisense;
CAAAGGACAGGCGGAGACCGGCCGT
>HG-U133A: 203082_at; 276; 263; 3629; Antisense;
CCGTCATACGCGAGCCTCATGAAAG
>HG-U133A: 203082_at; 133; 145; 3655; Antisense;
AAGATCCTTGCACTGCTGGATGCTC
>HG-U133A: 203082_at; 394; 595; 3668; Antisense;
TGCTGGATGCTCTGAGTACGGTGCA
>HG-U133A: 203082_at; 339; 355; 3807; Antisense;
GAAGCTCTTCAGAATTCAGGGGCAG
>HG-U133A: 203082_at; 298; 481; 3873; Antisense;
GGGCCAATTGCAGTGAGCCTTTGGA
>HG-U133A: 203082_at; 228; 569; 3908; Antisense;
TGTCCCTGGATCTGCGGAGGTAGAC
>HG-U133A: 203082_at; 418; 345; 3953; Antisense;
GAATGCCTGTGAATGACACGTCAGT
>HG-U133A: 203082_at; 506; 61; 3993; Antisense;
AGATGTCTCTACTCAAACTGTGCCT
>HG-U133A: 203082_at; 209; 239; 4048; Antisense;
CTGGGACTGGGTTCATTCTCATGAC
>HG-U133A: 203082_at; 681; 5; 4062; Antisense;
ATTCTCATGACTTGGGGCTGTCGAG
>HG-U133A: 205548_s_at; 102; 15; 1115; Antisense;
ATATATTGTGCATCAACTCTGTTGG
>HG-U133A: 205548_s_at; 440; 485; 1159; Antisense;
GTGGACGATTTGTTCTAGCACCTTT
>HG-U133A: 205548_s_at; 673; 153; 701; Antisense;
AATGGCCATCAGAATCACTATCCTC
>HG-U133A: 205548_s_at; 42; 607; 724; Antisense;
TCCTCCTGTTCCATTTGGTTATCCA
>HG-U133A: 205548_s_at; 12; 113; 763; Antisense;
AAATAAACCATATCGCCCAATTCCA
>HG-U133A: 205548_s_at; 258; 165; 781; Antisense;
AATTCCAGTGACATGGGTACCTCCT
>HG-U133A: 205548_s_at; 521; 607; 802; Antisense;
TCCTCCTGGAATGCATTGTGACCGG
>HG-U133A: 205548_s_at; 321; 385; 821; Antisense;
GACCGGAATCACTGGATTAATCCTC
>HG-U133A: 205548_s_at; 525; 691; 837; Antisense;
TTAATCCTCACATGTTAGCACCTCA
>HG-U133A: 205548_s_at; 14; 299; 854; Antisense;
GCACCTCACTAACTTCGTTTTTGAT
>HG-U133A: 205548_s_at; 199; 565; 967; Antisense;
TGGGCCAAACCATCAAACTTATTTT
>HG-U133A: 203429_s_at; 613; 399; 5339; Antisense;
GAGATTATTATTCCTTGATGTTTGC
>HG-U133A: 203429_s_at; 30; 579; 5354; Antisense;
TGATGTTTGCTTTGTATTGGCTACA
>HG-U133A: 203429_s_at; 151; 39; 5399; Antisense;
ATGTGATGTCGATGTCTCTGTCTTT
>HG-U133A: 203429_s_at; 177; 393; 5534; Antisense;
GAGAATTGACCATTTATTGTTGTGA
>HG-U133A: 203429_s_at; 456; 571; 5640; Antisense;
TGTAATGTGACTTATTTAACGCCTT
>HG-U133A: 203429_s_at; 384; 699; 5725; Antisense;
TTCCTGTCTGCACAATTAGCTATTC
>HG-U133A: 203429_s_at; 150; 651; 5741; Antisense;
TAGCTATTCAGAGCAAGAGGGCCTG
>HG-U133A: 203429_s_at; 531; 291; 5761; Antisense;
GCCTGATTTTATAGAAGCCCCTTGA
>HG-U133A: 203429_s_at; 450; 355; 5774; Antisense;
GAAGCCCCTTGAAAAGAGGTCCAGA
>HG-U133A: 203429_s_at; 342; 163; 5821; Antisense;
AATTATGTGATCTGTGTGTTGTGGG
>HG-U133A: 203429_s_at; 684; 653; 5868; Antisense;
TACGGAGCTGTAGTGCCATTAGAAA
>HG-U133A: 210054_at; 623; 7; 1891; Antisense;
ATTCTACTCATAGGCTTTACCAAGT
>HG-U133A: 210054_at; 303; 169; 2010; Antisense;
CAAGATCAGTTGGCAGTATCTGCTC
>HG-U133A: 210054_at; 439; 539; 2021; Antisense;
GGCAGTATCTGCTCAAGAACATTCT
>HG-U133A: 210054_at; 604; 673; 2045; Antisense;
TTTCTTTCTGTCCAAACGGAATAAG
>HG-U133A: 210054_at; 659; 485; 2073; Antisense;
GTGGACATGCTTTGTGATACTTTGT
>HG-U133A: 210054_at; 205; 187; 2112; Antisense;
CAGCTTTTGCTTAGTGATCAGGAGT
>HG-U133A: 210054_at; 509; 343; 2171; Antisense;
GAATAAGCTAAATCATCTCCTCACT
>HG-U133A: 210054_at; 348; 203; 2184; Antisense;
CATCTCCTCACTGATATTCTTGCTG
>HG-U133A: 210054_at; 167; 427; 2339; Antisense;
GATTAAGGCTGTTAGTCTTGAAGAT
>HG-U133A: 210054_at; 336; 343; 2382; Antisense;
GAATCTTTATTACGTGTCCTCTTTT
>HG-U133A: 210054_at; 95; 687; 2391; Antisense;
TTACGTGTCCTCTTTTATTTATTAG
>HG-U133A: 222309_at; 410; 41; 141; Antisense;
AGGCTGAAGTAACCTTATTCCTATT
>HG-U133A: 222309_at; 484; 87; 152; Antisense;
ACCTTATTCCTATTGTTTAGTAGCT
>HG-U133A: 222309_at; 411; 57; 170; Antisense;
AGTAGCTAATAGCATGCTTTTGATA
>HG-U133A: 222309_at; 472; 325; 181; Antisense;
GCATGCTTTTGATATGCTTATGATC
>HG-U133A: 222309_at; 684; 3; 270; Antisense;
ATTGTGATGCTGTATCATATTTTAT
>HG-U133A: 222309_at; 254; 1; 297; Antisense;
TACGGTTTATAAGAAAAGCTCCTAG
>HG-U133A: 222309_at; 705; 65; 308; Antisense;
AGAAAAGCTCCTAGGTATAAAATGC
>HG-U133A: 222309_at; 67; 153; 328; Antisense;
AATGCTACATAGCAGGAACTTGGTT
>HG-U133A: 222309_at; 343; 245; 339; Antisense;
GCAGGAACTTGGTTTTTCAATGTTA
>HG-U133A: 222309_at; 56; 37; 358; Antisense;
ATGTTATTATTTCCTACTGTTTTTG
>HG-U133A: 222309_at; 197; 605; 369; Antisense;
TCCTACTGTTTTTGACGTAACGGCA
>HG-U133A: 210244_at; 589; 355; 136; Antisense;
GAAGCTGTGCTTCGTGCTATAGATG
>HG-U133A: 210244_at; 448; 17; 154; Antisense;
ATAGATGGCATCAACCAGCGGTCCT
>HG-U133A: 210244_at; 291; 383; 211; Antisense;
GACCCCAGGCCCACGATGGATGGGG
>HG-U133A: 210244_at; 200; 41; 226; Antisense;
ATGGATGGGGACCCAGACACGCCAA
>HG-U133A: 210244_at; 259; 373; 241; Antisense;
GACACGCCAAAGCCTGTGAGCTTCA
>HG-U133A: 210244_at; 262; 383; 291; Antisense;
GACGACACAGCAGTCACCAGAGGAT
>HG-U133A: 210244_at; 286; 501; 347; Antisense;
GGTGTATGGGGACAGTGACCCTCAA
>HG-U133A: 210244_at; 488; 249; 35; Antisense;
CCCAAAGGGATGGCCACTCCCTGGG
>HG-U133A: 210244_at; 425; 543; 382; Antisense;
GGCTCCTTTGACATCAGTTGTGATA
>HG-U133A: 210244_at; 111; 427; 419; Antisense;
GATTTGCCCTGCTGGGTGATTTCTT
>HG-U133A: 210244_at; 245; 681; 506; Antisense;
TTTTGCGGAATCTTGTACCCAGGAC
>HG-U133A: 207483_s_at; 661; 81; 3884; Antisense;
AGCGATCTGCCATGAGAGCAGTAGC
>HG-U133A: 207483_s_at; 52; 403; 3939; Antisense;
GAGTCCACTGATGAGTGAATTCCAG
>HG-U133A: 207483_s_at; 95; 341; 3955; Antisense;
GAATTCCAGTCACAGATCAGTTCTA
>HG-U133A: 207483_s_at; 58; 421; 3969; Antisense;
GATCAGTTCTAACCCTGAGCTGGCG
>HG-U133A: 207483_s_at; 233; 389; 3985; Antisense;
GAGCTGGCGGCTATCTTTGAAAGTA
>HG-U133A: 207483_s_at; 482; 629; 4021; Antisense;
TCATCATCTACTAACTTGGAATCAA
>HG-U133A: 207483_s_at; 287; 371; 4048; Antisense;
GACACTAGTTAGATGTTTGTTCACC
>HG-U133A: 207483_s_at; 621; 443; 4062; Antisense;
GTTTGTTCACCATGGGGACCATTAC
>HG-U133A: 207483_s_at; 40; 501; 4075; Antisense;
GGGGACCATTACATATGACCATACA
>HG-U133A: 207483_s_at; 482; 13; 4247; Antisense;
ATTTCCATAATCCAGAGGTTGTAAA
>HG-U133A: 207483_s_at; 237; 505; 4349; Antisense;
GGTCCAGTATCTATTTACCCTGTAA
>HG-U133A: 211922_s_at; 290; 541; 1036; Antisense;
GGCATTGAGGCCAGTCCTGACAAAA
>HG-U133A: 211922_s_at; 345; 249; 1105; Antisense;
CGCCTGGGACCCAATTATCTTCATA
>HG-U133A: 211922_s_at; 626; 627; 1125; Antisense;
TCATATACCTGTGAACTGTCCCTAC
>HG-U133A: 211922_s_at; 190; 549; 1180; Antisense;
GGCCCGATGTGCATGCAGGACAATC
>HG-U133A: 211922_s_at; 14; 319; 1235; Antisense;
GCTTTGGTGCTCCGGAACAACAGCC
>HG-U133A: 211922_s_at; 336; 471; 1297; Antisense;
GTGCGGAGATTCAACACTGCCAATG
>HG-U133A: 211922_s_at; 541; 645; 1326; Antisense;
TAACGTTACTCAGGTGCGGGCATTC
>HG-U133A: 211922_s_at; 459; 639; 1448; Antisense;
TCAAGAACTTCACTGAGGTCCACCC
>HG-U133A: 211922_s_at; 269; 583; 1473; Antisense;
TGACTACGGGAGCCACATCCAGGCT
>HG-U133A: 211922_s_at; 480; 143; 1528; Antisense;
AAGAATGCGATTCACACCTTTGTGC
>HG-U133A: 211922_s_at; 665; 509; 996; Antisense;
GGTTGAACAGATAGCCTTCGACCCA
>HG-U133A: 221427_s_at; 408; 143; 583; Antisense;
AAGAAAGCCAAGGCGGACAGCCCCG
>HG-U133A: 221427_s_at; 416; 243; 649; Antisense;
CGGAGCCGTGAGCAGAGCTACTCGA
>HG-U133A: 221427_s_at; 202; 611; 682; Antisense;
TCCCGATCAGCGTCTCCTAAGAGGA
>HG-U133A: 221427_s_at; 660; 361; 708; Antisense;
GAAAAGTGACAGCGGCTCCACATCT
>HG-U133A: 221427_s_at; 20; 179; 726; Antisense;
CACATCTGGTGGGTCCAAGTCGCAG
>HG-U133A: 221427_s_at; 332; 83; 751; Antisense;
AGCCGCTCCCGGAGCAGGAGTGACT
>HG-U133A: 221427_s_at; 208; 249; 801; Antisense;
CGCTCCCTACAAAGGCTCTGAGATT
>HG-U133A: 221427_s_at; 488; 573; 819; Antisense;
TGAGATTCGGGGCTCCCGGAAGTCC
>HG-U133A: 221427_s_at; 28; 169; 873; Antisense;
CAAGTCTCGGAGCCGGAGTTCTTCC
>HG-U133A: 221427_s_at; 546; 659; 894; Antisense;
TTCCCGTTCTCGAAGCAGGTCACGG
>HG-U133A: 221427_s_at; 2; 409; 990; Antisense;
GAGGTCGTATGAACGCACAGGCCGT
>HG-U133A: 213743_at; 545; 707; 272; Antisense;
TTGTGTGAGCTATTCAAACTCTTCA
>HG-U133A: 213743_at; 15; 101; 289; Antisense;
ACTCTTCAACCCCTGAACAGGGTAT
>HG-U133A: 213743_at; 442; 361; 303; Antisense;
GAACAGGGTATTAAGCTTCCAAAAT
>HG-U133A: 213743_at; 39; 123; 381; Antisense;
AAACCCTTATAATTCATACTATCAT
>HG-U133A: 213743_at; 633; 341; 406; Antisense;
GAATTTGCTTTATCCATCTCATTTG
>HG-U133A: 213743_at; 477; 25; 421; Antisense;
ATCTCATTTGCATAACAGTTCATCT
>HG-U133A: 213743_at; 623; 645; 433; Antisense;
TAACAGTTCATCTGTCTGGTCCCAT
>HG-U133A: 213743_at; 12; 487; 450; Antisense;
GGTCCCATTAGGCTCTACCAAAGAA
>HG-U133A: 213743_at; 58; 579; 484; Antisense;
TGAGTGGACATTATTACTGTGACTC
>HG-U133A: 213743_at; 87; 97; 499; Antisense;
ACTGTGACTCTTGTAAGTAGCCATA
>HG-U133A: 213743_at; 379; 47; 549; Antisense;
AGGTATGAAATTCCACATGTGCAAA
>HG-U133A: 206978_at; 39; 605; 1661; Antisense;
TCCATCGCTGTCATCTCAGCTGGAT
>HG-U133A: 206978_at; 417; 697; 1691; Antisense;
TTCTCTCAGGCTTGCTGCCAAAAGC
>HG-U133A: 206978_at; 670; 1; 1763; Antisense;
ATTCGAGTGTTTCAGTGCTTCGCAG
>HG-U133A: 206978_at; 540; 471; 1777; Antisense;
GTGCTTCGCAGATGTCCTTGATGCT
>HG-U133A: 206978_at; 580; 313; 1799; Antisense;
GCTCATATTGTTCCCTAATTTGCCA
>HG-U133A: 206978_at; 289; 101; 1919; Antisense;
ACTTTCCTCTTAGTCGAGCCAAGTT
>HG-U133A: 206978_at; 442; 481; 1966; Antisense;
GTGTGTTTCTGATCTGATGCAAGCA
>HG-U133A: 206978_at; 43; 563; 1999; Antisense;
TGGGCTTCTAGAACCAGGCAACTTG
>HG-U133A: 206978_at; 33; 529; 2024; Antisense;
GGAACTAGACTCCCAAGCTGGACTA
>HG-U133A: 206978_at; 682; 305; 2040; Antisense;
GCTGGACTATGGCTCTACTTTCAGG
>HG-U133A: 206978_at; 463; 373; 2100; Antisense;
GACAGAGCAGAACTTTCACCTTCAT
>HG-U133A: 201743_at; 660; 469; 1002; Antisense;
GTGCCTAAAGGACTGCCAGCCAAGC
>HG-U133A: 201743_at; 608; 287; 1020; Antisense;
GCCAAGCTCAGAGTGCTCGATCTCA
>HG-U133A: 201743_at; 590; 337; 1048; Antisense;
GCAACAGACTGAACAGGGCGCCGCA
>HG-U133A: 201743_at; 283; 581; 1076; Antisense;
TGACGAGCTGCCCGAGGTGGATAAC
>HG-U133A: 201743_at; 571; 233; 1101; Antisense;
CTGACACTGGACGGGAATCCCTTCC
>HG-U133A: 201743_at; 136; 95; 1150; Antisense;
ACGAGGGCTCAATGAACTCCGGCGT
>HG-U133A: 201743_at; 284; 257; 1243; Antisense;
CCCGGGGCTTTGCCTAAGATCCAAG
>HG-U133A: 201743_at; 620; 489; 1306; Antisense;
GGGAGTCCCGTCAGGACGTTGAGGA
>HG-U133A: 201743_at; 53; 579; 1325; Antisense;
TGAGGACTTTTCGACCAATTCAACC
>HG-U133A: 201743_at; 565; 277; 799; Antisense;
CCATCCAGAATCTAGCGCTGCGCAA
>HG-U133A: 201743_at; 392; 255; 929; Antisense;
CCCTAGCGCTCCGAGATGCATGTGG
>HG-U133A: 203645_s_at; 544; 285; 3126; Antisense;
GCCAGACGCTGGGGCCATAGTGAGT
>HG-U133A: 203645_s_at; 295; 245; 3237; Antisense;
CGTCAGTCATCCTTTATTGCAGTCG
>HG-U133A: 203645_s_at; 351; 667; 3251; Antisense;
TATTGCAGTCGGGATCCTTGGGGTT
>HG-U133A: 203645_s_at; 121; 551; 3284; Antisense;
GGCCATTTTCGTCGCATTATTCTTC
>HG-U133A: 203645_s_at; 320; 191; 3327; Antisense;
CAGAGACAGCGGCTTGCAGTTTCCT
>HG-U133A: 203645_s_at; 202; 687; 3366; Antisense;
TTAGTCCACCAAATTCAATACCGGG
>HG-U133A: 203645_s_at; 464; 655; 3384; Antisense;
TACCGGGAGATGAATTCTTGCCTGA
>HG-U133A: 203645_s_at; 418; 293; 3445; Antisense;
GCCATTCTGAGCCACACTGAAAAGG
>HG-U133A: 203645_s_at; 493; 19; 3483; Antisense;
ATAACCCAGTGAGTTCAGCCTTTAA
>HG-U133A: 203645_s_at; 209; 557; 3538; Antisense;
TGGAGCAGAAATTCACCTCTCTCAC
>HG-U133A: 203645_s_at; 66; 511; 3587; Antisense;
GGAGTTCTTCTTCTCCTAGGATTCC
>HG-U133A: 215049_x_at; 296; 245; 3237; Antisense;
CGTCAGTCATCCTTTATTGCAGTCG
>HG-U133A: 215049_x_at; 350; 667; 3251; Antisense;
TATTGCAGTCGGGATCCTTGGGGTT
>HG-U133A: 215049_x_at; 122; 551; 3284; Antisense;
GGCCATTTTCGTCGCATTATTCTTC
>HG-U133A: 215049_x_at; 321; 191; 3327; Antisense;
CAGAGACAGCGGCTTGCAGTTTCCT
>HG-U133A: 215049_x_at; 482; 163; 3444; Antisense;
AATTCCCATGAGTCAGCTGATTTCA
>HG-U133A: 215049_x_at; 165; 119; 3521; Antisense;
AAAGGAGGCCATTCTGAGCCACACT
>HG-U133A: 215049_x_at; 494; 19; 3566; Antisense;
ATAACCCAGTGAGTTCAGCCTTTAA
>HG-U133A: 215049_x_at; 210; 557; 3621; Antisense;
TGGAGCAGAAATTCACCTCTCTCAC
>HG-U133A: 215049_x_at; 607; 635; 3633; Antisense;
TCACCTCTCTCACTGACTATTACAG
>HG-U133A: 215049_x_at; 67; 511; 3670; Antisense;
GGAGTTCTTCTTCTCCTAGGATTCC
>HG-U133A: 215049_x_at; 628; 215; 3685; Antisense;
CTAGGATTCCTAAGACTGCTGCTGA
>HG-U133A: 208651_x_at; 279; 239; 1406; Antisense;
CTGGGATTACAGGCTTGAGCCCCCG
>HG-U133A: 208651_x_at; 239; 303; 1430; Antisense;
GCGCCCAGCCATCAAAATGCTTTTT
>HG-U133A: 208651_x_at; 53; 319; 1448; Antisense;
GCTTTTTATTTCTGCATATGTTTGA
>HG-U133A: 208651_x_at; 247; 637; 1612; Antisense;
TCACAAACTTTTATACTCTTTCTGT
>HG-U133A: 208651_x_at; 482; 617; 1632; Antisense;
TCTGTATATACATTTTTTTTCTTTA
>HG-U133A: 208651_x_at; 168; 161; 1676; Antisense;
AATAGCCACATTTAGAACACTTTTT
>HG-U133A: 208651_x_at; 128; 131; 1691; Antisense;
AACACTTTTTGTTATCAGTCAATAT
>HG-U133A: 208651_x_at; 572; 415; 1721; Antisense;
GATAGTTAGAACCTGGTCCTAAGCC
>HG-U133A: 208651_x_at; 309; 357; 1729; Antisense;
GAACCTGGTCCTAAGCCTAAAAGTG
>HG-U133A: 208651_x_at; 308; 149; 1749; Antisense;
AAGTGGGCTTGATTCTGCAGTAAAT
>HG-U133A: 208651_x_at; 229; 649; 1769; Antisense;
TAAATCTTTTACAACTGCCTCGACA
>HG-U133A: 209771_x_at; 248; 637; 1931; Antisense;
TCACAAACTTTTATACTCTTTCTGT
>HG-U133A: 209771_x_at; 310; 357; 2048; Antisense;
GAACCTGGTCCTAAGCCTAAAAGTG
>HG-U133A: 209771_x_at; 309; 149; 2068; Antisense;
AAGTGGGCTTGATTCTGCAGTAAAT
>HG-U133A: 209771_x_at; 230; 649; 2088; Antisense;
TAAATCTTTTACAACTGCCTCGACA
>HG-U133A: 209771_x_at; 549; 291; 2104; Antisense;
GCCTCGACACACATAAACCTTTTTA
>HG-U133A: 209771_x_at; 390; 159; 2131; Antisense;
AATAGACACTCCCCGAAGTCTTTTG
>HG-U133A: 209771_x_at; 234; 147; 2146; Antisense;
AAGTCTTTTGTTCGCATGGTCACAC
>HG-U133A: 209771_x_at; 647; 663; 2201; Antisense;
TATGGCCACAGTAGTCTTGATGACC
>HG-U133A: 209771_x_at; 421; 581; 2221; Antisense;
TGACCAAAGTCCTTTTTTTCCATCT
>HG-U133A: 209771_x_at; 707; 361; 2306; Antisense;
GAACACTCTTGCTTTATTCCAGAAT
>HG-U133A: 209771_x_at; 240; 479; 2365; Antisense;
GTGTATTTACGCTTTGATTCATAGT
>HG-U133A: 216379_x_at; 246; 637; 1447; Antisense;
TCACAAACTTTTATACTCTTTCTGT
>HG-U133A: 216379_x_at; 308; 357; 1564; Antisense;
GAACCTGGTCCTAAGCCTAAAAGTG
>HG-U133A: 216379_x_at; 307; 149; 1584; Antisense;
AAGTGGGCTTGATTCTGCAGTAAAT
>HG-U133A: 216379_x_at; 228; 649; 1604; Antisense;
TAAATCTTTTACAACTGCCTCGACA
>HG-U133A: 216379_x_at; 548; 291; 1620; Antisense;
GCCTCGACACACATAAACCTTTTTA
>HG-U133A: 216379_x_at; 389; 159; 1647; Antisense;
AATAGACACTCCCCGAAGTCTTTTG
>HG-U133A: 216379_x_at; 114; 351; 1661; Antisense;
GAAGTCTTTTGTTCGCATGGTCACA
>HG-U133A: 216379_x_at; 398; 199; 1676; Antisense;
CATGGTCACACACTGATGCTTAGAT
>HG-U133A: 216379_x_at; 646; 663; 1716; Antisense;
TATGGCCACAGTAGTCTTGATGACC
>HG-U133A: 216379_x_at; 420; 581; 1736; Antisense;
TGACCAAAGTCCTTTTTTTCCATCT
>HG-U133A: 216379_x_at; 706L 361; 1821; Antisense;
GAACACTCTTGCTTTATTCCAGAAT
>HG-U133A: 209795_at; 186; 657; 1143; Antisense;
TAGTCTAATTGAATCCCTTAAACTC
>HG-U133A: 209795_at; 111; 39; 1273; Antisense;
ATGGGATGATCGTGTATTTATTTTT
>HG-U133A: 209795_at; 610; 679; 1294; Antisense;
TTTTTTACTTCCTCAGCTGTAGACA
>HG-U133A: 209795_at; 175; 103; 1300; Antisense;
ACTTCCTCAGCTGTAGACAGGTCCT
>HG-U133A: 209795_at; 332; 375; 1315; Antisense;
GACAGGTCCTTTTCGATGGTACATA
>HG-U133A: 209795_at; 418; 559; 1331; Antisense;
TGGTACATATTTCTTTGCCTTTATA
>HG-U133A: 209795_at; 577; 667; 1352; Antisense;
TATAATCTTTTATACAGTGTCTTAC
>HG-U133A: 209795_at; 109; 477; 1450; Antisense;
GTGATGTGGCAAATCTCTATTAGGA
>HG-U133A: 209795_at; 347; 13; 1476; Antisense;
ATATTCTGTAATCTTCAGACCTAGA
>HG-U133A: 209795_at; 244; 39; 1520; Antisense;
AGGTTTGTGACTTTCCTAAATCAAT
>HG-U133A: 209795_at; 75; 655; 1550; Antisense;
TACGTGCAATACTTCAATACTTCAT
>HG-U133A: 204440_at; 206; 605; 1726; Antisense;
TCCATTTCTCATGTTTTCCATTGTT
>HG-U133A: 204440_at; 127; 169; 1769; Antisense;
CAAGAAGCCTTTCCTGTAGCCTTCT
>HG-U133A: 204440_at; 167; 469; 1827; Antisense;
GTCCACGGTCTGTTCTTGAAGCAGT
>HG-U133A: 204440_at; 613; 587; 1843; Antisense;
TGAAGCAGTAGCCTAACACACTCCA
>HG-U133A: 204440_at; 654; 143; 1867; Antisense;
AAGATATGGACACACGGGAGCCGCT
>HG-U133A: 204440_at; 256; 441; 1925; Antisense;
GTTTTAGCCATTGTTGGCTTTCCCT
>HG-U133A: 204440_at; 83; 541; 1939; Antisense;
TGGCTTTCCCTTATCAAACTTGGGC
>HG-U133A: 204440_at; 558; 235; 1997; Antisense;
CTGAGTTATATGTTCACTGTCCCCC
>HG-U133A: 204440_at; 288; 437; 2008; Antisense;
GTTCACTGTCCCCCTAATATTAGGG
>HG-U133A: 204440_at; 344; 353; 2216; Antisense;
GAACCCCCATGATGTAAGTTTACCT
>HG-U133A: 204440_at; 227; 123; 2248; Antisense;
AAACCTGCACTTATACCCATGAACT
>HG-U133A: 205627_at; 658; 567; 322; Antisense;
TGTGCTGAACGGACCGCTATCCAGA
>HG-U133A: 205627_at; 484; 215; 338; Antisense;
CTATCCAGAAGGCCGTCTCAGAAGG
>HG-U133A: 205627_at; 167; 341; 379; Antisense;
GCAATTGCTATCGCCAGTGACATGC
>HG-U133A: 205627_at; 32; 503; 427; Antisense;
GGGGCCTGCAGGCAAGTCATGAGAG
>HG-U133A: 205627_at; 228; 395; 447; Antisense;
GAGAGAGTTTGGCACCAACTGGCCC
>HG-U133A: 205627_at; 689; 295; 468; Antisense;
GCCCGTGTACATGACCAAGCCGGAT
>HG-U133A: 205627_at; 700; 569; 504; Antisense;
TGTCATGACGGTCCAGGAGCTGCTG
>HG-U133A: 205627_at; 556; 603; 535; Antisense;
TCCTTTGGGCCTGAGGACCTGCAGA
>HG-U133A: 205627_at; 88; 55; 566; Antisense;
AGTGACAGCCAGAGAATGCCCACTG
>HG-U133A: 205627_at; 643; 597; 735; Antisense;
TGCCTTGGGACTTAGAACACCGCCG
>HG-U133A: 205627_at; 308; 469; 807; Antisense;
GTCCAGCCTAGTCTGGACTGCTTCC
>HG-U133A: 206676_at; 114; 7; 1759; Antisense;
ATTGCCAATTCTTTAAGTGTTTTCT
>HG-U133A: 206676_at; 559; 687; 1812; Antisense;
TTAAGCTATCTATACCTTACTGCAA
>HG-U133A: 206676_at; 224; 273; 1888; Antisense;
CCTACCTGACTGCCACAGAACTGGG
>HG-U133A: 206676_at; 592; 59; 1963; Antisense;
AGTTCAGTGAGAATCTGCTGTCTTT
>HG-U133A: 206676_at; 636; 53; 2081; Antisense;
AGTGTCTAATCTATCGTGTCAACCC
>HG-U133A: 206676_at; 143; 15; 2089; Antisense;
ATCTATCGTGTCAACCCCAAATTTT
>HG-U133A: 206676_at; 99; 653; 2114; Antisense;
TACGTATGAGATCCTTTAGTCCACC
>HG-U133A: 206676_at; 244; 687; 2129; Antisense;
TTAGTCCACCCAATGGCTGACAGTA
>HG-U133A: 206676_at; 27; 323; 2157; Antisense;
GCATCTTTAACACAACTCTTTGTTC
>HG-U133A: 206676_at; 591; 437; 2178; Antisense;
GTTCAAATGTACTATGGTCTCTTTT
>HG-U133A: 206676_at; 182; 641; 2244; Antisense;
TAATTTAACCCAGGCATGCAATGCT
>HG-U133A: 209395_at; 554; 307; 1427; Antisense;
GCTGTGGGGATAGTGAGGCATCGCA
>HG-U133A: 209395_at; 104; 501; 1432; Antisense;
GGGGATAGTGAGGCATCGCAATGTA
>HG-U133A: 209395_at; 445; 411; 1441; Antisense;
GAGGCATCGCAATGTAAGACTCGGG
>HG-U133A: 209395_at; 54; 325; 1444; Antisense;
GCATCGCAATGTAAGACTCGGGATT
>HG-U133A: 209395_at; 342; 363; 1454; Antisense;
GTAAGACTCGGGATTAGTACACACT
>HG-U133A: 209395_at; 433; 379; 1458; Antisense;
GACTCGGGATTAGTACACACTTGTT
>HG-U133A: 209395_at; 539; 513; 1464; Antisense;
GGATTAGTACACACTTGTTGATGAT
>HG-U133A: 209395_at; 587; 517; 1493; Antisense;
GGAAATGTTTACAGATCCCCAAGCC
>HG-U133A: 209395_at; 288; 117; 1495; Antisense;
AAATGTTTACAGATCCCCAAGCCTG
>HG-U133A: 209395_at; 466; 25; 1662; Antisense;
ACCTTCACTTAGGAACGTAATCGTG
>HG-U133A: 209395_at; 247; 661; 1671; Antisense;
TAGGAACGTAATCGTGTCCCCTATC
>HG-U133A: 209396_s_at; 468; 635; 1198; Antisense;
TCACCAATGCCATCAAGGATGCACT
>HG-U133A: 209396_s_at; 476; 167; 1211; Antisense;
CAAGGATGCACTCGCTGCAACGTAG
>HG-U133A: 209396_s_at; 630; 175; 1248; Antisense;
CACACAGCACGGGGGCCAAGGATGC
>HG-U133A: 209396_s_at; 688; 591; 1365; Antisense;
TGCAGAGGTCCACAACACACAGATT
>HG-U133A: 209396_s_at; 148; 177; 1382; Antisense;
CACAGATTTGAGCTCAGCCCTGGTG
>HG-U133A: 209396_s_at; 91; 255; 1547; Antisense;
CCCTAGCCCTCCTTATCAAAGGACA
>HG-U133A: 209396_s_at; 661; 151; 1565; Antisense;
AAGGACACCATTTTGGCAAGCTCTA
>HG-U133A: 209396_s_at; 218; 537; 1579; Antisense;
GGCAAGCTCTATCACCAAGGAGCCA
>HG-U133A: 209396_s_at; 118; 29; 1607; Antisense;
ATCCTACAAGACACAGTGACCATAC
>HG-U133A: 209396_s_at; 233; 55; 1621; Antisense;
AGTGACCATACTAATTATACCCCCT
>HG-U133A: 209396_s_at; 514; 337; 1646; Antisense;
GCAAAGCCAGCTTGAAACCTTCACT
>HG-U133A: 212306_at; 448; 695; 4357; Antisense;
TTCCCATTAACCTTTGCCAGTGTTA
>HG-U133A: 212306_at; 190; 573; 4493; Antisense;
TGCTACTTTGAGTTTTGTTTCGTAT
>HG-U133A: 212306_at; 458; 443; 4509; Antisense;
GTTTCGTATCATGTCCTATGCTAGA
>HG-U133A: 212306_at; 382; 157; 4566; Antisense;
AATTTGAACTACAGCTGGACTCCGT
>HG-U133A: 212306_at; 344; 307; 4579; Antisense;
GCTGGACTCCGTTTGTGTGATGGTG
>HG-U133A: 212306_at; 532; 423; 4603; Antisense;
GATACATGTCATTAGTTGCAACTTC
>HG-U133A: 212306_at; 267; 5; 4705; Antisense;
ATTGTCTATTGGTTATTGATCTTGC
>HG-U133A: 212306_at; 245; 569; 4748; Antisense;
TGTCCCTTCTATGATCCCTTAAGAA
>HG-U133A: 212306_at; 215; 141; 4772; Antisense;
AAGCTGCACCAAATCATCTGCCTGT
>HG-U133A: 212306_at; 660; 293; 4791; Antisense;
GCCTGTTTTTTCTTGATACTTACTG
>HG-U133A: 212306_at; 370; 683; 4843; Antisense;
TTTTGGTTTGTTTATATCTTTGTTG
>HG-U133A: 206207_at; 613; 171; 111; Antisense;
CAAAGGGCGACCACTTGTCTGTTTC
>HG-U133A: 206207_at; 385; 285; 207; Antisense;
CCAAGTGTGCTTTGGTCGTCGTGTG
>HG-U133A: 206207_at; 473; 475; 245; Antisense;
GTGAGTATGGGGCCTGGAAGCAGCA
>HG-U133A: 206207_at; 122; 359; 282; Antisense;
GAACATGCCCTTTCAGGATGGCCAA
>HG-U133A: 206207_at; 330; 559; 353; Antisense;
TGGTCAATGGCCAATCCTCTTACAC
>HG-U133A: 206207_at; 420; 45; 35; Antisense;
AGGAGACAACAATGTCCCTGCTACC
>HG-U133A: 206207_at; 706; 605; 367; Antisense;
TCCTCTTACACCTTTGACCATAGAA
>HG-U133A: 206207_at; 573; 421; 470; Antisense;
GATAACCAGACTTCATGTTGCCAAG
>HG-U133A: 206207_at; 428; 573; 485; Antisense;
TGTTGCCAAGGAATCCCTGTCTCTA
>HG-U133A: 206207_at; 489; 463; 503; Antisense;
GTCTCTACGTGAACTTGGGATTCCA
>HG-U133A: 206207_at; 504; 625; 82; Antisense;
TCTTTGTCTACTGGTTCTACTGTGA
>HG-U133A: 214683_s_at; 188; 531; 3331; Antisense;
GGAAAGGATTCTTGGACCTCTACCA
>HG-U133A: 214683_s_at; 178; 525; 3344; Antisense;
GGACCTCTACCAAAACATATGATAC
>HG-U133A: 214683_s_at; 258; 453; 3384; Antisense;
GTAAATATTTTCACCACGATCGATT
>HG-U133A: 214683_s_at; 681; 85; 3396; Antisense;
ACCACGATCGATTAGACTGGGATGA
>HG-U133A: 214683_s_at; 96; 587; 3418; Antisense;
TGAACACAGTTCTGCCGGCAGATAT
>HG-U133A: 214683_s_at; 664; 697; 3427; Antisense;
TTCTGCCGGCAGATATGTTTCAAGA
>HG-U133A: 214683_s_at; 693; 153; 3527; Antisense;
AATGTTGGAGTATGATCCAGCCAAA
>HG-U133A: 214683_s_at; 222; 185; 3544; Antisense;
CAGCCAAAAGAATTACTCTCAGAGA
>HG-U133A: 214683_s_at; 360; 569; 3618; Antisense;
TGTAATTGGACAGCTCTCTCGAAGA
>HG-U133A: 214683_s_at; 499; 373; 3626; Antisense;
GACAGCTCTCTCGAAGAGATCTTAC
>HG-U133A: 214683_s_at; 400; 507; 3773; Antisense;
GGTAATGAACATCTTTTTCAGTAAT
>HG-U133A: 202163_s_at; 168; 191; 1994; Antisense;
CAGATGGTCATCTGGATTCTCCCAC
>HG-U133A: 202163_s_at; 271; 701; 2045; Antisense;
TTCCTTCCAGCAAACCTTGAAACGT
>HG-U133A: 202163_s_at; 124; 475; 2087; Antisense;
GTGAGTAACAGGAATGTGTCTTTAA
>HG-U133A: 202163_s_at; 111; 659; 2117; Antisense;
TAGAGTGGTTACATTTAATCAGGCA
>HG-U133A: 202163_s_at; 653; 419; 2146; Antisense;
GATAATTTGGGTTCTTGAGTTGTTT
>HG-U133A: 202163_s_at; 98; 521; 2172; Antisense;
GGAGTAATATCCCACAACTGGGGTA
>HG-U133A: 202163_s_at; 224; 135; 2187; Antisense;
AACTGGGGTAGGAAGCTCAGGACTT
>HG-U133A: 202163_s_at; 55; 681; 2212; Antisense;
TTTTCTTTAAAGCTAGTCATTTCAA
>HG-U133A: 202163_s_at; 387; 125; 2315; Antisense;
AAAACTGGTAACTCACTCAAGTGAA
>HG-U133A: 202163_s_at; 485; 473; 2335; Antisense;
GTGAATGAATGGTCTTGCATTTTAA
>HG-U133A: 202163_s_at; 194; 115; 2359; Antisense;
AAAGCTTATGGGAAACTCAATTTGA
>HG-U133A: 221517_s_at; 157; 503; 1959; Antisense;
GGTGACTATGCTATTTCAGTTCGTA
>HG-U133A: 221517_s_at; 685; 455; 1981; Antisense;
GTAATGGACCTGAAAGTGGCAGCAA
>HG-U133A: 221517_s_at; 239; 539; 1998; Antisense;
GGCAGCAAGATTATGGTTCAGTTTC
>HG-U133A: 221517_s_at; 491; 435; 2013; Antisense;
GTTCAGTTTCCTCGTAACCAATGTA
>HG-U133A: 221517_s_at; 319; 597; 2024; Antisense;
TCGTAACCAATGTAAAGACCTTCCA
>HG-U133A: 221517_s_at; 343; 55; 2079; Antisense;
AGTCATCTTCGTGGGCCATTCAAAG
>HG-U133A: 221517_s_at; 98; 525; 2150; Antisense;
GGAGCTGCTTATGTCTGCACTTAGC
>HG-U133A: 221517_s_at; 261; 179; 2167; Antisense;
CACTTAGCCCTTGTCTACTATGATT
>HG-U133A: 221517_s_at; 141; 415; 2199; Antisense;
GATGTTTCCTAAAGAAGTTTCCAGA
>HG-U133A: 221517_s_at; 221; 423; 2271; Antisense;
GATAACTTCCAAAAGAGTGCTGTTT
>HG-U133A: 221517_s_at; 201; 163; 2480; Antisense;
AATATTCCTTCTTTGATGTTGACAT
>HG-U133A: 210766_s_at; 107; 513; 1732; Antisense;
GGTTCCATCAATGGTGAGCACCAGC
>HG-U133A: 210766_s_at; 97; 559; 1743; Antisense;
TGGTGAGCACCAGCCTGAATGCAGA
>HG-U133A: 210766_s_at; 276; 333; 1749; Antisense;
GCACCAGCCTGAATGCAGAAGCGCT
>HG-U133A: 210766_s_at; 115; 345; 1759; Antisense;
GAATGCAGAAGCGCTCCAGTATCTC
>HG-U133A: 210766_s_at; 639; 229; 1772; Antisense;
CTCCAGTATCTCCAAGGGTACCTTC
>HG-U133A: 210766_s_at; 457; 613; 1780; Antisense;
TCTCCAAGGGTACCTTCAGGCAGCC
>HG-U133A: 210766_s_at; 161; 53; 1805; Antisense;
AGTGTGACACTGCTTTAAACTGCAT
>HG-U133A: 210766_s_at; 129; 373; 1810; Antisense;
GACACTGCTTTAAACTGCATTTTTC
>HG-U133A: 210766_s_at; 559; 559; 1839; Antisense;
TGGGCTAAACCCAGATGGTTTCCTA
>HG-U133A: 210766_s_at; 525; 253; 1848; Antisense;
CCCAGATGGTTTCCTAGGAAATCAC
>HG-U133A: 210766_s_at; 215; 107; 1871; Antisense;
ACAGGCTTCTGAGCACAGCTGCATT
>HG-U133A: 203591_s_at; 578; 215; 2359; Antisense;
CTATGTGCTCCAGGGGGACCCAAGA
>HG-U133A: 203591_s_at; 569; 277; 2407; Antisense;
CCAGTCTGGCACCAGCGATCAGGTC
>HG-U133A: 203591_s_at; 694; 421; 2423; Antisense;
GATCAGGTCCTTTATGGGCAGCTGC
>HG-U133A: 203591_s_at; 654; 257; 2530; Antisense;
CCCCAAGTCCTATGAGAACCTCTGG
>HG-U133A: 203591_s_at; 364; 31; 2541; Antisense;
ATGAGAACCTCTGGTTCCAGGCCAG
>HG-U133A: 203591_s_at; 235; 519; 2602; Antisense;
GGAGGACGACTGTGTCTTTGGGCCA
>HG-U133A: 203591_s_at; 148; 505; 2652; Antisense;
GGATCCGGGTCCATGGGATGGAGGC
>HG-U133A: 203591_s_at; 181; 489; 2715; Antisense;
GGGCCTGCCTCTTAAAGGCCTGAGC
>HG-U133A: 203591_s_at; 362; 519; 2755; Antisense;
GGAGGGTCCATAAGCCCATGACTAA
>HG-U133A: 203591_s_at; 316; 227; 2856; Antisense;
CTCCCAGGCGATCTGCATACTTTAA
>HG-U133A: 203591_s_at; 550; 213; 2875; Antisense;
CTTTAAGGACCAGATCATGCTCCAT
>HG-U133A: 201487_at; 503; 379; 1288; Antisense;
GACTCAGCCTCTGGGATGGATTACT
>HG-U133A: 201487_at; 163; 505; 1345; Antisense;
GGTGAGAATGGCTACTTCCGGATCC
>HG-U133A: 201487_at; 702; 99; 1358; Antisense;
ACTTCCGGATCCGCAGAGGAACTGA
>HG-U133A: 201487_at; 549; 397; 1396; Antisense;
GAGAGCATAGCAGTGGCAGCCACAC
>HG-U133A: 201487_at; 227; 333; 1411; Antisense;
GCAGCCACACCAATTCCTAAATTGT
>HG-U133A: 201487_at; 565; 447; 1434; Antisense;
GTAGGGTATGCCTTCCAGTATTTCA
>HG-U133A: 201487_at; 591; 293; 1443; Antisense;
GCCTTCCAGTATTTCATAATGATCT
>HG-U133A: 201487_at; 706; 419; 1463; Antisense;
GATCTGCATCAGTTGTAAAGGGGAA
>HG-U133A: 201487_at; 277; 165; 1486; Antisense;
AATTGGTATATTCACAGACTGTAGA
>HG-U133A: 201487_at; 422; 381; 1502; Antisense;
GACTGTAGACTTTCAGCAGCAATCT
>HG-U133A: 201487_at; 161; 27; 1597; Antisense;
ACCTTTCAATCGGCCACTGGCCATT
>HG-U133A: 60084_at; 4; 439; 172; Antisense;
GTTATAATCTCTTCCTAGCTAATGG
>HG-U133A: 60084_at; 295; 223; 180; Antisense;
CTCTTCCTAGCTAATGGGCTTACTC
>HG-U133A: 60084_at; 623; 207; 182; Antisense;
CTTCCTAGCTAATGGGCTTACTCAA
>HG-U133A: 60084_at; 78; 657; 187; Antisense;
TAGCTAATGGGCTTACTCAAAGATT
>HG-U133A: 60084_at; 411; 563; 194; Antisense;
TGGGCTTACTCAAAGATTCACCACC
>HG-U133A: 60084_at; 686; 215; 30; Antisense;
CTAGCAATGATATTCTCAGTTGTTT
>HG-U133A: 60084_at; 334; 73; 32; Antisense;
AGCAATGATATTCTCAGTTGTTTCT
>HG-U133A: 60084_at; 607; 339; 33; Antisense;
GCAATGATATTCTCAGTTGTTTCTC
>HG-U133A: 60084_at; 460; 219; 44; Antisense;
CTCAGTTGTTTCTCTCTTGTGGTGC
>HG-U133A: 60084_at; 464; 695; 53; Antisense;
TTCTCTCTTGTGGTGCAGAGTTGCA
>HG-U133A: 60084_at; 258; 623; 56; Antisense;
TCTCTTGTGGTGCAGAGTTGCATTG
>HG-U133A: 60084_at; 73; 223; 57; Antisense;
CTCTTGTGGTGCAGAGTTGCATTGG
>HG-U133A: 60084_at; 354; 591; 66; Antisense;
TGCAGAGTTGCATTGGGTTTTCTAC
>HG-U133A: 60084_at; 196; 591; 74; Antisense;
TGCATTGGGTTTTCTACATTTTCCC
>HG-U133A: 60084_at; 587; 319; 75; Antisense;
GCATTGGGTTTTCTACATTTTCCCA
>HG-U133A: 60084_at; 476; 251; 96; Antisense;
CCCACTGAGTCTTCCCTGTTGTAAA
>HG-U133A: 202314_at; 448; 593; 2794; Antisense;
TGCTTCCTCTCTAGAATCCAATTAG
>HG-U133A: 202314_at; 516; 51; 2817; Antisense;
AGGGATGTTTGTTACTACTCATATT
>HG-U133A: 202314_at; 453; 37; 2877; Antisense;
ATGTGAGATCAGTGAACTCTGGTTT
>HG-U133A: 202314_at; 576; 359; 2890; Antisense;
GAACTCTGGTTTTAAGATAATCTGA
>HG-U133A: 202314_at; 465; 371; 2905; Antisense;
GATAATCTGAAACAAGGTCCTTGGG
>HG-U133A: 202314_at; 36; 129; 2936; Antisense;
AAAATTGGTCACATTCTGTAAAGCA
>HG-U133A: 202314_at; 444; 441; 2967; Antisense;
GTTTAGGAATCAACTTATCTCAAAT
>HG-U133A: 202314_at; 709; 659; 2982; Antisense;
TATCTCAAATTGTAACTCGGGGCCT
>HG-U133A: 202314_at; 214; 61; 2986; Antisense;
TCAAATTGTAACTCGGGGCCTAACT
>HG-U133A: 202314_at; 146; 689; 3047; Antisense;
TTCACTAGGTGATGCCAAAATATTT
>HG-U133A: 202314_at; 119; 573; 3117; Antisense;
TGTTAAACTCTAATTGTGAAGGCAG
>HG-U133A: 204244_s_at; 65; 561; 2194; Antisense;
TGAGGAACCCAATGAATGTGACTTC
>HG-U133A: 204244_s_at; 581; 73; 2220; Antisense;
AGAATATGGATAGTTTACCTTCTGG
>HG-U133A: 204244_s_at; 631; 339; 2355; Antisense;
GAATTTGTAGTTCACCGGTACAGTC
>HG-U133A: 204244_s_at; 299; 693; 2365; Antisense;
TTCACCGGTACAGTCTTTACTAGAC
>HG-U133A: 204244_s_at; 147; 625; 2378; Antisense;
TCTTTACTAGACTTGTTTCAGACTA
>HG-U133A: 204244_s_at; 287; 369; 2410; Antisense;
GAAATCAGAATTTTTGGGTTTCACA
>HG-U133A: 204244_s_at; 672; 617; 2500; Antisense;
TCTGTTAACAGCGTTTTTCTCGTCC
>HG-U133A: 204244_s_at; 320; 253; 2524; Antisense;
CCCTTCAACTTCTACATTTACTGGC
>HG-U133A: 204244_s_at; 599; 171; 2529; Antisense;
CAACTTCTACATTTACTGGCTTTTA
>HG-U133A: 204244_s_at; 704; 665; 2642; Antisense;
TTACCAGCTTTGTTTACAGACCCAA
>HG-U133A: 204244_s_at; 468; 641; 2738; Antisense;
TAAACTTGTGACTGGTCTTGTTTTA
>HG-U133A: 220890_s_at; 124; 83; 1059; Antisense;
AGCCCAAAGGTTTGCCCGAATGGAG
>HG-U133A: 220890_s_at; 614; 365; 1107; Antisense;
GAAACGCTCGCGAGAGGATGCTGGA
>HG-U133A: 220890_s_at; 244; 407; 1147; Antisense;
GAGGGTGCTATTGGTGTCAGGAACA
>HG-U133A: 220890_s_at; 391; 165; 1308; Antisense;
AATTGTGTCCAGAATGTGCTCAGCT
>HG-U133A: 220890_s_at; 443; 633; 1327; Antisense;
TCAGCTAATTCAGTATTCTTCCCCA
>HG-U133A: 220890_s_at; 117; 439; 1405; Antisense;
GTTACTGTTCTTCGACTTTGATTCC
>HG-U133A: 220890_s_at; 460; 377; 1418; Antisense;
GACTTTGATTCCTTGCTCATGACAT
>HG-U133A: 220890_s_at; 710; 199; 1440; Antisense;
CATGAGTAGGGTGTGCTCTTCTGTC
>HG-U133A: 220890_s_at; 612; 235; 1460; Antisense;
CTGTCACTTCACACAGACCTTTTGC
>HG-U133A: 220890_s_at; 72; 417; 1515; Antisense;
GATGATGCCCATGACCTGTAATTGT
>HG-U133A: 220890_s_at; 636; 9; 1567; Antisense;
ATTTAAACCATCTTGGCTTGTGCTT
>HG-U133A: 205033_s_at; 32; 301; 186; Antisense;
GCCCCGGAGCAGATTGCAGCGGACA
>HG-U133A: 205033_s_at; 330; 241; 190; Antisense;
CGGAGCAGATTGCAGCGGACATCCC
>HG-U133A: 205033_s_at; 538; 429; 197; Antisense;
GATTGCAGCGGACATCCCAGAAGTG
>HG-U133A: 205033_s_at; 494; 187; 214; Antisense;
CAGAAGTGGTTGTTTCCCTTGCATG
>HG-U133A: 205033_s_at; 129; 53; 218; Antisense;
AGTGGTTGTTTCCCTTGCATGGGAC
>HG-U133A: 205033_s_at; 66; 513; 221; Antisense;
GGTTGTTTCCCTTGCATGGGACGAA
>HG-U133A: 205033_s_at; 346; 437; 225; Antisense;
GTTTCCCTTGCATGGGACGAAAGCT
>HG-U133A: 205033_s_at; 564; 325; 234; Antisense;
GCATGGGACGAAAGCTTGGCTCCAA
>HG-U133A: 205033_s_at; 673; 381; 240; Antisense;
GACGAAAGCTTGGCTCCAAAGCATC
>HG-U133A: 205033_s_at; 162; 295; 64; Antisense;
GCCTAGCTAGAGGATCTGTGACCCC
>HG-U133A: 205033_s_at; 524; 217; 66; Antisense;
CTAGCTAGAGGATCTGTGACCCCAG
>HG-U133A: 207269_at; 491; 331; 162; Antisense;
GCAGCGTGGGCCAGAAGACCAGGAC
>HG-U133A: 207269_at; 508; 127; 211; Antisense;
AAAAGCTCTGCTCTTCAGGTTTCAG
>HG-U133A: 207269_at; 411; 543; 235; Antisense;
GGCTCAACAAGGGGCATGGTCTGCT
>HG-U133A: 207269_at; 642; 561; 300; Antisense;
TGGGAACTGCCTCATTGGTGGTGTG
>HG-U133A: 207269_at; 137; 477; 322; Antisense;
GTGAGTTTCACATACTGCTGCACGC
>HG-U133A: 207269_at; 140; 311; 338; Antisense;
GCTGCACGCGTGTCGATTAACGTTC
>HG-U133A: 207269_at; 709; 427; 352; Antisense;
GATTAACGTTCTGCTGTCCAAGAGA
>HG-U133A: 207269_at; 394; 629; 380; Antisense;
TCATGCTGGGAACGCCATCATCGGT
>HG-U133A: 207269_at; 295; 503; 402; Antisense;
GGTGGTGTTAGCTTCACATGCTTCT
>HG-U133A: 207269_at; 66; 179; 416; Antisense;
CACATGCTTCTGCAGCTGAGCTTGC
>HG-U133A: 207269_at; 433; 185; 59; Antisense;
CAGCCATGAGGATTATCGCCCTCCT
>HG-U133A: 215501_s_at; 344; 565; 508; Antisense;
TGTGGGAAGGGGCTTCTCATCCACT
>HG-U133A: 215501_s_at; 488; 277; 560; Antisense;
CCATCGTCATCGCTTACTTGATGAA
>HG-U133A: 215501_s_at; 510; 353; 582; Antisense;
GAAGCACACTCGGATGACCATGACT
>HG-U133A: 215501_s_at; 240; 517; 593; Antisense;
GGATGACCATGACTGATGCTTATAA
>HG-U133A: 215501_s_at; 537; 171; 624; Antisense;
CAAAGGCAAACGACCAATTATCTCC
>HG-U133A: 215501_s_at; 5; 501; 666; Antisense;
GGGGCAGTTGCTAGAGTTCGAGGAA
>HG-U133A: 215501_s_at; 481; 581; 707; Antisense;
TGACACCGAGAATCCTTACACCAAA
>HG-U133A: 215501_s_at; 603; 643; 723; Antisense;
TACACCAAAGCTGATGGGCGTGGAG
>HG-U133A: 215501_s_at; 695; 545; 739; Antisense;
GGCGTGGAGACGGTTGTGTGACAAT
>HG-U133A: 215501_s_at; 556; 151; 776; Antisense;
AAGGATTGCTGCTCTCCATTAGGAG
>HG-U133A: 215501_s_at; 399; 577; 931; Antisense;
TGATGCCATTGAGATTCACCTCCCA
>HG-U133A: 221563_at; 566; 67; 2147; Antisense;
AGACATTGAATCACCAAGGCCTGGG
>HG-U133A: 221563_at; 274; 167; 2161; Antisense;
CAAGGCCTGGGATCAACCTGGGCTG
>HG-U133A: 221563_at; 244; 139; 2208; Antisense;
AACCAAACCAAGCCCTGTTGTGCTC
>HG-U133A: 221563_at; 598; 421; 2249; Antisense;
GATCAGGGCAGCTTAAGTGGTCTAA
>HG-U133A: 221563_at; 298; 483; 2265; Antisense;
GTGGTCTAAGAATCCTTCAGGCATT
>HG-U133A: 221563_at; 454; 515; 2305; Antisense;
GGATACCTTTGATTTTGTGTGTTTC
>HG-U133A: 221563_at; 416; 481; 2321; Antisense;
GTGTGTTTCATGCTCTGGATTTTTT
>HG-U133A: 221563_at; 573; 531; 2392; Antisense;
GGAACTGACCATTATATGCCTTCAC
>HG-U133A: 221563_at; 72; 205; 2401; Antisense;
CATTATATGCCTTCACTGGCTTCTT
>HG-U133A: 221563_at; 312; 15; 2532; Antisense;
ATATATCAAATACTTTCCTTCCCAC
>HG-U133A: 221563_at; 419; 437; 2595; Antisense;
GTTACAGTGCCATAAACCTTGTTAC
>HG-U133A: 201016_at; 88; 457; 1297; Antisense;
GTAAGCTTAGTAGTTGCAGAAATTG
>HG-U133A: 201016_at; 497; 361; 1321; Antisense;
GAACACTAGGTGGCACTCAGTTATC
>HG-U133A: 201016_at; 349; 485; 1330; Antisense;
GTGGCACTCAGTTATCTTAACAGGG
>HG-U133A: 201016_at; 327; 97; 1360; Antisense;
ACTGATACAATTGTTGACTTTTCTT
>HG-U133A: 201016_at; 18; 195; 1386; Antisense;
TACTATGTGTAAGAAATACCCCAAA
>HG-U133A: 201016_at; 551; 363; 1398; Antisense;
GAAATACCCCAAACATGAAAAGATT
>HG-U133A: 201016_at; 233; 3; 1420; Antisense;
ATTGTTTTGATCATATGCATGTATG
>HG-U133A: 201016_at; 370; 347; 1500; Antisense;
GAAGTCATATACATGTAAGCTACAA
>HG-U133A: 201016_at; 348; 363; 1605; Antisense;
GAAAAGCCTTTTTCAACATATCCCT
>HG-U133A: 201016_at; 59; 681; 1613; Antisense;
TTTTTCAACATATCCCTAAGCTAAG
>HG-U133A: 201016_at; 519; 171; 1655; Antisense;
CAACTCAGTGAAAAGATGGTCTCCA
>HG-U133A: 218696_at; 331; 353; 3778; Antisense;
GAAGAAGGAAAGTCCCCCTGTGTGG
>HG-U133A: 218696_at; 278; 535; 3904; Antisense;
GGAATCTGCACTATTTTGGAGGACA
>HG-U133A: 218696_at; 474; 37; 3954; Antisense;
ATGTCCGTAGTTTTATAGTCCTATT
>HG-U133A: 218696_at; 12; 669; 3967; Antisense;
TATAGTCCTATTTGTAGCATTCAAT
>HG-U133A: 218696_at; 59; 17; 3990; Antisense;
ATAGCTTTATTCCTTAGATGGTTCT
>HG-U133A: 218696_at; 344; 405; 4006; Antisense;
GATGGTTCTAGGGTGGGTTTACAGC
>HG-U133A: 218696_at; 114; 101; 4146; Antisense;
ACTAACTTCTTCAACTATGGACTTT
>HG-U133A: 218696_at; 188; 571; 4202; Antisense;
TGTAATCCTGTAGGTTGGTACTTCC
>HG-U133A: 218696_at; 126; 609; 4224; Antisense;
TCCCCCAAACTGATTATAGGTAACA
>HG-U133A: 218696_at; 298; 509; 4242; Antisense;
GGTAACAGTTTAATCATCTCACTTG
>HG-U133A: 218696_at; 117; 25; 4254; Antisense;
ATCATCTCACTTGCTAACATGTTTT
>HG-U133A: 206871_at; 320; 471; 351; Antisense;
GTGCAGCGCATCTTCGAAAACGGCT
>HG-U133A: 206871_at; 378; 89; 379; Antisense;
ACCCCGTAAACTTGCTCAACGACAT
>HG-U133A: 206871_at; 207; 639; 394; Antisense;
TCAACGACATCGTGATTCTCCAGCT
>HG-U133A: 206871_at; 685; 87; 432; Antisense;
ACCATCAACGCCAACGTGCAGGTGG
>HG-U133A: 206871_at; 18; 565; 526; Antisense;
TGGGCAGGAACCGTGGGATCGCCAG
>HG-U133A: 206871_at; 54; 327; 557; Antisense;
GCAGGAGCTCAACGTGACGGTGGTG
>HG-U133A: 206871_at; 228; 169; 566; Antisense;
CAACGTGACGGTGGTGACGTCCCTC
>HG-U133A: 206871_at; 693; 253; 663; Antisense;
CCCTTGGTCTGCAACGGGCTAATCC
>HG-U133A: 206871_at; 153; 245; 677; Antisense;
CGGGCTAATCCACGGAATTGCCTCC
>HG-U133A: 206871_at; 559; 297; 747; Antisense;
GCCCCGGTGGCACAGTTTGTAAACT
>HG-U133A: 206871_at; 534; 703; 763; Antisense;
TTGTAAACTGGATCGACTCTATCAT
>HG-U133A: 203358_s_at; 214; 113; 2117; Antisense;
AAATTCGTTTTGCAAATCATTCGGT
>HG-U133A: 203358_s_at; 13; 115; 2130; Antisense;
AAATCATTCGGTAAATCCAAACTGC
>HG-U133A: 203358_s_at; 446; 421; 2182; Antisense;
GATCACAGGATAGGTATTTTTGCCA
>HG-U133A: 203358_s_at; 149; 683; 2199; Antisense;
TTTTGCCAAGAGAGCCATCCAGACT
>HG-U133A: 203358_s_at; 461; 277; 2213; Antisense;
CCATCCAGACTGGCGAAGAGCTGTT
>HG-U133A: 203358_s_at; 57; 365; 2337; Antisense;
GAAACAGCTGCCTTAGCTTCAGGAA
>HG-U133A: 203358_s_at; 291; 231; 2344; Antisense;
CTGCCTTAGCTTCAGGAACCTCGAG
>HG-U133A: 203358_s_at; 407; 629; 2355; Antisense;
TCAGGAACCTCGAGTACTGTGGGCA
>HG-U133A: 203358_s_at; 564; 293; 2473; Antisense;
GCCTTCTCACCAGCTGCAAAGTGTT
>HG-U133A: 203358_s_at; 639; 173; 2489; Antisense;
CAAAGTGTTTTGTACCAGTGAATTT
>HG-U133A: 203358_s_at; 529; 327; 2524; Antisense;
GCAGTATGGTACATTTTTCAACTTT
>HG-U133A: 212333_at; 189; 425; 2197; Antisense;
GATAGCACATTCAGTAGCCTTATTT
>HG-U133A: 212333_at; 306; 651; 2233; Antisense;
TACTGTATCATATGCTCAACTCTGA
>HG-U133A: 212333_at; 513; 137; 2259; Antisense;
AACCTTGAACACGGCCAAAATCCAT
>HG-U133A: 212333_at; 640; 165; 2348; Antisense;
CAATTCAAACTGACCTGCATCCATC
>HG-U133A: 212333_at; 472; 233; 2362; Antisense;
CTGCATCCATCCAAAACAAATTCCT
>HG-U133A: 212333_at; 251; 399; 2453; Antisense;
GAGTTAATACCACTGGCTCAGCAAA
>HG-U133A: 212333_at; 513; 45; 2563; Antisense;
AGGAGGCCCTTTATTATTGCTGCAG
>HG-U133A: 212333_at; 113; 297; 2597; Antisense;
GCCTGGCTGAGTTGATGTTTTACAT
>HG-U133A: 212333_at; 620; 621; 2622; Antisense;
TCTCCCTTACTGAAATCTACATGAC
>HG-U133A: 212333_at; 649; 417; 2649; Antisense;
GATGCTTCTTGCTGGGTTTTTGTAC
>HG-U133A: 212333_at; 292; 41; 2703; Antisense;
ATGGCTGGAGGTGTGCTTTGTGTGA
>HG-U133A: 208988_at; 505; 433; 6399; Antisense;
GTTGCTGATTTAGAGTCAATCTCCA
>HG-U133A: 208988_at; 25; 661; 6409; Antisense;
TAGAGTCAATCTCCAATGTTGTGCT
>HG-U133A: 208988_at; 205; 495; 6464; Antisense;
GGGATAAGTCTTATGCTATCTCAGT
>HG-U133A: 208988_at; 597; 661; 6475; Antisense;
TATGCTATCTCAGTTGACACATTGA
>HG-U133A: 208988_at; 566; 189; 6485; Antisense;
CAGTTGACACATTGAGGTTATTTTG
>HG-U133A: 208988_at; 314; 353; 6524; Antisense;
GAAGCTAGTTGGACTTTGTTTTGTT
>HG-U133A: 208988_at; 35; 575; 6545; Antisense;
TGTTTTCCAAAAGTTCTCCACTATT
>HG-U133A: 208988_at; 398; 145; 6555; Antisense;
AAGTTCTCCACTATTGGTTTTAGAG
>HG-U133A: 208988_at; 594; 73; 6582; Antisense;
AGCAAGGACATCTTTCCTCTGACAC
>HG-U133A: 208988_at; 48; 95; 6605; Antisense;
ACGTGGGAATGGGTGATATTTGTGT
>HG-U133A: 208988_at; 682; 365; 6656; Antisense;
GAAATAGCCTCCAATGGGAAATATT
>HG-U133A: 208989_s_at; 24; 523; 3578; Antisense;
GGAGAGACCCCTTCAGAGCAGGGAT
>HG-U133A: 208989_s_at; 163; 383; 3583; Antisense;
GACCCCTTCAGAGCAGGGATTGTGC
>HG-U133A: 208989_s_at; 126; 51; 3597; Antisense;
AGGGATTGTGCCGGGAGAGTGCCTC
>HG-U133A: 208989_s_at; 15; 673; 3626; Antisense;
TTTGGGACATTTCATCCACAGAAAT
>HG-U133A: 208989_s_at; 370; 189; 3644; Antisense;
CAGAAATTTCCAAGCCAATGGTTTC
>HG-U133A: 208989_s_at; 73; 189; 3770; Antisense;
CAGCAGATGGACCATGCCCTTGCTG
>HG-U133A: 208989_s_at; 225; 361; 3862; Antisense;
GAAAGAAGTGTCTCTGTTGGGGGAC
>HG-U133A: 208989_s_at; 539; 695; 3878; Antisense;
TTGGGGGACAGAGGAACCTGGGGAG
>HG-U133A: 208989_s_at; 390; 325; 3909; Antisense;
GCATGTCCTACAATCTGCTCTTAGA
>HG-U133A: 208989_s_at; 238; 221; 3926; Antisense;
CTCTTAGACACGGCCTTGCCAGGAG
>HG-U133A: 208989_s_at; 124; 685; 3929; Antisense;
TTAGACACGGCCTTGCCAGGAGAGC
>HG-U133A: 218432_at; 403; 365; 1811; Antisense;
GAAACTATGTGACTCATTCTGTGAA
>HG-U133A: 218432_at; 138; 145; 1836; Antisense;
AAGACTTCTTGCAGTTGTGAGTTAT
>HG-U133A: 218432_at; 611; 41; 1886; Antisense;
AGGCTAATCCATTTAGTGATTCCTA
>HG-U133A: 218432_at; 478; 357; 1951; Antisense;
GAACGCTAGTGGTTTGTCCTTAGAC
>HG-U133A: 218432_at; 628; 213; 1998; Antisense;
CTTTATCGCTAAGACCTTGACTTTA
>HG-U133A: 218432_at; 260; 113; 2022; Antisense;
AAATTTTTCATCACTACAACCTTGA
>HG-U133A: 218432_at; 272; 641; 2050; Antisense;
TAATTTCAGGTCTTCAACATGATGA
>HG-U133A: 218432_at; 138; 463; 2093; Antisense;
GTCTTCAACACTATGCGCTTTATCA
>HG-U133A: 218432_at; 348; 597; 2106; Antisense;
TGCGCTTTATCATATTATTCACAGA
>HG-U133A: 218432_at; 54; 709; 2217; Antisense;
TTGTAAATACTGCTTCTGTTTTGTT
>HG-U133A: 218432_at; 88; 443; 2239; Antisense;
GTTTCTCCTTTATACACTTGACTGT
>HG-U133A: 211307_s_at; 436; 603; 381; Antisense;
TCCATCCACCAAGATTACACGACGC
>HG-U133A: 211307_s_at; 288; 111; 397; Antisense;
ACACGACGCAGAACTTGATCCGCAT
>HG-U133A: 211307_s_at; 116; 543; 446; Antisense;
GGCTCTCTTGGCCATACTGGTTGAA
>HG-U133A: 211307_s_at; 557; 707; 473; Antisense;
TTGGCACAGCCATACGGCACTGAAC
>HG-U133A: 211307_s_at; 370; 537; 488; Antisense;
GGCACTGAACAAGGAAGCCTCGGCA
>HG-U133A: 211307_s_at; 196; 533; 500; Antisense;
GGAAGCCTCGGCAGATGTGGCTGAA
>HG-U133A: 211307_s_at; 370; 63; 512; Antisense;
AGATGTGGCTGAACCGAGCTGGAGC
>HG-U133A: 211307_s_at; 650; 309; 519; Antisense;
GCTGAACCGAGCTGGAGCCAACAGA
>HG-U133A: 211307_s_at; 80; 525; 532; Antisense;
GGAGCCAACAGATGTGTCAGCCAGG
>HG-U133A: 211307_s_at; 63; 459; 547; Antisense;
GTCAGCCAGGATTGACCTTTGCACG
>HG-U133A: 211307_s_at; 197; 279; 552; Antisense;
CCAGGATTGACCTTTGCACGAACAC
>HG-U133A: 206759_at; 344; 699; 1110; Antisense;
TTCCGCGGAGTCCATGGGACCTGAT
>HG-U133A: 206759_at; 694; 249; 1113; Antisense;
CGCGGAGTCCATGGGACCTGATTCA
>HG-U133A: 206759_at; 622; 401; 1117; Antisense;
GAGTCCATGGGACCTGATTCAAGAC
>HG-U133A: 206759_at; 74; 467; 1119; Antisense;
GTCCATGGGACCTGATTCAAGACCA
>HG-U133A: 206759_at; 601; 573; 1131; Antisense;
TGATTCAAGACCAGACCCTGACGGC
>HG-U133A: 206759_at; 228; 39; 941; Antisense;
ATGTGGACTACAGCAACTGGGCTCC
>HG-U133A: 206759_at; 473; 555; 944; Antisense;
TGGACTACAGCAACTGGGCTCCAGG
>HG-U133A: 206759_at; 45; 101; 947; Antisense;
ACTACAGCAACTGGGCTCCAGGGGA
>HG-U133A: 206759_at; 53; 51; 989; Antisense;
AGGGCGAGGACTGCGTGATGATGCG
>HG-U133A: 206759_at; 171; 547; 991; Antisense;
GGCGAGGACTGCGTGATGATGCGGG
>HG-U133A: 206759_at; 205; 45; 995; Antisense;
AGGACTGCGTGATGATGCGGGGCTC
>HG-U133A: 203561_at; 212; 597; 1710; Antisense;
TGCTGGGATGACCAGCATCAGCCCC
>HG-U133A: 203561_at; 634; 697; 1796; Antisense;
TTCTGCCTTCTCCATGCTGAGAACA
>HG-U133A: 203561_at; 590; 115; 1821; Antisense;
AAATCACCTATTCACTGCTTATGCA
>HG-U133A: 203561_at; 646; 319; 1837; Antisense;
GCTTATGCAGTCGGAAGCTCCAGAA
>HG-U133A: 203561_at; 134; 351; 1859; Antisense;
GAAGAACAAAGAGCCCAATTACCAG
>HG-U133A: 203561_at; 262; 357; 1883; Antisense;
GAACCACATTAAGTCTCCATTGTTT
>HG-U133A: 203561_at; 589; 275; 1899; Antisense;
CCATTGTTTTGCCTTGGGATTTGAG
>HG-U133A: 203561_at; 488; 143; 1979; Antisense;
AAGACGAAGGGATGCTGCAGTTCCA
>HG-U133A: 203561_at; 168; 507; 2076; Antisense;
GGTCCCAAATGACTGACTGCACCTT
>HG-U133A: 203561_at; 459; 29; 2144; Antisense;
ATCCACACAGCCAATACAATTAGTC
>HG-U133A: 203561_at; 578; 363; 2200; Antisense;
GAAAGACGCTATGTTACAGGTTACA
>HG-U133A: 210992_x_at; 448; 529; 482; Antisense;
GGACAAGCCTCTGGTCAAGGTCACA
>HG-U133A: 210992_x_at; 28; 201; 631; Antisense;
CATCCAAGCCTGTGACCATCACTGT
>HG-U133A: 210992_x_at; 411; 611; 662; Antisense;
TCCCAGCTCTTCACCGATGGGGATC
>HG-U133A: 210992_x_at; 390; 559; 697; Antisense;
TGGTCACTGGGATTGCTGTAGCGGC
>HG-U133A: 210992_x_at; 441; 657; 715; Antisense;
TAGCGGCCATTGTTGCTGCTGTAGT
>HG-U133A: 210992_x_at; 326; 139; 762; Antisense;
AAGCGGATTTCAGCCAATTCCACTG
>HG-U133A: 210992_x_at; 626; 601; 780; Antisense;
TCCACTGATCCTGTGAAGGCTGCCC
>HG-U133A: 210992_x_at; 294; 591; 800; Antisense;
TGCCCAATTTGAGATGCTTTCCTGC
>HG-U133A: 210992_x_at; 11; 333; 823; Antisense;
GCAGCCACCTGGACGTCAAATGATT
>HG-U133A: 210992_x_at; 159; 131; 892; Antisense;
AACAGCTGACGGCGGCTACATGACT
>HG-U133A: 210992_x_at; 224; 647; 946; Antisense;
TAAAAACATCTACCTGACTCTTCCT
>HG-U133A: 211395_x_at; 449; 471; 384; Antisense;
GTGCATCTGACTGTGCTTTCTGAGT
>HG-U133A: 211395_x_at; 33; 71; 449; Antisense;
AGAAACCATCGTGCTGAGGTGCCAC
>HG-U133A: 211395_x_at; 337; 45; 481; Antisense;
AGGACAAGCCTCTGGTCAAGGTCAT
>HG-U133A: 211395_x_at; 695; 103; 610; Antisense;
ACATAGGCTACACGCTGTACTCATC
>HG-U133A: 211395_x_at; 27; 201; 631; Antisense;
CATCCAAGCCTGTGACCATCACTGT
>HG-U133A: 211395_x_at; 410; 611; 662; Antisense;
TCCCAGCTCTTCACCGATGGGGATC
>HG-U133A: 211395_x_at; 601; 413; 677; Antisense;
GATGGGGATCATTGTGGCTGTGGTC
>HG-U133A: 211395_x_at; 389; 559; 697; Antisense;
TGGTCACTGGGATTGCTGTAGCGGC
>HG-U133A: 211395_x_at; 440; 657; 715; Antisense;
TAGCGGCCATTGTTGCTGCTGTAGT
>HG-U133A: 211395_x_at; 158; 131; 840; Antisense;
AACAGCTGACGGCGGCTACATGACT
>HG-U133A: 211395_x_at; 223; 647; 894; Antisense;
TAAAAACATCTACCTGACTCTTCCT
>HG-U133A: 204007_at; 518; 459; 1411; Antisense;
GTCTTCCAGGGGACTCTATCAGAAC
>HG-U133A: 204007_at; 46; 417; 1459; Antisense;
GATGAGCCCTCTAATGCTAGGAGTA
>HG-U133A: 204007_at; 415; 491; 1502; Antisense;
GGGACTGAGGATTGGGGTGGGGGTG
>HG-U133A: 204007_at; 121; 109; 1543; Antisense;
ACAGAACAAACCCTGTGTCACTGTC
>HG-U133A: 204007_at; 448; 479; 1557; Antisense;
GTGTCACTGTCCCAAGTTAAGCTAA
>HG-U133A: 204007_at; 48; 475; 1582; Antisense;
GTGAACAGAACTATCTCAGCATCAG
>HG-U133A: 204007_at; 222; 619; 1741; Antisense;
TCTGCTTCAATGTCTAGTTCCTGTA
>HG-U133A: 204007_at; 545; 607; 1759; Antisense;
TCCTGTATAGCTTTGTTCATTGCAT
>HG-U133A: 204007_at; 624; 95; 1823; Antisense;
ACTGAGCTTCACTGAGTTACGCTGT
>HG-U133A: 204007_at; 601; 625; 1853; Antisense;
TTTCAAATCCTTCTTCAGTCAGTTC
>HG-U133A: 204007_at; 372; 125; 1918; Antisense;
AAAAAGCTTTAGCTGTCTCCTGTTT
>HG-U133A: 205237_at; 110; 511; 1011; Antisense;
GGTATCAACTGGAGTGCGGCGAAGG
>HG-U133A: 205237_at; 521; 269; 1099; Antisense;
CCTCCACATGCACCTGCTAGTGGGG
>HG-U133A: 205237_at; 516; 89; 1131; Antisense;
ACCCACAAGCGCTGCGTCGTGGAAG
>HG-U133A: 205237_at; 411; 403; 738; Antisense;
GAGGGCAACCACCAGTTTGCTAAGT
>HG-U133A: 205237_at; 107; 489; 827; Antisense;
GGGCAGTGCGGGTAATTCTCTAACG
>HG-U133A: 205237_at; 383; 663; 842; Antisense;
TTCTCTAACGGGCCACAACAACAAC
>HG-U133A: 205237_at; 595; 475; 897; Antisense;
GTGAGTTCTTCGAATTGTGCTGAGA
>HG-U133A: 205237_at; 4; 603; 925; Antisense;
TCCAGGGAGCCTGGTGGTACGCCGA
>HG-U133A: 205237_at; 175; 381; 948; Antisense;
GACTGTCATGCTTCAAACCTCAATG
>HG-U133A: 205237_at; 498; 219; 966; Antisense;
CTCAATGGTCTCTACCTCATGGGAC
>HG-U133A: 205237_at; 345; 39; 984; Antisense;
ATGGGACCCCATGAGAGCTATGCCA
>HG-U133A: 205119_s_at; 657; 111; 1071; Antisense;
ACACAGCTACCAATTCTACTTTACC
>HG-U133A: 205119_s_at; 694; 235; 1135; Antisense;
CTGGGGGACACTTTCGAGCTCCCAG
>HG-U133A: 205119_s_at; 77; 77; 1164; Antisense;
AGCTTCGTCTCACCTTGAGTTAGGC
>HG-U133A: 205119_s_at; 601; 41; 1185; Antisense;
AGGCTGAGCACAGGCATTTCCTGCT
>HG-U133A: 205119_s_at; 233; 11; 1211; Antisense;
ATTTTAGGATTACCCACTCATCAGA
>HG-U133A: 205119_s_at; 374; 35; 713; Antisense;
ATGTCCATCGTTGCTGTCAGTTATG
>HG-U133A: 205119_s_at; 645; 319; 739; Antisense;
GCTTATTGCCACCAAGATCCACAAG
>HG-U133A: 205119_s_at; 65; 41; 766; Antisense;
AGGCTTGATTAAGTCCAGTCGTCCC
>HG-U133A: 205119_s_at; 183; 503; 847; Antisense;
GGTGGTGGCCCTTATAGCCACAGTC
>HG-U133A: 205119_s_at; 201; 195; 918; Antisense;
CAGTGGATGTGACAAGTGCCCTGGC
>HG-U133A: 205119_s_at; 344; 137; 962; Antisense;
AACCCCATGCTCTATGTCTTCATGG
>HG-U133A: 209864_at; 235; 73; 1591; Antisense;
AGAAGAGCCCTGTTGGTGCTTTACC
>HG-U133A: 209864_at; 91; 403; 1624; Antisense;
GAGTCTCCCGAGGACACAAACAGGC
>HG-U133A: 209864_at; 112; 479; 1659; Antisense;
GTGTAGGGAGAGTTCTTTCCTGTTT
>HG-U133A: 209864_at; 194; 265; 1710; Antisense;
CCGGAAGGCCACTCATGGCCATGCC
>HG-U133A: 209864_at; 477; 631; 1722; Antisense;
TCATGGCCATGCCAGGAGCTTTCTC
>HG-U133A: 209864_at; 32; 205; 1756; Antisense;
CATAAACGATCTCTTGAGTCTCTTT
>HG-U133A: 209864_at; 460; 671; 1805; Antisense;
TATTCCACCCTTTCTGGTGTCTATA
>HG-U133A: 209864_at; 619; 579; 1838; Antisense;
TGAGAGACCCTGGACGTTTTTCTGC
>HG-U133A: 209864_at; 189; 545; 2005; Antisense;
GGCTGTATGAAACTTGACGGCGCTT
>HG-U133A: 209864_at; 704; 381; 2020; Antisense;
GACGGCGCTTTTGTAAGGTGCCACC
>HG-U133A: 209864_at; 117; 319; 2111; Antisense;
GCTATTGATGTACACTTCGCAACGG
>HG-U133A: 204299_at; 94; 475; 2334; Antisense;
GTGACTTGACATGTCCAATTTCATT
>HG-U133A: 204299_at; 693; 127; 2384; Antisense;
AAAATCTCAGATTGCTTGCTTACAG
>HG-U133A: 204299_at; 465; 555; 2425; Antisense;
TGGACAAACGATTCCTTTTAGAGGA
>HG-U133A: 204299_at; 125; 441; 2468; Antisense;
GTTTTAGTAATCTAGGCTTTGCCTG
>HG-U133A: 204299_at; 691; 513; 2543; Antisense;
GGATTGATTCTAGAACCTTTGTATA
>HG-U133A: 204299_at; 190; 425; 2571; Antisense;
GATAGTATTTCTAACTTTCATTTCT
>HG-U133A: 204299_at; 452; 437; 2614; Antisense;
GTTCATGTTCTGCTATGCAATCGTT
>HG-U133A: 204299_at; 539; 681; 2658; Antisense;
TTTTTTTAGATTTTCCTGGATGTAT
>HG-U133A: 204299_at; 523; 447; 2714; Antisense;
GTAGCAGTAGTTTACAGTTCTAGCA
>HG-U133A: 204299_at; 474; 115; 2862; Antisense;
AAAACAAGACCCAGCTTATTTTCTG
>HG-U133A: 204299_at; 157; 79; 2874; Antisense;
AGCTTATTTTCTGCTTGCTGTAAAT
>HG-U133A: 206095_s_at; 280; 5; 1254; Antisense;
ATTGAGCCCTTACTGTGGGCAAATC
>HG-U133A: 206095_s_at; 95; 423; 1305; Antisense;
GATAATTCCCTTATTCAGTAAATGT
>HG-U133A: 206095_s_at; 482; 115; 1324; Antisense;
AAATGTCTACTGAGCACAATCTAGT
>HG-U133A: 206095_s_at; 300; 473; 1347; Antisense;
GTGAATCATTACAGTATGGCCTCAT
>HG-U133A: 206095_s_at; 340; 123; 1398; Antisense;
AACAATATTTTACACCATTCGTATC
>HG-U133A: 206095_s_at; 388; 457; 1460; Antisense;
GTAATTGTGTGGTTATCTGCCATTT
>HG-U133A: 206095_s_at; 498; 147; 1487; Antisense;
AAGTATCCAGTATTTGATCACATTA
>HG-U133A: 206095_s_at; 290; 239; 1549; Antisense;
CTGGTTTATTGTGCAGTGACTGTAA
>HG-U133A: 206095_s_at; 329; 231; 1603; Antisense;
CTGCCTCACCAAACACATGCTAGGA
>HG-U133A: 206095_s_at; 528; 199; 1618; Antisense;
CATGCTAGGATATAACCCCCAAAAT
>HG-U133A: 206095_s_at; 709; 373; 1707; Antisense;
GACAGAGAGCTGTTATCCTAACTGA
>HG-U133A: 203853_s_at; 369; 699; 5516; Antisense;
TTCCCCATCTGGGCCTTCATAAAAT
>HG-U133A: 203853_s_at; 189; 31; 5539; Antisense;
ATGCAGGGGAAGCCAGACTGGTCTC
>HG-U133A: 203853_s_at; 586; 559; 5557; Antisense;
TGGTCTCAGGAGCGCTAAAGCCCTT
>HG-U133A: 203853_s_at; 370; 301; 5609; Antisense;
GCCCTGCTGTTTAGGACCTGGGACC
>HG-U133A: 203853_s_at; 405; 239; 5626; Antisense;
CTGGGACCACAATGGGGTACCTGCC
>HG-U133A: 203853_s_at; 280; 609; 5658; Antisense;
TCCCCAAGAGATCCAGGCTGTCATG
>HG-U133A: 203853_s_at; 253; 431; 5712; Antisense;
GTTGGCTACTTGTGTCTTGAAATCT
>HG-U133A: 203853_s_at; 355; 53; 5789; Antisense;
AGTGGAAGCCCAGTCTTGAGTTCTT
>HG-U133A: 203853_s_at; 592; 399; 5806; Antisense;
GAGTTCTTGTCTTGTTACCATTTAA
>HG-U133A: 203853_s_at; 539; 53; 5955; Antisense;
AGTGGGTCATGTTTTTGCTGTGGTG
>HG-U133A: 203853_s_at; 578; 371; 5979; Antisense;
GACACATGGTACAGGCTTGGAGCTT
>HG-U133A: 213049_at; 242; 529; 1624; Antisense;
GGAACATAACCCAGGAGTCTAAGTT
>HG-U133A: 213049_at; 316; 91; 1667; Antisense;
ACTGAACTTGCAGGTCCAGGTTGGT
>HG-U133A: 213049_at; 649; 277; 1682; Antisense;
CCAGGTTGGTATACATTCCACCCTC
>HG-U133A: 213049_at; 511; 619; 1697; Antisense;
TTCCACCCTCTAGAAGTATTTTCTT
>HG-U133A: 213049_at; 611; 61; 1727; Antisense;
AGATAAGCTGCTCACATTTTGTTTT
>HG-U133A: 213049_at; 533; 439; 1747; Antisense;
GTTTTGAATGGGCATCTCCTGAGGA
>HG-U133A: 213049_at; 124; 27; 1760; Antisense;
ATCTCCTGAGGAAATGTAGCATGAC
>HG-U133A: 213049_at; 402; 77; 1777; Antisense;
AGCATGACATTGGTACTAACTGCAT
>HG-U133A: 213049_at; 366; 507; 1788; Antisense;
GGTACTAACTGCATGTGTAAATACA
>HG-U133A: 213049_at; 157; 153; 1807; Antisense;
AATACATCATACTGGCAAACCGTAA
>HG-U133A: 213049_at; 318; 447; 1844; Antisense;
GTATCATCATTCATGTAGTATCTAT
>HG-U133A: 214085_x_at; 260; 369; 138; Antisense;
GAAATTTCCTAACTCTATCAGATAA
>HG-U133A: 214085_x_at; 256; 13; 189; Antisense;
ATTTGCAGGTTGCCACAGGTGGACT
>HG-U133A: 214085_x_at; 451; 455; 222; Antisense;
GTAACCTAACCCATGTTTCAGCTTC
>HG-U133A: 214085_x_at; 357; 57; 297; Antisense;
AGTAACTCCAGTAGCCTTCATTAGT
>HG-U133A: 214085_x_at; 223; 325; 342; Antisense;
GCATGCTGCTTCGACTCTAAATATC
>HG-U133A: 214085_x_at; 461; 649; 359; Antisense;
TAAATATCTGGTTTTCCCTGTCTTT
>HG-U133A: 214085_x_at; 278; 675; 388; Antisense;
TTTACTACTTCCCCAGATTCAGAAC
>HG-U133A: 214085_x_at; 563; 499; 426; Antisense;
GGGGATCTGATTTTAGAGGCCTTAA
>HG-U133A: 214085_x_at; 8; 41; 442; Antisense;
AGGCCTTAATTTTCTGTTCATGGAC
>HG-U133A: 214085_x_at; 313; 29; 543; Antisense;
ATGCTGGGACATCATTACTAACCAA
>HG-U133A: 214085_x_at; 613; 361; 685; Antisense;
GAACACTCTTCTATGAACAACCACC
>HG-U133A: 206662_at; 384; 43; 1035; Antisense;
AGGCTGTGGTCATGCGGAACACTCT
>HG-U133A: 206662_at; 235; 543; 1072; Antisense;
GGCTATCCAGATAATCCTGAACACT
>HG-U133A: 206662_at; 590; 185; 1166; Antisense;
CAGCCCCCTACACCAAGAGTGTATC
>HG-U133A: 206662_at; 60; 367; 1194; Antisense;
GAAAGAGCTCCTACACTTTGAAAAC
>HG-U133A: 206662_at; 297; 255; 1227; Antisense;
CCCTTATCATGAAGTTTGCCTGTTC
>HG-U133A: 206662_at; 119; 163; 1270; Antisense;
AATTTCCTTCAATCTCTAGTGACAA
>HG-U133A: 206662_at; 364; 653; 741; Antisense;
TACTGCCCATTAGCTAAAATCATTT
>HG-U133A: 206662_at; 316; 695; 820; Antisense;
TTCTTTCTAACTACATGCATCTCTC
>HG-U133A: 206662_at; 406; 281; 869; Antisense;
CCACCTTGAAAATCGCTGCTCTGAA
>HG-U133A: 206662_at; 96; 117; 878; Antisense;
AAATCGCTGCTCTGAACCAGTGTTC
>HG-U133A: 206662_at; 632; 709; 996; Antisense;
TTGGTCTTGGTGTCATATGGATCAG
>HG-U133A: 209276_s_at; 504; 169; 103; Antisense;
CAAGAGATCCTCAGTCAATTGCCCA
>HG-U133A: 209276_s_at; 32; 491; 136; Antisense;
GGGCTTCTGGAATTTGTCGATATCA
>HG-U133A: 209276_s_at; 589; 11; 147; Antisense;
ATTTGTCGATATCACAGCCACCAAC
>HG-U133A: 209276_s_at; 484; 281; 164; Antisense;
CCACCAACCACACTAACGAGATTCA
>HG-U133A: 209276_s_at; 56; 427; 190; Antisense;
GATTATTTGCAACAGCTCACGGGAG
>HG-U133A: 209276_s_at; 504; 165; 215; Antisense;
CAAGAACGGTGCCTCGAGTCTTTAT
>HG-U133A: 209276_s_at; 64; 195; 264; Antisense;
CAGTGATCTAGTCTCTTTGCAACAG
>HG-U133A: 209276_s_at; 337; 107; 285; Antisense;
ACAGAGTGGGGAACTGCTGACGCGG
>HG-U133A: 209276_s_at; 185; 567; 30; Antisense;
TGTGAACTGCAAAATCCAGCCTGGG
>HG-U133A: 209276_s_at; 188; 349; 54; Antisense;
GAAGGTGGTTGTGTTCATCAAGCCC
>HG-U133A: 209276_s_at; 402; 283; 77; Antisense;
CCACCTGCCCGTACTGCAGGAGGGC
>HG-U133A: 211284_s_at; 31; 601; 1301; Antisense;
TGCCAGACCCACAAGCCTTGAAGAG
>HG-U133A: 211284_s_at; 399; 397; 1323; Antisense;
GAGAGATGTCCCCTGTGATAATGTC
>HG-U133A: 211284_s_at; 390; 577; 1338; Antisense;
TGATAATGTCAGCAGCTGTCCCTCC
>HG-U133A: 211284_s_at; 703; 297; 1376; Antisense;
GCCGAGACAACCGACAGGGCTGGGC
>HG-U133A: 211284_s_at; 135; 249; 1470; Antisense;
CGCAGCCAGGGGTACCAAGTGTTTG
>HG-U133A: 211284_s_at; 339; 657; 1482; Antisense;
TACCAAGTGTTTGCGCAGGGAGGCC
>HG-U133A: 211284_s_at; 615; 383; 1531; Antisense;
GACCCAGCCTTGAGACAGCTGCTGT
>HG-U133A: 211284_s_at; 185; 405; 1556; Antisense;
GAGGGACAGTACTGAAGACTCTGCA
>HG-U133A: 211284_s_at; 502; 559; 1690; Antisense;
TGGGGCCTCAATCTAAGGCCTTCCC
>HG-U133A: 211284_s_at; 148; 123; 1736; Antisense;
AAAGCCACATTACAAGCTGCCATCC
>HG-U133A: 211284_s_at; 115; 679; 1795; Antisense;
TTTTCCCTATCCACAGGGGTGTTTG
>HG-U133A: 216041_x_at; 190; 395; 1225; Antisense;
GAGAAAGAAGTGGTCTCTGCCCAGC
>HG-U133A: 216041_x_at; 662; 245; 1267; Antisense;
CGTAGCCCTCACGTGGGTGTGAAGG
>HG-U133A: 216041_x_at; 608; 347; 1306; Antisense;
GAAGGACACTTCTGCCATGATAACC
>HG-U133A: 216041_x_at; 652; 599; 1318; Antisense;
TGCCATGATAACCAGACCTGCTGCC
>HG-U133A: 216041_x_at; 702; 297; 1340; Antisense;
GCCGAGACAACCGACAGGGCTGGGC
>HG-U133A: 216041_x_at; 439; 287; 1438; Antisense;
GCCAGGGGTACCAAGTGTTTGCGCA
>HG-U133A: 216041_x_at; 614; 383; 1495; Antisense;
GACCCAGCCTTGAGACAGCTGCTGT
>HG-U133A: 216041_x_at; 540; 193; 1526; Antisense;
CAGTACTGAAGACTCTGCAGCCCTC
>HG-U133A: 216041_x_at; 248; 581; 1630; Antisense;
TGAGCTCCCCATCACCATGGGAGGT
>HG-U133A: 216041_x_at; 501; 559; 1654; Antisense;
TGGGGCCTCAATCTAAGGCCTTCCC
>HG-U133A: 216041_x_at; 147; 123; 1700; Antisense;
AAAGCCACATTACAAGCTGCCATCC
>HG-U133A: 212293_at; 423; 423; 5238; Antisense;
GATAGATGGTGCAGCATGTCTACAT
>HG-U133A: 212293_at; 369; 325; 5251; Antisense;
GCATGTCTACATGGTTGTTTGTTGC
>HG-U133A: 212293_at; 336; 641; 5287; Antisense;
TAATGTGTGGTTTCAATTCAGCTTG
>HG-U133A: 212293_at; 366; 361; 5311; Antisense;
GAAAAATAATCTCACTACATGTAGC
>HG-U133A: 212293_at; 198; 13; 5370; Antisense;
ATTTCTGCTTTGAATCCTTGATATT
>HG-U133A: 212293_at; 514; 5; 5392; Antisense;
ATTGCAATGGAATTCCTACTTTATT
>HG-U133A: 212293_at; 47; 667; 5429; Antisense;
TATGCTAGTTATTGTGTGCGATTTA
>HG-U133A: 212293_at; 45; 683; 5477; Antisense;
TTTTGGTTGTGCGCTTTCTTTTACA
>HG-U133A: 212293_at; 87; 671; 5496; Antisense;
TTTACAACAAGCCTCTAGAAACAGA
>HG-U133A: 212293_at; 384; 443; 5523; Antisense;
GTTTCTGAGAATTACTGAGCTATGT
>HG-U133A: 212293_at; 278; 515; 5669; Antisense;
GGATTCAATGTTTGTCTTTGGTTTT
>HG-U133A: 209657_s_at; 138; 691; 1881; Antisense;
TTCAGGTGTTACTCAGCTGCATAGT
>HG-U133A: 209657_s_at; 247; 479; 1886; Antisense;
GTGTTACTCAGCTGCATAGTTACGC
>HG-U133A: 209657_s_at; 281; 59; 1903; Antisense;
AGTTACGCAGATGTAATGCACATTA
>HG-U133A: 209657_s_at; 112; 705; 1928; Antisense;
TTGGCGTATCTTTAAGTTGGATTCA
>HG-U133A: 209657_s_at; 220; 687; 1939; Antisense;
TTAAGTTGGATTCAAATGGCCATTT
>HG-U133A: 209657_s_at; 55; 419; 2048; Antisense;
GATGCTGTCTATTTGCATTGAGTGT
>HG-U133A: 209657_s_at; 394; 321; 2062; Antisense;
GCATTGAGTGTAAGTCATTTGAACT
>HG-U133A: 209657_s_at; 704; 135; 2159; Antisense;
AACTGGGAACATAAAGTGCCTGTAT
>HG-U133A: 209657_s_at; 207; 175; 2266; Antisense;
CAAAGTGTACGTGAATGCTCGCTGT
>HG-U133A: 209657_s_at; 23; 51; 2296; Antisense;
AGGGTTCCAGCTCCATATATATAGA
>HG-U133A: 209657_s_at; 453; 385; 2348; Antisense;
GAGCCCCATCCAGTTAGTTGGACTA
>HG-U133A;203023_at; 564; 493; 454; Antisense;
GGGAGGACTATAAGGCCATGGCCCG
>HG-U133A: 203023_at; 135; 643; 464; Antisense;
TAAGGCCATGGCCCGTGATGAGAAG
>HG-U133A: 203023_at; 72; 551; 467; Antisense;
GGCCATGGCCCGTGATGAGAAGAAT
>HG-U133A: 203023_at; 366; 539; 473; Antisense;
GGCCCGTGATGAGAAGAATTACTAT
>HG-U133A: 203023_at; 655; 341; 488; Antisense;
GAATTACTATCAAGATACCCCAAAA
>HG-U133A: 203023_at; 226; 367; 501; Antisense;
GATACCCCAAAACAGATTCGGAGTA
>HG-U133A: 203023_at; 580; 613; 518; Antisense;
TCGGAGTAAGATCAACGTCTATAAA
>HG-U133A: 203023_at; 221; 421; 527; Antisense;
GATCAACGTCTATAAACGCTTTTAC
>HG-U133A: 203023_at; 689; 433; 534; Antisense;
GTCTATAAACGCTTTTACCCAGCAG
>HG-U133A: 203023_at; 388; 137; 541; Antisense;
AACGCTTTTACCCAGCAGAGTGGCA
>HG-U133A: 203023_at; 289; 679; 547; Antisense;
TTTACCCAGCAGAGTGGCAAGACTT
>HG-U133A: 210904_s_at; 302; 611; 511; Antisense;
TCCCGACACTAACTATACTCTCTAC
>HG-U133A: 210904_s_at; 506; 101; 522; Antisense;
ACTATACTCTCTACTATTGGCACAG
>HG-U133A: 210904_s_at; 229; 347; 587; Antisense;
GAAGGCCAATACTTTGGTTGTTCCT
>HG-U133A: 210904_s_at; 537; 101; 597; Antisense;
ACTTTGGTTGTTCCTTTGATCTGAC
>HG-U133A: 210904_s_at; 122; 515; 631; Antisense;
GGATTCCAGTTTTGAACAACACAGT
>HG-U133A: 210904_s_at; 265; 123; 692; Antisense;
AAACCATCCTTCAATATAGTGCCTT
>HG-U133A: 210904_s_at; 556; 161; 704; Antisense;
AATATAGTGCCTTTAACTTCCCGTG
>HG-U133A: 210904_s_at; 18; 161; 717; Antisense;
TAACTTCCCGTGTGAAACCTGATCC
>HG-U133A: 210904_s_at; 521; 137; 732; Antisense;
AACCTGATCCTCCACATATTAAAAA
>HG-U133A: 210904_s_at; 433; 229; 760; Antisense;
CTCCTTCCACAATGATGACCTATAT
>HG-U133A: 210904_s_at; 185; 417; 773; Antisense;
GATGACCTATATGTGCAATGGGAGA
>HG-U133A: 205403_at; 372; 397; 1023; Antisense;
GAGAGGATTTGCACATGGATTTTAA
>HG-U133A: 205403_at; 587; 431; 1052; Antisense;
GTTGTCCATAATACCCTGAGTTTTC
>HG-U133A: 205403_at; 75; 579; 1068; Antisense;
TGAGTTTTCAGACACTACGCACCAC
>HG-U133A: 205403_at; 654; 93; 1084; Antisense;
ACGCACCACAGTCAAGGAAGCCTCC
>HG-U133A: 205403_at; 262; 499; 1166; Antisense;
GGGGGAATATGGATGCACAGACGGT
>HG-U133A: 205403_at; 244; 191; 1183; Antisense;
CAGACGGTGCAAACACAGAACTGGA
>HG-U133A: 205403_at; 667; 413; 1214; Antisense;
GATGGTCTGACTGTGCTATGGCCTC
>HG-U133A: 205403_at; 106; 595; 1227; Antisense;
TGCTATGGCCTCATCATCAAGACTT
>HG-U133A: 205403_at; 423; 21; 1242; Antisense;
ATCAAGACTTTCAATCCTATCCCAA
>HG-U133A: 205403_at; 469; 487; 950; Antisense;
GGGCCACGCCAGGAATATTCAGAAA
>HG-U133A: 205403_at; 597; 393; 980; Antisense;
GAGAACTACATTGAAGTGCCATTGA
>HG-U133A: 211372_s_at; 212; 3; 479; Antisense;
ATCTCATACCCGCAAATTTTAACCT
>HG-U133A: 211372_s_at; 456; 57; 523; Antisense;
AGTATGCCCTGACCTGAGTGAATTC
>HG-U133A: 211372_s_at; 504; 507; 579; Antisense;
GGTACAAGGATTCTCTTCTTTTGGA
>HG-U133A: 211372_s_at; 317; 281; 639; Antisense;
CCACTCACTTACTCGTACACGATGT
>HG-U133A: 211372_s_at; 187; 671; 683; Antisense;
TATTACCGCTGTGTCCTGACATTTG
>HG-U133A: 211372_s_at; 293; 347; 713; Antisense;
GAAGGCCAGCAATACAACATCACTA
>HG-U133A: 211372_s_at; 191; 145; 803; Antisense;
AAGACCATATCAGCTTCTCTGGGGT
>HG-U133A: 211372_s_at; 9; 643; 847; Antisense;
TAAGGTGTTTCTGGGAACCGGCACA
>HG-U133A: 211372_s_at; 572; 109; 869; Antisense;
ACACCCTTAACCACCATGCTGTGGT
>HG-U133A: 211372_s_at; 582; 561; 890; Antisense;
TGGTGGACGGCCAATGACACCCACA
>HG-U133A: 211372_s_at; 620; 33; 903; Antisense;
ATGACACCCACATAGAGAGCGCCTA
>HG-U133A: 203828_s_at; 273; 89; 370; Antisense;
ACCTGGAGACAGTGGCGGCTTATTA
>HG-U133A: 203828_s_at; 596; 541; 386; Antisense;
GGCTTATTATGAGGAGCAGCACCCA
>HG-U133A: 203828_s_at; 365; 145; 437; Antisense;
AAGAGATGGATTACGGTGCCGAGGC
>HG-U133A: 203828_s_at; 593; 653; 448; Antisense;
TACGGTGCCGAGGCAACAGATCCCC
>HG-U133A: 203828_s_at; 296; 27; 467; Antisense;
ATCCCCTGTCCCGGATGTTGAGGAT
>HG-U133A: 203828_s_at; 195; 609; 475; Antisense;
TCCCGGATGTTGAGGATCCCGCAAC
>HG-U133A: 203828_s_at; 601; 257; 492; Antisense;
CCCGCAACCGAGGAGCCTGGGGAGA
>HG-U133A: 203828_s_at; 95; 581; 535; Antisense;
TGAGATGGTTCCAGGCCATGCTGCA
>HG-U133A: 203828_s_at; 666; 231; 665; Antisense;
CTGCTCTCTGTCAGAGCTCTTCATG
>HG-U133A: 203828_s_at; 268; 235; 735; Antisense;
CTGACACCCCAGAAGTGCTCTGAAC
>HG-U133A: 203828_s_at; 464; 33; 773; Antisense;
ATGAAGATACTGACACCACCTTTGC
>HG-U133A: 212195_at; 133; 415; 2989; Antisense;
GATGGGTCGTGTGATGAGATGCATT
>HG-U133A: 212195_at; 191; 399; 3004; Antisense;
GAGATGCATTTAAGGCCGATAGTGA
>HG-U133A: 212195_at; 158; 547; 3017; Antisense;
GGCCGATAGTGATAGATGTTTTTTT
>HG-U133A: 212195_at; 674; 673; 3044; Antisense;
TTTCTTGAACACAGGCTTTGTCTGA
>HG-U133A: 212195_at; 660; 345; 3067; Antisense;
GAATGATGTTCTTTTATCTCTTGAA
>HG-U133A: 212195_at; 306; 643; 3121; Antisense;
TAAGTGCTGTTACATTAATACCATA
>HG-U133A: 212195_at; 129; 623; 3197; Antisense;
TCTCTAGTCTCAATATGTATGTGTA
>HG-U133A: 212195_at; 357; 471; 3245; Antisense;
GTGCAATTTGCTAGTAGGACAATGC
>HG-U133A: 212195_at; 505; 529; 3261; Antisense;
GGACAATGCAGTGACTGACTAGCAT
>HG-U133A: 212195_at; 509; 469; 3332; Antisense;
GTGCAATCCTTTCATGTTCACTTGC
>HG-U133A: 212195_at; 433; 667; 3510; Antisense;
TATTAGCTCTAATCCCTTAAGTAAA
>HG-U133A: 207008_at; 225; 83; 2347; Antisense;
ACCTAACGAAGTATCCTTCAGCCTG
>HG-U133A: 207008_at; 162; 105; 2393; Antisense;
ACATGTTACAACACGGACGAACCTT
>HG-U133A: 207008_at; 695; 525; 2407; Antisense;
GGACGAACCTTGAAAACTTTATGCT
>HG-U133A: 207008_at; 564; 15; 2462; Antisense;
ATAGTTTATGATTCCACCTACATGA
>HG-U133A: 207008_at; 675; 425; 2532; Antisense;
GATTACCAGGGACTGAGGGGAGGGG
>HG-U133A: 207008_at; 437; 499; 2553; Antisense;
GGGGAGCATGGGAAGTGACGGTTTA
>HG-U133A: 207008_at; 621; 153; 2577; Antisense;
AATGGGCACAGGGTTTATGTTTAGG
>HG-U133A: 207008_at; 492; 105; 2626; Antisense;
ACAGTAGTGATAGTTGTACCGCAAT
>HG-U133A: 207008_at; 181; 451; 2641; Antisense;
GTACCGCAATGTGACTTAATGCCAC
>HG-U133A: 207008_at; 207; 85; 2763; Antisense;
ACCAAGGCTGATTAAACCAAGGCTA
>HG-U133A: 207008_at; 679; 645; 2775; Antisense;
TAAACCAAGGCTAGAACCACCTGCC
>HG-U133A: 202531_at; 669; 105; 1451; Antisense;
ACAGGAGTCAGTGTCTGGCTTTTTC
>HG-U133A: 202531_at; 400; 547; 1518; Antisense;
TGGCTCCTAGGGGAACAGACCAGTG
>HG-U133A: 202531_at; 163; 645; 1557; Antisense;
TAACACCAATCCCAGGGCTGGCTCT
>HG-U133A: 202531_at; 46; 547; 1572; Antisense;
GGCTGGCTCTGCACTAAGCGAAAAT
>HG-U133A: 202531_at; 546; 601; 1616; Antisense;
TCCAAAGAACTACCCCTTTTCAGCT
>HG-U133A: 202531_at; 62; 257; 1644; Antisense;
CCCTGGGGACTGTTCCAAAGCCAGT
>HG-U133A: 202531_at; 399; 133; 1766; Antisense;
AACTTGGCACTTTTTCGTGTGGATC
>HG-U133A: 202531_at; 258; 485; 1784; Antisense;
GTGGATCTTGCCACATTTCTGATCA
>HG-U133A: 202531_at; 366; 191; 1807; Antisense;
CAGAGGTGTACACTAACATTTCCCC
>HG-U133A: 202531_at; 37; 683; 1852; Antisense;
TTATTTATACAGTGCCTTGCTCGGG
>HG-U133A: 202531_at; 570; 481; 1928; Antisense;
GTGTGAGCGCCTTGGTATGACTTAA
>HG-U133A: 216944_s_at; 217; 335; 8510; Antisense;
GCACTTGAACCAGATTATAGATTTA
>HG-U133A: 216944_s_at; 671; 123; 8563; Antisense;
AAACTAGAATAGCCAGTATTTATGT
>HG-U133A: 216944_s_at; 311; 473; 8601; Antisense;
GTGCAATACGAATTATGCAATCACA
>HG-U133A: 216944_s_at; 440; 21; 8620; Antisense;
ATCACAATACATTTGTAGCTCCCGA
>HG-U133A: 216944_s_at; 473; 159; 8625; Antisense;
AATACATTTGTAGCTCCCGAGTGTC
>HG-U133A: 216944_s_at; 465; 13; 8630; Antisense;
ATTTGTAGCTCCCGAGTGTCCTAAA
>HG-U133A: 216944_s_at; 346; 431; 8634; Antisense;
GTAGCTCCCGAGTGTCCTAAAGGGA
>HG-U133A: 216944_s_at; 50; 467; 8647; Antisense;
GTCCTAAAGGGAGTGCACTTCTTTG
>HG-U133A: 216944_s_at; 484; 101; 8663; Antisense;
ACTTCTTTGAAGCTGGTGTGTTAAT
>HG-U133A: 216944_s_at; 576; 355; 8671; Antisense;
GAAGCTGGTGTGTTAATACTATGTA
>HG-U133A: 216944_s_at; 377; 645; 8705; Antisense;
TAACTTTCAAATGATGCTGCTGCCA
>HG-U133A: 209099_x_at; 346; 673; 5053; Antisense;
TTTGTTTTTCTGCTTTAGACTTGAA
>HG-U133A: 209099_x_at; 581; 395; 5080; Antisense;
GAGACAGGCAGGTGATCTGCTGCAG
>HG-U133A: 209099_x_at; 464; 533; 5198; Antisense;
GGAAGCACACCAATCTGACTTTGTA
>HG-U133A: 209099_x_at; 517; 425; 5229; Antisense;
GATTTCTTTTCACCATTCGTACATA
>HG-U133A: 209099_x_at; 131; 357; 5259; Antisense;
GAACCACTTGTAGATTTGATTTTTT
>HG-U133A: 209099_x_at; 73; 61; 5354; Antisense;
AGATCACTGTTTAGATTTGCCATAG
>HG-U133A: 209099_x_at; 435; 675; 5369; Antisense;
TTTGCCATAGAGTACACTGCCTGCC
>HG-U133A: 209099_x_at; 143; 455; 5380; Antisense;
GTACACTGCCTGCCTTAAGTGAGGA
>HG-U133A: 209099_x_at; 418; 63; 5452; Antisense;
AGAGTAATCTTGTTGGTTCACCATT
>HG-U133A: 209099_x_at; 399; 423; 5488; Antisense;
GATACTTTGTATTGTCCTATTAGTG
>HG-U133A: 209099_x_at; 6; 325; 5531; Antisense;
GCATCTTTGATGTGTTGTTCTTGGC
>HG-U133A: 216268_s_at; 518; 425; 1232; Antisense;
GATTTCTTTTCACCATTCGTACATA
>HG-U133A: 216268_s_at; 331; 161; 1256; Antisense;
AATACTGAACCACTTGTAGATTTGA
>HG-U133A: 216268_s_at; 328; 137; 1321; Antisense;
AAGCTAGTTGAATACTTGAACCATA
>HG-U133A: 216268_s_at; 436; 675; 1373; Antisense;
TTTGCCATAGAGTACACTGCCTGCC
>HG-U133A: 216268_s_at; 144; 455; 1384; Antisense;
GTACACTGCCTGCCTTAAGTGAGGA
>HG-U133A: 216268_s_at; 382; 21; 1410; Antisense;
ATCAAAGTGCTATTACGAAGTTCAA
>HG-U133A: 216268_s_at; 478; 105; 1454; Antisense;
ACAGAGTAATCTTGTTGGTTCACCA
>HG-U133A: 216268_s_at; 475; 573; 1466; Antisense;
TGTTGGTTCACCATTGAGACCGTGA
>HG-U133A: 216268_s_at; 618; 395; 1481; Antisense;
GAGACCGTGAAGATACTTTGTATTG
>HG-U133A: 216268_s_at; 400; 423; 1492; Antisense;
GATACTTTGTATTGTCCTATTAGTG
>HG-U133A: 216268_s_at; 7; 325; 1535; Antisense;
GCATCTTTGATGTGTTGTTCTTGGC
>HG-U133A: 212779_at; 99; 457; 5812; Antisense;
GTCAGTTCTTATCAAAAAGCTCGGT
>HG-U133A: 212779_at; 442; 121; 5827; Antisense;
AAAGCTCGGTACTGCACTACAGGAT
>HG-U133A: 212779_at; 360; 465; 5926; Antisense;
GTCTGTTTTATTACACTGGAGTGTT
>HG-U133A: 212779_at; 103; 1; 6019; Antisense;
GTAAGTTAACCTGTTCTAGTTCCAT
>HG-U133A: 212779_at; 320; 237; 6029; Antisense;
CTGTTCTAGTTCCATCATTCTGTGT
>HG-U133A: 212779_at; 149; 39; 6139; Antisense;
ATGTGCAATACAATTCCTGCATCTT
>HG-U133A: 212779_at; 90; 165; 6150; Antisense;
AATTCCTGCATCTTTAAAATGTCTG
>HG-U133A: 212779_at; 118; 671; 6199; Antisense;
TATTGGATTGGCCGTAACTTTTAGA
>HG-U133A: 212779_at; 168; 495; 6255; Antisense;
GGGAGGTCATTAATTGCTTTTTCTT
>HG-U133A: 212779_at; 483; 87; 6304; Antisense;
ACCTGTTTGTATATAGCTTGAGTAA
>HG-U133A: 212779_at; 451; 3; 6328; Antisense;
ATTGTGATATGATTGTATACCACTA
>HG-U133A: 203543_s_at; 611; 485; 4283; Antisense;
GTGGCTTTTGTCAAGCACTTAGATG
>HG-U133A: 203543_s_at; 627; 61; 4303; Antisense;
AGATGGATATAAATGCAGCAACTTG
>HG-U133A: 203543_s_at; 198; 567; 4425; Antisense;
TGTAACGTATAAACTCAAGCCTTTT
>HG-U133A: 203543_s_at; 311; 153; 4478; Antisense;
AATGTCACAAAACAGGAACCAGCAT
>HG-U133A: 203543_s_at; 217; 419; 4526; Antisense;
GATATGGTTCAAATAGGACTACTAG
>HG-U133A: 203543_s_at; 261; 45; 4540; Antisense;
AGGACTACTAGAGTTCATTGAACAC
>HG-U133A: 203543_s_at; 328; 367; 4677; Antisense;
GAAAGACTATTGCAGGTGTTTAAAA
>HG-U133A: 203543_s_at; 520; 455; 4730; Antisense;
GTAAGTAGTTGTCATATTCTGGAAA
>HG-U133A: 203543_s_at; 608; 687; 4766; Antisense;
TTAGAGTTAAGATATCTCCTCTCTT
>HG-U133A: 203543_s_at; 515; 13; 4777; Antisense;
ATATCTCCTCTCTTTGGTTAGGGAA
>HG-U133A: 203543_s_at; 149; 89; 4816; Antisense;
ACCATTGTGGAATGATGCCCTGGCT
>HG-U133A: 203041_s_at; 652; 57; 1186; Antisense;
AGTATTCTACAGCTCAAGACTGCAG
>HG-U133A: 203041_s_at; 125; 381; 1203; Antisense;
GACTGCAGTGCAGATGACGACAACT
>HG-U133A: 203041_s_at; 104; 329; 1207; Antisense;
GCAGTGCAGATGACGACAACTTCCT
>HG-U133A: 203041_s_at; 294; 473; 1210; Antisense;
GTGCAGATGACGACAACTTCCTTGT
>HG-U133A: 203041_s_at; 9; 193; 1213; Antisense;
CAGATGACGACAACTTCCTTGTGCC
>HG-U133A: 203041_s_at; 506; 209; 1226; Antisense;
CTTCCTTGTGCCCATAGCGGTGGGA
>HG-U133A: 203041_s_at; 584; 567; 1232; Antisense;
TGTGCCCATAGCGGTGGGAGCTGCC
>HG-U133A: 203041_s_at; 333; 311; 1251; Antisense;
GCTGCCTTGGCAGGAGTACTTATTC
>HG-U133A: 203041_s_at; 322; 539; 1259; Antisense;
GGCAGGAGTACTTATTCTAGTGTTG
>HG-U133A: 203041_s_at; 261 625; 1274; Antisense;
TCTAGTGTTGCTGGCTTATTTTATT
>HG-U133A: 203041_s_at; 707; 671; 1295; Antisense;
TATTGGTCTCAAGCACCATCATGCT
>HG-U133A: 212531_at; 179; 169; 400; Antisense;
CAAGAGCTACAATGTCACCTCCGTC
>HG-U133A: 212531_at; 44; 197; 457; Antisense;
CAGGACTTTTGTTCCAGGTTGCCAG
>HG-U133A: 212531_at; 25; 245; 487; Antisense;
CGAGTTCACGCTGGGCAACATTAAG
>HG-U133A: 212531_at; 68; 439; 531; Antisense;
GTTACCTCGTCCGAGTGGTGAGCAC
>HG-U133A: 212531_at; 563; 493; 606; Antisense;
GGGAGTACTTCAAGATCACCCTCTA
>HG-U133A: 212531_at; 114; 61; 618; Antisense;
AGATCACCCTCTACGGGAGAACCAA
>HG-U133A: 212531_at; 616; 493; 632; Antisense;
GGGAGAACCAAGGAGCTGACTTCGG
>HG-U133A: 212531_at; 588; 217; 659; Antisense;
CTAAAGGAGAACTTCATCCGCTTCT
>HG-U133A: 212531_at; 103; 467; 725; Antisense;
GTCCCAATCGACCAGTGTATCGACG
>HG-U133A: 212531_at; 194; 381; 746; Antisense;
GACGGCTGAGTGCACAGGTGCCGCC
>HG-U133A: 212531_at; 396; 181; 779; Antisense;
CACCAGCCCGAACACCATTGAGGGA
>HG-U133A: 202068_s_at; 250; 269; 4602; Antisense;
CCTCCAGTCTGGATCGTTTGACGGG
>HG-U133A: 202068_s_at; 670; 673; 4618; Antisense;
TTTGACGGGACTTCAGGTTCTTTCT
>HG-U133A: 202068_s_at; 474; 369; 4643; Antisense;
GAAATCGCCGTGTTACTGTTGCACT
>HG-U133A: 202068_s_at; 451; 229; 4658; Antisense;
CTGTTGCACTGATGTCCGGAGAGAC
>HG-U133A: 202068_s_at; 487; 459; 4696; Antisense;
GTCAGACTCCCGCGTGAAGATGTCA
>HG-U133A: 202068_s_at; 280; 49; 4773; Antisense;
AGGGAACCGTGATAAGCCTTTCTGG
>HG-U133A: 202068_s_at; 610; 293; 4788; Antisense;
GCCTTTCTGGTTTCGGAGCACGTAA
>HG-U133A: 202068_s_at; 186; 179; 4806; Antisense;
CACGTAAATGCGTCCCTGTACAGAT
>HG-U133A: 202068_s_at; 689; 701; 4951; Antisense;
TTGTTCAGTGACTATTCTCGGGGCC
>HG-U133A: 202068_s_at; 187; 249; 5035; Antisense;
CGAACTGGACTGTGTGCAACGCTTT
>HG-U133A: 202068_s_at; 216; 73; 5065; Antisense;
AGAATGATGTCCCCGTTGTATGTAT
>HG-U133A: 210784_x_at; 632; 527; 1524; Antisense;
GGACAGGGGCCTGCTGAGGAGGTCC
>HG-U133A: 210784_x_at; 523; 363; 1573; Antisense;
GAAAACCTCTATGCTGCCGTGAAGG
>HG-U133A: 210784_x_at; 125; 557; 1619; Antisense;
TGGAGCTGGACAGTCAGAGCCCACA
>HG-U133A: 210784_x_at; 60; 613; 1716; Antisense;
TCCCTCCTCACTGTCTGGGGAATTC
>HG-U133A: 210784_x_at; 289; 413; 1779; Antisense;
GATGGACACTGAGGCTGCTGCATCT
>HG-U133A: 210784_x_at; 105; 27; 1800; Antisense;
ATCTGAAGCCTCCCAGGATGTGACC
>HG-U133A: 210784_x_at; 435; 67; 1852; Antisense;
AGACGGAAGGCAACTGAGCCTCCTC
>HG-U133A: 210784_x_at; 361; 275; 1881; Antisense;
CCAGGAAGGGGAACCTCCAGCTGAG
>HG-U133A: 210784_x_at; 113; 385; 1954; Antisense;
GACCCCACACTCAGCAGAAGGAGAC
>HG-U133A: 210784_x_at; 232; 527; 1982; Antisense;
GGACTGCTGAAGGCACGGGAGCTGC
>HG-U133A: 210784_x_at; 181; 525; 2040; Antisense;
GGACCCCTAACACAGACCATGAGGA
>HG-U133A: 202018_s_at; 510; 249; 2081; Antisense;
CGCTGCTGTGCCTCGATGGCAAACG
>HG-U133A: 202018_s_at; 408; 343; 2151; Antisense;
GAATCATGCCGTGGTGTCTCGGATG
>HG-U133A: 202018_s_at; 1; 71; 2230; Antisense;
AGAAATGGATCTGACTGCCCGGACA
>HG-U133A: 202018_s_at; 624; 379; 2242; Antisense;
GACTGCCCGGACAAGTTTTGCTTAT
>HG-U133A: 202018_s_at; 492; 167; 2253; Antisense;
CAAGTTTTGCTTATTCCAGTCTGAA
>HG-U133A: 202018_s_at; 226; 123; 2283; Antisense;
AAACCTTCTGTTCAATGACAACACT
>HG-U133A: 202018_s_at; 649; 479; 2310; Antisense;
GTGTCTGGCCAGACTCCATGGCAAA
>HG-U133A: 202018_s_at; 27; 493; 2355; Antisense;
GGGACCACAGTATGTCGCAGGCATT
>HG-U133A: 202018_s_at; 661; 605; 2411; Antisense;
TCCTGGAAGCCTGTGAATTCCTCAG
>HG-U133A: 202018_s_at; 186; 169; 2468; Antisense;
CAAGAAAGCCTCAGCCATTCACTGC
>HG-U133A: 202018_s_at; 263; 701; 2572; Antisense;
TTCCCTGCTGTCGTCTTAGCAAGAA
>HG-U133A: 202626_s_at; 119; 385; 1859; Antisense;
GACCCGTCCATTTGGCAGGGGTGGC
>HG-U133A: 202626_s_at; 221; 195; 1874; Antisense;
CAGGGGTGGCTGCCTCATTTAGAGA
>HG-U133A: 202626_s_at; 113; 181; 1913; Antisense;
CACTGGTTGCACTTATGATTTCATG
>HG-U133A: 202626_s_at; 399; 631; 1933; Antisense;
TCATGTGCGGGGATCATCTGCCGTG
>HG-U133A: 202626_s_at; 553; 571; 2021; Antisense;
TGTACTCTTAGATGGATTCTCCACT
>HG-U133A: 202626_s_at; 153; 429; 2035; Antisense;
GATTCTCCACTCAGTTGCAACTTGG
>HG-U133A: 202626_s_at; 87; 339; 2051; Antisense;
GCAACTTGGACTTGTCCTCAGCAGC
>HG-U133A: 202626_s_at; 151; 221; 2067; Antisense;
CTCAGCAGCTGGTAATCTTGCTCTG
>HG-U133A: 202626_s_at; 533; 627; 2082; Antisense;
TCTTGCTCTGCTTGACAACATCTGA
>HG-U133A: 202626_s_at; 381; 127; 2144; Antisense;
AAAATGCACCCAACTAGCTCTATGT
>HG-U133A: 202626_s_at; 471; 395; 2199; Antisense;
GAGACCATTGCAATGAATCCCCAAT
>HG-U133A: 210754_s_at; 175; 223; 1003; Antisense;
CTCATTGACTTTTCTGCTCAGATTG
>HG-U133A: 210754_s_at; 662; 391; 1079; Antisense;
GAGCAGCTAATGTTCTGGTCTCCGA
>HG-U133A: 210754_s_at; 423; 435; 1185; Antisense;
GTTCCCTATTAAGTGGACGGCTCCA
>HG-U133A: 210754_s_at; 86; 469; 1254; Antisense;
GTCCTTTGGAATCCTCCTATACGAA
>HG-U133A: 210754_s_at; 467; 133; 1314; Antisense;
AACTAATGCCGACGTGATGACCGCC
>HG-U133A: 210754_s_at; 134; 237; 1339; Antisense;
CTGTCCCAGGGCTACAGGATGCCCC
>HG-U133A: 210754_s_at; 151; 197; 1353; Antisense;
CAGGATGCCCCGTGTGGAGAACTGC
>HG-U133A: 210754_s_at; 361; 97; 1373; Antisense;
ACTGCCCAGATGAGCTCTATGACAT
>HG-U133A: 210754_s_at; 417; 387; 1433; Antisense;
GACCAACGTTTGACTACTTACAGAG
>HG-U133A: 210754_s_at; 227; 305; 1457; Antisense;
GCGTCCTGGATGATTTCTACACAGC
>HG-U133A: 210754_s_at; 107; 151; 943; Antisense;
AAGGGCAGTTTGCTGGATTTCCTGA
>HG-U133A: 36711_at; 129; 705; 1561; Antisense;
TTGCACGGATCTAAGTTATTCTCCC
>HG-U133A: 36711_at; 90; 667; 2026; Antisense;
TATTGCCCGGCTCCTAGAATTTATT
>HG-U133A: 36711_at; 203; 679; 2049; Antisense;
TTTATTTCCTGACTTACAGCAAGCG
>HG-U133A: 36711_at; 241; 685; 2050; Antisense;
TTATTTCCTGACTTACAGCAAGCGA
>HG-U133A: 36711_at; 633; 669; 2051; Antisense;
TATTTCCTGACTTACAGCAAGCGAG
>HG-U133A: 36711_at; 707; 673; 2053; Antisense;
TTTCCTGACTTACAGCAAGCGAGTT
>HG-U133A: 36711_at; 48; 609; 2055; Antisense;
TCCTGACTTACAGCAAGCGAGTTAT
>HG-U133A: 36711_at; 663; 267; 2056; Antisense;
CCTGACTTACAGCAAGCGAGTTATC
>HG-U133A: 36711_at; 120; 233; 2057; Antisense;
CTGACTTACAGCAAGCGAGTTATCG
>HG-U133A: 36711_at; 401; 583; 2058; Antisense;
TGACTTACAGCAAGCGAGTTATCGT
>HG-U133A: 36711_at; 488; 689; 2062; Antisense;
TTACAGCAAGCGAGTTATCGTCTTC
>HG-U133A: 36711_at; 97; 651; 2063; Antisense;
TACAGCAAGCGAGTTATCGTCTTCT
>HG-U133A: 36711_at; 250; 75; 2066; Antisense;
AGCAAGCGAGTTATCGTCTTCTGTA
>HG-U133A: 36711_at; 594; 169; 2068; Antisense;
CAAGCGAGTTATCGTCTTCTGTATT
>HG-U133A: 36711_at; 108; 141; 2069; Antisense;
AAGCGAGTTATCGTCTTCTGTATTT
>HG-U133A: 36711_at; 149; 305; 2071; Antisense;
GCGAGTTATCGTCTTCTGTATTTTG
>HG-U133A: 207078_at; 510; 391; 522; Antisense;
GAGCAAGGTAAGTAGAACATCCATA
>HG-U133A: 207078_at; 544; 309; 583; Antisense;
GCTGTTTTCTTTAGGAAAATGGCTG
>HG-U133A: 207078_at; 366; 153; 600; Antisense;
AATGGCTGTTGATCTTTTCTAAGTG
>HG-U133A: 207078_at; 637; 575; 609; Antisense;
TGATCTTTTCTAAGTGTGTTTCACT
>HG-U133A: 207078_at; 198; 691; 628; Antisense;
TTCACTTTTTCATGGGATGATGGCT
>HG-U133A: 207078_at; 459; 35; 644; Antisense;
ATGATGGCTTTGTTGCAGCTGAGAT
>HG-U133A: 207078_at; 29; 709; 653; Antisense;
TTGTTGCAGCTGAGATTCATGTAAC
>HG-U133A: 207078_at; 355; 549; 683; Antisense;
TGGTAATAATAGTTTCACATAGGAA
>HG-U133A: 207078_at; 699; 61; 709; Antisense;
AGATGCAAGTTCACTCTGTTAGTTA
>HG-U133A: 207078_at; 268; 179; 720; Antisense;
CACTCTGTTAGTTAACTGGTAGTCT
>HG-U133A: 207078_at; 608; 509; 737; Antisense;
GGTAGTCTTTGTTAAGGTGATTCAA
>HG-U133A: 203003_at; 621; 259; 2881; Antisense;
CCCCAGTTTTTCTCTAAGATATACA
>HG-U133A: 203003_at; 266; 425; 2898; Antisense;
GATATACAGTGCAATAGCTCCCCAC
>HG-U133A: 203003_at; 284; 57; 2928; Antisense;
AGTTGACGCCAGCCCTGTAAAGCTG
>HG-U133A: 203003_at; 429; 461; 2983; Antisense;
GTCTTCAGTGAGGTGGCTGGGGCGA
>HG-U133A: 203003_at; 410; 277; 3036; Antisense;
CCAGGCCAGAGCTCTTTCATTGGGG
>HG-U133A: 203003_at; 204; 489; 3058; Antisense;
GGGCGAGTGTGGTGAGGGGACGTCC
>HG-U133A: 203003_at; 284; 91; 3102; Antisense;
ACCTGGGGGAGTCAACACTGGGATG
>HG-U133A: 203003_at; 507; 129; 3115; Antisense;
AACACTGGGATGGTCTGTGGGGTGG
>HG-U133A: 203003_at; 309; 505; 3139; Antisense;
GGAGGGCCTACGGATGGGTCCGTAG
>HG-U133A: 203003_at; 205; 647; 3338; Antisense;
TAGCGACCTTTGGAAAACGTTAGCG
>HG-U133A: 203003_at; 378; 93; 3354; Antisense;
ACGTTAGCGGTGTAACAGTCCAGGA
>HG-U133A: 204959_at; 386; 401; 1092; Antisense;
GAGGTCCCAAACAGAATTATCGAAA
>HG-U133A: 204959_at; 149; 71; 1125; Antisense;
AAAACTCCCAAGATCAGTCAACTTT
>HG-U133A: 204959_at; 47; 75; 1153; Antisense;
AGCAAGCATCTGGAACAATGGTGTA
>HG-U133A: 204959_at; 141; 155; 1169; Antisense;
AATGGTGTATGGGTTGTTTATGTTA
>HG-U133A: 204959_at; 441; 47; 1301; Antisense;
AGGAGATAAACTTCGACTCTTCTGC
>HG-U133A: 204959_at; 126; 223; 1317; Antisense;
CTCTTCTGCCTTCAACTGAGAACAG
>HG-U133A: 204959_at; 331; 233; 1332; Antisense;
CTGAGAACAGTTGACCGCAAGCTGA
>HG-U133A: 204959_at; 400; 263; 1346; Antisense;
CCGCAAGCTGAAACTGGTGTGTGGA
>HG-U133A: 204959_at; 446; 503; 1361; Antisense;
GGTGTGTGGAAGTCACAGCTTCATC
>HG-U133A: 204959_at; 614; 155; 1451; Antisense;
AATGCAACAAACAACTTCCGCTTAA
>HG-U133A: 204959_at; 36; 17; 1582; Antisense;
ATAGATTAGTTTGCTTTCTGGAATA
>HG-U133A: 210254_at; 518; 583; 1071; Antisense;
TCCACCTACTCCATTGCTTTATGAG
>HG-U133A: 210254_at; 322; 535; 1106; Antisense;
GGAAGGCGGTATAATCCCTATTCAA
>HG-U133A: 210254_at; 24; 133; 1150; Antisense;
AACTTCTGACCGCCCAGTAGGAAGA
>HG-U133A: 210254_at; 128; 321; 1207; Antisense;
GCTTCTTGACTTTAACATCAGCATT
>HG-U133A: 210254_at; 213; 405; 1300; Antisense;
GAGGGTTAAGGCTCAGGGATTTTAT
>HG-U133A: 210254_at; 404; 587; 1330; Antisense;
TGAACTGCTGGAACTCACACATGCC
>HG-U133A: 210254_at; 364; 301; 1382; Antisense;
GCGAGTCTGAGAGCAAGCCCAAATG
>HG-U133A: 210254_at; 117; 339; 1465; Antisense;
GAATCTGACACATCTGGGTTCAAAT
>HG-U133A: 210254_at; 569; 365; 1493; Antisense;
GAAACTGTCACTTATTACCTGTATG
>HG-U133A: 210254_at; 545; 641; 1535; Antisense;
TAATCTCTCTGATCTATTTTTCCTC
>HG-U133A: 210254_at; 177; 113; 1584; Antisense;
ACAACTACTTTGTCGGTTGCTCTGA
>HG-U133A: 203347_s_at; 336; 423; 2172; Antisense;
GATACTAGCCTTAACATGTACCTGT
>HG-U133A: 203347_s_at; 267; 655; 2177; Antisense;
TAGCCTTAACATGTACCTGTCAATG
>HG-U133A: 203347_s_at; 565; 35; 2187; Antisense;
ATGTACCTGTCAATGTTATGGATAT
>HG-U133A: 203347_s_at; 490; 153; 2304; Antisense;
AATGATTGAAACCCATGCATGGTGT
>HG-U133A: 203347_s_at; 258; 33; 2318; Antisense;
ATGCATGGTGTTAGACAATTTTTCT
>HG-U133A: 203347_s_at; 581; 477; 2368; Antisense;
GTGATTAGTGATTATCAGAGCAAAC
>HG-U133A: 203347_s_at; 92; 393; 2385; Antisense;
GAGCAAACATCATGTAGATAGCACA
>HG-U133A: 203347_s_at; 317; 337; 2454; Antisense;
GCAAACATCATGTAGATAGCACAAG
>HG-U133A: 203347_s_at; 467; 11; 2528; Antisense;
ATTTCAATACCTTTTAGATTTCATA
>HG-U133A: 203347_s_at; 130; 149; 2553; Antisense;
AAGTGCAGTGTATATAATGCCTACT
>HG-U133A: 203347_s_at; 305; 665; 2565; Antisense;
TATAATGCCTACTGAAAGACTGTAA
>HG-U133A: 206877_at; 33; 129; 497; Antisense;
GAAAAGCCGTTCACCAAATCGACCA
>HG-U133A: 206877_at; 433; 615; 515; Antisense;
TCGACCAGCTTCAGCGAGAGCAGCG
>HG-U133A: 206877_at; 611; 419; 579; Antisense;
GATCCGGATGGACAGCATCGGCTCC
>HG-U133A: 206877_at; 109; 249; 619; Antisense;
CGCTCCGACTCCGACAGGGAAGAAA
>HG-U133A: 206877_at; 264; 393; 660; Antisense;
GAGCACGGACTATCTCACAGGTGAT
>HG-U133A: 206877_at; 7; 505; 679; Antisense;
GGTGATCTGGACTGGAGCAGCAGCA
>HG-U133A: 206877_at; 59; 329; 701; Antisense;
GCAGTGTGAGCGACTCTGACGAGCG
>HG-U133A: 206877_at; 450; 77; 730; Antisense;
AGCATGCAGAGCCTCGGCAGTGATG
>HG-U133A: 206877_at; 39; 55; 748; Antisense;
AGTGATGAGGGCTATTCCAGCACCA
>HG-U133A: 206877_at; 136; 457; 800; Antisense;
GTCACAAGGCGTGTCTTGGTCTCTA
>HG-U133A: 206877_at; 673; 133; 903; Antisense;
AACTCCCTTGCACGTAAACTTCAGT
>HG-U133A: 201058_s_at; 615; 323; 536; Antisense;
GCATCCTCAAACATGGCGCCAAGGA
>HG-U133A: 201058_s_at; 656; 269; 540; Antisense;
CCTCAAACATGGCGCCAAGGATAAA
>HG-U133A: 201058_s_at; 558; 611; 691; Antisense;
TCCCAGTTCCCAGTGGAAGAAACAG
>HG-U133A: 201058_s_at; 525; 53; 724; Antisense;
AGTGCGTGCCGAGCTGAGGCAGATG
>HG-U133A: 201058_s_at; 239; 305; 727; Antisense;
GCGTGCCGAGCTGAGGCAGATGTTC
>HG-U133A: 201058_s_at; 39; 597; 730; Antisense;
TGCCGAGCTGAGGCAGATGTTCCCA
>HG-U133A: 201058_s_at; 398; 261; 761; Antisense;
CCCCAGAGCCCTGGGCTATAGTCTC
>HG-U133A: 201058_s_at; 360; 189; 764; Antisense;
CAGAGCCCTGGGCTATAGTCTCTGA
>HG-U133A: 201058_s_at; 98; 251; 959; Antisense;
CCCACACAAATGCAAGCTCACCAAG
>HG-U133A: 201058_s_at; 202; 111; 962; Antisense;
ACACAAATGCAAGCTCACCAAGGTC
>HG-U133A: 201058_s_at; 205; 111; 964; Antisense;
ACAAATGCAAGCTCACCAAGGTCCC
>HG-U133A: 205147_x_at; 538; 435; 1051; Antisense;
GTTCGGCTGCTGTCGGATGAGGACG
>HG-U133A: 205147_x_at; 83; 651; 1162; Antisense;
TACAGGGTCTACAACACGATGCCAT
>HG-U133A: 205147_x_at; 262; 77; 690; Antisense;
AGCAGAGGCTCTATTTGACTTCACT
>HG-U133A: 205147_x_at; 449; 307; 745; Antisense;
GCTGGAGATGTGATCTTCCTCCTCA
>HG-U133A: 205147_x_at; 421; 207; 759; Antisense;
CTTCCTCCTCAGTCGGATCAACAAA
>HG-U133A: 205147_x_at; 623; 223; 827; Antisense;
CTCTCTCCTTCGTGAAGATCCTCAA
>HG-U133A: 205147_x_at; 131; 551; 883; Antisense;
TGGCTGCGTTGCTACTACTACGAAG
>HG-U133A: 205147_x_at; 276; 653; 898; Antisense;
TACTACGAAGACACCATCAGCACCA
>HG-U133A: 205147_x_at; 96; 23; 913; Antisense;
ATCAGCACCATCAAGGACATCGCGG
>HG-U133A: 205147_x_at; 258; 179; 957; Antisense;
CACTCCCCTATTGAAAGACCTGCTG
>HG-U133A: 205147_x_at; 638; 523; 981; Antisense;
GGAGCTCACAAGGCGGGAGTTCCAG
>HG-U133A: 207677_s_at; 696; 557; 1183; Antisense;
TGGAGGAAGATCTCAGCAGCACTCC
>HG-U133A: 207677_s_at; 257; 179; 1202; Antisense;
CACTCCCCTATTGAAAGACCTGCTG
>HG-U133A: 207677_s_at; 637; 523; 1226; Antisense;
GGAGCTCACAAGGCGGGAGTTCCAG
>HG-U133A: 207677_s_at; 252; 527; 1256; Antisense;
GGACATAGCTCTGAATTACCGGGAC
>HG-U133A: 207677_s_at; 43; 313; 1263; Antisense;
GCTCTGAATTACCGGGACGCTGAGG
>HG-U133A: 207677_s_at; 537; 435; 1296; Antisense;
GTTCGGCTGCTGTCGGATGAGGACG
>HG-U133A: 207677_s_at; 119; 417; 1311; Antisense;
GATGAGGACGTAGCGCTCATGGTGC
>HG-U133A: 207677_s_at; 355; 533; 1378; Antisense;
GGAAGCTGCACATCACGCAGAAGGA
>HG-U133A: 207677_s_at; 316; 595; 836; Antisense;
TCGTGAAGATCCTCAAAGACTTCCC
>HG-U133A: 207677_s_at; 130; 551; 883; Antisense;
TGGCTGCGTTGCTACTACTACGAAG
>HG-U133A: 207677_s_at; 484; 593; 892; Antisense;
TGCTACTACTACGAAGACACCATCA
>HG-U133A: 209959_at; 536; 681; 4461; Antisense;
TTTTATTTTTACACCCATCAGATTT
>HG-U133A: 209959_at; 678; 683; 4610; Antisense;
TTATTACAACTATGAGAGCCTCCCA
>HG-U133A: 209959_at; 4; 271; 4628; Antisense;
CCTCCCAAGTCATCTTATCAACTCA
>HG-U133A: 209959_at; 183; 517; 4699; Antisense;
GGATGACCACACTAGCACAGAAGAG
>HG-U133A: 209959_at; 645; 687; 4735; Antisense;
TTAAAGCAGGTGATTCCTCCCTTGG
>HG-U133A: 209959_at; 434; 429; 4746; Antisense;
GATTCCTCCCTTGGCGGGAGAGCTC
>HG-U133A: 209959_at; 72; 391; 4765; Antisense;
GAGCTCTCTCAGTGTGAACATGCCT
>HG-U133A: 209959_at; 678; 199; 4783; Antisense;
CATGCCTTCTGTGGGCGGAAATCAG
>HG-U133A: 209959_at; 587; 529; 4799; Antisense;
GGAAATCAGGAAGCCACCAGCTGTT
>HG-U133A: 209959_at; 575; 519; 4827; Antisense;
GGAGAGTGCCTTGCTTTTATTTCAG
>HG-U133A: 209959_at; 39; 595; 4877; Antisense;
TGCTCCTCTAACAGCATTGCTCTTT
>HG-U133A: 206343_s_at; 3; 63; 1481; Antisense;
AGATCTAATATTGACTGCCTCTGCC
>HG-U133A: 206343_s_at; 499; 269; 1498; Antisense;
CCTCTGCCTGTCGCATGAGAACATT
>HG-U133A: 206343_s_at; 111; 165; 1531; Antisense;
CAATTGTATTACTTCCTCTGTTCGC
>HG-U133A: 206343_s_at; 79; 699; 1551; Antisense;
TTCGCGACTAGTTGGCTCTGAGATA
>HG-U133A: 206343_s_at; 218; 473; 1587; Antisense;
GTGAGGCTCCGGATGTTTCTGGAAT
>HG-U133A: 206343_s_at; 612; 19; 1669; Antisense;
ATAAAGGCATTTCAAAGTCTCACTT
>HG-U133A: 206343_s_at; 623; 623; 1716; Antisense;
TCTACTGAACAGTCCATCTTCTTTA
>HG-U133A: 206343_s_at; 311; 627; 1732; Antisense;
TCTTCTTTATACAATGACCACATCC
>HG-U133A: 206343_s_at; 314; 387; 1747; Antisense;
GACCACATCCTGAAAAGGGTGTTGC
>HG-U133A: 206343_s_at; 209; 51; 1762; Antisense;
AGGGTGTTGCTAAGCTGTAACCGAT
>HG-U133A: 206343_s_at; 146; 79; 1774; Antisense;
AGCTGTAACCGATATGCACTTGAAA
>HG-U133A: 202599_s_at; 563; 11; 6713; Antisense;
ATTTAAGTTGTGATTACCTGCTGCA
>HG-U133A: 202599_s_at; 232; 149; 6742; Antisense;
AAGTGGCATGGGGGACCCTGTGCAT
>HG-U133A: 202599_s_at; 571; 381; 6755; Antisense;
GACCCTGTGCATCTGTGCATTTGGC
>HG-U133A: 202599_s_at; 82; 605; 6829; Antisense;
TCCATTTCTGGACATGACGTCTGTG
>HG-U133A: 202599_s_at; 622; 381; 6844; Antisense;
GACGTCTGTGGTTTAAGCTTTGTGA
>HG-U133A: 202599_s_at; 311; 155; 6872; Antisense;
AATGTGCTTTGATTCGAAGGGTCTT
>HG-U133A: 202599_s_at; 266; 629; 6909; Antisense;
TAATCGTCAACCACTTTTAAACATA
>HG-U133A: 202599_s_at; 339; 73; 6935; Antisense;
AGAATTCACACAACTACTTTCATGA
>HG-U133A: 202599_s_at; 334; 5; 6985; Antisense;
ATTCCAAGAGTATCCCAGTATTAGC
>HG-U133A: 202599_s_at; 140; 15; 7019; Antisense;
ATATAGGCACATTACCATTCATAGT
>HG-U133A: 202599_s_at; 707; 161; 7069; Antisense;
AATTTGATGCGATCTGCTCAGTAAT
>HG-U133A: 207740_s_at; 67; 187; 1282; Antisense;
CAGCCTGCACCGCGAGGTGGAGAAG
>HG-U133A: 207740_s_at; 176; 385; 1331; Antisense;
GACCAGGAGCTCGACTTCATCCTGT
>HG-U133A: 207740_s_at; 194; 625; 1425; Antisense;
TCTACCTGCAGCACGCGGATGAGGA
>HG-U133A: 207740_s_at; 598; 305; 1468; Antisense;
GCTGGCTGAGAACATCGACGCACAG
>HG-U133A: 207740_s_at; 163; 615; 1482; Antisense;
TCGACGCACAGCTCAAGCGCATGGC
>HG-U133A: 207740_s_at; 572; 551; 1503; Antisense;
TGGCCCAGGATCTCAAGGACATCAT
>HG-U133A: 207740_s_at; 8; 153; 1517; Antisense;
AAGGACATCATCGAGCACCTGAACA
>HG-U133A: 207740_s_at; 659; 327; 1618; Antisense;
GCAGTGGATCGACCAGAACTCGGCC
>HG-U133A: 207740_s_at; 129; 423; 1717; Antisense;
GATCACCTTTGACTGAGCGACAGCA
>HG-U133A: 207740_s_at; 603; 33; 1773; Antisense;
ATGAGGGGAATGCGCCCTGTTGTCT
>HG-U133A: 207740_s_at; 298; 431; 1791; Antisense;
GTTGTCTGTAGTTTGGGGTTGTGGC
>HG-U133A: 202900_s_at; 449; 59; 2016; Antisense;
AGTTCTCTCTGATAGTGAGCGAGAC
>HG-U133A: 202900_s_at; 93; 341; 2050; Antisense;
GAATTACAGCTGATACCTGATCAAC
>HG-U133A: 202900_s_at; 320; 423; 2061; Antisense;
GATACCTGATCAACTTCGACATTTG
>HG-U133A: 202900_s_at; 158; 101; 2073; Antisense;
ACTTCGACATTTGGGCAATGCCATC
>HG-U133A: 202900_s_at; 483; 489; 2085; Antisense;
GGGCAATGCCATCAAACAGGTTACT
>HG-U133A: 202900_s_at; 116; 29; 2090; Antisense;
ATGCCATCAAACAGGTTACTATGAA
>HG-U133A: 202900_s_at; 190; 393; 2140; Antisense;
GAGAAGGTGTTGAGTCTTCCAAAAC
>HG-U133A: 202900_s_at; 709; 621; 2175; Antisense;
TCTCAGTGCCTACCAGCGAAAGTGC
>HG-U133A: 202900_s_at; 78; 471; 2180; Antisense;
GTGCCTACCAGCGAAAGTGCATTCA
>HG-U133A: 202900_s_at; 9; 83; 2189; Antisense;
AGCGAAAGTGCATTCAGTCCATCCT
>HG-U133A: 202900_s_at; 187; 367; 2192; Antisense;
GAAAGTGCATTCAGTCCATCCTGAA
>HG-U133A: 209791_at; 397; 229; 3781; Antisense;
CTCCAGCCCCAGAGCTGAAAACACC
>HG-U133A: 209791_at; 21; 273; 3811; Antisense;
CCTATTTGAGGGTGTCTGTCTGGAG
>HG-U133A: 209791_at; 103; 685; 3926; Antisense;
TTAGGGGGAAGTGAGCGCCTCCCAT
>HG-U133A: 209791_at; 521; 641; 3981; Antisense;
TAAGGCTTTCCCCAATGATGTCGGT
>HG-U133A: 209791_at; 442; 415; 3997; Antisense;
GATGTCGGTAATTTCTGATGTTTCT
>HG-U133A: 209791_at; 200; 617; 4019; Antisense;
TCTGAAGTTCCCAGGACTCACACAC
>HG-U133A: 209791_at; 375; 181; 4064; Antisense;
CACCCAGTGTGACAACCCTCGGTGT
>HG-U133A: 209791_at; 685; 369; 4074; Antisense;
GACAACCCTCGGTGTGGATATACCC
>HG-U133A: 209791_at; 57; 283; 4120; Antisense;
CCACCCCCACTTTCTATAAATGTAG
>HG-U133A: 209791_at; 520; 547; 4144; Antisense;
GGCCTAGAATACGCTTCTCTGTTGC
>HG-U133A: 209791_at; 153; 489; 4223; Antisense;
GGGCAGGGGATGTCGTGAAGATGGC
>HG-U133A: 210837_s_at; 641; 373; 2532; Antisense;
GACATAGCACGAATCTGTTACCAGT
>HG-U133A: 210837_s_at; 485; 519; 2561; Antisense;
GGAGGATGAGCCACAGAAATTGCAT
>HG-U133A: 210837_s_at; 241; 639; 2593; Antisense;
TAATTTCAAGTCTTCCTGATACATG
>HG-U133A: 210837_s_at; 164; 343; 2621; Antisense;
GAATAGTGTGGTTCAGTGAGCTGCA
>HG-U133A: 210837_s_at; 215; 233; 2641; Antisense;
CTGCACTGACCTCTACATTTTGTAT
>HG-U133A: 210837_s_at; 167; 217; 2739; Antisense;
CTATGTTCAGAACTTCATCTGCCAC
>HG-U133A: 210837_s_at; 151; 57; 2801; Antisense;
AGTACAAATCTGTGCTACACTGGAT
>HG-U133A: 210837_s_at; 704; 11; 2835; Antisense;
ATTTATGAATTTTACTTGCACCTTA
>HG-U133A: 210837_s_at; 499; 689; 2846; Antisense;
TTACTTGCACCTTATAGTTCATAGC
>HG-U133A: 210837_s_at; 581; 667; 2907; Antisense;
TATACCAATGACTTCCATATTTTAA
>HG-U133A: 210837_s_at; 326; 133; 2942; Antisense;
CAACTTTATGTTGCAGGAAACCCTT
>HG-U133A: 208983_s_at; 656; 401; 1842; Antisense;
GAGTATTACTGCACAGCCTTCAACA
>HG-U133A: 208983_s_at; 215; 629; 1972; Antisense;
TCATTGCTCTCTTGATCATTGCGGC
>HG-U133A: 208983_s_at; 488; 555; 2041; Antisense;
TGGAAATGTCCAGGCCAGCAGTACC
>HG-U133A: 208983_s_at; 671; 655; 2062; Antisense;
TACCACTTCTGAACTCCAACAACGA
>HG-U133A: 208983_s_at; 187; 523; 2108; Antisense;
GGAAGCTAACAGTCATTACGGTCAC
>HG-U133A: 208983_s_at; 208; 389; 2181; Antisense;
GAGCCTCTGAACTCAGACGTGCAGT
>HG-U133A: 208983_s_at; 39; 481; 2220; Antisense;
GTGTCCTCAGCTGAGTCTCACAAAG
>HG-U133A: 208983_s_at; 117; 53; 2277; Antisense;
AGTGAAGTCCGGAAAGCTGTCCCTG
>HG-U133A: 208983_s_at; 588; 235; 2293; Antisense;
CTGTCCCTGATGCCGTGGAAAGCAG
>HG-U133A: 208983_s_at; 486; 343; 2331; Antisense;
GAAGGCTCCCTTGATGGAACTTAGA
>HG-U133A: 208983_s_at; 553; 373; 2354; Antisense;
GACAGCAAGGCCAGATGCACATCCC
>HG-U133A: 205361_s_at; 221; 153; 144; Antisense;
AAGGCGGCTGCAGAAGATGTCAATG
>HG-U133A: 205361_s_at; 233; 61; 158; Antisense;
AGATGTCAATGTTACTTTCGAAGAT
>HG-U133A: 205361_s_at; 442; 123; 269; Antisense;
AAACCTAGAAGATGCTTGTGATGAC
>HG-U133A: 205361_s_at; 458; 705; 284; Antisense;
TTGTGATGACATCATGCTTGCAGAT
>HG-U133A: 205361_s_at; 567; 415; 309; Antisense;
GATGATTGCTTAATGATACCTTATC
>HG-U133A: 205361_s_at; 594; 423; 323; Antisense;
GATACCTTATCAAATTGGTGATGTC
>HG-U133A: 205361_s_at; 160; 505; 339; Antisense;
GGTGATGTCTTCATTAGCCATTCTC
>HG-U133A: 205361_s_at; 253; 697; 418; Antisense;
TTGACGCCTTAGAATCCAGAGTGGA
>HG-U133A: 205361_s_at; 144; 533; 440; Antisense;
GGAATCAATTCAGCGAGTGTTAGCA
>HG-U133A: 205361_s_at; 440; 437; 474; Antisense;
GTTCAGTTGTATGCAAAATTCGGGA
>HG-U133A: 205361_s_at; 36; 493; 495; Antisense;
GGGAGCAACATAAACCTTGAAGCTG
>HG-U133A: 222371_at; 300; 33; 127; Antisense;
ATGATGTTTACCAGTCCATTTCAGT
>HG-U133A: 222371_at; 651; 677; 133; Antisense;
TTTACCAGTCCATTTCAGTTCTTCA
>HG-U133A: 222371_at; 411; 223; 13; Antisense;
CTCTTATAGCCCCACATAGGTTAGA
>HG-U133A: 222371_at; 331; 99; 180; Antisense;
ACTACTTTTGCTGTATACCAAGCTA
>HG-U133A: 222371_at; 204; 457; 309; Antisense;
GTAAGAGACAGAGTGCATTCATTTG
>HG-U133A: 222371_at; 564; 591; 322; Antisense;
TGCATTCATTTGCACCCAGGGTTGG
>HG-U133A: 222371_at; 629; 13; 377; Antisense;
ATATTGGAGATACTTGGCTATTTGT
>HG-U133A: 222371_at; 548; 89; 402; Antisense;
ACCTCACCTGCCCATGAAGGCTAAA
>HG-U133A: 222371_at; 553; 557; 429; Antisense;
TGGATGGTTAAACACCTGTCTCTGT
>HG-U133A: 222371_at; 228; 417; 82; Antisense;
GATGCTTAAGAAATTACCTCACATA
>HG-U133A: 222371_at; 467; 9; 94; Antisense;
ATTACCTCACATAAACATTTTACCA
>HG-U133A: 215236_s_at; 537; 551; 450; Antisense;
TGGACTTCTCAAACCAACAGTGGCC
>HG-U133A: 215236_s_at; 178; 207; 454; Antisense;
CTTCTCAAACCAACAGTGGCCTCTC
>HG-U133A: 215236_s_at; 151; 219; 457; Antisense;
CTCAAACCAACAGTGGCCTCTCAGA
>HG-U133A: 215236_s_at; 512; 129; 465; Antisense;
AACAGTGGCCTCTCAGAACCAGAAC
>HG-U133A: 215236_s_at; 633; 613; 506; Antisense;
TCCCACCTAGCAAGTTAGTATCTGA
>HG-U133A: 215236_s_at; 384; 447; 523; Antisense;
GTATCTGATGACTTGGATTCATCTT
>HG-U133A: 215236_s_at; 292; 377; 532; Antisense;
GACTTGGATTCATCTTTAGCCAACC
>HG-U133A: 215236_s_at; 678; 425; 538; Antisense;
GATTCATCTTTAGCCAACCTTGTGG
>HG-U133A: 215236_s_at; 677; 627; 541; Antisense;
TCATCTTTAGCCAACCTTGTGGGCA
>HG-U133A: 215236_s_at; 649; 665; 547; Antisense;
TTAGCCAACCTTGTGGGCAATCTTG
>HG-U133A: 215236_s_at; 191; 139; 553; Antisense;
AACCTTGTGGGCAATCTTGGCATCG
>HG-U133A: 205281_s_at; 534; 115; 3027; Antisense;
AAATGGACTGATCTTTAAACTATTC
>HG-U133A: 205281_s_at; 65; 135; 3044; Antisense;
AACTATTCAGTCTTACTGGGATTTT
>HG-U133A: 205281_s_at; 619; 19; 3099; Antisense;
ATAAACAGTGCCAGTATTCATAGGA
>HG-U133A: 205281_s_at; 612; 473; 3126; Antisense;
GTGAGAAACTGTAATATTTGGCCAT
>HG-U133A: 205281_s_at; 406; 293; 3146; Antisense;
GCCATTATTCTATTCAACAGGTTTT
>HG-U133A: 205281_s_at; 653; 637; 3159; Antisense;
TCAACAGGTTTTAGAGGCATGCCAC
>HG-U133A: 205281_s_at; 550; 59; 3276; Antisense;
AGTTGCCTTTGCCTGTAAAACATGT
>HG-U133A: 205281_s_at; 249; 667; 3370; Antisense;
TTTCAACCTTTCTGGATACCTTAAT
>HG-U133A: 205281_s_at; 667; 455; 3396; Antisense;
GTAACTGTCAGTTTGCACTGGTCGG
>HG-U133A: 205281_s_at; 598; 589; 3409; Antisense;
TGCACTGGTCGGTATATGGAAACAC
>HG-U133A: 205281_s_at; 580; 663; 3423; Antisense;
TATGGAAACACATTGCTCTACCCTG
>HG-U133A: 203057_s_at; 54; 31; 5545; Antisense;
ATGCCTATTCTGGTGTTGCGTTTGT
>HG-U133A: 203057_s_at; 391; 381; 5581; Antisense;
GACGTTATCCTCTCAGATTCTTATC
>HG-U133A: 203057_s_at; 327; 667; 5651; Antisense;
TATATCAGTGCACAGGCGCATCCCA
>HG-U133A: 203057_s_at; 250; 323; 5668; Antisense;
GCATCCCAGGCCTGTACAGATGTAT
>HG-U133A: 203057_s_at; 516; 655; 5722; Antisense;
TACCAGGTTTTACACTTGCATCTCT
>HG-U133A: 203057_s_at; 46; 165; 5767; Antisense;
AATTGGCCTCTTCCTAAGTATATTA
>HG-U133A: 203057_s_at; 519; 679; 5798; Antisense;
TTTATCCTTACATTTTATGCCTCCC
>HG-U133A: 203057_s_at; 616; 661; 5813; Antisense;
TATGCCTCCCCCTAAATTAATGACT
>HG-U133A: 203057_s_at; 140; 81; 5850; Antisense;
AGCGGCTAGGTTTTATTCATACTGT
>HG-U133A: 203057_s_at; 644; 585; 5938; Antisense;
TGAATTTGTGCCACTTTAATCCTTC
>HG-U133A: 203057_s_at; 465; 703; 5960; Antisense;
TTCCACTATCATTCCCATTTTGTTA
>HG-U133A: 200604_s_at; 443; 595; 1015; Antisense;
TGCTGTGCTACAACGTCGGTCAGAA
>HG-U133A: 200604_s_at; 516; 99; 1099; Antisense;
ACTACTGATGAATCGTCCTCGTGCT
>HG-U133A: 200604_s_at; 439; 587; 1149; Antisense;
TGAAGTGCGTTAAGCTGGACCGACC
>HG-U133A: 200604_s_at; 343; 287; 1164; Antisense;
TGGACCGACCTAGATTTGAACGTGT
>HG-U133A: 200604_s_at; 669; 11; 1177; Antisense;
ATTTGAACGTGTTCTTGGCCCATGC
>HG-U133A: 200604_s_at; 689; 285; 1296; Antisense;
CCAATCCATGCTTCACTCATGCAAA
>HG-U133A: 200604_s_at; 366; 319; 1305; Antisense;
GCTTCACTCATGCAAACTGCTTTAT
>HG-U133A: 200604_s_at; 348; 5; 1403; Antisense;
ATTGCACCATTTTCAATTTGGAGCA
>HG-U133A: 200604_s_at; 704; 43; 897; Antisense;
AGTGGGAACGTCTTACGGTAGCTGA
>HG-U133A: 200604_s_at; 679; 449; 914; Antisense;
GTAGCTGATGCATTGGAACCAGTGC
>HG-U133A: 200604_s_at; 296; 11; 998; Antisense;
ATTTTAGAGGGGTCAGCTGCTGTGC
>HG-U133A: 220553_s_at; 480; 673; 1543; Antisense;
TTTGATAACCTGTCTTCCTTGTTTC
>HG-U133A: 220553_s_at; 126; 457; 1604; Antisense;
GTCAATTAGTAGCTTACCACAGATA
>HG-U133A: 220553_s_at; 447; 177; 1621; Antisense;
CACAGATACTGTTTCCTACCATTTA
>HG-U133A: 220553_s_at; 395; 575; 1678; Antisense;
TGATTTTTGCATTAAGTGGTCTAGA
>HG-U133A: 220553_s_at; 399; 483; 1693; Antisense;
GTGGTCTAGAATTCTTTTGCAATGC
>HG-U133A: 220553_s_at; 108; 673; 1719; Antisense;
TTTGCAACAGAATTTTGTAGCCTTA
>HG-U133A: 220553_s_at; 379; 361; 1755; Antisense;
GAAAAACCTGACTGCAAATCATGTC
>HG-U133A: 220553_s_at; 262; 175; 1803; Antisense;
CACATAAGGGCTGGTTATTTACCTC
>HG-U133A: 220553_s_at; 192; 45; 1855; Antisense;
AGGACTTTTAACCTTTGCTGACAAG
>HG-U133A: 220553_s_at; 191; 705; 1883; Antisense;
TTGTCTGTTTCAGTTATACTTGTGA
>HG-U133A: 220553_s_at; 470; 157; 1943; Antisense;
AATACTTTGCCTTGGAATAGATTAT
>HG-U133A: 200749_at; 156; 577; 2024; Antisense;
TGATCCCATCAACACTATTCTTGTA
>HG-U133A: 200749_at; 640; 201; 2067; Antisense;
CTATTTTTTTCTCATACGATTACTA
>HG-U133A: 200749_at; 240; 95; 2082; Antisense;
ACGATTACTATAGTCCAGTTTACCA
>HG-U133A: 200749_at; 386; 627; 2131; Antisense;
TCTTGAGATGATTGCTTACCTTAAA
>HG-U133A: 200749_at; 16; 437; 2216; Antisense;
GTTCTACTTACTGTATTAACTGGCA
>HG-U133A: 200749_at; 613; 145; 2256; Antisense;
AAGATCTGAATTGCTGTGTATGTTA
>HG-U133A: 200749_at; 420; 437; 2277; Antisense;
GTTACGCTGTATTCAGAACCAGTTT
>HG-U133A: 200749_at; 228; 357; 2292; Antisense;
GAACCAGTTTCTAACCAGCCTGTGA
>HG-U133A: 200749_at; 301; 53; 2414; Antisense;
AGTGGTGTTGACATTCTGGATCTTC
>HG-U133A: 200749_at; 685; 471; 2462; Antisense;
GTGACGTCACTTACCTGTCTAACGT
>HG-U133A: 200749_at; 120; 655; 2473; Antisense;
TACCTGTCTAACGTGGTGTGGGAGA
>HG-U133A: 201713_s_at; 109; 395; 9419; Antisense;
GAGAACTTCAGAGCACTATGCACTG
>HG-U133A: 201713_s_at; 617; 637; 9462; Antisense;
TCAAGAATTCCATTTTTCACAGAGT
>HG-U133A: 201713_s_at; 299; 457; 9485; Antisense;
GTAATTCCAGATTTTGTTTGCCAAG
>HG-U133A: 201713_s_at; 464; 529; 9533; Antisense;
GGAACAGGCGGACAGTCCATTTATG
>HG-U133A: 201713_s_at; 213; 415; 9584; Antisense;
GATGTGAAACATACTGGTCCTGGTT
>HG-U133A: 201713_s_at; 190; 287; 9620; Antisense;
GCCAATCAAGGCCAGAATACCAATA
>HG-U133A: 201713_s_at; 25; 143; 9692; Antisense;
AAGCATGTAGTATTTGGGTTTGTTA
>HG-U133A: 201713_s_at; 170; 61; 9741; Antisense;
AGATTGAATCATTTGGTTCTCCCAA
>HG-U133A: 201713_s_at; 379; 329; 9851; Antisense;
GCAGTTGGATTGAAGCTTAGCTATT
>HG-U133A: 201713_s_at; 615; 521; 9908; Antisense;
TGGACGTTTCCGATTTACAAATGTA
>HG-U133A: 201713_s_at; 302; 331; 9938; Antisense;
GCAGCTTATAGCTGTTGTCACTTTT
>HG-U133A: 209085_x_at; 23; 209; 3920; Antisense;
CTTGTTTTTTCCCAGAGCAACCATG
>HG-U133A: 209085_x_at; 391; 511; 3958; Antisense;
GGATGACCTGGTGTCCCATTATAAA
>HG-U133A: 209085_x_at; 598; 327; 4012; Antisense;
GCAGTAGGCTTATGTACACCTCTTA
>HG-U133A: 209085_x_at; 384; 575; 4044; Antisense;
TGATAGGACTGCTTGGGTCCTCCAC
>HG-U133A: 209085_x_at; 568; 179; 4066; Antisense;
CACTGTCCTCTGTCAATCTAGTTAG
>HG-U133A: 209085_x_at; 548; 435; 4086; Antisense;
GTTAGACGTGCTTCTGAATGACTGT
>HG-U133A: 209085_x_at; 81; 531; 4117; Antisense;
GGAACTAGAAACTACACCTGGCTTG
>HG-U133A: 209085_x_at; 54; 179; 4131; Antisense;
CACCTGGCTTGGAGTCAGATTTAGT
>HG-U133A: 209085_x_at; 595; 327; 4175; Antisense;
GCAGTAGTACTAAGGCGTCTTTTGT
>HG-U133A: 209085_x_at; 666; 71; 4208; Antisense;
AGAATTTATCCTAATGGCCTTTATA
>HG-U133A: 209085_x_at; 600; 55; 4308; Antisense;
AGTCACCACCTAGAACTGGGTATTC
>HG-U133A: 206111_at; 357; 439; 176; Antisense;
GTTTACCTGGGCTCAATGGTTTGAA
>HG-U133A: 206111_at; 653; 365; 198; Antisense;
GAAACCCAGCACATCAATATGACCT
>HG-U133A: 206111_at; 531; 637; 211; Antisense;
TCAATATGACCTCCCAGCAATGCAC
>HG-U133A: 206111_at; 609; 701; 291; Antisense;
TTCCTTCTTACAACTTTTGCTAACG
>HG-U133A: 206111_at; 655; 453; 331; Antisense;
GTAACCCAAATATGACCTGTCCTAG
>HG-U133A: 206111_at; 93; 385; 344; Antisense;
GACCTGTCCTAGTAACAAAACTCGC
>HG-U133A: 206111_at; 490; 133; 362; Antisense;
AACTCGCAAAAATTGTCACCACAGT
>HG-U133A: 206111_at; 674; 139; 389; Antisense;
AAGCCAGGTGCCTTTAATCCACTGT
>HG-U133A: 206111_at; 512; 189; 503; Antisense;
CAGAGATCAACGACGAGACCCTCCA
>HG-U133A: 206111_at; 137; 193; 544; Antisense;
CAGTTCACCTGGATAGAATCATCTA
>HG-U133A: 206111_at; 57; 29; 630; Antisense;
ATCCCATCTCTCCATATACTTTGGG
>HG-U133A: 201779_s_at; 336; 433; 1007; Antisense;
GTTGTTCCTTCTCAAGGCGATTCAG
>HG-U133A: 201779_s_at; 392; 547; 1022; Antisense;
GGCGATTCAGACTCTGACACAGACA
>HG-U133A: 201779_s_at; 171; 473; 1067; Antisense;
GTGACAGAACATACCCCTTTACTGA
>HG-U133A: 201779_s_at; 80; 581; 1089; Antisense;
TGAGACCTTTAGCTTCTGTCAGTGC
>HG-U133A: 201779_s_at; 317; 279; 1114; Antisense;
CCAGTCATTTGGGGCTTTATCGGAA
>HG-U133A: 201779_s_at; 626; 685; 1130; Antisense;
TTATCGGAATCCCGCTCACATCAGA
>HG-U133A: 201779_s_at; 41; 361; 1232; Antisense;
GAACATGATGTCGTGGTCCAGTTGC
>HG-U133A: 201779_s_at; 654; 483; 1244; Antisense;
GTGGTCCAGTTGCAGCCTAATGGTG
>HG-U133A: 201779_s_at; 519; 427; 1359; Antisense;
GATTTTTTGCTCCCTTCAAAGATTT
>HG-U133A: 201779_s_at; 584; 381; 1493; Antisense;
GACTGGTGCTGTAACTCAAGCATCA
>HG-U133A: 201779_s_at; 529; 123; 981; Antisense;
AAACCTGTCCAGTGTGCAAGCAAAA
>HG-U133A: 222204_s_at; 122; 473; 334; Antisense;
GTGCAAACACTGCTAGAGTCATTTT
>HG-U133A: 222204_s_at; 161; 317; 345; Antisense;
GCTAGAGTCATTTTGAAGCTCAAGC
>HG-U133A: 222204_s_at; 49; 693; 373; Antisense;
TTCACTTTGTTTCTTACATGTGTAC
>HG-U133A: 222204_s_at; 203; 363; 414; Antisense;
GAAAATGGCCATCTTTAAGCATATT
>HG-U133A: 222204_s_at; 186; 675; 442; Antisense;
TTTCTGCCACTTTATTTAAAGGCAA
>HG-U133A: 222204_s_at; 156; 701; 507; Antisense;
TTCCTCTTTTCCAGGGCTTTGTATG
>HG-U133A: 222204_s_at; 469; 279; 517; Antisense;
CCAGGGCTTTGTATGCACTTGTATA
>HG-U133A: 222204_s_at; 273; 449; 559; Antisense;
GTAGAGTTTGAATTTCAGTCTGTAA
>HG-U133A: 222204_s_at; 114; 513; 682; Antisense;
GGTTGTCTTTTTAACTGCTGGCAAA
>HG-U133A: 222204_s_at; 446; 657; 762; Antisense;
TAGTAAGTGGGGTCTTTGTGGGTTG
>HG-U133A: 222204_s_at; 8; 157; 878; Antisense;
AATGACATGGTTAATCTGGAACTTA
>HG-U133A: 200660_at; 368; 615; 118; Antisense;
TCGCTCAGCTCCAACATGGCAAAAA
>HG-U133A: 200660_at; 2; 167; 16; Antisense;
CAAGGCTGGGCCGGGAAGGGCGTGG
>HG-U133A: 200660_at; 587; 47; 212; Antisense;
AGGATGGTTATAACTACACTCTCTC
>HG-U133A: 200660_at; 133; 651; 226; Antisense;
TACACTCTCTCCAAGACAGAGTTCC
>HG-U133A: 200660_at; 34; 161; 262; Antisense;
AATACAGAACTAGCTGCCTTCACAA
>HG-U133A: 200660_at; 542; 237; 27; Antisense;
CGGGAAGGGCGTGGGTTGAGGAGAG
>HG-U133A: 200660_at; 431; 559; 303; Antisense;
TGGTGTCCTTGACCGCATGATGAAG
>HG-U133A: 200660_at; 300; 351; 324; Antisense;
GAAGAAACTGGACACCAACAGTGAT
>HG-U133A: 200660_at; 111; 477; 344; Antisense;
GTGATGGTCAGCTAGATTTCTCAGA
>HG-U133A: 200660_at; 370; 511; 40; Antisense;
GGTTGAGGAGAGGCTCCAGACCCGC
>HG-U133A: 200660_at; 25; 701; 432; Antisense;
TTCCCAGAAGCGGACCTGAGGACCC
>HG-U133A: 203535_at; 654; 183; 108; Antisense;
CACCTTCCACCAATACTCTGTGAAG
>HG-U133A: 203535_at; 113; 333; 273; Antisense;
GCAGCTGAGCTTCGAGGAGTTCATC
>HG-U133A: 203535_at; 305; 411; 303; Antisense;
GATGGCGAGGCTAACCTGGGCCTCC
>HG-U133A: 203535_at; 296; 417; 336; Antisense;
GATGCACGAGGGTGACGAGGGCCCT
>HG-U133A: 203535_at; 59; 259; 357; Antisense;
CCCTGGCCACCACCATAAGCCAGGC
>HG-U133A: 203535_at; 216; 387; 403; Antisense;
GACCACAGTGGCCAAGATCACAGTG
>HG-U133A: 203535_at; 316; 181; 430; Antisense;
CACGGCCATGGCCACAGTCATGGTG
>HG-U133A: 203535_at; 18; 97; 458; Antisense;
ACGGCCACAGGCCACTAATCAGGAG
>HG-U133A: 203535_at; 35; 501; 518; Antisense;
GGGGCCTGTTATGTCAAACTGTCTT
>HG-U133A: 203535_at; 618; 335; 65; Antisense;
GCAAAATGTCGCAGCTGGAACGCAA
>HG-U133A: 203535_at; 475; 531; 81; Antisense;
GGAACGCAACATAGAGACCATCATC
>HG-U133A: 204351_at; 422; 499; 123; Antisense;
GGGGGAGCTCAAGGTGCTGATGGAG
>HG-U133A: 204351_at; 259; 497; 16; Antisense;
GGGTCTGAATCTAGCACCATGACGG
>HG-U133A: 204351_at; 276; 419; 187; Antisense;
GATGCCGTGGATAAATTGCTCAAGG
>HG-U133A: 204351_at; 382; 385; 211; Antisense;
GACCTGGACGCCAATGGAGATGCCC
>HG-U133A: 204351_at; 68; 503; 237; Antisense;
GGTGGACTTCAGTGAGTTCATCGTG
>HG-U133A: 204351_at; 121; 479; 259; Antisense;
GTGTTCGTGGCTGCAATCACGTCTG
>HG-U133A: 204351_at; 590; 231; 278; Antisense;
CGTCTGCCTGTCACAAGTACTTTGA
>HG-U133A: 204351_at; 291; 379; 36; Antisense;
GACGGAACTAGAGACAGCCATGGGC
>HG-U133A: 204351_at; 282; 667; 379; Antisense;
TTTGTTGGCAATTATTCCCCTAGGC
>HG-U133A: 204351_at; 1; 273; 397; Antisense;
CCTAGGCTGAGCCTGCTCATGTACC
>HG-U133A: 204351_at; 283; 421; 63; Antisense;
GATCATAGACGTCTTTTCCCGATAT
>HG-U133A: 201825_s_at; 382; 43; 1296; Antisense;
AGGCAACTTCTCATAAAATTCCCAT
>HG-U133A: 201825_s_at; 165; 647; 1309; Antisense;
TAAAATTCCCATGGTTCTTCTCCTT
>HG-U133A: 201825_s_at; 90; 627; 1324; Antisense;
TCTTCTCCTTTGGCTATTTTTCAAA
>HG-U133A: 201825_s_at; 25; 273; 1381; Antisense;
CCTCATTCACGCTGACATTCTTTGG
>HG-U133A: 201825_s_at; 381; 515; 1410; Antisense;
GGATACAGCCAAGGCACTGGTACAG
>HG-U133A: 201825_s_at; 707; 385; 1477; Antisense;
GACCAGAGGCTGGCTATGTGGCTAC
>HG-U133A: 201825_s_at; 260; 547; 1495; Antisense;
TGGCTACCCCCATAGCTATGGTTCA
>HG-U133A: 201825_s_at; 328; 559; 1513; Antisense;
TGGTTCAGGCAGCCATGACTCTTCT
>HG-U133A: 201825_s_at; 194; 219; 1536; Antisense;
CTAAGTGATGCTTCTCATCTGCCTA
>HG-U133A: 201825_s_at; 66; 221; 1549; Antisense;
CTCATCTGCCTAAGGCGGGCGGGGT
>HG-U133A: 201825_s_at; 507; 369; 1734; Antisense;
GAAATTCTTCTGTAAGCCTGTCTGA
>HG-U133A: 218793_s_at; 490; 669; 2049; Antisense;
TATTTGCCATCATTAGTACCTCTCA
>HG-U133A: 218793_s_at; 333; 659; 2062; Antisense;
TAGTACCTCTCAACTTACTTTTTAG
>HG-U133A: 218793_s_at; 360; 163; 2166; Antisense;
AATTCTGAGCCATTAATCCTGCTAC
>HG-U133A: 218793_s_at; 120; 293; 2174; Antisense;
GCCATTAATCCTGCTACACTTTGAA
>HG-U133A: 218793_s_at; 544; 607; 2182; Antisense;
TCCTGCTACACTTTGAATGATACAT
>HG-U133A: 218793_s_at; 601; 189; 2212; Antisense;
CAGACTAATCTTTGGGGGCTTTATT
>HG-U133A: 218793_s_at; 569; 131; 2261; Antisense;
AACATGTTCAACACTATTATTTTGT
>HG-U133A: 218793_s_at; 370; 391; 2336; Antisense;
GAGCTATGAGAATTGGTGCTATCAC
>HG-U133A: 218793_s_at; 35; 507; 2350; Antisense;
GGTGCTATCACCATTAGCTATTTGC
>HG-U133A: 218793_s_at; 40; 689; 2363; Antisense;
TTAGCTATTTGCTGTAATGTCAAGA
>HG-U133A: 218793_s_at; 287; 87; 2396; Antisense;
ACCAGATGCAAGAATGTACCTTTTC
>HG-U133A: 204563_at; 278; 269; 1778; Antisense;
CCTCGCCGTCTGTGAATTGGACCAT
>HG-U133A: 204563_at; 612; 525; 1796; Antisense;
GGACCATCCTATTTAACTGGCTTCA
>HG-U133A: 204563_at; 411; 681; 1850; Antisense;
TTTTCAGTTGGCTGACTTCCACACC
>HG-U133A: 204563_at; 253; 285; 1868; Antisense;
CCACACCTAGCATCTCATGAGTGCC
>HG-U133A: 204563_at; 629; 657; 1917; Antisense;
TAGCCTGCGCTGTTTTTTAGTTTGG
>HG-U133A: 204563_at; 303; 677; 1959; Antisense;
TTTATGAGACCCATTCCTATTTCTT
>HG-U133A: 204563_at; 604; 457; 1987; Antisense;
GTCAATGTTTCTTTTATCACGATAT
>HG-U133A: 204563_at; 522; 383; 2140; Antisense;
GACCTTTTATCCACTTACCTAGATT
>HG-U133A: 204563_at; 5; 183; 2206; Antisense;
CACCACTTCTTTTATAACTAGTCCT
>HG-U133A: 204563_at; 393; 657; 2224; Antisense;
TAGTCCTTTACTAATCCAACCCATG
>HG-U133A: 204563_at; 105; 223; 2257; Antisense;
CTCTTCCTGGCTTCTTACTGAAAGG
>HG-U133A: 209879_at; 434; 167; 1763; Antisense;
CAAGGAAGATGGAGCTCCCCCATCC
>HG-U133A: 209879_at; 691; 179; 1794; Antisense;
CACTGCACTGCCATTGTCTTTTGGT
>HG-U133A: 209879_at; 456; 707; 1807; Antisense;
TTGTCTTTTGGTTGCCATGGTCACC
>HG-U133A: 209879_at; 650; 473; 1871; Antisense;
GTGACGGACTTCTGAGGCTGTTTCC
>HG-U133A: 209879_at; 699; 605; 1902; Antisense;
TCCTCTGACTTGGGGCAGCTTGGGT
>HG-U133A: 209879_at; 692; 497; 1956; Antisense;
GGGTGAGGTTCAGCCTGTGAGGGCT
>HG-U133A: 209879_at; 504; 547; 1996; Antisense;
GGCCCAAAGGGCAGACCTTTCTTTG
>HG-U133A: 209879_at; 103; 483; 2026; Antisense;
GTGTGGACCAAGGAGCTTCCATCTA
>HG-U133A: 209879_at; 532; 603; 2043; Antisense;
TCCATCTAGTGACAAGTGACCCCCA
>HG-U133A: 209879_at; 25; 605; 2101; Antisense;
TCCAGGGTGGACTCTGTCTTGTTCA
>HG-U133A: 209879_at; 386; 437; 2121; Antisense;
GTTCACTGCAGTATCCCAACTGCAG
>HG-U133A: 201585_s_at; 217; 375; 2437; Antisense;
GACATGCGTACTGAGCGCTTTGGGC
>HG-U133A: 201585_s_at; 606; 301; 2451; Antisense;
GCGCTTTGGGCAGGGAGGTGCGGGG
>HG-U133A: 201585_s_at; 670; 487; 2473; Antisense;
GGGCCTGTGGGTGGACAGGGTCCTA
>HG-U133A: 201585_s_at; 284; 493; 2490; Antisense;
GGGTCCTAGAGGAATGGGGCCTGGA
>HG-U133A: 201585_s_at; 132; 3; 2503; Antisense;
ATGGGGCCTGGAACTCCAGCAGGAT
>HG-U133A: 201585_s_at; 705; 391; 2540; Antisense;
GAGAAGAGTACGAAGGCCCAAACAA
>HG-U133A: 201585_s_at; 523; 137; 2567; Antisense;
AACCCCGATTTTAGATGTGATATTT
>HG-U133A: 201585_s_at; 655; 685; 2590; Antisense;
TTAGGCTTTCATTCCAGTTTGTTTT
>HG-U133A: 201585_s_at; 42; 41; 2677; Antisense;
ATGGATGTTAGCAGTTTATTGACCT
>HG-U133A: 201585_s_at; 429; 37; 2812; Antisense;
ATGTCCCTCAAGTTTATGGCAGTGT
>HG-U133A: 201585_s_at; 495; 479; 2833; Antisense;
GTGTACCTTGTGCCACTGAATTTCC
>HG-U133A: 214016_s_at; 315; 431; 740; Antisense;
GTTGGCTGATATTGGAGTGCTCATT
>HG-U133A: 214016_s_at; 583; 425; 747; Antisense;
GATATTGGAGTGCTCATTCACATGA
>HG-U133A: 214016_s_at; 103; 405; 754; Antisense;
GAGTGCTCATTCACATGAAGTGGAT
>HG-U133A: 214016_s_at; 378; 17; 777; Antisense;
ATAGATACTTCTCAAGACATCACAC
>HG-U133A: 214016_s_at; 620; 101; 783; Antisense;
ACTTCTCAAGACATCACACAGCGTG
>HG-U133A: 214016_s_at; 595; 375; 792; Antisense;
GACATCACACAGCGTGAGTCAATCA
>HG-U133A: 214016_s_at; 347; 305; 803; Antisense;
GCGTGAGTCAATCAAGGAGGGAAGC
>HG-U133A: 214016_s_at; 36; 521; 818; Antisense;
GGAGGGAAGCCACAAGCAGACTGAC
>HG-U133A: 214016_s_at; 164; 289; 826; Antisense;
GCCACAAGCAGACTGACAACGTTTC
>HG-U133A: 214016_s_at; 198; 157; 906; Antisense;
AATGAACGTTTCATTCTCGTTAATA
>HG-U133A: 214016_s_at; 455; 439; 913; Antisense;
GTTTCATTCTCGTTAATAAAGGCAT
>HG-U133A: 221768_at; 475; 389; 1413; Antisense;
GAGCTGATGTTAAAACTCATTTGGT
>HG-U133A: 221768_at; 674; 219; 1428; Antisense;
CTCATTTGGTGAGGTCAACGTTGTC
>HG-U133A: 221768_at; 413; 457; 1441; Antisense;
GTCAACGTTGTCACATACCTTCACA
>HG-U133A: 221768_at; 459; 495; 1469; Antisense;
GGGATAGTATATTTTGGGTTGCAGT
>HG-U133A: 221768_at; 173; 637; 1493; Antisense;
TCAAACTTGTGCTCAGACTGGTGAA
>HG-U133A: 221768_at; 368; 441; 1555; Antisense;
GTTTTCATTCTAATTCAGGTGTCTA
>HG-U133A: 221768_at; 544; 7; 1567; Antisense;
ATTCAGGTGTCTACTTATTTTATGT
>HG-U133A: 221768_at; 684; 257; 1612; Antisense;
CCCCCACCATGAAGTTTCTTCCTAT
>HG-U133A: 221768_at; 321; 679; 1640; Antisense;
TTTATGCTGTAACTTACCCCCAATC
>HG-U133A: 221768_at; 634; 687; 1653; Antisense;
TTACCCCCAATCTTTATCTCTGGAT
>HG-U133A: 221768_at; 571; 433; 1699; Antisense;
GTTGACTAGCATTTTCAAACCTTTA
>HG-U133A: 212721_at; 596; 401; 2963; Antisense;
GAGTTTAAGATACAGGTCATCCATC
>HG-U133A: 212721_at; 92; 461; 2978; Antisense;
GTCATCCATCATTCTTAGGCTCACT
>HG-U133A: 212721_at; 675; 625; 2986; Antisense;
TCATTCTTAGGCTCACTTTTTACAG
>HG-U133A: 212721_at; 81; 571; 3046; Antisense;
TGTTTTTCCCCAGTACTATAACTTG
>HG-U133A: 212721_at; 278; 21; 3063; Antisense;
ATAACTTGTGGTTTCTGAACTCATT
>HG-U133A: 212721_at; 456; 631; 3171; Antisense;
TCAGATTACTCAGTTGCCTTACCTC
>HG-U133A: 212721_at; 530; 633; 3180; Antisense;
TCAGTTGCCTTACCTCATGGGAAGA
>HG-U133A: 212721_at; 33; 77; 3234; Antisense;
AGCATGTTAGTTAC1TGGTTTCAAC
>HG-U133A: 212721_at; 2; 347; 3309; Antisense;
GAATGGAAAGAGTTGCCCTTGTTGC
>HG-U133A: 212721_at; 409; 295; 3344; Antisense;
GCCTGATTTGATTATGAAGCTGCTT
>HG-U133A: 212721_at; 389; 141; 3360; Antisense;
AAGCTGCTTAATCACTCTTCATGTG
>HG-U133A: 204790_at; 326; 51; 2541; Antisense;
AGGGACATGCTTAGCAGTCCCCTTC
>HG-U133A: 204790_at; 373; 347; 2573; Antisense;
GAAGGATTTGGTCCGTCATAACCCA
>HG-U133A: 204790_at; 483; 17; 2590; Antisense;
ATAACCCAAGGTACCATCCTAGGCT
>HG-U133A: 204790_at; 308; 27; 2605; Antisense;
ATCCTAGGCTGACACCTAACTCTTC
>HG-U133A: 204790_at; 29; 213; 2629; Antisense;
CTTTCATTTCTTCTACAACTCATAC
>HG-U133A: 204790_at; 88; 693; 2636; Antisense;
TTCTTCTACAACTCATACACTCGTA
>HG-U133A: 204790_at; 170; 651; 2651; Antisense;
TACACTCGTATGATACTTCGACACT
>HG-U133A: 204790_at; 137; 211; 2666; Antisense;
CTTCGACACTGTTCTTAGCTCAATG
>HG-U133A: 204790_at; 468; 79; 2682; Antisense;
AGCTCAATGAGCATGTTTAGACTTT
>HG-U133A: 204790_at; 247; 561; 3016; Antisense;
TGGTGTTTTTTCCTATGGGTGTTAT
>HG-U133A: 204790_at; 660; 37; 3030; Antisense;
ATGGGTGTTATCACCTAGCTGAATG
>HG-U133A: 208012_x_at; 595; 635; 247; Antisense;
TCACCTGGGCATGGCATCCAAGAGA
>HG-U133A: 208012_x_at; 195; 361; 303; Antisense;
GAAAGACGACTCAACCTGGAACTCA
>HG-U133A: 208012_x_at; 519; 217; 359; Antisense;
CTAAATGTGCCCGAAAGTCCAGATC
>HG-U133A: 208012_x_at; 422; 549; 479; Antisense;
TGGATTTTCACTGTTCTAAGTCCCC
>HG-U133A: 208012_x_at; 603; 259; 501; Antisense;
CCCCGTGACCTGTGGTGAGGCGAAA
>HG-U133A: 208012_x_at; 293; 95; 554; Antisense;
ACGGATCCTCAGTGAAGTGCATTCG
>HG-U133A: 208012_x_at; 61; 535; 673; Antisense;
GGAATGACCCTAGGAGAGCTGCTGA
>HG-U133A: 208012_x_at; 384; 213; 706; Antisense;
CTTTTGCTCTGTCCTCCAAGAATAA
>HG-U133A: 208012_x_at; 420; 339; 752; Antisense;
GCAAGTGAATTTCTACTACCCTCTC
>HG-U133A: 208012_x_at; 398; 271; 771; Antisense;
CCTCTCAGTCACCATGTTGCAGACT
>HG-U133A: 208012_x_at; 362; 591; 788; Antisense;
TGCAGACTTTCCCTGTCTGGAGGCT
>HG-U133A: 209761_s_at; 140; 691; 493; Antisense;
TTCAGTCAAATTAACCTGCGTGAAT
>HG-U133A: 209761_s_at; 145; 137; 505; Antisense;
AACCTGCGTGAATATCCCAATCTGG
>HG-U133A: 209761_s_at; 285; 25; 518; Antisense;
ATCCCAATCTGGTGACGATTTACAG
>HG-U133A: 209761_s_at; 42; 109; 539; Antisense;
ACAGAAGCTTCAAACGTGTTGGTGC
>HG-U133A: 209761_s_at; 130; 319; 545; Antisense;
GCTTCAAACGTGTTGGTGCTTCCTA
>HG-U133A: 209761_s_at; 501; 427; 556; Antisense;
GTTGGTGCTTCCTATGAACGGCAGA
>HG-U133A: 209761_s_at; 361; 273; 566; Antisense;
CCTATGAACGGCAGAGCAGAGACAC
>HG-U133A: 209761_s_at; 200; 191; 582; Antisense;
CAGAGACACACCAATCCTACTTGAA
>HG-U133A: 209761_s_at; 627; 69; 585; Antisense;
AGACACACCAATCCTACTTGAAGCC
>HG-U133A: 209761_s_at; 589; 83; 606; Antisense;
AGCCCCAACTGGCCTAGCAGAAGGA
>HG-U133A: 209761_s_at; 214; 183; 690; Antisense;
CACCCTGTGCGCCAAGAGTCAGTGA
>HG-U133A: 209762_x_at; 56; 515; 1460; Antisense;
GGATTTTCACTGTTCTAAGCTCCCC
>HG-U133A: 209762_x_at; 602; 259; 1481; Antisense;
CCCCGTGACCTGTGGTGAGGCGAAA
>HG-U133A: 209762_x_at; 292; 95; 1534; Antisense;
ACGGATCCTCAGTGAAGTGCATTCG
>HG-U133A: 209762_x_at; 585; 355; 1654; Antisense;
GAACGACCCTAGGAGAGCTGCTGAA
>HG-U133A: 209762_x_at; 694; 351; 1676; Antisense;
GAAGAGTGGACTTTGCTCTGTCCTC
>HG-U133A: 209762_x_at; 419; 339; 1731; Antisense;
GCAAGTGAATTTCTACTACCCTCTC
>HG-U133A: 209762_x_at; 397; 271; 1750; Antisense;
CCTCTCAGTCACCATGTTGCAGACT
>HG-U133A: 209762_x_at; 361; 591; 1767; Antisense;
TGCAGACTTTCCCTGTCTGGAGGCT
>HG-U133A: 209762_x_at; 450; 411; 1786; Antisense;
GAGGCTCACCTTAGAGCTTCTGAGT
>HG-U133A: 209762_x_at; 77; 581; 1806; Antisense;
TGAGTTTCCAAGCTCTGAGTCACCT
>HG-U133A: 209762_x_at; 488; 635; 1825; Antisense;
TCACCTCCACATTTGGGCATGGCAT
>HG-U133A: 201239_s_at; 581; 557; 275; Antisense;
TGGTCTAATTGATGGTCGCCTCACC
>HG-U133A: 201239_s_at; 305; 201; 299; Antisense;
CATCTGTACAATCTCCTGTTTCTTT
>HG-U133A: 201239_s_at; 688; 227; 311; Antisense;
CTCCTGTTTCTTTGCCATAGTGGCT
>HG-U133A: 201239_s_at; 9; 671; 340; Antisense;
TTTGGGA1TATATGCACCCCTTTCC
>HG-U133A: 201239_s_at; 547; 601; 362; Antisense;
TCCAGAGTCCAAACCCGTTTTGGCT
>HG-U133A: 201239_s_at; 642; 709; 381; Antisense;
TTGGCTTTGTGTGTCATATCCTATT
>HG-U133A: 201239_s_at; 498; 393; 447; Antisense;
GAGAAGAGCATCTTTCTCGTGGCCC
>HG-U133A: 201239_s_at; 7; 617; 461; Antisense;
TCTCGTGGCCCACAGGAAAGATCCT
>HG-U133A: 201239_s_at; 690; 415; 501; Antisense;
GATGATATTTGGCAGCTGTCCTCCA
>HG-U133A: 201239_s_at; 18; 357; 597; Antisense;
GAAGCCGAGTTCACAAAGTCCATTG
>HG-U133A: 201239_s_at; 328; 603; 678; Antisense;
TCCAGGCTCCATGACAGTCTTGCCA
>HG-U133A: 212060_at; 11; 143; 3785; Antisense;
AAGACTAGGTAGATATGGCATGGCG
>HG-U133A: 212060_at; 98; 277; 3867; Antisense;
CCATACATCCAACCCATGTTCTGAG
>HG-U133A: 212060_at; 300; 171; 3876; Antisense;
CAACCCATGTTCTGAGCAACTACTT
>HG-U133A: 212060_at; 298; 581; 3888; Antisense;
TGAGCAACTACTTACTTTTAGGGGG
>HG-U133A: 212060_at; 314; 115; 3918; Antisense;
AAATATCTTTTCATTTCCTCTTCTA
>HG-U133A: 212060_at; 171; 11; 3971; Antisense;
ATTTTCTAACAAGGTTTGGCCATAG
>HG-U133A: 212060_at; 639; 639; 4025; Antisense;
TAATCTTCTGTAGGCTATCTTTCAA
>HG-U133A: 212060_at; 114; 393; 4121; Antisense;
GAGACTTGGGTTTAGTTATAGCTTT
>HG-U133A: 212060_at; 680; 101; 4180; Antisense;
ACTTCGTATCTAATGGTTTGTAAAT
>HG-U133A: 212060_at; 601; 645; 4226; Antisense;
TAAACCATTTGCAGAGTTGAACTCT
>HG-U133A: 212060_at; 473; 155; 4300; Antisense;
AATGTTGGTCATAATACTGCTATAA
>HG-U133A: 202557_at 461; 187; 3413; Antisense;
CAGCTCATCTCATGTCCTGAAGTTG
>HG-U133A: 202557_at 343; 375; 3471; Antisense;
GACAGTGTTGGAATTTGGAGGCAGT
>HG-U133A: 202557_at; 281; 411; 3488; Antisense;
GAGGCAGTAGTTGAGCATATTCTCT
>HG-U133A: 202557_at; 568; 7; 3506; Antisense;
ATTCTCTAGTATATAGCTACACCTT
>HG-U133A: 202557_at; 676; 461; 3548; Antisense;
GTCTTCAATCATATTTTAGTGGGCT
>HG-U133A: 202557_at; 625; 57; 3691; Antisense;
AGTTGTACATTTAGCCAGTGTTATT
>HG-U133A: 202557_at; 248; 35; 3794; Antisense;
ATGTTTTGGTACTGTGTTTTCACTC
>HG-U133A: 202557_at; 335; 479; 3807; Antisense;
GTGTTTTCACTCAAACCACTGACTT
>HG-U133A: 202557_at; 159; 87; 3821; Antisense;
ACCACTGACTTAACAGATACTGCTG
>HG-U133A: 202557_at; 339; 423; 3836; Antisense;
GATACTGCTGTGTATAACATGTACT
>HG-U133A: 202557_at; 229; 431; 3887; Antisense;
GATTGTTCCTCTTATATTTGTGTGT
>HG-U133A: 208762_at; 478; 569; 1214; Antisense;
TGTCAAAAATCGTACTAATGCTTAT
>HG-U133A: 208762_at; 680; 149; 1278; Antisense;
AAGGTTTTCTTGCATAAATACTGGA
>HG-U133A: 208762_at; 161; 161; 1294; Antisense;
AATACTGGAAATTGCACATGGTACA
>HG-U133A: 208762_at; 629; 333; 1307; Antisense;
GCACATGGTACAAATTTTTTCTTCA
>HG-U133A: 208762_at; 527; 231; 1368; Antisense;
CTGAAAGTTACTGAAGTGCCTTCTG
>HG-U133A: 208762_at; 551; 349; 1380; Antisense;
GAAGTGCCTTCTGAATCAAGGATTT
>HG-U133A: 208762_at; 262; 511; 1399; Antisense;
GGATTTAATTAAGGCCACAATACCT
>HG-U133A: 208762_at; 21; 15; 1418; Antisense;
ATACCTTTTTAATACTCAGTGTTCT
>HG-U133A: 208762_at; 13; 127; 1453; Antisense;
AAAACTTGATATTCCCGTATGGTGC
>HG-U133A: 208762_at; 296; 425; 1460; Antisense;
GATATTCCCGTATGGTGCATATTTG
>HG-U133A: 208762_at; 168; 561; 1472; Antisense;
TGGTGCATATTTGATACAGGTACCC
>HG-U133A: 201463_s_at; 136; 41; 1066; Antisense;
ATGGAAAGTAGCGCATCCCTGAGGC
>HG-U133A: 201463_s_at; 382; 619; 1125; Antisense;
TCTGACTGCACGTGGCTTCTGATGA
>HG-U133A: 201463_s_at; 5; 173; 749; Antisense;
CAAAACCATTGTCATGGGCGCCTCC
>HG-U133A: 201463_s_at; 454; 603; 771; Antisense;
TCCTTCCGCAACACGGGCGAGATCA
>HG-U133A: 201463_s_at; 632; 21; 792; Antisense;
ATCAAAGCACTGGCCGGCTGTGACT
>HG-U133A: 201463_s_at; 677; 181; 829; Antisense;
CACCCAAGCTCCTGGGAGAGCTGCT
>HG-U133A: 201463_s_at; 314; 155; 920; Antisense;
AATCCACCTGGATGAGAAGTCTTTC
>HG-U133A: 201463_s_at; 123; 351; 935; Antisense;
GAAGTCTTTCCGTTGGTTGCACAAC
>HG-U133A: 201463_s_at; 303; 513; 949; Antisense;
GGTTGCACAACGAGGACCAGATGGC
>HG-U133A: 201463_s_at; 696; 311; 983; Antisense;
GCTCTCTGACGGGATCCGCAAGTTT
>HG-U133A: 201463_s_at; 576; 263; 998; Antisense;
CCGCAAGTTTGCCGCTGATGCAGTG
>HG-U133A: 202396_at; 269; 67; 3633; Antisense;
AGAGCATTTGTGGCTTGAACTTGCC
>HG-U133A: 202396_at; 304; 359; 3649; Antisense;
GAACTTGCCAGATGCAAATACCACA
>HG-U133A: 202396_at; 545; 341; 3757; Antisense;
GAATTCTTATCTTCCAGAGGCTACA
>HG-U133A: 202396_at; 420; 529; 3793; Antisense;
GGACAATACTTTTACCTTTGTCTCT
>HG-U133A: 202396_at; 112; 59; 3830; Antisense;
AGTTTTATTTGTTCACTTACGTGCT
>HG-U133A: 202396_at; 520; 635; 3842; Antisense;
TCACTTACGTGCTTTGATTATCCCC
>HG-U133A: 202396_at; 448; 427; 3857; Antisense;
GATTATCCCCTCTGAATTATAGACC
>HG-U133A: 202396_at; 67; 627; 3924; Antisense;
TCTTCTCAGGTATGGAACCACGGTC
>HG-U133A: 202396_at; 444; 355; 3938; Antisense;
GAACCACGGTCATAACTAACATGTT
>HG-U133A: 202396_at; 710; 367; 4034; Antisense;
GACAACAAATTACCTTTCTGGGTGT
>HG-U133A: 202396_at; 409; 623; 4060; Antisense;
TCTTGTAAACTATACTCCTGTTTGA
>HG-U133A: 201821_s_at; 638; 553; 375; Antisense;
TGGCATTCTCCTAGCTTTAATTGAA
>HG-U133A: 201821_s_at; 219; 347; 397; Antisense;
GAAGGAGCTGGTATCTTGTTGACAA
>HG-U133A: 201821_s_at; 368; 611; 472; Antisense;
TCCCAGTTGCCTTCAACTCAGTTAC
>HG-U133A: 201821_s_at; 574; 181; 503; Antisense;
CACCTTTTGGAGACTATCGACAATA
>HG-U133A: 201821_s_at; 17; 371; 521; Antisense;
GACAATATCAGTAGGACTTCTTTCC
>HG-U133A: 201821_s_at; 380; 625; 539; Antisense;
TCTTTCCTAGGATTTCTTTAACAGA
>HG-U133A: 201821_s_at; 444; 397; 566; Antisense;
GAGTTGTGGTTCGAGAAGGATTTCA
>HG-U133A: 201821_s_at; 692; 75; 633; Antisense;
AGCTATGGCCAATAGGCTATAAAGA
>HG-U133A: 201821_s_at; 17; 121; 653; Antisense;
AAAGAGACATTTAGCACTTTTTTCT
>HG-U133A: 201821_s_at; 582; 597; 864; Antisense;
TGCCTGGTTTTGTGTGTTCTGTTAT
>HG-U133A: 201821_s_at; 50; 307; 903; Antisense;
GCTGGTGGAACTTACTCTTTCTTTT
>HG-U133A: 220832_at; 226; 647; 2817; Antisense;
TAAAAGGCTACAGGTCTCTTTCCAC
>HG-U133A: 220832_at; 671; 671; 2835; Antisense;
TTTCCACATCCCAAACTTTCTATGA
>HG-U133A: 220832_at; 172; 373; 2876; Antisense;
GACACCAAAGATGCCTCTGTTACTG
>HG-U133A: 220832_at; 173; 311; 2917; Antisense;
GCTGCGCTACCACCTTGAAGAGAGC
>HG-U133A: 220832_at; 316; 169; 2947; Antisense;
CAAAAACGTTCTCCTTTGTCTAGAG
>HG-U133A: 220832_at; 10; 27; 3002; Antisense;
ATCGACAACCTCATGCAGAGCATCA
>HG-U133A: 220832_at; 590; 317; 3092; Antisense;
GCTTTTTACTTGGCTTTGCAGAGGC
>HG-U133A: 220832_at; 562; 525; 3157; Antisense;
GGAGCCAGTGTTACAGCATTCTCAG
>HG-U133A: 220832_at; 293; 705; 3185; Antisense;
TTGAGGCTACGGCAGCGGATCTGTA
>HG-U133A: 220832_at; 88; 307; 3199; Antisense;
GCGGATCTGTAAGAGCTCCATCCTC
>HG-U133A: 220832_at; 570; 601; 3222; Antisense;
TCCAGTGGCCTGACAACCCGAAGGC
>HG-U133A: 202643_s_at; 358; 623; 3316; Antisense;
TCTTTGGGTTATTACTGTCTTTACT
>HG-U133A: 202643_s_at; 203; 369; 3433; Antisense;
GAAATGCTGCCCTAGAAGTACAATA
>HG-U133A: 202643_s_at; 473; 695; 3484; Antisense;
TTCTGGTTGTTGTTGGGGCATGAGC
>HG-U133A: 202643_s_at; 346; 321; 3522; Antisense;
GCTTGCATAAACTCAACCAGCTGCC
>HG-U133A: 202643_s_at; 609; 51; 3554; Antisense;
AGGGAGCTCTAGTCCTTTTTGTGTA
>HG-U133A: 202643_s_at; 559; 397; 3668; Antisense;
GAGAGAACATCCTTGCTTTGAGTCA
>HG-U133A: 202643_s_at; 522; 489; 3699; Antisense;
GGGCAAGTTCCTGACCACAGGGAGT
>HG-U133A: 202643_s_at; 648; 493; 3718; Antisense;
GGGAGTAAATTGGCCTCTTTGATAC
>HG-U133A: 202643_s_at; 403; 625; 3733; Antisense;
TCTTTGATACACTTTTGCTTGCCTC
>HG-U133A: 202643_s_at; 649; 7; 3797; Antisense;
ATTCATCGATGTTTCGTGCTTCTCC
>HG-U133A: 202643_s_at; 656; 471; 3812; Antisense;
GTGCTTCTCCTTATGAAACTCCAGC
>HG-U133A: 202687_s_at; 58; 451; 466; Antisense;
GTAGCAGCTCACATAACTGGGACCA
>HG-U133A: 202687_s_at; 146; 207; 506; Antisense;
CATTGTCTTCTCCAAACTCCAAGAA
>HG-U133A: 202687_s_at; 591; 539; 542; Antisense;
TGGGCCGCAAAATAAACTCCTGGGA
>HG-U133A: 202687_s_at; 646; 489; 580; Antisense;
GGGCATTCATTCCTGAGCAACTTGC
>HG-U133A: 202687_s_at; 446; 681; 643; Antisense;
TTTTACTACATCTATTCCCAAACAT
>HG-U133A: 202687_s_at; 647; 613; 658; Antisense;
TCCCAAACATACTTTCGATTTCAGG
>HG-U133A: 202687_s_at; 229; 161; 737; Antisense;
AATACACAAGTTATCCTGACCCTAT
>HG-U133A: 202687_s_at; 666; 527; 814; Antisense;
GGACTCTATTCCATCTATCAAGGGG
>HG-U133A: 202687_s_at; 490; 581; 888; Antisense;
TGAGCACTTGATAGACATGGACCAT
>HG-U133A: 202687_s_at; 620; 523; 906; Antisense;
GGACCATGAAGCCAGTTTTTTCGGG
>HG-U133A: 202687_s_at; 608; 291; 931; Antisense;
GCCTTTTTAGTTGGCTAACTGACCT
>HG-U133A: 202688_at; 508; 223; 1181; Antisense;
CTCTACCTCATATCAGTTTGCTAGC
>HG-U133A: 202688_at; 422; 143; 1216; Antisense;
AAGACTGTCAGCTTCCAAACATTAA
>HG-U133A: 202688_at; 427; 33; 1240; Antisense;
ATGCAATGGTTAACATCTTCTGTCT
>HG-U133A: 202688_at; 241; 619; 1258; Antisense;
TCTGTCTTTATAATCTACTCCTTGT
>HG-U133A: 202688_at; 443; 639; 1268; Antisense;
TAATCTACTCCTTGTAAAGACTGTA
>HG-U133A: 202688_at; 297; 71; 1295; Antisense;
AGAAAGCGCAACAATCCATCTCTCA
>HG-U133A: 202688_at; 345; 29; 1308; Antisense;
ATCCATCTCTCAAGTAGTGTATCAC
>HG-U133A: 202688_at; 533; 53; 1323; Antisense;
AGTGTATCACAGTAGTAGCCTCCAG
>HG-U133A: 202688_at; 465; 635; 1329; Antisense;
TCACAGTAGTAGCCTCCAGGTTTCC
>HG-U133A: 202688_at; 504; 409; 1388; Antisense;
GAGGCACCACTAAAAGATCGCAGTT
>HG-U133A: 202688_at; 151; 29; 1404; Antisense;
ATCGCAGTTTGCCTGGTGCAGTGGC
>HG-U133A: 209500_x_at; 433; 199; 1114; Antisense;
CATGGAGCTCCGAATTCTTGCGTGT
>HG-U133A: 209500_x_at; 612; 321; 1170; Antisense;
GCATTGTTCAGACCTGGTCGGGGCC
>HG-U133A: 209500_x_at; 543; 613; 1187; Antisense;
TCGGGGCCCACTGGAAGCATCCAGA
>HG-U133A: 209500_x_at; 423; 639; 1286; Antisense;
TAGGGAAAACCCCTGGTTCTCCATG
>HG-U133A: 209500_x_at; 334; 285; 1347; Antisense;
CCACAAGAAGCCTTATCCTACGTCC
>HG-U133A: 209500_x_at; 324; 399; 1414; Antisense;
GAGATGTAGCTATTATGCGCCCGTC
>HG-U133A: 209500_x_at; 695; 211; 1438; Antisense;
CTACAGGGGGTGCCCGACGATGACG
>HG-U133A: 209500_x_at; 65; 469; 1463; Antisense;
GTGCCTTCGCAGTCAAATTACTCTT
>HG-U133A: 209500_x_at; 305; 701; 1541; Antisense;
TTCCAAGCCCTTCCGGGCTGGAACT
>HG-U133A: 209500_x_at; 699; 615; 1570; Antisense;
TCGGAGGAGCCTCGGGTGTATCGTA
>HG-U133A: 209500_x_at; 450; 235; 1624; Antisense;
CTGAGCTCTTCTTTCTGATCAAGCC
>HG-U133A: 212635_at; 343; 75; 2525; Antisense;
AGCAAATTGAGCTTGGGTGATTTTT
>HG-U133A: 212635_at; 345; 123; 2625; Antisense;
AAACGTGGTAAATCACTTCATATTA
>HG-U133A: 212635_at; 28; 57; 2696; Antisense;
AGTAGCATTAGCTTTAGTTACAAAT
>HG-U133A: 212635_at; 128; 515; 2727; Antisense;
GGATCTTTCTGCTGACAACTTAGGT
>HG-U133A: 212635_at; 421; 649; 2776; Antisense;
TAAATCTGATGTTTCCTGTACCTGC
>HG-U133A: 212635_at; 79; 235; 2791; Antisense;
CTGTACCTGCCACACTATGTTAGAA
>HG-U133A: 212635_at; 664; 37; 2815; Antisense;
ATGTGTCCTTCAAACATATCCTCCT
>HG-U133A: 212635_at; 681; 225; 2835; Antisense;
CTCCTGCAACTTCTCAAACTGTACT
>HG-U133A: 212635_at; 9; 629; 2871; Antisense;
TCTTGAAGTCTAACTCTGTGCTAAC
>HG-U133A: 212635_at; 628; 233; 2886; Antisense;
CTGTGCTAACAGATCTCCATTTTAA
>HG-U133A: 212635_at; 232; 63; 3051; Antisense;
AGATGTGAATGTTAATCACTGCTTG
>HG-U133A: 213158_at; 250; 669; 1655; Antisense;
TTTGGAAAAACCTTGCATACGCCTT
>HG-U133A: 213158_at; 426; 323; 1669; Antisense;
GCATACGCCTTTTCTATCAAGTGCT
>HG-U133A: 213158_at; 294; 105; 1743; Antisense;
ACAGTATCCTTACCTGCCATTTAAT
>HG-U133A: 213158_at; 510; 89; 1754; Antisense;
ACCTGCCATTTAATATTAGCCTCGT
>HG-U133A: 213158_at; 609; 265; 1773; Antisense;
CCTCGTATTTTTCTCACGTATATTT
>HG-U133A: 213158_at; 248; 95; 1788; Antisense;
ACGTATATTTACCTGTGACTTGTAT
>HG-U133A: 213158_at; 351; 135; 1857; Antisense;
AACTGTAGCGCTTCATTATACTATT
>HG-U133A: 213158_at; 305; 59; 1921; Antisense;
AGTTTTATCTCTTGCATATACTTTA
>HG-U133A: 213158_at; 509; 647; 2070; Antisense;
TAAATGTTACCAGCACTTTTTTTGT
>HG-U133A: 213158_at; 184; 569; 2092; Antisense;
TGTAAGTTTCACTTTCCGAGGTATT
>HG-U133A: 213158_at; 697; 509; 2111; Antisense;
GGTATTGTACAAGTTCACACTGTTT
>HG-U133A: 203721_s_at; 620; 7; 1331; Antisense;
ATTAAGCATTGCCACATCTAGGAAT
>HG-U133A: 203721_s_at; 99; 513; 1463; Antisense;
GGTTACAGGTGTTACTTCTCTGACC
>HG-U133A: 203721_s_at; 73; 623; 1479; Antisense;
TCTCTGACCTTCAATCCTACTACAG
>HG-U133A: 203721_s_at; 103; 355; 1536; Antisense;
GAAGCAGTCAGATTGGTTCATCTTC
>HG-U133A: 203721_s_at; 172; 451; 1567; Antisense;
GTACAGTATTTTCAAACTTCCCAGT
>HG-U133A: 203721_s_at; 427; 435; 1617; Antisense;
GTTCATACCATGGATTTTTCTCCGA
>HG-U133A: 203721_s_at; 439; 349; 1642; Antisense;
GAAGTGGATACTTTGCCTTGGGGAA
>HG-U133A: 203721_s_at; 564; 153; 1676; Antisense;
AAGGCCCTGATGTATAGGTTGCACC
>HG-U133A: 203721_s_at; 272; 663; 1690; Antisense;
TAGGTTGCACCATTACTCAGACTTC
>HG-U133A: 203721_s_at; 702; 393; 1749; Antisense;
GAGAAGCCTGTCTTGATATATCATC
>HG-U133A: 203721_s_at; 498; 419; 1821; Antisense;
GATCCAGCTGTGCTTAAGAGCCAGT
>HG-U133A: 205922_at; 349; 515; 1428; Antisense;
GGATCATCTGGGCCTATACTAACAG
>HG-U133A: 205922_at; 294; 483; 1501; Antisense;
GTGGGACCAGCAATTCAGCAATAAC
>HG-U133A: 205922_at; 283; 439; 1580; Antisense;
GTTATAGGGGCGTCTCTTTATCACT
>HG-U133A: 205922_at; 391; 213; 1595; Antisense;
CTTTATCACTCAGCTTCTGCATCAT
>HG-U133A: 205922_at; 685; 209; 1608; Antisense;
CTTCTGCATCATACGCTTGGCTGAA
>HG-U133A: 205922_at; 14; 545; 1626; Antisense;
GGCTGAATGTGTTTATCGGCTTCCC
>HG-U133A: 205922_at; 709; 155; 1820; Antisense;
AATGAAGATCAAACTCCAGCTCCAG
>HG-U133A: 205922_at; 505; 281; 1841; Antisense;
CCAGCCTCATTTTGCTTGAGACTTT
>HG-U133A: 205922_at; 147; 493; 1892; Antisense;
GGGAGTGAGGAGTTTCAGGGCCATT
>HG-U133A: 205922_at; 458; 293; 1911; Antisense;
GCCATTGAAACATAGCTGTGCCCTT
>HG-U133A: 205922_at; 617; 511; 1965; Antisense;
GGTTTATGACTGAATTCCCTTTGAC
>HG-U133A: 220528_at; 667; 359; 1190; Antisense;
GAACAGACGAGATCTATGCCCTAGG
>HG-U133A: 220528_at; 230; 31; 1205; Antisense;
ATGCCCTAGGTGCTTTTGATGGACT
>HG-U133A: 220528_at; 134; 357; 1309; Antisense;
GAACCTGTGGGGTCAGCTTTTACCA
>HG-U133A: 220528_at; 290; 169; 1332; Antisense;
CAAGTTTGAAGACTTCTCCCTCAGT
>HG-U133A: 220528_at; 709; 469; 1364; Antisense;
TTGGAACGCGTTATGTTTTCCCACA
>HG-U133A: 220528_at; 527; 619; 1395; Antisense;
TCTAAGTGGGAGTCAGCTTGCCCCT
>HG-U133A: 220528_at; 288; 707; 1453; Antisense;
TTGAGGAGCCGAAGTGGAGCCCCTT
>HG-U133A: 220528_at; 695; 703; 1477; Antisense;
TTGCCTGTCTTAGTTATGGCCCTGT
>HG-U133A: 220528_at; 312; 269; 1525; Antisense;
CCTCCACGCTTAGGGCAGGGATCTG
>HG-U133A: 220528_at; 15; 369; 1551; Antisense;
GAAATTCCAGTGATCTCCTTTAGCA
>HG-U133A: 220528_at; 594; 329; 1573; Antisense;
GCAGAGCCCTTTTAGGATTAGCCTG
>HG-U133A: 204847_at; 229; 227; 4494; Antisense;
CTCCTCAAGCTATCCAATTTTCTGA
>HG-U133A: 204847_at; 408; 643; 4528; Antisense;
TAACCATGAGAGATGCCACATTTCT
>HG-U133A: 204847_at; 97; 693; 4549; Antisense;
TTCTCTCTGGGAAACTACCACTCAA
>HG-U133A: 204847_at; 600; 329; 4642; Antisense;
GCAGATCACATGTAAATCATTCCTA
>HG-U133A: 204847_at; 196; 569; 4683; Antisense;
TGTGCCTTGATGTACATATATTACT
>HG-U133A: 204847_at; 533; 667; 4699; Antisense;
TATATTACTAAGTTGCCTCTCCCAG
>HG-U133A: 204847_at; 250; 39; 4759; Antisense;
ATGTGATAGCTGTGCATGCATTATA
>HG-U133A: 204847_at; 115; 81; 4817; Antisense;
AGCTGTGTGGCTGACTTTCAATTTT
>HG-U133A: 204847_at; 484; 705; 4852; Antisense;
TTGACATACAGCCCATAACTTTATA
>HG-U133A: 204847_at; 547; 101; 4869; Antisense;
ACTTTATAATGGCTGCTCATTTATC
>HG-U133A: 204847_at; 568; 201; 4961; Antisense;
CATCCTCTGTTGTTACTAGATTTAG
>HG-U133A: 203739_at; 503; 59; 5073; Antisense;
AGTTTTGCACTTTTATAGCCTATTT
>HG-U133A: 203739_at; 319; 111; 5108; Antisense;
ACACATTTGCAAGATGATTGACTCA
>HG-U133A: 203739_at; 519; 3; 5124; Antisense;
ATTGACTCAATCTTTGCCTAATCCA
>HG-U133A: 203739_at; 214; 703; 5137; Antisense;
TTGCCTAATCCAATGAGTGTTACAG
>HG-U133A: 203739_at; 471; 397; 5161; Antisense;
GAGAGCTTGCTGTGACTAGAACCAT
>HG-U133A: 203739_at; 327; 691; 5306; Antisense;
TTCAGATTTCTCTTTTTAACCACAT
>HG-U133A: 203739_at; 409; 699; 5359; Antisense;
TTCCTACAGCCCTTTGTACTTCAAA
>HG-U133A: 203739_at; 47; 17; 5384; Antisense;
ATATGTTTTTGTGTCCATCAGTATT
>HG-U133A: 203739_at; 165; 637; 5407; Antisense;
TTAACTATTGGTATACTACTGGTTT
>HG-U133A: 203739_at; 688; 53; 5469; Antisense;
AGAGGTACAATTCGTTGGATTTTTG
>HG-U133A: 203739_at; 1; 377; 5560; Antisense;
GACATTACGTGTTTTATTTATGATA
>HG-U133A: 209431_s_at; 374; 87; 3074; Antisense;
ACCATGGGGTGAGTGTCCTCCAAGA
>HG-U133A: 209431_s_at; 494; 319; 3151; Antisense;
GCTTGGAGGCGAGCATTTTCACTGC
>HG-U133A: 209431_s_at; 273; 693; 3168; Antisense;
TTCACTGCTAGGACAAGCTCAGCTG
>HG-U133A: 209431_s_at; 379; 427; 3230; Antisense;
GATTTTAACCATTCAACATGCTGTT
>HG-U133A: 209431_s_at; 257; 341; 3314; Antisense;
GAATTGCTACTGAAAGCTATCCCAG
>HG-U133A: 209431_s_at; 75; 319; 3329; Antisense;
GCTATCCCAGGTGATACAGAGCTCT
>HG-U133A: 209431_s_at; 236; 389; 3347; Antisense;
GAGCTCTTTGTAAACCGCAGTCACA
>HG-U133A: 209431_s_at; 602; 157; 3475; Antisense;
AATGCCAGTCTGGTCAGGGAAGTAG
>HG-U133A: 209431_s_at; 369; 279; 3522; Antisense;
CCAGGAAGGTGGGACAGCCGGCAGG
>HG-U133A: 209431_s_at; 164; 421; 3546; Antisense;
GTAGGGACATTGTGTACCTCAGTTG
>HG-U133A: 209431_s_at; 443; 479; 3557; Antisense;
GTGTACCTCAGTTGTGTCACATGTG
>HG-U133A: 213097_s_at; 24; 123; 1496; Antisense;
AAAGCTGTGAATCTGTTCCCTGCTG
>HG-U133A: 213097_s_at; 16; 701; 1511; Antisense;
TTCCCTGCTGGAACAAATTCAAGAT
>HG-U133A: 213097_s_at; 280; 403; 1697; Antisense;
GAGTGGTACCTCAAGCAGACAACGC
>HG-U133A: 213097_s_at; 518; 111; 1715; Antisense;
ACAACGCAACGCCTTCAGAACGATT
>HG-U133A: 213097_s_at; 580; 43; 1743; Antisense;
AGGTCCATATACAGACTTCACCCCT
>HG-U133A: 213097_s_at; 370; 377; 1756; Antisense;
GACTTCACCCCTTGGACAACAGAAG
>HG-U133A: 213097_s_at; 359; 111; 1797; Antisense;
ACAAGCTTTGAAAACATACCCAGTA
>HG-U133A: 213097_s_at; 514; 127; 1809; Antisense;
AACATACCCAGTAAATACACCTGAA
>HG-U133A: 213097_s_at; 503; 17; 1846; Antisense;
ATAGCAGAAGCGGTGCCTGGCAGGA
>HG-U133A: 213097_s_at 325; 347; 1875; Antisense;
GAAGGACTGCATGAAACGATACAAG
>HG-U133A: 213097_s_at; 607; 311; 1930; Antisense;
GCTGCTCAAGAACAAGTGCTGAATG
>HG-U133A: 221658_s_at; 159; 157; 2022; Antisense;
AATGCCCATGGTACTCCATGCATTC
>HG-U133A: 221658_s_at; 57; 593; 2057; Antisense;
TGCATGTCTGGACTCACGGAGCTCA
>HG-U133A: 221658_s_at; 542; 477; 2159; Antisense;
GTGTTGCAAGTTGGTCCACAGCATC
>HG-U133A: 221658_s_at; 324; 285; 2174; Antisense;
CCACAGCATCTCCGGGGCTTTGTGG
>HG-U133A: 221658_s_at; 383; 317; 2190; Antisense;
GCTTTGTGGGATCAGGGCATTGCCT
>HG-U133A: 221658_s_at 233; 349; 2265; Antisense;
GAAGTCCATATTGTTCCTTATCACC
>HG-U133A: 221658_s_at; 704; 547; 2357; Antisense;
GGCCCCTGGACGAAGGTCTGAATCC
>HG-U133A: 221658_s_at 354; 151; 2369; Antisense;
AAGGTCTGAATCCCGACTCTGATAC
>HG-U133A: 221658_s_at; 160; 317; 2437; Antisense;
GCTAGAGTTTCCTTATCCAGACAGT
>HG-U133A: 221658_s_at; 589; 367; 2486; Antisense;
GAAATTGGCGATGTCACCCGTGTAC
>HG-U133A: 221658_s_at; 267; 329; 2526; Antisense;
GCAGACCCTCAATAAACGTCAGCTT

TABLE 7
PROBESETS RESPONSIVE TO IL-13 STIMULATION

| A | B | C | D | E | F |
|---|---|---|---|---|---|
| Name | Gene Symbol | IM_IL13_2h_STQValue | IM_IL13_6h_STQValue | IM_IL13_12h_STQValue | IM_IL13_24h_STQValue |
| 1179_at | — | 0.300 | 0.540 | 0.007 | 0.263 |
| 32218_at | — | 0.007 | 0.044 | 0.137 | 0.075 |
| 32247_at | — | 0.493 | 0.233 | 0.521 | 0.039 |
| 1150_at | — | 0.055 | 0.002 | 0.001 | 0.001 |
| 1284_at | — | 0.021 | 0.011 | 0.028 | 0.031 |
| 40888_f_at | — | 0.271 | 0.545 | 0.285 | 0.025 |
| 953_g_at | — | 0.198 | 0.014 | 0.002 | 0.005 |
| 34145_at | — | 0.040 | 0.014 | 0.019 | 0.074 |
| 1173_g_at | — | 0.302 | 0.032 | 0.079 | 0.020 |
| 956_at | — | 0.117 | 0.015 | 0.102 | 0.015 |
| 1148_s_at | — | 0.326 | 0.477 | 0.049 | 0.043 |
| 38033_at | 38970 | 0.114 | 0.197 | 0.033 | 0.026 |
| 33173_g_at | 38971 | 0.359 | 0.030 | 0.060 | 0.008 |
| 160044_g_at | ACO2 | 0.427 | 0.125 | 0.069 | 0.022 |
| 40082_at | ACSL1 | 0.517 | 0.630 | 0.410 | 0.028 |
| 33881_at | ACSL3 | 0.513 | 0.307 | 0.014 | 0.112 |
| 39330_s_at | ACTN1 | 0.204 | 0.052 | 0.036 | 0.016 |
| 41654_at | ADA | 0.418 | 0.300 | 0.273 | 0.020 |
| 907_at | ADA | 0.426 | 0.360 | 0.338 | 0.024 |
| 35479_at | ADAM28 | 0.221 | 0.013 | 0.014 | 0.005 |
| 34378_at | ADFP | 0.138 | 0.309 | 0.213 | 0.023 |
| 34777_at | ADM | 0.403 | 0.467 | 0.106 | 0.023 |
| 40821_at | AHCY | 0.448 | 0.144 | 0.183 | 0.050 |
| 40516_at | AHR | 0.423 | 0.002 | 0.025 | 0.101 |
| 40789_at | AK2 | 0.217 | 0.327 | 0.062 | 0.016 |
| 38780_at | AKR1A1 | 0.536 | 0.279 | 0.015 | 0.015 |
| 36589_at | AKR1B1 | 0.445 | 0.248 | 0.089 | 0.022 |
| 37015_at | ALDH1A1 | 0.266 | 0.245 | 0.038 | 0.000 |
| 38315_at | ALDH1A2 | 0.102 | 0.031 | 0.109 | 0.180 |
| 40685_at | ALDH3B1 | 0.042 | 0.138 | 0.230 | 0.403 |
| 37330_at | ALDH4A1 | 0.501 | 0.118 | 0.221 | 0.020 |
| 34636_at | ALOX15 | 0.229 | 0.000 | 0.005 | 0.001 |
| 307_at | ALOX5 | 0.192 | 0.036 | 0.123 | 0.053 |
| 37099_at | ALOX5AP | 0.510 | 0.269 | 0.123 | 0.012 |
| 678_at | ALPPL2 | 0.543 | 0.199 | 0.420 | 0.031 |
| 38417_at | AMPD2 | 0.081 | 0.017 | 0.084 | 0.007 |
| 39315_at | ANGPT1 | 0.559 | 0.072 | 0.021 | 0.099 |
| 36637_at | ANXA11 | 0.564 | 0.009 | 0.021 | 0.003 |
| 37647_at | AOAH | 0.153 | 0.236 | 0.007 | 0.001 |
| 41549_s_at | AP1S2 | 0.164 | 0.167 | 0.197 | 0.039 |
| 37669_s_at | ATP1B1 | 0.403 | 0.004 | 0.040 | 0.016 |
| 37992_s_at | ATP5D | 0.143 | 0.593 | 0.490 | 0.019 |
| 34811_at | ATP5G3 | 0.326 | 0.250 | 0.000 | 0.014 |
| 38751_i_at | ATP5I | 0.405 | 0.495 | 0.484 | 0.014 |
| 36142_at | ATXN1 | 0.378 | 0.008 | 0.084 | 0.001 |
| 39942_at | BATF | 0.017 | 0.130 | 0.519 | 0.365 |
| 37971_at | BAZ1A | 0.368 | 0.126 | 0.068 | 0.016 |
| 36812_at | BCAR3 | 0.163 | 0.003 | 0.008 | 0.036 |
| 32828_at | BCKDK | 0.217 | 0.004 | 0.095 | 0.009 |
| 41356_at | BCL11A | 0.227 | 0.167 | 0.227 | 0.030 |
| 2002_s_at | BCL2A1 | 0.375 | 0.592 | 0.219 | 0.005 |
| 40091_at | BCL6 | 0.274 | 0.002 | 0.008 | 0.012 |
| 32842_at | BCL7A | 0.139 | 0.015 | 0.047 | 0.024 |
| 40879_at | BICD2 | 0.043 | 0.402 | 0.537 | 0.424 |
| 32726_g_at | BID | 0.201 | 0.118 | 0.099 | 0.022 |
| 32618_at | BLVRA | 0.146 | 0.002 | 0.116 | 0.042 |
| 41732_at | BOLA2 | 0.351 | 0.070 | 0.227 | 0.030 |
| 35615_at | BOP1 /// | 0.248 | 0.033 | 0.352 | 0.305 |
| | LOC653119 | | | | |
| 33759_at | BPGM | 0.540 | 0.534 | 0.026 | 0.360 |
| 41639_at | BRRN1 | 0.160 | 0.111 | 0.012 | 0.024 |
| 32675_at | BST1 | 0.335 | 0.169 | 0.091 | 0.024 |
| 38760_f_at | BTN3A2 | 0.207 | 0.553 | 0.460 | 0.048 |
| 41415_at | BYSL | 0.125 | 0.200 | 0.019 | 0.056 |
| 39172_at | C10orf22 | 0.393 | 0.008 | 0.004 | 0.027 |
| 38652_at | C10orf26 | 0.137 | 0.055 | 0.001 | 0.016 |
| 38411_at | C11orf32 | 0.221 | 0.015 | 0.013 | 0.008 |
| 41437_at | C14orf109 | 0.159 | 0.348 | 0.106 | 0.027 |
| 38969_at | C19orf10 | 0.062 | 0.015 | 0.378 | 0.408 |
| 41409_at | C1orf38 | 0.032 | 0.484 | 0.538 | 0.372 |
| 37668_at | C1QBP | 0.146 | 0.284 | 0.000 | 0.011 |
| 33374_at | C2 | 0.325 | 0.335 | 0.582 | 0.047 |
| 32107_at | C21orf25 | 0.358 | 0.052 | 0.095 | 0.023 |
| 31927_s_at | C21orf33 | 0.445 | 0.108 | 0.302 | 0.020 |
| 32068_at | C3AR1 | 0.426 | 0.016 | 0.018 | 0.011 |
| 40175_at | C3orf40 | 0.160 | 0.319 | 0.398 | 0.038 |
| 39710_at | C5orf13 | 0.560 | 0.011 | 0.169 | 0.337 |
| 41696_at | C7orf24 | 0.518 | 0.055 | 0.170 | 0.014 |
| 34995_at | CALCRL | 0.436 | 0.072 | 0.027 | 0.008 |
| 38716_at | CAMKK2 | 0.018 | 0.660 | 0.194 | 0.421 |
| 574_s_at | CASP1 | 0.077 | 0.068 | 0.047 | 0.000 |
| 39320_at | CASP1 | 0.033 | 0.198 | 0.021 | 0.000 |
| 37162_at | CCDC6 | 0.330 | 0.324 | 0.253 | 0.036 |
| 34183_at | CCDC69 | 0.184 | 0.022 | 0.360 | 0.495 |
| 37454_at | CCL13 | 0.137 | 0.045 | 0.055 | 0.009 |
| 1183_at | CCL17 | 0.080 | 0.002 | 0.028 | 0.008 |
| 32128_at | CCL18 | 0.139 | 0.007 | 0.035 | 0.000 |
| 875_g_at | CCL2 | 0.539 | 0.295 | 0.070 | 0.028 |
| 34375_at | CCL2 | 0.441 | 0.345 | 0.212 | 0.029 |
| 34041_at | CCL22 | 0.446 | 0.337 | 0.099 | 0.036 |
| 36445_at | CCL23 | 0.182 | 0.002 | 0.029 | 0.017 |
| 36444_s_at | CCL23 | 0.131 | 0.013 | 0.020 | 0.003 |
| 1924_at | CCNH | 0.043 | 0.106 | 0.070 | 0.020 |
| 39936_at | CCR2 /// | 0.316 | 0.523 | 0.237 | 0.021 |
| | LOC653518 | | | | |
| 35759_at | CCT2 | 0.205 | 0.016 | 0.026 | 0.044 |
| 39767_at | CCT8 | 0.384 | 0.443 | 0.278 | 0.036 |
| 36661_s_at | CD14 | 0.465 | 0.012 | 0.110 | 0.090 |
| 31438_s_at | CD163 | 0.194 | 0.036 | 0.061 | 0.083 |
| 34926_at | CD1A | 0.148 | 0.002 | 0.000 | 0.000 |
| 34927_at | CD1B | 0.405 | 0.003 | 0.005 | 0.011 |
| 37835_at | CD1C | 0.436 | 0.003 | 0.000 | 0.001 |
| 37861_at | CD1E | 0.311 | 0.000 | 0.000 | 0.003 |
| 34699_at | CD2AP | 0.160 | 0.021 | 0.107 | 0.015 |
| 34760_at | CD302 | 0.217 | 0.043 | 0.277 | 0.123 |
| 31870_at | CD37 | 0.352 | 0.097 | 0.046 | 0.008 |
| 31472_s_at | CD44 | 0.217 | 0.015 | 0.127 | 0.148 |
| 1125_s_at | CD44 | 0.136 | 0.037 | 0.271 | 0.171 |
| 1126_s_at | CD44 | 0.235 | 0.048 | 0.111 | 0.199 |
| 38006_at | CD48 | 0.077 | 0.027 | 0.028 | 0.005 |
| 39351_at | CD59 | 0.255 | 0.512 | 0.169 | 0.025 |
| 37536_at | CD83 | 0.080 | 0.113 | 0.212 | 0.027 |
| 505_at | CDC37 | 0.148 | 0.058 | 0.229 | 0.011 |
| 2031_s_at | CDKN1A | 0.017 | 0.009 | 0.018 | 0.003 |
| 36053_at | CDKN2C | 0.514 | 0.048 | 0.278 | 0.126 |
| 36190_at | CDR2 | 0.038 | 0.151 | 0.011 | 0.074 |
| 1052_s_at | CEBPD | 0.017 | 0.016 | 0.046 | 0.018 |
| 32589_at | CHAF1A | 0.373 | 0.031 | 0.042 | 0.044 |
| 33569_at | CLEC10A | 0.419 | 0.040 | 0.007 | 0.002 |
| 40698_at | CLEC2B | 0.095 | 0.106 | 0.055 | 0.020 |
| 40013_at | CLIC2 | 0.475 | 0.009 | 0.005 | 0.001 |
| 39960_at | COQ2 | 0.473 | 0.155 | 0.029 | 0.319 |
| 40427_at | COX17 | 0.018 | 0.090 | 0.099 | 0.017 |
| 39921_at | COX5B | 0.534 | 0.592 | 0.060 | 0.008 |
| 36687_at | COX7B | 0.504 | 0.298 | 0.068 | 0.029 |
| 39692_at | CREB3L2 | 0.328 | 0.585 | 0.018 | 0.027 |
| 39438_at | CREBL2 | 0.326 | 0.068 | 0.002 | 0.169 |
| 33232_at | CRIP1 | 0.534 | 0.418 | 0.046 | 0.127 |
| 40119_at | CRTAP | 0.357 | 0.182 | 0.284 | 0.047 |
| 34223_at | CSF3R | 0.465 | 0.093 | 0.085 | 0.025 |
| 596_s_at | CSF3R | 0.483 | 0.182 | 0.130 | 0.011 |
| 410_s_at | CSNK2B | 0.214 | 0.071 | 0.115 | 0.008 |
| 38112_g_at | CSPG2 | 0.529 | 0.156 | 0.048 | 0.007 |
| 31682_s_at | CSPG2 | 0.545 | 0.399 | 0.213 | 0.020 |
| 39581_at | CSTA | 0.312 | 0.073 | 0.002 | 0.004 |
| 35331_at | CTNNAL1 | 0.054 | 0.002 | 0.000 | 0.001 |
| 40444_s_at | CTNND1 | 0.148 | 0.013 | 0.035 | 0.205 |
| 36566_at | CTNS | 0.007 | 0.012 | 0.028 | 0.003 |
| 133_at | CTSC | 0.061 | 0.002 | 0.000 | 0.000 |
| 239_at | CTSD | 0.445 | 0.309 | 0.037 | 0.002 |
| 38466_at | CTSK | 0.248 | 0.043 | 0.012 | 0.003 |
| 41239_r_at | CTSS | 0.069 | 0.275 | 0.423 | 0.007 |
| 31823_at | CUTL1 | 0.476 | 0.265 | 0.505 | 0.041 |
| 40646_at | CX3CR1 | 0.138 | 0.002 | 0.048 | 0.076 |
| 37187_at | CXCL2 | 0.479 | 0.155 | 0.068 | 0.005 |
| 649_s_at | CXCR4 | 0.043 | 0.045 | 0.095 | 0.023 |
| 40296_at | CXorf9 | 0.482 | 0.231 | 0.247 | 0.036 |
| 999_at | CYP27A1 | 0.424 | 0.344 | 0.212 | 0.027 |
| 33389_at | CYP51A1 | 0.265 | 0.041 | 0.148 | 0.082 |
| 33753_at | DAAM1 | 0.198 | 0.467 | 0.114 | 0.007 |
| 1243_at | DDB2 | 0.178 | 0.092 | 0.027 | 0.035 |
| 38104_at | DECR1 | 0.427 | 0.515 | 0.089 | 0.047 |
| 41734_at | DENND3 | 0.273 | 0.250 | 0.099 | 0.023 |
| 41637_at | DEXI | 0.323 | 0.630 | 0.148 | 0.033 |
| 41872_at | DFNA5 | 0.238 | 0.063 | 0.076 | 0.005 |
| 39044_s_at | DGKD | 0.188 | 0.125 | 0.039 | 0.208 |
| 39814_s_at | DHRS7 | 0.278 | 0.042 | 0.079 | 0.008 |
| 41402_at | DKFZP564O0823 | 0.229 | 0.015 | 0.134 | 0.020 |
| 41716_at | DMXL2 | 0.151 | 0.103 | 0.065 | 0.016 |
| 35799_at | DNAJB9 | 0.045 | 0.539 | 0.161 | 0.445 |
| 40607_at | DPYSL2 | 0.358 | 0.043 | 0.035 | 0.015 |
| 32168_s_at | DSCR1 | 0.042 | 0.432 | 0.084 | 0.008 |
| 38555_at | DUSP10 | 0.017 | 0.003 | 0.031 | 0.010 |
| 41193_at | DUSP6 | 0.426 | 0.015 | 0.016 | 0.002 |
| 36921_at | DYNLT3 | 0.181 | 0.025 | 0.225 | 0.067 |
| 37016_at | ECHS1 | 0.556 | 0.276 | 0.225 | 0.020 |
| 40886_at | EEF1A1 /// | 0.329 | 0.390 | 0.484 | 0.037 |
| | APOLD1 /// | | | | |
| | LOC440595 | | | | |
| 37863_at | EGR2 | 0.273 | 0.025 | 0.000 | 0.005 |
| 33351_at | EIF1B | 0.170 | 0.563 | 0.015 | 0.177 |
| 34302_at | EIF3S4 | 0.258 | 0.242 | 0.220 | 0.029 |
| 35323_at | EIF3S9 | 0.134 | 0.118 | 0.402 | 0.025 |
| 37527_at | ELK3 | 0.038 | 0.568 | 0.304 | 0.351 |
| 40606_at | ELL2 | 0.093 | 0.439 | 0.406 | 0.017 |
| 39542_at | ENC1 | 0.452 | 0.229 | 0.018 | 0.100 |
| 32562_at | ENG | 0.250 | 0.352 | 0.123 | 0.037 |
| 41123_s_at | ENPP2 | 0.209 | 0.016 | 0.058 | 0.391 |
| 41124_r_at | ENPP2 | 0.255 | 0.057 | 0.048 | 0.478 |
| 32585_at | EPB41L2 | 0.510 | 0.014 | 0.040 | 0.013 |
| 902_at | EPHB2 | 0.200 | 0.038 | 0.085 | 0.177 |
| 41678_at | EPHB2 | 0.410 | 0.006 | 0.036 | 0.091 |
| 37731_at | EPS15 | 0.531 | 0.399 | 0.020 | 0.002 |
| 38158_at | ESPL1 | 0.132 | 0.074 | 0.212 | 0.014 |
| 38739_at | ETS2 | 0.488 | 0.559 | 0.134 | 0.036 |
| 32259_at | EZH1 | 0.354 | 0.327 | 0.026 | 0.166 |
| 40143_at | FAM53B | 0.043 | 0.260 | 0.387 | 0.520 |
| 32209_at | FAM89B | 0.512 | 0.652 | 0.586 | 0.022 |
| 38318_at | FAM8A1 | 0.166 | 0.330 | 0.106 | 0.046 |
| 36495_at | FBP1 | 0.387 | 0.615 | 0.409 | 0.027 |
| 34959_at | FCER2 | 0.166 | 0.000 | 0.000 | 0.000 |
| 34960_g_at | FCER2 | 0.078 | 0.001 | 0.000 | 0.001 |
| 37688_f_at | FCGR2A | 0.522 | 0.376 | 0.050 | 0.001 |
| 37689_s_at | FCGR2A | 0.349 | 0.661 | 0.028 | 0.014 |
| 37687_i_at | FCGR2B | 0.363 | 0.446 | 0.212 | 0.008 |
| 37200_at | FCGR3A | 0.160 | 0.015 | 0.004 | 0.008 |
| 31499_s_at | FCGR3B | 0.185 | 0.008 | 0.079 | 0.045 |
| 39593_at | FGL2 | 0.317 | 0.199 | 0.183 | 0.045 |
| 39591_s_at | FGL2 | 0.346 | 0.130 | 0.191 | 0.024 |
| 32546_at | FH | 0.542 | 0.140 | 0.023 | 0.037 |
| 880_at | FKBP1A | 0.148 | 0.003 | 0.002 | 0.008 |
| 41425_at | FLI1 | 0.122 | 0.144 | 0.188 | 0.032 |
| 41814_at | FUCA1 | 0.515 | 0.023 | 0.036 | 0.040 |
| 35338_at | FURIN | 0.283 | 0.184 | 0.069 | 0.008 |
| 34716_at | FUSIP1 /// | 0.031 | 0.525 | 0.333 | 0.302 |
| | LOC642558 | | | | |
| 41819_at | FYB | 0.168 | 0.037 | 0.065 | 0.006 |
| 38326_at | G0S2 | 0.212 | 0.021 | 0.170 | 0.096 |
| 33936_at | GALC | 0.257 | 0.024 | 0.105 | 0.064 |
| 1598_g_at | GAS6 | 0.175 | 0.017 | 0.040 | 0.016 |
| 1597_at | GAS6 | 0.060 | 0.002 | 0.005 | 0.067 |
| 37658_at | GAS6 | 0.262 | 0.015 | 0.028 | 0.009 |
| 33387_at | GAS7 | 0.046 | 0.018 | 0.045 | 0.040 |
| 36596_r_at | GATM | 0.299 | 0.245 | 0.253 | 0.033 |
| 32643_at | GBE1 | 0.217 | 0.660 | 0.432 | 0.025 |
| 32700_at | GBP2 | 0.570 | 0.035 | 0.204 | 0.139 |
| 37944_at | GCH1 | 0.415 | 0.531 | 0.021 | 0.064 |
| 38237_at | GGTLA1 | 0.280 | 0.010 | 0.003 | 0.016 |
| 34311_at | GLRX | 0.034 | 0.219 | 0.217 | 0.087 |
| 40522_at | GLUL | 0.018 | 0.092 | 0.013 | 0.012 |
| 31812_at | GMPR | 0.271 | 0.061 | 0.031 | 0.264 |
| 35272_at | GNG5 | 0.339 | 0.623 | 0.067 | 0.016 |
| 38379_at | GPNMB | 0.297 | 0.054 | 0.048 | 0.008 |
| 31700_at | GPR35 | 0.169 | 0.042 | 0.068 | 0.027 |
| 34930_at | GPR65 | 0.235 | 0.284 | 0.189 | 0.023 |
| 40994_at | GRK5 | 0.485 | 0.270 | 0.055 | 0.024 |
| 33932_at | GSPT1 | 0.207 | 0.260 | 0.018 | 0.055 |
| 869_at | GTF2A2 | 0.326 | 0.321 | 0.048 | 0.022 |
| 35821_at | HDAC3 | 0.148 | 0.026 | 0.083 | 0.088 |
| 40121_at | HIP2 | 0.017 | 0.616 | 0.459 | 0.466 |
| 32980_f_at | HIST1H2BC | 0.540 | 0.558 | 0.149 | 0.009 |
| 31522_f_at | HIST1H2BF | 0.224 | 0.641 | 0.273 | 0.047 |
| 31524_f_at | HIST1H2BI | 0.410 | 0.412 | 0.227 | 0.046 |
| 35576_f_at | HIST1H2BL | 0.500 | 0.525 | 0.208 | 0.038 |
| 31528_f_at | HIST1H2BM | 0.527 | 0.367 | 0.142 | 0.043 |
| 36347_f_at | HIST1H2BN | 0.570 | 0.600 | 0.229 | 0.031 |
| 40964_at | HK2 | 0.044 | 0.016 | 0.048 | 0.011 |
| 37604_at | HNMT | 0.072 | 0.475 | 0.223 | 0.021 |
| 38292_at | HOMER2 | 0.138 | 0.083 | 0.008 | 0.014 |
| 38233_at | HOMER3 | 0.146 | 0.003 | 0.037 | 0.074 |
| 36030_at | HOM-TES- | 0.069 | 0.026 | 0.068 | 0.071 |
| | 103 | | | | |
| 35702_at | HSD11B1 | 0.395 | 0.028 | 0.066 | 0.046 |
| 32316_s_at | HSP90AA1 | 0.108 | 0.105 | 0.005 | 0.016 |
| 1161_at | HSP90AB1 | 0.088 | 0.083 | 0.010 | 0.198 |
| 33984_at | HSP90AB1 | 0.209 | 0.118 | 0.029 | 0.015 |
| 31692_at | HSPA1A | 0.388 | 0.456 | 0.049 | 0.192 |
| 35965_at | HSPA6 | 0.220 | 0.002 | 0.097 | 0.023 |
| 117_at | HSPA6 /// | 0.438 | 0.053 | 0.005 | 0.091 |
| | LOC652878 | | | | |
| 40637_at | HSPA8 | 0.448 | 0.606 | 0.002 | 0.306 |
| 36785_at | HSPB1 | 0.330 | 0.070 | 0.025 | 0.011 |
| 41259_at | HSPC111 | 0.148 | 0.057 | 0.044 | 0.006 |
| 37720_at | HSPD1 | 0.170 | 0.043 | 0.085 | 0.008 |
| 39353_at | HSPE1 | 0.288 | 0.399 | 0.010 | 0.067 |
| 719_g_at | HTRA1 | 0.168 | 0.004 | 0.018 | 0.009 |
| 718_at | HTRA1 | 0.183 | 0.015 | 0.000 | 0.020 |
| 36564_at | IBRDC3 | 0.060 | 0.043 | 0.015 | 0.038 |
| 38454_g_at | ICAM2 | 0.403 | 0.517 | 0.099 | 0.022 |
| 37043_at | ID3 | 0.050 | 0.099 | 0.110 | 0.020 |
| 36927_at | IFI44L | 0.354 | 0.162 | 0.117 | 0.026 |
| 676_g_at | IFITM1 /// | 0.490 | 0.418 | 0.037 | 0.009 |
| | IFITM3 /// | | | | |
| | IFITM2 | | | | |
| 41745_at | IFITM3 | 0.394 | 0.466 | 0.088 | 0.014 |
| 1038_s_at | IFNGR1 | 0.017 | 0.050 | 0.067 | 0.032 |
| 34946_at | IGSF6 | 0.315 | 0.358 | 0.040 | 0.119 |
| 1061_at | IL10RA | 0.030 | 0.076 | 0.119 | 0.012 |
| 359_at | IL13RA1 | 0.204 | 0.088 | 0.235 | 0.025 |
| 1165_at | IL18 | 0.549 | 0.016 | 0.182 | 0.024 |
| 39402_at | IL1B | 0.236 | 0.614 | 0.323 | 0.044 |
| 1368_at | IL1R1 | 0.108 | 0.002 | 0.000 | 0.000 |
| 998_s_at | IL1R2 | 0.231 | 0.005 | 0.020 | 0.027 |
| 37603_at | IL1RN | 0.046 | 0.002 | 0.067 | 0.044 |
| 37844_at | IL27RA | 0.383 | 0.061 | 0.015 | 0.001 |
| 37843_i_at | IL27RA | 0.488 | 0.090 | 0.021 | 0.025 |
| 1185_at | IL3RA | 0.017 | 0.001 | 0.064 | 0.092 |
| 1369_s_at | IL8 | 0.529 | 0.536 | 0.367 | 0.049 |
| 37276_at | IQGAP2 | 0.217 | 0.060 | 0.285 | 0.021 |
| 37625_at | IRF4 | 0.007 | 0.269 | 0.014 | 0.085 |
| 35731_at | ITGA4 | 0.360 | 0.093 | 0.048 | 0.065 |
| 38533_s_at | ITGAM | 0.187 | 0.015 | 0.073 | 0.022 |
| 36709_at | ITGAX | 0.176 | 0.234 | 0.086 | 0.035 |
| 41300_s_at | ITM2B | 0.571 | 0.110 | 0.016 | 0.006 |
| 32778_at | ITPR1 | 0.210 | 0.077 | 0.006 | 0.018 |
| 755_at | ITPR1 | 0.316 | 0.017 | 0.055 | 0.016 |
| 33178_at | JAG1 | 0.003 | 0.439 | 0.361 | 0.312 |
| 35414_s_at | JAG1 | 0.003 | 0.017 | 0.090 | 0.001 |
| 34786_at | JMJD1A | 0.043 | 0.497 | 0.348 | 0.376 |
| 38972_at | KCTD12 | 0.033 | 0.026 | 0.119 | 0.017 |
| 39783_at | KIAA0100 | 0.189 | 0.167 | 0.068 | 0.001 |
| 35744_at | KIAA0141 | 0.404 | 0.598 | 0.012 | 0.496 |
| 31863_at | KIAA0179 | 0.295 | 0.151 | 0.006 | 0.002 |
| 38735_at | KIAA0513 | 0.019 | 0.370 | 0.114 | 0.268 |
| 35252_at | KIAA0528 | 0.488 | 0.456 | 0.447 | 0.022 |
| 39559_at | KMO | 0.314 | 0.218 | 0.031 | 0.131 |
| 180_at | LENG4 | 0.521 | 0.346 | 0.269 | 0.048 |
| 37542_at | LHFPL2 | 0.272 | 0.015 | 0.035 | 0.005 |
| 38618_at | LIMK2 /// | 0.326 | 0.283 | 0.164 | 0.015 |
| | PPP1R14BP1 | | | | |
| 39232_at | LIMS1 | 0.559 | 0.198 | 0.023 | 0.055 |
| 38745_at | LIPA | 0.522 | 0.012 | 0.018 | 0.061 |
| 31936_s_at | LKAP | 0.017 | 0.189 | 0.361 | 0.365 |
| 32195_at | LOC339287 | 0.284 | 0.048 | 0.197 | 0.521 |
| 39879_s_at | LOC388397 | 0.481 | 0.400 | 0.275 | 0.027 |
| 33866_at | LOC643634 | 0.496 | 0.370 | 0.015 | 0.041 |
| 39937_at | LOC653518 | 0.303 | 0.626 | 0.349 | 0.036 |
| 38775_at | LRP1 | 0.514 | 0.184 | 0.020 | 0.117 |
| 41320_s_at | LRRFIP1 | 0.535 | 0.270 | 0.004 | 0.039 |
| 36493_at | LSP1 /// | 0.530 | 0.461 | 0.129 | 0.035 |
| | LOC649377 | | | | |
| 38081_at | LTA4H | 0.294 | 0.042 | 0.032 | 0.002 |
| 35869_at | LY86 | 0.467 | 0.169 | 0.175 | 0.020 |
| 41505_r_at | MAF | 0.020 | 0.200 | 0.158 | 0.413 |
| 36711_at | MAFF | 0.039 | 0.015 | 0.015 | 0.022 |
| 37472_at | MANBA | 0.080 | 0.002 | 0.037 | 0.052 |
| 41772_at | MAOA | 0.005 | 0.002 | 0.010 | 0.001 |
| 41771_g_at | MAOA | 0.007 | 0.000 | 0.006 | 0.000 |
| 41770_at | MAOA | 0.004 | 0.000 | 0.004 | 0.001 |
| 976_s_at | MAPK1 | 0.111 | 0.332 | 0.023 | 0.088 |
| 33223_at | MAST3 | 0.093 | 0.334 | 0.035 | 0.490 |
| 32571_at | MAT2A | 0.078 | 0.091 | 0.097 | 0.024 |
| 34386_at | MBD4 | 0.039 | 0.326 | 0.534 | 0.088 |
| 36608_at | MDH1 | 0.528 | 0.004 | 0.001 | 0.002 |
| 35629_at | MKL1 | 0.147 | 0.008 | 0.291 | 0.015 |
| 32207_at | MPP1 | 0.131 | 0.018 | 0.023 | 0.073 |
| 36908_at | MRC1 /// | 0.003 | 0.002 | 0.001 | 0.056 |
| | MRC1L1 | | | | |
| 39812_at | MRPL12 | 0.534 | 0.019 | 0.212 | 0.043 |
| 35992_at | MSC | 0.171 | 0.102 | 0.079 | 0.021 |
| 674_g_at | MTHFD1 | 0.348 | 0.534 | 0.111 | 0.023 |
| 40074_at | MTHFD2 | 0.534 | 0.226 | 0.023 | 0.008 |
| 879_at | MX2 | 0.465 | 0.050 | 0.078 | 0.041 |
| 1973_s_at | MYC | 0.160 | 0.038 | 0.040 | 0.047 |
| 38369_at | MYD88 | 0.099 | 0.043 | 0.191 | 0.535 |
| 32069_at | N4BP1 | 0.104 | 0.305 | 0.023 | 0.040 |
| 41249_at | NADK | 0.046 | 0.118 | 0.166 | 0.168 |
| 36607_at | NAGA | 0.305 | 0.234 | 0.131 | 0.015 |
| 38187_at | NAT1 | 0.525 | 0.203 | 0.010 | 0.319 |
| 34279_at | NBPF14 /// | 0.359 | 0.632 | 0.288 | 0.044 |
| | NBPF1 /// | | | | |
| | KIAA1245 /// | | | | |
| | NBPF11 /// | | | | |
| | NBPF15 /// | | | | |
| | NBPF20 /// | | | | |
| | NBPF9 /// | | | | |
| | NBPF10 /// | | | | |
| | NBPF12 /// | | | | |
| | NBPF8 /// | | | | |
| | NBPF16 | | | | |
| 39174_at | NCOA4 | 0.216 | 0.187 | 0.099 | 0.011 |
| 39358_at | NCOR2 | 0.543 | 0.051 | 0.291 | 0.034 |
| 38257_at | NDUFS8 | 0.321 | 0.308 | 0.260 | 0.046 |
| 34893_at | NDUFV2 | 0.030 | 0.063 | 0.157 | 0.170 |
| 37544_at | NFIL3 | 0.357 | 0.037 | 0.037 | 0.029 |
| 35366_at | NID1 | 0.414 | 0.160 | 0.170 | 0.015 |
| 39073_at | NME1 | 0.278 | 0.111 | 0.020 | 0.020 |
| 1985_s_at | NME1 | 0.209 | 0.021 | 0.032 | 0.016 |
| 1979_s_at | NOL1 | 0.560 | 0.025 | 0.044 | 0.007 |
| 32719_at | NRG1 | 0.388 | 0.461 | 0.029 | 0.047 |
| 40088_at | NRIP1 | 0.158 | 0.089 | 0.023 | 0.047 |
| 32644_at | NUP188 | 0.175 | 0.099 | 0.235 | 0.014 |
| 40768_s_at | NUP214 | 0.003 | 0.148 | 0.003 | 0.004 |
| 34491_at | OASL | 0.405 | 0.416 | 0.313 | 0.016 |
| 36134_at | OLFM1 | 0.121 | 0.051 | 0.023 | 0.022 |
| 38855_s_at | OLFM1 | 0.272 | 0.276 | 0.040 | 0.017 |
| 36007_at | OLFML2B | 0.197 | 0.004 | 0.099 | 0.024 |
| 36689_at | OSBPL1A | 0.297 | 0.540 | 0.106 | 0.031 |
| 35674_at | PADI2 | 0.556 | 0.073 | 0.018 | 0.012 |
| 39056_at | PAICS | 0.537 | 0.094 | 0.184 | 0.030 |
| 1560_g_at | PAK2 | 0.455 | 0.025 | 0.182 | 0.093 |
| 41191_at | PALLD | 0.017 | 0.017 | 0.012 | 0.006 |
| 38465_at | PAM | 0.101 | 0.024 | 0.032 | 0.005 |
| 34352_at | PCBD1 | 0.217 | 0.063 | 0.080 | 0.008 |
| 37188_at | PCK2 | 0.107 | 0.030 | 0.073 | 0.201 |
| 1884_s_at | PCNA | 0.031 | 0.614 | 0.335 | 0.552 |
| 32212_at | PDCD8 | 0.540 | 0.088 | 0.118 | 0.034 |
| 746_at | PDE3B | 0.431 | 0.365 | 0.202 | 0.028 |
| 36092_at | PDE4DIP | 0.223 | 0.450 | 0.247 | 0.036 |
| 35714_at | PDXK | 0.304 | 0.000 | 0.144 | 0.003 |
| 37397_at | PECAM1 | 0.351 | 0.036 | 0.143 | 0.106 |
| 32455_s_at | PELP1 | 0.335 | 0.025 | 0.196 | 0.326 |
| 39175_at | PFKP | 0.055 | 0.019 | 0.014 | 0.002 |
| 36502_at | PFTK1 | 0.350 | 0.601 | 0.132 | 0.022 |
| 32739_at | PGM3 | 0.282 | 0.507 | 0.040 | 0.214 |
| 33333_at | PIP3-E | 0.146 | 0.239 | 0.232 | 0.009 |
| 34839_at | PITRM1 | 0.126 | 0.058 | 0.023 | 0.002 |
| 33707_at | PLA2G4C | 0.341 | 0.305 | 0.308 | 0.047 |
| 36943_r_at | PLAGL1 | 0.175 | 0.590 | 0.501 | 0.035 |
| 37310_at | PLAU | 0.044 | 0.236 | 0.269 | 0.394 |
| 32775_r_at | PLSCR1 | 0.571 | 0.167 | 0.110 | 0.014 |
| 32193_at | PLXNC1 | 0.169 | 0.024 | 0.088 | 0.012 |
| 38653_at | PMP22 | 0.384 | 0.012 | 0.009 | 0.008 |
| 1696_at | POLB | 0.201 | 0.015 | 0.073 | 0.012 |
| 858_at | POR | 0.346 | 0.013 | 0.303 | 0.277 |
| 37104_at | PPARG | 0.014 | 0.159 | 0.123 | 0.032 |
| 41709_at | PPFIBP2 | 0.176 | 0.025 | 0.123 | 0.033 |
| 37384_at | PPM1F | 0.303 | 0.329 | 0.133 | 0.022 |
| 33358_at | PPM1H | 0.288 | 0.038 | 0.095 | 0.017 |
| 41540_at | PPP1R7 | 0.440 | 0.143 | 0.052 | 0.001 |
| 1336_s_at | PRKCB1 | 0.276 | 0.097 | 0.088 | 0.005 |
| 1217_g_at | PRKCB1 | 0.267 | 0.020 | 0.353 | 0.036 |
| 160029_at | PRKCB1 | 0.378 | 0.155 | 0.133 | 0.011 |
| 37969_at | PTGS1 | 0.543 | 0.323 | 0.267 | 0.024 |
| 33804_at | PTK2B | 0.258 | 0.015 | 0.102 | 0.022 |
| 35342_at | PTPLB | 0.408 | 0.493 | 0.241 | 0.008 |
| 36808_at | PTPN22 | 0.172 | 0.080 | 0.048 | 0.012 |
| 39672_at | PTPN7 | 0.398 | 0.276 | 0.068 | 0.043 |
| 40519_at | PTPRC | 0.201 | 0.231 | 0.119 | 0.038 |
| 32916_at | PTPRE | 0.030 | 0.002 | 0.000 | 0.000 |
| 32199_at | PTPRO | 0.286 | 0.276 | 0.186 | 0.046 |
| 1190_at | PTPRO | 0.336 | 0.200 | 0.095 | 0.026 |
| 35966_at | QPCT | 0.017 | 0.007 | 0.006 | 0.002 |
| 37978_at | QPRT | 0.520 | 0.113 | 0.079 | 0.003 |
| 1257_s_at | QSCN6 | 0.072 | 0.001 | 0.005 | 0.002 |
| 809_at | RAB27A | 0.319 | 0.256 | 0.086 | 0.012 |
| 1202_g_at | RAB33A | 0.098 | 0.043 | 0.146 | 0.024 |
| 35340_at | RAB8A | 0.377 | 0.004 | 0.014 | 0.018 |
| 35339_at | RAB8A | 0.351 | 0.026 | 0.023 | 0.024 |
| 35289_at | RABGAP1 | 0.219 | 0.019 | 0.055 | 0.003 |
| 34445_at | RABGAP1L | 0.205 | 0.160 | 0.113 | 0.031 |
| 37703_at | RABGGTB | 0.354 | 0.251 | 0.095 | 0.019 |
| 1874_at | RAD23B | 0.523 | 0.219 | 0.212 | 0.048 |
| 32593_at | RAFTLIN | 0.275 | 0.050 | 0.089 | 0.065 |
| 35668_at | RAMP1 | 0.225 | 0.269 | 0.075 | 0.007 |
| 41342_at | RANBP1 | 0.322 | 0.615 | 0.000 | 0.437 |
| 34745_at | RAPGEF2 | 0.135 | 0.028 | 0.090 | 0.071 |
| 32026_s_at | RAPGEF2 | 0.101 | 0.024 | 0.108 | 0.035 |
| 1675_at | RASA1 | 0.048 | 0.167 | 0.267 | 0.318 |
| 36935_at | RASA1 | 0.044 | 0.099 | 0.191 | 0.404 |
| 37598_at | RASSF2 | 0.030 | 0.352 | 0.562 | 0.352 |
| 34187_at | RBMS2 | 0.340 | 0.444 | 0.469 | 0.027 |
| 40818_at | RBPSUH | 0.286 | 0.015 | 0.068 | 0.001 |
| 35193_at | RCBTB2 | 0.110 | 0.025 | 0.149 | 0.063 |
| 41172_at | RDH11 | 0.026 | 0.271 | 0.102 | 0.510 |
| 38908_s_at | REV3L | 0.153 | 0.028 | 0.025 | 0.024 |
| 37701_at | RGS2 | 0.014 | 0.030 | 0.109 | 0.034 |
| 36550_at | RIN2 | 0.207 | 0.003 | 0.078 | 0.039 |
| 32664_at | RNASE4 | 0.352 | 0.151 | 0.035 | 0.015 |
| 35777_at | RNF4 | 0.422 | 0.135 | 0.214 | 0.007 |
| 36187_at | RNH1 | 0.104 | 0.059 | 0.034 | 0.001 |
| 41296_s_at | RPS24 | 0.541 | 0.172 | 0.060 | 0.025 |
| 33325_at | RPS6KA2 | 0.125 | 0.074 | 0.018 | 0.230 |
| 32544_s_at | RSU1 | 0.318 | 0.619 | 0.169 | 0.028 |
| 106_at | RUNX3 | 0.341 | 0.118 | 0.066 | 0.002 |
| 37732_at | RYBP | 0.212 | 0.190 | 0.012 | 0.011 |
| 539_at | RYK | 0.311 | 0.024 | 0.032 | 0.057 |
| 41096_at | S100A8 | 0.364 | 0.071 | 0.014 | 0.040 |
| 41471_at | S100A9 | 0.510 | 0.390 | 0.030 | 0.025 |
| 34304_s_at | SAT | 0.148 | 0.106 | 0.139 | 0.040 |
| 41200_at | SCARB1 | 0.206 | 0.088 | 0.014 | 0.012 |
| 36192_at | SCRN1 | 0.453 | 0.131 | 0.036 | 0.044 |
| 39757_at | SDC2 | 0.372 | 0.025 | 0.070 | 0.008 |
| 40390_at | SDS | 0.056 | 0.098 | 0.182 | 0.047 |
| 41597_s_at | SEC22B | 0.178 | 0.294 | 0.198 | 0.014 |
| 245_at | SELL | 0.534 | 0.478 | 0.103 | 0.039 |
| 34363_at | SEPP1 | 0.326 | 0.020 | 0.021 | 0.050 |
| 37185_at | SERPINB2 | 0.496 | 0.197 | 0.086 | 0.036 |
| 34438_at | SERPINB9 | 0.102 | 0.157 | 0.043 | 0.482 |
| 40856_at | SERPINF1 | 0.195 | 0.022 | 0.080 | 0.014 |
| 40638_at | SFPQ | 0.221 | 0.368 | 0.048 | 0.057 |
| 40457_at | SFRS3 | 0.517 | 0.563 | 0.021 | 0.256 |
| 973_at | SGK | 0.152 | 0.013 | 0.003 | 0.023 |
| 38968_at | SH3BP5 | 0.135 | 0.173 | 0.055 | 0.040 |
| 1427_g_at | SLA | 0.043 | 0.016 | 0.002 | 0.005 |
| 1426_at | SLA | 0.007 | 0.002 | 0.001 | 0.001 |
| 1138_at | SLC20A1 | 0.028 | 0.000 | 0.001 | 0.036 |
| 38122_at | SLC23A2 | 0.095 | 0.197 | 0.066 | 0.015 |
| 37740_r_at | SLC25A5 | 0.102 | 0.414 | 0.047 | 0.357 |
| 36979_at | SLC2A3 | 0.022 | 0.221 | 0.342 | 0.344 |
| 34749_at | SLC31A2 | 0.031 | 0.153 | 0.287 | 0.355 |
| 37895_at | SLC35A1 | 0.169 | 0.030 | 0.070 | 0.017 |
| 1798_at | SLC39A6 | 0.431 | 0.401 | 0.036 | 0.147 |
| 33731_at | SLC7A7 | 0.111 | 0.035 | 0.014 | 0.037 |
| 40810_at | SMARCC1 | 0.204 | 0.529 | 0.128 | 0.032 |
| 39950_at | SMPDL3A | 0.017 | 0.453 | 0.215 | 0.029 |
| 33354_at | SMURF2 | 0.143 | 0.522 | 0.573 | 0.035 |
| 40842_at | SNRPA | 0.501 | 0.170 | 0.237 | 0.007 |
| 40605_at | SNX4 | 0.258 | 0.586 | 0.023 | 0.316 |
| 41592_at | SOCS1 | 0.010 | 0.002 | 0.005 | 0.020 |
| 32140_at | SORL1 | 0.385 | 0.023 | 0.011 | 0.003 |
| 41573_at | SP3 | 0.140 | 0.236 | 0.159 | 0.015 |
| 671_at | SPARC | 0.565 | 0.552 | 0.122 | 0.006 |
| 1685_at | SPHAR | 0.358 | 0.048 | 0.513 | 0.446 |
| 33448_at | SPINT1 | 0.446 | 0.030 | 0.012 | 0.006 |
| 34348_at | SPINT2 | 0.043 | 0.002 | 0.000 | 0.000 |
| 36798_g_at | SPN | 0.061 | 0.002 | 0.054 | 0.012 |
| 34342_s_at | SPP1 | 0.557 | 0.513 | 0.201 | 0.020 |
| 2092_s_at | SPP1 | 0.571 | 0.559 | 0.166 | 0.023 |
| 32135_at | SREBF1 | 0.076 | 0.239 | 0.124 | 0.039 |
| 40109_at | SRF | 0.175 | 0.473 | 0.183 | 0.016 |
| 35231_at | SRP19 | 0.043 | 0.334 | 0.126 | 0.062 |
| 1640_at | ST13 | 0.311 | 0.160 | 0.014 | 0.023 |
| 39298_at | ST3GAL6 | 0.000 | 0.002 | 0.006 | 0.020 |
| 38487_at | STAB1 | 0.483 | 0.224 | 0.014 | 0.068 |
| 38525_at | STAM2 | 0.211 | 0.163 | 0.031 | 0.350 |
| 41295_at | STARD7 | 0.297 | 0.013 | 0.018 | 0.012 |
| AFFX- | STAT1 | 0.275 | 0.021 | 0.195 | 0.104 |
| HUMISGF3A/ | | | | | |
| M | | | | | |
| 97935_3_at | | | | | |
| 33339_g_at | STAT1 | 0.465 | 0.048 | 0.095 | 0.113 |
| 39708_at | STAT3 | 0.291 | 0.637 | 0.551 | 0.022 |
| 40473_at | STK24 | 0.022 | 0.170 | 0.368 | 0.240 |
| 32182_at | STK38L | 0.160 | 0.048 | 0.204 | 0.348 |
| 41663_at | STX6 | 0.030 | 0.002 | 0.025 | 0.071 |
| 41034_s_at | SULT2B1 | 0.456 | 0.332 | 0.492 | 0.023 |
| 31869_at | SWAP70 | 0.018 | 0.088 | 0.133 | 0.024 |
| 34885_at | SYNGR2 | 0.017 | 0.079 | 0.031 | 0.002 |
| 36532_at | SYNJ2 | 0.020 | 0.398 | 0.036 | 0.119 |
| 34966_at | T | 0.465 | 0.433 | 0.021 | 0.173 |
| 39416_at | TAX1BP3 | 0.540 | 0.361 | 0.046 | 0.012 |
| 38317_at | TCEAL1 | 0.504 | 0.612 | 0.243 | 0.020 |
| 40865_at | TDG | 0.352 | 0.453 | 0.229 | 0.037 |
| 160025_at | TGFA | 0.046 | 0.051 | 0.167 | 0.266 |
| 38805_at | TGIF | 0.072 | 0.016 | 0.032 | 0.216 |
| 38404_at | TGM2 | 0.041 | 0.000 | 0.014 | 0.010 |
| 32829_at | TIMM17A | 0.054 | 0.015 | 0.014 | 0.079 |
| 39411_at | TIPARP | 0.501 | 0.318 | 0.186 | 0.048 |
| 38364_at | TLE4 | 0.017 | 0.335 | 0.382 | 0.150 |
| 40310_at | TLR2 | 0.413 | 0.110 | 0.139 | 0.029 |
| 34473_at | TLR5 | 0.170 | 0.168 | 0.070 | 0.008 |
| 32116_at | TMC6 | 0.208 | 0.019 | 0.099 | 0.048 |
| 36950_at | TMED9 | 0.194 | 0.044 | 0.490 | 0.044 |
| 39424_at | TNFRSF14 | 0.146 | 0.017 | 0.015 | 0.075 |
| 1583_at | TNFRSF1B | 0.014 | 0.015 | 0.023 | 0.020 |
| 33813_at | TNFRSF1B | 0.043 | 0.068 | 0.040 | 0.059 |
| 1715_at | TNFSF10 | 0.245 | 0.061 | 0.099 | 0.028 |
| 1030_s_at | TOP1 | 0.038 | 0.325 | 0.021 | 0.070 |
| 31680_at | TOP1P2 | 0.033 | 0.298 | 0.132 | 0.039 |
| 36139_at | TRAF3IP2 | 0.574 | 0.130 | 0.429 | 0.016 |
| 35238_at | TRAF5 | 0.453 | 0.175 | 0.501 | 0.050 |
| 1468_at | TRAP1 | 0.427 | 0.070 | 0.265 | 0.016 |
| 41468_at | TRGC2 /// | 0.021 | 0.198 | 0.501 | 0.275 |
| | TRGV2 /// | | | | |
| | TRGV9 /// | | | | |
| | TARP /// | | | | |
| | LOC642083 | | | | |
| 36825_at | TRIM22 | 0.466 | 0.129 | 0.139 | 0.014 |
| 39032_at | TSC22D1 | 0.153 | 0.418 | 0.259 | 0.049 |
| 36629_at | TSC22D3 | 0.139 | 0.192 | 0.146 | 0.030 |
| 32730_at | TSPYL5 | 0.135 | 0.072 | 0.025 | 0.060 |
| 34825_at | TTRAP | 0.168 | 0.403 | 0.166 | 0.022 |
| 38350_f_at | TUBA2 | 0.148 | 0.173 | 0.018 | 0.003 |
| 40567_at | TUBA3 | 0.160 | 0.199 | 0.018 | 0.001 |
| 151_s_at | TUBB | 0.297 | 0.530 | 0.236 | 0.007 |
| 429_f_at | TUBB2A /// | 0.284 | 0.073 | 0.067 | 0.007 |
| | TUBB4 /// | | | | |
| | TUBB2B | | | | |
| 33678_i_at | TUBB2C | 0.187 | 0.037 | 0.036 | 0.002 |
| 33679_f_at | TUBB2C | 0.153 | 0.029 | 0.061 | 0.005 |
| 471_f_at | TUBB3 | 0.228 | 0.317 | 0.029 | 0.014 |
| 38089_at | UBAP2L | 0.174 | 0.058 | 0.041 | 0.003 |
| 39040_at | UBE2J1 | 0.155 | 0.004 | 0.042 | 0.073 |
| 223_at | UBE2L3 | 0.275 | 0.091 | 0.008 | 0.029 |
| 40505_at | UBE2L6 | 0.322 | 0.439 | 0.085 | 0.022 |
| 40839_at | UBL3 | 0.022 | 0.487 | 0.216 | 0.007 |
| 39442_at | UNC50 | 0.022 | 0.453 | 0.409 | 0.571 |
| 283_at | UQCRC1 | 0.213 | 0.043 | 0.086 | 0.006 |
| 41859_at | UST | 0.504 | 0.036 | 0.295 | 0.154 |
| 34481_at | VAV1 | 0.007 | 0.406 | 0.095 | 0.328 |
| 36601_at | VCL | 0.360 | 0.248 | 0.197 | 0.018 |
| 31608_g_at | VDAC1 | 0.423 | 0.086 | 0.096 | 0.014 |
| 40198_at | VDAC1 | 0.358 | 0.092 | 0.044 | 0.004 |
| 1388_g_at | VDR | 0.054 | 0.030 | 0.027 | 0.005 |
| 1410_at | VDR | 0.195 | 0.050 | 0.015 | 0.023 |
| 34498_at | VNN2 | 0.343 | 0.028 | 0.021 | 0.023 |
| 1669_at | WNT5A | 0.183 | 0.043 | 0.015 | 0.007 |
| 31862_at | WNT5A | 0.078 | 0.086 | 0.043 | 0.009 |
| 40167_s_at | WSB2 | 0.549 | 0.276 | 0.024 | 0.126 |
| 783_at | WWP1 | 0.077 | 0.015 | 0.014 | 0.020 |
| 784_g_at | WWP1 | 0.137 | 0.044 | 0.035 | 0.057 |
| 39755_at | XBP1 | 0.017 | 0.002 | 0.387 | 0.376 |
| 39756_g_at | XBP1 | 0.038 | 0.073 | 0.222 | 0.198 |
| 41669_at | ZCCHC11 | 0.250 | 0.419 | 0.168 | 0.020 |
| 35681_r_at | ZFHX1B | 0.033 | 0.433 | 0.557 | 0.265 |
| 32587_at | ZFP36L2 | 0.137 | 0.088 | 0.099 | 0.023 |
| 32588_s_at | ZFP36L2 | 0.174 | 0.170 | 0.105 | 0.035 |
| 37254_at | ZNF133 | 0.195 | 0.016 | 0.162 | 0.029 |
| 35368_at | ZNF207 | 0.017 | 0.058 | 0.015 | 0.014 |
| 32034_at | ZNF217 | 0.160 | 0.374 | 0.091 | 0.024 |
| 31633_g_at | ZNF259 | 0.177 | 0.517 | 0.151 | 0.031 |

| A | B | G | H | I | J |
|---|---|---|---|---|---|
| Name | Gene Symbol | IM_IL13_2h_logFC | IM_IL13_6h_logFC | IM_IL13_12h_logFC | IM_IL13_24h_logFC |
| 1179_at | — | 0.330 | 0.185 | 0.744 | 0.153 |
| 32218_at | — | −0.712 | −0.579 | −0.422 | −0.420 |
| 32247_at | — | −0.082 | −0.393 | −0.109 | −0.867 |
| 1150_at | — | 1.264 | 1.696 | 1.916 | 1.765 |
| 1284_at | — | 1.281 | 1.269 | 1.186 | 1.383 |
| 40888_f_at | — | 0.674 | −0.160 | −0.413 | −0.737 |
| 953_g_at | — | 0.684 | 1.174 | 1.569 | 1.285 |
| 34145_at | — | −0.747 | −0.513 | −0.560 | −0.630 |
| 1173_g_at | — | −0.334 | −0.730 | −0.990 | −1.011 |
| 956_at | — | 0.848 | 0.971 | 0.704 | 0.800 |
| 1148_s_at | — | 0.473 | −0.445 | −1.876 | −2.336 |
| 38033_at | 38970 | −0.538 | −0.668 | −0.705 | −1.283 |
| 33173_g_at | 38971 | 0.409 | 0.917 | 0.940 | 1.006 |
| 160044_g_at | ACO2 | 0.164 | 0.469 | 0.577 | 1.047 |
| 40082_at | ACSL1 | 0.124 | 0.096 | −0.181 | −0.689 |
| 33881_at | ACSL3 | −0.132 | 0.394 | 0.752 | 0.337 |
| 39330_s_at | ACTN1 | 0.555 | 0.572 | 0.471 | 0.595 |
| 41654_at | ADA | −0.392 | −0.787 | −0.742 | −1.081 |
| 907_at | ADA | −0.337 | −0.645 | −0.628 | −1.078 |
| 35479_at | ADAM28 | −0.713 | −1.571 | −2.772 | −4.024 |
| 34378_at | ADFP | −0.809 | −0.455 | −0.420 | −0.891 |
| 34777_at | ADM | −0.482 | −0.452 | −0.943 | −1.135 |
| 40821_at | AHCY | 0.133 | 0.636 | 0.761 | 0.952 |
| 40516_at | AHR | 0.270 | 1.540 | 1.493 | 0.751 |
| 40789_at | AK2 | 0.944 | 0.879 | 1.370 | 1.021 |
| 38780_at | AKR1A1 | −0.025 | −0.266 | −0.646 | −0.608 |
| 36589_at | AKR1B1 | −0.150 | −0.276 | −0.637 | −0.897 |
| 37015_at | ALDH1A1 | −0.736 | −0.595 | −3.571 | −3.502 |
| 38315_at | ALDH1A2 | 1.394 | 1.824 | 0.957 | 0.910 |
| 40685_at | ALDH3B1 | −0.752 | −0.800 | −0.362 | −0.112 |
| 37330_at | ALDH4A1 | −0.047 | 0.502 | 0.374 | 0.605 |
| 34636_at | ALOX15 | 1.223 | 4.366 | 5.601 | 6.461 |
| 307_at | ALOX5 | −0.279 | −1.424 | −1.577 | −1.360 |
| 37099_at | ALOX5AP | −0.091 | −0.510 | −0.747 | −1.465 |
| 678_at | ALPPL2 | −0.021 | −0.312 | 0.235 | −0.600 |
| 38417_at | AMPD2 | 0.654 | 0.978 | 0.477 | 0.787 |
| 39315_at | ANGPT1 | 0.025 | −1.006 | −0.984 | −0.581 |
| 36637_at | ANXA11 | 0.006 | 0.484 | 0.501 | 0.677 |
| 37647_at | AOAH | −0.362 | −0.465 | −1.082 | −1.093 |
| 41549_s_at | AP1S2 | −0.609 | −0.865 | −0.448 | −0.737 |
| 37669_s_at | ATP1B1 | 0.335 | 2.133 | 1.424 | 1.500 |
| 37992_s_at | ATP5D | 0.299 | 0.059 | 0.094 | 0.593 |
| 34811_at | ATP5G3 | −0.218 | 0.372 | 0.713 | 0.455 |
| 38751_i_at | ATP5I | 0.177 | 0.203 | 0.155 | 0.651 |
| 36142_at | ATXN1 | −0.599 | 1.519 | 0.866 | 0.896 |
| 39942_at | BATF | 2.915 | 0.999 | 0.116 | 0.205 |
| 37971_at | BAZ1A | 0.238 | 0.485 | 0.600 | 0.615 |
| 36812_at | BCAR3 | −1.596 | 2.185 | 1.697 | 1.541 |
| 32828_at | BCKDK | 0.556 | 0.835 | 0.621 | 0.771 |
| 41356_at | BCL11A | 1.075 | 0.868 | 0.738 | 0.776 |
| 2002_s_at | BCL2A1 | 0.669 | −0.209 | −0.785 | −1.599 |
| 40091_at | BCL6 | 0.453 | 1.286 | 1.434 | 1.019 |
| 32842_at | BCL7A | 1.382 | 1.094 | 0.963 | 1.509 |
| 40879_at | BICD2 | −0.845 | −0.120 | 0.050 | 0.078 |
| 32726_g_at | BID | 0.976 | 0.823 | 0.706 | 0.732 |
| 32618_at | BLVRA | 0.762 | 0.976 | 0.908 | 1.011 |
| 41732_at | BOLA2 | 0.426 | 0.743 | 0.483 | 0.810 |
| 35615_at | BOP1 /// | 0.325 | 0.819 | 0.241 | 0.284 |
| | LOC653119 | | | | |
| 33759_at | BPGM | 0.058 | 0.180 | 0.911 | 0.208 |
| 41639_at | BRRN1 | 1.353 | 1.213 | 1.994 | 1.980 |
| 32675_at | BST1 | −0.330 | −1.067 | −1.453 | −1.577 |
| 38760_f_at | BTN3A2 | −0.654 | 0.139 | −0.131 | −0.612 |
| 41415_at | BYSL | 0.727 | 0.545 | 0.839 | 0.838 |
| 39172_at | C10orf22 | 0.354 | 0.723 | 0.939 | 0.841 |
| 38652_at | C10orf26 | −0.672 | −0.614 | −0.851 | −0.612 |
| 38411_at | C11orf32 | −0.498 | −1.713 | −1.593 | −1.499 |
| 41437_at | C14orf109 | −1.159 | −0.493 | −0.392 | −0.787 |
| 38969_at | C19orf10 | 0.464 | 0.764 | 0.191 | −0.075 |
| 41409_at | C1orf38 | −0.934 | −0.208 | 0.070 | −0.284 |
| 37668_at | C1QBP | 0.387 | 0.315 | 0.671 | 0.698 |
| 33374_at | C2 | 0.223 | 0.245 | −0.009 | 0.724 |
| 32107_at | C21orf25 | −0.581 | 0.645 | 0.885 | 0.825 |
| 31927_s_at | C21orf33 | −0.329 | 0.675 | 0.282 | 0.982 |
| 32068_at | C3AR1 | 0.220 | −1.402 | −1.709 | −1.494 |
| 40175_at | C3orf40 | 0.465 | 0.240 | 0.248 | 0.703 |
| 39710_at | C5orf13 | −0.020 | −1.169 | −0.644 | −0.326 |
| 41696_at | C7orf24 | 0.053 | 0.561 | 0.476 | 0.970 |
| 34995_at | CALCRL | 0.171 | 1.379 | 1.679 | 1.594 |
| 38716_at | CAMKK2 | −0.720 | −0.004 | −0.150 | −0.095 |
| 574_s_at | CASP1 | −1.229 | −1.238 | −1.299 | −1.614 |
| 39320_at | CASP1 | −1.445 | −1.823 | −1.316 | −1.662 |
| 37162_at | CCDC6 | −0.742 | 0.375 | 0.546 | 0.650 |
| 34183_at | CCDC69 | −0.463 | −0.636 | −0.199 | −0.044 |
| 37454_at | CCL13 | 1.382 | 2.236 | 2.754 | 5.200 |
| 1183_at | CCL17 | 3.438 | 4.691 | 5.206 | 4.757 |
| 32128_at | CCL18 | 2.988 | 4.707 | 4.269 | 5.530 |
| 875_g_at | CCL2 | 0.152 | −0.716 | −1.267 | −1.761 |
| 34375_at | CCL2 | 0.625 | −1.051 | −0.697 | −1.961 |
| 34041_at | CCL22 | 0.193 | 0.617 | 1.194 | 1.595 |
| 36445_at | CCL23 | 1.123 | 2.773 | 3.122 | 2.824 |
| 36444_s_at | CCL23 | 2.432 | 3.866 | 4.520 | 5.405 |
| 1924_at | CCNH | 1.847 | 0.870 | 0.959 | 0.954 |
| 39936_at | CCR2 /// | −1.127 | −0.186 | −0.589 | −0.730 |
| | LOC653518 | | | | |
| 35759_at | CCT2 | 0.424 | 0.639 | 0.579 | 0.376 |
| 39767_at | CCT8 | 0.140 | 0.231 | 0.277 | 0.601 |
| 36661_s_at | CD14 | −0.221 | −2.935 | −3.403 | −2.754 |
| 31438_s_at | CD163 | −0.386 | −2.323 | −2.986 | −2.200 |
| 34926_at | CD1A | 0.635 | 1.975 | 2.817 | 3.710 |
| 34927_at | CD1B | 0.772 | 4.155 | 5.859 | 6.060 |
| 37835_at | CD1C | 0.269 | 2.707 | 3.372 | 3.855 |
| 37861_at | CD1E | 0.890 | 4.459 | 5.731 | 5.786 |
| 34699_at | CD2AP | −0.681 | −1.058 | −0.900 | −0.745 |
| 34760_at | CD302 | −0.440 | −1.068 | −0.778 | −0.745 |
| 31870_at | CD37 | −0.212 | −0.595 | −0.834 | −1.156 |
| 31472_s_at | CD44 | 0.544 | 1.020 | 0.815 | 0.538 |
| 1125_s_at | CD44 | 0.820 | 1.062 | 0.338 | 0.435 |
| 1126_s_at | CD44 | 0.503 | 0.918 | 0.831 | 0.439 |
| 38006_at | CD48 | −1.441 | −0.912 | −1.063 | −1.034 |
| 39351_at | CD59 | 0.898 | −0.221 | −0.645 | −1.279 |
| 37536_at | CD83 | 2.381 | 1.482 | 1.313 | 1.428 |
| 505_at | CDC37 | 0.390 | 0.669 | 0.287 | 0.605 |
| 2031_s_at | CDKN1A | 2.272 | 1.871 | 2.001 | 1.565 |
| 36053_at | CDKN2C | 0.073 | −0.606 | −0.325 | −0.311 |
| 36190_at | CDR2 | 1.418 | 0.780 | 1.054 | 0.749 |
| 1052_s_at | CEBPD | −2.336 | −1.335 | −1.503 | −1.185 |
| 32589_at | CHAF1A | −0.231 | −0.664 | −0.622 | −0.397 |
| 33569_at | CLEC10A | 0.325 | 1.011 | 1.408 | 1.382 |
| 40698_at | CLEC2B | −0.986 | −0.982 | −0.898 | −0.768 |
| 40013_at | CLIC2 | 0.419 | 1.619 | 2.868 | 1.913 |
| 39960_at | COQ2 | −0.122 | −0.634 | −0.866 | −0.316 |
| 40427_at | COX17 | 0.664 | 0.400 | 0.544 | 0.648 |
| 39921_at | COX5B | 0.030 | 0.089 | 0.601 | 0.692 |
| 36687_at | COX7B | −0.045 | 0.301 | 0.696 | 0.606 |
| 39692_at | CREB3L2 | −0.375 | 0.105 | −0.948 | −0.862 |
| 39438_at | CREBL2 | 0.508 | 0.805 | 1.018 | 0.341 |
| 33232_at | CRIP1 | 0.083 | 0.331 | 1.313 | 0.817 |
| 40119_at | CRTAP | −0.290 | −0.473 | −0.683 | −0.692 |
| 34223_at | CSF3R | −0.186 | −0.719 | −1.284 | −1.105 |
| 596_s_at | CSF3R | −0.128 | −0.541 | −1.292 | −1.099 |
| 410_s_at | CSNK2B | 0.219 | 0.336 | 0.352 | 0.649 |
| 38112_g_at | CSPG2 | 0.155 | −1.752 | −2.854 | −2.573 |
| 31682_s_at | CSPG2 | −0.123 | −0.692 | −1.071 | −2.533 |
| 39581_at | CSTA | −0.308 | −0.868 | −1.306 | −1.347 |
| 35331_at | CTNNAL1 | 3.799 | 4.665 | 5.256 | 4.839 |
| 40444_s_at | CTNND1 | −0.863 | −0.750 | −0.632 | −0.376 |
| 36566_at | CTNS | 1.541 | 0.757 | 0.661 | 0.791 |
| 133_at | CTSC | 1.473 | 2.104 | 2.315 | 2.193 |
| 239_at | CTSD | −0.105 | −0.419 | −0.985 | −1.335 |
| 38466_at | CTSK | −0.533 | −1.392 | −2.422 | −3.439 |
| 41239_r_at | CTSS | −1.504 | −0.826 | −0.400 | −1.226 |
| 31823_at | CUTL1 | −0.192 | −0.528 | −0.111 | −0.606 |
| 40646_at | CX3CR1 | −1.518 | −2.578 | −1.904 | −2.433 |
| 37187_at | CXCL2 | 0.464 | −2.162 | −2.220 | −2.546 |
| 649_s_at | CXCR4 | −1.750 | −0.713 | −1.020 | −1.464 |
| 40296_at | CXorf9 | −0.234 | 0.604 | 0.609 | 0.863 |
| 999_at | CYP27A1 | −0.266 | −0.867 | −1.556 | −2.374 |
| 33389_at | CYP51A1 | 0.252 | 1.116 | 0.779 | 0.363 |
| 33753_at | DAAM1 | 0.962 | 0.337 | 0.844 | 1.187 |
| 1243_at | DDB2 | 0.513 | 0.584 | 0.607 | 0.697 |
| 38104_at | DECR1 | −0.199 | 0.179 | 0.583 | 0.612 |
| 41734_at | DENND3 | −0.529 | −0.726 | −1.029 | −0.816 |
| 41637_at | DEXI | −0.294 | −0.040 | −0.647 | −0.819 |
| 41872_at | DFNA5 | −1.073 | −1.812 | −1.573 | −2.516 |
| 39044_s_at | DGKD | −0.482 | −0.439 | −0.794 | −0.318 |
| 39814_s_at | DHRS7 | −0.376 | −0.860 | −0.644 | −0.753 |
| 41402_at | DKFZP564O0823 | 0.746 | 1.328 | 1.191 | 2.189 |
| 41716_at | DMXL2 | −1.261 | −0.649 | −1.201 | −1.078 |
| 35799_at | DNAJB9 | 0.967 | −0.480 | −0.328 | −0.141 |
| 40607_at | DPYSL2 | −0.289 | 0.623 | 0.612 | 0.608 |
| 32168_s_at | DSCR1 | 1.039 | 0.312 | 0.722 | 0.566 |
| 38555_at | DUSP10 | −3.037 | −1.626 | −1.805 | −1.845 |
| 41193_at | DUSP6 | −0.313 | −1.607 | −1.978 | −1.982 |
| 36921_at | DYNLT3 | −0.662 | −0.799 | −0.571 | −0.510 |
| 37016_at | ECHS1 | 0.017 | 0.259 | 0.481 | 0.667 |
| 40886_at | EEF1A1 /// | 0.332 | −0.337 | −0.135 | −0.704 |
| | APOLD1 /// | | | | |
| | LOC440595 | | | | |
| 37863_at | EGR2 | 1.209 | 2.427 | 3.073 | 2.380 |
| 33351_at | EIF1B | 0.797 | 0.138 | 0.740 | 0.348 |
| 34302_at | EIF3S4 | 0.395 | 0.400 | 0.350 | 0.706 |
| 35323_at | EIF3S9 | 0.478 | 0.341 | 0.127 | 0.585 |
| 37527_at | ELK3 | −0.868 | 0.074 | −0.345 | −0.349 |
| 40606_at | ELL2 | 1.381 | −0.560 | −0.246 | −0.845 |
| 39542_at | ENC1 | 0.246 | −0.491 | −0.773 | −0.686 |
| 32562_at | ENG | −0.712 | −0.356 | −0.815 | −0.601 |
| 41123_s_at | ENPP2 | 1.658 | 2.191 | 1.211 | 0.283 |
| 41124_r_at | ENPP2 | 1.207 | 1.512 | 1.054 | 0.167 |
| 32585_at | EPB41L2 | −0.147 | 1.867 | 1.538 | 1.311 |
| 902_at | EPHB2 | −0.867 | −1.232 | −1.126 | −0.813 |
| 41678_at | EPHB2 | −0.292 | −1.154 | −1.140 | −0.937 |
| 37731_at | EPS15 | −0.087 | 0.289 | 0.942 | 0.901 |
| 38158_at | ESPL1 | 2.106 | 0.930 | 1.099 | 1.391 |
| 38739_at | ETS2 | −0.185 | 0.141 | −0.658 | −1.145 |
| 32259_at | EZH1 | −0.309 | −0.174 | −0.797 | −0.291 |
| 40143_at | FAM53B | −1.655 | −0.635 | −0.292 | −0.050 |
| 32209_at | FAM89B | 0.066 | −0.018 | −0.005 | 0.711 |
| 38318_at | FAM8A1 | −1.381 | −0.323 | −0.615 | −0.617 |
| 36495_at | FBP1 | −0.445 | 0.130 | 0.346 | 0.961 |
| 34959_at | FCER2 | 2.017 | 4.040 | 5.794 | 5.810 |
| 34960_g_at | FCER2 | 2.075 | 3.613 | 5.248 | 5.484 |
| 37688_f_at | FCGR2A | 0.130 | −0.834 | −1.946 | −2.079 |
| 37689_s_at | FCGR2A | 0.556 | 0.010 | −2.088 | −2.374 |
| 37687_i_at | FCGR2B | −0.209 | −0.150 | −0.381 | −0.900 |
| 37200_at | FCGR3A | −0.683 | −1.259 | −2.101 | −1.990 |
| 31499_s_at | FCGR3B | −0.638 | −1.186 | −1.203 | −1.449 |
| 39593_at | FGL2 | 0.418 | 0.670 | 0.548 | 0.720 |
| 39591_s_at | FGL2 | 0.455 | 0.853 | 0.866 | 1.011 |
| 32546_at | FH | 0.025 | 0.475 | 0.650 | 0.697 |
| 880_at | FKBP1A | 0.660 | 1.194 | 1.235 | 1.289 |
| 41425_at | FLI1 | −1.806 | −1.337 | −1.009 | −0.697 |
| 41814_at | FUCA1 | −0.171 | −2.498 | −2.584 | −1.628 |
| 35338_at | FURIN | 0.381 | 0.362 | 0.826 | 1.135 |
| 34716_at | FUSIP1 /// | 0.660 | 0.177 | 0.218 | 0.228 |
| | LOC642558 | | | | |
| 41819_at | FYB | −1.508 | −1.435 | −0.997 | −1.086 |
| 38326_at | G0S2 | 3.184 | 5.128 | 3.090 | 2.013 |
| 33936_at | GALC | −0.603 | −0.732 | −0.567 | −0.570 |
| 1598_g_at | GAS6 | 0.806 | 1.663 | 1.794 | 1.750 |
| 1597_at | GAS6 | 2.378 | 2.936 | 2.484 | 2.239 |
| 37658_at | GAS6 | 0.545 | 1.637 | 1.846 | 1.881 |
| 33387_at | GAS7 | −1.674 | −1.296 | −1.102 | −1.086 |
| 36596_r_at | GATM | −0.647 | −0.946 | −0.958 | −1.264 |
| 32643_at | GBE1 | −0.378 | 0.006 | −0.143 | −0.633 |
| 32700_at | GBP2 | −0.007 | −0.763 | −0.687 | −0.528 |
| 37944_at | GCH1 | 0.323 | 0.307 | −1.337 | −1.692 |
| 38237_at | GGTLA1 | 0.278 | 0.975 | 1.516 | 1.063 |
| 34311_at | GLRX | −2.011 | −0.700 | −0.519 | −0.724 |
| 40522_at | GLUL | −1.806 | −1.220 | −1.022 | −0.800 |
| 31812_at | GMPR | 0.202 | −0.800 | −0.637 | −0.166 |
| 35272_at | GNG5 | −0.176 | 0.050 | 0.640 | 0.731 |
| 38379_at | GPNMB | −0.811 | −1.269 | −2.347 | −2.005 |
| 31700_at | GPR35 | 0.591 | 0.993 | 0.674 | 0.537 |
| 34930_at | GPR65 | −1.828 | −1.348 | −0.609 | −0.932 |
| 40994_at | GRK5 | −0.077 | −0.501 | −0.833 | −0.857 |
| 33932_at | GSPT1 | 0.399 | 0.393 | 0.673 | 0.515 |
| 869_at | GTF2A2 | 0.244 | 0.392 | 0.786 | 0.949 |
| 35821_at | HDAC3 | −0.501 | 0.598 | 0.669 | 0.408 |
| 40121_at | HIP2 | −1.165 | 0.160 | 0.192 | 0.103 |
| 32980_f_at | HIST1H2BC | −0.032 | −0.121 | −0.548 | −0.744 |
| 31522_f_at | HIST1H2BF | −0.419 | −0.034 | −0.420 | −0.988 |
| 31524_f_at | HIST1H2BI | −0.197 | −0.376 | −0.660 | −1.013 |
| 35576_f_at | HIST1H2BL | −0.097 | −0.280 | −0.637 | −1.082 |
| 31528_f_at | HIST1H2BM | −0.051 | 0.450 | −0.628 | −0.879 |
| 36347_f_at | HIST1H2BN | 0.007 | −0.101 | −0.732 | −0.987 |
| 40964_at | HK2 | −2.036 | −1.109 | −1.243 | −1.587 |
| 37604_at | HNMT | −0.891 | −0.392 | −0.427 | −0.762 |
| 38292_at | HOMER2 | 0.844 | 0.747 | 2.044 | 2.571 |
| 38233_at | HOMER3 | −1.160 | −1.518 | −1.266 | −0.974 |
| 36030_at | HOM-TES- | −0.901 | −0.634 | −0.618 | −0.458 |
| | 103 | | | | |
| 35702_at | HSD11B1 | 0.581 | 2.941 | 3.395 | 2.768 |
| 32316_s_at | HSP90AA1 | 0.648 | 0.517 | 0.768 | 0.321 |
| 1161_at | HSP90AB1 | 0.706 | 0.822 | 0.824 | 0.233 |
| 33984_at | HSP90AB1 | 0.554 | 0.465 | 0.619 | 0.439 |
| 31692_at | HSPA1A | 0.357 | 0.251 | 0.774 | 0.250 |
| 35965_at | HSPA6 | −0.397 | −1.058 | −1.622 | −1.293 |
| 117_at | HSPA6 /// | −0.220 | −0.883 | −1.751 | −1.240 |
| | LOC652878 | | | | |
| 40637_at | HSPA8 | −0.129 | 0.134 | 1.142 | 0.220 |
| 36785_at | HSPB1 | 0.396 | 1.049 | 1.726 | 1.174 |
| 41259_at | HSPC111 | 0.991 | 0.825 | 1.063 | 0.871 |
| 37720_at | HSPD1 | 0.508 | 0.517 | 0.622 | 0.617 |
| 39353_at | HSPE1 | 0.393 | 0.355 | 0.921 | 0.447 |
| 719_g_at | HTRA1 | −1.087 | −3.221 | −4.095 | −3.351 |
| 718_at | HTRA1 | −1.094 | −2.684 | −3.037 | −3.426 |
| 36564_at | IBRDC3 | 1.477 | 1.178 | 0.954 | 0.948 |
| 38454_g_at | ICAM2 | −0.146 | −0.134 | −0.494 | −0.962 |
| 37043_at | ID3 | −0.873 | −0.666 | −1.026 | −1.390 |
| 36927_at | IFI44L | −0.781 | −1.288 | −1.819 | −2.243 |
| 676_g_at | IFITM1 /// | 0.152 | −0.390 | −1.142 | −1.154 |
| | IFITM3 /// | | | | |
| | IFITM2 | | | | |
| 41745_at | IFITM3 | 0.583 | −0.608 | −1.757 | −1.589 |
| 1038_s_at | IFNGR1 | −2.252 | −0.974 | −0.624 | −0.567 |
| 34946_at | IGSF6 | 0.319 | 0.411 | 0.857 | 0.516 |
| 1061_at | IL10RA | 0.662 | 0.511 | 0.289 | 0.484 |
| 359_at | IL13RA1 | −0.496 | −0.487 | −0.513 | −0.732 |
| 1165_at | IL18 | −0.047 | −0.889 | −0.916 | −1.360 |
| 39402_at | IL1B | 1.842 | 0.216 | −0.987 | −1.022 |
| 1368_at | IL1R1 | 1.259 | 1.706 | 2.516 | 2.172 |
| 998_s_at | IL1R2 | 0.270 | 1.962 | 2.469 | 1.816 |
| 37603_at | IL1RN | 3.108 | 1.991 | 1.202 | 1.079 |
| 37844_at | IL27RA | −0.349 | 0.888 | 1.175 | 1.227 |
| 37843_i_at | IL27RA | 0.084 | 0.812 | 0.911 | 0.916 |
| 1185_at | IL3RA | 1.177 | 1.749 | 1.165 | 0.881 |
| 1369_s_at | IL8 | 0.342 | −0.721 | −1.721 | −3.129 |
| 37276_at | IQGAP2 | −0.747 | −0.598 | −0.351 | −0.670 |
| 37625_at | IRF4 | 3.332 | 0.572 | 1.194 | 0.463 |
| 35731_at | ITGA4 | 0.506 | −1.409 | −1.367 | −1.446 |
| 38533_s_at | ITGAM | 1.434 | 2.776 | 2.002 | 1.657 |
| 36709_at | ITGAX | 0.437 | 0.760 | 0.600 | 1.157 |
| 41300_s_at | ITM2B | 0.003 | −0.787 | −0.913 | −0.902 |
| 32778_at | ITPR1 | −1.051 | −1.054 | −1.337 | −0.909 |
| 755_at | ITPR1 | −0.380 | −1.181 | −1.087 | −1.104 |
| 33178_at | JAG1 | 0.688 | 0.116 | 0.167 | 0.169 |
| 35414_s_at | JAG1 | 2.928 | 1.783 | 1.140 | 2.075 |
| 34786_at | JMJD1A | −1.401 | −0.250 | −0.367 | −0.158 |
| 38972_at | KCTD12 | −1.743 | −1.441 | −1.245 | −1.341 |
| 39783_at | KIAA0100 | 0.668 | 0.684 | 1.094 | 1.494 |
| 35744_at | KIAA0141 | −0.145 | −0.037 | −0.622 | 0.058 |
| 31863_at | KIAA0179 | −0.463 | 0.451 | 1.184 | 2.030 |
| 38735_at | KIAA0513 | −1.295 | −0.286 | −0.376 | −0.199 |
| 35252_at | KIAA0528 | −0.072 | −0.241 | −0.189 | −0.680 |
| 39559_at | KMO | 0.428 | 1.082 | 1.222 | 0.801 |
| 180_at | LENG4 | 0.108 | 0.565 | 0.685 | 1.357 |
| 37542_at | LHFPL2 | −0.992 | −1.213 | −1.493 | −2.161 |
| 38618_at | LIMK2 /// | 0.450 | 0.422 | 0.627 | 0.861 |
| | PPP1R14BP1 | | | | |
| 39232_at | LIMS1 | −0.020 | 0.665 | 1.006 | 0.680 |
| 38745_at | LIPA | 0.073 | 1.446 | 1.409 | 0.580 |
| 31936_s_at | LKAP | −1.714 | −0.392 | −0.152 | 0.161 |
| 32195_at | LOC339287 | −0.274 | −0.608 | −0.357 | 0.041 |
| 39879_s_at | LOC388397 | 0.121 | −0.148 | −0.383 | −0.702 |
| 33866_at | LOC643634 | 0.196 | 0.684 | 1.153 | 1.198 |
| 39937_at | LOC653518 | −0.919 | 0.077 | −0.425 | −0.736 |
| 38775_at | LRP1 | −0.058 | −0.514 | −1.064 | −0.711 |
| 41320_s_at | LRRFIP1 | 0.055 | 0.512 | 0.983 | 0.543 |
| 36493_at | LSP1 /// | −0.054 | −0.190 | 0.564 | 0.806 |
| | LOC649377 | | | | |
| 38081_at | LTA4H | −0.290 | −1.319 | −1.518 | −1.695 |
| 35869_at | LY86 | −0.118 | −0.692 | −1.106 | −1.082 |
| 41505_r_at | MAF | 2.097 | 1.219 | 1.287 | 0.336 |
| 36711_at | MAFF | 3.314 | 3.010 | 2.655 | 1.982 |
| 37472_at | MANBA | −0.779 | −0.947 | −0.742 | −0.361 |
| 41772_at | MAOA | 5.016 | 5.167 | 5.876 | 4.585 |
| 41771_g_at | MAOA | 4.387 | 4.419 | 3.905 | 4.392 |
| 41770_at | MAOA | 6.053 | 6.013 | 5.609 | 5.845 |
| 976_s_at | MAPK1 | 0.764 | 0.277 | 0.778 | 0.577 |
| 33223_at | MAST3 | −0.710 | −0.289 | −0.657 | 0.072 |
| 32571_at | MAT2A | 1.603 | 1.004 | 1.380 | 1.253 |
| 34386_at | MBD4 | −1.120 | −0.181 | −0.031 | −0.198 |
| 36608_at | MDH1 | 0.071 | 0.802 | 1.013 | 0.984 |
| 35629_at | MKL1 | −0.986 | 0.848 | 0.455 | 0.788 |
| 32207_at | MPP1 | −0.472 | −1.077 | −1.135 | −0.734 |
| 36908_at | MRC1 /// | 3.727 | 3.882 | 3.310 | 2.256 |
| | MRC1L1 | | | | |
| 39812_at | MRPL12 | −0.040 | 0.785 | 0.643 | 0.613 |
| 35992_at | MSC | 1.931 | 1.481 | 1.816 | 1.650 |
| 674_g_at | MTHFD1 | −0.978 | 0.260 | 0.618 | 0.683 |
| 40074_at | MTHFD2 | 0.069 | 0.562 | 0.912 | 0.875 |
| 879_at | MX2 | −0.274 | −1.122 | −1.168 | −0.860 |
| 1973_s_at | MYC | 0.964 | 0.527 | 0.917 | 0.544 |
| 38369_at | MYD88 | −1.354 | −0.750 | −0.515 | 0.034 |
| 32069_at | N4BP1 | 1.119 | 0.558 | 0.894 | 0.446 |
| 41249_at | NADK | −1.294 | −0.397 | −0.360 | −0.384 |
| 36607_at | NAGA | −0.247 | 0.276 | 0.528 | 0.648 |
| 38187_at | NAT1 | −0.095 | 0.380 | 0.641 | 0.142 |
| 34279_at | NBPF14 /// | −0.289 | 0.071 | −0.563 | −1.073 |
| | NBPF1 /// | | | | |
| | KIAA1245 /// | | | | |
| | NBPF11 /// | | | | |
| | NBPF15 /// | | | | |
| | NBPF20 /// | | | | |
| | NBPF9 /// | | | | |
| | NBPF10 /// | | | | |
| | NBPF12 /// | | | | |
| | NBPF8 /// | | | | |
| | NBPF16 | | | | |
| 39174_at | NCOA4 | −0.471 | −0.737 | −0.718 | −0.730 |
| 39358_at | NCOR2 | 0.075 | 0.913 | 0.376 | 0.612 |
| 38257_at | NDUFS8 | −0.271 | 0.299 | 0.407 | 0.700 |
| 34893_at | NDUFV2 | 1.058 | 0.452 | 0.476 | 0.451 |
| 37544_at | NFIL3 | 0.367 | 0.970 | 1.346 | 1.239 |
| 35366_at | NID1 | −0.603 | −1.614 | −1.851 | −2.727 |
| 39073_at | NME1 | 0.486 | 1.001 | 0.770 | 0.675 |
| 1985_s_at | NME1 | 0.658 | 1.225 | 1.008 | 0.756 |
| 1979_s_at | NOL1 | −0.021 | 0.881 | 0.721 | 0.595 |
| 32719_at | NRG1 | 0.355 | −0.496 | −1.730 | −1.935 |
| 40088_at | NRIP1 | −0.636 | −0.778 | −0.831 | −0.777 |
| 32644_at | NUP188 | 0.468 | 0.731 | 0.328 | 0.738 |
| 40768_s_at | NUP214 | −1.118 | −0.496 | −1.350 | −1.040 |
| 34491_at | OASL | 0.551 | −0.374 | −0.603 | −1.103 |
| 36134_at | OLFM1 | −1.492 | −0.599 | −1.406 | −2.322 |
| 38855_s_at | OLFM1 | −1.145 | −0.574 | −1.702 | −2.963 |
| 36007_at | OLFML2B | −1.198 | −2.206 | −1.933 | −2.125 |
| 36689_at | OSBPL1A | −1.147 | −0.319 | −0.929 | −0.798 |
| 35674_at | PADI2 | −0.076 | −1.383 | −1.941 | −2.041 |
| 39056_at | PAICS | −0.114 | 0.936 | 0.424 | 0.605 |
| 1560_g_at | PAK2 | −0.212 | 0.691 | 0.694 | 0.505 |
| 41191_at | PALLD | 2.563 | 2.039 | 2.498 | 2.836 |
| 38465_at | PAM | −0.521 | −0.802 | −1.060 | −1.140 |
| 34352_at | PCBD1 | 0.514 | 0.597 | 0.714 | 0.742 |
| 37188_at | PCK2 | −0.893 | −0.634 | −0.838 | −0.358 |
| 1884_s_at | PCNA | −1.090 | −0.049 | 0.143 | −0.020 |
| 32212_at | PDCD8 | 0.064 | 0.601 | 0.634 | 0.661 |
| 746_at | PDE3B | 0.164 | −0.365 | −1.685 | −1.038 |
| 36092_at | PDE4DIP | −0.937 | −0.439 | −0.493 | −1.962 |
| 35714_at | PDXK | 0.485 | 1.070 | 0.705 | 0.890 |
| 37397_at | PECAM1 | −0.384 | −1.361 | −1.216 | −0.751 |
| 32455_s_at | PELP1 | 0.646 | 1.287 | 0.883 | 0.900 |
| 39175_at | PFKP | 1.512 | 1.775 | 2.521 | 2.614 |
| 36502_at | PFTK1 | −0.419 | −0.130 | −0.874 | −1.257 |
| 32739_at | PGM3 | 0.222 | −0.084 | −0.596 | −0.273 |
| 33333_at | PIP3-E | 1.079 | 0.799 | 1.004 | 1.285 |
| 34839_at | PITRM1 | 0.969 | 1.013 | 0.998 | 0.899 |
| 33707_at | PLA2G4C | −0.255 | −0.315 | −0.272 | −0.738 |
| 36943_r_at | PLAGL1 | −0.634 | −0.114 | −0.091 | −0.604 |
| 37310_at | PLAU | 1.541 | 0.504 | 0.406 | −0.165 |
| 32775_r_at | PLSCR1 | 0.004 | −0.697 | −0.692 | −1.138 |
| 32193_at | PLXNC1 | −0.990 | −1.740 | −1.623 | −1.609 |
| 38653_at | PMP22 | −0.273 | −1.545 | −1.739 | −1.991 |
| 1696_at | POLB | −0.697 | −0.695 | −0.797 | −0.734 |
| 858_at | POR | 0.293 | 0.972 | 0.184 | 0.244 |
| 37104_at | PPARG | 3.752 | 1.668 | 1.585 | 1.412 |
| 41709_at | PPFIBP2 | 0.733 | 0.675 | 0.391 | 0.507 |
| 37384_at | PPM1F | 0.198 | 0.281 | 0.699 | 0.746 |
| 33358_at | PPM1H | −0.391 | −0.804 | −0.803 | −0.784 |
| 41540_at | PPP1R7 | 0.098 | 0.473 | 0.698 | 0.949 |
| 1336_s_at | PRKCB1 | −0.491 | −0.680 | −0.836 | −1.051 |
| 1217_g_at | PRKCB1 | −0.308 | −0.789 | −0.337 | −0.728 |
| 160029_at | PRKCB1 | −0.314 | −0.720 | −0.647 | −0.750 |
| 37969_at | PTGS1 | 0.023 | 0.434 | 0.454 | 0.781 |
| 33804_at | PTK2B | −0.387 | −0.774 | −0.450 | −0.742 |
| 35342_at | PTPLB | −0.320 | 0.275 | 0.685 | 1.040 |
| 36808_at | PTPN22 | −1.113 | −2.064 | −1.394 | −1.738 |
| 39672_at | PTPN7 | 0.245 | 0.451 | 0.669 | 0.851 |
| 40519_at | PTPRC | −0.634 | −0.286 | −0.613 | −0.675 |
| 32916_at | PTPRE | 1.553 | 2.044 | 2.201 | 2.131 |
| 32199_at | PTPRO | 0.559 | 0.610 | 0.922 | 1.587 |
| 1190_at | PTPRO | −0.638 | 0.859 | 1.200 | 1.499 |
| 35966_at | QPCT | 1.223 | 2.041 | 2.487 | 2.040 |
| 37978_at | QPRT | −0.155 | 0.817 | 1.427 | 2.492 |
| 1257_s_at | QSCN6 | 1.453 | 2.202 | 1.285 | 1.518 |
| 809_at | RAB27A | −0.448 | −0.603 | −0.801 | −0.704 |
| 1202_g_at | RAB33A | 1.154 | 1.664 | 0.912 | 1.148 |
| 35340_at | RAB8A | 0.208 | 0.883 | 1.027 | 0.685 |
| 35339_at | RAB8A | 0.362 | 0.918 | 0.974 | 0.707 |
| 35289_at | RABGAP1 | −0.561 | 0.344 | 0.560 | 0.674 |
| 34445_at | RABGAP1L | −0.716 | −0.659 | −1.052 | −0.980 |
| 37703_at | RABGGTB | −0.205 | 0.378 | 0.442 | 0.618 |
| 1874_at | RAD23B | 0.037 | 0.374 | 0.215 | 0.615 |
| 32593_at | RAFTLIN | −0.217 | 0.996 | 1.060 | 0.871 |
| 35668_at | RAMP1 | 1.545 | 1.682 | 2.837 | 5.185 |
| 41342_at | RANBP1 | −0.418 | 0.152 | 0.828 | −0.103 |
| 34745_at | RAPGEF2 | 0.999 | 0.898 | 0.446 | 0.522 |
| 32026_s_at | RAPGEF2 | 1.480 | 1.204 | 0.766 | 0.719 |
| 1675_at | RASA1 | −1.593 | −0.340 | −0.317 | 0.206 |
| 36935_at | RASA1 | −1.094 | −0.392 | −0.287 | 0.123 |
| 37598_at | RASSF2 | −0.798 | −0.133 | −0.012 | −0.098 |
| 34187_at | RBMS2 | 0.565 | 0.476 | 0.403 | 2.136 |
| 40818_at | RBPSUH | 0.250 | 0.858 | 0.506 | 0.805 |
| 35193_at | RCBTB2 | −1.303 | −0.920 | −1.025 | −1.009 |
| 41172_at | RDH11 | −1.447 | −0.606 | −0.626 | −0.124 |
| 38908_s_at | REV3L | −0.933 | −0.752 | −0.758 | −0.606 |
| 37701_at | RGS2 | −2.969 | −2.343 | −2.058 | −1.352 |
| 36550_at | RIN2 | −1.278 | −1.373 | −0.832 | −0.643 |
| 32664_at | RNASE4 | −0.590 | −2.375 | −2.044 | −2.244 |
| 35777_at | RNF4 | 0.157 | 0.540 | 0.318 | 0.674 |
| 36187_at | RNH1 | 0.388 | 0.592 | 0.742 | 0.824 |
| 41296_s_at | RPS24 | −0.064 | 0.639 | 0.799 | 0.622 |
| 33325_at | RPS6KA2 | −0.491 | −0.905 | −0.942 | −0.328 |
| 32544_s_at | RSU1 | −0.291 | −0.075 | 0.429 | 0.783 |
| 106_at | RUNX3 | −0.361 | −0.493 | −0.628 | −0.726 |
| 37732_at | RYBP | 0.564 | 0.466 | 0.660 | 0.642 |
| 539_at | RYK | −0.299 | 0.759 | 0.646 | 0.533 |
| 41096_at | S100A8 | 0.262 | −0.762 | −2.508 | −2.514 |
| 41471_at | S100A9 | 0.083 | −0.336 | −1.899 | −3.154 |
| 34304_s_at | SAT | −0.986 | −1.114 | −0.879 | −1.113 |
| 41200_at | SCARB1 | 1.063 | 1.487 | 1.636 | 1.368 |
| 36192_at | SCRN1 | −0.282 | 0.852 | 0.937 | 0.403 |
| 39757_at | SDC2 | −0.239 | 0.891 | 1.206 | 1.502 |
| 40390_at | SDS | −1.714 | −0.912 | −1.223 | −1.648 |
| 41597_s_at | SEC22B | −0.417 | −0.319 | −0.490 | −0.942 |
| 245_at | SELL | −0.084 | −0.354 | −1.001 | −1.395 |
| 34363_at | SEPP1 | −0.766 | −2.841 | −3.734 | −4.896 |
| 37185_at | SERPINB2 | 0.268 | −1.032 | −2.042 | −1.963 |
| 34438_at | SERPINB9 | 1.085 | 0.663 | 0.736 | −0.057 |
| 40856_at | SERPINF1 | −0.521 | −0.869 | −0.827 | −1.279 |
| 40638_at | SFPQ | 0.675 | 0.462 | 0.657 | 0.293 |
| 40457_at | SFRS3 | 0.078 | 0.137 | 0.614 | 0.264 |
| 973_at | SGK | −1.668 | −1.612 | −1.352 | −1.078 |
| 38968_at | SH3BP5 | −0.997 | −0.808 | −1.566 | −1.216 |
| 1427_g_at | SLA | 1.632 | 1.300 | 1.236 | 1.090 |
| 1426_at | SLA | 1.799 | 1.193 | 1.328 | 1.302 |
| 1138_at | SLC20A1 | 1.430 | 1.452 | 1.551 | 0.825 |
| 38122_at | SLC23A2 | −1.005 | −0.357 | −0.826 | −0.937 |
| 37740_r_at | SLC25A5 | −0.784 | 0.275 | 0.775 | 0.149 |
| 36979_at | SLC2A3 | 2.286 | 0.375 | 0.406 | 0.380 |
| 34749_at | SLC31A2 | −1.717 | −0.524 | −0.222 | −0.155 |
| 37895_at | SLC35A1 | −1.144 | −0.946 | −0.765 | −0.718 |
| 1798_at | SLC39A6 | −0.246 | 0.345 | 0.982 | 0.608 |
| 33731_at | SLC7A7 | −0.784 | −0.968 | −1.761 | −1.584 |
| 40810_at | SMARCC1 | −0.283 | 0.146 | 0.360 | 0.695 |
| 39950_at | SMPDL3A | 2.182 | −0.314 | −0.716 | −1.024 |
| 33354_at | SMURF2 | −0.512 | −0.270 | −0.023 | −0.743 |
| 40842_at | SNRPA | 0.064 | 0.411 | 0.265 | 0.588 |
| 40605_at | SNX4 | −0.420 | 0.066 | 0.650 | 0.148 |
| 41592_at | SOCS1 | 5.292 | 4.291 | 4.244 | 3.686 |
| 32140_at | SORL1 | −0.385 | −1.360 | −1.349 | −1.727 |
| 41573_at | SP3 | −1.098 | −0.538 | −0.413 | −0.925 |
| 671_at | SPARC | 0.028 | −0.292 | −2.186 | −2.901 |
| 1685_at | SPHAR | −0.252 | −0.707 | −0.097 | 0.148 |
| 33448_at | SPINT1 | 0.121 | 0.651 | 0.718 | 0.946 |
| 34348_at | SPINT2 | 1.117 | 1.881 | 2.025 | 2.044 |
| 36798_g_at | SPN | 0.833 | 0.996 | 0.898 | 0.767 |
| 34342_s_at | SPP1 | −0.112 | −0.677 | −2.198 | −3.127 |
| 2092_s_at | SPP1 | −0.022 | −0.406 | −2.486 | −3.069 |
| 32135_at | SREBF1 | 0.675 | 0.353 | 0.465 | 0.621 |
| 40109_at | SRF | 0.692 | 0.171 | 0.466 | 0.669 |
| 35231_at | SRP19 | 0.639 | 0.186 | 0.315 | 0.281 |
| 1640_at | ST13 | −0.188 | 0.335 | 0.668 | 0.546 |
| 39298_at | ST3GAL6 | −2.618 | −1.441 | −1.096 | −0.845 |
| 38487_at | STAB1 | −0.205 | −0.665 | −1.491 | −0.766 |
| 38525_at | STAM2 | −0.848 | 1.372 | 1.285 | 0.313 |
| 41295_at | STARD7 | −0.312 | 0.784 | 0.811 | 0.583 |
| AFFX- | STAT1 | −0.577 | −0.753 | −0.660 | −0.956 |
| HUMISGF3A/ | | | | | |
| M | | | | | |
| 97935_3_at | | | | | |
| 33339_g_at | STAT1 | −0.242 | −0.996 | −1.220 | −1.152 |
| 39708_at | STAT3 | −0.205 | −0.043 | −0.041 | −0.770 |
| 40473_at | STK24 | −1.199 | −0.329 | −0.222 | −0.192 |
| 32182_at | STK38L | −0.740 | −0.707 | −0.581 | −0.226 |
| 41663_at | STX6 | −1.654 | −0.747 | −0.701 | −0.348 |
| 41034_s_at | SULT2B1 | −0.080 | −0.220 | −0.171 | −0.596 |
| 31869_at | SWAP70 | −1.360 | −0.526 | −0.438 | −0.725 |
| 34885_at | SYNGR2 | 0.773 | 0.883 | 0.618 | 0.606 |
| 36532_at | SYNJ2 | 1.023 | 0.640 | 1.040 | 0.523 |
| 34966_at | T | 0.153 | −0.252 | −1.194 | −0.509 |
| 39416_at | TAX1BP3 | 0.039 | 0.271 | 0.584 | 0.637 |
| 38317_at | TCEAL1 | −0.101 | 0.035 | −0.313 | −0.702 |
| 40865_at | TDG | 0.208 | 0.177 | 0.264 | 0.712 |
| 160025_at | TGFA | 2.013 | 0.962 | 0.995 | 0.635 |
| 38805_at | TGIF | −0.960 | −0.634 | −0.561 | −0.235 |
| 38404_at | TGM2 | 3.912 | 4.662 | 2.549 | 2.374 |
| 32829_at | TIMM17A | 0.658 | 0.713 | 0.729 | 0.385 |
| 39411_at | TIPARP | −0.145 | −0.396 | −0.434 | −0.854 |
| 38364_at | TLE4 | −1.732 | −0.552 | −0.188 | −0.341 |
| 40310_at | TLR2 | −0.325 | −0.716 | −0.508 | −0.745 |
| 34473_at | TLR5 | −1.934 | −1.040 | −1.113 | −0.927 |
| 32116_at | TMC6 | 0.288 | 1.052 | 0.625 | 0.471 |
| 36950_at | TMED9 | 0.431 | 0.618 | 0.104 | 0.327 |
| 39424_at | TNFRSF14 | 0.447 | 0.587 | 0.392 | 0.392 |
| 1583_at | TNFRSF1B | −1.060 | −0.657 | −0.610 | −0.366 |
| 33813_at | TNFRSF1B | −0.878 | −0.642 | −0.555 | −0.332 |
| 1715_at | TNFSF10 | −1.391 | −1.609 | −1.527 | −1.569 |
| 1030_s_at | TOP1 | 0.809 | 0.256 | 0.521 | 0.392 |
| 31680_at | TOP1P2 | 0.806 | 0.209 | 0.372 | 0.471 |
| 36139_at | TRAF3IP2 | 0.000 | −0.620 | −0.136 | −0.721 |
| 35238_at | TRAF5 | 0.116 | −0.342 | 0.104 | 0.653 |
| 1468_at | TRAP1 | 0.206 | 0.684 | 0.451 | 0.794 |
| 41468_at | TRGC2 /// | 3.157 | 1.155 | −0.259 | −0.887 |
| | TRGV2 /// | | | | |
| | TRGV9 /// | | | | |
| | TARP /// | | | | |
| | LOC642083 | | | | |
| 36825_at | TRIM22 | −0.288 | −0.815 | −1.300 | −1.510 |
| 39032_at | TSC22D1 | −1.244 | −0.292 | −0.532 | −0.912 |
| 36629_at | TSC22D3 | −0.809 | −0.930 | −0.761 | −0.765 |
| 32730_at | TSPYL5 | 0.663 | 1.029 | 1.351 | 0.817 |
| 34825_at | TTRAP | −0.976 | −0.223 | −0.247 | −0.635 |
| 38350_f_at | TUBA2 | 0.300 | 0.459 | 0.950 | 0.989 |
| 40567_at | TUBA3 | 0.762 | 0.661 | 1.324 | 1.412 |
| 151_s_at | TUBB | 0.264 | 0.133 | 0.491 | 0.758 |
| 429_f_at | TUBB2A /// | 0.489 | 0.739 | 0.920 | 0.842 |
| | TUBB4 /// | | | | |
| | TUBB2B | | | | |
| 33678_i_at | TUBB2C | 0.856 | 1.079 | 1.235 | 0.980 |
| 33679_f_at | TUBB2C | 0.520 | 0.706 | 0.764 | 0.695 |
| 471_f_at | TUBB3 | 0.482 | 0.367 | 0.798 | 0.865 |
| 38089_at | UBAP2L | 0.395 | 0.557 | 0.782 | 0.898 |
| 39040_at | UBE2J1 | 0.838 | 0.887 | 0.602 | 0.486 |
| 223_at | UBE2L3 | 0.135 | 0.488 | 0.672 | 0.549 |
| 40505_at | UBE2L6 | −0.334 | −0.233 | −0.587 | −0.649 |
| 40839_at | UBL3 | 1.038 | 0.127 | 0.414 | 0.963 |
| 39442_at | UNC50 | −0.891 | −0.220 | 0.102 | 0.001 |
| 283_at | UQCRC1 | 0.250 | 0.404 | 0.636 | 0.664 |
| 41859_at | UST | 0.257 | 1.530 | 0.919 | 0.566 |
| 34481_at | VAV1 | −1.259 | −0.253 | −0.590 | −0.140 |
| 36601_at | VCL | 0.382 | 0.483 | 0.772 | 1.002 |
| 31608_g_at | VDAC1 | 0.214 | 0.514 | 0.616 | 0.760 |
| 40198_at | VDAC1 | 0.259 | 0.581 | 0.724 | 0.571 |
| 1388_g_at | VDR | 1.280 | 1.114 | 1.426 | 1.274 |
| 1410_at | VDR | 0.985 | 1.802 | 2.171 | 2.136 |
| 34498_at | VNN2 | −0.995 | −2.501 | −2.237 | −2.248 |
| 1669_at | WNT5A | 1.334 | 2.827 | 3.306 | 4.272 |
| 31862_at | WNT5A | 2.419 | 2.916 | 3.665 | 3.705 |
| 40167_s_at | WSB2 | 0.034 | 0.437 | 0.796 | 0.392 |
| 783_at | WWP1 | −0.966 | −1.303 | −1.261 | −1.309 |
| 784_g_at | WWP1 | −0.806 | −1.226 | −1.179 | −1.071 |
| 39755_at | XBP1 | 1.363 | 0.607 | 0.135 | −0.122 |
| 39756_g_at | XBP1 | 1.338 | 0.591 | 0.222 | −0.218 |
| 41669_at | ZCCHC11 | −0.681 | −0.289 | −0.610 | −0.731 |
| 35681_r_at | ZFHX1B | −1.993 | −0.430 | −0.044 | −0.200 |
| 32587_at | ZFP36L2 | −1.682 | −1.343 | −1.209 | −1.351 |
| 32588_s_at | ZFP36L2 | −1.116 | −1.188 | −1.405 | −1.414 |
| 37254_at | ZNF133 | −0.849 | −0.641 | −0.347 | −0.488 |
| 35368_at | ZNF207 | 0.630 | 0.367 | 0.620 | 0.356 |
| 32034_at | ZNF217 | −1.595 | 0.351 | 0.622 | 0.811 |
| 31633_g_at | ZNF259 | 0.697 | 0.095 | 0.359 | 0.707 |

TABLE 8

22209_at : target sequence is located in intron4 of C6ORF62 gene
Intron4 fasta sequence:
GTATTTTGGTCTAAAGTGTGATGAGTATTTCAATATGTGAAAACTACTAGAATATAATAG
GGTCTAACTTGAGAAATTCTTTGGGAAAATGGTTTCTGATAGTTTTATTTCACGAGTCTC
CCCTATTTAGAATATTGTGATGCAAGAGAAGAAAGCGTTTGGATTATAGAATCTCTTGAC
AGTGTGGTGGTTCCACCTGCCCAGTGTGGCTTTGAAATTATGACTAGAGAAAATCTTTTA
AAGTGGACATTTACTGATTTATAGAGGGGCCCACAGATGAGCTTCTGAGATCTGTAACTC
TTGAAGCCTTCACCACACATCCTTCTAAAACCGTATATTTAACTGCTGCTTCCCAAAGGA
ATGTGATCTGAAATGGGTGAAGAAATCATTTTGTAGAAGTTGATCTGTATATAAAATTAT
AGAAGAAAGAAGTAAATTTAGTAGTCATTCTTAACCTTAAAATCTTGCTGACTTTTGACT
GTTTGTCATGGTATACTAGACATTGCTCAAGTGAATCCCCCCTCTAGTGTTAAGGGCATT
TACTCATGTTGAACCTAGTTTTATTTACAGTATATTTGTATGCATAGAAGATGGAGGTCC
ACCAAAGTGTTAATTATGCTTAGTTGTAGGTCAGGTATAGCTAACTTTCCTTTTTTAATA
TATATATTTACATTTGTGTTTCCTTTATAATTTATGGCATAGATTGCCACGATTTTCTTA
AGTATACTTTTATAATCAGAAAAATGATATTAAGGACTCATTTTAAGTACACTAAATCAA
ATATTAGAAGGCTTCTTTATTTTAAGCTAATTGTGAGGATTATTTGTCATTTAAAACTTT
TGCTTCTACTTATTACCCTGAAGTATCTTTGTGGTGCTTATGTTTTTCACAGACTGTATA
AATTGATATACTCTCCCGCCCCATGGTAATGTTGCTACACATAAGCTCTAATAATTATCA
TTTTTAATGTTTTAAGATTAATTCAACTAAGTTTTAAAAATAATCCATTGGTTACATACA
TAAGAAAGTACTGTATACAGATTCCCCTGACTTATAATGGTTCGACTTAAGATTTTTTCA
ACTTTACCATGATGTGAAAGCCATATGAATTCATTGTGCTCCTCGATTTATGATGGGACT
ACATCCAGGTGAAGTCATTGTAAATTGGAATTGTTGTAAGTTCAAAAGTCACTTTTTGAT
TTAAAATACGTGTAACTTACACTGGGTTTATCAGGATGTAACATCACAAGTCGTGGAGCA
TCTGTATTTCGGTCATTTAATGGATGATATCTGACTGAAGGGAGAAAATGAATATAAAAG
GCATGAAAACAGGAATAGAAAAGGCATGTTTAAAGTTCTCAGCGCAGGGCTGATAACTCT
AGCTGCTCTCTGGAGGTGGTGTTAGGATTTTGTTGTTTTTTAGTTAAGGATTTCCCACTG
GAAAAATGTAGGTCTGCTTATTACAGTATGTTTTCAAATTTCTAATACCCTGCCTTTCCC
ACTGGGACCTTATTTGAAATAGTTGAGTTAACTTTAGTCTTGTGTCAAATAGTACTCTTT
GAAGTCATGGCTGATGTTTATTGAGAGTTGACTGTACTAGTTTCAGCTTTTTTTTTTTTT
TTTTTTTGAGACAGAGTCTCACTCTGTTGCCAGATTGGAGTGCAGTAGTGTGATCTTGGC
TCACTGCAACCTCCTCCTCCTTGCAACCTCTGCCTCCGCCTCCCTGGTTCAAATGATTCT
CCTGCCTTAGCCTCCCGAGTAGCTGGGTCTACAGGGACATGCCACCACGCCCAGCTAATT
TTTGTATTTTTAGTAGAGACGGGTTTTCACCATGTTGGCCGGGATGGTCTCGATCTCTTG
ACCTCGTGATTCACCCGCCTCGGCCTCCCAAAGTGCTGGGATTACAGGTATGAGCCACCA
TGCCCGTCCTTTTTTTGAGACAGGGTCTTGCTCTGTCGCCCAAGCTGGAGTGTGGTGGCG
TGATCTTGGCTCACTGCAACCTCTGCCTCCCAGGTTTGAGCCGTTCTTGTGCCTCAGCCT
CTTGAGTAGCTGGGATTATAGGCGCATGCTATGACACCCAGCTAATTTTTGTATTTTTTT
TGTAGAGACGGAGTTTTGCTATGTTGGCCAGGGTGGTGTCCTTGACCTCAAGTGATCTGC
CTGGCTTGGCCTCCCAAAGTGGTGGGATTACGGGTATGAGCCACCACACTTGGCCTTAGA
CTTTCTCTTACTTTATATATATTTAATCTCAGTCCTTAAAATAACTGGGTAGATAGGAAG
AAACTGAGACAGAAATTAGGTAAATAAGGCCCAAGGCAGTTAAATAGGATAAAAGCCTGG
ACTGACTGTGCTTTTAACTCACTACCCTGTACTGTGAAAATTTACCTATATTAATTATAG
AATCTTAAAATTCTGGACTGAGTGTAAGCAGTATGATGTAGGTAATGACTTTAGAATTCA
ACTGCAGTAAGTAGGTTAATATTGTAAGAACTAATTTGCTTTTCTGAAGTAATTTAAAAT
GTGTGAATATCCTATATGAGGGGCCTTAAATATAACTCACTCAGTTCTTTCTCAAAGAAA
ATGAGAAAAGGAATGGTAAATGTTAACTTGCAGGCCTCTTTTTTTTGTTTTTTGTTTTTT
GACTCTTAAAGCACTTTACTTATTTTTAAAATTTAATTAATTTTTTGTAGAGATGGGGTC
TCACTTTGTTGCCCAGGCTGGTCTTGAACTCCTAGGTTCAAGTGATCCTCTTGCCTCAGC

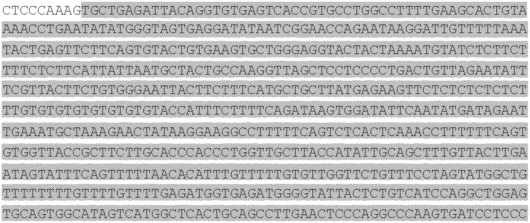

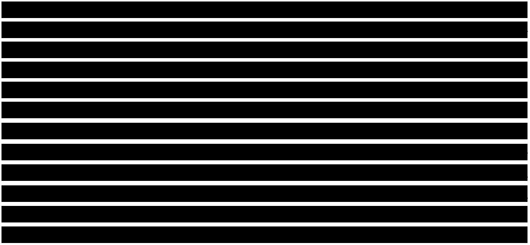

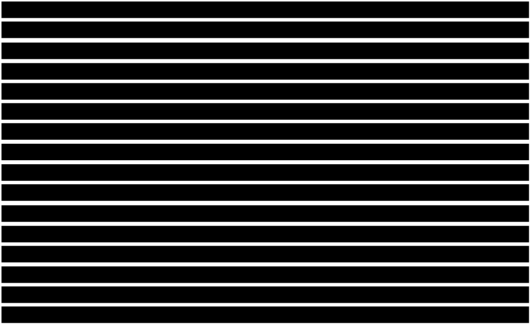

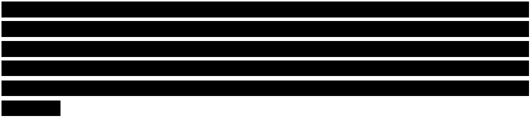

Transcribed seq : EST support for target seq
TCAGCCTCCCAAAGTGCTGAGATTACAGGTGTGAGTCACCGTGCCTGGCCTTTTGAAGCA
CTGTAAAACCTGAATATATGGGTAGTGAGGATATAATCGGAACCAGAATAAGGATTGTTT
TTAAATACTGAGTTCTTCAGTGTACTGTGAAGTGCTGGGAGGTACTACTAAAATGTATCT
CTTCTTTTCTCTTCATTATTAATGCTACTGCCAAGGTTAGCTCCTCCCCTGACTGTTAGA
ATATTTCGTTACTTCTGTGGGAATTACTTCTTTCATGCTGCTTATGAGAAGTTGTGTGTG
TGTGTTTGTGTGTGTGTGTGTGTACCATTTCTTTTCAGATAAGTGGATATTCAATATGAT
AGAATTGAAATGCTAAAGAACTATAAGGAAGGCCTTTTTCAGTCTCACTCAAACCTTTTT
TCAGTGTGGTTACCGGTTCTTGCACCCACCCTGGTTGCTTACCATATTGCAGCTTTGTTA
CTTGAATAGTATTTCAGTTTTTAACACATTTGTTTTTGTGTTGGTTCTGTTTCCTAGTAT
GGCTGTTTTTTTTGTTTTGTTTTGAGATGGTGAGATGGGGTATTACTCTGTCATCCAGGC
TGGAGTGCAGTGGCATAGTCATGGCTCACTGCAGCCTTGAACTCCCAGGCCCAAGTGATC
CTCCCACCTCAACCTCATGAGTAGCTGGGACCACAGGTGTGCACCCCATGCCCAACTAAT
TATTTTTGTAGAGATAGGATCTCACTGTGTTGCCCGGGCTGGTCTCAAACTCCTGGCCTC
TAGTGATCCTCCCGCTTTGGACTCCCAAAGTGCTGGGATTATAGGTCTGAGCCACCATGC
CCAGTCAGCGTTTATCAGATTACTTACCATTACTACTTTGTCCTGGGGAAATCCTCTTAA
TCTTTAAAGGCGCAATCCAAAATCATAATGTTCCCGTGTTACTTACTGTTACTTTTTTTT
TTTTCTCCTATAGTGGTTTGATGATAAGAACCCAATTTGGGCCGGGCGCGGTTGCTCACG
CCTGTAATCCCAACACTTTGGGAGGCTAAGCCAGATGGATCACCTGAGGTCGGGAGTTTG
AGACCATCCTGACCAACATAAAGAAGTCCTATCTCTACTAAAAACACAAAATTAGCTGGG
CGTGGTGGTGCATGCCTGTATTCCAGCTACTTGGGAGGCTGAGGCAGAAGAATCGCTTGA
ACCCAGGAGGCGGAGGTTGCAGTGAGCCGAGATCGCGCCATTGCACTCCAGCCTGGGCAA
CAAGAGCGAAATTCCGTCTAAAAAAAAAAAAAAAAATTGGAGTTTTACAGATAACCACAT
CTTATTCTGGGAAAGGATTTGAAGCAAGTTGGGTTTTATATTTGGCTGTACTTGTCCTCT
TCAGCAGTATAATAAGCCCCTTAAGGCTGAAGTAACCTTATTCCTATTGTTTAGTAGCTA
ATAGCATGCTTTTGATATGCTTATGATCATACTAATAATTTAATATTTGAATTGTATGGA
AGTACAATTCAGTATCATTTTACATATGGTATATTGTGATGCTGTATCATATTTTATGTT
ACGGTTTATAAGAAAAGCTCCTAGGTATAAAATGCTACATAGCAGGAACTTGGTTTTTCA
ATGTTATTATTTCCTACTGTTTTTGACGTAACGGCAATAAAATTTGTTTGAACCAAAATG
GACTAACAATTATTTGTACAACTCAGTATTGTCTAAATATCATATTGTTAAATCTAGGTT
TCTTGAATTCTCCATCAAGCCTGGTCATGTCATGTAGCATTTGGTGTCTCACCATGCCCA
ACAGATATTTTGTGGGAGGATGGAGTTGATCTTCCTCATGTTAAAAGATTGAAGGGAGTG
TTCTGACTTAATTGATAACAGTCTTTCATAACTTCACAAATTTTTGAGAATGACCCAAGG
CTAACTGTGGGAAAAATTCACATAAAAACATAGCCTATCTATGAGGAGCAAAACTATATT
TCAGTTGTGGGCTTTACATTTCATTTAACCCTCTTAACTGTCCTGTGAAATGGGTTACAG
CCTTATTTTATAGATGAGGAAGCTGAAGTTTAAGGGATTTGCATACGGTCACGTAACTAG
TGAGTTGTGCAGCTAGGGTTAGAATAAACAGATTTATTTTTTTTTTTTCTTAGAAACAGC
AATTAACAATGTGACTCCTAATCAAAAGAAAAGAGATGTCCTTGGGGCTTAAAGTACTAT
GGTGGGAGTCTTGGACTGAGTAGGTTTGAAAATACAATTTTATGATCGTGGAGTACTAGG
ATTTAGTCATTTTGATGCAGAGCATTTCCTGATCAACTGCTGTTGTGGAGTGTACTGTCC
AATAGAATTCTCTACAATTAAGGAAATGTTCTGTATCTCAAGAGATTGTTCTTAATGGTG
GCCAGTAGTCATGTGACCGTTGAGCATTTGAAATGTGGCTAGTGCTACTGAAGAATGGAA
TTGTAAATTGCTTTTAATCTAAATTTTGCCTGTGATATTATTGGCTGTGGGTTTGCCAAA
ATTTGTTTTTTTAAAGAGGAAAAGATAACGGACTGTTGGCTGCTTTATTGGACAGCACAG
CTAGCATATAGATGCAGATAGGTAGTATAACTTGTTTGTAGTTTAATATAAATGTTGTAT
TTTGTAATTAG
Transcribed sequence is
CTCTGCTTCTGAGACCCTCCTGTTACTGTTATCATCGTTCCCTAGCCTGGCTCTGCCTTT
CTCAGCAGCCCACATTCCATGGATGGGAGCAGGGGGGCAGGGACCCAAAGGAGGGAAATG
GCTGTGGGTGGTGTGAAGGCCCCCCAGCCCTCAGGAAGGTGGGGCAAGAGACCACTGAGC
ACAAGGGATCTTGCCCACCTCCTCTTTGACTCTGTGGATTATCCATCCATCTGCTCACTG
TGAAGATGGAGAGGCAGTGCCCTAAGGCTGTTCAATAGCTTTTCCATATTTTTTCAACAT
TGAAAAAATAATTTTTAAAAACTGTGATTTTTTTAAAAAATCATTTGGCTGGAGGGAAGG
GAAAAGGGAAACACCAAAAGCTGTACCATGATGAACTGGAGATATTTAACTGGGGCACTT
TCCAGACCAAGACAAACAAATTCCTTTCTGGACTCTAAAGCAGCCGAATCTTGAGACTGT
CAATGACAGAAAGCTGAAGAGAGGCCTCTATTTCTTCCTTTTTCCTTTCTTCTGTCTAAA
AACTCTCTCTTGTTCCCCTTTTCCAGCTTCCCTTGGACTACTGCCCCAATGGCCCCTTGG
ACTCGCGTTTCATGTATGCGAGCACACACACACACAAACTTGCAAAATACCGTTTTTCTT
AAGGATTGTGGGACCGAATAATATCACGTGCCTTCATCTTTTCCTTTTATAGTTAGATGA
ACCTCTTCCTCTTTACAATTTTTTTAAAAAGTGATAGGGGAGGTTGATGTGTTAGTGGAA
GATTTGGGCATCGTTTGAGAAGTAACTTTTGTTTAACACATTCCCCCTAAACATTGAACA
CAAACATTTCAACCCCTTCATGACACTCTTTGGACATTTAAAGCATTGAGTAACCATGTA
CATGACAGCCTAAATCCGTTTGATTTCAGAGCATTTCCTGAACATTGTATTTCATAGACT
TCTCTGATTTTTTCAAAAATGAGGTGAGCAATGGCAAGCAGCCTTGTTCTCCCAATTTGG
TGCTTTTGCTTTTGGTGTGGGGTGGGCATGGGGGGTTGGGGGTGGTGTGGGTGTGTTTAG
AAAAAAGATGCATTCCTGAAGATCTCTGGTGCTGAAGGGCCTCGAGTTCCTTTCAGAGAC
TGTATTTGACACACTTTAGGTACACACAAACGAATGGTATCACATGCAATATTTTAATGG
AGCAATGGGAGAGGCTCTTTGAAATGGGGTTTGCATCTTTTTGTAACATTTTGATTTCTC
TGGTGCCTTATTCCTACTTGATGCTGGCACTCACATACCCACAAGAAGCTGACACAGAAG
TCAGCCTTAGGCGTGGGGACATATGGGTGATGTTTGAGCATGCAGGGGCCATGGGGAGTT
TGGTGTCAGTTGGTGGAGAAGGGACTAGATGGCATCTCTTAGCCGAGGCCAACAGGAACT
GCACAAGTCCATTATAGTCAAAGTTAGCAATTTTGATACGTAAACACAATACTTCATTCT
TCCTCATCTGAGCTTTCCTTCCTTCTTCCTTTTCTATCTCTACCTTCTCATAAAGGTGCT
GCTGCTGCTGCTAAGGTGCCCGGAGTCCAGAATGTCCATTAATCACTCAGGCACGAGCCT
GGCACTGCCACGTCAGCCCCCAGCATGACCAAACCCAGGTTTCTCTTGCTTGGGGCTGAG
AACTGTCAGATTTTTCTCATCAAAAATGTTTTCCAAGGAATCAGTGGATTACAGTTTTTC
TGCATTGAAAATGCACTTTAAAAAATAAATTAAAGCTCCAGACTGTTTAAAATATACAGA
GGGAGCAGGGGAAAGTTAAGCATGTGCTAGTGTCTGAACCCAGTTCAGTTTATCTCCAGT
TGAAACGATATACACTATATTATGTATAAATGTATACACACTTCCTATATGTATCCACAT
ATATATAGTGTATATATTATACATGTATAGGTGTGTATATGTGCATATATACACACATGC
ACATAACAAAATCAGATGCTCATTACAAATCCAGATGCTCATTACAAAACCAGATGCTAC
ACAAACAGCAGCAGAGGAAACAAGGTTGGACTCTTGCAACAGATCACAAAAAATAAAAAC
AGCTACTTGCAGTGACTTTGGTCATTTCTGTATGTTCATAAAGAATGGATTGTAACAAGG
AAAAAAAGGAACAGTGTTAGTGAAAAAGGAAAAATGGGCGAAACCATCTTGATCCGATGC
GAATGCAGTAATGTTCTATATACCATTTCATCAGTTATTTCTTTTAGTCATGTTGATTTG
ATTTCAGTTTCTGGCTATGAAAAACATTTTTAAACTCGTCACCCACAACAAACTGAACAA
AACTACTACAGTGAAAGCCCTTTTCAGTGAAAGATGTCAGAAACCTCAAAACCTTTGGCC
TGACTCAGAACTACCATGTGAAAATCAGTACTCTCTTAATGTTTGAAATAAAAACTGAAA
AAAAAAACAAAAAAACAAAAAACCTTTTTTGAAGCACCTTAACGTGGCCATCCATTTGAG
AAGTGGGTGCCACTTTTTTCTTTGAGCACCTTATTGATGTGTTTGCTATCTGCTGTCTTT
CTGTTACCTGTTGGCTGAATGGCTAGCTGTTAACATATACATGTGCACAGAAGAGATATC
TGGGCATGTATGTTCTCAATGAAGTTTACTGTGGTGACTGCTGAAAGGTGAACCCATTTC
CTGATTTTCCCGCCGCAGTGTTGTGATAAGATTCGAAGAAACCTTTTTCCCTGCACAGAA
ATGTTTCTTATCACATTGTATCTTAGTATGGAAAGGAATATGGTCCCTTTTTTGCAATTG
CTACTGTGTACACACACACACACACACACACACACACACACACACACTGTATGTTTAGAC
CTAAAATACACACACCCACGCACACACTGTATGTTTATGTGACCTAAAACATACACACAT
GCACACACACATACATATCCATTCATTCATTCATTCAAGTGGTGTTTCCAGTGTCTGTGT
GTCACTGTTTATGCAGTTTCCATTTCCCAGTGAATTATGAGTGGAGGGCAACTTTTCTAA
CCAGATTGTCTTTTCAGAACAAAGACCTGGGAATTGAGGAAGAGTTTGGAAAGAGGGAGA
GGCAAGGAAAGAGAGCTTTAAATTGAAAGGTTAATTTCCTAAGAGGAACCTGGGCTGAAT
GACTGCAGTGTTATACCCTCCAATCTTTGCAGGTGGGCATGGAACACTGCTTGTATCACT
CTGTGCACGGTATAAATCCATATATCCACAAAAACACACATCCATCCATCAACATATACA
TGGTTTGGGATGAGCAGGTCAATAGTTTTGAGAGGGAGTTTGTTCCTTTTTTTTTCTCAT
TATACTCTTAAATTGTTGTCAGTTATCAAACAAACAAACAGAAAAATTGTTTGGAAAAAC
CTTGCATACGCCTTTTCTATCAAGTGCTTTAAAATATAGACTAAATACACACATCCTGCC
AGTTTTTTCTTACAGTGACAGTATCCTTACCTGCCATTTAATATTAGCCTCGTATTTTTC
TCACGTATATTTACCTGTGACTTGTATTTGTTATTTAAACAGGAAAAAAAACATTCAAAA
AAAGAAAAATTAACTGTAGCGCTTCATTATACTATTATATTATTATTATTATTGTGACAT
TTTGGAATACTGTGAAGTTTTATCTCTTGCATATACTTTATACGGAAGTATTACGCCTTA
AAAATACGAAAATAAATTTTACAAGGTTTCTGTTTTGTGTGGAAGAGTAATTGATGTTGC
TAAGAATGATGTTTGTTTTTTTGGGGTTTTTGTTGTTTTTTTTTTAAATGTTACCAGCAC
TTTTTTTGTAAGTTTCACTTTCCGAGGTATTGTACAAGTTCACACTGTTTGTGAAGTTTG
AATATGAAGGAATAATTAAAAAAAAAAAAACTCTT
MOUSE TRANSCRIBED SEQ (Homologous to Human
213158_at transcribed seq)
AAATCTTAGAAGCAATCGGGGTTGACAGCGCTTTCGTAATTACTAATGAGAGGATCTTGT
GCTACCGGAAGAGCAATAGACTGTGTGGCGACTCAAACAAGTGTGGGGATGCTGAGGGGC
TCCTCCAGAGTCCCGGATGACAGCTCTTGGAAACCCTTGTTTGCTAAGAATCACAGCCCT
TGTAAACACCTAATGTTGAGTTTCTTTGAACACTGTCCCACCTGAGGGGATTCGTTTGGA
AAGCTTCCATTTCAGGCCTCTTTAACAGAGTATCAATCTGATGCTTTCTCCTTCCTCCTT
ATGATAGGTCTCATTCTACTTTCCCATGTCAGAGTTTCTTTTTATATATACAAAAGTGCC
AGCCTTGCTAGTTTAACCCTACAGAGACCATTCAGAACTAACTTAAGCAGCAACTTAGGA
GAACTCAAAGCATTATCTGTATTTCAAGCAGGCTCCTGAATCAGATCTCATAGCAGATGC
CTGGGAATGCGTGGTGGGAAAGCACTAACAGGACATGGAGACACCCAACCAAAGCTATGA
GAGGAAACAGTTGACCTTTAAAACAGTCTCACCTTAACTTTCCTTGAGGCATTGGGGACA
AGTTTTTCTTGAAACTTGCATATCCACTCCAGTTCCTTCACCAAAGATTTTCTTCTCCAG
AGCCCAGCCTCCTTTCTCCCAGGCAGAACCATAACAGGCCTGAGGGTGTCCTTGCAGTGG
TCCACAGAGTTCACCTTCTGTTCACAGGGGTATTTACAGACCTTATAGTAGAAGGGTTTC
CAAACAGTCTGTATGGAAAACATACACAGTACTACTTGGCACTGCGAGCTTTGTGAGACT
CATCTGTTGCCTGGAGGCTTGTAGTCAGAAATATCCATGGAAGGGAGAGTGCGAAGTCAT
TTAGAGCCAAACAGGACCGCTGGTGAGAGGATCATTGGGCAGTATGAGTCAAGAGCAGAT
CAAGGCTCCGTGTGCCCAGGGCCAATGGCAGTGGCCTATGAGGATGTTAGACAACACATC
AATGGAGTCACATTCTGAGAAGCTAAAGTGTGGGCTTTGCTGTAATGGCTGACATTGTTG
AAATGTTCGTGCCACAGCAAGGGAACTACTTGGAAGTAGACCTGTTGTGATAGTGCCTTC
TTGTTGTAGCAAGTCATTTATTCAGTTAGGCTTTTCTGGACCATTGCCCCCATCTTCTGA
AGAGGTCTGAGATGAAGGGATAGGACACTGCCCCTGAAATGCTGTGATTTGAAGATATTT
GCACTAGATTCTATCCTCTCCTTTAAACTGGAGCAACTGAATGAGAGGGGAAAAATTAAC
AAGGACAGCTCAAAATGAAAAGAAACCCAAAGTAATGTGTTCTGATAACATTATCTCCCC
TCACTGCTACATCTTTCCTCCCCCCTTCCTCCCTTCCCTCCTAGATCTACTTTTTTTTCC
TTCCTCTTAAAGGAAACTTCCATTTTCTTATTACCAAATCCAACAATTACTTCTCTTTGT
TTCTCCCCAGTACTGAATCATAAGCTTATTAATCACTCATGAGCTAGGAATATCTAGTAA
AGAGCCTCTGCCTTGACAGTGTTGCTGGCCTTCTCTGTCCATCACGGGTGAACAACGAGG
GGTAATAGGGAGACTAGACTGGCCCAGCTCTTATGGAAGCCAGAGTCTGGATTTCACACC
TATAAGGAGATGACACCTATTTACCCAGAACACATAGTCTGCAGCTCATCTTAAAAGACG
CTTAGGAACAAAAGGAAGTTCCTGTGTTACAGCAAACAGATGCGGTAGTACCCAAAGCTT
ACCTGTCTCTTCTCTCTCCTCTCTTCCGTCTTACTGCCATGTCCTCTCAACGAGACTTAA
ACTTCATCTCATGAATGGCACCAGAAGAACTATTTGACTCCTTGGCTTCTCTCTTTTTCA
GTAGGCTGGTAGCTCATTCAAAATTAAAACCAAGCAAATACTATTAGTGGCTAGCCCCCT
GAGGGCTGAACAATTTCCCAAGTGTCTTGATGATCCCAATATCTTGATAATCAACTCTGA
TAACTTGGAAGTTTTGGCTGGCTCAGACATCTGTCAACTTTATTTTCATTTTGTCTCCAT
TTCCATTTGAATCTTAAGTGAGAGTGGAAAGGTAGAATCATGGGAAAGATTGTGAGGCTG
CAATTCTAGGGTAGAGTTTGTCAGAAGTTTGTATTATCCCAAATAGAAATTTCTATACTT
ACTTTCAATTTAATGTTACCCTGAATATAATTTCTATTACATTTATTGTTATTTTTATAA
AAATAGAGTTCAATTACTATGTCTAGTTGAGTGCTCTCTTTTCTATTTTCCCACATGGAT
GCAGTACCAACCTGTTACCTAAATATCTTTTTATTATATTGTTAATATGTAATTCTACTG
TAGACCAAAAATATAAAAACAAATTTGCTCATTTTAAACATATACAGACTCTAATGAGTA
AAGATGAGGAGAAAAGACCAGAGAGCAGTGGTTGACTATGTTGTTAGAAATCAAAGAGTA
GCCTTACCTATTTTTAACCAGTGCTTGCCGTCACACCATAGTTAGGACTATGTTAGCATG
GCTTCTTCATGCTTACGTTCTGCAAGCCTTGTCTGTCTGTTTCCTTTGATGTGTTCGAGG
TTGCACAATGATGCTATTGTTTTTTTCTTTTGGTAATGCCTGATTTTATTATAATGTACT
TTATCAGTCATTTCCTTTAGAAGAATGAGGGGGAAAGTTTTATTTCTTCTTTTAATTTAA
ATTTTGTTTAATGCACTGGAAATAAAATTGGACACATTTCACTGTTTAAAAATCAGAAAC
GAAACAAAACAAAACCCCGAAGAAAAAACCAGCAAACAAGTAAGTAATAGGATACACACA
CATACAAAAAAGCTATGAAAAATATTCTGTTCATACAAAATATAGGCTATATCTCACATG
AGAGATAAATACTGTCAAGTAATAAAAAGACATTGTCAACTACAGTGCTGAAAACTATAA
GAGGAACCTAGGTGTACAGTGTGTGGGGAAAACTACGAATCCTTTCTGAGGCGAGATCTT
TCCATTGTTCCAATAAAAACCTAAGCAAGTTGAATGTGGAAGTCGGTAAGTAGGGAGCAC
CCCGCCTTCTTTACACCAGCGGACCTCTGGGTTACTTTCTACCATGGGTCTCAGCCACAT
ACACATACACACGCACGCACTCATGTGCACACACTCAATACTTGAGAAGGATTTGTGAAA
ATGTACATACCCAGTACACAGATGTACACAGTGCTCTGACAGCCCTCAAGCTCTTCTGAG
GCTTAGCAGTGATGGGTCCACAACATGGAATACTGAAAGGGATTCACTGAGATCTACGTG
TGCTAATAAAGTGCTTGAAGCCAGCCTGGTCTCTTCCCCAGCATCCCCTAGTCCAAGGCC
AGCTGCCACACACACATGGACAGAGAAAGGCGAGACACCGGTTACTTCTCCTAGCCAACT
GGCTCATTATTATTTGCTGAATATTTGCTGGATTTTTCTGGTTTTGTTCTGTTTTAGAAT
GGGGTGGGAGTGGATGTTATGTCACAATCCTAATACAGTAAAGTTTTGCATCTTCCATAT
CTTATGCAAAAACAGACATTTAAATCAATAAATAGTTGTGCCCTAGACTGAAAGTTAATG
TTTAGGAGAGGGAAAAATTGTTGGAATTTTTTCTACATTTTTTTGTGAAGAATCTTTTTT
GGAAAGGAAGGATACATATTTTTGTTGTGTAATATTTTCTATTTTTGAATGCATTTTATT
GGTACAAGACTGTTTTTTTGGTGAAGACATTATTTAAAAAAAGAAAAAAAGAAAAAAACT
AATCGAAAAGTTTGCCCTTAAGGATATGCTGCAGTTTTGAGATTAAAAAATAATAACTGA
TTCAAGATGCGTGTTAAAAGTTGGGATTATATTGTTGTTTTTGTAATTGTTACAAGAAGA
AGTTTGTACCCACTGCTGTTTATTTTGTTTCAGATGAGTAAGTAAAGGGATTGTTCTTGT
TTTATTCTTTTTTTAGAGAAAAAAGCTATTTATGAAATGTCAAAAACACTGGACTGTGAG
TTTAAGTGTGGAAGCATTTTACCACCCTGTGTCTTCAACCAATTATGGGAAACCTTTTCT
CTCCCCCCCTGCCTTAGCCTTGCCAAATGAGGAAAACGTAACAGCTCTCAGATGACGGAA
GTCACCGAAGCCCTGCTTTAATTTTTATGGTCTGAAAAAGTCGGAAAACCAAAGTTAAAT
TTGTTTCTGAAATCCCGCTGTCTATAGCCCCTTTTTTGTACAACACAGCCGGCTGGCTCT
GCCTCTCTATCTTGGATCATTGCCTTCTTAGGAACGTGGGGCCAGCTCTGCCAAGAGGCG
TGAAGGTGGCGAGGTCACAGGAAGTGAGGTGTGAGGGGGACCCCTAGGGCCCCGGAGCTT
CTCCATCCAGAGGCGAGGCTGCCAAGAGCACACACAGCTAACAGTGCCTGGCGGGGTCGC
CCCTGTCCCCCTCACCTTCTGCTTCGAAGACCCTCCAGTTACCGTGGCTCTGCCTTTCTC
AGCAGCCCACGTTCCGTGGATGGGAGGGGGTGGGATCCAAGCAGAAAACACGGCTGTGGG
CGCTGCGAAGGCCCCGGCCCTCAGGAGGTAAAGCAAGGGACCACTCAGCACAAGGGCTCT
TGCTGCCCGCCTCCTCTTTGACTCTGTGGATCGTCCATCCATCTGCTCACTGTGAAGATG
GAGAGGCAGTGCGCCCTGAGGCTGTTCAATAGCTTTTCCATATTTTTTCAACATTGAAAA
AATAATTTTTAAAAACTGTGATATTTAAAAAAAAAAAAATCATTTGGCTGGAGGGAAGGG
AAAAGGGAAACACCAAAAGCTGTAACATGATTAACTGGAGATATTTATAACTGGGGCACT
TTCCAGACCAAGACAAATGAATTGTTTTCTGGACCCGAAAGCAGCCAAATTTTAAGACTG
TCAGTGACAAAAAGCTGAAGAGAGGCCTCCATTTCTCCTCCTTTCTTCTTTCTGTCCCAA
ATTCTCTCATTTTCTCTTCTAGCTTCTCTTGGTAACTGTCCAATGGACTTCATACTTCAT
GCAAAATCCCGCGCATGCACGCGAGCGCGCACGCATGCGCGTGTACACACACACACACAC
ACACACACACACACACACACACACACAAGCAAAAAAAAAAACTATTTTTCTTAAGGATTG
TGGGACTAAATTTAAAGTCATGTGCCTTCATTTTTTTCCCTTTTATAGTTAAATGAACCT
CTTCCTTTTTTACAATGTGTTGGGTTTTGTTTTGTTTTTAGTAGAAGGGGAAGGTTAAAG
TGTTTGTGGAAGAGAGGATTTTTAGGCATCAACTGGGAGATTTTTTTAGCATATTCCCCC
ACTAAATATTAAACACAAACATCTCAATCCCTCCACGTGTCACTGTGCACACTTAGAGCA
TCAAGGAATCAGAATCCGACAGCCTAATCCACTTGATTTTAGAGAAGTTCCTGAAATTTC
TATTTCCTAGACTTTTTTATTGTTCTTATTTTATCACAGTGAGGTGAGCAAGGCAAGTTG
CCTCGTTCTCCCAACTCGGTGCTTCTGCTTGTGGGGTGGGGGTGGGGCGGTATAGACAAG
GGTGCACTCCTAAAGCTCTCTGGTGCTGAAGGGCCTCAAGGTTGAGTTTCTTTCAGAAAA
TGTGTATGGCACACTCTCAAGTGCACACGTGAACGGTGTCATGCGCACTATTTTTAAAGG
ACAAGGGAAGGGGCTCTGAAGTGGGTTTTGCTTTCTCTCATGACATTTGATTTCCCTGGT
GCCTTATTCCTATTCTATGCTGGCACTCACATGCCCACAGGAACACACGCTGATGTCAGC
CCCAGGAGTGAGGACCTCTAGGTGACAGTTGAGCATGTGGGGACCATCGGATATTGGGGT
CAGTTGGTAGGGGAGGAACTAGATGGCTGAAAATACACAGGGACTGCACAAGCCCATCAC
AGTCAAGATTAGTAATGCTCATATGTGAGTATGTGCAATACATGCACACACAAACACACA
CACAGACACACACAGAGATGCACACACAAACACCAAATACACTCTTCTTCCTCTGAACAT
TGCTTCCTTCTTCATTTCCTGTCTTTGCCTTCTCATAAAGGTGCTGCTTGCTGCTGCTGC
TGAGGTGCCCGGAGTCCAGAATGCCCAGTAATCACTCAGGCACAAGCCTGGCACTGCCAC
GTTCAGTCCTTGGCAAGACCAAACCCTGGTTTCTCTTGCCTGGGGCTGAAAACCGTCAGA
TTTTTCTCATCAAAAAAAAAAAAAAAAAGTTATCCAAGGAATCAGTGGATTATAGTTACT
CTGCATTAAAAATGCACTTTAAAAATAAATAAAAGCTCCAGACTGTTTAAAACACACAGA
GGGAACAGGAGAAAGATAAACGTGCTAGTGTCTGAACCCAGTTCAGCATATCTCCAGTTG
AAACAGTATACACTATATTATGTATAAATGTATACACACTTCTATATATGTCCACATATA
TGCGGTGTGTGTATTATACAGGTATAGGTGTGTGTGCACGCACACAGGTGCACATAGCAT
ATCAAGTGTTCATTACAAATCCAGATGCTCATTTCACAAACAGCAGCAGAGGAAACAAGG
TTGGACTCTTGCAGCAGATCACAAAACAATAAAAACAGCCACTTGCGGTGACGCTGGTCA
CTGCTGTGTGTTCATAAGGAATGGATTGTAACAAAGGAAAACAAGGAGCAGTGTTAGCAA
TTGAGGAAAACTGGGACAGACCATCTTGATCCAATGGGAATGCAGTAATGTTCTCTACCA
TTTCATCCGTTCTTTCTGTTAGTCGTGACGATTTGATTTTCATTTTTGCCTATTAAAAAT
GGTTTAGATTCAAGTGACCACATCCAAGTGAACAAAACAACCACAGTGAAAGTCCTTTTC
AGTAGGAAGATGTCAGAAAACTCAAAACCCTTGGCCTGGCTCAGAACTACCATGTGCAAA
CCAGAACTCTCTCAACGTTTGAAATAAAAACTTTAAAACTCTTTTTGAAGCACCTTAACG
TGGCCATCCATTTGACAAGTGGGTGCCACCTTTTTCTTTGAGCACCTTATTGACGTATTT
TGCTATCTGCTGTCTTCTGTTACTGTTGGCTGAATAGCTAGCTGTTAACACACACACATG
TGCACAGACCAGACATCTGAGCATGCGTGTTCTCAATGACGTTTACCGTGGTGACTGCTG
GAAGGTGAACTCATTTTCTGATTTGCCCACCACAGTGTTGTGATAAGACTCGAAGAAACC
CTGCCCTGCACGGAAAAATGTCCCTTATCACGTTGTATATTAGGGTGGGAAGGAATATGG
TCCCCTTTTTGCAATTGCTACTGTGTATACATACACATGCACACACACACACACACACAC
ACACACACACACACACACACACACACACACTGTATATTCAGACATGATGTACACACACAA
ACATAACTCATTTGTCCAAGTGATATTTCAGATGTTTCTGTGGGTGTCACACACCATGTG
CAGTTTTCCACTTCCCAGAGAATTTTGAGTGGAGGGTAACTTTTCAGACTGATGAACGGG
GCACTGAGGAAGAGTTTGAAGTGGGAGGCAAGAAAGGAGAGAGCATTAAGTCAAAAGAAT
AATTTCCCAAGAGAAGCTGGAGGAATGGCTGTCCTTGCAGGTGGGTGTGGAACACTGCTG
TCTCAGTCTGCACTGTAGAAATCCATGCACACATCAACACACACACACACACACACACAC
ACACACATACACACATCCCCCCACAGGGGCGTGGTCTGGGATGAGCAGGTCAATAGTTTT
GAGAGGGAGTTTGTTCCTTTTTTTTCTCTCATTATACTCTTGTCAGTTATTAAACAAACA
AACAGAAAAAAATTGTTTTGAAAAACCTTGCGTACGCCTTTTCTATCAAGTGCTTTAAAA
TATAGACTAAATACACACATCCTGCCAGTTTTTCTTACAGTGACAGTACCCTTACCTGCC
ATTTAATATTAGCCTCGTATTTTTCTCACGTATATTTACCTGTGACTTGTATTTGTTATT
TAAACAGGAAAAAATTTCAAAAAAAAGAAAAATTAACTGTAGCGCTTCATTATACTATTA
TATTATTATTATTGTGACATTTTGGAATACTGTGAAGTTTTATCTCTTGCATATACTTTA
TACAGAAGTATTACGCCTTAAAAATACGAAAATAAATTTTACAAGGTTTCTGTTTTGTGT
GGAAGAGTAATTGATGTTGCTAAGAATGATGTTTGTTTTTTGGGGTTTTTGTTGTTTTTT
TTTTTAAATGTTACCAGCACTTTTTTTGTAAGTTTCACTTTCTGAGGTATTGTACAAGTT
CACACTGTTTGTGAAGTTTGAATATGAAGGAATAATTAA

We claim:

1. An array comprising a substrate having a plurality of addresses, each address comprising a distinct polynucleotide probe affixed thereto, wherein at least 10% of the plurality of addresses have affixed thereto polynucleotide probes that hybridize under stringent conditions to markers selected from the group consisting of the markers indicated in Table 1a and Table 1b; and wherein at least 10 of the markers hybridize to the array.

2. A method for providing a diagnosis, prognosis, or assessment of asthma in a patient comprising the steps of:
(a) detecting a level of expression of at least one marker that is differentially expressed in asthma;
(b) comparing the level of expression of the at least one marker in the patient to a reference expression level of the at least one marker; and
(c) providing a diagnosis, prognosis, or assessment of the patient's asthma based on the comparison done in step (b);
wherein the at least one marker is selected from the group consisting of the markers indicated in Table 1a and Table 1b.

3. The method of claim 2 wherein the reference expression level is selected from a numerical threshold, a level indicative of an asthma state, a level in the same patient at a different time point, a level in the same patient before a treatment regimen, or a level in the same patient during a treatment regimen.

4. A method for evaluating the effectiveness of an asthma treatment in a patient, the method comprising:
(a) detecting a level of expression of at least one marker in a sample derived from the patient during the course of treatment of the patient; and
(b) comparing the level of expression of the at least one marker in the patient to a reference level of expression of the at least one marker;
wherein the difference between the detected level of expression of the at least one marker in the patient and the reference level of expression of the at least one marker is indicative of the effectiveness of the treatment of the patient's asthma; and
wherein the at least one marker is selected from the group consisting of the markers indicated in Table 1a and Table 1b.

5. The method of claim 4 wherein the reference level of expression is a level from a sample from the same individual wherein the sample is taken at a different time with regard to administration of the asthma treatment.

6. The method of of claim 4 wherein the sample comprises blood cells.

7. The method of claim 6 wherein the blood cells are peripheral blood mononuclear cells (PBMCs).

8. A method for selecting a treatment for asthma, comprising the steps of:
(a) detecting a level of expression of at least one marker in a sample derived from a patient;
(b) comparing the level of expression of the at least one marker to a reference level of expression of the at least one marker;
(c) diagnosing the patient as having asthma; and
(d) selecting a treatment for the patient;
wherein the at least one marker is selected from the group consisting of the markers indicated in Table 1a and Table 1b.

9. A method for identifying an agent capable of modulating expression of at least one marker differentially expressed in asthma, comprising the steps of:
(a) exposing one or more cells to the at least one agent;
(b) determining the level of expression of the at least one marker in the exposed cells;
(c) comparing the level of expression of the at least one marker to a reference level of expression of the at least one marker; and
(d) identifying the agent as capable of modulating the level of expression of the at least one marker based upon the comparison performed in step (c);
wherein said reference level of expression is the level of expression of the at least one marker in a cell not exposed to the agent; and
wherein a change in the level of expression of the at least one marker compared to the reference level of expression of the at least one marker is indicative of the agent's capability of modulating the level of expression of the at least one marker; and
wherein the at least one marker is selected from the group consisting of the markers indicated in Table 1a and Table 1b.

10. The method of claim 9 wherein the reference expression level is selected from a numerical threshold, a level indicative of an asthma state, a level in the same patient at a different time point, a level in the same patient before a treatment regime, or a level in the same patient during a treatment regimen.

11. A method for identifying an agent capable of modulating expression of at least one marker differentially expressed in asthma, comprising the steps of:
(a) administering an agent to a human or non-human mammal;
(b) determining the level of expression of the at least one marker from the treated human or treated non-human mammal;
(c) comparing the level of expression of the at least one marker with a reference level of expression of the at least one marker; and
(d) identifying the agent as capable of modulating the level of expression of the at least one marker in the human or non-human mammal based upon the comparison performed in step (c);
wherein the reference level of expression is the level of expression of the at least one marker in an untreated human or untreated non-human mammal; and
wherein a change in the level of expression of the at least one marker compared to the reference level of expression of the at least one marker is indicative that the agent is capable of modulating the level of expression of the at least one marker; and
wherein the at least one marker is selected from the group consisting of the markers indicated in Table 1a and Table 1b.

12. The method of claim 11 wherein the reference expression level is selected from a numerical threshold, a level indicative of an asthma state, a level in the same patient at a different time point, a level in the same patient before a treatment regime, or a level in the same patient during a treatment regimen.

13. A method for treating an inflammatory disease in a patient, the method comprising the step of modulating the level or activity of at least one marker selected from the group consisting of the markers indicated in Table 2.

14. The method of claim 13 wherein the level or activity is modulated by providing to the patient an isolated nucleic acid complementary to a nucleic acid marker from Table 2.

15. The method of claim 13 wherein the level or activity is modulated by providing to the patient an SiRNA.

16. The method of claim 13 wherein the level or activity is modulated by providing to the patient an isolated antibody to a polypeptide from Table 2.

17. The method of claim 13 wherein the level or activity is modulated by providing to the patient an isolated nucleic acid comprising a nucleic acid from Table 2.

18. The method of claim 13 wherein the level or activity is modulated by providing to the patient an isolated polypeptide from Table 2.

19. An isolated antibody that specifically binds to a polypeptide comprising an amino acid sequence that is at least 95% identical to an amino acid sequence selected from the group consisting of the polypeptide indicated in Table 2.

20. A method of detecting exposure to IL-13 or an IL-13 antagonist comprising the steps of:
(a) detecting a level of expression of at least one marker in one or more cells; and
(b) comparing the level of expression of the at least one marker to a reference level of expression of the at least one marker;
wherein the comparison performed in step (b) is indicative of exposure to IL-13 or an IL-13 antagonist; and
wherein the at least one marker is selected from the group consisting of the markers indicated in Table 7.

21. The method of claim 20 wherein the cells comprise blood cells.

22. The method of claim 21 wherein the blood cells are peripheral blood mononuclear cells (PBMCs).

23. The method according to claim 20 comprising the additional step of providing a diagnosis, prognosis, or assessment of a patient's IL-13-mediated disease based upon the comparison performed in step (b).

24. The method according to claim 20 wherein the one or more cells are derived from a patient during the course of treatment for an IL-13-mediated disease; and wherein the difference between the detected level of expression of the at least one marker and the reference level of expression of the at least one marker is indicative of the effectiveness of the treatment of the IL-13-mediated disease.

25. The method according to claim 20 comprising the step, preceding step (a), of exposing the one or more cells to an agent; and further comprising the step of identifying or evaluating the agent as capable of modulating the level of expression of the at least one marker based upon the comparison performed in step (b);
wherein said reference level of expression is the level of expression of the at least one marker in a cell not exposed to the agent; and
wherein a change in the level of expression of the at least one marker compared to the reference level of expression of the at least one marker is indicative of the agent's capability of modulating the level of expression of the at least one marker.

26. The method according to claim 20 comprising the steps, preceding step (a), of administering an agent to a human or non-human mammal; and deriving a sample comprising one or more cells from the human or non-human mammal; and further comprising the additional step of identifying or evaluating the agent as capable of modulating the level of expression of the at least one marker in the human or non-human mammal based upon the comparison performed in step (b);
wherein the reference level of expression of the at least one marker is the level of expression of the at least one marker in an untreated human or untreated non-human mammal; and
wherein a change in the level of expression of the at least one marker compared to the reference level of expression of the at least one marker is indicative of the agent's capability of modulating the level of expression of the at least one marker.

27. An isolated nucleic acid comprising a nucleic acid sequence selected from the nucleic acid sequences indicated in Table 8.

28. A method for selecting a treatment for an asthma patient comprising:
(a) generating a sample expression profile from a sample derived from the asthma patient;
(b) comparing the sample expression profile to at least one reference expression profile, wherein the at least one reference expression profile represents a favorable clinical outcome in response to a treatment;
(c) selecting a treatment;
wherein the treatment is one that exhibits a reference expression profile that is different from the sample expression profile; and wherein the sample expression profile and the at least one reference expression profile comprise an expression profile of a marker indicated in Table 1a or Table 1b.

29. The method of claim 28 wherein the sample derived from the asthma patient comprises blood cells.

30. The method of claim 28 wherein the blood cells are peripheral blood mononuclear cells (PBMCs).

31. A method of detecting exposure to IL-13, an IL-13 antagonist, or an IL-13 agonist comprising the steps of:
(a) detecting a level of expression of at least one marker in one or more cells; and
(b) comparing the level of expression of the at least one marker to a reference level of expression of the at least one marker;
wherein a difference in the level of expression of the at least one marker and the reference level of expression is indicative of exposure to IL-13, an IL-13 antagonist, or an IL-13 agonist; and
wherein the at least one marker is selected from the group consisting of the markers indicated in Table 7.

32. The method of claim 30 wherein the one or more cells comprise blood cells.

33. The method of claim 30 wherein the blood cells are peripheral blood mononuclear cells (PBMCs).

34. The method according to claim 30 comprising the additional step of providing a diagnosis, prognosis, or assessment of a patient's IL-13-mediated disease based upon the difference in the level of expression of the at least one marker and the reference level of expression.

35. The method according to claim 30 wherein the one or more cells are derived from a patient during the course of treatment for an IL-13-mediated disease; and wherein the difference between the detected level of expression of the at least one marker and the reference level of expression of the at least one marker is indicative of the effectiveness of the treatment of the IL-13-mediated disease.

36. The method according to claim 30 comprising the step, preceding step (a), of exposing the one or more cells to an agent; and further comprising the step of identifying or evaluating the agent as capable of modulating the level of expression of the at least one marker based upon the comparison performed in step (b);
wherein said reference level of expression is the level of expression of the at least one marker in a cell not exposed to the agent; and
wherein a change in the level of expression of the at least one marker compared to the reference level of expression of the at least one marker is indicative of the agent's capability of modulating the level of expression of the at least one marker.

37. The method according to claim 30 comprising the steps, preceding step (a), of administering an agent to a human or non-human mammal; and deriving a sample comprising one or more cells from the human or non-human mammal; and further comprising the additional step of identifying or evaluating the agent as capable of modulating the level of expression of the at least one marker in the human or non-human mammal based upon the comparison performed in step (b);
wherein the reference level of expression of the at least one marker is the level of expression of the at least one marker in an untreated human or untreated non-human mammal; and
wherein a change in the level of expression of the at least one marker compared to the reference level of expression of the at least one marker is indicative of the agent's capability of modulating the level of expression of the at least one marker.


SEQUENCE LISTING (1862 sequences)

SEQ ID NO: 1 - DNA, 25 bp, Artificial
  Probe
tctggcatcagtttgctacagtgag

SEQ ID NO: 2 - DNA, 25 bp, Artificial
  Probe
gaaatgcatgtctcaagctgcaagg

SEQ ID NO: 3 - DNA, 25 bp, Artificial
  Probe
aagctgcaaggcaaactccattcct

SEQ ID NO: 4 - DNA, 25 bp, Artificial
  Probe
aactccattcctcatattaaactat

SEQ ID NO: 5 - DNA, 25 bp, Artificial
  Probe
acttctcatgacgtcaccattttta

SEQ ID NO: 6 - DNA, 25 bp, Artificial
  Probe
tgacgtcaccatttttaactgacag

SEQ ID NO: 7 - DNA, 25 bp, Artificial
  Probe
agacagcaaacttgtgtctgtctct

SEQ ID NO: 8 - DNA, 25 bp, Artificial
  Probe
tttaccacctatgactgtacttgtc

SEQ ID NO: 9 - DNA, 25 bp, Artificial
  Probe
agcagtgattttaaaacctcaagtt

SEQ ID NO: 10 - DNA, 25 bp, Artificial
  Probe
gttgctgtgtaattattgtcttgta

SEQ ID NO: 11 - DNA, 25 bp, Artificial
  Probe
aattattgtcttgtatgcatttgag

SEQ ID NO: 12 - DNA, 25 bp, Artificial
  Probe
gaacagataagtttgcctgcatgct

SEQ ID NO: 13 - DNA, 25 bp, Artificial
  Probe
catgctggacatgcctcagaaccat

SEQ ID NO: 14 - DNA, 25 bp, Artificial
  Probe
tcagaaccatgaatagcccgtacta

SEQ ID NO: 15 - DNA, 25 bp, Artificial
  Probe
tagcccgtactagatcttgggaaca

SEQ ID NO: 16 - DNA, 25 bp, Artificial
  Probe
ggaacatggatcttagagtcacttt

SEQ ID NO: 17 - DNA, 25 bp, Artificial
  Probe
cggggcttgttaaaggacgcgtatg

SEQ ID NO: 18 - DNA, 25 bp, Artificial
  Probe
gtatgtagggcccgtacctactggc

SEQ ID NO: 19 - DNA, 25 bp, Artificial
  Probe
acctactggcagttgggttcaggga

SEQ ID NO: 20 - DNA, 25 bp, Artificial
  Probe
aaatgggattgacttggccttcagg

SEQ ID NO: 21 - DNA, 25 bp, Artificial
  Probe
cttcaggctcctttggtcataattt

SEQ ID NO: 22 - DNA, 25 bp, Artificial
  Probe
aagagcatttatcgtttgtcccttg

SEQ ID NO: 23 - DNA, 25 bp, Artificial
  Probe
gaatagcctgaacctgggaatcgga

SEQ ID NO: 24 - DNA, 25 bp, Artificial
  Probe
cagcctggcaatagaccgagctccg

SEQ ID NO: 25 - DNA, 25 bp, Artificial
  Probe
atggcttcggacaaaatatctctga

SEQ ID NO: 26 - DNA, 25 bp, Artificial
  Probe
aaatatctctgagttctgtgtattt

SEQ ID NO: 27 - DNA, 25 bp, Artificial
  Probe
ttcagtcaaaactttaaacctgtag

SEQ ID NO: 28 - DNA, 25 bp, Artificial
  Probe
aaacctgtagaatcaatttaagtgt

SEQ ID NO: 29 - DNA, 25 bp, Artificial
  Probe
taatttgtttccagcatgaggtatc

SEQ ID NO: 30 - DNA, 25 bp, Artificial
  Probe
tgtttccagcatgaggtatctaagg

SEQ ID NO: 31 - DNA, 25 bp, Artificial
  Probe
agaccagaggtctagattaatactc

SEQ ID NO: 32 - DNA, 25 bp, Artificial
  Probe
ttactctcttccacatgttactgga

SEQ ID NO: 33 - DNA, 25 bp, Artificial
  Probe
tgatgacaatcagttatacagttat

SEQ ID NO: 34 - DNA, 25 bp, Artificial
  Probe
cttttatctcagaaccccatgggtt

SEQ ID NO: 35 - DNA, 25 bp, Artificial
  Probe
tcaaattgttgtcctgtctgtctat

SEQ ID NO: 36 - DNA, 25 bp, Artificial
  Probe
gagctttgattactgactccggttc

SEQ ID NO: 37 - DNA, 25 bp, Artificial
  Probe
ccctgacttaccactaatttactag

SEQ ID NO: 38 - DNA, 25 bp, Artificial
  Probe
tcatgagtaacctctcacagctacc

SEQ ID NO: 39 - DNA, 25 bp, Artificial
  Probe
ccttcttttatctgcactgtgtgaa

SEQ ID NO: 40 - DNA, 25 bp, Artificial
  Probe
gcaagtgtcctaagctatgtcatcc

SEQ ID NO: 41 - DNA, 25 bp, Artificial
  Probe
gtcatccaaagattgtcctttccat

SEQ ID NO: 42 - DNA, 25 bp, Artificial
  Probe
ttccattctcaaatcctgtgactgg

SEQ ID NO: 43 - DNA, 25 bp, Artificial
  Probe
gactgggatcactcaacagcactgt

SEQ ID NO: 44 - DNA, 25 bp, Artificial
  Probe
aagccagtgctctaagacctcagct

SEQ ID NO: 45 - DNA, 25 bp, Artificial
  Probe
gctgccactgaggagttcatcaagc

SEQ ID NO: 46 - DNA, 25 bp, Artificial
  Probe
agcagcacagtcactctacattgcc

SEQ ID NO: 47 - DNA, 25 bp, Artificial
  Probe
ccaaccatgcctactgagtatccac

SEQ ID NO: 48 - DNA, 25 bp, Artificial
  Probe
tactgagtatccactccataccaca

SEQ ID NO: 49 - DNA, 25 bp, Artificial
  Probe
tgcacccacttttgcttgtagtcat

SEQ ID NO: 50 - DNA, 25 bp, Artificial
  Probe
ggccagggccaaatagctatgcaga

SEQ ID NO: 51 - DNA, 25 bp, Artificial
  Probe
agagcagagatgccttcacctggca

SEQ ID NO: 52 - DNA, 25 bp, Artificial
  Probe
cattgctgcacctgggaccatagga

SEQ ID NO: 53 - DNA, 25 bp, Artificial
  Probe
ggaggatagggagcccctcatgact

SEQ ID NO: 54 - DNA, 25 bp, Artificial
  Probe
tcccacaattttcccatgatgaggt

SEQ ID NO: 55 - DNA, 25 bp, Artificial
  Probe
ctgcactcaatgcctggcgagggca

SEQ ID NO: 56 - DNA, 25 bp, Artificial
  Probe
gtgagcatttgttcctgactctcaa

SEQ ID NO: 57 - DNA, 25 bp, Artificial
  Probe
tttggagttctcttacgtttcctgg

SEQ ID NO: 58 - DNA, 25 bp, Artificial
  Probe
ggctggtctcagtttggttactcaa

SEQ ID NO: 59 - DNA, 25 bp, Artificial
  Probe
gcaccagccatatcttttgctttgg

SEQ ID NO: 60 - DNA, 25 bp, Artificial
  Probe
tcacatgatgatacctgcttttctc

SEQ ID NO: 61 - DNA, 25 bp, Artificial
  Probe
catccaacgccctggtttgtaaata

SEQ ID NO: 62 - DNA, 25 bp, Artificial
  Probe
tttggcactggtctggggacattcc

SEQ ID NO: 63 - DNA, 25 bp, Artificial
  Probe
tttcccccttcacagatggtggtgg

SEQ ID NO: 64 - DNA, 25 bp, Artificial
  Probe
ggactctgatgttactcttgagctt

SEQ ID NO: 65 - DNA, 25 bp, Artificial
  Probe
gaaaaccgcaggcttgttgtgttaa

SEQ ID NO: 66 - DNA, 25 bp, Artificial
  Probe
gaaacacaccttcaaacttcaactt

SEQ ID NO: 67 - DNA, 25 bp, Artificial
  Probe
gaaagtctctgtactcgttcatcaa

SEQ ID NO: 68 - DNA, 25 bp, Artificial
  Probe
caagggtgacacatctggagactac

SEQ ID NO: 69 - DNA, 25 bp, Artificial
  Probe
aaagtactgcttgttctctgtggag

SEQ ID NO: 70 - DNA, 25 bp, Artificial
  Probe
ggacaggaggattctcaacactttg

SEQ ID NO: 71 - DNA, 25 bp, Artificial
  Probe
tctacactgctattatcattatctc

SEQ ID NO: 72 - DNA, 25 bp, Artificial
  Probe
ggtgaaatttctaactgttctctgt

SEQ ID NO: 73 - DNA, 25 bp, Artificial
  Probe
tgttctctgttcccggaaccgaaat

SEQ ID NO: 74 - DNA, 25 bp, Artificial
  Probe
ggaaccgaaatcacctgttgcatgt

SEQ ID NO: 75 - DNA, 25 bp, Artificial
  Probe
tgaagatgctctgctggctatagta

SEQ ID NO: 76 - DNA, 25 bp, Artificial
  Probe
ggcaccgatgataacaccctcatca

SEQ ID NO: 77 - DNA, 25 bp, Artificial
  Probe
atatccgggcacacttcaagagact

SEQ ID NO: 78 - DNA, 25 bp, Artificial
  Probe
caagcctattgactaccttaaccca

SEQ ID NO: 79 - DNA, 25 bp, Artificial
  Probe
gttatccttctaaagacttgttctt

SEQ ID NO: 80 - DNA, 25 bp, Artificial
  Probe
ctctacaaattccctcttggtgtaa

SEQ ID NO: 81 - DNA, 25 bp, Artificial
  Probe
taagcacacttacataagcccccat

SEQ ID NO: 82 - DNA, 25 bp, Artificial
  Probe
agcccccatacatagagtgggactc

SEQ ID NO: 83 - DNA, 25 bp, Artificial
  Probe
gtgggactcttggaatcaggagaca

SEQ ID NO: 84 - DNA, 25 bp, Artificial
  Probe
aggagacaaagctaccacatgtgga

SEQ ID NO: 85 - DNA, 25 bp, Artificial
  Probe
aaaggtactatgtgtccatgtcatt

SEQ ID NO: 86 - DNA, 25 bp, Artificial
  Probe
aacacagcagttgcaataaccttgg

SEQ ID NO: 87 - DNA, 25 bp, Artificial
  Probe
ataaccttggcttgtttcggacttg

SEQ ID NO: 88 - DNA, 25 bp, Artificial
  Probe
ggacttgctcgggagggtaatcaca

SEQ ID NO: 89 - DNA, 25 bp, Artificial
  Probe
ttgaatgtttccatgtacctcactt

SEQ ID NO: 90 - DNA, 25 bp, Artificial
  Probe
ccatgtacctcactttatttcagtt

SEQ ID NO: 91 - DNA, 25 bp, Artificial
  Probe
tttggtacatctaagttttcactta

SEQ ID NO: 92 - DNA, 25 bp, Artificial
  Probe
ggatgttgacgccaatgttcagttt

SEQ ID NO: 93 - DNA, 25 bp, Artificial
  Probe
tgttcagtttgggtacgttggtgta

SEQ ID NO: 94 - DNA, 25 bp, Artificial
  Probe
ggtacgttggtgtattgcaagggga

SEQ ID NO: 95 - DNA, 25 bp, Artificial
  Probe
ggttattagggcccattagaaacag

SEQ ID NO: 96 - DNA, 25 bp, Artificial
  Probe
aagctctaaaaaaccatctcatgga

SEQ ID NO: 97 - DNA, 25 bp, Artificial
  Probe
atcatgttctagaaatacctgcaac

SEQ ID NO: 98 - DNA, 25 bp, Artificial
  Probe
atacctgcaacatgacagtctaatc

SEQ ID NO: 99 - DNA, 25 bp, Artificial
  Probe
atgtgtataatttcctggtaaggct

SEQ ID NO: 100 - DNA, 25 bp, Artificial
  Probe
ctaccgctgggtctgcgagacagag

SEQ ID NO: 101 - DNA, 25 bp, Artificial
  Probe
gggtctgcgagacagagctggacaa

SEQ ID NO: 102 - DNA, 25 bp, Artificial
  Probe
tgccgcaggggtccgggattgggaa

SEQ ID NO: 103 - DNA, 25 bp, Artificial
  Probe
tcttctgctttctcgggaattttca

SEQ ID NO: 104 - DNA, 25 bp, Artificial
  Probe
ctcgggaattttcatctaggatttt

SEQ ID NO: 105 - DNA, 25 bp, Artificial
  Probe
gatagggtgatgttccgaaggtgag

SEQ ID NO: 106 - DNA, 25 bp, Artificial
  Probe
ggtgaggagcttgaaacccgtggcg

SEQ ID NO: 107 - DNA, 25 bp, Artificial
  Probe
aggagcagaaatttgtccagcacca

SEQ ID NO: 108 - DNA, 25 bp, Artificial
  Probe
gaaatttgtccagcaccacataggc

SEQ ID NO: 109 - DNA, 25 bp, Artificial
  Probe
ccacataggccctgtgaacacctgg

SEQ ID NO: 110 - DNA, 25 bp, Artificial
  Probe
gagcagccggacgactggtacggcc

SEQ ID NO: 111 - DNA, 25 bp, Artificial
  Probe
gttccccatgtttatgaaagtcctg

SEQ ID NO: 112 - DNA, 25 bp, Artificial
  Probe
aaatattcatgcatgcaattttgac

SEQ ID NO: 113 - DNA, 25 bp, Artificial
  Probe
tgtatatttatggtgggaggtggtt

SEQ ID NO: 114 - DNA, 25 bp, Artificial
  Probe
aatttttgtacagtctgtgggcatt

SEQ ID NO: 115 - DNA, 25 bp, Artificial
  Probe
gtctgtgggcatttacacattttta

SEQ ID NO: 116 - DNA, 25 bp, Artificial
  Probe
aagttacttctagttatgatttgtg

SEQ ID NO: 117 - DNA, 25 bp, Artificial
  Probe
gatttgtgaattccctaagaccttg

SEQ ID NO: 118 - DNA, 25 bp, Artificial
  Probe
aatgatactgcatctttatattttt

SEQ ID NO: 119 - DNA, 25 bp, Artificial
  Probe
aaattgtattgctgctcaagaatgg

SEQ ID NO: 120 - DNA, 25 bp, Artificial
  Probe
agaatggtaccctcttgtcaaaaag

SEQ ID NO: 121 - DNA, 25 bp, Artificial
  Probe
cattcataattgtacattcagcatt

SEQ ID NO: 122 - DNA, 25 bp, Artificial
  Probe
ataccttcagtcaactttaccaaga

SEQ ID NO: 123 - DNA, 25 bp, Artificial
  Probe
gtcctggatttccaagatccgcgtc

SEQ ID NO: 124 - DNA, 25 bp, Artificial
  Probe
actcctccaccgctgagagttgaat

SEQ ID NO: 125 - DNA, 25 bp, Artificial
  Probe
atagcttttcttctgcaatgggagt

SEQ ID NO: 126 - DNA, 25 bp, Artificial
  Probe
ggagtgatgcgtttgattctgccca

SEQ ID NO: 127 - DNA, 25 bp, Artificial
  Probe
cagacagagcccacttagcttgtcc

SEQ ID NO: 128 - DNA, 25 bp, Artificial
  Probe
tggatctcaatgccaatcctccatt

SEQ ID NO: 129 - DNA, 25 bp, Artificial
  Probe
cattcttcctctccagatatttttg

SEQ ID NO: 130 - DNA, 25 bp, Artificial
  Probe
agtgacaaacattctctcatcctac

SEQ ID NO: 131 - DNA, 25 bp, Artificial
  Probe
tagcctacctagatttctcatgacg

SEQ ID NO: 132 - DNA, 25 bp, Artificial
  Probe
gagttaatgcatgtccgtggttggg

SEQ ID NO: 133 - DNA, 25 bp, Artificial
  Probe
cagagcccgagaggaagtttggcgt

SEQ ID NO: 134 - DNA, 25 bp, Artificial
  Probe
agaggaagtttggcgtggtggtggt

SEQ ID NO: 135 - DNA, 25 bp, Artificial
  Probe
ccggctccgtgcggatgagggactt

SEQ ID NO: 136 - DNA, 25 bp, Artificial
  Probe
ggatgagggacttgcggaatccaca

SEQ ID NO: 137 - DNA, 25 bp, Artificial
  Probe
ctgaacctgattggcttcgtgtcga

SEQ ID NO: 138 - DNA, 25 bp, Artificial
  Probe
ggcttcgtgtcgagaagggagctcg

SEQ ID NO: 139 - DNA, 25 bp, Artificial
  Probe
ggagctcgggagcattgatggagtc

SEQ ID NO: 140 - DNA, 25 bp, Artificial
  Probe
gcattgatggagtccagcagatttc

SEQ ID NO: 141 - DNA, 25 bp, Artificial
  Probe
gatttctttggaggatgctctttcc

SEQ ID NO: 142 - DNA, 25 bp, Artificial
  Probe
gatgctctttccagccaagaggtgg

SEQ ID NO: 143 - DNA, 25 bp, Artificial
  Probe
atatctgcagtgagagctccagcca

SEQ ID NO: 144 - DNA, 25 bp, Artificial
  Probe
caaaggacaggcggagaccggccgt

SEQ ID NO: 145 - DNA, 25 bp, Artificial
  Probe
ccgtcatacgcgagcctcatgaaag

SEQ ID NO: 146 - DNA, 25 bp, Artificial
  Probe
aagatccttgcactgctggatgctc

SEQ ID NO: 147 - DNA, 25 bp, Artificial
  Probe
tgctggatgctctgagtacggtgca

SEQ ID NO: 148 - DNA, 25 bp, Artificial
  Probe
gaagctcttcagaattcaggggcag

SEQ ID NO: 149 - DNA, 25 bp, Artificial
  Probe
gggccaattgcagtgagcctttgga

SEQ ID NO: 150 - DNA, 25 bp, Artificial
  Probe
tgtccctggatctgcggaggtagac

SEQ ID NO: 151 - DNA, 25 bp, Artificial
  Probe
gaatgcctgtgaatgacacgtcagt

SEQ ID NO: 152 - DNA, 25 bp, Artificial
  Probe
agatgtctctactcaaactgtgcct

SEQ ID NO: 153 - DNA, 25 bp, Artificial
  Probe
ctgggactgggttcattctcatgac

SEQ ID NO: 154 - DNA, 25 bp, Artificial
  Probe
attctcatgacttggggctgtcgag

SEQ ID NO: 155 - DNA, 25 bp, Artificial
  Probe
atatattgtgcatcaactctgttgg

SEQ ID NO: 156 - DNA, 25 bp, Artificial
  Probe
gtggacgatttgttctagcaccttt

SEQ ID NO: 157 - DNA, 25 bp, Artificial
  Probe
aatggccatcagaatcactatcctc

SEQ ID NO: 158 - DNA, 25 bp, Artificial
  Probe
tcctcctgttccatttggttatcca

SEQ ID NO: 159 - DNA, 25 bp, Artificial
  Probe
aaataaaccatatcgcccaattcca

SEQ ID NO: 160 - DNA, 25 bp, Artificial
  Probe
aattccagtgacatgggtacctcct

SEQ ID NO: 161 - DNA, 25 bp, Artificial
  Probe
tcctcctggaatgcattgtgaccgg

SEQ ID NO: 162 - DNA, 25 bp, Artificial
  Probe
gaccggaatcactggattaatcctc

SEQ ID NO: 163 - DNA, 25 bp, Artificial
  Probe
ttaatcctcacatgttagcacctca

SEQ ID NO: 164 - DNA, 25 bp, Artificial
  Probe
gcacctcactaacttcgtttttgat

SEQ ID NO: 165 - DNA, 25 bp, Artificial
  Probe
tgggccaaaccatcaaacttatttt

SEQ ID NO: 166 - DNA, 25 bp, Artificial
  Probe
gagattattattccttgatgtttgc

SEQ ID NO: 167 - DNA, 25 bp, Artificial
  Probe
tgatgtttgctttgtattggctaca

SEQ ID NO: 168 - DNA, 25 bp, Artificial
  Probe
atgtgatgtcgatgtctctgtcttt

SEQ ID NO: 169 - DNA, 25 bp, Artificial
  Probe
gagaattgaccatttattgttgtga

SEQ ID NO: 170 - DNA, 25 bp, Artificial
  Probe
tgtaatgtgacttatttaacgcctt

SEQ ID NO: 171 - DNA, 25 bp, Artificial
  Probe
ttcctgtctgcacaattagctattc

SEQ ID NO: 172 - DNA, 25 bp, Artificial
  Probe
tagctattcagagcaagagggcctg

SEQ ID NO: 173 - DNA, 25 bp, Artificial
  Probe
gcctgattttatagaagccccttga

SEQ ID NO: 174 - DNA, 25 bp, Artificial
  Probe
gaagccccttgaaaagaggtccaga

SEQ ID NO: 175 - DNA, 25 bp, Artificial
  Probe
aattatgtgatctgtgtgttgtggg

SEQ ID NO: 176 - DNA, 25 bp, Artificial
  Probe
tacggagctgtagtgccattagaaa

SEQ ID NO: 177 - DNA, 25 bp, Artificial
  Probe
attctactcataggctttaccaagt

SEQ ID NO: 178 - DNA, 25 bp, Artificial
  Probe
caagatcagttggcagtatctgctc

SEQ ID NO: 179 - DNA, 25 bp, Artificial
  Probe
ggcagtatctgctcaagaacattct

SEQ ID NO: 180 - DNA, 25 bp, Artificial
  Probe
tttctttctgtccaaacggaataag

SEQ ID NO: 181 - DNA, 25 bp, Artificial
  Probe
gtggacatgctttgtgatactttgt

SEQ ID NO: 182 - DNA, 25 bp, Artificial
  Probe
cagcttttgcttagtgatcaggagt

SEQ ID NO: 183 - DNA, 25 bp, Artificial
  Probe
gaataagctaaatcatctcctcact

SEQ ID NO: 184 - DNA, 25 bp, Artificial
  Probe
catctcctcactgatattcttgctg

SEQ ID NO: 185 - DNA, 25 bp, Artificial
  Probe
gattaaggctgttagtcttgaagat

SEQ ID NO: 186 - DNA, 25 bp, Artificial
  Probe
gaatctttattacgtgtcctctttt

SEQ ID NO: 187 - DNA, 25 bp, Artificial
  Probe
ttacgtgtcctcttttatttattag

SEQ ID NO: 188 - DNA, 25 bp, Artificial
  Probe
aggctgaagtaaccttattcctatt

SEQ ID NO: 189 - DNA, 25 bp, Artificial
  Probe
accttattcctattgtttagtagct

SEQ ID NO: 190 - DNA, 25 bp, Artificial
  Probe
agtagctaatagcatgcttttgata

SEQ ID NO: 191 - DNA, 25 bp, Artificial
  Probe
gcatgcttttgatatgcttatgatc

SEQ ID NO: 192 - DNA, 25 bp, Artificial
  Probe
attgtgatgctgtatcatattttat

SEQ ID NO: 193 - DNA, 25 bp, Artificial
  Probe
tacggtttataagaaaagctcctag

SEQ ID NO: 194 - DNA, 25 bp, Artificial
  Probe
agaaaagctcctaggtataaaatgc

SEQ ID NO: 195 - DNA, 25 bp, Artificial
  Probe
aatgctacatagcaggaacttggtt

SEQ ID NO: 196 - DNA, 25 bp, Artificial
  Probe
gcaggaacttggtttttcaatgtta

SEQ ID NO: 197 - DNA, 25 bp, Artificial
  Probe
atgttattatttcctactgtttttg

SEQ ID NO: 198 - DNA, 25 bp, Artificial
  Probe
tcctactgtttttgacgtaacggca

SEQ ID NO: 199 - DNA, 25 bp, Artificial
  Probe
gaagctgtgcttcgtgctatagatg

SEQ ID NO: 200 - DNA, 25 bp, Artificial
  Probe
atagatggcatcaaccagcggtcct

SEQ ID NO: 201 - DNA, 25 bp, Artificial
  Probe
gaccccaggcccacgatggatgggg

SEQ ID NO: 202 - DNA, 25 bp, Artificial
  Probe
atggatggggacccagacacgccaa

SEQ ID NO: 203 - DNA, 25 bp, Artificial
  Probe
gacacgccaaagcctgtgagcttca

SEQ ID NO: 204 - DNA, 25 bp, Artificial
  Probe
gacgacacagcagtcaccagaggat

SEQ ID NO: 205 - DNA, 25 bp, Artificial
  Probe
ggtgtatggggacagtgaccctcaa

SEQ ID NO: 206 - DNA, 25 bp, Artificial
  Probe
cccaaagggatggccactccctggg

SEQ ID NO: 207 - DNA, 25 bp, Artificial
  Probe
ggctcctttgacatcagttgtgata

SEQ ID NO: 208 - DNA, 25 bp, Artificial
  Probe
gatttgccctgctgggtgatttctt

SEQ ID NO: 209 - DNA, 25 bp, Artificial
  Probe
ttttgcggaatcttgtacccaggac

SEQ ID NO: 210 - DNA, 25 bp, Artificial
  Probe
agcgatctgccatgagagcagtagc

SEQ ID NO: 211 - DNA, 25 bp, Artificial
  Probe
gagtccactgatgagtgaattccag

SEQ ID NO: 212 - DNA, 25 bp, Artificial
  Probe
gaattccagtcacagatcagttcta

SEQ ID NO: 213 - DNA, 25 bp, Artificial
  Probe
gatcagttctaaccctgagctggcg

SEQ ID NO: 214 - DNA, 25 bp, Artificial
  Probe
gagctggcggctatctttgaaagta

SEQ ID NO: 215 - DNA, 25 bp, Artificial
  Probe
tcatcatctactaacttggaatcaa

SEQ ID NO: 216 - DNA, 25 bp, Artificial
  Probe
gacactagttagatgtttgttcacc

SEQ ID NO: 217 - DNA, 25 bp, Artificial
  Probe
gtttgttcaccatggggaccattac

SEQ ID NO: 218 - DNA, 25 bp, Artificial
  Probe
ggggaccattacatatgaccataca

SEQ ID NO: 219 - DNA, 25 bp, Artificial
  Probe
atttccataatccagaggttgtaaa

SEQ ID NO: 220 - DNA, 25 bp, Artificial
  Probe
ggtccagtatctatttaccctgtaa

SEQ ID NO: 221 - DNA, 25 bp, Artificial
  Probe
ggcattgaggccagtcctgacaaaa

SEQ ID NO: 222 - DNA, 25 bp, Artificial
  Probe
cgcctgggacccaattatcttcata

SEQ ID NO: 223 - DNA, 25 bp, Artificial
  Probe
tcatatacctgtgaactgtccctac

SEQ ID NO: 224 - DNA, 25 bp, Artificial
  Probe
ggcccgatgtgcatgcaggacaatc

SEQ ID NO: 225 - DNA, 25 bp, Artificial
  Probe
gctttggtgctccggaacaacagcc

SEQ ID NO: 226 - DNA, 25 bp, Artificial
  Probe
gtgcggagattcaacactgccaatg

SEQ ID NO: 227 - DNA, 25 bp, Artificial
  Probe
taacgttactcaggtgcgggcattc

SEQ ID NO: 228 - DNA, 25 bp, Artificial
  Probe
tcaagaacttcactgaggtccaccc

SEQ ID NO: 229 - DNA, 25 bp, Artificial
  Probe
tgactacgggagccacatccaggct

SEQ ID NO: 230 - DNA, 25 bp, Artificial
  Probe
aagaatgcgattcacacctttgtgc

SEQ ID NO: 231 - DNA, 25 bp, Artificial
  Probe
ggttgaacagatagccttcgaccca

SEQ ID NO: 232 - DNA, 25 bp, Artificial
  Probe
aagaaagccaaggcggacagccccg

SEQ ID NO: 233 - DNA, 25 bp, Artificial
  Probe
cggagccgtgagcagagctactcga

SEQ ID NO: 234 - DNA, 25 bp, Artificial
  Probe
tcccgatcagcgtctcctaagagga

SEQ ID NO: 235 - DNA, 25 bp, Artificial
  Probe
gaaaagtgacagcggctccacatct

SEQ ID NO: 236 - DNA, 25 bp, Artificial
  Probe
cacatctggtgggtccaagtcgcag

SEQ ID NO: 237 - DNA, 25 bp, Artificial
  Probe
agccgctcccggagcaggagtgact

SEQ ID NO: 238 - DNA, 25 bp, Artificial
  Probe
cgctccctacaaaggctctgagatt

SEQ ID NO: 239 - DNA, 25 bp, Artificial
  Probe
tgagattcggggctcccggaagtcc

SEQ ID NO: 240 - DNA, 25 bp, Artificial
  Probe
caagtctcggagccggagttcttcc

SEQ ID NO: 241 - DNA, 25 bp, Artificial
  Probe
ttcccgttctcgaagcaggtcacgg

SEQ ID NO: 242 - DNA, 25 bp, Artificial
  Probe
gaggtcgtatgaacgcacaggccgt

SEQ ID NO: 243 - DNA, 25 bp, Artificial
  Probe
ttgtgtgagctattcaaactcttca

SEQ ID NO: 244 - DNA, 25 bp, Artificial
  Probe
actcttcaacccctgaacagggtat

SEQ ID NO: 245 - DNA, 25 bp, Artificial
  Probe
gaacagggtattaagcttccaaaat

SEQ ID NO: 246 - DNA, 25 bp, Artificial
  Probe
aaacccttataattcatactatcat

SEQ ID NO: 247 - DNA, 25 bp, Artificial
  Probe
gaatttgctttatccatctcatttg

SEQ ID NO: 248 - DNA, 25 bp, Artificial
  Probe
atctcatttgcataacagttcatct

SEQ ID NO: 249 - DNA, 25 bp, Artificial
  Probe
taacagttcatctgtctggtcccat

SEQ ID NO: 250 - DNA, 25 bp, Artificial
  Probe
ggtcccattaggctctaccaaagaa

SEQ ID NO: 251 - DNA, 25 bp, Artificial
  Probe
tgagtggacattattactgtgactc

SEQ ID NO: 252 - DNA, 25 bp, Artificial
  Probe
actgtgactcttgtaagtagccata

SEQ ID NO: 253 - DNA, 25 bp, Artificial
  Probe
aggtatgaaattccacatgtgcaaa

SEQ ID NO: 254 - DNA, 25 bp, Artificial
  Probe
tccatcgctgtcatctcagctggat

SEQ ID NO: 255 - DNA, 25 bp, Artificial
  Probe
ttctctcaggcttgctgccaaaagc

SEQ ID NO: 256 - DNA, 25 bp, Artificial
  Probe
attcgagtgtttcagtgcttcgcag

SEQ ID NO: 257 - DNA, 25 bp, Artificial
  Probe
gtgcttcgcagatgtccttgatgct

SEQ ID NO: 258 - DNA, 25 bp, Artificial
  Probe
gctcatattgttccctaatttgcca

SEQ ID NO: 259 - DNA, 25 bp, Artificial
  Probe
actttcctcttagtcgagccaagtt

SEQ ID NO: 260 - DNA, 25 bp, Artificial
  Probe
gtgtgtttctgatctgatgcaagca

SEQ ID NO: 261 - DNA, 25 bp, Artificial
  Probe
tgggcttctagaaccaggcaacttg

SEQ ID NO: 262 - DNA, 25 bp, Artificial
  Probe
ggaactagactcccaagctggacta

SEQ ID NO: 263 - DNA, 25 bp, Artificial
  Probe
gctggactatggctctactttcagg

SEQ ID NO: 264 - DNA, 25 bp, Artificial
  Probe
gacagagcagaactttcaccttcat

SEQ ID NO: 265 - DNA, 25 bp, Artificial
  Probe
gtgcctaaaggactgccagccaagc

SEQ ID NO: 266 - DNA, 25 bp, Artificial
  Probe
gccaagctcagagtgctcgatctca

SEQ ID NO: 267 - DNA, 25 bp, Artificial
  Probe
gcaacagactgaacagggcgccgca

SEQ ID NO: 268 - DNA, 25 bp, Artificial
  Probe
tgacgagctgcccgaggtggataac

SEQ ID NO: 269 - DNA, 25 bp, Artificial
  Probe
ctgacactggacgggaatcccttcc

SEQ ID NO: 270 - DNA, 25 bp, Artificial
  Probe
acgagggctcaatgaactccggcgt

SEQ ID NO: 271 - DNA, 25 bp, Artificial
  Probe
cccggggctttgcctaagatccaag

SEQ ID NO: 272 - DNA, 25 bp, Artificial
  Probe
gggagtcccgtcaggacgttgagga

SEQ ID NO: 273 - DNA, 25 bp, Artificial
  Probe
tgaggacttttcgaccaattcaacc

SEQ ID NO: 274 - DNA, 25 bp, Artificial
  Probe
ccatccagaatctagcgctgcgcaa

SEQ ID NO: 275 - DNA, 25 bp, Artificial
  Probe
ccctagcgctccgagatgcatgtgg

SEQ ID NO: 276 - DNA, 25 bp, Artificial
  Probe
gccagacgctggggccatagtgagt

SEQ ID NO: 277 - DNA, 25 bp, Artificial
  Probe
cgtcagtcatcctttattgcagtcg

SEQ ID NO: 278 - DNA, 25 bp, Artificial
  Probe
tattgcagtcgggatccttggggtt

SEQ ID NO: 279 - DNA, 25 bp, Artificial
  Probe
ggccattttcgtcgcattattcttc

SEQ ID NO: 280 - DNA, 25 bp, Artificial
  Probe
cagagacagcggcttgcagtttcct

SEQ ID NO: 281 - DNA, 25 bp, Artificial
  Probe
ttagtccaccaaattcaataccggg

SEQ ID NO: 282 - DNA, 25 bp, Artificial
  Probe
taccgggagatgaattcttgcctga

SEQ ID NO: 283 - DNA, 25 bp, Artificial
  Probe
gccattctgagccacactgaaaagg

SEQ ID NO: 284 - DNA, 25 bp, Artificial
  Probe
ataacccagtgagttcagcctttaa

SEQ ID NO: 285 - DNA, 25 bp, Artificial
  Probe
tggagcagaaattcacctctctcac

SEQ ID NO: 286 - DNA, 25 bp, Artificial
  Probe
ggagttcttcttctcctaggattcc

SEQ ID NO: 287 - DNA, 25 bp, Artificial
  Probe
cgtcagtcatcctttattgcagtcg

SEQ ID NO: 288 - DNA, 25 bp, Artificial
  Probe
tattgcagtcgggatccttggggtt

SEQ ID NO: 289 - DNA, 25 bp, Artificial
  Probe
ggccattttcgtcgcattattcttc

SEQ ID NO: 290 - DNA, 25 bp, Artificial
  Probe
cagagacagcggcttgcagtttcct

SEQ ID NO: 291 - DNA, 25 bp, Artificial
  Probe
aattcccatgagtcagctgatttca

SEQ ID NO: 292 - DNA, 25 bp, Artificial
  Probe
aaaggaggccattctgagccacact

SEQ ID NO: 293 - DNA, 25 bp, Artificial
  Probe
ataacccagtgagttcagcctttaa

SEQ ID NO: 294 - DNA, 25 bp, Artificial
  Probe
tggagcagaaattcacctctctcac

SEQ ID NO: 295 - DNA, 25 bp, Artificial
  Probe
tcacctctctcactgactattacag

SEQ ID NO: 296 - DNA, 25 bp, Artificial
  Probe
ggagttcttcttctcctaggattcc

SEQ ID NO: 297 - DNA, 25 bp, Artificial
  Probe
ctaggattcctaagactgctgctga

SEQ ID NO: 298 - DNA, 25 bp, Artificial
  Probe
ctgggattacaggcttgagcccccg

SEQ ID NO: 299 - DNA, 25 bp, Artificial
  Probe
gcgcccagccatcaaaatgcttttt

SEQ ID NO: 300 - DNA, 25 bp, Artificial
  Probe
gctttttatttctgcatatgtttga

SEQ ID NO: 301 - DNA, 25 bp, Artificial
  Probe
tcacaaacttttatactctttctgt

SEQ ID NO: 302 - DNA, 25 bp, Artificial
  Probe
tctgtatatacattttttttcttta

SEQ ID NO: 303 - DNA, 25 bp, Artificial
  Probe
aatagccacatttagaacacttttt

SEQ ID NO: 304 - DNA, 25 bp, Artificial
  Probe
aacactttttgttatcagtcaatat

SEQ ID NO: 305 - DNA, 25 bp, Artificial
  Probe
gatagttagaacctggtcctaagcc

SEQ ID NO: 306 - DNA, 25 bp, Artificial
  Probe
gaacctggtcctaagcctaaaagtg

SEQ ID NO: 307 - DNA, 25 bp, Artificial
  Probe
aagtgggcttgattctgcagtaaat

SEQ ID NO: 308 - DNA, 25 bp, Artificial
  Probe
taaatcttttacaactgcctcgaca

SEQ ID NO: 309 - DNA, 25 bp, Artificial
  Probe
tcacaaacttttatactctttctgt

SEQ ID NO: 310 - DNA, 25 bp, Artificial
  Probe
gaacctggtcctaagcctaaaagtg

SEQ ID NO: 311 - DNA, 25 bp, Artificial
  Probe
aagtgggcttgattctgcagtaaat

SEQ ID NO: 312 - DNA, 25 bp, Artificial
  Probe
taaatcttttacaactgcctcgaca

SEQ ID NO: 313 - DNA, 25 bp, Artificial
  Probe
gcctcgacacacataaaccttttta

SEQ ID NO: 314 - DNA, 25 bp, Artificial
  Probe
aatagacactccccgaagtcttttg

SEQ ID NO: 315 - DNA, 25 bp, Artificial
  Probe
aagtcttttgttcgcatggtcacac

SEQ ID NO: 316 - DNA, 25 bp, Artificial
  Probe
tatggccacagtagtcttgatgacc

SEQ ID NO: 317 - DNA, 25 bp, Artificial
  Probe
tgaccaaagtcctttttttccatct

SEQ ID NO: 318 - DNA, 25 bp, Artificial
  Probe
gaacactcttgctttattccagaat

SEQ ID NO: 319 - DNA, 25 bp, Artificial
  Probe
gtgtatttacgctttgattcatagt

SEQ ID NO: 320 - DNA, 25 bp, Artificial
  Probe
tcacaaacttttatactctttctgt

SEQ ID NO: 321 - DNA, 25 bp, Artificial
  Probe
gaacctggtcctaagcctaaaagtg

SEQ ID NO: 322 - DNA, 25 bp, Artificial
  Probe
aagtgggcttgattctgcagtaaat

SEQ ID NO: 323 - DNA, 25 bp, Artificial
  Probe
taaatcttttacaactgcctcgaca

SEQ ID NO: 324 - DNA, 25 bp, Artificial
  Probe
gcctcgacacacataaaccttttta

SEQ ID NO: 325 - DNA, 25 bp, Artificial
  Probe
aatagacactccccgaagtcttttg

SEQ ID NO: 326 - DNA, 25 bp, Artificial
  Probe
gaagtcttttgttcgcatggtcaca

SEQ ID NO: 327 - DNA, 25 bp, Artificial
  Probe
catggtcacacactgatgcttagat

SEQ ID NO: 328 - DNA, 25 bp, Artificial
  Probe
tatggccacagtagtcttgatgacc

SEQ ID NO: 329 - DNA, 25 bp, Artificial
  Probe
tgaccaaagtcctttttttccatct

SEQ ID NO: 330 - DNA, 25 bp, Artificial
  Probe
gaacactcttgctttattccagaat

SEQ ID NO: 331 - DNA, 25 bp, Artificial
  Probe
tagtctaattgaatcccttaaactc

SEQ ID NO: 332 - DNA, 25 bp, Artificial
  Probe
atgggatgatcgtgtatttattttt

SEQ ID NO: 333 - DNA, 25 bp, Artificial
  Probe
ttttttacttcctcagctgtagaca

SEQ ID NO: 334 - DNA, 25 bp, Artificial
  Probe
acttcctcagctgtagacaggtcct

SEQ ID NO: 335 - DNA, 25 bp, Artificial
  Probe
gacaggtccttttcgatggtacata

SEQ ID NO: 336 - DNA, 25 bp, Artificial
  Probe
tggtacatatttctttgcctttata

SEQ ID NO: 337 - DNA, 25 bp, Artificial
  Probe
tataatcttttatacagtgtcttac

SEQ ID NO: 338 - DNA, 25 bp, Artificial
  Probe
gtgatgtggcaaatctctattagga

SEQ ID NO: 339 - DNA, 25 bp, Artificial
  Probe
atattctgtaatcttcagacctaga

SEQ ID NO: 340 - DNA, 25 bp, Artificial
  Probe
aggtttgtgactttcctaaatcaat

SEQ ID NO: 341 - DNA, 25 bp, Artificial
  Probe
tacgtgcaatacttcaatacttcat

SEQ ID NO: 342 - DNA, 25 bp, Artificial
  Probe
tccatttctcatgttttccattgtt

SEQ ID NO: 343 - DNA, 25 bp, Artificial
  Probe
caagaagcctttcctgtagccttct

SEQ ID NO: 344 - DNA, 25 bp, Artificial
  Probe
gtccacggtctgttcttgaagcagt

SEQ ID NO: 345 - DNA, 25 bp, Artificial
  Probe
tgaagcagtagcctaacacactcca

SEQ ID NO: 346 - DNA, 25 bp, Artificial
  Probe
aagatatggacacacgggagccgct

SEQ ID NO: 347 - DNA, 25 bp, Artificial
  Probe
gttttagccattgttggctttccct

SEQ ID NO: 348 - DNA, 25 bp, Artificial
  Probe
tggctttcccttatcaaacttgggc

SEQ ID NO: 349 - DNA, 25 bp, Artificial
  Probe
ctgagttatatgttcactgtccccc

SEQ ID NO: 350 - DNA, 25 bp, Artificial
  Probe
gttcactgtccccctaatattaggg

SEQ ID NO: 351 - DNA, 25 bp, Artificial
  Probe
gaacccccatgatgtaagtttacct

SEQ ID NO: 352 - DNA, 25 bp, Artificial
  Probe
aaacctgcacttatacccatgaact

SEQ ID NO: 353 - DNA, 25 bp, Artificial
  Probe
tgtgctgaacggaccgctatccaga

SEQ ID NO: 354 - DNA, 25 bp, Artificial
  Probe
ctatccagaaggccgtctcagaagg

SEQ ID NO: 355 - DNA, 25 bp, Artificial
  Probe
gcaattgctatcgccagtgacatgc

SEQ ID NO: 356 - DNA, 25 bp, Artificial
  Probe
ggggcctgcaggcaagtcatgagag

SEQ ID NO: 357 - DNA, 25 bp, Artificial
  Probe
gagagagtttggcaccaactggccc

SEQ ID NO: 358 - DNA, 25 bp, Artificial
  Probe
gcccgtgtacatgaccaagccggat

SEQ ID NO: 359 - DNA, 25 bp, Artificial
  Probe
tgtcatgacggtccaggagctgctg

SEQ ID NO: 360 - DNA, 25 bp, Artificial
  Probe
tcctttgggcctgaggacctgcaga

SEQ ID NO: 361 - DNA, 25 bp, Artificial
  Probe
agtgacagccagagaatgcccactg

SEQ ID NO: 362 - DNA, 25 bp, Artificial
  Probe
tgccttgggacttagaacaccgccg

SEQ ID NO: 363 - DNA, 25 bp, Artificial
  Probe
gtccagcctagtctggactgcttcc

SEQ ID NO: 364 - DNA, 25 bp, Artificial
  Probe
attgccaattctttaagtgttttct

SEQ ID NO: 365 - DNA, 25 bp, Artificial
  Probe
ttaagctatctataccttactgcaa

SEQ ID NO: 366 - DNA, 25 bp, Artificial
  Probe
cctacctgactgccacagaactggg

SEQ ID NO: 367 - DNA, 25 bp, Artificial
  Probe
agttcagtgagaatctgctgtcttt

SEQ ID NO: 368 - DNA, 25 bp, Artificial
  Probe
agtgtctaatctatcgtgtcaaccc

SEQ ID NO: 369 - DNA, 25 bp, Artificial
  Probe
atctatcgtgtcaaccccaaatttt

SEQ ID NO: 370 - DNA, 25 bp, Artificial
  Probe
tacgtatgagatcctttagtccacc

SEQ ID NO: 371 - DNA, 25 bp, Artificial
  Probe
ttagtccacccaatggctgacagta

SEQ ID NO: 372 - DNA, 25 bp, Artificial
  Probe
gcatctttaacacaactctttgttc

SEQ ID NO: 373 - DNA, 25 bp, Artificial
  Probe
gttcaaatgtactatggtctctttt

SEQ ID NO: 374 - DNA, 25 bp, Artificial
  Probe
taatttaacccaggcatgcaatgct

SEQ ID NO: 375 - DNA, 25 bp, Artificial
  Probe
gctgtggggatagtgaggcatcgca

SEQ ID NO: 376 - DNA, 25 bp, Artificial
  Probe
ggggatagtgaggcatcgcaatgta

SEQ ID NO: 377 - DNA, 25 bp, Artificial
  Probe
gaggcatcgcaatgtaagactcggg

SEQ ID NO: 378 - DNA, 25 bp, Artificial
  Probe
gcatcgcaatgtaagactcgggatt

SEQ ID NO: 379 - DNA, 25 bp, Artificial
  Probe
gtaagactcgggattagtacacact

SEQ ID NO: 380 - DNA, 25 bp, Artificial
  Probe
gactcgggattagtacacacttgtt

SEQ ID NO: 381 - DNA, 25 bp, Artificial
  Probe
ggattagtacacacttgttgatgat

SEQ ID NO: 382 - DNA, 25 bp, Artificial
  Probe
ggaaatgtttacagatccccaagcc

SEQ ID NO: 383 - DNA, 25 bp, Artificial
  Probe
aaatgtttacagatccccaagcctg

SEQ ID NO: 384 - DNA, 25 bp, Artificial
  Probe
accttcacttaggaacgtaatcgtg

SEQ ID NO: 385 - DNA, 25 bp, Artificial
  Probe
taggaacgtaatcgtgtcccctatc

SEQ ID NO: 386 - DNA, 25 bp, Artificial
  Probe
tcaccaatgccatcaaggatgcact

SEQ ID NO: 387 - DNA, 25 bp, Artificial
  Probe
caaggatgcactcgctgcaacgtag

SEQ ID NO: 388 - DNA, 25 bp, Artificial
  Probe
cacacagcacgggggccaaggatgc

SEQ ID NO: 389 - DNA, 25 bp, Artificial
  Probe
tgcagaggtccacaacacacagatt

SEQ ID NO: 390 - DNA, 25 bp, Artificial
  Probe
cacagatttgagctcagccctggtg

SEQ ID NO: 391 - DNA, 25 bp, Artificial
  Probe
ccctagccctccttatcaaaggaca

SEQ ID NO: 392 - DNA, 25 bp, Artificial
  Probe
aaggacaccattttggcaagctcta

SEQ ID NO: 393 - DNA, 25 bp, Artificial
  Probe
ggcaagctctatcaccaaggagcca

SEQ ID NO: 394 - DNA, 25 bp, Artificial
  Probe
atcctacaagacacagtgaccatac

SEQ ID NO: 395 - DNA, 25 bp, Artificial
  Probe
agtgaccatactaattataccccct

SEQ ID NO: 396 - DNA, 25 bp, Artificial
  Probe
gcaaagccagcttgaaaccttcact

SEQ ID NO: 397 - DNA, 25 bp, Artificial
  Probe
ttcccattaacctttgccagtgtta

SEQ ID NO: 398 - DNA, 25 bp, Artificial
  Probe
tgctactttgagttttgtttcgtat

SEQ ID NO: 399 - DNA, 25 bp, Artificial
  Probe
gtttcgtatcatgtcctatgctaga

SEQ ID NO: 400 - DNA, 25 bp, Artificial
  Probe
aatttgaactacagctggactccgt

SEQ ID NO: 401 - DNA, 25 bp, Artificial
  Probe
gctggactccgtttgtgtgatggtg

SEQ ID NO: 402 - DNA, 25 bp, Artificial
  Probe
gatacatgtcattagttgcaacttc

SEQ ID NO: 403 - DNA, 25 bp, Artificial
  Probe
attgtctattggttattgatcttgc

SEQ ID NO: 404 - DNA, 25 bp, Artificial
  Probe
tgtcccttctatgatcccttaagaa

SEQ ID NO: 405 - DNA, 25 bp, Artificial
  Probe
aagctgcaccaaatcatctgcctgt

SEQ ID NO: 406 - DNA, 25 bp, Artificial
  Probe
gcctgttttttcttgatacttactg

SEQ ID NO: 407 - DNA, 25 bp, Artificial
  Probe
ttttggtttgtttatatctttgttg

SEQ ID NO: 408 - DNA, 25 bp, Artificial
  Probe
caaagggcgaccacttgtctgtttc

SEQ ID NO: 409 - DNA, 25 bp, Artificial
  Probe
ccaagtgtgctttggtcgtcgtgtg

SEQ ID NO: 410 - DNA, 25 bp, Artificial
  Probe
gtgagtatggggcctggaagcagca

SEQ ID NO: 411 - DNA, 25 bp, Artificial
  Probe
gaacatgccctttcaggatggccaa

SEQ ID NO: 412 - DNA, 25 bp, Artificial
  Probe
tggtcaatggccaatcctcttacac

SEQ ID NO: 413 - DNA, 25 bp, Artificial
  Probe
aggagacaacaatgtccctgctacc

SEQ ID NO: 414 - DNA, 25 bp, Artificial
  Probe
tcctcttacacctttgaccatagaa

SEQ ID NO: 415 - DNA, 25 bp, Artificial
  Probe
gataaccagacttcatgttgccaag

SEQ ID NO: 416 - DNA, 25 bp, Artificial
  Probe
tgttgccaaggaatccctgtctcta

SEQ ID NO: 417 - DNA, 25 bp, Artificial
  Probe
gtctctacgtgaacttgggattcca

SEQ ID NO: 418 - DNA, 25 bp, Artificial
  Probe
tctttgtctactggttctactgtga

SEQ ID NO: 419 - DNA, 25 bp, Artificial
  Probe
ggaaaggattcttggacctctacca

SEQ ID NO: 420 - DNA, 25 bp, Artificial
  Probe
ggacctctaccaaaacatatgatac

SEQ ID NO: 421 - DNA, 25 bp, Artificial
  Probe
gtaaatattttcaccacgatcgatt

SEQ ID NO: 422 - DNA, 25 bp, Artificial
  Probe
accacgatcgattagactgggatga

SEQ ID NO: 423 - DNA, 25 bp, Artificial
  Probe
tgaacacagttctgccggcagatat

SEQ ID NO: 424 - DNA, 25 bp, Artificial
  Probe
ttctgccggcagatatgtttcaaga

SEQ ID NO: 425 - DNA, 25 bp, Artificial
  Probe
aatgttggagtatgatccagccaaa

SEQ ID NO: 426 - DNA, 25 bp, Artificial
  Probe
cagccaaaagaattactctcagaga

SEQ ID NO: 427 - DNA, 25 bp, Artificial
  Probe
tgtaattggacagctctctcgaaga

SEQ ID NO: 428 - DNA, 25 bp, Artificial
  Probe
gacagctctctcgaagagatcttac

SEQ ID NO: 429 - DNA, 25 bp, Artificial
  Probe
ggtaatgaacatctttttcagtaat

SEQ ID NO: 430 - DNA, 25 bp, Artificial
  Probe
cagatggtcatctggattctcccac

SEQ ID NO: 431 - DNA, 25 bp, Artificial
  Probe
ttccttccagcaaaccttgaaacgt

SEQ ID NO: 432 - DNA, 25 bp, Artificial
  Probe
gtgagtaacaggaatgtgtctttaa

SEQ ID NO: 433 - DNA, 25 bp, Artificial
  Probe
tagagtggttacatttaatcaggca

SEQ ID NO: 434 - DNA, 25 bp, Artificial
  Probe
gataatttgggttcttgagttgttt

SEQ ID NO: 435 - DNA, 25 bp, Artificial
  Probe
ggagtaatatcccacaactggggta

SEQ ID NO: 436 - DNA, 25 bp, Artificial
  Probe
aactggggtaggaagctcaggactt

SEQ ID NO: 437 - DNA, 25 bp, Artificial
  Probe
ttttctttaaagctagtcatttcaa

SEQ ID NO: 438 - DNA, 25 bp, Artificial
  Probe
aaaactggtaactcactcaagtgaa

SEQ ID NO: 439 - DNA, 25 bp, Artificial
  Probe
gtgaatgaatggtcttgcattttaa

SEQ ID NO: 440 - DNA, 25 bp, Artificial
  Probe
aaagcttatgggaaactcaatttga

SEQ ID NO: 441 - DNA, 25 bp, Artificial
  Probe
ggtgactatgctatttcagttcgta

SEQ ID NO: 442 - DNA, 25 bp, Artificial
  Probe
gtaatggacctgaaagtggcagcaa

SEQ ID NO: 443 - DNA, 25 bp, Artificial
  Probe
ggcagcaagattatggttcagtttc

SEQ ID NO: 444 - DNA, 25 bp, Artificial
  Probe
gttcagtttcctcgtaaccaatgta

SEQ ID NO: 445 - DNA, 25 bp, Artificial
  Probe
tcgtaaccaatgtaaagaccttcca

SEQ ID NO: 446 - DNA, 25 bp, Artificial
  Probe
agtcatcttcgtgggccattcaaag

SEQ ID NO: 447 - DNA, 25 bp, Artificial
  Probe
ggagctgcttatgtctgcacttagc

SEQ ID NO: 448 - DNA, 25 bp, Artificial
  Probe
cacttagcccttgtctactatgatt

SEQ ID NO: 449 - DNA, 25 bp, Artificial
  Probe
gatgtttcctaaagaagtttccaga

SEQ ID NO: 450 - DNA, 25 bp, Artificial
  Probe
gataacttccaaaagagtgctgttt

SEQ ID NO: 451 - DNA, 25 bp, Artificial
  Probe
aatattccttctttgatgttgacat

SEQ ID NO: 452 - DNA, 25 bp, Artificial
  Probe
ggttccatcaatggtgagcaccagc

SEQ ID NO: 453 - DNA, 25 bp, Artificial
  Probe
tggtgagcaccagcctgaatgcaga

SEQ ID NO: 454 - DNA, 25 bp, Artificial
  Probe
gcaccagcctgaatgcagaagcgct

SEQ ID NO: 455 - DNA, 25 bp, Artificial
  Probe
gaatgcagaagcgctccagtatctc

SEQ ID NO: 456 - DNA, 25 bp, Artificial
  Probe
ctccagtatctccaagggtaccttc

SEQ ID NO: 457 - DNA, 25 bp, Artificial
  Probe
tctccaagggtaccttcaggcagcc

SEQ ID NO: 458 - DNA, 25 bp, Artificial
  Probe
agtgtgacactgctttaaactgcat

SEQ ID NO: 459 - DNA, 25 bp, Artificial
  Probe
gacactgctttaaactgcatttttc

SEQ ID NO: 460 - DNA, 25 bp, Artificial
  Probe
tgggctaaacccagatggtttccta

SEQ ID NO: 461 - DNA, 25 bp, Artificial
  Probe
cccagatggtttcctaggaaatcac

SEQ ID NO: 462 - DNA, 25 bp, Artificial
  Probe
acaggcttctgagcacagctgcatt

SEQ ID NO: 463 - DNA, 25 bp, Artificial
  Probe
ctatgtgctccagggggacccaaga

SEQ ID NO: 464 - DNA, 25 bp, Artificial
  Probe
ccagtctggcaccagcgatcaggtc

SEQ ID NO: 465 - DNA, 25 bp, Artificial
  Probe
gatcaggtcctttatgggcagctgc

SEQ ID NO: 466 - DNA, 25 bp, Artificial
  Probe
ccccaagtcctatgagaacctctgg

SEQ ID NO: 467 - DNA, 25 bp, Artificial
  Probe
atgagaacctctggttccaggccag

SEQ ID NO: 468 - DNA, 25 bp, Artificial
  Probe
ggaggacgactgtgtctttgggcca

SEQ ID NO: 469 - DNA, 25 bp, Artificial
  Probe
ggatccgggtccatgggatggaggc

SEQ ID NO: 470 - DNA, 25 bp, Artificial
  Probe
gggcctgcctcttaaaggcctgagc

SEQ ID NO: 471 - DNA, 25 bp, Artificial
  Probe
ggagggtccataagcccatgactaa

SEQ ID NO: 472 - DNA, 25 bp, Artificial
  Probe
ctcccaggcgatctgcatactttaa

SEQ ID NO: 473 - DNA, 25 bp, Artificial
  Probe
ctttaaggaccagatcatgctccat

SEQ ID NO: 474 - DNA, 25 bp, Artificial
  Probe
gactcagcctctgggatggattact

SEQ ID NO: 475 - DNA, 25 bp, Artificial
  Probe
ggtgagaatggctacttccggatcc

SEQ ID NO: 476 - DNA, 25 bp, Artificial
  Probe
acttccggatccgcagaggaactga

SEQ ID NO: 477 - DNA, 25 bp, Artificial
  Probe
gagagcatagcagtggcagccacac

SEQ ID NO: 478 - DNA, 25 bp, Artificial
  Probe
gcagccacaccaattcctaaattgt

SEQ ID NO: 479 - DNA, 25 bp, Artificial
  Probe
gtagggtatgccttccagtatttca

SEQ ID NO: 480 - DNA, 25 bp, Artificial
  Probe
gccttccagtatttcataatgatct

SEQ ID NO: 481 - DNA, 25 bp, Artificial
  Probe
gatctgcatcagttgtaaaggggaa

SEQ ID NO: 482 - DNA, 25 bp, Artificial
  Probe
aattggtatattcacagactgtaga

SEQ ID NO: 483 - DNA, 25 bp, Artificial
  Probe
gactgtagactttcagcagcaatct

SEQ ID NO: 484 - DNA, 25 bp, Artificial
  Probe
acctttcaatcggccactggccatt

SEQ ID NO: 485 - DNA, 25 bp, Artificial
  Probe
gttataatctcttcctagctaatgg

SEQ ID NO: 486 - DNA, 25 bp, Artificial
  Probe
ctcttcctagctaatgggcttactc

SEQ ID NO: 487 - DNA, 25 bp, Artificial
  Probe
cttcctagctaatgggcttactcaa

SEQ ID NO: 488 - DNA, 25 bp, Artificial
  Probe
tagctaatgggcttactcaaagatt

SEQ ID NO: 489 - DNA, 25 bp, Artificial
  Probe
tgggcttactcaaagattcaccacc

SEQ ID NO: 490 - DNA, 25 bp, Artificial
  Probe
ctagcaatgatattctcagttgttt

SEQ ID NO: 491 - DNA, 25 bp, Artificial
  Probe
agcaatgatattctcagttgtttct

SEQ ID NO: 492 - DNA, 25 bp, Artificial
  Probe
gcaatgatattctcagttgtttctc

SEQ ID NO: 493 - DNA, 25 bp, Artificial
  Probe
ctcagttgtttctctcttgtggtgc

SEQ ID NO: 494 - DNA, 25 bp, Artificial
  Probe
ttctctcttgtggtgcagagttgca

SEQ ID NO: 495 - DNA, 25 bp, Artificial
  Probe
tctcttgtggtgcagagttgcattg

SEQ ID NO: 496 - DNA, 25 bp, Artificial
  Probe
ctcttgtggtgcagagttgcattgg

SEQ ID NO: 497 - DNA, 25 bp, Artificial
  Probe
tgcagagttgcattgggttttctac

SEQ ID NO: 498 - DNA, 25 bp, Artificial
  Probe
tgcattgggttttctacattttccc

SEQ ID NO: 499 - DNA, 25 bp, Artificial
  Probe
gcattgggttttctacattttccca

SEQ ID NO: 500 - DNA, 25 bp, Artificial
  Probe
cccactgagtcttccctgttgtaaa

SEQ ID NO: 501 - DNA, 25 bp, Artificial
  Probe
tgcttcctctctagaatccaattag

SEQ ID NO: 502 - DNA, 25 bp, Artificial
  Probe
agggatgtttgttactactcatatt

SEQ ID NO: 503 - DNA, 25 bp, Artificial
  Probe
atgtgagatcagtgaactctggttt

SEQ ID NO: 504 - DNA, 25 bp, Artificial
  Probe
gaactctggttttaagataatctga

SEQ ID NO: 505 - DNA, 25 bp, Artificial
  Probe
gataatctgaaacaaggtccttggg

SEQ ID NO: 506 - DNA, 25 bp, Artificial
  Probe
aaaattggtcacattctgtaaagca

SEQ ID NO: 507 - DNA, 25 bp, Artificial
  Probe
gtttaggaatcaacttatctcaaat

SEQ ID NO: 508 - DNA, 25 bp, Artificial
  Probe
tatctcaaattgtaactcggggcct

SEQ ID NO: 509 - DNA, 25 bp, Artificial
  Probe
tcaaattgtaactcggggcctaact

SEQ ID NO: 510 - DNA, 25 bp, Artificial
  Probe
ttcactaggtgatgccaaaatattt

SEQ ID NO: 511 - DNA, 25 bp, Artificial
  Probe
tgttaaactctaattgtgaaggcag

SEQ ID NO: 512 - DNA, 25 bp, Artificial
  Probe
tgaggaacccaatgaatgtgacttc

SEQ ID NO: 513 - DNA, 25 bp, Artificial
  Probe
agaatatggatagtttaccttctgg

SEQ ID NO: 514 - DNA, 25 bp, Artificial
  Probe
gaatttgtagttcaccggtacagtc

SEQ ID NO: 515 - DNA, 25 bp, Artificial
  Probe
ttcaccggtacagtctttactagac

SEQ ID NO: 516 - DNA, 25 bp, Artificial
  Probe
tctttactagacttgtttcagacta

SEQ ID NO: 517 - DNA, 25 bp, Artificial
  Probe
gaaatcagaatttttgggtttcaca

SEQ ID NO: 518 - DNA, 25 bp, Artificial
  Probe
tctgttaacagcgtttttctcgtcc

SEQ ID NO: 519 - DNA, 25 bp, Artificial
  Probe
cccttcaacttctacatttactggc

SEQ ID NO: 520 - DNA, 25 bp, Artificial
  Probe
caacttctacatttactggctttta

SEQ ID NO: 521 - DNA, 25 bp, Artificial
  Probe
ttaccagctttgtttacagacccaa

SEQ ID NO: 522 - DNA, 25 bp, Artificial
  Probe
taaacttgtgactggtcttgtttta

SEQ ID NO: 523 - DNA, 25 bp, Artificial
  Probe
agcccaaaggtttgcccgaatggag

SEQ ID NO: 524 - DNA, 25 bp, Artificial
  Probe
gaaacgctcgcgagaggatgctgga

SEQ ID NO: 525 - DNA, 25 bp, Artificial
  Probe
gagggtgctattggtgtcaggaaca

SEQ ID NO: 526 - DNA, 25 bp, Artificial
  Probe
aattgtgtccagaatgtgctcagct

SEQ ID NO: 527 - DNA, 25 bp, Artificial
  Probe
tcagctaattcagtattcttcccca

SEQ ID NO: 528 - DNA, 25 bp, Artificial
  Probe
gttactgttcttcgactttgattcc

SEQ ID NO: 529 - DNA, 25 bp, Artificial
  Probe
gactttgattccttgctcatgacat

SEQ ID NO: 530 - DNA, 25 bp, Artificial
  Probe
catgagtagggtgtgctcttctgtc

SEQ ID NO: 531 - DNA, 25 bp, Artificial
  Probe
ctgtcacttcacacagaccttttgc

SEQ ID NO: 532 - DNA, 25 bp, Artificial
  Probe
gatgatgcccatgacctgtaattgt

SEQ ID NO: 533 - DNA, 25 bp, Artificial
  Probe
atttaaaccatcttggcttgtgctt

SEQ ID NO: 534 - DNA, 25 bp, Artificial
  Probe
gccccggagcagattgcagcggaca

SEQ ID NO: 535 - DNA, 25 bp, Artificial
  Probe
cggagcagattgcagcggacatccc

SEQ ID NO: 536 - DNA, 25 bp, Artificial
  Probe
gattgcagcggacatcccagaagtg

SEQ ID NO: 537 - DNA, 25 bp, Artificial
  Probe
cagaagtggttgtttcccttgcatg

SEQ ID NO: 538 - DNA, 25 bp, Artificial
  Probe
agtggttgtttcccttgcatgggac

SEQ ID NO: 539 - DNA, 25 bp, Artificial
  Probe
ggttgtttcccttgcatgggacgaa

SEQ ID NO: 540 - DNA, 25 bp, Artificial
  Probe
gtttcccttgcatgggacgaaagct

SEQ ID NO: 541 - DNA, 25 bp, Artificial
  Probe
gcatgggacgaaagcttggctccaa

SEQ ID NO: 542 - DNA, 25 bp, Artificial
  Probe
gacgaaagcttggctccaaagcatc

SEQ ID NO: 543 - DNA, 25 bp, Artificial
  Probe
gcctagctagaggatctgtgacccc

SEQ ID NO: 544 - DNA, 25 bp, Artificial
  Probe
ctagctagaggatctgtgaccccag

SEQ ID NO: 545 - DNA, 25 bp, Artificial
  Probe
gcagcgtgggccagaagaccaggac

SEQ ID NO: 546 - DNA, 25 bp, Artificial
  Probe
aaaagctctgctcttcaggtttcag

SEQ ID NO: 547 - DNA, 25 bp, Artificial
  Probe
ggctcaacaaggggcatggtctgct

SEQ ID NO: 548 - DNA, 25 bp, Artificial
  Probe
tgggaactgcctcattggtggtgtg

SEQ ID NO: 549 - DNA, 25 bp, Artificial
  Probe
gtgagtttcacatactgctgcacgc

SEQ ID NO: 550 - DNA, 25 bp, Artificial
  Probe
gctgcacgcgtgtcgattaacgttc

SEQ ID NO: 551 - DNA, 25 bp, Artificial
  Probe
gattaacgttctgctgtccaagaga

SEQ ID NO: 552 - DNA, 25 bp, Artificial
  Probe
tcatgctgggaacgccatcatcggt

SEQ ID NO: 553 - DNA, 25 bp, Artificial
  Probe
ggtggtgttagcttcacatgcttct

SEQ ID NO: 554 - DNA, 25 bp, Artificial
  Probe
cacatgcttctgcagctgagcttgc

SEQ ID NO: 555 - DNA, 25 bp, Artificial
  Probe
cagccatgaggattatcgccctcct

SEQ ID NO: 556 - DNA, 25 bp, Artificial
  Probe
tgtgggaaggggcttctcatccact

SEQ ID NO: 557 - DNA, 25 bp, Artificial
  Probe
ccatcgtcatcgcttacttgatgaa

SEQ ID NO: 558 - DNA, 25 bp, Artificial
  Probe
gaagcacactcggatgaccatgact

SEQ ID NO: 559 - DNA, 25 bp, Artificial
  Probe
ggatgaccatgactgatgcttataa

SEQ ID NO: 560 - DNA, 25 bp, Artificial
  Probe
caaaggcaaacgaccaattatctcc

SEQ ID NO: 561 - DNA, 25 bp, Artificial
  Probe
ggggcagttgctagagttcgaggaa

SEQ ID NO: 562 - DNA, 25 bp, Artificial
  Probe
tgacaccgagaatccttacaccaaa

SEQ ID NO: 563 - DNA, 25 bp, Artificial
  Probe
tacaccaaagctgatgggcgtggag

SEQ ID NO: 564 - DNA, 25 bp, Artificial
  Probe
ggcgtggagacggttgtgtgacaat

SEQ ID NO: 565 - DNA, 25 bp, Artificial
  Probe
aaggattgctgctctccattaggag

SEQ ID NO: 566 - DNA, 25 bp, Artificial
  Probe
tgatgccattgagattcacctccca

SEQ ID NO: 567 - DNA, 25 bp, Artificial
  Probe
agacattgaatcaccaaggcctggg

SEQ ID NO: 568 - DNA, 25 bp, Artificial
  Probe
caaggcctgggatcaacctgggctg

SEQ ID NO: 569 - DNA, 25 bp, Artificial
  Probe
aaccaaaccaagccctgttgtgctc

SEQ ID NO: 570 - DNA, 25 bp, Artificial
  Probe
gatcagggcagcttaagtggtctaa

SEQ ID NO: 571 - DNA, 25 bp, Artificial
  Probe
gtggtctaagaatccttcaggcatt

SEQ ID NO: 572 - DNA, 25 bp, Artificial
  Probe
ggatacctttgattttgtgtgtttc

SEQ ID NO: 573 - DNA, 25 bp, Artificial
  Probe
gtgtgtttcatgctctggatttttt

SEQ ID NO: 574 - DNA, 25 bp, Artificial
  Probe
ggaactgaccattatatgccttcac

SEQ ID NO: 575 - DNA, 25 bp, Artificial
  Probe
cattatatgccttcactggcttctt

SEQ ID NO: 576 - DNA, 25 bp, Artificial
  Probe
atatatcaaatactttccttcccac

SEQ ID NO: 577 - DNA, 25 bp, Artificial
  Probe
gttacagtgccataaaccttgttac

SEQ ID NO: 578 - DNA, 25 bp, Artificial
  Probe
gtaagcttagtagttgcagaaattg

SEQ ID NO: 579 - DNA, 25 bp, Artificial
  Probe
gaacactaggtggcactcagttatc

SEQ ID NO: 580 - DNA, 25 bp, Artificial
  Probe
gtggcactcagttatcttaacaggg

SEQ ID NO: 581 - DNA, 25 bp, Artificial
  Probe
actgatacaattgttgacttttctt

SEQ ID NO: 582 - DNA, 25 bp, Artificial
  Probe
tactatgtgtaagaaataccccaaa

SEQ ID NO: 583 - DNA, 25 bp, Artificial
  Probe
gaaataccccaaacatgaaaagatt

SEQ ID NO: 584 - DNA, 25 bp, Artificial
  Probe
attgttttgatcatatgcatgtatg

SEQ ID NO: 585 - DNA, 25 bp, Artificial
  Probe
gaagtcatatacatgtaagctacaa

SEQ ID NO: 586 - DNA, 25 bp, Artificial
  Probe
gaaaagcctttttcaacatatccct

SEQ ID NO: 587 - DNA, 25 bp, Artificial
  Probe
tttttcaacatatccctaagctaag

SEQ ID NO: 588 - DNA, 25 bp, Artificial
  Probe
caactcagtgaaaagatggtctcca

SEQ ID NO: 589 - DNA, 25 bp, Artificial
  Probe
gaagaaggaaagtccccctgtgtgg

SEQ ID NO: 590 - DNA, 25 bp, Artificial
  Probe
ggaatctgcactattttggaggaca

SEQ ID NO: 591 - DNA, 25 bp, Artificial
  Probe
atgtccgtagttttatagtcctatt

SEQ ID NO: 592 - DNA, 25 bp, Artificial
  Probe
tatagtcctatttgtagcattcaat

SEQ ID NO: 593 - DNA, 25 bp, Artificial
  Probe
atagctttattccttagatggttct

SEQ ID NO: 594 - DNA, 25 bp, Artificial
  Probe
gatggttctagggtgggtttacagc

SEQ ID NO: 595 - DNA, 25 bp, Artificial
  Probe
actaacttcttcaactatggacttt

SEQ ID NO: 596 - DNA, 25 bp, Artificial
  Probe
tgtaatcctgtaggttggtacttcc

SEQ ID NO: 597 - DNA, 25 bp, Artificial
  Probe
tcccccaaactgattataggtaaca

SEQ ID NO: 598 - DNA, 25 bp, Artificial
  Probe
ggtaacagtttaatcatctcacttg

SEQ ID NO: 599 - DNA, 25 bp, Artificial
  Probe
atcatctcacttgctaacatgtttt

SEQ ID NO: 600 - DNA, 25 bp, Artificial
  Probe
gtgcagcgcatcttcgaaaacggct

SEQ ID NO: 601 - DNA, 25 bp, Artificial
  Probe
accccgtaaacttgctcaacgacat

SEQ ID NO: 602 - DNA, 25 bp, Artificial
  Probe
tcaacgacatcgtgattctccagct

SEQ ID NO: 603 - DNA, 25 bp, Artificial
  Probe
accatcaacgccaacgtgcaggtgg

SEQ ID NO: 604 - DNA, 25 bp, Artificial
  Probe
tgggcaggaaccgtgggatcgccag

SEQ ID NO: 605 - DNA, 25 bp, Artificial
  Probe
gcaggagctcaacgtgacggtggtg

SEQ ID NO: 606 - DNA, 25 bp, Artificial
  Probe
caacgtgacggtggtgacgtccctc

SEQ ID NO: 607 - DNA, 25 bp, Artificial
  Probe
cccttggtctgcaacgggctaatcc

SEQ ID NO: 608 - DNA, 25 bp, Artificial
  Probe
cgggctaatccacggaattgcctcc

SEQ ID NO: 609 - DNA, 25 bp, Artificial
  Probe
gccccggtggcacagtttgtaaact

SEQ ID NO: 610 - DNA, 25 bp, Artificial
  Probe
ttgtaaactggatcgactctatcat

SEQ ID NO: 611 - DNA, 25 bp, Artificial
  Probe
aaattcgttttgcaaatcattcggt

SEQ ID NO: 612 - DNA, 25 bp, Artificial
  Probe
aaatcattcggtaaatccaaactgc

SEQ ID NO: 613 - DNA, 25 bp, Artificial
  Probe
gatcacaggataggtatttttgcca

SEQ ID NO: 614 - DNA, 25 bp, Artificial
  Probe
ttttgccaagagagccatccagact

SEQ ID NO: 615 - DNA, 25 bp, Artificial
  Probe
ccatccagactggcgaagagctgtt

SEQ ID NO: 616 - DNA, 25 bp, Artificial
  Probe
gaaacagctgccttagcttcaggaa

SEQ ID NO: 617 - DNA, 25 bp, Artificial
  Probe
ctgccttagcttcaggaacctcgag

SEQ ID NO: 618 - DNA, 25 bp, Artificial
  Probe
tcaggaacctcgagtactgtgggca

SEQ ID NO: 619 - DNA, 25 bp, Artificial
  Probe
gccttctcaccagctgcaaagtgtt

SEQ ID NO: 620 - DNA, 25 bp, Artificial
  Probe
caaagtgttttgtaccagtgaattt

SEQ ID NO: 621 - DNA, 25 bp, Artificial
  Probe
gcagtatggtacatttttcaacttt

SEQ ID NO: 622 - DNA, 25 bp, Artificial
  Probe
gatagcacattcagtagccttattt

SEQ ID NO: 623 - DNA, 25 bp, Artificial
  Probe
tactgtatcatatgctcaactctga

SEQ ID NO: 624 - DNA, 25 bp, Artificial
  Probe
aaccttgaacacggccaaaatccat

SEQ ID NO: 625 - DNA, 25 bp, Artificial
  Probe
caattcaaactgacctgcatccatc

SEQ ID NO: 626 - DNA, 25 bp, Artificial
  Probe
ctgcatccatccaaaacaaattcct

SEQ ID NO: 627 - DNA, 25 bp, Artificial
  Probe
gagttaataccactggctcagcaaa

SEQ ID NO: 628 - DNA, 25 bp, Artificial
  Probe
aggaggccctttattattgctgcag

SEQ ID NO: 629 - DNA, 25 bp, Artificial
  Probe
gcctggctgagttgatgttttacat

SEQ ID NO: 630 - DNA, 25 bp, Artificial
  Probe
tctcccttactgaaatctacatgac

SEQ ID NO: 631 - DNA, 25 bp, Artificial
  Probe
gatgcttcttgctgggtttttgtac

SEQ ID NO: 632 - DNA, 25 bp, Artificial
  Probe
atggctggaggtgtgctttgtgtga

SEQ ID NO: 633 - DNA, 25 bp, Artificial
  Probe
gttgctgatttagagtcaatctcca

SEQ ID NO: 634 - DNA, 25 bp, Artificial
  Probe
tagagtcaatctccaatgttgtgct

SEQ ID NO: 635 - DNA, 25 bp, Artificial
  Probe
gggataagtcttatgctatctcagt

SEQ ID NO: 636 - DNA, 25 bp, Artificial
  Probe
tatgctatctcagttgacacattga

SEQ ID NO: 637 - DNA, 25 bp, Artificial
  Probe
cagttgacacattgaggttattttg

SEQ ID NO: 638 - DNA, 25 bp, Artificial
  Probe
gaagctagttggactttgttttgtt

SEQ ID NO: 639 - DNA, 25 bp, Artificial
  Probe
tgttttccaaaagttctccactatt

SEQ ID NO: 640 - DNA, 25 bp, Artificial
  Probe
aagttctccactattggttttagag

SEQ ID NO: 641 - DNA, 25 bp, Artificial
  Probe
agcaaggacatctttcctctgacac

SEQ ID NO: 642 - DNA, 25 bp, Artificial
  Probe
acgtgggaatgggtgatatttgtgt

SEQ ID NO: 643 - DNA, 25 bp, Artificial
  Probe
gaaatagcctccaatgggaaatatt

SEQ ID NO: 644 - DNA, 25 bp, Artificial
  Probe
ggagagaccccttcagagcagggat

SEQ ID NO: 645 - DNA, 25 bp, Artificial
  Probe
gaccccttcagagcagggattgtgc

SEQ ID NO: 646 - DNA, 25 bp, Artificial
  Probe
agggattgtgccgggagagtgcctc

SEQ ID NO: 647 - DNA, 25 bp, Artificial
  Probe
tttgggacatttcatccacagaaat

SEQ ID NO: 648 - DNA, 25 bp, Artificial
  Probe
cagaaatttccaagccaatggtttc

SEQ ID NO: 649 - DNA, 25 bp, Artificial
  Probe
cagcagatggaccatgcccttgctg

SEQ ID NO: 650 - DNA, 25 bp, Artificial
  Probe
gaaagaagtgtctctgttgggggac

SEQ ID NO: 651 - DNA, 25 bp, Artificial
  Probe
ttgggggacagaggaacctggggag

SEQ ID NO: 652 - DNA, 25 bp, Artificial
  Probe
gcatgtcctacaatctgctcttaga

SEQ ID NO: 653 - DNA, 25 bp, Artificial
  Probe
ctcttagacacggccttgccaggag

SEQ ID NO: 654 - DNA, 25 bp, Artificial
  Probe
ttagacacggccttgccaggagagc

SEQ ID NO: 655 - DNA, 25 bp, Artificial
  Probe
gaaactatgtgactcattctgtgaa

SEQ ID NO: 656 - DNA, 25 bp, Artificial
  Probe
aagacttcttgcagttgtgagttat

SEQ ID NO: 657 - DNA, 25 bp, Artificial
  Probe
aggctaatccatttagtgattccta

SEQ ID NO: 658 - DNA, 25 bp, Artificial
  Probe
gaacgctagtggtttgtccttagac

SEQ ID NO: 659 - DNA, 25 bp, Artificial
  Probe
ctttatcgctaagaccttgacttta

SEQ ID NO: 660 - DNA, 25 bp, Artificial
  Probe
aaatttttcatcactacaaccttga

SEQ ID NO: 661 - DNA, 25 bp, Artificial
  Probe
taatttcaggtcttcaacatgatga

SEQ ID NO: 662 - DNA, 25 bp, Artificial
  Probe
gtcttcaacactatgcgctttatca

SEQ ID NO: 663 - DNA, 25 bp, Artificial
  Probe
tgcgctttatcatattattcacaga

SEQ ID NO: 664 - DNA, 25 bp, Artificial
  Probe
ttgtaaatactgcttctgttttgtt

SEQ ID NO: 665 - DNA, 25 bp, Artificial
  Probe
gtttctcctttatacacttgactgt

SEQ ID NO: 666 - DNA, 25 bp, Artificial
  Probe
tccatccaccaagattacacgacgc

SEQ ID NO: 667 - DNA, 25 bp, Artificial
  Probe
acacgacgcagaacttgatccgcat

SEQ ID NO: 668 - DNA, 25 bp, Artificial
  Probe
ggctctcttggccatactggttgaa

SEQ ID NO: 669 - DNA, 25 bp, Artificial
  Probe
ttggcacagccatacggcactgaac

SEQ ID NO: 670 - DNA, 25 bp, Artificial
  Probe
ggcactgaacaaggaagcctcggca

SEQ ID NO: 671 - DNA, 25 bp, Artificial
  Probe
ggaagcctcggcagatgtggctgaa

SEQ ID NO: 672 - DNA, 25 bp, Artificial
  Probe
agatgtggctgaaccgagctggagc

SEQ ID NO: 673 - DNA, 25 bp, Artificial
  Probe
gctgaaccgagctggagccaacaga

SEQ ID NO: 674 - DNA, 25 bp, Artificial
  Probe
ggagccaacagatgtgtcagccagg

SEQ ID NO: 675 - DNA, 25 bp, Artificial
  Probe
gtcagccaggattgacctttgcacg

SEQ ID NO: 676 - DNA, 25 bp, Artificial
  Probe
ccaggattgacctttgcacgaacac

SEQ ID NO: 677 - DNA, 25 bp, Artificial
  Probe
ttccgcggagtccatgggacctgat

SEQ ID NO: 678 - DNA, 25 bp, Artificial
  Probe
cgcggagtccatgggacctgattca

SEQ ID NO: 679 - DNA, 25 bp, Artificial
  Probe
gagtccatgggacctgattcaagac

SEQ ID NO: 680 - DNA, 25 bp, Artificial
  Probe
gtccatgggacctgattcaagacca

SEQ ID NO: 681 - DNA, 25 bp, Artificial
  Probe
tgattcaagaccagaccctgacggc

SEQ ID NO: 682 - DNA, 25 bp, Artificial
  Probe
atgtggactacagcaactgggctcc

SEQ ID NO: 683 - DNA, 25 bp, Artificial
  Probe
tggactacagcaactgggctccagg

SEQ ID NO: 684 - DNA, 25 bp, Artificial
  Probe
actacagcaactgggctccagggga

SEQ ID NO: 685 - DNA, 25 bp, Artificial
  Probe
agggcgaggactgcgtgatgatgcg

SEQ ID NO: 686 - DNA, 25 bp, Artificial
  Probe
ggcgaggactgcgtgatgatgcggg

SEQ ID NO: 687 - DNA, 25 bp, Artificial
  Probe
aggactgcgtgatgatgcggggctc

SEQ ID NO: 688 - DNA, 25 bp, Artificial
  Probe
tgctgggatgaccagcatcagcccc

SEQ ID NO: 689 - DNA, 25 bp, Artificial
  Probe
ttctgccttctccatgctgagaaca

SEQ ID NO: 690 - DNA, 25 bp, Artificial
  Probe
aaatcacctattcactgcttatgca

SEQ ID NO: 691 - DNA, 25 bp, Artificial
  Probe
gcttatgcagtcggaagctccagaa

SEQ ID NO: 692 - DNA, 25 bp, Artificial
  Probe
gaagaacaaagagcccaattaccag

SEQ ID NO: 693 - DNA, 25 bp, Artificial
  Probe
gaaccacattaagtctccattgttt

SEQ ID NO: 694 - DNA, 25 bp, Artificial
  Probe
ccattgttttgccttgggatttgag

SEQ ID NO: 695 - DNA, 25 bp, Artificial
  Probe
aagacgaagggatgctgcagttcca

SEQ ID NO: 696 - DNA, 25 bp, Artificial
  Probe
ggtcccaaatgactgactgcacctt

SEQ ID NO: 697 - DNA, 25 bp, Artificial
  Probe
atccacacagccaatacaattagtc

SEQ ID NO: 698 - DNA, 25 bp, Artificial
  Probe
gaaagacgctatgttacaggttaca

SEQ ID NO: 699 - DNA, 25 bp, Artificial
  Probe
ggacaagcctctggtcaaggtcaca

SEQ ID NO: 700 - DNA, 25 bp, Artificial
  Probe
catccaagcctgtgaccatcactgt

SEQ ID NO: 701 - DNA, 25 bp, Artificial
  Probe
tcccagctcttcaccgatggggatc

SEQ ID NO: 702 - DNA, 25 bp, Artificial
  Probe
tggtcactgggattgctgtagcggc

SEQ ID NO: 703 - DNA, 25 bp, Artificial
  Probe
tagcggccattgttgctgctgtagt

SEQ ID NO: 704 - DNA, 25 bp, Artificial
  Probe
aagcggatttcagccaattccactg

SEQ ID NO: 705 - DNA, 25 bp, Artificial
  Probe
tccactgatcctgtgaaggctgccc

SEQ ID NO: 706 - DNA, 25 bp, Artificial
  Probe
tgcccaatttgagatgctttcctgc

SEQ ID NO: 707 - DNA, 25 bp, Artificial
  Probe
gcagccacctggacgtcaaatgatt

SEQ ID NO: 708 - DNA, 25 bp, Artificial
  Probe
aacagctgacggcggctacatgact

SEQ ID NO: 709 - DNA, 25 bp, Artificial
  Probe
taaaaacatctacctgactcttcct

SEQ ID NO: 710 - DNA, 25 bp, Artificial
  Probe
gtgcatctgactgtgctttctgagt

SEQ ID NO: 711 - DNA, 25 bp, Artificial
  Probe
agaaaccatcgtgctgaggtgccac

SEQ ID NO: 712 - DNA, 25 bp, Artificial
  Probe
aggacaagcctctggtcaaggtcat

SEQ ID NO: 713 - DNA, 25 bp, Artificial
  Probe
acataggctacacgctgtactcatc

SEQ ID NO: 714 - DNA, 25 bp, Artificial
  Probe
catccaagcctgtgaccatcactgt

SEQ ID NO: 715 - DNA, 25 bp, Artificial
  Probe
tcccagctcttcaccgatggggatc

SEQ ID NO: 716 - DNA, 25 bp, Artificial
  Probe
gatggggatcattgtggctgtggtc

SEQ ID NO: 717 - DNA, 25 bp, Artificial
  Probe
tggtcactgggattgctgtagcggc

SEQ ID NO: 718 - DNA, 25 bp, Artificial
  Probe
tagcggccattgttgctgctgtagt

SEQ ID NO: 719 - DNA, 25 bp, Artificial
  Probe
aacagctgacggcggctacatgact

SEQ ID NO: 720 - DNA, 25 bp, Artificial
  Probe
taaaaacatctacctgactcttcct

SEQ ID NO: 721 - DNA, 25 bp, Artificial
  Probe
gtcttccaggggactctatcagaac

SEQ ID NO: 722 - DNA, 25 bp, Artificial
  Probe
gatgagccctctaatgctaggagta

SEQ ID NO: 723 - DNA, 25 bp, Artificial
  Probe
gggactgaggattggggtgggggtg

SEQ ID NO: 724 - DNA, 25 bp, Artificial
  Probe
acagaacaaaccctgtgtcactgtc

SEQ ID NO: 725 - DNA, 25 bp, Artificial
  Probe
gtgtcactgtcccaagttaagctaa

SEQ ID NO: 726 - DNA, 25 bp, Artificial
  Probe
gtgaacagaactatctcagcatcag

SEQ ID NO: 727 - DNA, 25 bp, Artificial
  Probe
tctgcttcaatgtctagttcctgta

SEQ ID NO: 728 - DNA, 25 bp, Artificial
  Probe
tcctgtatagctttgttcattgcat

SEQ ID NO: 729 - DNA, 25 bp, Artificial
  Probe
actgagcttcactgagttacgctgt

SEQ ID NO: 730 - DNA, 25 bp, Artificial
  Probe
tttcaaatccttcttcagtcagttc

SEQ ID NO: 731 - DNA, 25 bp, Artificial
  Probe
aaaaagctttagctgtctcctgttt

SEQ ID NO: 732 - DNA, 25 bp, Artificial
  Probe
ggtatcaactggagtgcggcgaagg

SEQ ID NO: 733 - DNA, 25 bp, Artificial
  Probe
cctccacatgcacctgctagtgggg

SEQ ID NO: 734 - DNA, 25 bp, Artificial
  Probe
acccacaagcgctgcgtcgtggaag

SEQ ID NO: 735 - DNA, 25 bp, Artificial
  Probe
gagggcaaccaccagtttgctaagt

SEQ ID NO: 736 - DNA, 25 bp, Artificial
  Probe
gggcagtgcgggtaattctctaacg

SEQ ID NO: 737 - DNA, 25 bp, Artificial
  Probe
ttctctaacgggccacaacaacaac

SEQ ID NO: 738 - DNA, 25 bp, Artificial
  Probe
gtgagttcttcgaattgtgctgaga

SEQ ID NO: 739 - DNA, 25 bp, Artificial
  Probe
tccagggagcctggtggtacgccga

SEQ ID NO: 740 - DNA, 25 bp, Artificial
  Probe
gactgtcatgcttcaaacctcaatg

SEQ ID NO: 741 - DNA, 25 bp, Artificial
  Probe
ctcaatggtctctacctcatgggac

SEQ ID NO: 742 - DNA, 25 bp, Artificial
  Probe
atgggaccccatgagagctatgcca

SEQ ID NO: 743 - DNA, 25 bp, Artificial
  Probe
acacagctaccaattctactttacc

SEQ ID NO: 744 - DNA, 25 bp, Artificial
  Probe
ctgggggacactttcgagctcccag

SEQ ID NO: 745 - DNA, 25 bp, Artificial
  Probe
agcttcgtctcaccttgagttaggc

SEQ ID NO: 746 - DNA, 25 bp, Artificial
  Probe
aggctgagcacaggcatttcctgct

SEQ ID NO: 747 - DNA, 25 bp, Artificial
  Probe
attttaggattacccactcatcaga

SEQ ID NO: 748 - DNA, 25 bp, Artificial
  Probe
atgtccatcgttgctgtcagttatg

SEQ ID NO: 749 - DNA, 25 bp, Artificial
  Probe
gcttattgccaccaagatccacaag

SEQ ID NO: 750 - DNA, 25 bp, Artificial
  Probe
aggcttgattaagtccagtcgtccc

SEQ ID NO: 751 - DNA, 25 bp, Artificial
  Probe
ggtggtggcccttatagccacagtc

SEQ ID NO: 752 - DNA, 25 bp, Artificial
  Probe
cagtggatgtgacaagtgccctggc

SEQ ID NO: 753 - DNA, 25 bp, Artificial
  Probe
aaccccatgctctatgtcttcatgg

SEQ ID NO: 754 - DNA, 25 bp, Artificial
  Probe
agaagagccctgttggtgctttacc

SEQ ID NO: 755 - DNA, 25 bp, Artificial
  Probe
gagtctcccgaggacacaaacaggc

SEQ ID NO: 756 - DNA, 25 bp, Artificial
  Probe
gtgtagggagagttctttcctgttt

SEQ ID NO: 757 - DNA, 25 bp, Artificial
  Probe
ccggaaggccactcatggccatgcc

SEQ ID NO: 758 - DNA, 25 bp, Artificial
  Probe
tcatggccatgccaggagctttctc

SEQ ID NO: 759 - DNA, 25 bp, Artificial
  Probe
cataaacgatctcttgagtctcttt

SEQ ID NO: 760 - DNA, 25 bp, Artificial
  Probe
tattccaccctttctggtgtctata

SEQ ID NO: 761 - DNA, 25 bp, Artificial
  Probe
tgagagaccctggacgtttttctgc

SEQ ID NO: 762 - DNA, 25 bp, Artificial
  Probe
ggctgtatgaaacttgacggcgctt

SEQ ID NO: 763 - DNA, 25 bp, Artificial
  Probe
gacggcgcttttgtaaggtgccacc

SEQ ID NO: 764 - DNA, 25 bp, Artificial
  Probe
gctattgatgtacacttcgcaacgg

SEQ ID NO: 765 - DNA, 25 bp, Artificial
  Probe
gtgacttgacatgtccaatttcatt

SEQ ID NO: 766 - DNA, 25 bp, Artificial
  Probe
aaaatctcagattgcttgcttacag

SEQ ID NO: 767 - DNA, 25 bp, Artificial
  Probe
tggacaaacgattccttttagagga

SEQ ID NO: 768 - DNA, 25 bp, Artificial
  Probe
gttttagtaatctaggctttgcctg

SEQ ID NO: 769 - DNA, 25 bp, Artificial
  Probe
ggattgattctagaacctttgtata

SEQ ID NO: 770 - DNA, 25 bp, Artificial
  Probe
gatagtatttctaactttcatttct

SEQ ID NO: 771 - DNA, 25 bp, Artificial
  Probe
gttcatgttctgctatgcaatcgtt

SEQ ID NO: 772 - DNA, 25 bp, Artificial
  Probe
tttttttagattttcctggatgtat

SEQ ID NO: 773 - DNA, 25 bp, Artificial
  Probe
gtagcagtagtttacagttctagca

SEQ ID NO: 774 - DNA, 25 bp, Artificial
  Probe
aaaacaagacccagcttattttctg

SEQ ID NO: 775 - DNA, 25 bp, Artificial
  Probe
agcttattttctgcttgctgtaaat

SEQ ID NO: 776 - DNA, 25 bp, Artificial
  Probe
attgagcccttactgtgggcaaatc

SEQ ID NO: 777 - DNA, 25 bp, Artificial
  Probe
gataattcccttattcagtaaatgt

SEQ ID NO: 778 - DNA, 25 bp, Artificial
  Probe
aaatgtctactgagcacaatctagt

SEQ ID NO: 779 - DNA, 25 bp, Artificial
  Probe
gtgaatcattacagtatggcctcat

SEQ ID NO: 780 - DNA, 25 bp, Artificial
  Probe
aacaatattttacaccattcgtatc

SEQ ID NO: 781 - DNA, 25 bp, Artificial
  Probe
gtaattgtgtggttatctgccattt

SEQ ID NO: 782 - DNA, 25 bp, Artificial
  Probe
aagtatccagtatttgatcacatta

SEQ ID NO: 783 - DNA, 25 bp, Artificial
  Probe
ctggtttattgtgcagtgactgtaa

SEQ ID NO: 784 - DNA, 25 bp, Artificial
  Probe
ctgcctcaccaaacacatgctagga

SEQ ID NO: 785 - DNA, 25 bp, Artificial
  Probe
catgctaggatataacccccaaaat

SEQ ID NO: 786 - DNA, 25 bp, Artificial
  Probe
gacagagagctgttatcctaactga

SEQ ID NO: 787 - DNA, 25 bp, Artificial
  Probe
ttccccatctgggccttcataaaat

SEQ ID NO: 788 - DNA, 25 bp, Artificial
  Probe
atgcaggggaagccagactggtctc

SEQ ID NO: 789 - DNA, 25 bp, Artificial
  Probe
tggtctcaggagcgctaaagccctt

SEQ ID NO: 790 - DNA, 25 bp, Artificial
  Probe
gccctgctgtttaggacctgggacc

SEQ ID NO: 791 - DNA, 25 bp, Artificial
  Probe
ctgggaccacaatggggtacctgcc

SEQ ID NO: 792 - DNA, 25 bp, Artificial
  Probe
tccccaagagatccaggctgtcatg

SEQ ID NO: 793 - DNA, 25 bp, Artificial
  Probe
gttggctacttgtgtcttgaaatct

SEQ ID NO: 794 - DNA, 25 bp, Artificial
  Probe
agtggaagcccagtcttgagttctt

SEQ ID NO: 795 - DNA, 25 bp, Artificial
  Probe
gagttcttgtcttgttaccatttaa

SEQ ID NO: 796 - DNA, 25 bp, Artificial
  Probe
agtgggtcatgtttttgctgtggtg

SEQ ID NO: 797 - DNA, 25 bp, Artificial
  Probe
gacacatggtacaggcttggagctt

SEQ ID NO: 798 - DNA, 25 bp, Artificial
  Probe
ggaacataacccaggagtctaagtt

SEQ ID NO: 799 - DNA, 25 bp, Artificial
  Probe
actgaacttgcaggtccaggttggt

SEQ ID NO: 800 - DNA, 25 bp, Artificial
  Probe
ccaggttggtatacattccaccctc

SEQ ID NO: 801 - DNA, 25 bp, Artificial
  Probe
ttccaccctctagaagtattttctt

SEQ ID NO: 802 - DNA, 25 bp, Artificial
  Probe
agataagctgctcacattttgtttt

SEQ ID NO: 803 - DNA, 25 bp, Artificial
  Probe
gttttgaatgggcatctcctgagga

SEQ ID NO: 804 - DNA, 25 bp, Artificial
  Probe
atctcctgaggaaatgtagcatgac

SEQ ID NO: 805 - DNA, 25 bp, Artificial
  Probe
agcatgacattggtactaactgcat

SEQ ID NO: 806 - DNA, 25 bp, Artificial
  Probe
ggtactaactgcatgtgtaaataca

SEQ ID NO: 807 - DNA, 25 bp, Artificial
  Probe
aatacatcatactggcaaaccgtaa

SEQ ID NO: 808 - DNA, 25 bp, Artificial
  Probe
gtatcatcattcatgtagtatctat

SEQ ID NO: 809 - DNA, 25 bp, Artificial
  Probe
gaaatttcctaactctatcagataa

SEQ ID NO: 810 - DNA, 25 bp, Artificial
  Probe
atttgcaggttgccacaggtggact

SEQ ID NO: 811 - DNA, 25 bp, Artificial
  Probe
gtaacctaacccatgtttcagcttc

SEQ ID NO: 812 - DNA, 25 bp, Artificial;
agtaactccagtagccttcattagt

SEQ ID NO: 813 - DNA, 25 bp, Artificial
  Probe
gcatgctgcttcgactctaaatatc

SEQ ID NO: 814 - DNA, 25 bp, Artificial
  Probe
taaatatctggttttccctgtcttt

SEQ ID NO: 815 - DNA, 25 bp, Artificial
  Probe
tttactacttccccagattcagaac

SEQ ID NO: 816 - DNA, 25 bp, Artificial
  Probe
ggggatctgattttagaggccttaa

SEQ ID NO: 817 - DNA, 25 bp, Artificial
  Probe
aggccttaattttctgttcatggac

SEQ ID NO: 818 - DNA, 25 bp, Artificial
  Probe
atgctgggacatcattactaaccaa

SEQ ID NO: 819 - DNA, 25 bp, Artificial
  Probe
gaacactcttctatgaacaaccacc

SEQ ID NO: 820 - DNA, 25 bp, Artificial
  Probe
aggctgtggtcatgcggaacactct

SEQ ID NO: 821 - DNA, 25 bp, Artificial
  Probe
ggctatccagataatcctgaacact

SEQ ID NO: 822 - DNA, 25 bp, Artificial
  Probe
cagccccctacaccaagagtgtatc

SEQ ID NO: 823 - DNA, 25 bp, Artificial
  Probe
gaaagagctcctacactttgaaaac

SEQ ID NO: 824 - DNA, 25 bp, Artificial
  Probe
cccttatcatgaagtttgcctgttc

SEQ ID NO: 825 - DNA, 25 bp, Artificial
  Probe
aatttccttcaatctctagtgacaa

SEQ ID NO: 826 - DNA, 25 bp, Artificial
  Probe
tactgcccattagctaaaatcattt

SEQ ID NO: 827 - DNA, 25 bp, Artificial
  Probe
ttctttctaactacatgcatctctc

SEQ ID NO: 828 - DNA, 25 bp, Artificial
  Probe
ccaccttgaaaatcgctgctctgaa

SEQ ID NO: 829 - DNA, 25 bp, Artificial
  Probe
aaatcgctgctctgaaccagtgttc

SEQ ID NO: 830 - DNA, 25 bp, Artificial
  Probe
ttggtcttggtgtcatatggatcag

SEQ ID NO: 831 - DNA, 25 bp, Artificial
  Probe
caagagatcctcagtcaattgccca

SEQ ID NO: 832 - DNA, 25 bp, Artificial
  Probe
gggcttctggaatttgtcgatatca

SEQ ID NO: 833 - DNA, 25 bp, Artificial
  Probe
atttgtcgatatcacagccaccaac

SEQ ID NO: 834 - DNA, 25 bp, Artificial
  Probe
ccaccaaccacactaacgagattca

SEQ ID NO: 835 - DNA, 25 bp, Artificial
  Probe
gattatttgcaacagctcacgggag

SEQ ID NO: 836 - DNA, 25 bp, Artificial
  Probe
caagaacggtgcctcgagtctttat

SEQ ID NO: 837 - DNA, 25 bp, Artificial
  Probe
cagtgatctagtctctttgcaacag

SEQ ID NO: 838 - DNA, 25 bp, Artificial
  Probe
acagagtggggaactgctgacgcgg

SEQ ID NO: 839 - DNA, 25 bp, Artificial
  Probe
tgtgaactgcaaaatccagcctggg

SEQ ID NO: 840 - DNA, 25 bp, Artificial
  Probe
gaaggtggttgtgttcatcaagccc

SEQ ID NO: 841 - DNA, 25 bp, Artificial
  Probe
ccacctgcccgtactgcaggagggc

SEQ ID NO: 842 - DNA, 25 bp, Artificial
  Probe
tgccagacccacaagccttgaagag

SEQ ID NO: 843 - DNA, 25 bp, Artificial
  Probe
gagagatgtcccctgtgataatgtc

SEQ ID NO: 844 - DNA, 25 bp, Artificial
  Probe
tgataatgtcagcagctgtccctcc

SEQ ID NO: 845 - DNA, 25 bp, Artificial
  Probe
gccgagacaaccgacagggctgggc

SEQ ID NO: 846 - DNA, 25 bp, Artificial
  Probe
cgcagccaggggtaccaagtgtttg

SEQ ID NO: 847 - DNA, 25 bp, Artificial
  Probe
taccaagtgtttgcgcagggaggcc

SEQ ID NO: 848 - DNA, 25 bp, Artificial
  Probe
gacccagccttgagacagctgctgt

SEQ ID NO: 849 - DNA, 25 bp, Artificial
  Probe
gagggacagtactgaagactctgca

SEQ ID NO: 850 - DNA, 25 bp, Artificial
  Probe
tggggcctcaatctaaggccttccc

SEQ ID NO: 851 - DNA, 25 bp, Artificial
  Probe
aaagccacattacaagctgccatcc

SEQ ID NO: 852 - DNA, 25 bp, Artificial
  Probe
ttttccctatccacaggggtgtttg

SEQ ID NO: 853 - DNA, 25 bp, Artificial
  Probe
gagaaagaagtggtctctgcccagc

SEQ ID NO: 854 - DNA, 25 bp, Artificial
  Probe
cgtagccctcacgtgggtgtgaagg

SEQ ID NO: 855 - DNA, 25 bp, Artificial
  Probe
gaaggacacttctgccatgataacc

SEQ ID NO: 856 - DNA, 25 bp, Artificial
  Probe
tgccatgataaccagacctgctgcc

SEQ ID NO: 857 - DNA, 25 bp, Artificial
  Probe
gccgagacaaccgacagggctgggc

SEQ ID NO: 858 - DNA, 25 bp, Artificial
  Probe
gccaggggtaccaagtgtttgcgca

SEQ ID NO: 859 - DNA, 25 bp, Artificial
  Probe
gacccagccttgagacagctgctgt

SEQ ID NO: 860 - DNA, 25 bp, Artificial
  Probe
cagtactgaagactctgcagccctc

SEQ ID NO: 861 - DNA, 25 bp, Artificial
  Probe
tgagctccccatcaccatgggaggt

SEQ ID NO: 862 - DNA, 25 bp, Artificial
  Probe
tggggcctcaatctaaggccttccc

SEQ ID NO: 863 - DNA, 25 bp, Artificial
  Probe
aaagccacattacaagctgccatcc

SEQ ID NO: 864 - DNA, 25 bp, Artificial
  Probe
gatagatggtgcagcatgtctacat

SEQ ID NO: 865 - DNA, 25 bp, Artificial
  Probe
gcatgtctacatggttgtttgttgc

SEQ ID NO: 866 - DNA, 25 bp, Artificial
  Probe
taatgtgtggtttcaattcagcttg

SEQ ID NO: 867 - DNA, 25 bp, Artificial
  Probe
gaaaaataatctcactacatgtagc

SEQ ID NO: 868 - DNA, 25 bp, Artificial
  Probe
atttctgctttgaatccttgatatt

SEQ ID NO: 869 - DNA, 25 bp, Artificial
  Probe
attgcaatggaattcctactttatt

SEQ ID NO: 870 - DNA, 25 bp, Artificial
  Probe
tatgctagttattgtgtgcgattta

SEQ ID NO: 871 - DNA, 25 bp, Artificial
  Probe
ttttggttgtgcgctttcttttaca

SEQ ID NO: 872 - DNA, 25 bp, Artificial
  Probe
tttacaacaagcctctagaaacaga

SEQ ID NO: 873 - DNA, 25 bp, Artificial
  Probe
gtttctgagaattactgagctatgt

SEQ ID NO: 874 - DNA, 25 bp, Artificial
  Probe
ggattcaatgtttgtctttggtttt

SEQ ID NO: 875 - DNA, 25 bp, Artificial
  Probe
ttcaggtgttactcagctgcatagt

SEQ ID NO: 876 - DNA, 25 bp, Artificial
  Probe
gtgttactcagctgcatagttacgc

SEQ ID NO: 877 - DNA, 25 bp, Artificial
  Probe
agttacgcagatgtaatgcacatta

SEQ ID NO: 878 - DNA, 25 bp, Artificial
  Probe
ttggcgtatctttaagttggattca

SEQ ID NO: 879 - DNA, 25 bp, Artificial
  Probe
ttaagttggattcaaatggccattt

SEQ ID NO: 880 - DNA, 25 bp, Artificial
  Probe
gatgctgtctatttgcattgagtgt

SEQ ID NO: 881 - DNA, 25 bp, Artificial
  Probe
gcattgagtgtaagtcatttgaact

SEQ ID NO: 882 - DNA, 25 bp, Artificial
  Probe
aactgggaacataaagtgcctgtat

SEQ ID NO: 883 - DNA, 25 bp, Artificial
  Probe
caaagtgtacgtgaatgctcgctgt

SEQ ID NO: 884 - DNA, 25 bp, Artificial
  Probe
agggttccagctccatatatataga

SEQ ID NO: 885 - DNA, 25 bp, Artificial
  Probe
gagccccatccagttagttggacta

SEQ ID NO: 886 - DNA, 25 bp, Artificial
  Probe
gggaggactataaggccatggcccg

SEQ ID NO: 887 - DNA, 25 bp, Artificial
  Probe
taaggccatggcccgtgatgagaag

SEQ ID NO: 888 - DNA, 25 bp, Artificial
  Probe
ggccatggcccgtgatgagaagaat

SEQ ID NO: 889 - DNA, 25 bp, Artificial
  Probe
ggcccgtgatgagaagaattactat

SEQ ID NO: 890 - DNA, 25 bp, Artificial
  Probe
gaattactatcaagataccccaaaa

SEQ ID NO: 891 - DNA, 25 bp, Artificial
  Probe
gataccccaaaacagattcggagta

SEQ ID NO: 892 - DNA, 25 bp, Artificial
  Probe
tcggagtaagatcaacgtctataaa

SEQ ID NO: 893 - DNA, 25 bp, Artificial
  Probe
gatcaacgtctataaacgcttttac

SEQ ID NO: 894 - DNA, 25 bp, Artificial
  Probe
gtctataaacgcttttacccagcag

SEQ ID NO: 895 - DNA, 25 bp, Artificial
  Probe
aacgcttttacccagcagagtggca

SEQ ID NO: 896 - DNA, 25 bp, Artificial
  Probe
tttacccagcagagtggcaagactt

SEQ ID NO: 897 - DNA, 25 bp, Artificial
  Probe
tcccgacactaactatactctctac

SEQ ID NO: 898 - DNA, 25 bp, Artificial
  Probe
actatactctctactattggcacag

SEQ ID NO: 899 - DNA, 25 bp, Artificial
  Probe
gaaggccaatactttggttgttcct

SEQ ID NO: 900 - DNA, 25 bp, Artificial
  Probe
actttggttgttcctttgatctgac

SEQ ID NO: 901 - DNA, 25 bp, Artificial
  Probe
ggattccagttttgaacaacacagt

SEQ ID NO: 902 - DNA, 25 bp, Artificial
  Probe
aaaccatccttcaatatagtgcctt

SEQ ID NO: 903 - DNA, 25 bp, Artificial
  Probe
aatatagtgcctttaacttcccgtg

SEQ ID NO: 904 - DNA, 25 bp, Artificial
  Probe
taacttcccgtgtgaaacctgatcc

SEQ ID NO: 905 - DNA, 25 bp, Artificial
  Probe
aacctgatcctccacatattaaaaa

SEQ ID NO: 906 - DNA, 25 bp, Artificial
  Probe
ctccttccacaatgatgacctatat

SEQ ID NO: 907 - DNA, 25 bp, Artificial
  Probe
gatgacctatatgtgcaatgggaga

SEQ ID NO: 908 - DNA, 25 bp, Artificial
  Probe
gagaggatttgcacatggattttaa

SEQ ID NO: 909 - DNA, 25 bp, Artificial
  Probe
gttgtccataataccctgagttttc

SEQ ID NO: 910 - DNA, 25 bp, Artificial
  Probe
tgagttttcagacactacgcaccac

SEQ ID NO: 911 - DNA, 25 bp, Artificial
  Probe
acgcaccacagtcaaggaagcctcc

SEQ ID NO: 912 - DNA, 25 bp, Artificial
  Probe
gggggaatatggatgcacagacggt

SEQ ID NO: 913 - DNA, 25 bp, Artificial
  Probe
cagacggtgcaaacacagaactgga

SEQ ID NO: 914 - DNA, 25 bp, Artificial
  Probe
gatggtctgactgtgctatggcctc

SEQ ID NO: 915 - DNA, 25 bp, Artificial
  Probe
tgctatggcctcatcatcaagactt

SEQ ID NO: 916 - DNA, 25 bp, Artificial
  Probe
atcaagactttcaatcctatcccaa

SEQ ID NO: 917 - DNA, 25 bp, Artificial
  Probe
gggccacgccaggaatattcagaaa

SEQ ID NO: 918 - DNA, 25 bp, Artificial
  Probe
gagaactacattgaagtgccattga

SEQ ID NO: 919 - DNA, 25 bp, Artificial
  Probe
atctcatacccgcaaattttaacct

SEQ ID NO: 920 - DNA, 25 bp, Artificial
  Probe
agtatgccctgacctgagtgaattc

SEQ ID NO: 921 - DNA, 25 bp, Artificial
  Probe
ggtacaaggattctcttcttttgga

SEQ ID NO: 922 - DNA, 25 bp, Artificial
  Probe
ccactcacttactcgtacacgatgt

SEQ ID NO: 923 - DNA, 25 bp, Artificial
  Probe
tattaccgctgtgtcctgacatttg

SEQ ID NO: 924 - DNA, 25 bp, Artificial
  Probe
gaaggccagcaatacaacatcacta

SEQ ID NO: 925 - DNA, 25 bp, Artificial
  Probe
aagaccatatcagcttctctggggt

SEQ ID NO: 926 - DNA, 25 bp, Artificial
  Probe
taaggtgtttctgggaaccggcaca

SEQ ID NO: 927 - DNA, 25 bp, Artificial
  Probe
acacccttaaccaccatgctgtggt

SEQ ID NO: 928 - DNA, 25 bp, Artificial
  Probe
tggtggacggccaatgacacccaca

SEQ ID NO: 929 - DNA, 25 bp, Artificial
  Probe
atgacacccacatagagagcgccta

SEQ ID NO: 930 - DNA, 25 bp, Artificial
  Probe
acctggagacagtggcggcttatta

SEQ ID NO: 931 - DNA, 25 bp, Artificial
  Probe
ggcttattatgaggagcagcaccca

SEQ ID NO: 932 - DNA, 25 bp, Artificial
  Probe
aagagatggattacggtgccgaggc

SEQ ID NO: 933 - DNA, 25 bp, Artificial
  Probe
tacggtgccgaggcaacagatcccc

SEQ ID NO: 934 - DNA, 25 bp, Artificial
  Probe
atcccctgtcccggatgttgaggat

SEQ ID NO: 935 - DNA, 25 bp, Artificial
  Probe
tcccggatgttgaggatcccgcaac

SEQ ID NO: 936 - DNA, 25 bp, Artificial
  Probe
cccgcaaccgaggagcctggggaga

SEQ ID NO: 937 - DNA, 25 bp, Artificial
  Probe
tgagatggttccaggccatgctgca

SEQ ID NO: 938 - DNA, 25 bp, Artificial
  Probe
ctgctctctgtcagagctcttcatg

SEQ ID NO: 939 - DNA, 25 bp, Artificial
  Probe
ctgacaccccagaagtgctctgaac

SEQ ID NO: 940 - DNA, 25 bp, Artificial
  Probe
atgaagatactgacaccacctttgc

SEQ ID NO: 941 - DNA, 25 bp, Artificial
  Probe
gatgggtcgtgtgatgagatgcatt

SEQ ID NO: 942 - DNA, 25 bp, Artificial
  Probe
gagatgcatttaaggccgatagtga

SEQ ID NO: 943 - DNA, 25 bp, Artificial
  Probe
ggccgatagtgatagatgttttttt

SEQ ID NO: 944 - DNA, 25 bp, Artificial
  Probe
tttcttgaacacaggctttgtctga

SEQ ID NO: 945 - DNA, 25 bp, Artificial
  Probe
gaatgatgttcttttatctcttgaa

SEQ ID NO: 946 - DNA, 25 bp, Artificial
  Probe
taagtgctgttacattaataccata

SEQ ID NO: 947 - DNA, 25 bp, Artificial
  Probe
tctctagtctcaatatgtatgtgta

SEQ ID NO: 948 - DNA, 25 bp, Artificial
  Probe
gtgcaatttgctagtaggacaatgc

SEQ ID NO: 949 - DNA, 25 bp, Artificial
  Probe
ggacaatgcagtgactgactagcat

SEQ ID NO: 950 - DNA, 25 bp, Artificial
  Probe
gtgcaatcctttcatgttcacttgc

SEQ ID NO: 951 - DNA, 25 bp, Artificial
  Probe
tattagctctaatcccttaagtaaa

SEQ ID NO: 952 - DNA, 25 bp, Artificial
  Probe
acctaacgaagtatccttcagcctg

SEQ ID NO: 953 - DNA, 25 bp, Artificial
  Probe
acatgttacaacacggacgaacctt

SEQ ID NO: 954 - DNA, 25 bp, Artificial
  Probe
ggacgaaccttgaaaactttatgct

SEQ ID NO: 955 - DNA, 25 bp, Artificial
  Probe
atagtttatgattccacctacatga

SEQ ID NO: 956 - DNA, 25 bp, Artificial
  Probe
gattaccagggactgaggggagggg

SEQ ID NO: 957 - DNA, 25 bp, Artificial
  Probe
ggggagcatgggaagtgacggttta

SEQ ID NO: 958 - DNA, 25 bp, Artificial
  Probe
aatgggcacagggtttatgtttagg

SEQ ID NO: 959 - DNA, 25 bp, Artificial
  Probe
acagtagtgatagttgtaccgcaat

SEQ ID NO: 960 - DNA, 25 bp, Artificial
  Probe
gtaccgcaatgtgacttaatgccac

SEQ ID NO: 961 - DNA, 25 bp, Artificial
  Probe
accaaggctgattaaaccaaggcta

SEQ ID NO: 962 - DNA, 25 bp, Artificial
  Probe
taaaccaaggctagaaccacctgcc

SEQ ID NO: 963 - DNA, 25 bp, Artificial
  Probe
acaggagtcagtgtctggctttttc

SEQ ID NO: 964 - DNA, 25 bp, Artificial
  Probe
tggctcctaggggaacagaccagtg

SEQ ID NO: 965 - DNA, 25 bp, Artificial
  Probe
taacaccaatcccagggctggctct

SEQ ID NO: 966 - DNA, 25 bp, Artificial
  Probe
ggctggctctgcactaagcgaaaat

SEQ ID NO: 967 - DNA, 25 bp, Artificial
  Probe
tccaaagaactaccccttttcagct

SEQ ID NO: 968 - DNA, 25 bp, Artificial
  Probe
ccctggggactgttccaaagccagt

SEQ ID NO: 969 - DNA, 25 bp, Artificial
  Probe
aacttggcactttttcgtgtggatc

SEQ ID NO: 970 - DNA, 25 bp, Artificial
  Probe
gtggatcttgccacatttctgatca

SEQ ID NO: 971 - DNA, 25 bp, Artificial
  Probe
cagaggtgtacactaacatttcccc

SEQ ID NO: 972 - DNA, 25 bp, Artificial
  Probe
ttatttatacagtgccttgctcggg

SEQ ID NO: 973 - DNA, 25 bp, Artificial
  Probe
gtgtgagcgccttggtatgacttaa

SEQ ID NO: 974 - DNA, 25 bp, Artificial
  Probe
gcacttgaaccagattatagattta

SEQ ID NO: 975 - DNA, 25 bp, Artificial
  Probe
aaactagaatagccagtatttatgt

SEQ ID NO: 976 - DNA, 25 bp, Artificial
  Probe
gtgcaatacgaattatgcaatcaca

SEQ ID NO: 977 - DNA, 25 bp, Artificial
  Probe
atcacaatacatttgtagctcccga

SEQ ID NO: 978 - DNA, 25 bp, Artificial
  Probe
aatacatttgtagctcccgagtgtc

SEQ ID NO: 979 - DNA, 25 bp, Artificial
  Probe
atttgtagctcccgagtgtcctaaa

SEQ ID NO: 980 - DNA, 25 bp, Artificial
  Probe
gtagctcccgagtgtcctaaaggga

SEQ ID NO: 981 - DNA, 25 bp, Artificial
  Probe
gtcctaaagggagtgcacttctttg

SEQ ID NO: 982 - DNA, 25 bp, Artificial
  Probe
acttctttgaagctggtgtgttaat

SEQ ID NO: 983 - DNA, 25 bp, Artificial
  Probe
gaagctggtgtgttaatactatgta

SEQ ID NO: 984 - DNA, 25 bp, Artificial
  Probe
taactttcaaatgatgctgctgcca

SEQ ID NO: 985 - DNA, 25 bp, Artificial
  Probe
tttgtttttctgctttagacttgaa

SEQ ID NO: 986 - DNA, 25 bp, Artificial
  Probe
gagacaggcaggtgatctgctgcag

SEQ ID NO: 987 - DNA, 25 bp, Artificial
  Probe
ggaagcacaccaatctgactttgta

SEQ ID NO: 988 - DNA, 25 bp, Artificial
  Probe
gatttcttttcaccattcgtacata

SEQ ID NO: 989 - DNA, 25 bp, Artificial
  Probe
gaaccacttgtagatttgatttttt

SEQ ID NO: 990 - DNA, 25 bp, Artificial
  Probe
agatcactgtttagatttgccatag

SEQ ID NO: 991 - DNA, 25 bp, Artificial
  Probe
tttgccatagagtacactgcctgcc

SEQ ID NO: 992 - DNA, 25 bp, Artificial
  Probe
gtacactgcctgccttaagtgagga

SEQ ID NO: 993 - DNA, 25 bp, Artificial
  Probe
agagtaatcttgttggttcaccatt

SEQ ID NO: 994 - DNA, 25 bp, Artificial
  Probe
gatactttgtattgtcctattagtg

SEQ ID NO: 995 - DNA, 25 bp, Artificial
  Probe
gcatctttgatgtgttgttcttggc

SEQ ID NO: 996 - DNA, 25 bp, Artificial
  Probe
gatttcttttcaccattcgtacata

SEQ ID NO: 997 - DNA, 25 bp, Artificial
  Probe
aatactgaaccacttgtagatttga

SEQ ID NO: 998 - DNA, 25 bp, Artificial
  Probe
aagctagttgaatacttgaaccata

SEQ ID NO: 999 - DNA, 25 bp, Artificial
  Probe
tttgccatagagtacactgcctgcc

SEQ ID NO: 1000 - DNA, 25 bp, Artificial
  Probe
gtacactgcctgccttaagtgagga

SEQ ID NO: 1001 - DNA, 25 bp, Artificial
  Probe
atcaaagtgctattacgaagttcaa

SEQ ID NO: 1002 - DNA, 25 bp, Artificial
  Probe
acagagtaatcttgttggttcacca

SEQ ID NO: 1003 - DNA, 25 bp, Artificial
  Probe
tgttggttcaccattgagaccgtga

SEQ ID NO: 1004 - DNA, 25 bp, Artificial
  Probe
gagaccgtgaagatactttgtattg

SEQ ID NO: 1005 - DNA, 25 bp, Artificial
  Probe
gatactttgtattgtcctattagtg

SEQ ID NO: 1006 - DNA, 25 bp, Artificial
  Probe
gcatctttgatgtgttgttcttggc

SEQ ID NO: 1007 - DNA, 25 bp, Artificial
  Probe
gtcagttcttatcaaaaagctcggt

SEQ ID NO: 1008 - DNA, 25 bp, Artificial
  Probe
aaagctcggtactgcactacaggat

SEQ ID NO: 1009 - DNA, 25 bp, Artificial
  Probe
gtctgttttattacactggagtgtt

SEQ ID NO: 1010 - DNA, 25 bp, Artificial
  Probe
gtaagttaacctgttctagttccat

SEQ ID NO: 1011 - DNA, 25 bp, Artificial
  Probe
ctgttctagttccatcattctgtgt

SEQ ID NO: 1012 - DNA, 25 bp, Artificial
  Probe
atgtgcaatacaattcctgcatctt

SEQ ID NO: 1013 - DNA, 25 bp, Artificial
  Probe
aattcctgcatctttaaaatgtctg

SEQ ID NO: 1014 - DNA, 25 bp, Artificial
  Probe
tattggattggccgtaacttttaga

SEQ ID NO: 1015 - DNA, 25 bp, Artificial
  Probe
gggaggtcattaattgctttttctt

SEQ ID NO: 1016 - DNA, 25 bp, Artificial
  Probe
acctgtttgtatatagcttgagtaa

SEQ ID NO: 1017 - DNA, 25 bp, Artificial
  Probe
attgtgatatgattgtataccacta

SEQ ID NO: 1018 - DNA, 25 bp, Artificial
  Probe
gtggcttttgtcaagcacttagatg

SEQ ID NO: 1019 - DNA, 25 bp, Artificial
  Probe
agatggatataaatgcagcaacttg

SEQ ID NO: 1020 - DNA, 25 bp, Artificial
  Probe
tgtaacgtataaactcaagcctttt

SEQ ID NO: 1021 - DNA, 25 bp, Artificial
  Probe
aatgtcacaaaacaggaaccagcat

SEQ ID NO: 1022 - DNA, 25 bp, Artificial
  Probe
gatatggttcaaataggactactag

SEQ ID NO: 1023 - DNA, 25 bp, Artificial
  Probe
aggactactagagttcattgaacac

SEQ ID NO: 1024 - DNA, 25 bp, Artificial
  Probe
gaaagactattgcaggtgtttaaaa

SEQ ID NO: 1025 - DNA, 25 bp, Artificial
  Probe
gtaagtagttgtcatattctggaaa

SEQ ID NO: 1026 - DNA, 25 bp, Artificial
  Probe
ttagagttaagatatctcctctctt

SEQ ID NO: 1027 - DNA, 25 bp, Artificial
  Probe
atatctcctctctttggttagggaa

SEQ ID NO: 1028 - DNA, 25 bp, Artificial
  Probe
accattgtggaatgatgccctggct

SEQ ID NO: 1029 - DNA, 25 bp, Artificial
  Probe
agtattctacagctcaagactgcag

SEQ ID NO: 1030 - DNA, 25 bp, Artificial
  Probe
gactgcagtgcagatgacgacaact

SEQ ID NO: 1031 - DNA, 25 bp, Artificial
  Probe
gcagtgcagatgacgacaacttcct

SEQ ID NO: 1032 - DNA, 25 bp, Artificial
  Probe
gtgcagatgacgacaacttccttgt

SEQ ID NO: 1033 - DNA, 25 bp, Artificial
  Probe
cagatgacgacaacttccttgtgcc

SEQ ID NO: 1034 - DNA, 25 bp, Artificial
  Probe
cttccttgtgcccatagcggtggga

SEQ ID NO: 1035 - DNA, 25 bp, Artificial
  Probe
tgtgcccatagcggtgggagctgcc

SEQ ID NO: 1036 - DNA, 25 bp, Artificial
  Probe
gctgccttggcaggagtacttattc

SEQ ID NO: 1037 - DNA, 25 bp, Artificial
  Probe
ggcaggagtacttattctagtgttg

SEQ ID NO: 1038 - DNA, 25 bp, Artificial
  Probe
tctagtgttgctggcttattttatt

SEQ ID NO: 1039 - DNA, 25 bp, Artificial
  Probe
tattggtctcaagcaccatcatgct

SEQ ID NO: 1040 - DNA, 25 bp, Artificial
  Probe
caagagctacaatgtcacctccgtc

SEQ ID NO: 1041 - DNA, 25 bp, Artificial
  Probe
caggacttttgttccaggttgccag

SEQ ID NO: 1042 - DNA, 25 bp, Artificial
  Probe
cgagttcacgctgggcaacattaag

SEQ ID NO: 1043 - DNA, 25 bp, Artificial
  Probe
gttacctcgtccgagtggtgagcac

SEQ ID NO: 1044 - DNA, 25 bp, Artificial
  Probe
gggagtacttcaagatcaccctcta

SEQ ID NO: 1045 - DNA, 25 bp, Artificial
  Probe
agatcaccctctacgggagaaccaa

SEQ ID NO: 1046 - DNA, 25 bp, Artificial
  Probe
gggagaaccaaggagctgacttcgg

SEQ ID NO: 1047 - DNA, 25 bp, Artificial
  Probe
ctaaaggagaacttcatccgcttct

SEQ ID NO: 1048 - DNA, 25 bp, Artificial
  Probe
gtcccaatcgaccagtgtatcgacg

SEQ ID NO: 1049 - DNA, 25 bp, Artificial
  Probe
gacggctgagtgcacaggtgccgcc

SEQ ID NO: 1050 - DNA, 25 bp, Artificial
  Probe
caccagcccgaacaccattgaggga

SEQ ID NO: 1051 - DNA, 25 bp, Artificial
  Probe
cctccagtctggatcgtttgacggg

SEQ ID NO: 1052 - DNA, 25 bp, Artificial
  Probe
tttgacgggacttcaggttctttct

SEQ ID NO: 1053 - DNA, 25 bp, Artificial
  Probe
gaaatcgccgtgttactgttgcact

SEQ ID NO: 1054 - DNA, 25 bp, Artificial
  Probe
ctgttgcactgatgtccggagagac

SEQ ID NO: 1055 - DNA, 25 bp, Artificial
  Probe
gtcagactcccgcgtgaagatgtca

SEQ ID NO: 1056 - DNA, 25 bp, Artificial
  Probe
agggaaccgtgataagcctttctgg

SEQ ID NO: 1057 - DNA, 25 bp, Artificial
  Probe
gcctttctggtttcggagcacgtaa

SEQ ID NO: 1058 - DNA, 25 bp, Artificial
  Probe
cacgtaaatgcgtccctgtacagat

SEQ ID NO: 1059 - DNA, 25 bp, Artificial
  Probe
ttgttcagtgactattctcggggcc

SEQ ID NO: 1060 - DNA, 25 bp, Artificial
  Probe
cgaactggactgtgtgcaacgcttt

SEQ ID NO: 1061 - DNA, 25 bp, Artificial
  Probe
agaatgatgtccccgttgtatgtat

SEQ ID NO: 1062 - DNA, 25 bp, Artificial
  Probe
ggacaggggcctgctgaggaggtcc

SEQ ID NO: 1063 - DNA, 25 bp, Artificial
  Probe
gaaaacctctatgctgccgtgaagg

SEQ ID NO: 1064 - DNA, 25 bp, Artificial
  Probe
tggagctggacagtcagagcccaca

SEQ ID NO: 1065 - DNA, 25 bp, Artificial
  Probe
tccctcctcactgtctggggaattc

SEQ ID NO: 1066 - DNA, 25 bp, Artificial
  Probe
gatggacactgaggctgctgcatct

SEQ ID NO: 1067 - DNA, 25 bp, Artificial
  Probe
atctgaagcctcccaggatgtgacc

SEQ ID NO: 1068 - DNA, 25 bp, Artificial
  Probe
agacggaaggcaactgagcctcctc

SEQ ID NO: 1069 - DNA, 25 bp, Artificial
  Probe
ccaggaaggggaacctccagctgag

SEQ ID NO: 1070 - DNA, 25 bp, Artificial
  Probe
gaccccacactcagcagaaggagac

SEQ ID NO: 1071 - DNA, 25 bp, Artificial
  Probe
ggactgctgaaggcacgggagctgc

SEQ ID NO: 1072 - DNA, 25 bp, Artificial
  Probe
ggacccctaacacagaccatgagga

SEQ ID NO: 1073 - DNA, 25 bp, Artificial
  Probe
cgctgctgtgcctcgatggcaaacg

SEQ ID NO: 1074 - DNA, 25 bp, Artificial
  Probe
gaatcatgccgtggtgtctcggatg

SEQ ID NO: 1075 - DNA, 25 bp, Artificial
  Probe
agaaatggatctgactgcccggaca

SEQ ID NO: 1076 - DNA, 25 bp, Artificial
  Probe
gactgcccggacaagttttgcttat

SEQ ID NO: 1077 - DNA, 25 bp, Artificial
  Probe
caagttttgcttattccagtctgaa

SEQ ID NO: 1078 - DNA, 25 bp, Artificial
  Probe
aaaccttctgttcaatgacaacact

SEQ ID NO: 1079 - DNA, 25 bp, Artificial
  Probe
gtgtctggccagactccatggcaaa

SEQ ID NO: 1080 - DNA, 25 bp, Artificial
  Probe
gggaccacagtatgtcgcaggcatt

SEQ ID NO: 1081 - DNA, 25 bp, Artificial
  Probe
tcctggaagcctgtgaattcctcag

SEQ ID NO: 1082 - DNA, 25 bp, Artificial
  Probe
caagaaagcctcagccattcactgc

SEQ ID NO: 1083 - DNA, 25 bp, Artificial
  Probe
ttccctgctgtcgtcttagcaagaa

SEQ ID NO: 1084 - DNA, 25 bp, Artificial
  Probe
gacccgtccatttggcaggggtggc

SEQ ID NO: 1085 - DNA, 25 bp, Artificial
  Probe
caggggtggctgcctcatttagaga

SEQ ID NO: 1086 - DNA, 25 bp, Artificial
  Probe
cactggttgcacttatgatttcatg

SEQ ID NO: 1087 - DNA, 25 bp, Artificial
  Probe
tcatgtgcggggatcatctgccgtg

SEQ ID NO: 1088 - DNA, 25 bp, Artificial
  Probe
tgtactcttagatggattctccact

SEQ ID NO: 1089 - DNA, 25 bp, Artificial
  Probe
gattctccactcagttgcaacttgg

SEQ ID NO: 1090 - DNA, 25 bp, Artificial
  Probe
gcaacttggacttgtcctcagcagc

SEQ ID NO: 1091 - DNA, 25 bp, Artificial
  Probe
ctcagcagctggtaatcttgctctg

SEQ ID NO: 1092 - DNA, 25 bp, Artificial
  Probe
tcttgctctgcttgacaacatctga

SEQ ID NO: 1093 - DNA, 25 bp, Artificial
  Probe
aaaatgcacccaactagctctatgt

SEQ ID NO: 1094 - DNA, 25 bp, Artificial
  Probe
gagaccattgcaatgaatccccaat

SEQ ID NO: 1095 - DNA, 25 bp, Artificial
  Probe
ctcattgacttttctgctcagattg

SEQ ID NO: 1096 - DNA, 25 bp, Artificial
  Probe
gagcagctaatgttctggtctccga

SEQ ID NO: 1097 - DNA, 25 bp, Artificial
  Probe
gttccctattaagtggacggctcca

SEQ ID NO: 1098 - DNA, 25 bp, Artificial
  Probe
gtcctttggaatcctcctatacgaa

SEQ ID NO: 1099 - DNA, 25 bp, Artificial
  Probe
aactaatgccgacgtgatgaccgcc

SEQ ID NO: 1100 - DNA, 25 bp, Artificial
  Probe
ctgtcccagggctacaggatgcccc

SEQ ID NO: 1101 - DNA, 25 bp, Artificial
  Probe
caggatgccccgtgtggagaactgc

SEQ ID NO: 1102 - DNA, 25 bp, Artificial
  Probe
actgcccagatgagctctatgacat

SEQ ID NO: 1103 - DNA, 25 bp, Artificial
  Probe
gaccaacgtttgactacttacagag

SEQ ID NO: 1104 - DNA, 25 bp, Artificial
  Probe
gcgtcctggatgatttctacacagc

SEQ ID NO: 1105 - DNA, 25 bp, Artificial
  Probe
aagggcagtttgctggatttcctga

SEQ ID NO: 1106 - DNA, 25 bp, Artificial
  Probe
ttgcacggatctaagttattctccc

SEQ ID NO: 1107 - DNA, 25 bp, Artificial
  Probe
tattgcccggctcctagaatttatt

SEQ ID NO: 1108 - DNA, 25 bp, Artificial
  Probe
tttatttcctgacttacagcaagcg

SEQ ID NO: 1109 - DNA, 25 bp, Artificial
  Probe
ttatttcctgacttacagcaagcga

SEQ ID NO: 1110 - DNA, 25 bp, Artificial
  Probe
tatttcctgacttacagcaagcgag

SEQ ID NO: 1111 - DNA, 25 bp, Artificial
  Probe
tttcctgacttacagcaagcgagtt

SEQ ID NO: 1112 - DNA, 25 bp, Artificial
  Probe
tcctgacttacagcaagcgagttat

SEQ ID NO: 1113 - DNA, 25 bp, Artificial
  Probe
cctgacttacagcaagcgagttatc

SEQ ID NO: 1114 - DNA, 25 bp, Artificial
  Probe
ctgacttacagcaagcgagttatcg

SEQ ID NO: 1115 - DNA, 25 bp, Artificial
  Probe
tgacttacagcaagcgagttatcgt

SEQ ID NO: 1116 - DNA, 25 bp, Artificial
  Probe
ttacagcaagcgagttatcgtcttc

SEQ ID NO: 1117 - DNA, 25 bp, Artificial
  Probe
tacagcaagcgagttatcgtcttct

SEQ ID NO: 1118 - DNA, 25 bp, Artificial
  Probe
agcaagcgagttatcgtcttctgta

SEQ ID NO: 1119 - DNA, 25 bp, Artificial
  Probe
caagcgagttatcgtcttctgtatt

SEQ ID NO: 1120 - DNA, 25 bp, Artificial
  Probe
aagcgagttatcgtcttctgtattt

SEQ ID NO: 1121 - DNA, 25 bp, Artificial
  Probe
gcgagttatcgtcttctgtattttg

SEQ ID NO: 1122 - DNA, 25 bp, Artificial
  Probe
gagcaaggtaagtagaacatccata

SEQ ID NO: 1123 - DNA, 25 bp, Artificial
  Probe
gctgttttctttaggaaaatggctg

SEQ ID NO: 1124 - DNA, 25 bp, Artificial
  Probe
aatggctgttgatcttttctaagtg

SEQ ID NO: 1125 - DNA, 25 bp, Artificial
  Probe
tgatcttttctaagtgtgtttcact

SEQ ID NO: 1126 - DNA, 25 bp, Artificial
  Probe
ttcactttttcatgggatgatggct

SEQ ID NO: 1127 - DNA, 25 bp, Artificial
  Probe
atgatggctttgttgcagctgagat

SEQ ID NO: 1128 - DNA, 25 bp, Artificial
  Probe
ttgttgcagctgagattcatgtaac

SEQ ID NO: 1129 - DNA, 25 bp, Artificial
  Probe
tggtaataatagtttcacataggaa

SEQ ID NO: 1130 - DNA, 25 bp, Artificial
  Probe
agatgcaagttcactctgttagtta

SEQ ID NO: 1131 - DNA, 25 bp, Artificial
  Probe
cactctgttagttaactggtagtct

SEQ ID NO: 1132 - DNA, 25 bp, Artificial
  Probe
ggtagtctttgttaaggtgattcaa

SEQ ID NO: 1133 - DNA, 25 bp, Artificial
  Probe
ccccagtttttctctaagatataca

SEQ ID NO: 1134 - DNA, 25 bp, Artificial
  Probe
gatatacagtgcaatagctccccac

SEQ ID NO: 1135 - DNA, 25 bp, Artificial
  Probe
agttgacgccagccctgtaaagctg

SEQ ID NO: 1136 - DNA, 25 bp, Artificial
  Probe
gtcttcagtgaggtggctggggcga

SEQ ID NO: 1137 - DNA, 25 bp, Artificial
  Probe
ccaggccagagctctttcattgggg

SEQ ID NO: 1138 - DNA, 25 bp, Artificial
  Probe
gggcgagtgtggtgaggggacgtcc

SEQ ID NO: 1139 - DNA, 25 bp, Artificial
  Probe
acctgggggagtcaacactgggatg

SEQ ID NO: 1140 - DNA, 25 bp, Artificial
  Probe
aacactgggatggtctgtggggtgg

SEQ ID NO: 1141 - DNA, 25 bp, Artificial
  Probe
ggagggcctacggatgggtccgtag

SEQ ID NO: 1142 - DNA, 25 bp, Artificial
  Probe
tagcgacctttggaaaacgttagcg

SEQ ID NO: 1143 - DNA, 25 bp, Artificial
  Probe
acgttagcggtgtaacagtccagga

SEQ ID NO: 1144 - DNA, 25 bp, Artificial
  Probe
gaggtcccaaacagaattatcgaaa

SEQ ID NO: 1145 - DNA, 25 bp, Artificial
  Probe
aaaactcccaagatcagtcaacttt

SEQ ID NO: 1146 - DNA, 25 bp, Artificial
  Probe
agcaagcatctggaacaatggtgta

SEQ ID NO: 1147 - DNA, 25 bp, Artificial
  Probe
aatggtgtatgggttgtttatgtta

SEQ ID NO: 1148 - DNA, 25 bp, Artificial
  Probe
aggagataaacttcgactcttctgc

SEQ ID NO: 1149 - DNA, 25 bp, Artificial
  Probe
ctcttctgccttcaactgagaacag

SEQ ID NO: 1150 - DNA, 25 bp, Artificial
  Probe
ctgagaacagttgaccgcaagctga

SEQ ID NO: 1151 - DNA, 25 bp, Artificial
  Probe
ccgcaagctgaaactggtgtgtgga

SEQ ID NO: 1152 - DNA, 25 bp, Artificial
  Probe
ggtgtgtggaagtcacagcttcatc

SEQ ID NO: 1153 - DNA, 25 bp, Artificial
  Probe
aatgcaacaaacaacttccgcttaa

SEQ ID NO: 1154 - DNA, 25 bp, Artificial
  Probe
atagattagtttgctttctggaata

SEQ ID NO: 1155 - DNA, 25 bp, Artificial
  Probe
tccacctactccattgctttatgag

SEQ ID NO: 1156 - DNA, 25 bp, Artificial
  Probe
ggaaggcggtataatccctattcaa

SEQ ID NO: 1157 - DNA, 25 bp, Artificial
  Probe
aacttctgaccgcccagtaggaaga

SEQ ID NO: 1158 - DNA, 25 bp, Artificial
  Probe
gcttcttgactttaacatcagcatt

SEQ ID NO: 1159 - DNA, 25 bp, Artificial
  Probe
gagggttaaggctcagggattttat

SEQ ID NO: 1160 - DNA, 25 bp, Artificial
  Probe
tgaactgctggaactcacacatgcc

SEQ ID NO: 1161 - DNA, 25 bp, Artificial
  Probe
gcgagtctgagagcaagcccaaatg

SEQ ID NO: 1162 - DNA, 25 bp, Artificial
  Probe
gaatctgacacatctgggttcaaat

SEQ ID NO: 1163 - DNA, 25 bp, Artificial
  Probe
gaaactgtcacttattacctgtatg

SEQ ID NO: 1164 - DNA, 25 bp, Artificial
  Probe
taatctctctgatctatttttcctc

SEQ ID NO: 1165 - DNA, 25 bp, Artificial
  Probe
acaactactttgtcggttgctctga

SEQ ID NO: 1166 - DNA, 25 bp, Artificial
  Probe
gatactagccttaacatgtacctgt

SEQ ID NO: 1167 - DNA, 25 bp, Artificial
  Probe
tagccttaacatgtacctgtcaatg

SEQ ID NO: 1168 - DNA, 25 bp, Artificial
  Probe
atgtacctgtcaatgttatggatat

SEQ ID NO: 1169 - DNA, 25 bp, Artificial
  Probe
aatgattgaaacccatgcatggtgt

SEQ ID NO: 1170 - DNA, 25 bp, Artificial
  Probe
atgcatggtgttagacaatttttct

SEQ ID NO: 1171 - DNA, 25 bp, Artificial
  Probe
gtgattagtgattatcagagcaaac

SEQ ID NO: 1172 - DNA, 25 bp, Artificial
  Probe
gagcaaacatcatgtagatagcaca

SEQ ID NO: 1173 - DNA, 25 bp, Artificial
  Probe
gcaaacatcatgtagatagcacaag

SEQ ID NO: 1174 - DNA, 25 bp, Artificial
  Probe
atttcaataccttttagatttcata

SEQ ID NO: 1175 - DNA, 25 bp, Artificial
  Probe
aagtgcagtgtatataatgcctact

SEQ ID NO: 1176 - DNA, 25 bp, Artificial
  Probe
tataatgcctactgaaagactgtaa

SEQ ID NO: 1177 - DNA, 25 bp, Artificial
  Probe
gaaaagccgttcaccaaatcgacca

SEQ ID NO: 1178 - DNA, 25 bp, Artificial
  Probe
tcgaccagcttcagcgagagcagcg

SEQ ID NO: 1179 - DNA, 25 bp, Artificial
  Probe
gatccggatggacagcatcggctcc

SEQ ID NO: 1180 - DNA, 25 bp, Artificial
  Probe
cgctccgactccgacagggaagaaa

SEQ ID NO: 1181 - DNA, 25 bp, Artificial
  Probe
gagcacggactatctcacaggtgat

SEQ ID NO: 1182 - DNA, 25 bp, Artificial
  Probe
ggtgatctggactggagcagcagca

SEQ ID NO: 1183 - DNA, 25 bp, Artificial
  Probe
gcagtgtgagcgactctgacgagcg

SEQ ID NO: 1184 - DNA, 25 bp, Artificial
  Probe
agcatgcagagcctcggcagtgatg

SEQ ID NO: 1185 - DNA, 25 bp, Artificial
  Probe
agtgatgagggctattccagcacca

SEQ ID NO: 1186 - DNA, 25 bp, Artificial
  Probe
gtcacaaggcgtgtcttggtctcta

SEQ ID NO: 1187 - DNA, 25 bp, Artificial
  Probe
aactcccttgcacgtaaacttcagt

SEQ ID NO: 1188 - DNA, 25 bp, Artificial
  Probe
gcatcctcaaacatggcgccaagga

SEQ ID NO: 1189 - DNA, 25 bp, Artificial
  Probe
cctcaaacatggcgccaaggataaa

SEQ ID NO: 1190 - DNA, 25 bp, Artificial
  Probe
tcccagttcccagtggaagaaacag

SEQ ID NO: 1191 - DNA, 25 bp, Artificial
  Probe
agtgcgtgccgagctgaggcagatg

SEQ ID NO: 1192 - DNA, 25 bp, Artificial
  Probe
gcgtgccgagctgaggcagatgttc

SEQ ID NO: 1193 - DNA, 25 bp, Artificial
  Probe
tgccgagctgaggcagatgttccca

SEQ ID NO: 1194 - DNA, 25 bp, Artificial
  Probe
ccccagagccctgggctatagtctc

SEQ ID NO: 1195 - DNA, 25 bp, Artificial
  Probe
cagagccctgggctatagtctctga

SEQ ID NO: 1196 - DNA, 25 bp, Artificial
  Probe
cccacacaaatgcaagctcaccaag

SEQ ID NO: 1197 - DNA, 25 bp, Artificial
  Probe
acacaaatgcaagctcaccaaggtc

SEQ ID NO: 1198 - DNA, 25 bp, Artificial
  Probe
acaaatgcaagctcaccaaggtccc

SEQ ID NO: 1199 - DNA, 25 bp, Artificial
  Probe
gttcggctgctgtcggatgaggacg

SEQ ID NO: 1200 - DNA, 25 bp, Artificial
  Probe
tacagggtctacaacacgatgccat

SEQ ID NO: 1201 - DNA, 25 bp, Artificial
  Probe
agcagaggctctatttgacttcact

SEQ ID NO: 1202 - DNA, 25 bp, Artificial
  Probe
gctggagatgtgatcttcctcctca

SEQ ID NO: 1203 - DNA, 25 bp, Artificial
  Probe
cttcctcctcagtcggatcaacaaa

SEQ ID NO: 1204 - DNA, 25 bp, Artificial
  Probe
ctctctccttcgtgaagatcctcaa

SEQ ID NO: 1205 - DNA, 25 bp, Artificial
  Probe
tggctgcgttgctactactacgaag

SEQ ID NO: 1206 - DNA, 25 bp, Artificial
  Probe
tactacgaagacaccatcagcacca

SEQ ID NO: 1207 - DNA, 25 bp, Artificial
  Probe
atcagcaccatcaaggacatcgcgg

SEQ ID NO: 1208 - DNA, 25 bp, Artificial
  Probe
cactcccctattgaaagacctgctg

SEQ ID NO: 1209 - DNA, 25 bp, Artificial
  Probe
ggagctcacaaggcgggagttccag

SEQ ID NO: 1210 - DNA, 25 bp, Artificial
  Probe
tggaggaagatctcagcagcactcc

SEQ ID NO: 1211 - DNA, 25 bp, Artificial
  Probe
cactcccctattgaaagacctgctg

SEQ ID NO: 1212 - DNA, 25 bp, Artificial
  Probe
ggagctcacaaggcgggagttccag

SEQ ID NO: 1213 - DNA, 25 bp, Artificial
  Probe
ggacatagctctgaattaccgggac

SEQ ID NO: 1214 - DNA, 25 bp, Artificial
  Probe
gctctgaattaccgggacgctgagg

SEQ ID NO: 1215 - DNA, 25 bp, Artificial
  Probe
gttcggctgctgtcggatgaggacg

SEQ ID NO: 1216 - DNA, 25 bp, Artificial
  Probe
gatgaggacgtagcgctcatggtgc

SEQ ID NO: 1217 - DNA, 25 bp, Artificial
  Probe
ggaagctgcacatcacgcagaagga

SEQ ID NO: 1218 - DNA, 25 bp, Artificial
  Probe
tcgtgaagatcctcaaagacttccc

SEQ ID NO: 1219 - DNA, 25 bp, Artificial
  Probe
tggctgcgttgctactactacgaag

SEQ ID NO: 1220 - DNA, 25 bp, Artificial
  Probe
tgctactactacgaagacaccatca

SEQ ID NO: 1221 - DNA, 25 bp, Artificial
  Probe
ttttatttttacacccatcagattt

SEQ ID NO: 1222 - DNA, 25 bp, Artificial
  Probe
ttattacaactatgagagcctccca

SEQ ID NO: 1223 - DNA, 25 bp, Artificial
  Probe
cctcccaagtcatcttatcaactca

SEQ ID NO: 1224 - DNA, 25 bp, Artificial
  Probe
ggatgaccacactagcacagaagag

SEQ ID NO: 1225 - DNA, 25 bp, Artificial
  Probe
ttaaagcaggtgattcctcccttgg

SEQ ID NO: 1226 - DNA, 25 bp, Artificial
  Probe
gattcctcccttggcgggagagctc

SEQ ID NO: 1227 - DNA, 25 bp, Artificial
  Probe
gagctctctcagtgtgaacatgcct

SEQ ID NO: 1228 - DNA, 25 bp, Artificial
  Probe
catgccttctgtgggcggaaatcag

SEQ ID NO: 1229 - DNA, 25 bp, Artificial
  Probe
ggaaatcaggaagccaccagctgtt

SEQ ID NO: 1230 - DNA, 25 bp, Artificial
  Probe
ggagagtgccttgcttttatttcag

SEQ ID NO: 1231 - DNA, 25 bp, Artificial
  Probe
tgctcctctaacagcattgctcttt

SEQ ID NO: 1232 - DNA, 25 bp, Artifici
agatctaatattgactgcctctgcc

SEQ ID NO: 1233 - DNA, 25 bp, Artificial
  Probe
cctctgcctgtcgcatgagaacatt

SEQ ID NO: 1234 - DNA, 25 bp, Artificial
  Probe
caattgtattacttcctctgttcgc

SEQ ID NO: 1235 - DNA, 25 bp, Artificial
  Probe
ttcgcgactagttggctctgagata

SEQ ID NO: 1236 - DNA, 25 bp, Artificial
  Probe
gtgaggctccggatgtttctggaat

SEQ ID NO: 1237 - DNA, 25 bp, Artificial
  Probe
ataaaggcatttcaaagtctcactt

SEQ ID NO: 1238 - DNA, 25 bp, Artificial
  Probe
tctactgaacagtccatcttcttta

SEQ ID NO: 1239 - DNA, 25 bp, Artificial
  Probe
tcttctttatacaatgaccacatcc

SEQ ID NO: 1240 - DNA, 25 bp, Artificial
  Probe
gaccacatcctgaaaagggtgttgc

SEQ ID NO: 1241 - DNA, 25 bp, Artificial
  Probe
agggtgttgctaagctgtaaccgat

SEQ ID NO: 1242 - DNA, 25 bp, Artificial
  Probe
agctgtaaccgatatgcacttgaaa

SEQ ID NO: 1243 - DNA, 25 bp, Artificial
  Probe
atttaagttgtgattacctgctgca

SEQ ID NO: 1244 - DNA, 25 bp, Artificial
  Probe
aagtggcatgggggaccctgtgcat

SEQ ID NO: 1245 - DNA, 25 bp, Artificial
  Probe
gaccctgtgcatctgtgcatttggc

SEQ ID NO: 1246 - DNA, 25 bp, Artificial
  Probe
tccatttctggacatgacgtctgtg

SEQ ID NO: 1247 - DNA, 25 bp, Artificial
  Probe
gacgtctgtggtttaagctttgtga

SEQ ID NO: 1248 - DNA, 25 bp, Artificial
  Probe
aatgtgctttgattcgaagggtctt

SEQ ID NO: 1249 - DNA, 25 bp, Artificial
  Probe
taatcgtcaaccacttttaaacata

SEQ ID NO: 1250 - DNA, 25 bp, Artificial
  Probe
agaattcacacaactactttcatga

SEQ ID NO: 1251 - DNA, 25 bp, Artificial
  Probe
attccaagagtatcccagtattagc

SEQ ID NO: 1252 - DNA, 25 bp, Artificial
  Probe
atataggcacattaccattcatagt

SEQ ID NO: 1253 - DNA, 25 bp, Artificial
  Probe
aatttgatgcgatctgctcagtaat

SEQ ID NO: 1254 - DNA, 25 bp, Artificial
  Probe
cagcctgcaccgcgaggtggagaag

SEQ ID NO: 1255 - DNA, 25 bp, Artificial
  Probe
gaccaggagctcgacttcatcctgt

SEQ ID NO: 1256 - DNA, 25 bp, Artificial
  Probe
tctacctgcagcacgcggatgagga

SEQ ID NO: 1257 - DNA, 25 bp, Artificial
  Probe
gctggctgagaacatcgacgcacag

SEQ ID NO: 1258 - DNA, 25 bp, Artificial
  Probe
tcgacgcacagctcaagcgcatggc

SEQ ID NO: 1259 - DNA, 25 bp, Artificial
  Probe
tggcccaggatctcaaggacatcat

SEQ ID NO: 1260 - DNA, 25 bp, Artificial
  Probe
aaggacatcatcgagcacctgaaca

SEQ ID NO: 1261 - DNA, 25 bp, Artificial
  Probe
gcagtggatcgaccagaactcggcc

SEQ ID NO: 1262 - DNA, 25 bp, Artificial
  Probe
gatcacctttgactgagcgacagca

SEQ ID NO: 1263 - DNA, 25 bp, Artificial
  Probe
atgaggggaatgcgccctgttgtct

SEQ ID NO: 1264 - DNA, 25 bp, Artificial
  Probe
gttgtctgtagtttggggttgtggc

SEQ ID NO: 1265 - DNA, 25 bp, Artificial
  Probe
agttctctctgatagtgagcgagac

SEQ ID NO: 1266 - DNA, 25 bp, Artificial
  Probe
gaattacagctgatacctgatcaac

SEQ ID NO: 1267 - DNA, 25 bp, Artificial
  Probe
gatacctgatcaacttcgacatttg

SEQ ID NO: 1268 - DNA, 25 bp, Artificial
  Probe
acttcgacatttgggcaatgccatc

SEQ ID NO: 1269 - DNA, 25 bp, Artificial
  Probe
gggcaatgccatcaaacaggttact

SEQ ID NO: 1270 - DNA, 25 bp, Artificial
  Probe
atgccatcaaacaggttactatgaa

SEQ ID NO: 1271 - DNA, 25 bp, Artificial
  Probe
gagaaggtgttgagtcttccaaaac

SEQ ID NO: 1272 - DNA, 25 bp, Artificial
  Probe
tctcagtgcctaccagcgaaagtgc

SEQ ID NO: 1273 - DNA, 25 bp, Artificial
  Probe
gtgcctaccagcgaaagtgcattca

SEQ ID NO: 1274 - DNA, 25 bp, Artificial
  Probe
agcgaaagtgcattcagtccatcct

SEQ ID NO: 1275 - DNA, 25 bp, Artificial
  Probe
gaaagtgcattcagtccatcctgaa

SEQ ID NO: 1276 - DNA, 25 bp, Artificial
  Probe
ctccagccccagagctgaaaacacc

SEQ ID NO: 1277 - DNA, 25 bp, Artificial
  Probe
cctatttgagggtgtctgtctggag

SEQ ID NO: 1278 - DNA, 25 bp, Artificial
  Probe
ttagggggaagtgagcgcctcccat

SEQ ID NO: 1279 - DNA, 25 bp, Artificial
  Probe
taaggctttccccaatgatgtcggt

SEQ ID NO: 1280 - DNA, 25 bp, Artificial
  Probe
gatgtcggtaatttctgatgtttct

SEQ ID NO: 1281 - DNA, 25 bp, Artificial
  Probe
tctgaagttcccaggactcacacac

SEQ ID NO: 1282 - DNA, 25 bp, Artificial
  Probe
cacccagtgtgacaaccctcggtgt

SEQ ID NO: 1283 - DNA, 25 bp, Artificial
  Probe
gacaaccctcggtgtggatataccc

SEQ ID NO: 1284 - DNA, 25 bp, Artificial
  Probe
ccacccccactttctataaatgtag

SEQ ID NO: 1285 - DNA, 25 bp, Artificial
  Probe
ggcctagaatacgcttctctgttgc

SEQ ID NO: 1286 - DNA, 25 bp, Artificial
  Probe
gggcaggggatgtcgtgaagatggc

SEQ ID NO: 1287 - DNA, 25 bp, Artificial
  Probe
gacatagcacgaatctgttaccagt

SEQ ID NO: 1288 - DNA, 25 bp, Artificial
  Probe
ggaggatgagccacagaaattgcat

SEQ ID NO: 1289 - DNA, 25 bp, Artificial
  Probe
taatttcaagtcttcctgatacatg

SEQ ID NO: 1290 - DNA, 25 bp, Artificial
  Probe
gaatagtgtggttcagtgagctgca

SEQ ID NO: 1291 - DNA, 25 bp, Artificial
  Probe
ctgcactgacctctacattttgtat

SEQ ID NO: 1292 - DNA, 25 bp, Artificial
  Probe
ctatgttcagaacttcatctgccac

SEQ ID NO: 1293 - DNA, 25 bp, Artificial
  Probe
agtacaaatctgtgctacactggat

SEQ ID NO: 1294 - DNA, 25 bp, Artificial
  Probe
atttatgaattttacttgcacctta

SEQ ID NO: 1295 - DNA, 25 bp, Artificial
  Probe
ttacttgcaccttatagttcatagc

SEQ ID NO: 1296 - DNA, 25 bp, Artificial
  Probe
tataccaatgacttccatattttaa

SEQ ID NO: 1297 - DNA, 25 bp, Artificial
  Probe
caactttatgttgcaggaaaccctt

SEQ ID NO: 1298 - DNA, 25 bp, Artificial
  Probe
gagtattactgcacagccttcaaca

SEQ ID NO: 1299 - DNA, 25 bp, Artificial
  Probe
tcattgctctcttgatcattgcggc

SEQ ID NO: 1300 - DNA, 25 bp, Artificial
  Probe
tggaaatgtccaggccagcagtacc

SEQ ID NO: 1301 - DNA, 25 bp, Artificial
  Probe
taccacttctgaactccaacaacga

SEQ ID NO: 1302 - DNA, 25 bp, Artificial
  Probe
ggaagctaacagtcattacggtcac

SEQ ID NO: 1303 - DNA, 25 bp, Artificial
  Probe
gagcctctgaactcagacgtgcagt

SEQ ID NO: 1304 - DNA, 25 bp, Artificial
  Probe
gtgtcctcagctgagtctcacaaag

SEQ ID NO: 1305 - DNA, 25 bp, Artificial
  Probe
agtgaagtccggaaagctgtccctg

SEQ ID NO: 1306 - DNA, 25 bp, Artificial
  Probe
ctgtccctgatgccgtggaaagcag

SEQ ID NO: 1307 - DNA, 25 bp, Artificial
  Probe
gaaggctcccttgatggaacttaga

SEQ ID NO: 1308 - DNA, 25 bp, Artificial
  Probe
gacagcaaggccagatgcacatccc

SEQ ID NO: 1309 - DNA, 25 bp, Artificial
  Probe
aaggcggctgcagaagatgtcaatg

SEQ ID NO: 1310 - DNA, 25 bp, Artificial
  Probe
agatgtcaatgttactttcgaagat

SEQ ID NO: 1311 - DNA, 25 bp, Artificial
  Probe
aaacctagaagatgcttgtgatgac

SEQ ID NO: 1312 - DNA, 25 bp, Artificial
  Probe
ttgtgatgacatcatgcttgcagat

SEQ ID NO: 1313 - DNA, 25 bp, Artificial
  Probe
gatgattgcttaatgataccttatc

SEQ ID NO: 1314 - DNA, 25 bp, Artificial
  Probe
gataccttatcaaattggtgatgtc

SEQ ID NO: 1315 - DNA, 25 bp, Artificial
  Probe
ggtgatgtcttcattagccattctc

SEQ ID NO: 1316 - DNA, 25 bp, Artificial
  Probe
ttgacgccttagaatccagagtgga

SEQ ID NO: 1317 - DNA, 25 bp, Artificial
  Probe
ggaatcaattcagcgagtgttagca

SEQ ID NO: 1318 - DNA, 25 bp, Artificial
  Probe
gttcagttgtatgcaaaattcggga

SEQ ID NO: 1319 - DNA, 25 bp, Artificial
  Probe
gggagcaacataaaccttgaagctg

SEQ ID NO: 1320 - DNA, 25 bp, Artificial
  Probe
atgatgtttaccagtccatttcagt

SEQ ID NO: 1321 - DNA, 25 bp, Artificial
  Probe
tttaccagtccatttcagttcttca

SEQ ID NO: 1322 - DNA, 25 bp, Artificial
  Probe
ctcttatagccccacataggttaga

SEQ ID NO: 1323 - DNA, 25 bp, Artificial
  Probe
actacttttgctgtataccaagcta

SEQ ID NO: 1324 - DNA, 25 bp, Artificial
  Probe
gtaagagacagagtgcattcatttg

SEQ ID NO: 1325 - DNA, 25 bp, Artificial
  Probe
tgcattcatttgcacccagggttgg

SEQ ID NO: 1326 - DNA, 25 bp, Artificial
  Probe
atattggagatacttggctatttgt

SEQ ID NO: 1327 - DNA, 25 bp, Artificial
  Probe
acctcacctgcccatgaaggctaaa

SEQ ID NO: 1328 - DNA, 25 bp, Artificial
  Probe
tggatggttaaacacctgtctctgt

SEQ ID NO: 1329 - DNA, 25 bp, Artificial
  Probe
gatgcttaagaaattacctcacata

SEQ ID NO: 1330 - DNA, 25 bp, Artificial
  Probe
attacctcacataaacattttacca

SEQ ID NO: 1331 - DNA, 25 bp, Artificial
  Probe
tggacttctcaaaccaacagtggcc

SEQ ID NO: 1332 - DNA, 25 bp, Artificial
  Probe
cttctcaaaccaacagtggcctctc

SEQ ID NO: 1333 - DNA, 25 bp, Artificial
  Probe
ctcaaaccaacagtggcctctcaga

SEQ ID NO: 1334 - DNA, 25 bp, Artificial
  Probe
aacagtggcctctcagaaccagaac

SEQ ID NO: 1335 - DNA, 25 bp, Artificial
  Probe
tcccacctagcaagttagtatctga

SEQ ID NO: 1336 - DNA, 25 bp, Artificial
  Probe
gtatctgatgacttggattcatctt

SEQ ID NO: 1337 - DNA, 25 bp, Artificial
  Probe
gacttggattcatctttagccaacc

SEQ ID NO: 1338 - DNA, 25 bp, Artificial
  Probe
gattcatctttagccaaccttgtgg

SEQ ID NO: 1339 - DNA, 25 bp, Artificial
  Probe
tcatctttagccaaccttgtgggca

SEQ ID NO: 1340 - DNA, 25 bp, Artificial
  Probe
ttagccaaccttgtgggcaatcttg

SEQ ID NO: 1341 - DNA, 25 bp, Artificial
  Probe
aaccttgtgggcaatcttggcatcg

SEQ ID NO: 1342 - DNA, 25 bp, Artificial
  Probe
aaatggactgatctttaaactattc

SEQ ID NO: 1343 - DNA, 25 bp, Artificial
  Probe
aactattcagtcttactgggatttt

SEQ ID NO: 1344 - DNA, 25 bp, Artificial
  Probe
ataaacagtgccagtattcatagga

SEQ ID NO: 1345 - DNA, 25 bp, Artificial
  Probe
gtgagaaactgtaatatttggccat

SEQ ID NO: 1346 - DNA, 25 bp, Artificial
  Probe
gccattattctattcaacaggtttt

SEQ ID NO: 1347 - DNA, 25 bp, Artificial
  Probe
tcaacaggttttagaggcatgccac

SEQ ID NO: 1348 - DNA, 25 bp, Artificial
  Probe
agttgcctttgcctgtaaaacatgt

SEQ ID NO: 1349 - DNA, 25 bp, Artificial
  Probe
tttcaacctttctggataccttaat

SEQ ID NO: 1350 - DNA, 25 bp, Artificial
  Probe
gtaactgtcagtttgcactggtcgg

SEQ ID NO: 1351 - DNA, 25 bp, Artificial
  Probe
tgcactggtcggtatatggaaacac

SEQ ID NO: 1352 - DNA, 25 bp, Artificial
  Probe
tatggaaacacattgctctaccctg

SEQ ID NO: 1353 - DNA, 25 bp, Artificial
  Probe
atgcctattctggtgttgcgtttgt

SEQ ID NO: 1354 - DNA, 25 bp, Artificial
  Probe
gacgttatcctctcagattcttatc

SEQ ID NO: 1355 - DNA, 25 bp, Artificial
  Probe
tatatcagtgcacaggcgcatccca

SEQ ID NO: 1356 - DNA, 25 bp, Artificial
  Probe
gcatcccaggcctgtacagatgtat

SEQ ID NO: 1357 - DNA, 25 bp, Artificial
  Probe
taccaggttttacacttgcatctct

SEQ ID NO: 1358 - DNA, 25 bp, Artificial
  Probe
aattggcctcttcctaagtatatta

SEQ ID NO: 1359 - DNA, 25 bp, Artificial
  Probe
tttatccttacattttatgcctccc

SEQ ID NO: 1360 - DNA, 25 bp, Artificial
  Probe
tatgcctccccctaaattaatgact

SEQ ID NO: 1361 - DNA, 25 bp, Artificial
  Probe
agcggctaggttttattcatactgt

SEQ ID NO: 1362 - DNA, 25 bp, Artificial
  Probe
tgaatttgtgccactttaatccttc

SEQ ID NO: 1363 - DNA, 25 bp, Artificial
  Probe
ttccactatcattcccattttgtta

SEQ ID NO: 1364 - DNA, 25 bp, Artificial
  Probe
tgctgtgctacaacgtcggtcagaa

SEQ ID NO: 1365 - DNA, 25 bp, Artificial
  Probe
actactgatgaatcgtcctcgtgct

SEQ ID NO: 1366 - DNA, 25 bp, Artificial
  Probe
tgaagtgcgttaagctggaccgacc

SEQ ID NO: 1367 - DNA, 25 bp, Artificial
  Probe
tggaccgacctagatttgaacgtgt

SEQ ID NO: 1368 - DNA, 25 bp, Artificial
  Probe
atttgaacgtgttcttggcccatgc

SEQ ID NO: 1369 - DNA, 25 bp, Artificial
  Probe
ccaatccatgcttcactcatgcaaa

SEQ ID NO: 1370 - DNA, 25 bp, Artificial
  Probe
gcttcactcatgcaaactgctttat

SEQ ID NO: 1371 - DNA, 25 bp, Artificial
  Probe
attgcaccattttcaatttggagca

SEQ ID NO: 1372 - DNA, 25 bp, Artificial
  Probe
agtgggaacgtcttacggtagctga

SEQ ID NO: 1373 - DNA, 25 bp, Artificial
  Probe
gtagctgatgcattggaaccagtgc

SEQ ID NO: 1374 - DNA, 25 bp, Artificial
  Probe
attttagaggggtcagctgctgtgc

SEQ ID NO: 1375 - DNA, 25 bp, Artificial
  Probe
tttgataacctgtcttccttgtttc

SEQ ID NO: 1376 - DNA, 25 bp, Artificial
  Probe
gtcaattagtagcttaccacagata

SEQ ID NO: 1377 - DNA, 25 bp, Artificial
  Probe
cacagatactgtttcctaccattta

SEQ ID NO: 1378 - DNA, 25 bp, Artificial
  Probe
tgatttttgcattaagtggtctaga

SEQ ID NO: 1379 - DNA, 25 bp, Artificial
  Probe
gtggtctagaattcttttgcaatgc

SEQ ID NO: 1380 - DNA, 25 bp, Artificial
  Probe
tttgcaacagaattttgtagcctta

SEQ ID NO: 1381 - DNA, 25 bp, Artificial
  Probe
gaaaaacctgactgcaaatcatgtc

SEQ ID NO: 1382 - DNA, 25 bp, Artificial
  Probe
cacataagggctggttatttacctc

SEQ ID NO: 1383 - DNA, 25 bp, Artificial
  Probe
aggacttttaacctttgctgacaag

SEQ ID NO: 1384 - DNA, 25 bp, Artificial
  Probe
ttgtctgtttcagttatacttgtga

SEQ ID NO: 1385 - DNA, 25 bp, Artificial
  Probe
aatactttgccttggaatagattat

SEQ ID NO: 1386 - DNA, 25 bp, Artificial
  Probe
tgatcccatcaacactattcttgta

SEQ ID NO: 1387 - DNA, 25 bp, Artificial
  Probe
ctatttttttctcatacgattacta

SEQ ID NO: 1388 - DNA, 25 bp, Artificial
  Probe
acgattactatagtccagtttacca

SEQ ID NO: 1389 - DNA, 25 bp, Artificial
  Probe
tcttgagatgattgcttaccttaaa

SEQ ID NO: 1390 - DNA, 25 bp, Artificial
  Probe
gttctacttactgtattaactggca

SEQ ID NO: 1391 - DNA, 25 bp, Artificial
  Probe
aagatctgaattgctgtgtatgtta

SEQ ID NO: 1392 - DNA, 25 bp, Artificial
  Probe
gttacgctgtattcagaaccagttt

SEQ ID NO: 1393 - DNA, 25 bp, Artificial
  Probe
gaaccagtttctaaccagcctgtga

SEQ ID NO: 1394 - DNA, 25 bp, Artificial
  Probe
agtggtgttgacattctggatcttc

SEQ ID NO: 1395 - DNA, 25 bp, Artificial
  Probe
gtgacgtcacttacctgtctaacgt

SEQ ID NO: 1396 - DNA, 25 bp, Artificial
  Probe
tacctgtctaacgtggtgtgggaga

SEQ ID NO: 1397 - DNA, 25 bp, Artificial
  Probe
gagaacttcagagcactatgcactg

SEQ ID NO: 1398 - DNA, 25 bp, Artificial
  Probe
tcaagaattccatttttcacagagt

SEQ ID NO: 1399 - DNA, 25 bp, Artificial
  Probe
gtaattccagattttgtttgccaag

SEQ ID NO: 1400 - DNA, 25 bp, Artificial
  Probe
ggaacaggcggacagtccatttatg

SEQ ID NO: 1401 - DNA, 25 bp, Artificial
  Probe
gatgtgaaacatactggtcctggtt

SEQ ID NO: 1402 - DNA, 25 bp, Artificial
  Probe
gccaatcaaggccagaataccaata

SEQ ID NO: 1403 - DNA, 25 bp, Artificial
  Probe
aagcatgtagtatttgggtttgtta

SEQ ID NO: 1404 - DNA, 25 bp, Artificial
  Probe
agattgaatcatttggttctcccaa

SEQ ID NO: 1405 - DNA, 25 bp, Artificial
  Probe
gcagttggattgaagcttagctatt

SEQ ID NO: 1406 - DNA, 25 bp, Artificial
  Probe
tggacgtttccgatttacaaatgta

SEQ ID NO: 1407 - DNA, 25 bp, Artificial
  Probe
gcagcttatagctgttgtcactttt

SEQ ID NO: 1408 - DNA, 25 bp, Artificial
  Probe
cttgttttttcccagagcaaccatg

SEQ ID NO: 1409 - DNA, 25 bp, Artificial
  Probe
ggatgacctggtgtcccattataaa

SEQ ID NO: 1410 - DNA, 25 bp, Artificial
  Probe
gcagtaggcttatgtacacctctta

SEQ ID NO: 1411 - DNA, 25 bp, Artificial
  Probe
tgataggactgcttgggtcctccac

SEQ ID NO: 1412 - DNA, 25 bp, Artificial
  Probe
cactgtcctctgtcaatctagttag

SEQ ID NO: 1413 - DNA, 25 bp, Artificial
  Probe
gttagacgtgcttctgaatgactgt

SEQ ID NO: 1414 - DNA, 25 bp, Artificial
  Probe
ggaactagaaactacacctggcttg

SEQ ID NO: 1415 - DNA, 25 bp, Artificial
  Probe
cacctggcttggagtcagatttagt

SEQ ID NO: 1416 - DNA, 25 bp, Artificial
  Probe
gcagtagtactaaggcgtcttttgt

SEQ ID NO: 1417 - DNA, 25 bp, Artificial
  Probe
agaatttatcctaatggcctttata

SEQ ID NO: 1418 - DNA, 25 bp, Artificial
  Probe
agtcaccacctagaactgggtattc

SEQ ID NO: 1419 - DNA, 25 bp, Artificial
  Probe
gtttacctgggctcaatggtttgaa

SEQ ID NO: 1420 - DNA, 25 bp, Artificial
  Probe
gaaacccagcacatcaatatgacct

SEQ ID NO: 1421 - DNA, 25 bp, Artificial
  Probe
tcaatatgacctcccagcaatgcac

SEQ ID NO: 1422 - DNA, 25 bp, Artificial
  Probe
ttccttcttacaacttttgctaacg

SEQ ID NO: 1423 - DNA, 25 bp, Artificial
  Probe
gtaacccaaatatgacctgtcctag

SEQ ID NO: 1424 - DNA, 25 bp, Artificial
  Probe
gacctgtcctagtaacaaaactcgc

SEQ ID NO: 1425 - DNA, 25 bp, Artificial
  Probe
aactcgcaaaaattgtcaccacagt

SEQ ID NO: 1426 - DNA, 25 bp, Artificial
  Probe
aagccaggtgcctttaatccactgt

SEQ ID NO: 1427 - DNA, 25 bp, Artificial
  Probe
cagagatcaacgacgagaccctcca

SEQ ID NO: 1428 - DNA, 25 bp, Artificial
  Probe
cagttcacctggatagaatcatcta

SEQ ID NO: 1429 - DNA, 25 bp, Artificial
  Probe
atcccatctctccatatactttggg

SEQ ID NO: 1430 - DNA, 25 bp, Artificial
  Probe
gttgttccttctcaaggcgattcag

SEQ ID NO: 1431 - DNA, 25 bp, Artificial
  Probe
ggcgattcagactctgacacagaca

SEQ ID NO: 1432 - DNA, 25 bp, Artificial
  Probe
gtgacagaacatacccctttactga

SEQ ID NO: 1433 - DNA, 25 bp, Artificial
  Probe
tgagacctttagcttctgtcagtgc

SEQ ID NO: 1434 - DNA, 25 bp, Artificial
  Probe
ccagtcatttggggctttatcggaa

SEQ ID NO: 1435 - DNA, 25 bp, Artificial
  Probe
ttatcggaatcccgctcacatcaga

SEQ ID NO: 1436 - DNA, 25 bp, Artificial
  Probe
gaacatgatgtcgtggtccagttgc

SEQ ID NO: 1437 - DNA, 25 bp, Artificial
  Probe
gtggtccagttgcagcctaatggtg

SEQ ID NO: 1438 - DNA, 25 bp, Artificial
  Probe
gattttttgctcccttcaaagattt

SEQ ID NO: 1439 - DNA, 25 bp, Artificial
  Probe
gactggtgctgtaactcaagcatca

SEQ ID NO: 1440 - DNA, 25 bp, Artificial
  Probe
aaacctgtccagtgtgcaagcaaaa

SEQ ID NO: 1441 - DNA, 25 bp, Artificial
  Probe
gtgcaaacactgctagagtcatttt

SEQ ID NO: 1442 - DNA, 25 bp, Artificial
  Probe
gctagagtcattttgaagctcaagc

SEQ ID NO: 1443 - DNA, 25 bp, Artificial
  Probe
ttcactttgtttcttacatgtgtac

SEQ ID NO: 1444 - DNA, 25 bp, Artificial
  Probe
gaaaatggccatctttaagcatatt

SEQ ID NO: 1445 - DNA, 25 bp, Artificial
  Probe
tttctgccactttatttaaaggcaa

SEQ ID NO: 1446 - DNA, 25 bp, Artificial
  Probe
ttcctcttttccagggctttgtatg

SEQ ID NO: 1447 - DNA, 25 bp, Artificial
  Probe
ccagggctttgtatgcacttgtata

SEQ ID NO: 1448 - DNA, 25 bp, Artificial
  Probe
gtagagtttgaatttcagtctgtaa

SEQ ID NO: 1449 - DNA, 25 bp, Artificial
  Probe
ggttgtctttttaactgctggcaaa

SEQ ID NO: 1450 - DNA, 25 bp, Artificial
  Probe
tagtaagtggggtctttgtgggttg

SEQ ID NO: 1451 - DNA, 25 bp, Artificial
  Probe
aatgacatggttaatctggaactta

SEQ ID NO: 1452 - DNA, 25 bp, Artificial
  Probe
tcgctcagctccaacatggcaaaaa

SEQ ID NO: 1453 - DNA, 25 bp, Artificial
  Probe
caaggctgggccgggaagggcgtgg

SEQ ID NO: 1454 - DNA, 25 bp, Artificial
  Probe
aggatggttataactacactctctc

SEQ ID NO: 1455 - DNA, 25 bp, Artificial
  Probe
tacactctctccaagacagagttcc

SEQ ID NO: 1456 - DNA, 25 bp, Artificial
  Probe
aatacagaactagctgccttcacaa

SEQ ID NO: 1457 - DNA, 25 bp, Artificial
  Probe
cgggaagggcgtgggttgaggagag

SEQ ID NO: 1458 - DNA, 25 bp, Artificial
  Probe
tggtgtccttgaccgcatgatgaag

SEQ ID NO: 1459 - DNA, 25 bp, Artificial
  Probe
gaagaaactggacaccaacagtgat

SEQ ID NO: 1460 - DNA, 25 bp, Artificial
  Probe
gtgatggtcagctagatttctcaga

SEQ ID NO: 1461 - DNA, 25 bp, Artificial
  Probe
ggttgaggagaggctccagacccgc

SEQ ID NO: 1462 - DNA, 25 bp, Artificial
  Probe
ttcccagaagcggacctgaggaccc

SEQ ID NO: 1463 - DNA, 25 bp, Artificial
  Probe
caccttccaccaatactctgtgaag

SEQ ID NO: 1464 - DNA, 25 bp, Artificial
  Probe
gcagctgagcttcgaggagttcatc

SEQ ID NO: 1465 - DNA, 25 bp, Artificial
  Probe
gatggcgaggctaacctgggcctcc

SEQ ID NO: 1466 - DNA, 25 bp, Artificial
  Probe
gatgcacgagggtgacgagggccct

SEQ ID NO: 1467 - DNA, 25 bp, Artificial
  Probe
ccctggccaccaccataagccaggc

SEQ ID NO: 1468 - DNA, 25 bp, Artificial
  Probe
gaccacagtggccaagatcacagtg

SEQ ID NO: 1469 - DNA, 25 bp, Artificial
  Probe
cacggccatggccacagtcatggtg

SEQ ID NO: 1470 - DNA, 25 bp, Artificial
  Probe
acggccacaggccactaatcaggag

SEQ ID NO: 1471 - DNA, 25 bp, Artificial
  Probe
ggggcctgttatgtcaaactgtctt

SEQ ID NO: 1472 - DNA, 25 bp, Artificial
  Probe
gcaaaatgtcgcagctggaacgcaa

SEQ ID NO: 1473 - DNA, 25 bp, Artificial
  Probe
ggaacgcaacatagagaccatcatc

SEQ ID NO: 1474 - DNA, 25 bp, Artificial
  Probe
gggggagctcaaggtgctgatggag

SEQ ID NO: 1475 - DNA, 25 bp, Artificial
  Probe
gggtctgaatctagcaccatgacgg

SEQ ID NO: 1476 - DNA, 25 bp, Artificial
  Probe
gatgccgtggataaattgctcaagg

SEQ ID NO: 1477 - DNA, 25 bp, Artificial
  Probe
gacctggacgccaatggagatgccc

SEQ ID NO: 1478 - DNA, 25 bp, Artificial
  Probe
ggtggacttcagtgagttcatcgtg

SEQ ID NO: 1479 - DNA, 25 bp, Artificial
  Probe
gtgttcgtggctgcaatcacgtctg

SEQ ID NO: 1480 - DNA, 25 bp, Artificial
  Probe
cgtctgcctgtcacaagtactttga

SEQ ID NO: 1481 - DNA, 25 bp, Artificial
  Probe
gacggaactagagacagccatgggc

SEQ ID NO: 1482 - DNA, 25 bp, Artificial
  Probe
tttgttggcaattattcccctaggc

SEQ ID NO: 1483 - DNA, 25 bp, Artificial
  Probe
cctaggctgagcctgctcatgtacc

SEQ ID NO: 1484 - DNA, 25 bp, Artificial
  Probe
gatcatagacgtcttttcccgatat

SEQ ID NO: 1485 - DNA, 25 bp, Artificial
  Probe
aggcaacttctcataaaattcccat

SEQ ID NO: 1486 - DNA, 25 bp, Artificial
  Probe
taaaattcccatggttcttctcctt

SEQ ID NO: 1487 - DNA, 25 bp, Artificial
  Probe
tcttctcctttggctatttttcaaa

SEQ ID NO: 1488 - DNA, 25 bp, Artificial
  Probe
cctcattcacgctgacattctttgg

SEQ ID NO: 1489 - DNA, 25 bp, Artificial
  Probe
ggatacagccaaggcactggtacag

SEQ ID NO: 1490 - DNA, 25 bp, Artificial
  Probe
gaccagaggctggctatgtggctac

SEQ ID NO: 1491 - DNA, 25 bp, Artificial
  Probe
tggctacccccatagctatggttca

SEQ ID NO: 1492 - DNA, 25 bp, Artificial
  Probe
tggttcaggcagccatgactcttct

SEQ ID NO: 1493 - DNA, 25 bp, Artificial
  Probe
ctaagtgatgcttctcatctgccta

SEQ ID NO: 1494 - DNA, 25 bp, Artificial
  Probe
ctcatctgcctaaggcgggcggggt

SEQ ID NO: 1495 - DNA, 25 bp, Artificial
  Probe
gaaattcttctgtaagcctgtctga

SEQ ID NO: 1496 - DNA, 25 bp, Artificial
  Probe
tatttgccatcattagtacctctca

SEQ ID NO: 1497 - DNA, 25 bp, Artificial
  Probe
tagtacctctcaacttactttttag

SEQ ID NO: 1498 - DNA, 25 bp, Artificial
  Probe
aattctgagccattaatcctgctac

SEQ ID NO: 1499 - DNA, 25 bp, Artificial
  Probe
gccattaatcctgctacactttgaa

SEQ ID NO: 1500 - DNA, 25 bp, Artificial
  Probe
tcctgctacactttgaatgatacat

SEQ ID NO: 1501 - DNA, 25 bp, Artificial
  Probe
cagactaatctttgggggctttatt

SEQ ID NO: 1502 - DNA, 25 bp, Artificial
  Probe
aacatgttcaacactattattttgt

SEQ ID NO: 1503 - DNA, 25 bp, Artificial
  Probe
gagctatgagaattggtgctatcac

SEQ ID NO: 1504 - DNA, 25 bp, Artificial
  Probe
ggtgctatcaccattagctatttgc

SEQ ID NO: 1505 - DNA, 25 bp, Artificial
  Probe
ttagctatttgctgtaatgtcaaga

SEQ ID NO: 1506 - DNA, 25 bp, Artificial
  Probe
accagatgcaagaatgtaccttttc

SEQ ID NO: 1507 - DNA, 25 bp, Artificial
  Probe
cctcgccgtctgtgaattggaccat

SEQ ID NO: 1508 - DNA, 25 bp, Artificial
  Probe
ggaccatcctatttaactggcttca

SEQ ID NO: 1509 - DNA, 25 bp, Artificial
  Probe
ttttcagttggctgacttccacacc

SEQ ID NO: 1510 - DNA, 25 bp, Artificial
  Probe
ccacacctagcatctcatgagtgcc

SEQ ID NO: 1511 - DNA, 25 bp, Artificial
  Probe
tagcctgcgctgttttttagtttgg

SEQ ID NO: 1512 - DNA, 25 bp, Artificial
  Probe
tttatgagacccattcctatttctt

SEQ ID NO: 1513 - DNA, 25 bp, Artificial
  Probe
gtcaatgtttcttttatcacgatat

SEQ ID NO: 1514 - DNA, 25 bp, Artificial
  Probe
gaccttttatccacttacctagatt

SEQ ID NO: 1515 - DNA, 25 bp, Artificial
  Probe
caccacttcttttataactagtcct

SEQ ID NO: 1516 - DNA, 25 bp, Artificial
  Probe
tagtcctttactaatccaacccatg

SEQ ID NO: 1517 - DNA, 25 bp, Artificial
  Probe
ctcttcctggcttcttactgaaagg

SEQ ID NO: 1518 - DNA, 25 bp, Artificial
  Probe
caaggaagatggagctcccccatcc

SEQ ID NO: 1519 - DNA, 25 bp, Artificial
  Probe
cactgcactgccattgtcttttggt

SEQ ID NO: 1520 - DNA, 25 bp, Artificial
  Probe
ttgtcttttggttgccatggtcacc

SEQ ID NO: 1521 - DNA, 25 bp, Artificial
  Probe
gtgacggacttctgaggctgtttcc

SEQ ID NO: 1522 - DNA, 25 bp, Artificial
  Probe
tcctctgacttggggcagcttgggt

SEQ ID NO: 1523 - DNA, 25 bp, Artificial
  Probe
gggtgaggttcagcctgtgagggct

SEQ ID NO: 1524 - DNA, 25 bp, Artificial
  Probe
ggcccaaagggcagacctttctttg

SEQ ID NO: 1525 - DNA, 25 bp, Artificial
  Probe
gtgtggaccaaggagcttccatcta

SEQ ID NO: 1526 - DNA, 25 bp, Artificial
  Probe
tccatctagtgacaagtgaccccca

SEQ ID NO: 1527 - DNA, 25 bp, Artificial
  Probe
tccagggtggactctgtcttgttca

SEQ ID NO: 1528 - DNA, 25 bp, Artificial
  Probe
gttcactgcagtatcccaactgcag

SEQ ID NO: 1529 - DNA, 25 bp, Artificial
  Probe
gacatgcgtactgagcgctttgggc

SEQ ID NO: 1530 - DNA, 25 bp, Artificial
  Probe
gcgctttgggcagggaggtgcgggg

SEQ ID NO: 1531 - DNA, 25 bp, Artificial
  Probe
gggcctgtgggtggacagggtccta

SEQ ID NO: 1532 - DNA, 25 bp, Artificial
  Probe
gggtcctagaggaatggggcctgga

SEQ ID NO: 1533 - DNA, 25 bp, Artificial
  Probe
atggggcctggaactccagcaggat

SEQ ID NO: 1534 - DNA, 25 bp, Artificial
  Probe
gagaagagtacgaaggcccaaacaa

SEQ ID NO: 1535 - DNA, 25 bp, Artificial
  Probe
aaccccgattttagatgtgatattt

SEQ ID NO: 1536 - DNA, 25 bp, Artificial
  Probe
ttaggctttcattccagtttgtttt

SEQ ID NO: 1537 - DNA, 25 bp, Artificial
  Probe
atggatgttagcagtttattgacct

SEQ ID NO: 1538 - DNA, 25 bp, Artificial
  Probe
atgtccctcaagtttatggcagtgt

SEQ ID NO: 1539 - DNA, 25 bp, Artificial
  Probe
gtgtaccttgtgccactgaatttcc

SEQ ID NO: 1540 - DNA, 25 bp, Artificial
  Probe
gttggctgatattggagtgctcatt

SEQ ID NO: 1541 - DNA, 25 bp, Artificial
  Probe
gatattggagtgctcattcacatga

SEQ ID NO: 1542 - DNA, 25 bp, Artificial
  Probe
gagtgctcattcacatgaagtggat

SEQ ID NO: 1543 - DNA, 25 bp, Artificial
  Probe
atagatacttctcaagacatcacac

SEQ ID NO: 1544 - DNA, 25 bp, Artificial
  Probe
acttctcaagacatcacacagcgtg

SEQ ID NO: 1545 - DNA, 25 bp, Artificial
  Probe
gacatcacacagcgtgagtcaatca

SEQ ID NO: 1546 - DNA, 25 bp, Artificial
  Probe
gcgtgagtcaatcaaggagggaagc

SEQ ID NO: 1547 - DNA, 25 bp, Artificial
  Probe
ggagggaagccacaagcagactgac

SEQ ID NO: 1548 - DNA, 25 bp, Artificial
  Probe
gccacaagcagactgacaacgtttc

SEQ ID NO: 1549 - DNA, 25 bp, Artificial
  Probe
aatgaacgtttcattctcgttaata

SEQ ID NO: 1550 - DNA, 25 bp, Artificial
  Probe
gtttcattctcgttaataaaggcat

SEQ ID NO: 1551 - DNA, 25 bp, Artificial
  Probe
gagctgatgttaaaactcatttggt

SEQ ID NO: 1552 - DNA, 25 bp, Artificial
  Probe
ctcatttggtgaggtcaacgttgtc

SEQ ID NO: 1553 - DNA, 25 bp, Artificial
  Probe
gtcaacgttgtcacataccttcaca

SEQ ID NO: 1554 - DNA, 25 bp, Artificial
  Probe
gggatagtatattttgggttgcagt

SEQ ID NO: 1555 - DNA, 25 bp, Artificial
  Probe
tcaaacttgtgctcagactggtgaa

SEQ ID NO: 1556 - DNA, 25 bp, Artificial
  Probe
gttttcattctaattcaggtgtcta

SEQ ID NO: 1557 - DNA, 25 bp, Artificial
  Probe
attcaggtgtctacttattttatgt

SEQ ID NO: 1558 - DNA, 25 bp, Artificial
  Probe
cccccaccatgaagtttcttcctat

SEQ ID NO: 1559 - DNA, 25 bp, Artificial
  Probe
tttatgctgtaacttacccccaatc

SEQ ID NO: 1560 - DNA, 25 bp, Artificial
  Probe
ttacccccaatctttatctctggat

SEQ ID NO: 1561 - DNA, 25 bp, Artificial
  Probe
gttgactagcattttcaaaccttta

SEQ ID NO: 1562 - DNA, 25 bp, Artificial
  Probe
gagtttaagatacaggtcatccatc

SEQ ID NO: 1563 - DNA, 25 bp, Artificial
  Probe
gtcatccatcattcttaggctcact

SEQ ID NO: 1564 - DNA, 25 bp, Artificial
  Probe
tcattcttaggctcactttttacag

SEQ ID NO: 1565 - DNA, 25 bp, Artificial
  Probe
tgtttttccccagtactataacttg

SEQ ID NO: 1566 - DNA, 25 bp, Artificial
  Probe
ataacttgtggtttctgaactcatt

SEQ ID NO: 1567 - DNA, 25 bp, Artificial
  Probe
tcagattactcagttgccttacctc

SEQ ID NO: 1568 - DNA, 25 bp, Artificial
  Probe
tcagttgccttacctcatgggaaga

SEQ ID NO: 1569 - DNA, 25 bp, Artificial
  Probe
agcatgttagttacttggtttcaac

SEQ ID NO: 1570 - DNA, 25 bp, Artificial
  Probe
gaatggaaagagttgcccttgttgc

SEQ ID NO: 1571 - DNA, 25 bp, Artificial
  Probe
gcctgatttgattatgaagctgctt

SEQ ID NO: 1572 - DNA, 25 bp, Artificial
  Probe
aagctgcttaatcactcttcatgtg

SEQ ID NO: 1573 - DNA, 25 bp, Artificial
  Probe
agggacatgcttagcagtccccttc

SEQ ID NO: 1574 - DNA, 25 bp, Artificial
  Probe
gaaggatttggtccgtcataaccca

SEQ ID NO: 1575 - DNA, 25 bp, Artificial
  Probe
ataacccaaggtaccatcctaggct

SEQ ID NO: 1576 - DNA, 25 bp, Artificial
  Probe
atcctaggctgacacctaactcttc

SEQ ID NO: 1577 - DNA, 25 bp, Artificial
  Probe
ctttcatttcttctacaactcatac

SEQ ID NO: 1578 - DNA, 25 bp, Artificial
  Probe
ttcttctacaactcatacactcgta

SEQ ID NO: 1579 - DNA, 25 bp, Artificial
  Probe
tacactcgtatgatacttcgacact

SEQ ID NO: 1580 - DNA, 25 bp, Artificial
  Probe
cttcgacactgttcttagctcaatg

SEQ ID NO: 1581 - DNA, 25 bp, Artificial
  Probe
agctcaatgagcatgtttagacttt

SEQ ID NO: 1582 - DNA, 25 bp, Artificial
  Probe
tggtgttttttcctatgggtgttat

SEQ ID NO: 1583 - DNA, 25 bp, Artificial
  Probe
atgggtgttatcacctagctgaatg

SEQ ID NO: 1584 - DNA, 25 bp, Artificial
  Probe
tcacctgggcatggcatccaagaga

SEQ ID NO: 1585 - DNA, 25 bp, Artificial
  Probe
gaaagacgactcaacctggaactca

SEQ ID NO: 1586 - DNA, 25 bp, Artificial
  Probe
ctaaatgtgcccgaaagtccagatc

SEQ ID NO: 1587 - DNA, 25 bp, Artificial
  Probe
tggattttcactgttctaagtcccc

SEQ ID NO: 1588 - DNA, 25 bp, Artificial
  Probe
ccccgtgacctgtggtgaggcgaaa

SEQ ID NO: 1589 - DNA, 25 bp, Artificial
  Probe
acggatcctcagtgaagtgcattcg

SEQ ID NO: 1590 - DNA, 25 bp, Artificial
  Probe
ggaatgaccctaggagagctgctga

SEQ ID NO: 1591 - DNA, 25 bp, Artificial
  Probe
cttttgctctgtcctccaagaataa

SEQ ID NO: 1592 - DNA, 25 bp, Artificial
  Probe
gcaagtgaatttctactaccctctc

SEQ ID NO: 1593 - DNA, 25 bp, Artificial
  Probe
cctctcagtcaccatgttgcagact

SEQ ID NO: 1594 - DNA, 25 bp, Artificial
  Probe
tgcagactttccctgtctggaggct

SEQ ID NO: 1595 - DNA, 25 bp, Artificial
  Probe
ttcagtcaaattaacctgcgtgaat

SEQ ID NO: 1596 - DNA, 25 bp, Artificial
  Probe
aacctgcgtgaatatcccaatctgg

SEQ ID NO: 1597 - DNA, 25 bp, Artificial
  Probe
atcccaatctggtgacgatttacag

SEQ ID NO: 1598 - DNA, 25 bp, Artificial
  Probe
acagaagcttcaaacgtgttggtgc

SEQ ID NO: 1599 - DNA, 25 bp, Artificial
  Probe
gcttcaaacgtgttggtgcttccta

SEQ ID NO: 1600 - DNA, 25 bp, Artificial
  Probe
gttggtgcttcctatgaacggcaga

SEQ ID NO: 1601 - DNA, 25 bp, Artificial
  Probe
cctatgaacggcagagcagagacac

SEQ ID NO: 1602 - DNA, 25 bp, Artificial
  Probe
cagagacacaccaatcctacttgaa

SEQ ID NO: 1603 - DNA, 25 bp, Artificial
  Probe
agacacaccaatcctacttgaagcc

SEQ ID NO: 1604 - DNA, 25 bp, Artificial
  Probe
agccccaactggcctagcagaagga

SEQ ID NO: 1605 - DNA, 25 bp, Artificial
  Probe
caccctgtgcgccaagagtcagtga

SEQ ID NO: 1606 - DNA, 25 bp, Artificial
  Probe
ggattttcactgttctaagctcccc

SEQ ID NO: 1607 - DNA, 25 bp, Artificial
  Probe
ccccgtgacctgtggtgaggcgaaa

SEQ ID NO: 1608 - DNA, 25 bp, Artificial
  Probe
acggatcctcagtgaagtgcattcg

SEQ ID NO: 1609 - DNA, 25 bp, Artificial
  Probe
gaacgaccctaggagagctgctgaa

SEQ ID NO: 1610 - DNA, 25 bp, Artificial
  Probe
gaagagtggactttgctctgtcctc

SEQ ID NO: 1611 - DNA, 25 bp, Artificial
  Probe
gcaagtgaatttctactaccctctc

SEQ ID NO: 1612 - DNA, 25 bp, Artificial
  Probe
cctctcagtcaccatgttgcagact

SEQ ID NO: 1613 - DNA, 25 bp, Artificial
  Probe
tgcagactttccctgtctggaggct

SEQ ID NO: 1614 - DNA, 25 bp, Artificial
  Probe
gaggctcaccttagagcttctgagt

SEQ ID NO: 1615 - DNA, 25 bp, Artificial
  Probe
tgagtttccaagctctgagtcacct

SEQ ID NO: 1616 - DNA, 25 bp, Artificial
  Probe
tcacctccacatttgggcatggcat

SEQ ID NO: 1617 - DNA, 25 bp, Artificial
  Probe
tggtctaattgatggtcgcctcacc

SEQ ID NO: 1618 - DNA, 25 bp, Artificial
  Probe
catctgtacaatctcctgtttcttt

SEQ ID NO: 1619 - DNA, 25 bp, Artificial
  Probe
ctcctgtttctttgccatagtggct

SEQ ID NO: 1620 - DNA, 25 bp, Artificial
  Probe
tttgggattatatgcacccctttcc

SEQ ID NO: 1621 - DNA, 25 bp, Artificial
  Probe
tccagagtccaaacccgttttggct

SEQ ID NO: 1622 - DNA, 25 bp, Artificial
  Probe
ttggctttgtgtgtcatatcctatt

SEQ ID NO: 1623 - DNA, 25 bp, Artificial
  Probe
gagaagagcatctttctcgtggccc

SEQ ID NO: 1624 - DNA, 25 bp, Artificial
  Probe
tctcgtggcccacaggaaagatcct

SEQ ID NO: 1625 - DNA, 25 bp, Artificial
  Probe
gatgatatttggcagctgtcctcca

SEQ ID NO: 1626 - DNA, 25 bp, Artificial
  Probe
gaagccgagttcacaaagtccattg

SEQ ID NO: 1627 - DNA, 25 bp, Artificial
  Probe
tccaggctccatgacagtcttgcca

SEQ ID NO: 1628 - DNA, 25 bp, Artificial
  Probe
aagactaggtagatatggcatggcg

SEQ ID NO: 1629 - DNA, 25 bp, Artificial
  Probe
ccatacatccaacccatgttctgag

SEQ ID NO: 1630 - DNA, 25 bp, Artificial
  Probe
caacccatgttctgagcaactactt

SEQ ID NO: 1631 - DNA, 25 bp, Artificial
  Probe
tgagcaactacttacttttaggggg

SEQ ID NO: 1632 - DNA, 25 bp, Artificial
  Probe
aaatatcttttcatttcctcttcta

SEQ ID NO: 1633 - DNA, 25 bp, Artificial
  Probe
attttctaacaaggtttggccatag

SEQ ID NO: 1634 - DNA, 25 bp, Artificial
  Probe
taatcttctgtaggctatctttcaa

SEQ ID NO: 1635 - DNA, 25 bp, Artificial
  Probe
gagacttgggtttagttatagcttt

SEQ ID NO: 1636 - DNA, 25 bp, Artificial
  Probe
acttcgtatctaatggtttgtaaat

SEQ ID NO: 1637 - DNA, 25 bp, Artificial
  Probe
taaaccatttgcagagttgaactct

SEQ ID NO: 1638 - DNA, 25 bp, Artificial
  Probe
aatgttggtcataatactgctataa

SEQ ID NO: 1639 - DNA, 25 bp, Artificial
  Probe
cagctcatctcatgtcctgaagttg

SEQ ID NO: 1640 - DNA, 25 bp, Artificial
  Probe
gacagtgttggaatttggaggcagt

SEQ ID NO: 1641 - DNA, 25 bp, Artificial
  Probe
gaggcagtagttgagcatattctct

SEQ ID NO: 1642 - DNA, 25 bp, Artificial
  Probe
attctctagtatatagctacacctt

SEQ ID NO: 1643 - DNA, 25 bp, Artificial
  Probe
gtcttcaatcatattttagtgggct

SEQ ID NO: 1644 - DNA, 25 bp, Artificial
  Probe
agttgtacatttagccagtgttatt

SEQ ID NO: 1645 - DNA, 25 bp, Artificial
  Probe
atgttttggtactgtgttttcactc

SEQ ID NO: 1646 - DNA, 25 bp, Artificial
  Probe
gtgttttcactcaaaccactgactt

SEQ ID NO: 1647 - DNA, 25 bp, Artificial
  Probe
accactgacttaacagatactgctg

SEQ ID NO: 1648 - DNA, 25 bp, Artificial
  Probe
gatactgctgtgtataacatgtact

SEQ ID NO: 1649 - DNA, 25 bp, Artificial
  Probe
gattgttcctcttatatttgtgtgt

SEQ ID NO: 1650 - DNA, 25 bp, Artificial
  Probe
tgtcaaaaatcgtactaatgcttat

SEQ ID NO: 1651 - DNA, 25 bp, Artificial
  Probe
aaggttttcttgcataaatactgga

SEQ ID NO: 1652 - DNA, 25 bp, Artificial
  Probe
aatactggaaattgcacatggtaca

SEQ ID NO: 1653 - DNA, 25 bp, Artificial
  Probe
gcacatggtacaaattttttcttca

SEQ ID NO: 1654 - DNA, 25 bp, Artificial
  Probe
ctgaaagttactgaagtgccttctg

SEQ ID NO: 1655 - DNA, 25 bp, Artificial
  Probe
gaagtgccttctgaatcaaggattt

SEQ ID NO: 1656 - DNA, 25 bp, Artificial
  Probe
ggatttaattaaggccacaatacct

SEQ ID NO: 1657 - DNA, 25 bp, Artificial
  Probe
atacctttttaatactcagtgttct

SEQ ID NO: 1658 - DNA, 25 bp, Artificial
  Probe
aaaacttgatattcccgtatggtgc

SEQ ID NO: 1659 - DNA, 25 bp, Artificial
  Probe
gatattcccgtatggtgcatatttg

SEQ ID NO: 1660 - DNA, 25 bp, Artificial
  Probe
tggtgcatatttgatacaggtaccc

SEQ ID NO: 1661 - DNA, 25 bp, Artificial
  Probe
atggaaagtagcgcatccctgaggc

SEQ ID NO: 1662 - DNA, 25 bp, Artificial
  Probe
tctgactgcacgtggcttctgatga

SEQ ID NO: 1663 - DNA, 25 bp, Artificial
  Probe
caaaaccattgtcatgggcgcctcc

SEQ ID NO: 1664 - DNA, 25 bp, Artificial
  Probe
tccttccgcaacacgggcgagatca

SEQ ID NO: 1665 - DNA, 25 bp, Artificial
  Probe
atcaaagcactggccggctgtgact

SEQ ID NO: 1666 - DNA, 25 bp, Artificial
  Probe
cacccaagctcctgggagagctgct

SEQ ID NO: 1667 - DNA, 25 bp, Artificial
  Probe
aatccacctggatgagaagtctttc

SEQ ID NO: 1668 - DNA, 25 bp, Artificial
  Probe
gaagtctttccgttggttgcacaac

SEQ ID NO: 1669 - DNA, 25 bp, Artificial
  Probe
ggttgcacaacgaggaccagatggc

SEQ ID NO: 1670 - DNA, 25 bp, Artificial
  Probe
gctctctgacgggatccgcaagttt

SEQ ID NO: 1671 - DNA, 25 bp, Artificial
  Probe
ccgcaagtttgccgctgatgcagtg

SEQ ID NO: 1672 - DNA, 25 bp, Artificial
  Probe
agagcatttgtggcttgaacttgcc

SEQ ID NO: 1673 - DNA, 25 bp, Artificial
  Probe
gaacttgccagatgcaaataccaca

SEQ ID NO: 1674 - DNA, 25 bp, Artificial
  Probe
gaattcttatcttccagaggctaca

SEQ ID NO: 1675 - DNA, 25 bp, Artificial
  Probe
ggacaatacttttacctttgtctct

SEQ ID NO: 1676 - DNA, 25 bp, Artificial
  Probe
agttttatttgttcacttacgtgct

SEQ ID NO: 1677 - DNA, 25 bp, Artificial
  Probe
tcacttacgtgctttgattatcccc

SEQ ID NO: 1678 - DNA, 25 bp, Artificial
  Probe
gattatcccctctgaattatagacc

SEQ ID NO: 1679 - DNA, 25 bp, Artificial
  Probe
tcttctcaggtatggaaccacggtc

SEQ ID NO: 1680 - DNA, 25 bp, Artificial
  Probe
gaaccacggtcataactaacatgtt

SEQ ID NO: 1681 - DNA, 25 bp, Artificial
  Probe
gacaacaaattacctttctgggtgt

SEQ ID NO: 1682 - DNA, 25 bp, Artificial
  Probe
tcttgtaaactatactcctgtttga

SEQ ID NO: 1683 - DNA, 25 bp, Artificial
  Probe
tggcattctcctagctttaattgaa

SEQ ID NO: 1684 - DNA, 25 bp, Artificial
  Probe
gaaggagctggtatcttgttgacaa

SEQ ID NO: 1685 - DNA, 25 bp, Artificial
  Probe
tcccagttgccttcaactcagttac

SEQ ID NO: 1686 - DNA, 25 bp, Artificial
  Probe
caccttttggagactatcgacaata

SEQ ID NO: 1687 - DNA, 25 bp, Artificial
  Probe
gacaatatcagtaggacttctttcc

SEQ ID NO: 1688 - DNA, 25 bp, Artificial
  Probe
tctttcctaggatttctttaacaga

SEQ ID NO: 1689 - DNA, 25 bp, Artificial
  Probe
gagttgtggttcgagaaggatttca

SEQ ID NO: 1690 - DNA, 25 bp, Artificial
  Probe
agctatggccaataggctataaaga

SEQ ID NO: 1691 - DNA, 25 bp, Artificial
  Probe
aaagagacatttagcacttttttct

SEQ ID NO: 1692 - DNA, 25 bp, Artificial
  Probe
tgcctggttttgtgtgttctgttat

SEQ ID NO: 1693 - DNA, 25 bp, Artificial
  Probe
gctggtggaacttactctttctttt

SEQ ID NO: 1694 - DNA, 25 bp, Artificial
  Probe
taaaaggctacaggtctctttccac

SEQ ID NO: 1695 - DNA, 25 bp, Artificial
  Probe
tttccacatcccaaactttctatga

SEQ ID NO: 1696 - DNA, 25 bp, Artificial
  Probe
gacaccaaagatgcctctgttactg

SEQ ID NO: 1697 - DNA, 25 bp, Artificial
  Probe
gctgcgctaccaccttgaagagagc

SEQ ID NO: 1698 - DNA, 25 bp, Artificial
  Probe
caaaaacgttctcctttgtctagag

SEQ ID NO: 1699 - DNA, 25 bp, Artificial
  Probe
atcgacaacctcatgcagagcatca

SEQ ID NO: 1700 - DNA, 25 bp, Artificial
  Probe
gctttttacttggctttgcagaggc

SEQ ID NO: 1701 - DNA, 25 bp, Artificial
  Probe
ggagccagtgttacagcattctcag

SEQ ID NO: 1702 - DNA, 25 bp, Artificial
  Probe
ttgaggctacggcagcggatctgta

SEQ ID NO: 1703 - DNA, 25 bp, Artificial
  Probe
gcggatctgtaagagctccatcctc

SEQ ID NO: 1704 - DNA, 25 bp, Artificial
  Probe
tccagtggcctgacaacccgaaggc

SEQ ID NO: 1705 - DNA, 25 bp, Artificial
  Probe
tctttgggttattactgtctttact

SEQ ID NO: 1706 - DNA, 25 bp, Artificial
  Probe
gaaatgctgccctagaagtacaata

SEQ ID NO: 1707 - DNA, 25 bp, Artificial
  Probe
ttctggttgttgttggggcatgagc

SEQ ID NO: 1708 - DNA, 25 bp, Artificial
  Probe
gcttgcataaactcaaccagctgcc

SEQ ID NO: 1709 - DNA, 25 bp, Artificial
  Probe
agggagctctagtcctttttgtgta

SEQ ID NO: 1710 - DNA, 25 bp, Artificial
  Probe
gagagaacatccttgctttgagtca

SEQ ID NO: 1711 - DNA, 25 bp, Artificial
  Probe
gggcaagttcctgaccacagggagt

SEQ ID NO: 1712 - DNA, 25 bp, Artificial
  Probe
gggagtaaattggcctctttgatac

SEQ ID NO: 1713 - DNA, 25 bp, Artificial
  Probe
tctttgatacacttttgcttgcctc

SEQ ID NO: 1714 - DNA, 25 bp, Artificial
  Probe
attcatcgatgtttcgtgcttctcc

SEQ ID NO: 1715 - DNA, 25 bp, Artificial
  Probe
gtgcttctccttatgaaactccagc

SEQ ID NO: 1716 - DNA, 25 bp, Artificial
  Probe
gtagcagctcacataactgggacca

SEQ ID NO: 1717 - DNA, 25 bp, Artificial
  Probe
cattgtcttctccaaactccaagaa

SEQ ID NO: 1718 - DNA, 25 bp, Artificial
  Probe
tgggccgcaaaataaactcctggga

SEQ ID NO: 1719 - DNA, 25 bp, Artificial
  Probe
gggcattcattcctgagcaacttgc

SEQ ID NO: 1720 - DNA, 25 bp, Artificial
  Probe
ttttactacatctattcccaaacat

SEQ ID NO: 1721 - DNA, 25 bp, Artificial
  Probe
tcccaaacatactttcgatttcagg

SEQ ID NO: 1722 - DNA, 25 bp, Artificial
  Probe
aatacacaagttatcctgaccctat

SEQ ID NO: 1723 - DNA, 25 bp, Artificial
  Probe
ggactctattccatctatcaagggg

SEQ ID NO: 1724 - DNA, 25 bp, Artificial
  Probe
tgagcacttgatagacatggaccat

SEQ ID NO: 1725 - DNA, 25 bp, Artificial
  Probe
ggaccatgaagccagttttttcggg

SEQ ID NO: 1726 - DNA, 25 bp, Artificial
  Probe
gcctttttagttggctaactgacct

SEQ ID NO: 1727 - DNA, 25 bp, Artificial
  Probe
ctctacctcatatcagtttgctagc

SEQ ID NO: 1728 - DNA, 25 bp, Artificial
  Probe
aagactgtcagcttccaaacattaa

SEQ ID NO: 1729 - DNA, 25 bp, Artificial
  Probe
atgcaatggttaacatcttctgtct

SEQ ID NO: 1730 - DNA, 25 bp, Artificial
  Probe
tctgtctttataatctactccttgt

SEQ ID NO: 1731 - DNA, 25 bp, Artificial
  Probe
taatctactccttgtaaagactgta

SEQ ID NO: 1732 - DNA, 25 bp, Artificial
  Probe
agaaagcgcaacaatccatctctca

SEQ ID NO: 1733 - DNA, 25 bp, Artificial
  Probe
atccatctctcaagtagtgtatcac

SEQ ID NO: 1734 - DNA, 25 bp, Artificial
  Probe
agtgtatcacagtagtagcctccag

SEQ ID NO: 1735 - DNA, 25 bp, Artificial
  Probe
tcacagtagtagcctccaggtttcc

SEQ ID NO: 1736 - DNA, 25 bp, Artificial
  Probe
gaggcaccactaaaagatcgcagtt

SEQ ID NO: 1737 - DNA, 25 bp, Artificial
  Probe
atcgcagtttgcctggtgcagtggc

SEQ ID NO: 1738 - DNA, 25 bp, Artificial
  Probe
catggagctccgaattcttgcgtgt

SEQ ID NO: 1739 - DNA, 25 bp, Artificial
  Probe
gcattgttcagacctggtcggggcc

SEQ ID NO: 1740 - DNA, 25 bp, Artificial
  Probe
tcggggcccactggaagcatccaga

SEQ ID NO: 1741 - DNA, 25 bp, Artificial
  Probe
tagggaaaacccctggttctccatg

SEQ ID NO: 1742 - DNA, 25 bp, Artificial
  Probe
ccacaagaagccttatcctacgtcc

SEQ ID NO: 1743 - DNA, 25 bp, Artificial
  Probe
gagatgtagctattatgcgcccgtc

SEQ ID NO: 1744 - DNA, 25 bp, Artificial
  Probe
ctacagggggtgcccgacgatgacg

SEQ ID NO: 1745 - DNA, 25 bp, Artificial
  Probe
gtgccttcgcagtcaaattactctt

SEQ ID NO: 1746 - DNA, 25 bp, Artificial
  Probe
ttccaagcccttccgggctggaact

SEQ ID NO: 1747 - DNA, 25 bp, Artificial
  Probe
tcggaggagcctcgggtgtatcgta

SEQ ID NO: 1748 - DNA, 25 bp, Artificial
  Probe
ctgagctcttctttctgatcaagcc

SEQ ID NO: 1749 - DNA, 25 bp, Artificial
  Probe
agcaaattgagcttgggtgattttt

SEQ ID NO: 1750 - DNA, 25 bp, Artificial
  Probe
aaacgtggtaaatcacttcatatta

SEQ ID NO: 1751 - DNA, 25 bp, Artificial
  Probe
agtagcattagctttagttacaaat

SEQ ID NO: 1752 - DNA, 25 bp, Artificial
  Probe
ggatctttctgctgacaacttaggt

SEQ ID NO: 1753 - DNA, 25 bp, Artificial
  Probe
taaatctgatgtttcctgtacctgc

SEQ ID NO: 1754 - DNA, 25 bp, Artificial
  Probe
ctgtacctgccacactatgttagaa

SEQ ID NO: 1755 - DNA, 25 bp, Artificial
  Probe
atgtgtccttcaaacatatcctcct

SEQ ID NO: 1756 - DNA, 25 bp, Artificial
  Probe
ctcctgcaacttctcaaactgtact

SEQ ID NO: 1757 - DNA, 25 bp, Artificial
  Probe
tcttgaagtctaactctgtgctaac

SEQ ID NO: 1758 - DNA, 25 bp, Artificial
  Probe
ctgtgctaacagatctccattttaa

SEQ ID NO: 1759 - DNA, 25 bp, Artificial
  Probe
agatgtgaatgttaatcactgcttg

SEQ ID NO: 1760 - DNA, 25 bp, Artificial
  Probe
tttggaaaaaccttgcatacgcctt

SEQ ID NO: 1761 - DNA, 25 bp, Artificial
  Probe
gcatacgccttttctatcaagtgct

SEQ ID NO: 1762 - DNA, 25 bp, Artificial
  Probe
acagtatccttacctgccatttaat

SEQ ID NO: 1763 - DNA, 25 bp, Artificial
  Probe
acctgccatttaatattagcctcgt

SEQ ID NO: 1764 - DNA, 25 bp, Artificial
  Probe
cctcgtatttttctcacgtatattt

SEQ ID NO: 1765 - DNA, 25 bp, Artificial
  Probe
acgtatatttacctgtgacttgtat

SEQ ID NO: 1766 - DNA, 25 bp, Artificial
  Probe
aactgtagcgcttcattatactatt

SEQ ID NO: 1767 - DNA, 25 bp, Artificial
  Probe
agttttatctcttgcatatacttta

SEQ ID NO: 1768 - DNA, 25 bp, Artificial
  Probe
taaatgttaccagcactttttttgt

SEQ ID NO: 1769 - DNA, 25 bp, Artificial
  Probe
tgtaagtttcactttccgaggtatt

SEQ ID NO: 1770 - DNA, 25 bp, Artificial
  Probe
ggtattgtacaagttcacactgttt

SEQ ID NO: 1771 - DNA, 25 bp, Artificial
  Probe
attaagcattgccacatctaggaat

SEQ ID NO: 1772 - DNA, 25 bp, Artificial
  Probe
ggttacaggtgttacttctctgacc

SEQ ID NO: 1773 - DNA, 25 bp, Artificial
  Probe
tctctgaccttcaatcctactacag

SEQ ID NO: 1774 - DNA, 25 bp, Artificial
  Probe
gaagcagtcagattggttcatcttc

SEQ ID NO: 1775 - DNA, 25 bp, Artificial
  Probe
gtacagtattttcaaacttcccagt

SEQ ID NO: 1776 - DNA, 25 bp, Artificial
  Probe
gttcataccatggatttttctccga

SEQ ID NO: 1777 - DNA, 25 bp, Artificial
  Probe
gaagtggatactttgccttggggaa

SEQ ID NO: 1778 - DNA, 25 bp, Artificial
  Probe
aaggccctgatgtataggttgcacc

SEQ ID NO: 1779 - DNA, 25 bp, Artificial
  Probe
taggttgcaccattactcagacttc

SEQ ID NO: 1780 - DNA, 25 bp, Artificial
  Probe
gagaagcctgtcttgatatatcatc

SEQ ID NO: 1781 - DNA, 25 bp, Artificial
  Probe
gatccagctgtgcttaagagccagt

SEQ ID NO: 1782 - DNA, 25 bp, Artificial
  Probe
ggatcatctgggcctatactaacag

SEQ ID NO: 1783 - DNA, 25 bp, Artificial
  Probe
gtgggaccagcaattcagcaataac

SEQ ID NO: 1784 - DNA, 25 bp, Artificial
  Probe
gttataggggcgtctctttatcact

SEQ ID NO: 1785 - DNA, 25 bp, Artificial
  Probe
ctttatcactcagcttctgcatcat

SEQ ID NO: 1786 - DNA, 25 bp, Artificial
  Probe
cttctgcatcatacgcttggctgaa

SEQ ID NO: 1787 - DNA, 25 bp, Artificial
  Probe
ggctgaatgtgtttatcggcttccc

SEQ ID NO: 1788 - DNA, 25 bp, Artificial
  Probe
aatgaagatcaaactccagctccag

SEQ ID NO: 1789 - DNA, 25 bp, Artificial
  Probe
ccagcctcattttgcttgagacttt

SEQ ID NO: 1790 - DNA, 25 bp, Artificial
  Probe
gggagtgaggagtttcagggccatt

SEQ ID NO: 1791 - DNA, 25 bp, Artificial
  Probe
gccattgaaacatagctgtgccctt

SEQ ID NO: 1792 - DNA, 25 bp, Artificial
  Probe
ggtttatgactgaattccctttgac

SEQ ID NO: 1793 - DNA, 25 bp, Artificial
  Probe
gaacagacgagatctatgccctagg

SEQ ID NO: 1794 - DNA, 25 bp, Artificial
  Probe
atgccctaggtgcttttgatggact

SEQ ID NO: 1795 - DNA, 25 bp, Artificial
  Probe
gaacctgtggggtcagcttttacca

SEQ ID NO: 1796 - DNA, 25 bp, Artificial
  Probe
caagtttgaagacttctccctcagt

SEQ ID NO: 1797 - DNA, 25 bp, Artificial
  Probe
ttggaacgcgttatgttttcccaca

SEQ ID NO: 1798 - DNA, 25 bp, Artificial
  Probe
tctaagtgggagtcagcttgcccct

SEQ ID NO: 1799 - DNA, 25 bp, Artificial
  Probe
ttgaggagccgaagtggagcccctt

SEQ ID NO: 1800 - DNA, 25 bp, Artificial
  Probe
ttgcctgtcttagttatggccctgt

SEQ ID NO: 1801 - DNA, 25 bp, Artificial
  Probe
cctccacgcttagggcagggatctg

SEQ ID NO: 1802 - DNA, 25 bp, Artificial
  Probe
gaaattccagtgatctcctttagca

SEQ ID NO: 1803 - DNA, 25 bp, Artificial
  Probe
gcagagcccttttaggattagcctg

SEQ ID NO: 1804 - DNA, 25 bp, Artificial
  Probe
ctcctcaagctatccaattttctga

SEQ ID NO: 1805 - DNA, 25 bp, Artificial
  Probe
taaccatgagagatgccacatttct

SEQ ID NO: 1806 - DNA, 25 bp, Artificial
  Probe
ttctctctgggaaactaccactcaa

SEQ ID NO: 1807 - DNA, 25 bp, Artificial
  Probe
gcagatcacatgtaaatcattccta

SEQ ID NO: 1808 - DNA, 25 bp, Artificial
  Probe
tgtgccttgatgtacatatattact

SEQ ID NO: 1809 - DNA, 25 bp, Artificial
  Probe
tatattactaagttgcctctcccag

SEQ ID NO: 1810 - DNA, 25 bp, Artificial
  Probe
atgtgatagctgtgcatgcattata

SEQ ID NO: 1811 - DNA, 25 bp, Artificial
  Probe
agctgtgtggctgactttcaatttt

SEQ ID NO: 1812 - DNA, 25 bp, Artificial
  Probe
ttgacatacagcccataactttata

SEQ ID NO: 1813 - DNA, 25 bp, Artificial
  Probe
actttataatggctgctcatttatc

SEQ ID NO: 1814 - DNA, 25 bp, Artificial
  Probe
catcctctgttgttactagatttag

SEQ ID NO: 1815 - DNA, 25 bp, Artificial
  Probe
agttttgcacttttatagcctattt

SEQ ID NO: 1816 - DNA, 25 bp, Artificial
  Probe
acacatttgcaagatgattgactca

SEQ ID NO: 1817 - DNA, 25 bp, Artificial
  Probe
attgactcaatctttgcctaatcca

SEQ ID NO: 1818 - DNA, 25 bp, Artificial
  Probe
ttgcctaatccaatgagtgttacag

SEQ ID NO: 1819 - DNA, 25 bp, Artificial
  Probe
gagagcttgctgtgactagaaccat

SEQ ID NO: 1820 - DNA, 25 bp, Artificial
  Probe
ttcagatttctctttttaaccacat

SEQ ID NO: 1821 - DNA, 25 bp, Artificial
  Probe
ttcctacagccctttgtacttcaaa

SEQ ID NO: 1822 - DNA, 25 bp, Artificial
  Probe
atatgtttttgtgtccatcagtatt

SEQ ID NO: 1823 - DNA, 25 bp, Artificial
  Probe
ttaactattggtatactactggttt

SEQ ID NO: 1824 - DNA, 25 bp, Artificial
  Probe
agaggtacaattcgttggatttttg

SEQ ID NO: 1825 - DNA, 25 bp, Artificial
  Probe
gacattacgtgttttatttatgata

SEQ ID NO: 1826 - DNA, 25 bp, Artificial
  Probe
accatggggtgagtgtcctccaaga

SEQ ID NO: 1827 - DNA, 25 bp, Artificial
  Probe
gcttggaggcgagcattttcactgc

SEQ ID NO: 1828 - DNA, 25 bp, Artificial
  Probe
ttcactgctaggacaagctcagctg

SEQ ID NO: 1829 - DNA, 25 bp, Artificial
  Probe
gattttaaccattcaacatgctgtt

SEQ ID NO: 1830 - DNA, 25 bp, Artificial
  Probe
gaattgctactgaaagctatcccag

SEQ ID NO: 1831 - DNA, 25 bp, Artificial
  Probe
gctatcccaggtgatacagagctct

SEQ ID NO: 1832 - DNA, 25 bp, Artificial
  Probe
gagctctttgtaaaccgcagtcaca

SEQ ID NO: 1833 - DNA, 25 bp, Artificial
  Probe
aatgccagtctggtcagggaagtag

SEQ ID NO: 1834 - DNA, 25 bp, Artificial
  Probe
ccaggaaggtgggacagccggcagg

SEQ ID NO: 1835 - DNA, 25 bp, Artificial
  Probe
gtagggacattgtgtacctcagttg

SEQ ID NO: 1836 - DNA, 25 bp, Artificial
  Probe
gtgtacctcagttgtgtcacatgtg

SEQ ID NO: 1837 - DNA, 25 bp, Artificial
  Probe
aaagctgtgaatctgttccctgctg

SEQ ID NO: 1838 - DNA, 25 bp, Artificial
  Probe
ttccctgctggaacaaattcaagat

SEQ ID NO: 1839 - DNA, 25 bp, Artificial
  Probe
gagtggtacctcaagcagacaacgc

SEQ ID NO: 1840 - DNA, 25 bp, Artificial
  Probe
acaacgcaacgccttcagaacgatt

SEQ ID NO: 1841 - DNA, 25 bp, Artificial
  Probe
aggtccatatacagacttcacccct

SEQ ID NO: 1842 - DNA, 25 bp, Artificial
  Probe
gacttcaccccttggacaacagaag

SEQ ID NO: 1843 - DNA, 25 bp, Artificial
  Probe
acaagctttgaaaacatacccagta

SEQ ID NO: 1844 - DNA, 25 bp, Artificial
  Probe
aacatacccagtaaatacacctgaa

SEQ ID NO: 1845 - DNA, 25 bp, Artificial
  Probe
atagcagaagcggtgcctggcagga

SEQ ID NO: 1846 - DNA, 25 bp, Artificial
  Probe
gaaggactgcatgaaacgatacaag

SEQ ID NO: 1847 - DNA, 25 bp, Artificial
  Probe
gctgctcaagaacaagtgctgaatg

SEQ ID NO: 1848 - DNA, 25 bp, Artificial
  Probe
aatgcccatggtactccatgcattc

SEQ ID NO: 1849 - DNA, 25 bp, Artificial
  Probe
tgcatgtctggactcacggagctca

SEQ ID NO: 1850 - DNA, 25 bp, Artificial
  Probe
gtgttgcaagttggtccacagcatc

SEQ ID NO: 1851 - DNA, 25 bp, Artificial
  Probe
ccacagcatctccggggctttgtgg

SEQ ID NO: 1852 - DNA, 25 bp, Artificial
  Probe
gctttgtgggatcagggcattgcct

SEQ ID NO: 1853 - DNA, 25 bp, Artificial
  Probe
gaagtccatattgttccttatcacc

SEQ ID NO: 1854 - DNA, 25 bp, Artificial
  Probe
ggcccctggacgaaggtctgaatcc

SEQ ID NO: 1855 - DNA, 25 bp, Artificial
  Probe
aaggtctgaatcccgactctgatac

SEQ ID NO: 1856 - DNA, 25 bp, Artificial
  Probe
gctagagtttccttatccagacagt

SEQ ID NO: 1857 - DNA, 25 bp, Artificial
  Probe
gaaattggcgatgtcacccgtgtac

SEQ ID NO: 1858 - DNA, 25 bp, Artificial
  Probe
gcagaccctcaataaacgtcagctt

SEQ ID NO: 1859 - DNA, 5406 bp, Homo sapiens
gtattttggtctaaagtgtgatgagtatttcaatatgtgaaaactactagaatataatagggtctaacttgagaaattctttgggaaaatggtttctgatagttttatttcacgagtctcccctatttagaatattgtgatgcaagagaagaaagcgtttggattatagaatctcttgacagtgtggtggttccacctgcccagtgtggctttgaaattatgactagagaaaatcttttaaagtggacatttactgatttatagaggggcccacagatgagcttctgagatctgtaactcttgaagccttcaccacacatccttctaaaaccgtatatttaactgctgcttcccaaaggaatgtgatctgaaatgggtgaagaaatcattttgtagaagttgatctgtatataaaattatagaagaaagaagtaaatttagtagtcattcttaaccttaaaatcttgctgacttttgactgtttgtcatggtatactagacattgctcaagtgaatcccccctctagtgttaagggcatttactcatgttgaacctagttttatttacagtatatttgtatgcatagaagatggaggtccaccaaagtgttaattatgcttagttgtaggtcaggtatagctaactttccttttttaatatatatatttacatttgtgtttcctttataatttatggcatagattgccacgattttcttaagtatacttttataatcagaaaaatgatattaaggactcattttaagtacactaaatcaaatattagaaggcttctttattttaagctaattgtgaggattatttgtcatttaaaacttttgcttctacttattaccctgaagtatctttgtggtgcttatgtttttcacagactgtataaattgatatactctcccgccccatggtaatgttgctacacataagctctaataattatcatttttaatgttttaagattaattcaactaagttttaaaaataatccattggttacatacataagaaagtactgtatacagattcccctgacttataatggttcgacttaagattttttcaactttaccatgatgtgaaagccatatgaattcattgtgctcctcgatttatgatgggactacatccaggtgaagtcattgtaaattggaattgttgtaagttcaaaagtcactttttgatttaaaatacgtgtaacttacactgggtttatcaggatgtaacatcacaagtcgtggagcatctgtatttcggtcatttaatggatgatatctgactgaagggagaaaatgaatataaaaggcatgaaaacaggaatagaaaaggcatgtttaaagttctcagcgcagggctgataactctagctgctctctggaggtggtgttaggattttgttgttttttagttaaggatttcccactggaaaaatgtaggtctgcttattacagtatgttttcaaatttctaataccctgcctttcccactgggaccttatttgaaatagttgagttaactttagtcttgtgtcaaatagtactctttgaagtcatggctgatgtttattgagagttgactgtactagtttcagcttttttttttttttttttttgagacagagtctcactctgttgccagattggagtgcagtagtgtgatcttggctcactgcaacctcctcctccttgcaacctctgcctccgcctccctggttcaaatgattctcctgccttagcctcccgagtagctgggtctacagggacatgccaccacgcccagctaatttttgtatttttagtagagacgggttttcaccatgttggccgggatggtctcgatctcttgacctcgtgattcacccgcctcggcctcccaaagtgctgggattacaggtatgagccaccatgcccgtcctttttttgagacagggtcttgctctgtcgcccaagctggagtgtggtggcgtgatcttggctcactgcaacctctgcctcccaggtttgagccgttcttgtgcctcagcctcttgagtagctgggattataggcgcatgctatgacacccagctaatttttgtattttttttgtagagacggagttttgctatgttggccagggtggtgtccttgacctcaagtgatctgcctggcttggcctcccaaagtggtgggattacgggtatgagccaccacacttggccttagactttctcttactttatatatatttaatctcagtccttaaaataactgggtagataggaagaaactgagacagaaattaggtaaataaggcccaaggcagttaaataggataaaagcctggactgactgtgcttttaactcactaccctgtactgtgaaaatttacctatattaattatagaatcttaaaattctggactgagtgtaagcagtatgatgtaggtaatgactttagaattcaactgcagtaagtaggttaatattgtaagaactaatttgcttttctgaagtaatttaaaatgtgtgaatatcctatatgaggggccttaaatataactcactcagttctttctcaaagaaaatgagaaaaggaatggtaaatgttaacttgcaggcctcttttttttgttttttgttttttgactcttaaagcactttacttatttttaaaatttaattaattttttgtagagatggggtctcactttgttgcccaggctggtcttgaactcctaggttcaagtgatcctcttgcctcagcctcccaaagtgctgagattacaggtgtgagtcaccgtgcctggccttttgaagcactgtaaaacctgaatatatgggtagtgaggatataatcggaaccagaataaggattgtttttaaatactgagttcttcagtgtactgtgaagtgctgggaggtactactaaaatgtatctcttcttttctcttcattattaatgctactgccaaggttagctcctcccctgactgttagaatatttcgttacttctgtgggaattacttctttcatgctgcttatgagaagttgtgtgtgtgtgtttgtgtgtgtgtgtgtgtaccatttcttttcagataagtggatattcaatatgatagaattgaaatgctaaagaactataaggaaggcctttttcagtctcactcaaaccttttttcagtgtggttaccggttcttgcacccaccctggttgcttaccatattgcagctttgttacttgaatagtatttcagtttttaacacatttgtttttgtgttggttctgtttcctagtatggctgttttttttgttttgttttgagatggtgagatggggtattactctgtcatccaggctggagtgcagtggcatagtcatggctcactgcagccttgaactcccaggcccaagtgatcctcccacctcaacctcatgagtagctgggaccacaggtgtgcaccccatgcccaactaattatttttgtagagataggatctcactgtgttgcccgggctggtctcaaactcctggcctctagtgatcctcccgctttggactcccaaagtgctgggattataggtctgagccaccatgcccagtcagcgtttatcagattacttaccattactactttgtcctggggaaatcctcttaatctttaaaggcgcaatccaaaatcataatgttcccgtgttacttactgttacttttttttttttctcctatagtggtttgatgataagaacccaatttgggccgggcgcggttgctcacgcctgtaatcccaacactttgggaggctaagccagatggatcacctgaggtcgggagtttgagaccatcctgaccaacataaagaagtcctatctctactaaaaacacaaaattagctgggcgtggtggtgcatgcctgtattccagctacttgggaggctgaggcagaagaatcgcttgaacccaggaggcggaggttgcagtgagccgagatcgcgccattgcactccagcctgggcaacaagagcgaaattccgtctcaaaaaaaaaaaaaaaattggagttttacagataaccacatcttattctgggaaaggatttgaagcaagttgggttttatatttggctgtacttgtcctcttcagcagtataataagccccttaaggctgaagtaaccttattcctattgtttagtagctaatagcatgcttttgatatgcttatgatcatactaataatttaatatttgaattgtatggaagtacaattcagtatcattttacatatggtatattgtgatgctgtatcatattttatgttacggtttataagaaaagctcctaggtataaaatgctacatagcaggaacttggtttttcaatgttattatttcctactgtttttgacgtaacggcaataaaatttgtttgaaccaaaatggactaacaattatttgtacaactcagtattgtctaaatatcatattgttaaatctaggtttcttgaattctccatcaagcctggtcatgtcatgtagcatttggtgtctcaccatgcccaacagatattttgtgggaggatggagttgatcttcctcatgttaaaagattgaagggagtgttctgacttaattgataacagtctttcataacttcacaaatttttgagaatgacccaaggctaactgtgggaaaaattcacataaaaacatagcctatctatgaggagcaaaactatatttcagttgtgggctttacatttcatttaaccctcttaactgtcctgtgaaatgggttacagccttattttatagatgaggaagctgaagtttaagggatttgcatacggtcacgtaactagtgagttgtgcagctagggttagaataaacagatttattttttttttttcttagaaacagcaattaacaatgtgactcctaatcaaaagaaaagagatgtccttggggcttaaagtactatggtgggagtcttggactgagtaggtttgaaaatacaattttatgatcgtggagtactaggatttagtcattttgatgcagagcatttcctgatcaactgctgttgtggagtgtactgtccaatagaattctctacaattaaggaaatgttctgtatctcaagagattgttcttaatggtggccagtagtcatgtgaccgttgagcatttgaaatgtggctagtgctactgaagaatggaattgtaaattgcttttaatctaaattttgcctgtgatattattggctgtgggtttgccaaaatttgtttttttaaagaggaaaagataacggactgttggctgctttattggacagcacagctagcatatagatgcagataggtagtataacttgtttgtagtttaatataaatgttgtattttgtaattag

SEQ ID NO: 1860 - DNA, 2651 bp, Homo sapiens
tcagcctcccaaagtgctgagattacaggtgtgagtcaccgtgcctggccttttgaagcactgtaaaacctgaatatatgggtagtgaggatataatcggaaccagaataaggattgtttttaaatactgagttcttcagtgtactgtgaagtgctgggaggtactactaaaatgtatctcttcttttctcttcattattaatgctactgccaaggttagctcctcccctgactgttagaatatttcgttacttctgtgggaattacttctttcatgctgcttatgagaagttgtgtgtgtgtgtttgtgtgtgtgtgtgtgtaccatttcttttcagataagtggatattcaatatgatagaattgaaatgctaaagaactataaggaaggcctttttcagtctcactcaaaccttttttcagtgtggttaccggttcttgcacccaccctggttgcttaccatattgcagctttgttacttgaatagtatttcagtttttaacacatttgtttttgtgttggttctgtttcctagtatggctgttttttttgttttgttttgagatggtgagatggggtattactctgtcatccaggctggagtgcagtggcatagtcatggctcactgcagccttgaactcccaggcccaagtgatcctcccacctcaacctcatgagtagctgggaccacaggtgtgcaccccatgcccaactaattatttttgtagagataggatctcactgtgttgcccgggctggtctcaaactcctggcctctagtgatcctcccgctttggactcccaaagtgctgggattataggtctgagccaccatgcccagtcagcgtttatcagattacttaccattactactttgtcctggggaaatcctcttaatctttaaaggcgcaatccaaaatcataatgttcccgtgttacttactgttacttttttttttttctcctatagtggtttgatgataagaacccaatttgggccgggcgcggttgctcacgcctgtaatcccaacactttgggaggctaagccagatggatcacctgaggtcgggagtttgagaccatcctgaccaacataaagaagtcctatctctactaaaaacacaaaattagctgggcgtggtggtgcatgcctgtattccagctacttgggaggctgaggcagaagaatcgcttgaacccaggaggcggaggttgcagtgagccgagatcgcgccattgcactccagcctgggcaacaagagcgaaattccgtctcaaaaaaaaaaaaaaaattggagttttacagataaccacatcttattctgggaaaggatttgaagcaagttgggttttatatttggctgtacttgtcctcttcagcagtataataagccccttaaggctgaagtaaccttattcctattgtttagtagctaatagcatgcttttgatatgcttatgatcatactaataatttaatatttgaattgtatggaagtacaattcagtatcattttacatatggtatattgtgatgctgtatcatattttatgttacggtttataagaaaagctcctaggtataaaatgctacatagcaggaacttggtttttcaatgttattatttcctactgtttttgacgtaacggcaataaaatttgtttgaaccaaaatggactaacaattatttgtacaactcagtattgtctaaatatcatattgttaaatctaggtttcttgaattctccatcaagcctggtcatgtcatgtagcatttggtgtctcaccatgcccaacagatattttgtgggaggatggagttgatcttcctcatgttaaaagattgaagggagtgttctgacttaattgataacagtctttcataacttcacaaatttttgagaatgacccaaggctaactgtgggaaaaattcacataaaaacatagcctatctatgaggagcaaaactatatttcagttgtgggctttacatttcatttaaccctcttaactgtcctgtgaaatgggttacagccttattttatagatgaggaagctgaagtttaagggatttgcatacggtcacgtaactagtgagttgtgcagctagggttagaataaacagatttattttttttttttcttagaaacagcaattaacaatgtgactcctaatcaaaagaaaagagatgtccttggggcttaaagtactatggtgggagtcttggactgagtaggtttgaaaatacaattttatgatcgtggagtactaggatttagtcattttgatgcagagcatttcctgatcaactgctgttgtggagtgtactgtccaatagaattctctacaattaaggaaatgttctgtatctcaagagattgttcttaatggtggccagtagtcatgtgaccgttgagcatttgaaatgtggctagtgctactgaagaatggaattgtaaattgcttttaatctaaattttgcctgtgatattattggctgtgggtttgccaaaatttgtttttttaaagaggaaaagataacggactgttggctgctttattggacagcacagctagcatatagatgcagataggtagtataacttgtttgtagtttaatataaatgttgtattttgtaattag

SEQ ID NO: 1861 - DNA, 3935 bp, Homo sapiens
ctctgcttctgagaccctcctgttactgttatcatcgttccctagcctggctctgcctttctcagcagcccacattccatggatgggagcaggggggcagggacccaaaggagggaaatggctgtgggtggtgtgaaggccccccagccctcaggaaggtggggcaagagaccactgagcacaagggatcttgcccacctcctctttgactctgtggattatccatccatctgctcactgtgaagatggagaggcagtgccctaaggctgttcaatagcttttccatattttttcaacattgaaaaaataatttttaaaaactgtgatttttttaaaaaatcatttggctggagggaagggaaaagggaaacaccaaaagctgtaccatgatgaactggagatatttaactggggcactttccagaccaagacaaacaaattcctttctggactctaaagcagccgaatcttgagactgtcaatgacagaaagctgaagagaggcctctatttcttcctttttcctttcttctgtctaaaaactctctcttgttccccttttccagcttcccttggactactgccccaatggccccttggactcgcgtttcatgtatgcgagcacacacacacacaaacttgcaaaataccgtttttcttaaggattgtgggaccgaataatatcacgtgccttcatcttttccttttatagttagatgaacctcttcctctttacaatttttttaaaaagtgataggggaggttgatgtgttagtggaagatttgggcatcgtttgagaagtaacttttgtttaacacattccccctaaacattgaacacaaacatttcaaccccttcatgacactctttggacatttaaagcattgagtaaccatgtacatgacagcctaaatccgtttgatttcagagcatttcctgaacattgtatttcatagacttctctgattttttcaaaaatgaggtgagcaatggcaagcagccttgttctcccaatttggtgcttttgcttttggtgtggggtgggcatggggggttgggggtggtgtgggtgtgtttagaaaaaagatgcattcctgaagatctctggtgctgaagggcctcgagttcctttcagagactgtatttgacacactttaggtacacacaaacgaatggtatcacatgcaatattttaatggagcaatgggagaggctctttgaaatggggtttgcatctttttgtaacattttgatttctctggtgccttattcctacttgatgctggcactcacatacccacaagaagctgacacagaagtcagccttaggcgtggggacatatgggtgatgtttgagcatgcaggggccatggggagtttggtgtcagttggtggagaagggactagatggcatctcttagccgaggccaacaggaactgcacaagtccattatagtcaaagttagcaattttgatacgtaaacacaatacttcattcttcctcatctgagctttccttccttcttccttttctatctctaccttctcataaaggtgctgctgctgctgctaaggtgcccggagtccagaatgtccattaatcactcaggcacgagcctggcactgccacgtcagcccccagcatgaccaaacccaggtttctcttgcttggggctgagaactgtcagatttttctcatcaaaaatgttttccaaggaatcagtggattacagtttttctgcattgaaaatgcactttaaaaaataaattaaagctccagactgtttaaaatatacagagggagcaggggaaagttaagcatgtgctagtgtctgaacccagttcagtttatctccagttgaaacgatatacactatattatgtataaatgtatacacacttcctatatgtatccacatatatatagtgtatatattatacatgtataggtgtgtatatgtgcatatatacacacatgcacataacaaaatcagatgctcattacaaatccagatgctcattacaaaaccagatgctacacaaacagcagcagaggaaacaaggttggactcttgcaacagatcacaaaaaataaaaacagctacttgcagtgactttggtcatttctgtatgttcataaagaatggattgtaacaaggaaaaaaaggaacagtgttagtgaaaaaggaaaaatgggcgaaaccatcttgatccgatgcgaatgcagtaatgttctatataccatttcatcagttatttcttttagtcatgttgatttgatttcagtttctggctatgaaaaacatttttaaactcgtcacccacaacaaactgaacaaaactactacagtgaaagcccttttcagtgaaagatgtcagaaacctcaaaacctttggcctgactcagaactaccatgtgaaaatcagtactctcttaatgtttgaaataaaaactgaaaaaaaaaacaaaaaaacaaaaaaccttttttgaagcaccttaacgtggccatccatttgagaagtgggtgccacttttttctttgagcaccttattgatgtgtttgctatctgctgtctttctgttacctgttggctgaatggctagctgttaacatatacatgtgcacagaagagatatctgggcatgtatgttctcaatgaagtttactgtggtgactgctgaaaggtgaacccatttcctgattttcccgccgcagtgttgtgataagattcgaagaaacctttttccctgcacagaaatgtttcttatcacattgtatcttagtatggaaaggaatatggtcccttttttgcaattgctactgtgtacacacacacacacacacacacacacacacacacacactgtatgtttagacctaaaatacacacacccacgcacacactgtatgtttatgtgacctaaaacatacacacatgcacacacacatacatatccattcattcattcattcaagtggtgtttccagtgtctgtgtgtcactgtttatgcagtttccatttcccagtgaattatgagtggagggcaacttttctaaccagattgtcttttcagaacaaagacctgggaattgaggaagagtttggaaagagggagaggcaaggaaagagagctttaaattgaaaggttaatttcctaagaggaacctgggctgaatgactgcagtgttataccctccaatctttgcaggtgggcatggaacactgcttgtatcactctgtgcacggtataaatccatatatccacaaaaacacacatccatccatcaacatatacatggtttgggatgagcaggtcaatagttttgagagggagtttgttcctttttttttctcattatactcttaaattgttgtcagttatcaaacaaacaaacagaaaaattgtttggaaaaaccttgcatacgccttttctatcaagtgctttaaaatatagactaaatacacacatcctgccagttttttcttacagtgacagtatccttacctgccatttaatattagcctcgtatttttctcacgtatatttacctgtgacttgtatttgttatttaaacaggaaaaaaaacattcaaaaaaagaaaaattaactgtagcgcttcattatactattatattattattattattgtgacattttggaatactgtgaagttttatctcttgcatatactttatacggaagtattacgccttaaaaatacgaaaataaattttacaaggtttctgttttgtgtggaagagtaattgatgttgctaagaatgatgtttgtttttttggggtttttgttgttttttttttaaatgttaccagcactttttttgtaagtttcactttccgaggtattgtacaagttcacactgtttgtgaagtttgaatatgaaggaataattaaaaaaaaaaaaactctt

SEQ ID NO: 1862 - DNA, 8439 bp, Mus musculus
aaatcttagaagcaatcggggttgacagcgctttcgtaattactaatgagaggatcttgtgctaccggaagagcaatagactgtgtggcgactcaaacaagtgtggggatgctgaggggctcctccagagtcccggatgacagctcttggaaacccttgtttgctaagaatcacagcccttgtaaacacctaatgttgagtttctttgaacactgtcccacctgaggggattcgtttggaaagcttccatttcaggcctctttaacagagtatcaatctgatgctttctccttcctccttatgataggtctcattctactttcccatgtcagagtttctttttatatatacaaaagtgccagccttgctagtttaaccctacagagaccattcagaactaacttaagcagcaacttaggagaactcaaagcattatctgtatttcaagcaggctcctgaatcagatctcatagcagatgcctgggaatgcgtggtgggaaagcactaacaggacatggagacacccaaccaaagctatgagaggaaacagttgacctttaaaacagtctcaccttaactttccttgaggcattggggacaagtttttcttgaaacttgcatatccactccagttccttcaccaaagattttcttctccagagcccagcctcctttctcccaggcagaaccataacaggcctgagggtgtccttgcagtggtccacagagttcaccttctgttcacaggggtatttacagaccttatagtagaagggtttccaaacagtctgtatggaaaacatacacagtactacttggcactgcgagctttgtgagactcatctgttgcctggaggcttgtagtcagaaatatccatggaagggagagtgcgaagtcatttagagccaaacaggaccgctggtgagaggatcattgggcagtatgagtcaagagcagatcaaggctccgtgtgcccagggccaatggcagtggcctatgaggatgttagacaacacatcaatggagtcacattctgagaagctaaagtgtgggctttgctgtaatggctgacattgttgaaatgttcgtgccacagcaagggaactacttggaagtagacctgttgtgatagtgccttcttgttgtagcaagtcatttattcagttaggcttttctggaccattgcccccatcttctgaagaggtctgagatgaagggataggacactgcccctgaaatgctgtgatttgaagatatttgcactagattctatcctctcctttaaactggagcaactgaatgagaggggaaaaattaacaaggacagctcaaaatgaaaagaaacccaaagtaatgtgttctgataacattatctcccctcactgctacatctttcctccccccttcctcccttccctcctagatctacttttttttccttcctcttaaaggaaacttccattttcttattaccaaatccaacaattacttctctttgtttctccccagtactgaatcataagcttattaatcactcatgagctaggaatatctagtaaagagcctctgccttgacagtgttgctggccttctctgtccatcacgggtgaacaacgaggggtaatagggagactagactggcccagctcttatggaagccagagtctggatttcacacctataaggagatgacacctatttacccagaacacatagtctgcagctcatcttaaaagacgcttaggaacaaaaggaagttcctgtgttacagcaaacagatgcggtagtacccaaagcttacctgtctcttctctctcctctcttccgtcttactgccatgtcctctcaacgagacttaaacttcatctcatgaatggcaccagaagaactatttgactccttggcttctctctttttcagtaggctggtagctcattcaaaattaaaaccaagcaaatactattagtggctagccccctgagggctgaacaatttcccaagtgtcttgatgatcccaatatcttgataatcaactctgataacttggaagttttggctggctcagacatctgtcaactttattttcattttgtctccatttccatttgaatcttaagtgagagtggaaaggtagaatcatgggaaagattgtgaggctgcaattctagggtagagtttgtcagaagtttgtattatcccaaatagaaatttctatacttactttcaatttaatgttaccctgaatataatttctattacatttattgttatttttataaaaatagagttcaattactatgtctagttgagtgctctcttttctattttcccacatggatgcagtaccaacctgttacctaaatatctttttattatattgttaatatgtaattctactgtagaccaaaaatataaaaacaaatttgctcattttaaacatatacagactctaatgagtaaagatgaggagaaaagaccagagagcagtggttgactatgttgttagaaatcaaagagtagccttacctatttttaaccagtgcttgccgtcacaccatagttaggactatgttagcatggcttcttcatgcttacgttctgcaagccttgtctgtctgtttcctttgatgtgttcgaggttgcacaatgatgctattgtttttttcttttggtaatgcctgattttattataatgtactttatcagtcatttcctttagaagaatgagggggaaagttttatttcttcttttaatttaaattttgtttaatgcactggaaataaaattggacacatttcactgtttaaaaatcagaaacgaaacaaaacaaaaccccgaagaaaaaaccagcaaacaagtaagtaataggatacacacacatacaaaaaagctatgaaaaatattctgttcatacaaaatataggctatatctcacatgagagataaatactgtcaagtaataaaaagacattgtcaactacagtgctgaaaactataagaggaacctaggtgtacagtgtgtggggaaaactacgaatcctttctgaggcgagatctttccattgttccaataaaaacctaagcaagttgaatgtggaagtcggtaagtagggagcaccccgccttctttacaccagcggacctctgggttactttctaccatgggtctcagccacatacacatacacacgcacgcactcatgtgcacacactcaatacttgagaaggatttgtgaaaatgtacatacccagtacacagatgtacacagtgctctgacagccctcaagctcttctgaggcttagcagtgatgggtccacaacatggaatactgaaagggattcactgagatctacgtgtgctaataaagtgcttgaagccagcctggtctcttccccagcatcccctagtccaaggccagctgccacacacacatggacagagaaaggcgagacaccggttacttctcctagccaactggctcattattatttgctgaatatttgctggatttttctggttttgttctgttttagaatggggtgggagtggatgttatgtcacaatcctaatacagtaaagttttgcatcttccatatcttatgcaaaaacagacatttaaatcaataaatagttgtgccctagactgaaagttaatgtttaggagagggaaaaattgttggaattttttctacatttttttgtgaagaatcttttttggaaaggaaggatacatatttttgttgtgtaatattttctatttttgaatgcattttattggtacaagactgtttttttggtgaagacattatttaaaaaaagaaaaaaagaaaaaaactaatcgaaaagtttgcccttaaggatatgctgcagttttgagattaaaaaataataactgattcaagatgcgtgttaaaagttgggattatattgttgtttttgtaattgttacaagaagaagtttgtacccactgctgtttattttgtttcagatgagtaagtaaagggattgttcttgttttattctttttttagagaaaaaagctatttatgaaatgtcaaaaacactggactgtgagtttaagtgtggaagcattttaccaccctgtgtcttcaaccaattatgggaaaccttttctctccccccctgccttagccttgccaaatgaggaaaacgtaacagctctcagatgacggaagtcaccgaagccctgctttaatttttatggtctgaaaaagtcggaaaaccaaagttaaatttgtttctgaaatcccgctgtctatagccccttttttgtacaacacagccggctggctctgcctctctatcttggatcattgccttcttaggaacgtggggccagctctgccaagaggcgtgaaggtggcgaggtcacaggaagtgaggtgtgagggggacccctagggccccggagcttctccatccagaggcgaggctgccaagagcacacacagctaacagtgcctggcggggtcgcccctgtccccctcaccttctgcttcgaagaccctccagttaccgtggctctgcctttctcagcagcccacgttccgtggatgggagggggtgggatccaagcagaaaacacggctgtgggcgctgcgaaggccccggccctcaggaggtaaagcaagggaccactcagcacaagggctcttgctgcccgcctcctctttgactctgtggatcgtccatccatctgctcactgtgaagatggagaggcagtgcgccctgaggctgttcaatagcttttccatattttttcaacattgaaaaaataatttttaaaaactgtgatatttaaaaaaaaaaaaatcatttggctggagggaagggaaaagggaaacaccaaaagctgtaacatgattaactggagatatttataactggggcactttccagaccaagacaaatgaattgttttctggacccgaaagcagccaaattttaagactgtcagtgacaaaaagctgaagagaggcctccatttctcctcctttcttctttctgtcccaaattctctcattttctcttctagcttctcttggtaactgtccaatggacttcatacttcatgcaaaatcccgcgcatgcacgcgagcgcgcacgcatgcgcgtgtacacacacacacacacacacacacacacacacacacacacacaagcaaaaaaaaaaactatttttcttaaggattgtgggactaaatttaaagtcatgtgccttcatttttttcccttttatagttaaatgaacctcttccttttttacaatgtgttgggttttgttttgtttttagtagaaggggaaggttaaagtgtttgtggaagagaggatttttaggcatcaactgggagatttttttagcatattcccccactaaatattaaacacaaacatctcaatccctccacgtgtcactgtgcacacttagagcatcaaggaatcagaatccgacagcctaatccacttgattttagagaagttcctgaaatttctatttcctagacttttttattgttcttattttatcacagtgaggtgagcaaggcaagttgcctcgttctcccaactcggtgcttctgcttgtggggtgggggtggggcggtatagacaagggtgcactcctaaagctctctggtgctgaagggcctcaaggttgagtttctttcagaaaatgtgtatggcacactctcaagtgcacacgtgaacggtgtcatgcgcactatttttaaaggacaagggaaggggctctgaagtgggttttgctttctctcatgacatttgatttccctggtgccttattcctattctatgctggcactcacatgcccacaggaacacacgctgatgtcagccccaggagtgaggacctctaggtgacagttgagcatgtggggaccatcggatattggggtcagttggtaggggaggaactagatggctgaaaatacacagggactgcacaagcccatcacagtcaagattagtaatgctcatatgtgagtatgtgcaatacatgcacacacaaacacacacacagacacacacagagatgcacacacaaacaccaaatacactcttcttcctctgaacattgcttccttcttcatttcctgtctttgccttctcataaaggtgctgcttgctgctgctgctgaggtgcccggagtccagaatgcccagtaatcactcaggcacaagcctggcactgccacgttcagtccttggcaagaccaaaccctggtttctcttgcctggggctgaaaaccgtcagatttttctcatcaaaaaaaaaaaaaaaaagttatccaaggaatcagtggattatagttactctgcattaaaaatgcactttaaaaataaataaaagctccagactgtttaaaacacacagagggaacaggagaaagataaacgtgctagtgtctgaacccagttcagcatatctccagttgaaacagtatacactatattatgtataaatgtatacacacttctatatatgtccacatatatgcggtgtgtgtattatacaggtataggtgtgtgtgcacgcacacaggtgcacatagcatatcaagtgttcattacaaatccagatgctcatttcacaaacagcagcagaggaaacaaggttggactcttgcagcagatcacaaaacaataaaaacagccacttgcggtgacgctggtcactgctgtgtgttcataaggaatggattgtaacaaaggaaaacaaggagcagtgttagcaattgaggaaaactgggacagaccatcttgatccaatgggaatgcagtaatgttctctaccatttcatccgttctttctgttagtcgtgacgatttgattttcatttttgcctattaaaaatggtttagattcaagtgaccacatccaagtgaacaaaacaaccacagtgaaagtccttttcagtaggaagatgtcagaaaactcaaaacccttggcctggctcagaactaccatgtgcaaaccagaactctctcaacgtttgaaataaaaactttaaaactctttttgaagcaccttaacgtggccatccatttgacaagtgggtgccacctttttctttgagcaccttattgacgtattttgctatctgctgtcttctgttactgttggctgaatagctagctgttaacacacacacatgtgcacagaccagacatctgagcatgcgtgttctcaatgacgtttaccgtggtgactgctggaaggtgaactcattttctgatttgcccaccacagtgttgtgataagactcgaagaaaccctgccctgcacggaaaaatgtcccttatcacgttgtatattagggtgggaaggaatatggtcccctttttgcaattgctactgtgtatacatacacatgcacacacacacacacacacacacacacacacacacacacacacacacacactgtatattcagacatgatgtacacacacaaacataactcatttgtccaagtgatatttcagatgtttctgtgggtgtcacacaccatgtgcagttttccacttcccagagaattttgagtggagggtaacttttcagactgatgaacggggcactgaggaagagtttgaagtgggaggcaagaaaggagagagcattaagtcaaaagaataatttcccaagagaagctggaggaatggctgtccttgcaggtgggtgtggaacactgctgtctcagtctgcactgtagaaatccatgcacacatcaacacacacacacacacacacacacacacacatacacacatccccccacaggggcgtggtctgggatgagcaggtcaatagttttgagagggagtttgttccttttttttctctcattatactcttgtcagttattaaacaaacaaacagaaaaaaattgttttgaaaaaccttgcgtacgccttttctatcaagtgctttaaaatatagactaaatacacacatcctgccagtttttcttacagtgacagtacccttacctgccatttaatattagcctcgtatttttctcacgtatatttacctgtgacttgtatttgttatttaaacaggaaaaaatttcaaaaaaaagaaaaattaactgtagcgcttcattatactattatattattattattgtgacattttggaatactgtgaagttttatctcttgcatatactttatacagaagtattacgccttaaaaatacgaaaataaattttacaaggtttctgttttgtgtggaagagtaattgatgttgctaagaatgatgtttgttttttggggtttttgttgtttttttttttaaatgttaccagcactttttttgtaagtttcactttctgaggtattgtacaagttcacactgtttgtgaagtttgaatatgaaggaataattaa